(19) United States
(12) Patent Application Publication
(10) Pub. No.: US 2026/0226147 A1
(43) Pub. Date: Aug. 6, 2026

(54) ANTI-CSP ANTIBODY VARIANTS

(71) Applicant: ATRECA, INC., San Carlos, CA (US)

(72) Inventors: Daniel Eric EMERLING, San Carlos, CA (US); Katherine L. WILLIAMS, San Carlos, CA (US); Steve GUERREO, San Carlos, CA (US)

(21) Appl. No.: 19/153,212

(22) PCT Filed: Feb. 2, 2024
(86) PCT No.: PCT/US2024/014226

§ 371 Date: Aug. 1, 2025

Related U.S. Application Data
(60) Provisional application No. 63/483,052, filed on Feb. 3, 2023.
(60) Provisional application No. 63/542,164, filed on Oct. 3, 2023.

(51) Int. Cl. C07K 16/20; A61K 39/00; A61P 33/06; G01N 33/68

(52) U.S. Cl. CPC C07K 16/205; A61P 33/06; G01N 33/6854; A61K 2039/505; C07K 2317/34; C07K 2317/565; C07K 2317/92; C07K 2317/94; G01N 2333/445

(57) ABSTRACT

The present disclosure provides anti-circumsporozoite (CSP) antibodies, compositions comprising such antibodies. Also disclosed are methods of producing the disclosed antibodies and methods of treating or preventing malaria using the same. Also disclosed are methods of selecting an antibody as an anti-malaria therapeutic antibody.

### CROSS REFERENCE TO RELATED APPLICATIONS

This application claims priority to U.S. Provisional Application No. 63/483,052, filed on Feb. 3, 2023, and U.S. Provisional Application No. 63/542,164, filed on Oct. 3, 2023. The entire content of said provisional applications is herein incorporated by reference for all purposes.

### SEQUENCE LISTING

The instant application contains a Sequence Listing which has been submitted herewith and is hereby incorporated by reference in its entirety. Said .xml copy, created on Apr. 5, 2024 is named 097519-1425106-3800PCT, and is 1,146,419 bytes in size.

### FIELD

The present disclosure relates to compositions for treating or preventing malaria, and to antibodies conferring protection against infection by malarial parasites such as Plasmodium falciparum by insect vector transmission. The present disclosure also relates to methods for treating, preventing, or diagnosing Plasmodium infection in a mammal.

### BACKGROUND

Malaria causes a large burden of morbidity and mortality, especially in the developing world. The causative agent of malaria is a protozoal parasite, which is transmitted by mosquitoes. Several infectious Plasmodium species cause malaria, the deadliest of which is Plasmodium falciparum. Others include P. vivax, P. ovale, and P. malariae. Over 80% of malaria-attributable deaths are in children under five. The most advanced vaccine, and the only one recommended for use by the WHO, RTS,S/AS01 (Mosquirix™), targets the circumsporozoite protein (CSP) of Plasmodium falciparum (Pf), the malaria species primarily responsible for mortality in Africa. Three immunizations with RTS,S/AS01 induce anti-PfCSP antibodies that act by binding to sporozoites, the infective form of the malaria parasite introduced by mosquito bite, and inhibiting their initial infection of liver cells. However, the immune response induced by Mosquirix™ in children is limited to 45% vaccine efficacy against clinical malaria after the first dose, waning to 36% over 4 years of follow-up. Thus, other immunization approaches will be needed to achieve the WHO's goal of reducing the malaria case incidence and mortality rates by 90% by 2030.

Recent reports show that treatment with mAbs can completely prevent malaria after controlled infection and provide 88% efficacy for 6 months (prevention of infection) in an endemic region. Thus, mAbs with durability lasting 4-6 months could provide an intervention with greater protective efficacy than seen with RTS,S, significantly aiding efforts to prevent seasonal transmission. The mAbs tested in clinical trials, L911 and CIS438,9, were isolated from B cells of vaccinees immunized with whole sporozoites, and can prevent malaria infection by targeting specific epitopes on CSP. Given the published support for prophylaxis with mAbs as a strategy against malaria, and the productive advancement of mAbs as therapeutics and prophylactics against infectious diseases in general, the need exists to generate additional protective anti-CSP antibodies with improved therapeutic profiles.

### SUMMARY

The present disclosure provides anti-circumsporozoite (CSP) antibodies, compositions comprising such antibodies. Also disclosed are methods of producing the disclosed antibodies and methods of treating or preventing malaria using the same. Also disclosed are methods of selecting an antibody as an anti-malaria therapeutic antibody.

In certain non-limiting embodiments, the present disclosure provides recombinant anti-circumsporozoite (CSP) antibodies that bind to a first epitope present in the central repeat region of CSP and bind to a second epitope of CSP. In certain embodiments, the first epitope includes the amino acid sequence NPNA (SEQ ID NO: 923). In certain embodiments, the first epitope consists of the amino acid sequence selected from SEQ ID NOs: 923-974.

In certain embodiments, the second epitope is heterologous to epitopes present in the RTS, S vaccine. In certain embodiments, the second epitope includes a minor repeat region of CSP and/or a junctional region of CSP. In certain embodiments, the second epitope includes a DPNA (SEQ ID NO: 977)/NPNV (SEQ ID NO:980)-containing minor-repeat amino acid sequence and/or a DPNA (SEQ ID NO: 977)/NPNV (SEQ ID NO:980)-containing junctional amino acid sequence. In certain embodiments, the second epitope consists of the amino acid sequence selected from SEQ ID NOs: 975-1195.

In certain embodiments, the antibody binds to at least one additional epitope of CSP. In certain embodiments, the at least one additional epitope comprises a DPNA (SEQ ID NO: 977)/NPNV (SEQ ID NO:980)-containing minor-repeat amino acid sequence and/or a DPNA (SEQ ID NO: 977)/NPNV (SEQ ID NO:980)-containing junctional amino acid sequence. In certain embodiments, the at least one additional epitope consists of an amino acid sequence selected from the group consisting of SEQ ID NOs: 975-1195.

In certain embodiments, the recombinant antibody includes a heavy chain variable region (VH) including an amino acid sequence that is at least about 80% identical to the amino acid sequence selected from SEQ ID NOs: 1-461. In certain embodiments, the recombinant antibody includes a VH including an amino acid sequence selected from the group consisting of SEQ ID NOs: 1-461.

In certain embodiments, the recombinant antibody includes a light chain variable region (VL) including an amino acid sequence that is at least about 80% identical to an amino acid sequence selected from the group consisting of SEQ ID NOs: 462-922. In certain embodiments, the recombinant antibody includes a VL including an amino acid sequence selected from the group consisting of SEQ ID NOs: 462-922.

In certain embodiments, the recombinant antibody includes VH including an amino acid sequence that is at least about 80% identical to an amino acid sequence selected from the group consisting of SEQ ID NOs: 1-461, and a VL including an amino acid sequence that is at least about 80% identical to an amino acid sequence selected from the group consisting of SEQ ID NOs: 462-922. In certain embodiments, the recombinant antibody includes a VH including an amino acid sequence selected from the group consisting of SEQ ID NOs: 1-461, and a VL including the amino acid sequence selected from SEQ ID NOs: 462-922.

In certain embodiments, the recombinant antibody includes a heavy chain variable region (VH) and a light chain variable region (VH), wherein the heavy chain variable region includes a CDR1, a CDR2, and a CDR3 of the heavy chain variable sequence set forth in SEQ ID NOs: 1-461, and the light chain variable region includes a CDR1, a CDR2, and a CDR3 of the light chain variable sequence set forth in SEQ ID NOs: 462-922. In certain embodiments, the recombinant antibody includes a heavy chain variable region (VH) and a light chain variable region (VH) set forth in Table 3.

In certain embodiments, the antibody exhibits at least 20% reduction in parasite liver load as compared to a reference antibody. In certain embodiments, the antibody exhibits at least 20% increase in survival rate as compared to a reference antibody. In certain embodiments, the antibody exhibits increased conformational stability as compared to a reference antibody. In certain embodiments, the antibody exhibits increased colloidal stability as compared to a reference antibody. In certain embodiments, the reference antibody is AB-000317, AB-000224, or AB-007088.

In certain non-limiting embodiments, the present disclosure relates to a polynucleotide encoding a presently disclosed antibody. In certain non-limiting embodiments, the present disclosure relates to an expression vector and/or a host cell including the polynucleotides disclosed herein.

In certain non-limiting embodiments, the present disclosure also relates to compositions including the antibodies disclosed herein. In certain embodiments, the compositions further include a pharmaceutically acceptable carrier.

In certain non-limiting embodiments, the present disclosure relates to methods of preventing or treating malaria in a subject in need thereof including administering an effective amount of the antibodies or the compositions disclosed herein. In certain embodiments, the patient is a pediatric patient.

In certain non-limiting embodiments, the present disclosure relates to methods of selecting an antibody as an anti-malaria therapeutic antibody. In certain embodiments, the methods include: a) analyzing the antibody for binding to a first epitope of the central repeat region of CSP; and b) analyzing the antibody for binding to a second epitope of CSP that is heterologous to epitopes present in the RTS,S vaccine; wherein the antibody is selected if it binds to both the first epitope and the second epitope. In certain embodiments, the methods further comprise: c) analyzing the antibody for binding to at least one additional epitope of CSP that is heterologous to epitopes present in the RTS,S vaccine; wherein the antibody is selected if it binds to the first epitope, the second epitope, and the at least one additional epitope

In certain non-limiting embodiments, the present disclosure relates to methods of selecting an antibody as an anti-malaria therapeutic antibody. In certain embodiments, the methods include selecting the antibody if i) the antibody binds to a first epitope of the central repeat region of CSP, and ii) the antibody binds to a second epitope that is heterologous to epitopes present in the RTS,S vaccine. In certain embodiments, the methods include selecting the antibody if i) the antibody binds to a first epitope of the central repeat region of CSP; ii) the antibody binds to a second epitope that is heterologous to epitopes present in the RTS,S vaccine; and iii) the antibody binds to at least one additional epitope that is heterologous to epitopes present in the RTS,S vaccine.

In certain embodiments, the first epitope includes the amino acid sequence NPNA (SEQ ID NO: 923). In certain embodiments, the first epitope consists of the amino acid sequence selected from SEQ ID NOs: 923-974. In certain embodiments, the second epitope is heterologous to epitopes present in the RTS, S vaccine. In certain embodiments, the second epitope includes a minor repeat region of CSP and/or a junctional region of CSP. In certain embodiments, the second epitope includes a DPNA (SEQ ID NO: 977)/NPNV (SEQ ID NO:980)-containing minor-repeat amino acid sequence and/or a DPNA (SEQ ID NO: 977)/NPNV (SEQ ID NO:980)-containing junctional amino acid sequence. In certain embodiments, the second epitope consists of the amino acid sequence selected from SEQ ID NOs: 975-1195. In certain embodiments, the first epitope consists of the amino acid sequence selected from SEQ ID NOs: 923-974 and the second epitope consists of the amino acid sequence selected from SEQ ID NOs: 975-1195.

In certain embodiments, the antibody binds to the first epitope with a binding affinity (K_{D}) that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M. In certain embodiments, the antibody binds to the second epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M. In certain embodiments, the antibody binds to the first epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M; and to the second epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.

In certain embodiments, a) the first epitope consists of the amino acid sequence selected from SEQ ID NOs: 923-974; b) the second epitope consists of the amino acid sequence selected from SEQ ID NOs: 975-1195; and c) the at least one additional epitope consists of the amino acid sequence selected from SEQ ID NOs: 975-1195.

In certain embodiments, the antibody binds to the at least one additional epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M. In certain embodiments, the antibody binds a) to the first epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M; b) to the second epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M; and c) to the at least one additional epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.

### BRIEF DESCRIPTION OF THE DRAWINGS

FIGS. 1A-1G show that functional antibodies bind CSP-derived peptides not present in the RTS,S vaccine. FIG. 1A shows percent inhibition in the sporozoite liver burden mouse model and number of nucleotide mutations from germline are shown for mAbs reactive to NANP6 (SEQ ID NO: 1198) repeat-region peptide (circles, n=67) or to the C-terminal region peptide (squares, n=10) and are indicated as originating from vaccinees who were protected (green) or unprotected (blue) and who received the standard (012M, closed symbols) or the fractional (Fx017M, open symbols) dose. FIG. 1B shows SPR-determined binding potencies (KD) of antibodies (n=141) selected from 35 of the most efficacious lineages tested against CSP and a panel of CSP-derived peptides that are homologous (NANP6 (SEQ ID NO: 1198), NPNA3 (SEQ ID NO: 957)) or heterologous (NVDP3NANP2 (SEQ ID NO: 1200), NPDPNANPNVDPNANP (SEQ ID NO: 1183), Junction) to RTS,S. Examples are shown of an antibody with a broadly promiscuous binding profile (green, AB-007163), an antibody with a profile relatively biased to homologous peptides (purple, AB-007143), and one with a profile in between these extremes (blue, AB-007175). FIGS. 1C-1G show linear regression comparing the number of nucleotide mutations from germline (SHM) per heavy chain versus log-transformed SPR binding off-rate (k_{off}) against peptides, with FIG. 1C showing short, major repeat (NPNA3 (SEQ ID NO: 957), n=140), FIG. 1D showing junctional (KQPADGNPDPNANPN (SEQ ID NO: 1199), n=68), FIG. 1E showing short, minor repeat (NPDPNANPNVDPNANP (SEQ ID NO: 1183), n=109), FIG. 1F showing long, major repeat (NANP6 (SEQ ID NO: 1198), n=141), and, FIG. 1G showing long, minor repeat (NVDP3NANP2 (SEQ ID NO: 1200), n=129). For correlations of non-transformed data, P<0.03 for all comparisons (Spearman test), and P<0.04 for all comparisons (Pearson test) except P>0.5 for NVDP3NANP2 (SEQ ID NO: 1200).

FIGS. 2A-2M show in vivo pharmacology, SHM, and binding of anti-CSP antibodies. FIGS. 2A-2C show data from a liver burden model with FIG. 2A showing percent inhibition of n=69 antibodies (32 lineages) compared to AB-000317 with colors other than grey indicating the six lineages that contain the most efficacious antibodies (*P>0.05, †P<0.05, two-sided, non-parametric log-rank), FIGS. 2B-2C showing data from AB-000224 and AB-000317 of parasite bioluminescence in the liver (total flux, photons/sec) (FIG. 2B), *P<0.035, and serum concentrations (Serum [Ab], μg/ml) of antibody at time of sporozoite challenge (FIG. 2C), lines and bars indicate geometric mean and geometric standard deviation, P>0.2 (“ns”), two-tailed Mann-Whitney test. FIGS. 2D-2I show percent liver burden inhibitory activity normalized to the activity of AB-000317 with each antibody indicated as having activity significantly better (dark blue triangle), not different (green circles), or weaker (light blue triangles) than AB-000317 (two-sided, non-parametric log-rank) versus FIGS. 2D-2G showing binding off-rate (SPR, koff) against, CSP (n=70) (FIG. 2D), major repeat (NPNA3 (SEQ ID NO: 957), n=70) (FIG. 2E), junctional (KQPADGNPDPNANPN (SEQ ID NO: 1199), n=42) (FIG. 2F), and minor repeat (NPDPNANPNVDPNANP (SEQ ID NO: 1183), n=60) (FIG. 2G) peptides, and versus, FIGS. 2H-2I showing the number of amino acid residue changes from SHM for each antibody (n=70), heavy (FIG. 2H) and light chains (FIG. 2I), linear regression of log-transformed data is shown. For correlations of non-transformed data, P<0.005 for all comparisons (Pearson test), and P<0.05 for all comparisons (Spearman test) except P>0.06 for liver burden inhibition versus koff [NPDPNANPNVDPNANP] (SEQ ID NO: 1183). FIG. 2J shows SPR binding off-(k) versus on-rates (kon) against NPNA3 (SEQ ID NO: 957) peptide of antibodies with high SHM (green, ≥20 mutations per clone, n=56) or low SHM (blue, <20 mutations per clone, n=14) and with activity weaker (down triangles), no different (circles), or better (up triangle) than AB-000317 (two-sided, non-parametric log-rank). Monoclonal antibodies from a lineage reported to bind CSP with Fab-Fab homotypic interactions indicated (red circles, AB-00039942 AB-007159, AB-007160, AB-007161). FIG. 2K shows hazard ratios of n=25 antibodies (14 lineages) compared to AB-000317 in the mosquito-bite parasitemia model with colors other than grey indicating the five lineages that contain the most efficacious antibodies, and, FIGS. 2L-2M show survival curves from repeat experiments in comparison to AB-000317 of AB-000224 [0.74 (0.15, 3.8)] (FIG. 2L) and AB-007088 [0.61 (0.097, 3.8)] (FIG. 2M), two-sided, non-parametric log-rank [Mantel-Haenszel hazard ratio (95% confidence intervals)].

FIGS. 3A-3H illustrate CSP-reactive lineages from blood PBs after the third dose of RTS,S. FIGS. 3A and 3B show IgG lineages for each vaccinee (bars, n=45) that are clonally expanded (i.e. have ≥2 distinct nucleotide clones; green), that are cellularly expanded but with only one IgG clone observed (i.e. have ≥2 identical nucleotide clones; grey), or that lack evidence of recent expansion and contain only one observed PB (blue) are shown by, FIG. 3A, number of lineages or, FIG. 3B, number of PBs per vaccinee. FIG. 3C shows, by vaccinee, the size of each expanded lineage was calculated by dividing the number of PBs in that lineage by the number of PBs in all expanded lineages within each repertoire and then assigning a rank-size. Boxes indicate interquartile ranges, lines within boxes are medians, and whiskers represent minimum and maximum across vaccinees for each rank-size, with the top four rank-size lineages containing 33% of PBs in all expanded lineages (dotted line). FIGS. 3D-3G, ELISA reactivity, SHM levels and vaccinee protection status of mAbs from expanded lineages (n=349). FIG. 3D shows number of nucleotide mutations from germline (SHM) for mAbs that are not reactive (dark blue, n=185), show indeterminant, weak signal (orange, n=29), or are reactive (light green, n=135) in a CSP ELISA. Domain specificity for CSP-reactive mAbs is shown in the light green box. Monoclonal antibodies reactive by ELISA to NANP6 repeat-region peptide (light green, n=98), to the C-terminal region peptide (Pfs16, light blue, n=20), or that are not reactive in either peptide ELISA (light green, n=9), lines are medians, ***P<0.0001, **P<0.001, unpaired two-tailed Mann-Whitney test. CSP-reactive mAbs that were not tested in peptide ELISAs (n=8) are not shown. FIGS. 3E-3G show percent of tested antibodies from expanded lineages that originate from protected (green, n=36) and not protected (blue, n=9) vaccinees that are, CSP-reactive (82/249 and 53/100 mAbs, respectively; FIG. 3E), repeat-region, NANP6 (SEQ ID NO: 1198) peptide-reactive (59/244 and 39/97 mAbs, respectively; FIG. 3F), and, the subset from just the dominant rank-size 1-4 lineages that are CSP-reactive (52/142 and 31/46 mAbs, respectively; FIG. 3G), **P<0.001, *P<0.01, Fisher's exact test. In FIG. 3H, for vaccinees shown on the x-axis, each symbol indicates a single lineage. The lineages (n=369) from which a clone was selected for testing are indicated by CSP reactivity: CSP-reactive (green dots, n=139), indeterminant (orange dots, n=29), or not reactive (blue triangles, n=201). All lineages that were not tested are shown (grey circles, n=13,134; 2,313 expanded and 10,821 single-PB lineages). Protected vaccinees have a lower ratio of CSP-reactive versus non-reactive lineages than not protected vaccinees (bootstrap analysis, P=0.0011). Red circles indicate the two lineages that contain the amino acid sequence of AB-000317.

FIG. 4 shows histogram of the number of nucleotide mutations from germline (SHM) of combined IgG heavy and light chains from blood PBs collected 7 days after administration of the third dose (blue, n=22,319) or fourth dose (grey, n=10,429) of RTS,S.

FIGS. 5A-5I show IgG sequence and repertoire features of the PB response after the third dose of RTS,S. FIGS. 5A-5C show germline V-gene usage compared between protected (green, n=36) and not protected (blue, n=9) subjects and across dose groups (standard dose group, “012M”, n=15; fractional dose group, “Fx017M”, n=30) for heavy (FIG. 5A) and light (FIG. 5B) chains, with IGHV3-30, IGHV3-33, KV1-5, KV3-20, and LV1-40 showing high prevalence. FIG. 5C shows specific pairings of heavy and light chain genes. FIG. 5D shows three heavy chain germline genes, IGHV3-73, IGHV4-61, and IGHV5-51, were initially associated with vaccinees' protection status (P<0.05, Wilcoxon Rank Sum test) but were not associated after correcting for multiple hypothesis testing. All were P>0.05, Benjamini-Hochberg or Bonferroni tests. No significant associations were detected between vaccinees' protection status and dose groups for, FIGS. 5E-5F, IgG heavy and light chain constant region subclass (P>0.05 for all analyses, Wilcoxon Rank Sum test), FIG. 5G, repertoire clonality for analyses that included the lineages that contain only one PB (Normalized Shannon entropy, P>0.05 for all analyses, Wilcoxon rank sum test or Kolmogorov-Smirnov test), and length of the heavy (FIG. 5H) or light (FIG. 5I) chain complementarity determining region 3 (CDR3, P>0.05 for both, Wilcoxon Rank Sum test). Boxes indicate interquartile ranges, lines within boxes are medians, whiskers represent farthest data points within 1.5× the interquartile range, and points outside whiskers are plotted individually as outliers.

FIGS. 6A-6C show antibody lineages tested in binding assays and reactivity to CSP or HBsAg. Rank-size of expanded lineages in each protected and not protected vaccinee repertoire from PBs collected 7 days after the third dose of RTS,S. FIG. 6A shows expanded PB antibody lineages (circles representing ≥1 lineages) for each rank-size and vaccinee from which a mAb was selected, recombinantly expressed and screened (n=349 mAbs, 282 circles) in the CSP ELISA. Circle sizes are proportional to the fraction of lineages tested among all lineages observed at each rank-size and vaccinee. Lineages from a vaccinee that have the same number of PBs have the same rank-size. The largest circles indicate all lineages from the vaccinee at that rank-size were tested. The smallest circle indicates only one 1 of the 58 lineages observed from the vaccinee at that rank-size was tested. In some cases, none of the lineages from a vaccinee at an indicated rank-size were tested (grey bars). FIGS. 6B and 6C show expanded PB antibody lineages (circles representing 1-5 lineages) for each rank-size and vaccinee from which a mAb was tested in, FIG. 6B, a CSP ELISA, reactive (green, n=135 mAbs, 94 circles), indeterminant (grey, n=29 mAbs, 14 circles), not reactive (blue, n=185 mAbs, 144 circles), or a combination of these outcomes for different mAbs from lineages of same rank-size and vaccinee (pie charts of mixed colors, 30 circles), and tested in, FIG. 6C, an HBsAg ELISA, reactive (green, n=38 mAbs, 36 circles), indeterminant (grey, n=3 mAbs, three circles), not reactive (blue, n=77 mAbs, 72 circles), or a combination of these outcomes for different mAbs from lineages of same rank-size and vaccinee (pie charts of mixed colors, two circles).

FIGS. 7A and 7B show SHM and CSP-peptide binding of mAbs versus RTS,S dose group and vaccinee protection status. Distribution of SHM levels for mAbs from expanded lineages of vaccinees, FIG. 7A, administered a third standard dose (012M, dark blue, n=15) or a delayed fractional dose (Fx017M, orange, n=30) of RTS,S with mAbs shown reactive to CSP repeat region (NANP6 (SEQ ID NO: 1198), n=45 and n=53, respectively), to the CSP C-terminal region (C-terminal, n=4 and n=16, respectively), or neither peptide (negative, n=5 and n=4, respectively) in comparison to mAbs not reactive in the CSP ELISA (n=72 and n=113, respectively), and, FIG. 7B, from vaccinees that were protected (green, n=36) or not protected (light blue, n=9) with mAbs shown reactive to CSP repeat region (n=59 and n=39, respectively)], to the CSP C-terminal region (n=12 and n=8, respectively), or neither peptide (negative, n=6 and n=3, respectively) in comparison to mAbs not reactive in CSP ELISA (n=147 and n=38, respectively), lines are medians, ***P<0.0001, **P<0.001, *P<0.02, or P>0.1 (“ns”), unpaired two-tailed Mann-Whitney test.

### DETAILED DESCRIPTION

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

As used in this specification and the appended claims, the singular forms “a,” “an” and “the” include plural referents unless the context dictates otherwise. Thus, for example, reference to “a protein” or an “antibody” includes a plurality of proteins or antibodies, respectively; reference to “a cell” includes mixtures of cells and the like.

As used herein, the term “about” or “approximately” refers to the usual error range for the respective value readily known to the skilled person in this technical field, for example, ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of antibody or fragment thereof that exhibits the desired biological activity, e.g., binding the specific target antigen. Thus, it is used in the broadest sense and specifically covers monoclonal antibodies (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

As used herein, the terms, “anti-CSP antibody” and “CSP antibody” are used synonymously and refer to an antibody that binds to Plasmodium falciparum circumsporozoite (CSP) antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

As used herein, “V-region” refers to an antibody variable region domain comprising the segments of Framework 1, CDR1, Framework 2, CDR2, Framework 3, CDR3, and Framework 4. The heavy chain V-region, VH, is a consequence of rearrangement of a V-gene (HV), a D-gene (HD), and a J-gene (HJ), in what is known as V (D) J recombination during B-cell differentiation. The light chain V-region, VL, is a consequence of the rearrangement of a V-gene (LV) and a J-gene.

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3 numbered sequentially starting from the N-terminus, and are also identified by the chain in which the particular CDR is located. Thus, a VH CDR3 (HCDR3) is located in the variable domain of the heavy chain of the antibody in which it is found, whereas a VL CDR3 (LCDR3) is the CDR3 from the variable domain of the light chain of the antibody in which it is found. The term “CDR” is used interchangeably with “HVR” when referring to CDR sequences.

The amino acid sequences of the CDRs and framework regions can be determined using various definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, Structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1): 207-9 (2001); MacCallum et al, Antibody-antigen interactions: Contact analysis and binding site topography, J. Mol. Biol., 262(5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering is based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)). The CDRs can also be determined using available in silico systems as described in Swindells et al., Journal of molecular biology 429.3 (2017): 356-364, the content of which is incorporated by reference in its entirety.

An “Fc region” refers to the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cy2 and Cy3 and the hinge between Cy1 and Cy. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modifications that modulate effector function. Fc regions also include variants that don't result in alterations to biological function. For example, one or more amino acids can be deleted from the N-terminus or C-terminus of the Fc region of an immunoglobulin without substantial loss of biological function. Such variants can be selected according to general rules known in the art to have minimal effect on activity (see, e.g., Bowie, et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibodies (see, e.g., Angal, et al., Mol Immunol 30:105-108, 1993). In certain embodiments, the Fc region includes substitutions that improve pharmacokinetics properties of an antibody, e.g., increased serum half-life. Non-limiting examples of substitutions of the Fc region can be found in U.S. Pat. No. 8,088,376, the content of which is incorporated by reference in its entirety.

The term “equilibrium dissociation constant” abbreviated (K_{D}), refers to the dissociation rate constant (kd, time-1) divided by the association rate constant (ka, time⁻¹ M⁻¹). Equilibrium dissociation constants can be measured using any method. Thus, in certain embodiments, the antibodies of the present disclosure have a K_{D} of less than about 50 nM, typically less than about 25 nM, or less than 10 nM, e.g., less than about 5 nM, or than about 1 nM and often less than about 10 nM as determined by surface plasmon resonance analysis using a biosensor system such as a Biacore® system performed at 37° C. In certain embodiments, an antibody of the present disclosure has a KD of less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸ M, less than 5×10⁻⁹M, less than 10⁻⁹M, less than 5×10⁻¹⁰M, less than 10⁻¹⁰M, less than 5×10⁻¹¹M less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower as measured as a bivalent antibody. As used herein, an “improved” KD refers to a lower KD. In certain embodiments, an antibody of the present disclosure has a KD of less than 5×10⁻⁵M, less than 10-5M, less than 5×10⁻⁶M, less than 10⁻⁶M, less than 5×10⁻⁷M, less than 10⁻⁷M, less than 5×10⁻⁸M, less than 10⁻⁸ M, less than 5×10⁻⁹M, less than 10⁻⁹M, less than 5×10⁻¹⁰M, less than 10⁻¹⁰M, less than 5×10⁻¹¹M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower as measured as a monovalent antibody, such as a monovalent Fab. In certain embodiments, an anti-CSP antibody of the present disclosure has KD less than 100 pM, e.g., or less than 75 pM, e.g., in the range of 1 to 100 pM, when measured by surface plasmon resonance analysis using a biosensor system such as a Biacore® system performed at 37° C. In certain embodiments, an anti-CSP antibody of the present disclosure has KD of greater than 100 pM, e.g., in the range of 100-1000 pM or 200-1000 pM when measured by surface plasmon resonance analysis using a biosensor system such as a Biacore® system performed at 37° C.

The term “monovalent molecule” as used herein refers to a molecule that has one antigen-binding site, e.g., a Fab or scFv.

The term “bivalent molecule” as used herein refers to a molecule that has two antigen-binding sites. In certain embodiments, a bivalent molecule of the present invention is a bivalent antibody or a bivalent fragment thereof. In certain embodiments, a bivalent molecule of the present invention is a bivalent antibody. In certain embodiments, a bivalent molecule of the present invention is an IgG. In certain embodiments, monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” as used herein refer to the binding of both antigen-binding sites of a bivalent molecule to its antigen. In certain embodiments, both antigen-binding sites of a bivalent molecule share the same antigen specificity.

The term “valency” as used herein refers to the number of different binding sites of an antibody for an antigen. A monovalent antibody includes one binding site for an antigen. A bivalent antibody (e.g., a bivalent IgG antibody) includes two binding sites for the same antigen.

The term “affinity” as used herein refers to either the single or combined strength of one or both arms of an antibody (e.g., an IgG antibody) binding to either a simple or complex antigen-expressing one or more epitopes. As defined here, the term “affinity” does not imply a specific number of valencies between the two binding partners.

The phrase “specifically (or selectively) binds” to an antigen or target or “specifically (or selectively) immunoreactive with,” when referring to a protein or peptide, refers to a binding reaction whereby the antibody binds to the antigen or target of interest with an affinity that can be distinguished from non-specific interactions occurring between two proteins.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including, without any limitation, BLAST, BLAST-2, ALIGN, or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). In certain embodiments, BLAST 2.0 can be used with the default parameters to determine percent sequence identity.

A “substitution,” as used herein, denotes the replacement of one or more amino acids or nucleotides by different amino acids or nucleotides, respectively.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile, (vi) hydrophobic sulfur-containing amino acids Met and Cys, which are not as hydrophobic as Val, Leu, and Ile, (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid refers to the charge at pH 6-7.

As used herein, the terms “nucleic acid” and “polynucleotide” are used interchangeably and as used herein refer to both sense and anti-sense strands of RNA, cDNA, genomic DNA, and synthetic forms and mixed polymers of the above. In certain embodiments, a polynucleotide refers to a polyribonucleotide, polydeoxynucleotide or a modified form of either type of nucleotide, and combinations thereof. The terms also include, but are not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitutions of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular, and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector,” as used herein, refers to a recombinant construct in which a nucleic acid sequence of interest is inserted into the vector. Certain vectors can direct the expression of nucleic acids to which they are operatively linked. Such vectors are referred to herein as “expression vectors”.

The terms “host cell,” “host cell line,” and “host cell culture” are used interchangeably and refer to cells into which exogenous nucleic acid has been introduced, including the progeny of such cells. A host cell can be a recombinant host cell and includes the primary transformed cell and progeny derived therefrom without regard to the number of passages.

A polypeptide “variant,” as the term is used herein, is a polypeptide that typically differs from a polypeptide specifically disclosed herein in one or more substitutions, deletions, additions, and/or insertions. In the present invention, a “variant” with reference to the sequences described in the “Anti-CSP Antibody Variants” section refers to an engineered sequence, rather than a naturally occurring sequence.

As used herein, “recombinant antibody” refers to an antibody wherein the exact amino acid sequence of the antibody is not naturally found in a given organism (e.g., an antibody from a mammal). In certain embodiments, this term can refer to an antibody including one or more amino acid residues that are not found in a naturally occurring antibody. In certain embodiments, a recombinant antibody can have a CDR including an amino acid residue that is not found in a naturally occurring antibody (e.g., an antibody from a mammal). In another exemplary embodiment, a recombinant antibody can have a framework (FR) including an amino acid residue that is not found in a naturally occurring antibody (e.g., an antibody from a mammal). In certain embodiments, a recombinant antibody can have a constant region including an amino acid residue that is not found in a naturally occurring antibody (e.g., an antibody from a mammal).

The term “comparable,” in the context of describing the strength of binding of two antibodies to the same target, refers to two dissociation constant (KD) values calculated from two binding reactions that are within three (3) fold from each other. In certain embodiments, the ratio between the first KD (the KD of the binding reaction between the first antibody and the target) and the second KD (the KD of the binding reaction between the second antibody and the target) is within the range of 1:3 or 3:1, endpoints exclusive. A lower KD value denotes stronger binding. For example, without any limitation, an antibody variant that has stronger binding as compared to AB-000224 binds to the target with a KD that is at least ⅓ of the KD measured against the same target for AB-000224.

### Anti-CSP Antibodies

The present disclosure provides anti-CSP antibodies and variants thereof. The malaria antibodies disclosed herein were discovered in antibody repertoires generated by Immune Repertoire Capture® (IRC®) technology from plasmablast B cells isolated from two donors enrolled in a Phase 2a study evaluating the efficacy of the RTS,S vaccine in preventing malaria infection. The IRC® technology and its use in antibody discovery is well known and disclosed in, e.g., WO 2012148497A2, the entire content of which is herein incorporated by reference. The RTS,S vaccine is a pseudo-viral particle vaccine that combines the hepatitis B surface antigen and the central repeat and C-terminal regions of the Plasmodium falciparum (P. falciparum) circumsporozoite protein (CSP). RTS,S consists of two polypeptides; RTS is a single polypeptide chain corresponding to amino acids 207 to 395 of P. falciparum (3D7) that is fused to HBsAg and S is a polypeptide of 226 amino acids that corresponds to HBsAg. Stoute, et. al., N Engl J Med; 336:86-91(1997); RTS,S Clinical Trials Partnership, PLoS Med. 11(7):e1001685, (2014), WO1993/10152.

CSP comprises three main domains: i) an N-terminus; ii) a central repeat (CR) region composed of multiple (25-40) tetrapeptides of NANP (SEQ ID NO:1203) (“major repeat”) interspersed with an NPDP (SEQ ID NO:979) tetrapeptide and 2-4 NVDP (SEQ ID NO:1202) (“minor repeat”) tetrapeptides; and iii) a C-terminal domain. The central repeat region of CSP is highly immunogenic, and in all P. falciparum strains with a CSP sequence available, the repeat region is composed of 1 NPDP (SEQ ID NO:979) repeat, 3-5 NVDP repeats (SEQ ID NO:1202), and 35-41 NANP repeats (SEQ ID NO:1203) (e.g., a total of Jan. 4, 1938 of NPDP (SEQ ID NO: 979)/NVDP (SEQ ID NO: 981)/NANP (SEQ ID NO: 924) motifs are present in the P. falciparum 3D7 strain). The repeat region begins with the junctional NPDP (SEQ ID NO:979) sequence, typically followed by three alternations of NANP (SEQ ID NO: 924) and NVDP (SEQ ID NO: 981) sequences, and continues with the remaining NANP (SEQ ID NO: 924) repeats, with most P. falciparum strains having one NVDP (SEQ ID NO: 981) interspersed in the middle of the long NANP (SEQ ID NO: 924) repeat region. Pholcharee, T. et al., J. Mol. Bio. 432:1048-1063 (2020).

Analysis of anti-CSP antibodies disclosed herein indicated an inverse relationship between the percentage of CSP-specific IgG-expressing plamablasts and protection in antibodies isolated after third dose of vaccine (P3D). These data suggest that, despite the well-reported association between anti-CSP antibodies and protection, more cells expressing anti-CSP, NANP repeat-binding antibodies may not drive greater protection. This result may indicate that the simple presence of potent, inhibitory antibodies by P3D plamablasts is insufficient for protection. Rather relative levels of such antibodies versus other repeat-binding antibodies may be important in providing consistent protection.

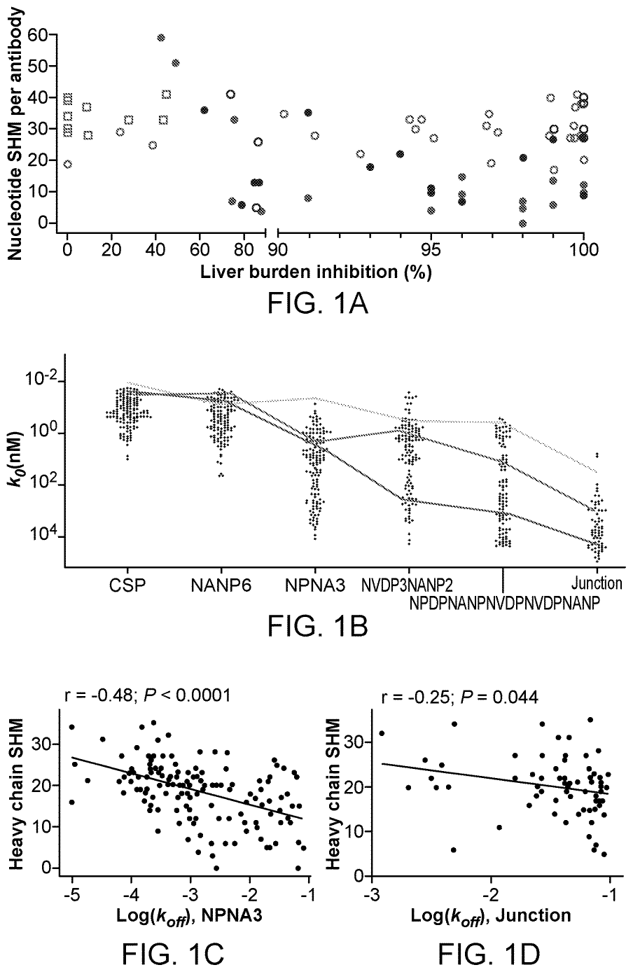

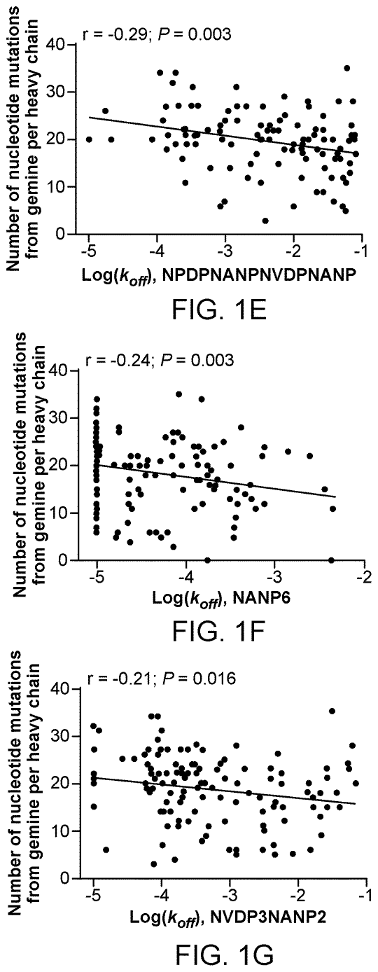

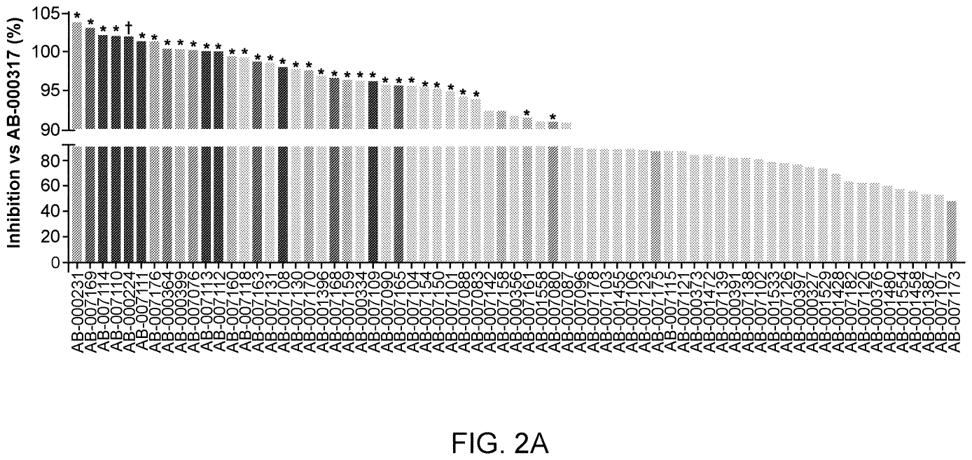

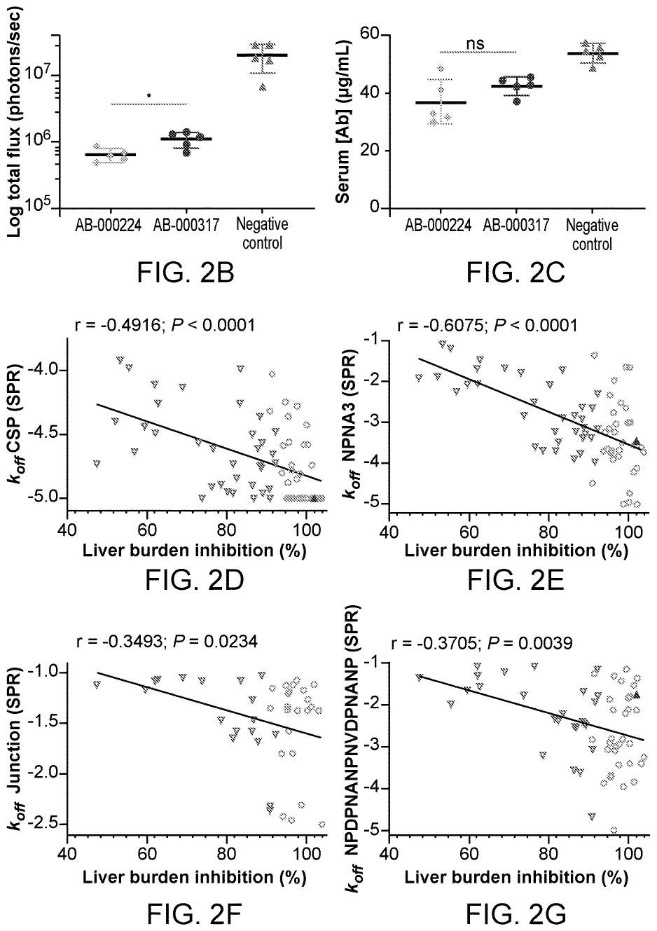

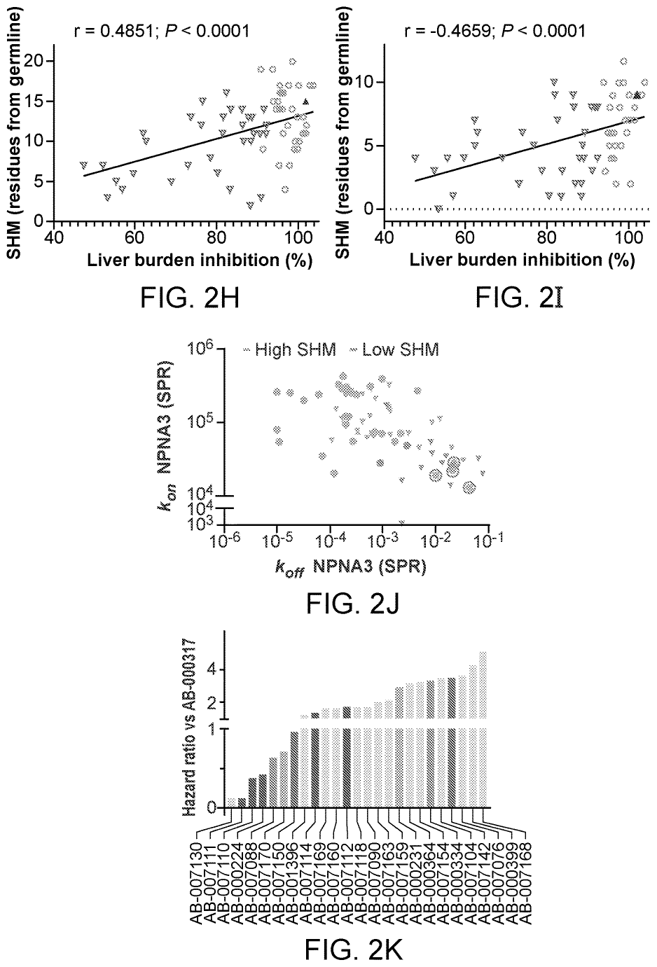

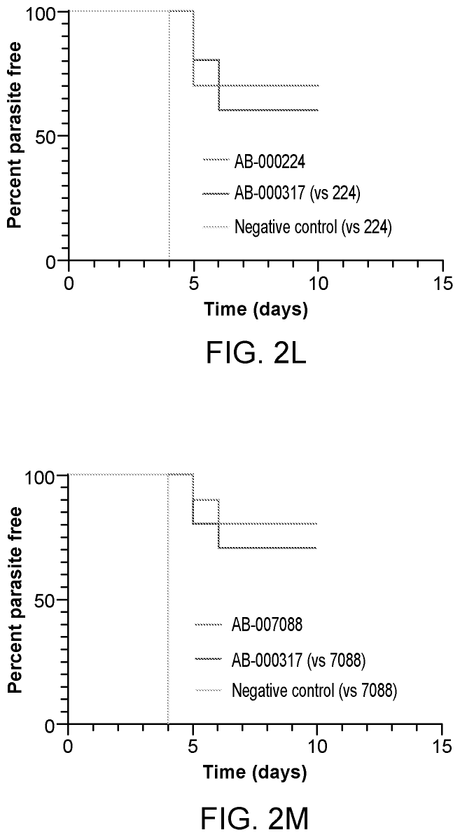

The analysis additionally indicated that antibody sporozoite inhibitory activity in vivo does not correlate with binding kinetics to the long NANP6 (SEQ ID NO: 1198) peptide (Table 9) but does significantly correlate with koff to CSP and with binding kinetics to both the short NANP (SEQ ID NO: 924)-containing peptide (NPNA3) (SEQ ID NO: 957) and peptides of minor repeat and junctional region (JR) (Table 9). These data suggest that protective antibodies induced following RTS,S vaccination affinity mature to short NANP (SEQ ID NO: 924) repeats but also gain or retain promiscuous binding activity to the minor repeat and junctional epitopes which are not present in RTS,S. Consistent with this interpretation, aggregate levels of SHM in both heavy and light chains of protective antibodies are correlated with binding kinetics to NANP (SEQ ID NO: 924)-, NVDP (SEQ ID NO: 981), and NPDP (SEQ ID NO: 979)-containing short peptides (FIGS. 1C-1E, Table 9) as well as with inhibitory activity in the sporozoite-challenge model (FIGS. 2H-2I, Table 9). Thus, affinity maturation and function appear associated with short peptide sequences both included and excluded from RTS,S but not with long NANP (SEQ ID NO: 924)-repeat sequences.

Furthermore, the correlation between in vivo activity and binding kinetics with NVDP (SEQ ID NO: 981)- and NPDP (SEQ ID NO: 979)-containing peptides absent in RTS,S (FIGS. 2F-G, Table 9), correlation between in vivo activity and binding kinetics to the short NPNA3 (SEQ ID NO: 957) but not the longer NANP6 (SEQ ID NO: 1198) peptide (FIG. 2E, Table 9); and the inverse relationship between NANP6 (SEQ ID NO: 1198)-reactivity of expanded antibody lineages and protection against CHMI, are consistent with a hypothesis where multiple NANP (SEQ ID NO: 924) repeats act as an immune “decoy” that diverts and dilutes protective immunity. Under this hypothesis, antibody lineages that solely bind to NANP (SEQ ID NO: 924) repeat-region epitopes, but provide limited protection, are preferentially expanded, diluting the protective capacity of the broader anti-CSP repertoire. Whereas promiscuous antibodies which also bind to multiple NANP (SEQ ID NO: 924) repeats and similarly exist at a high density on CSP, could further enhance antibody on-rates to heterologous epitopes. Thus, avidity afforded by promiscuous binding could drive more protective responses in vivo.

In certain embodiments, an anti-CSP antibody disclosed herein binds to a first epitope present in the central repeat region of CSP and binds to a second epitope of CSP. In certain embodiments, the central repeat region of CSP epitope comprises the amino acid sequence NPNA (SEQ ID NO: 923). Epitopes comprising NPNA (SEQ ID NO: 923) include, for example, NPNANP (SEQ ID NO: 933), NANPNA (SEQ ID NO: 931), ANPNAN (SEQ ID NO: 932), NANPNANP (SEQ ID NO: 939), ANPNANPN (SEQ ID NO: 940), NPNANPNA (SEQ ID NO: 941), PNANPNAN (SEQ ID NO: 942), (NPNA) 3 (SEQ ID NO: 957) or (NPNA) 4 (SEQ ID NO: 973).

In certain embodiments, an anti-CSP antibody disclosed herein binds, in addition to the epitope comprising NPNA (SEQ ID NO: 923), a second epitope that is heterologous to epitopes present in the RTS, S vaccine, as referred to herein as heterologous epitopes. Heterologous epitopes include epitopes of the minor repeat region of CSP, including epitopes comprising DPNA (SEQ ID NO: 977)/NPNV (SEQ ID NO:980) and epitopes of the junctional region of CSP, including epitopes comprising DPNA (SEQ ID NO: 977).

### Anti-CSP Antibodies and Variants Thereof

In certain embodiments, the present disclosure provides anti-CSP antibody variants of the antibodies isolated from the human subjects. In certain embodiments, the variants exhibit protective effects in vivo, e.g., as shown by a reduction in parasite number in a mouse model of malaria infection.

In certain embodiments, the anti-CSP variants disclosed herein maintain the binding specificity, activity and stability and/or manufacturing properties of the parental antibody. In certain embodiments, the anti-CSP variants disclosed herein generated have improved developability, e.g., as identified through various in vitro assays, such as aggregation assessment by HPLC or UPLC, hydrophobic interaction chromatography (HIC), polyspecificity assays (e.g., baculovirus particle binding), self-interaction nanoparticle spectroscopy (SINS), or mass spec analysis after incubation in an accelerated degradation condition such as high temperature, low pH, high pH, or oxidative H2O2. Mutations are successful if the activity is maintained (or enhanced) while removing or reducing the severity of the liability.

Antibody liabilities are further described in Table 1 below:

TABLE 1
Description of potential development liabilities

| | | | |
|---|---|---|---|
| Free cysteine¹ | Yield, heterogeneity, | sequence comprises an | High |
| | stability, activity | odd number of cysteines | |
| N-linked glycosylation | Yield, heterogeneity, activity | N(—P)(S, T)² | High |
| Abnormal net charge | Platform fit, PK | Sharma 2014³ | High |
| Patches of | Stability, PK | Sharma 2014 | High |
| hydrophobicity | | | |
| Patches of same charge | Stability, PK | N/A (based on structure) | Medium |
| Proteolysis | Stability, PK | (K, R)(K, R)⁴ | Medium |
| Proteolysis | Stability, PK | DP | Medium |
| Asparagine deamidation | Heterogeneity, stability, activity | NG; | Medium; |
| | | N(A, N, S, T)⁵ | Low |
| Aspartate isomerization | Heterogeneity, stability, activity | DG; | Medium; |
| | | D(A, D, S, T)⁶ | Low |
| Lysine glycation | Heterogeneity, stability, activity | K | Low |
| Methionine oxidation | Heterogeneity, stability, activity | M | Low |
| Tryptophan oxidation | Heterogeneity, stability, activity | W | Low |

¹“Free cysteine” refers to a cysteine that does not form a disulfide bond with another cysteine and thus is left “free” as thiols. The presence of free cysteines in the antibody can be a potential development liability. Typically, an odd net number of cysteines in the protein shows a likelihood there is a free cysteine.
²The N-linked glycosylation site is N-X-S/T, where X is any residue other than proline.
³Sharma et al., Proc. Natl. Acad. Sci. USA 111: 18601-18606, 2014.
⁴This motif consists of a K or R, followed by a K or R. Stated differently, the motif can be KK, KR, RK, or RR.
⁵The dipeptide NG poses a medium risk of development liability. The dipeptides NA, NN, NS, and NT pose a low risk of development liability. N may also exhibit low risk of liability for other successor residues, e.g., D, H, or P. Stated differently, dipeptide ND, NH, or NP poses a low risk of development liability.
⁶Similarly to the above, the dipeptide DG poses a medium risk of development liability. The dipeptides DA, DD, DS, and DT pose a low risk of development liability. D may also exhibit low risk of development liability for other successor residues, e.g., N, H, or P.

Another goal for engineering variants is to reduce the risk of clinical immunogenicity. For example, reducing the generation of anti-drug antibodies against the therapeutic antibody. In certain embodiments, the anti-CSP antibody variants have reduced immunogenicity as compared to the parental antibody.

The factors that drive clinical immunogenicity can be classified into two groups. First are factors that are intrinsic to the drug, such as sequence, post-translational modifications, aggregates, degradation products, and contaminants. Second are factors related to how the drug is used, such as dose level, dose frequency, route of administration, patient immune status, and patient HLA type.

One approach to engineering a variant to be as much like self as possible is to identify a close germline sequence and mutate as many mismatched positions (also known as “germline deviations”) to the germline residue type as possible. This approach applies for germline genes IGHV, IGHJ, IGKV, IGKJ, IGLV, and IGLJ, and accounts for all of the variable heavy (V11) and variable light (VL) regions except for part of H-CDR3. Germline gene IGHD codes for part of the H-CDR3 region but typically exhibits too much variation in how it is recombined with IGHV and IGHJ (e.g., forward or reverse orientation, any of three translation frames, and 5′ and 3′ modifications and non-templated additions) to present a “self” sequence template from a population perspective.

Each germline gene can present as different alleles in the population. The least immunogenic drug candidate, in terms of minimizing the percent of patients with an immunogenic response, would likely be one that matches an allele commonly found in the patient population. Single nucleotide polymorphism (SNP) data from the human genome can be used to approximate the frequency of alleles in the population.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for prediction of HLA binding and in vitro assessment to further identify potential epitopes.

In certain embodiments, antibody variants are additionally designed to enhance the efficacy of the antibody. Design parameters for this aspect focused on CDRs, e.g., CDR3. Positions to be mutated were identified based on structural analysis of antibody-antigen co-crystals (Oyen et al., Proc. Natl. Acad Sci. USA 114: E10438-E10445, 2017) and based on sequence information of other antibodies from the same lineage as AB-000224 or AB-007088.

### Approaches to Mutation Design

Development liabilities can be removed or reduced by one or more mutations. Mutations are designed to preserve antibody structure and function while removing or reducing development liabilities and improving function. In certain embodiments, mutations to chemically similar residues were identified to maintain size, shape, charge, and/or polarity. Non-limiting examples of mutations are described in Table 2 below:

TABLE 2

| | | | |
|---|---|---|---|
| Free cysteine | Odd #C | High | C(A, S) |
| N-linked glycosylation | N(—P)(S, T) | High | N-*(Q, D, S, A); |
| | | | (S, T)-*(A, N) |
| Proteolytic cleavage | (K, R)(K, R) | Medium | K, R-*(Q, S, A) |
| Proteolytic cleavage | DP | Medium | D(E, S, A) |
| Asparagine deamidation | NG; | Medium; | N-*(Q, S, A); |
| | N(A, N, S, T)* | Low | G-*(A, S) |
| Aspartate isomerization | DG; | Medium; | D-*(E, S, A); |
| | D(A, D, S, T)* | Low | G-*(A, S) |
| Lysine glycation | K | Low | K(R, Q, S, A) |
| Methionine oxidation | M | Low | M-*(Q, L, S, A) |
| Tryptophan oxidation | W | Low | W-*(Y, F) |
| Proteolytic cleavage | (K, R)(K, R) | Medium | K, R-*(Q, S, A) |

In certain embodiments, a variant of an anti-CSP antibody disclosed herein comprises modifications compared to parental antibody that provide improved pharmacokinetic properties, increased serum stability, stronger binding, and/or improved in vivo protective effects compared to parent. In certain embodiments, a variant of an anti-CSP antibody disclosed herein exhibits reduced immunogenicity and/or increased manufacturability as compared to the parent. In certain embodiments, a variant of an anti-CSP antibody disclosed herein has at least one modification, e.g., substitution, relative to the parent variable heavy chain or light chain sequence described herein, and has improved developability, e.g., decreased heterogeneity, increased yield, increased stability, improved net charges to improve pharmacokinetics, and/or reduced immunogenicity. In certain embodiments, a VH region or a VL region of such a variant of an anti-CSP antibody disclosed herein has at least two, three, four, five, or six, or more modifications, e.g., substitutions.

In certain embodiments, a variant of an anti-CSP antibody disclosed herein exhibits increased serum half-life as compared to the parental antibody. In certain embodiments, a variant of an anti-CSP antibody disclosed herein has at least one modification, e.g., substitution, relative to the native Fc region of the heavy chain or light chain sequence described herein, and has improved pharmacokinetics properties, e.g., half-life. In certain embodiments, an Fc region of the heavy chain or an Fc region of the light chain of such a variant of an anti-CSP antibody disclosed herein has at least two, three, four, five, or six, or more modifications, e.g., substitutions. In certain embodiments, a variant of an anti-CSP antibody disclosed herein has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g. substitutions, including both heavy and light chains, compared to the parental antibody. In certain non-limiting embodiments, an Fc region of the heavy chain of a variant of an anti-CSP antibody disclosed herein can include an isoleucine at position 250, a tyrosine at position 252, an isoleucine at position 259, a glutamine at position 307, a phenylalanine at position 308, a leucine at position 319, a leucine at position 428, a histidine at position 434, a phenylalanine at position 434, an alanine at position 434, a serine at position 434, a methionine at position 434, or a combination thereof, wherein the numbering is defined by EU index as in Kabat. In certain embodiments, an Fc region of the heavy chain of a variant of an anti-CSP antibody disclosed herein includes a leucine at position 428 and a serine at position 434, wherein the numbering is defined by EU index as in Kabat.

### Antibodies Sequences and Variants Thereof

In certain embodiments, the anti-CSP antibodies disclosed herein comprise a heavy chain variable region and a light variable region. In certain embodiments, the heavy chain variable region comprises an amino acid sequence that is at least about 80%, about 85%, about 90%, about 95%, about 96%, about 97%, about 98%, about 99% identical to the amino acid sequence set forth in SEQ ID Nos. 1-461, as shown in Table 3. In certain embodiments, the light chain variable region comprises an amino acid sequence that is at least about 80%, about 85%, about 90%, about 95%, about 96%, about 97%, about 98%, about 99% identical to the amino acid sequence set forth in SEQ ID Nos. 462-922, as shown in Table 3. In certain embodiments, a) the heavy chain variable region comprises an amino acid sequence that is at least about 80%, about 85%, about 90%, about 95%, about 96%, about 97%, about 98%, about 99% identical to the amino acid sequence set forth in SEQ ID Nos. 1-461; and b) the light chain variable region comprises an amino acid sequence that is at least about 80%, about 85%, about 90%, about 95%, about 96%, about 97%, about 98%, about 99% identical to the amino acid sequence set forth in SEQ ID Nos. 462-922.

In certain embodiments, the heavy chain variable region comprises the amino acid sequence set forth in SEQ ID Nos. 1-461, as shown in Table 3. In certain embodiments, the light chain variable region comprises the amino acid sequence set forth in SEQ ID Nos. 462-922, as shown in Table 3. In certain embodiments, a) the heavy chain variable region comprises the amino acid sequence set forth in SEQ ID Nos. 1-461; and b) the light chain variable region comprises the amino acid sequence set forth in SEQ ID Nos. 462-922.

In certain embodiments, the heavy chain variable region consists of the amino acid sequence set forth in SEQ ID Nos. 1-461, as shown in Table 3. In certain embodiments, the light chain variable region consists of the amino acid sequence set forth in SEQ ID Nos. 462-922, as shown in Table 3. In certain embodiments, a) the heavy chain variable region consists of the amino acid sequence set forth in SEQ ID Nos. 1-461; and b) the light chain variable region consists of the amino acid sequence set forth in SEQ ID Nos. 462-922.

In certain embodiments, the heavy chain variable region comprises a CDR1, a CDR2, and a CDR3 of the heavy chain variable sequence set forth in SEQ ID NOs: 1-461, as shown in Table 3. In certain embodiments, the light chain variable region comprises a CDR1, a CDR2, and a CDR3 of the light chain variable sequence set forth in SEQ ID NOs: 462-922, as shown in Table 3. In certain embodiments, a) the heavy chain variable region comprises a CDR1, a CDR2, and a CDR3 of the heavy chain variable sequence set forth in SEQ ID NOs: 1-461; and b) the light chain variable region comprises a CDR1, a CDR2, and a CDR3 of the light chain variable sequence set forth in SEQ ID NOs: 462-922.

In certain embodiments, the anti-CSP antibodies disclosed herein comprise a heavy chain variable region comprising a CDR1, a CDR2, and a CDR3 and having the amino acid sequence set forth in SEQ ID NOs: 1-461, as shown in Table 3. In certain embodiments, the anti-CSP antibody variant comprises a light chain variable region comprising a CDR1, a CDR2, and a CDR3 and having the amino acid sequence set forth in SEQ ID NOs: 462-922, as shown in Table 3. In certain embodiments, the anti-CSP antibody variant comprises a) a heavy chain variable region comprising a CDR1, a CDR2, and a CDR3 and having the amino acid sequence set forth in SEQ ID NOs: 1-461; and b) a light chain variable region comprising a CDR1, a CDR2, and a CDR3 and having the amino acid sequence set forth in SEQ ID NOs: 462-922. Table 3 is provided below.

In certain embodiments, the anti-CSP antibody variant is designated as indicated in Table 3. For example, but without any limitation, the anti-CSP antibody variant comprising the heavy chain variable region having the amino acid sequence set forth in SEQ ID NO: 1 and the light chain variable region having the amino acid sequence set forth in SEQ ID NO: 462 is designated as “AB-001558”.

TABLE 3

| Ab | | | | |
|---|---|---|---|---|
| Identifier | VH | SEQ ID | VL | SEQ ID |
| AB-001558 | QVQLVESGGGVVQPGRSLRLS | 1 | EIVMTQSPATLSVSPGERATL | 462 |
| | CAASGFTFSSYGMHWVRQAPG | | SCRASQSVSSNLAWYQQKPGQ | |
| | KGLEWVAVIWYDGNNKYYVDS | | APRLLIYGASARATGIPARFS | |
| | VKGRFTISRDNSKNTLYLQMN | | GSGSGTEFTLTISSLQSEDFA | |
| | SLRAEDTAVYYCARDHDADYV | | VYYCQQYNNGRTFGQGTKVEI | |
| | EKWGAFDLWGQGTMVTVSS | | T | |
| AB-001533 | QVQLVESGGGVVQPGRSLRLS | 2 | DIVLTQTPLSSPVTLGQPASI | 463 |
| | CAASGFTFNSYGMHWVRQAPG | | SCRSSQSLVYSDGNTYLNWLQ | |
| | KGLEWVALISFDGTNKFYADS | | QRPGQPPRLLIYKISNRFSGV | |
| | VKGRFTISRDNSKNTLYLQMN | | PDRFSGSGAGTDFTLKISRVE | |
| | SLRAEDTALYHCATDSLFYYD | | AEDVGVYYCMQATQLYTFGQG | |
| | FHLFDYWGQGTLVTVSS | | TKLEIK | |
| AB-001396 | QVQLVESGGGVVQPGRSLRLS | 3 | DIQMTQSPSTLSASVGDRVTI | 464 |
| | CAASGFTFSSYGMHWVRQAPG | | TCRASQSISSWLAWYQQKPGK | |
| | KGLEWVAIIWYDGSHKYYADS | | APKLLIYKASNLGSGVPSRFS | |
| | VKGRFTISRDNSKNTLYLQMN | | GSGSGTEFTLTISSLQPDDFA | |
| | SLRAEDTAVYHCARVYQPTYY | | TYYCQQYNSYWTFGQGTKVEI | |
| | YDSSRGEAFDIWGQGTMVTVS | | K | |
| | S | | | |
| AB-000338 | QVQLVESGGGVVQPGRSLRLS | 4 | AVVMTQSPLSLPVTLGQPASI | 465 |
| | CAASGFSFSTYGMHWVRQAPG | | SCRSSQSLVSSDGNTYLHWFQ | |
| | KGLEWVAIIWYDGSKEFYADS | | QRPGQSPRRLIYKVSNRDSGV | |
| | VKGRFTISRDNSKNTLYLQMN | | PDRFSGSGSGTDFTLKISRVE | |
| | SLRADDTAVYYCVKDDFDSSG | | AEDVGVYYCMQGTHWWTFGQG | |
| | HSYFQHWGPGTLVTVSS | | TKVEIK | |
| AB-001421 | EVQMVESGGRLIKPGGSLRLT | 5 | DIVMTQSPLSLPVTPGEPASI | 466 |
| | CTTSGFSFTNAWMTWVRQAPG | | SCRSSRSLLFRNGDTYLDWYL | |
| | KGPEWVGRIQSESDGGTRDYA | | QKPGQSPQLLIYLASYRASGV | |
| | APVKGRFIISRDDSLNTLYLQ | | PDRFSGSGSGTDFTLKISRVE | |
| | MNSLKTEDTAVYYCITDRDFY | | AEDVGVYYCMQNLQTPATFGQ | |
| | GSTSPSWGQGTLVIVSS | | GTKVEIK | |
| AB-001438 | QVQLVESGGGVVQPGRSLRLS | 6 | QSVLTQPPSVSGAPGQRVTIS | 467 |
| | CAASGFKFISYGMHWVRQAPG | | CTGNSFNIGAGYDVHWYQQLP | |
| | KGLEWVAVIWHDGSIRFYADS | | GTAPKLLIYGNNNRPSGVPDR | |
| | VKGRFTISRDNSNNILYLQMS | | FSGSKSGTSASLAITGLQAED | |
| | SLRAEDTAVYYCATSGYCRGG | | EADYYCQSYDSSLSALVEGGG | |
| | SCDGGVYFDSWGLGTLVTVSS | | TKLTVL | |
| AB-001443 | QVQLVESGGGVVQPGRSLRLS | 7 | DIQMTQSPSTLSASVGDRVTI | 468 |
| | CAASGFTFSTYGMHWVRQAPG | | TCRASQSISSWLAWYQQKPGK | |
| | KGLEWVAVIWYDGSNKYYADS | | APKLLIYKASSLESGVPSRFS | |
| | VKGRFTISRDNSKNTLYLQMN | | GSDFGTEFTLTISSLQPDDFA | |
| | SLRAEDTAVYYCARVRQEPYY | | TYYCQQYNSYWTFGQGTKVEI | |
| | YASRGSALDIWGQGTMVTVSS | | K | |
| AB-001446 | QVQLVQSGAEVKKPGASVKVS | 8 | QPVLTQPPSASASLGASVTLT | 469 |
| | CKASGYTFTSSEINWVRQATG | | CTLSSGYSNYMVDWYQQRPGK | |
| | QGLEWVGWMNPNTHKTIYAQK | | GPRFVMRVGTGGIVGSKGDGI | |
| | LQGRVTMTSDTSKSTAYMELS | | PDRFSVLGSGLNRYLTIKNIQ | |
| | SLRSEDTAVYYCARGGYCIHN | | EEDESDYYCGADYGSGSNFVW | |
| | NCYDVQLDPWGQGTLVIVSS | | VFGGGTKLTVL | |
| AB-001459 | QVQLVEFGGGVVQPGRSLRLS | 9 | DIQMTQSPSTLSTSVGDRVTI | 470 |
| | CSASGFSFRSYGMHWVRQAPG | | TCRASQSINSWLAWYQQKPGK | |
| | KGLEWVAVIWYDGSNYYYTKS | | APKLLIYKASTLEFGVPSRFS | |
| | VEGRFTISRDNSKNMLFLQMN | | GSGSGTEFTLTISSLQPDDFA | |
| | SLRADDTAVYYCARPENSDYD | | TYYCQQYNRYWTFGQGTKVEI | |
| | RGGAFDIWGQGTVVTVSS | | K | |
| AB-001544 | QMQLVESGGGVVQPGRSLRLS | 10 | DIQMTQSPSTLSASVGDRVTI | 471 |
| | CAASGFTFSDYAMHWVRQSPG | | TCRASQSISRWLAWYQQHPGK | |
| | QGLEWVAVISYHSRNKYYPES | | APKLLISGASSLENGVPSRFS | |
| | VKGRFTISRDNSKNTLYLQIN | | GSGSGTDFALTISSLQPDDFA | |
| | SLRTEDTAVYYCARDGYSSSF | | TYYCQHYDSFFVTFGQGTKVE | |
| | FDFWGQGTLVTVSS | | LK | |
| AB-001577 | GVQLVESGGGLVQPGRSLRLS | 11 | EIVLTQSPGTLSLSPGERATL | 472 |
| | CTVSGFTFGDYAMSWVRQAPG | | SCRASHPVTSTYLAWYQQKPG | |
| | KGLEWVGFIRSEANGGKTEYA | | QAPRLLVYGASSRATGIPDRF | |
| | ASVKGRFTISRDDSKSIAYLQ | | SGSGSGTDFTLTVTRLEPEDF | |
| | MRSLTTEDTAVYYCTRVMLGA | | AVYFCQQYGGLPWTFGQGTKV | |
| | SWGLDFWGQGSLVTVSS | | EIK | |
| AB-000391 | EVQLVQSGGGLVQPGGSLRLS | 12 | DVVMTQSPLSLPVTLGQPASI | 473 |
| | CVASGFTLSSYEMNWVRQAPG | | SCRSGQSLLYSDGNTYLYWFQ | |
| | KGLEWVSFISSSGSKKRYADS | | QRPGQSPRRLIYKVSNRDPGV | |
| | VKGRFTVSRDNAKNSLSLQMN | | PERFSGSGSGTDFTLKISRVE | |
| | SLRAEDTAVYYCARDHSTSDR | | AEDVGIYFCLQGTHWPYTFGR | |
| | EYGLDVWGQGATVTVSS | | GTKLEIK | |
| AB-000250 | EVQLVESGGGLVQPGRSLRVS | 13 | DILMTQTPLSLSVTPGQPASI | 474 |
| | CATSGFPFGDHAMSWFRQAPG | | SCKSNESLLHGDGKTYFYWYL | |
| | KGLEWVGFIRSKTYGGAAEYA | | QKPGQSPHLLIYEVSNRFSGV | |
| | ASVKGRFTISRDDSRSIAHLQ | | PDRFSGSGSVTDFTLKISRVE | |
| | MNSLKTEDTGVYYCSRVRTYD | | AEDVGIYYCMQTIDLPWTFGQ | |
| | FRDMDVWGKGTTVTVSS | | GTKVEIK | |
| AB-001529 | QVQLVESGGGVVQPGRSLRLS | 14 | DIQMTQSPSTLSASVGDRVTI | 475 |
| | CVASGFSFRSYGMHWVRQAPG | | TCRASQSISSWLAWYQQKPGK | |
| | KGLEWVAIIWYDGSNKYYAHS | | APKLLIYKASSLESGVPSRFS | |
| | VKGRFTISRDNSKNTLYLQMN | | GSGSGTEFTLTINNLQPDDFA | |
| | SLRAEDTAIYYCAKDGGTETT | | TYYCQQYNSYWTFGQGTKVEI | |
| | WGNAFDIWGQGTMVTVSS | | K | |
| AB-001472 | QVQLVESGGGVVQPGRSLRLS | 15 | DIQMTQSPSTLSASVGDRVTI | 476 |
| | CAASGFTFSSYGMHWVRQAPG | | TCRASQSISRWLAWYQQKPGK | |
| | KGLEWVAVIWYDGSNKYYADS | | APKLLIYDASSLESGVPSRFS | |
| | VKGRFTISRDNSQNTLFLQMN | | GSGSGTEFTLTISSLQPDDFA | |
| | SLRAEDTAVYYCARVGEYSSS | | TYYCQQYNSYWTFGQGTKVEI | |
| | PDWGAAFDIWGQGTMVTVSS | | K | |
| AB-001455 | QVQLVESGGGVVQPGRSLRLS | 16 | DVVMTQSPLSLPVTLGQPASI | 477 |
| | CAASGFTFSNYGMHWVRQAPG | | SCRSSQSLVHSDGNTYLNWFQ | |
| | KGLEWVAIIWYDGSSKYYADS | | QRPGQSPRRLIYKVSNRDSGV | |
| | VRGRFTISRDNSKNTLFLQMN | | PDRFSGSGSGTDFTLKISRVE | |
| | SLRAEDTAVYYCARDDYYSSA | | AEDVGVYYCMQGTHWYTFGQG | |
| | YNHLGYWGQGTLVTVSS | | TKLEIK | |
| AB-001395 | QVQLVESGGGMVQPGRSLRLS | 17 | NFMLTQPHSVSESPGKTVTIS | 478 |
| | CAASGFSFSSYGMHWVRQAPG | | CTRSSGIIASNSVQWYQRRPG | |
| | KGLEWVGVIWYDGGYKLYADS | | HSPTTVIYENDQRPSGVPDRF | |
| | VRGRFTISRDNSKNTLYLQMN | | SGSIDSSSNSASLTISGLRTE | |
| | SLRVEDTAVYYCARPGYCDST | | DEADYYCQSYDWVFGGGTKLT | |
| | NCYAFDYWGQGTLVTVSS | | VL | |
| AB-001478 | QIQLVESGGGVVQPGRSLRLS | 18 | EIVLTQSPATLSLSPGERATL | 479 |
| | CVTSGFTFSNFGMHWVRQAPG | | SCRASHSVGSYLAWYQQKPGQ | |
| | KGLEWVALIWHDGSKKYYADS | | APRLLIYDTSSRAAGVPARFS | |
| | VEGRFTISRDNSKKTLYLQMN | | GSGSGTDFTLTISSLEPEDFA | |
| | SLRAEDTAVYYCARDGGSPDG | | VYYCQQRKNGFTFGPGTKVDI | |
| | SVYNYFEYWGQGTLVTVSS | | K | |
| AB-000327 | QVQLVESGGGVVQPGRSLRLS | 19 | QSMLTQPPSVSGAPGQRVTIS | 480 |
| | CAASRFTFKNYGMHWVRQGPG | | CTGSSSNIGADYDVHWYQQLP | |
| | KGLEWVAVIWYDGSQEYYVDS | | GTAPKLLIYGNNNRPSGVPDR | |
| | VRGRFTISRDNSKDTLYLQMN | | FSGSKSGTSASLAITGLQPED | |
| | SLRAEDTAVYYCVRDGRYRTS | | EAEYYCQSYDSSLSGSFVFGT | |
| | HQTSTGFFDLWGQGTLVTVSS | | GTKVTVL | |
| AB-001387 | QVQLVESGGGVVQPGRSLRLS | 20 | QSVLTQPPSVSGAPGQRVTIS | 481 |
| | CAASGFTFSNYGMHWVRQAPG | | CTGSSSNIGAGYDVHWYQQLP | |
| | KGLEWVAVIWYDGSNKFYADS | | GTAPKLLIYGNSNRPSGVPDR | |
| | VKGRFTSSRDNSKNTLYLQMN | | FSGSKSGTSASLAITGLQAED | |
| | SLRAEDTAVYYCARDGRELTT | | EADYYCQSYDSSLSALVFGGG | |
| | VTTAYFDYWGQGTLVTVSS | | TKLTVL | |
| AB-001554 | QVQLVESGGGVVQPGRSLRLS | 21 | DVVMTQSPLSLPVTLGQPASI | 482 |
| | CAASGFTFSSYGMHWVRQAPG | | SCRSSQSLVYSDGNTYLNWFQ | |
| | KGLEWVAVIWYDGSNKYHADS | | QRPGQSPRRLIYKVSNRDSGV | |
| | VKGRFTISRDNSKNTLYLQMN | | PDRFSGSGSGTDFTLKISRVE | |
| | NLRSEDTAVYYCAKDETLWFG | | AEDVGIYYCMQGTHWPRTFGG | |
| | ELSDGFDIWGQGTMVTVSS | | GTKVEIK | |
| AB-001545 | QVQLVESGGGVVQPGRSLRLS | 22 | EIVLTQSPATLSLSPGERATL | 483 |
| | CAASGFTFSSYGMHWVRQAPG | | SCRASQSVSSYLAWYQQKPGQ | |
| | KGLEWVAVIWYDGSNKYYADS | | APRLLIYDASNRATGIPARFS | |
| | VKGRFTISRDNSKNTLYLQMN | | GSGSGTDFTLTISSLEPEDFA | |
| | SLRAEDTAVYYCARDLDYSNH | | VYYCQQRSNWFTFGQGTRLEI | |
| | NWFDPWGQGTLVTVSS | | K | |
| AB-001482 | QVQLVESGGGVVQPGRSLRLS | 23 | DIQMTQSPSTLSASVGDRVTI | 484 |
| | CAASGFTFRDYGMHWVRQVPG | | TCRASQSIISSLAWYQQKPGK | |
| | KGLEWVAIIWHDGSKKYYADS | | APKLLIYKASSLENGVPSRFS | |
| | VKGRFTISRDNFKNTLYLQMN | | GSGSGTEFTLTISSLQPDDFA | |
| | SLRAEDTAVYYCAKVFDHAES | | TYYCQQYNSYWTFGQGTKVEI | |
| | SYTFDIWGQGTMVTVSS | | I | |
| AB-000314 | QVQLVESGGGVVQPGRSLRLS | 24 | YIQMTQSPSTLSASVGDRVTI | 485 |
| | CAASGFPFSSYGMHWLRQAPG | | TCRASHNISSWLAWYQQKPGK | |
| | KGLEWVAVIWYDGSNYYYADS | | APNLLIYKASSLESEVPSRFS | |
| | VKRRFTISRDNSKNTLYLQMN | | GSGSGTEFTLTISSLQPDDFA | |
| | SLRAEDTAVYYCARVGMGDYL | | TYYCQQYKNYWTFGQGTKVEI | |
| | DSSGYYDYWGQGTLVTVSS | | K | |
| AB-000349 | EVQLVESGGGLVQPGGSLRLS | 25 | DTVMTQTPLFLSVTPGQPASI | 486 |
| | CAASGFTFNNYWMSWVRQTPG | | SCKSSQSLLHSDGRTYLYWYL | |
| | MGLEWVANINQDGSKKDYVDS | | QKPGQSPQLLIYEVSNRFSGV | |
| | VEGRFTISRDNAKNSLYLLMN | | PERFSGSGSGTDFTLRISRVE | |
| | SLRADDTAVYYCARYLNAWGQ | | AEDVGIYYCMQSIYLPITFGG | |
| | GILVTVSS | | GTKMEIK | |
| AB-001480 | QVQLVESGGGVVQPGRSLRLS | 26 | DIQMTQSPSTLSASVGDRVTI | 487 |
| | CAASGFTFSFYGMHWVRQAPG | | TCRASQSISSWLAWYQQKPGK | |
| | KGLEWVAVIWYDGSNKYYADS | | APKLLIYRASSLESGVPPRES | |
| | VRGRFTISRDNSKNTLYLQMN | | GSGSGTEFTLTISSLQPDDFG | |
| | SLRAEDTAVYYCARVGNDYDD | | IYYCQQYNSRWTFGQGTKVEI | |
| | HGSALDMWGQGTMVTVSS | | K | |
| AB-001428 | QVQLVESGGGVVQPGRSLRLS | 27 | DIQMTQSPSTLSASVGDRVTI | 488 |
| | CVVSGFTFSSYGMHWVRQAPG | | TCRPSQSISNWLAWYQQKPGK | |
| | KGLEWVAIIWYDGSKKYYADS | | APKLLIYKASSLESGVPSRFS | |
| | VKGRFTISRDNSKNTLYLQMN | | GSGSGTEFTLTISGLQPDDFA | |
| | SLRAEDTAVYYCARPGDSSGS | | TYYCQQYNTYWTFGQGTKVEI | |
| | HGEAFDIWGQGTMVTVSS | | K | |
| AB-001495 | QVQLVESGGGVVQPGRSLRLS | 28 | QSVLTQPPSVSGAPGQRVTIS | 489 |
| | CAASGFTFSSYGMHWVRQAPG | | CTGSSSNIGAGYDVHWYQQLP | |
| | KGLEWVAVIWYDGSYKYYADS | | GTAPKLLIYGNSNRPSGVPDR | |
| | VKGRFAVSRDNSKNTLYLQMN | | FSGSKSGTSASLAITGLQAED | |
| | SLRTEDTAVYYCAKEGRTVEV | | EADYYCQSYDSSLSGFVFGTG | |
| | AATGWFDPWGQGTLVTVSS | | TKVTVL | |
| AB-000373 | QVLLVESGGGVVQPGGSLRLS | 29 | DAVMTQTPLSLSVTPGQPASI | 490 |
| | CTASGFTFKNYGMHWVRQAPG | | SCKSGQSLLHSDGKTYLYWYL | |
| | KGLEWVAVIWFDGSNEFYADS | | QKPGQSPQLLIYEVSSRFSGV | |
| | VEGRFTISRDNSKNTLYLQTN | | PDRFSGSGSGTDFTLKISRVE | |
| | SLRAEDTAVYYCARSYTSSWL | | AEDVGVYYCMRGIHPRTFGQG | |
| | GFDFWGQGTLVTVSS | | TKVEIK | |
| AB-001458 | EVQLVESGGGLVQPGRSLRLS | 30 | EIVMTQSPATLSVSPGERATL | 491 |
| | CTASGFTFGDYAMSWFRQAPG | | SCRASQSVGSNLAWYQQKPGQ | |
| | KGLEWVSFIRSKTYGGSTEYA | | APRLLIYGASTRATGLPARFS | |
| | ASVKGRFTISRDDSKSIAYLQ | | GSGSATEFTLTISSLQSEDFA | |
| | MHSLKTEDTAFYYCTRVSAAA | | VYYCQQYNTWWTFGQGTKVEI | |
| | GVGDYWGQGTLVTVSS | | K | |
| AB-001511 | QVQLVESGGGVVQPGRSLRLS | 31 | DVVMTQSPLSLPVTLGQPASI | 492 |
| | CAASGFTFSSYGMHWVRQAPG | | SCRSSQSLVYSDGNTYLNWFQ | |
| | KGLEWVAIIWYDGSNKYYADS | | QRPGQSPRRLIYKVSNRDSGV | |
| | VKGRFTISRDNSKNTLYLQMN | | PDRFSGSGSGTDFTLKISRVE | |
| | SLRAEDTAVYYCARDTDDYYD | | AEDVGVYYCMQGTHLYTFGQG | |
| | AHTMDVWGQGTTVTVSS | | TKLEIK | |
| AB-001468 | QVQLVESGGGVVQPGRSLRLS | 32 | DIQMTQSPSTLSASVGDRVTI | 493 |
| | CAASGFTFSSYGMHWVRQAPG | | TCRASQSIGSWLAWYQQKPGK | |
| | KGLEWVAIIWYDGSNKYYADS | | APKLLIHEASSSESGVPSRFS | |
| | VKGRFTISRDKSKNTLYLQMN | | GSGSGTEFTLTISSLQPDDFA | |
| | SLRAEDTAVYYCAKVSDSSDY | | TYYCQQYNSYWTFGQGTKVEI | |
| | SHSGAFDIWGQGTMVTVSS | | K | |
| AB-001551 | EVQLVESGGGLVQPGRSLRLS | 33 | EIVMTQSPATLSVSPGERATL | 494 |
| | CTASGFTFGDYAMSWFRQAPG | | SCRASQSISSNLAWYQQKPGQ | |
| | KGLEWVGFIRSKANGGTGEYA | | APRLLIYGASTRATGIPARFS | |
| | ASVKGRFTISRDDSKNIAYLQ | | GSGSGTEFTLTISSLQSEDFA | |
| | MDSLKTEDTAVYYCTRVLVVV | | VYYCQQYHDWWTFGQGTKVEI | |
| | AAGDYWGQGTLVTVSS | | K | |
| AB-000376 | QVQLVESGGGVVQPGRSLRLS | 34 | DIQMTQSPSTLSASVGDRVTI | 495 |
| | CTASGFTFSNYGMHWVRQAPG | | TCRASHSISNWLAWYQQKPGK | |
| | RALEWVAIIWHDGSNKYYADS | | APRLLIYKASSLNSGVPSRFS | |
| | VKGRFTISRDNSKNTLYLQMN | | GSASGTEFTLTISSLQPDDFA | |
| | NVRVEDTAVYHCAKVGGFGEL | | TYFCQQYNAYWTFGQGTKVEI | |
| | YEAFDFWGQGTLVTVSS | | K | |
| AB-000394 | QAQPVESGGGVVQPGKSLRLS | 35 | EIVLTQSPATLSLSPGERATL | 496 |
| | CAASGFTFRNFGMHWVRQAPG | | SCRASQSISDYLAWYQQKPGQ | |
| | KGLEWVAVIWHDGSNQFYADS | | APRLLIYDASFRATGIPARFS | |
| | VEGRFTISRDNSKNTLYLQMD | | GSGSGTDFTLTISSLEPEDFA | |
| | SLRVEDTAMYYCARDRGYSGS | | VYYCQQRSIGFTFGPGTKVDI | |
| | WGYFDHWGQGTLVTVSS | | K | |
| AB-000222 | QVQLVESGGGVVQPGRSLRLS | 36 | QFVLTQPPSVSGAPGQRVTIS | 497 |
| | CAASGFTFSNFGMHWVRQAPG | | CTGSSFNIGAGYDVHWYQQLP | |
| | KGLEWVAVIWYDGSKKYYADE | | EKAPKLLIYGSTNRASEVPDR | |
| | VKGRFTISRDNSKYMLYLQMT | | FSGSKSGTSASLAITGLQPED | |
| | SLGAEDTAIYYCARDIRGAYR | | EADYYCQSYDSSLSGYVFGTG | |
| | VTDGGFDYWGQGALVTVSS | | TKVTVL | |
| AB-001510 | QVQLVESGGGVVQPGRSLRLS | 37 | DIQMTQSPSTLSASVGDRVTI | 498 |
| | CAASGFTFRNNGMHWVRQAPG | | TCRASQSISSWLAWYQQKPAE | |
| | KGLEWVAVIWHDGSNKYYADA | | APKLLIYEASTLESGVPSRFS | |
| | VKDRFTISRDNSKNTLYLQMN | | GSGSGTEFTLTISSLQPDDFA | |
| | SLRAEDTAVYYCVKVAFCTGD | | TYYCQQYNTYWTFGQGTKLEI | |
| | CYPPMDVWGKGTTVTVSS | | K | |
| AB-001388 | QAQLVESGGGVVQPGRSLRLS | 38 | EIVLTQSPGTLSLSPGERATL | 499 |
| | CAASGFTFSSYGMHWVRQAPG | | SCRASQSVSGNYLAWYQRKPG | |
| | KGLEWVAVIWHDGSNQYYADS | | QAFRLLIYGASSRATGIPDRE | |
| | VKGRFTISRDNSKNTLYLQMN | | SGSGSGTDFTLTISRLEPEDF | |
| | SLRAEDTAVYYCAKVGDLLAL | | AVYFCQQYGRLWTFGQGTKVE | |
| | DYWGQGTLVTVSS | | IR | |
| AB-000315 | QVQLVESGGGVVQPGRSLRLS | 39 | DIQMTQSPSTLSASVGDRVTI | 500 |
| | CAASGFMFKNYGMHWVRQAPG | | TCRASHSISSWLAWYQQKPGK | |
| | EGLEWVTVIWHDGSNEYYADS | | APKLLIYKASTLESGVPSRFS | |
| | VKGRFTVSRDNSKNTLYLQMN | | GSGSGTEFTLTISSLQPDDFA | |
| | SLRAEDTAVYYCARVGDGTTK | | TYYCQQYDYYWTFGQGTKVEI | |
| | YDSSGFLDCWGQGTLVTVSS | | K | |
| AB-001578 | QVQLVESGGGVVQPGRSLRLS | 40 | EIVLTQSPATLSLSPGERATL | 501 |
| | CAASGFSFSNYGMHWVRQAPG | | SCRASQGVSSYLAWYQQKPGQ | |
| | KGLEWVAIIWYDGSNKYYADS | | APRLLIYDASNRATGIPARFS | |
| | VKGRFTISRDNSKNTLYLQMN | | GSGSGTDFTLTISSLEPEDFA | |
| | SLRAEDTAVYYCARDGTIWDD | | VYYCQQRSNWPPTFGPGTKVD | |
| | SRGYILDYWGQGTLVTVSS | | IK | |
| AB-001517 | QVQLVESGGGVVQPGRSLRLS | 41 | DIVMTQTPLSLSVSPGQPASI | 502 |
| | CAASGFTFSNYGLHWVRQAPG | | SCKSSQSLLHSDGKTYLHWYM | |
| | KGLEWVALIRYDGNNKFYADS | | QKPGQSPQVLIYEVSSRFSGV | |
| | LKGRFTISRDNSKDTLYLQMN | | PDRFSGSGSGTDFTLEISRVE | |
| | SLRVEDTAVYYCAKVGSGNYG | | AEDVGVYFCMQSIELWTFGQG | |
| | NYMDVWGKGTTVTVST | | TKVEIK | |
| AB-000325 | QVQLVESGGGVVQPGRSLRLS | 42 | EIVMTQSPATLSVSPGERATL | 503 |
| | CAASRFPFKNYGMHWVRQAPG | | SCRASQSVGSKLAWYQHKPGQ | |
| | KGLEWVALIWFDGSNKFYVDS | | APRLLIYGASTRATGIPARFS | |
| | VEGRFTISRDNSNNMLYLEMN | | GSGSGTEFTLTVSSLQSEDFA | |
| | SLRADDTAVYYCARAGRYDDS | | VYYCQQYNHWYTFGQGTKLEI | |
| | GYLDYWGQGTLVTVSS | | K | |
| AB-001513 | QVQLVESGGGVVQPGRSLRLS | 43 | QSVLTQPPSVSGAPGQRVTIS | 504 |
| | CAASGFTFSSYGMHWVRQAPG | | CTGSSSNIGAGYDVHWYQQLP | |
| | KGLEWVAVIWYDGSNKFYADS | | GTAPKLLIYDNSNRPSGVPDR | |
| | VKGRFTVSRDNSKNTLYLQMN | | FSGSKSGTSASLAITGLQAED | |
| | SLRAEDTTVYYCARDGGVTTR | | EADYYCQSYDSSLSALVFGGG | |
| | YFDLWGRGTLVTVSS | | TKLTVL | |
| AB-001492 | QEQLVESGGGVVQPGRSLRLS | 44 | DIQLTQSPSFLSASVGDRVTI | 505 |
| | CAASGFTFSTYAIHWVRQAPG | | TCRASQGISSYLAWYQQKPGK | |
| | KGLEWVAVISYDGSSTWFSNS | | APKLLIYAASTLQSGVPSRFS | |
| | VEGRFTFSRDNSKNTVFLQMN | | GSGSGTEFTLTISSLQPEDFA | |
| | SLSAEDTAVYYCARDGNDYPA | | TYYCQQHNRYPPTFGGGTKVE | |
| | YYYYMDVWGKGTTVTVSS | | IK | |
| AB-001557 | QVQLVESGGGVVQPGRSLRLS | 45 | QSVLTQPPSVSGAPGQRVTIS | 506 |
| | CAASGFTFSTYGMHWVRQAPG | | CTGSSSNIGAGYDVHWYQQLP | |
| | KGPEWVAVIWYDGSNKYYRDS | | GTAPKLLIYGNTNRPSGVPDR | |
| | VKGRFSISRDNSKNTLYLQMN | | FSGSKSGTSASLAIAGLQADD | |
| | SLRAEDTAVYYCAKDGRPLKG | | EADYYCQSYDSSLSVFVFGTG | |
| | GVTTGWFDPWGQGTLVTVSS | | TKVTVL | |
| AB-001462 | QVQLVESGGGVVQPGRSLRLS | 46 | DIQMTQSPSSLSASVGDRVTI | 507 |
| | CAASGFSFSSYGMHWVRQAPG | | TCRASQGISNYLAWFQQKPGK | |
| | KGLEWVAVIWYDGSNKYYADS | | APKSLIYAASSLQSGVPSKFS | |
| | VKGRFTISRDNSKNTLYLQMN | | GSGSGTDFTLTISSLQPEDFA | |
| | SLRAEDTAVYYCATAAQGYCS | | TYYCQQYNSYPPTFGQGTKVE | |
| | GGSCYGHTFDYWGQGTLVTVS | | IK | |
| | S | | | |
| AB-001508 | QVQLVESGGGVVQPGRSLRLS | 47 | DIVMTQSPDSLAVSLGERATI | 508 |
| | CAASGFTFSSYGMHWVRQAPG | | NCKSSQSVLYSSNNKNYLAWY | |
| | KGLEWVAVIWYDGSKKYYADS | | QQKPGQPPKLLIYWASTRESG | |
| | VKGRFTISRDNSKNTVYLHMN | | VPDRFSGSGSGTDFTLTISSL | |
| | SLRVEDTAVYYCARPEAVAGT | | QAEDVAVYYCQQYYSILPLTF | |
| | GGWFDHWGQGNRVTVSS | | GGGTKVEIK | |
| AB-001465 | EVQLVQSGAEVKKPGESLKIS | 48 | DVVMTQSPLSLPVTLGQPASI | 509 |
| | CKGSGYSFTSYWIGWVRQMPG | | SCRSSQSLVHSDGNTYLNWFQ | |
| | KGLEWMGIIYPGDSDTRYSPS | | QRPGQSPRRLIYKVSNRDSGV | |
| | FQGQVTISADKSISTAYLQWS | | PDRFSGSGSGTDFTLKISRVE | |
| | SLKASDTAMYYCARHYSDTSD | | AEDVGVYYCMQGTHWPYTFGQ | |
| | YYYDYWGQGTLVTVSS | | GTKLEIK | |
| AB-000320 | QVQLVESGGGVVEPGGSLRLS | 49 | DIQMTQSPSSLSASVGDRVTI | 510 |
| | CAASGFTFSNYGMHWVRQAPG | | TCQASQDISNYLNWYQQRPGK | |
| | KGPEWVAVIWHDGSKKYYEDS | | APKVLIYDASNLAVGVPSRFS | |
| | VEGRFTISRDNSKNTLYLKMN | | GSGSGTDFSFTISSLQPEDFA | |
| | SLRVEDTAVYYCAKDRGGDRG | | TYFCQQYHTSFTFGPGTKVDI | |
| | NFGHYWGQGTLVTVSS | | K | |
| AB-001500 | EVQLVESGGGLVQPGRSLRLS | 50 | QSALTQPASVSGSPGQSITIS | 511 |
| | CTASGFTFGDYAMSWVRQAPG | | CTGTSSDVGGYNYVSWYQQHP | |
| | KGLEWVGFIRRKAHGGTTEYA | | GKAPKLMIYDVSNRPSGVSNR | |
| | ASVKGRFTISRDDSKSIAYLQ | | FSGSKSGNTASLTISGLQAED | |
| | MNSLKTEDTAVYYCTRVEPTV | | EADYYCSSYTSSSTHVLFGGG | |
| | VTPVFDYWGQGTLVTVSS | | TKLTVL | |
| AB-001516 | QEQLVESGGAVVQPGGSLRLA | 51 | QSVLTQPPSVSGAPGQRVTIA | 512 |
| | CVGTGFSFSSYGIHWVRQAPG | | CSGSSSNIGAGYDVHWYQQLP | |
| | KGLEWVALISYDGSNKYYGES | | GTAPKLLIYGNTNRPSGVPDR | |
| | VRGRFTISRDNSKDTVYLQMK | | FSGSKSGTSASLAISGLQAED | |
| | SLRGDDTAVYYCATAYYNFWH | | EADYYCQSYDTSRGGLLFGGG | |
| | SYYMLPTKVLGMDVWGQGTTV | | TKLTVL | |
| | TVSS | | | |
| AB-001475 | EVQLVQSGAEVRKPGTTVKIS | 52 | QPVLTQPPSASASLGASVTIT | 513 |
| | CDVSGYTFTDYYIYWVQQAPG | | CTLSRGYNNYKVDWYQQRPGK | |
| | KGLEWMGLVDLENGETKYAEK | | GPRFVMRVGTGGIVGSKGDGI | |
| | FQGRVTITADTSTDIAHMELS | | PDRFSVLGSGLNRYLTIKNIR | |
| | SLRSEDTAIYYCATDSSCGDE | | EEDEGDYHCGTDHASGNNFVY | |
| | CNVPGGAFDIWGQGTIITVSS | | VFGTGTKVIVL | |
| AB-001565 | QVHLVQSGAEVKKPGASVKVS | 53 | DIQMTQSPSSLSASVGDRVTI | 514 |
| | CKTSGYSFTGYYMHWVRQAPG | | TCRASQDISNYLNWYQQKPGK | |
| | QGLEWMGWINPHSGGTKDAPK | | APKLLIYAASNLQSGVPSRFS | |
| | FQGRVTLTRDTSINTSYMELD | | GSGFGTEFSLTISNIQPEDVA | |
| | SLTSDDTAVYFCARSEQVVLL | | TYCCQQSHSLWTFGQGTKVEI | |
| | GGFDFWGQGTLVTVSS | | K | |
| AB-000393 | EVQLVESGGGLVQPGRSLRLS | 54 | DIVMTQTPLSLSVTPGQPASI | 515 |
| | CTTSGFSFGDYAMNWVRQAPG | | SCSSSLSLLRSDGKTYLYWYL | |
| | KGLEWVGFIRSNAYGGTAEYA | | QKPGQPPQLLIYEVSNRFSGV | |
| | ASVKGRFTISRDDPNSIAYLQ | | PDRFSGSGSGTDFTLKISRVE | |
| | MNSLRTEDTAVYYCTRLEAGV | | AEDVGVYYCMQSVELPYTFGQ | |
| | KRGMDVWGQGTTVTVSS | | GTKLEIK | |
| AB-007104 | QVQLVESGGGVVQPGKSLRLS | 55 | VVVMTQSPLSLPVTLGQPASI | 516 |
| | CIASGFPFSTYGMHWVRQAPG | | SCKSSQSLVHSDGNTYLHWFQ | |
| | KGLEWVAIIWHDGSKEFYGDS | | QRPGQSPRRLIYKVSNRDSGV | |
| | VKGRFTISRDNSKNTLYLQMN | | PDRFSGSGSGTDFTLKISRVE | |
| | SLTAEDTAMYYCVKDDFDSSG | | AEDVGVYYCMQGTHWWTFGQG | |
| | YSYFQHWGQGTLVTVSS | | TKVEIK | |
| AB-007088 | QVQLVESGGGVVQPGRSLRLS | 56 | GVQMTQSPSTLSASVGDRVTL | 517 |
| | CAASGFAFNTYGMHWVRQTPG | | TCRASQSISSWLAWYQQKPGK | |
| | KGLEWVAIIWYDGSQKYYADS | | APKLLIYDASSLESGVPSRFS | |
| | VQGRFIISRDNHKNTLSLQMN | | GSGSGTEFTLTISSLQPDDFA | |
| | GLRAEDTAVYFCVRVRESVGP | | TYYCQQYNSYSFWTFGQGTKV | |
| | HGSAFDLWGQGTMVIVSS | | EIK | |
| AB-007103 | QVQLVESGGGVVQPGRSLRLS | 57 | DVVLTQSPLSLPVTLGQPASI | 518 |
| | CAASGFTFSSFGMHWVRQAPG | | SCRSSQSLVHSDGNTYLNWFQ | |
| | KGLEWVAIIWYDGNNKYYADS | | QRPGQSPRRLIYKVSKRDYGV | |
| | VKGRFTISRDNSKNTLYLQMN | | PERFSGSGSGTDFTLKISRVE | |
| | SLRDDDTAVYYCAKDDYDSGG | | AEDVGVYYCMQGTHWWTFGQG | |
| | YNQFESWGQGTLVIVSS | | TKVEIK | |
| AB-007114 | EVQLVESGGGLVQPGRSLRLS | 58 | QSVLTQPPSVSGAPGQRVTIS | 519 |
| | CKASGFNFGDHAVSWVRQAPG | | CTGGSSNIGAGYDIYWYQQLP | |
| | KGLEWVGFIRSKVYGGTTKYA | | GTAPKLLMYAYTNRPSGVPDR | |
| | ASVKGSFTISRDDSKSISYLE | | FSGSKSGTSASLAITGLQAED | |
| | MNSLKTEDTAVYYCTRVQLDY | | EADYYCQSYDTSLDGWVFGGG | |
| | GPGYYYYGMDVWGQGTKVTVS | | TKLTVL | |
| | S | | | |
| AB-007176 | QVQLVESGGGVVQPGRSLRLS | 59 | QSVLTQPPSVSGAPGQRVTVS | 520 |
| | CAASGFTFKNRGMHWVRQAPG | | CTGSSSNIGAGYDVHWYQQLP | |
| | KGLEWVAVIWHDGSKEFYADS | | GTAPKLLIYGNNNRPSGVPER | |
| | VEGRFTVSRDNSKNTLYLQMN | | FSGSRSGTSASLAITGLQADD | |
| | SLRAEDTAIYYCVRAAYYDTS | | EGDYYCQSYDRRLMNSWVEGG | |
| | GYGDYWGQGALVTVSS | | GTKLTVL | |
| AB-007131 | QVQLVESGGGMVQPGRSLRLS | 60 | EIVMTQSPATLYVSPGERATL | 521 |
| | CVASQFTFNNYGMHWVRQAPG | | SCRASQSVGRDLAWYQQKPGQ | |
| | RGLEWLALIWYDGSNKFYADS | | APRLLIYGASTRTTGVPARES | |
| | VEGRFTISRDNSKNTLYLQMS | | GSGSGTEFTLTLNSLQSEDFA | |
| | SLRADDTAVYYCARAGKYDNS | | VYYCQQYNKWYTFGQGTKLAI | |
| | GYLDYWGQGTLVTVSS | | K | |
| AB-007101 | QMQLVESGGGVVQPGRSLRLS | 61 | EIVLTQSPATLSLSPGERATL | 522 |
| | CAASGFSFNTYGMHWVRQAPG | | SCRASQSVSSSVAWYQQKPGQ | |
| | KGLEWVAVIWHDGSKKFYRDS | | APRLLIYDASNRATDVPARFS | |
| | VEGRFTVSRDNSKDTLYLQMN | | GSGSGTDFTLTISSLEPEDFA | |
| | SLRVEDTAVYYCARDFSYSSH | | VYYCQQRSSWFTFGQGTRLDI | |
| | NYFHPWGQGTLVTVSS | | K | |
| AB-007161 | EVQLVESGGGLVQPGRSLRLS | 62 | DTVLTQTPLSLSVIPGQPASM | 523 |
| | CATSGFIFGDYAMSWFRQAPG | | SCKSSQSLLDNDGKTYLYWYL | |
| | KGLEWVGFIRSKGYGGSTEYA | | QKPGQSPQLLIYEVSNRFSGV | |
| | ASVKGRFTISRDDSKSVAYLQ | | PDRFSGSGSGTDFTLKISRVE | |
| | MNSLKTEDTAVYYCSKIGIVV | | AEDVGIYYCMQRIQLPWTFGQ | |
| | PSAVYWGQGTLVTVSS | | GTKVEIK | |
| AB-007121 | QVQLVESGGGVVQPGRSLRLS | 63 | NVVLTQTPLSSPVTLGQPASI | 524 |
| | CAASGFTFNNYGMHWVRQAPG | | SCRSSQSLVYSDGNTYLNWLQ | |
| | KGLQWVALISYDGSNKFYADS | | QRPGQPPRLLIYKVSNRFTGV | |
| | VEGRFTISRDSADNTLYLQMD | | PDRFSGSGAGTDFTLNISRVE | |
| | SLRPEDTAVYYCATDSLNYYD | | AEDVGVYFCMQATQLYTFGQG | |
| | FHRFDNWGQGTLVIVSS | | TKLEIK | |
| AB-007173 | QVQLVESGGGVVQPGRSLRLS | 64 | QSVLTQPPSVSGAPGQRVTIS | 525 |
| | CAASGFTFYRYGMHWVRQAPG | | CTGSSSNIGADYDVHWYQQLP | |
| | KGLEWVAVLWHDGSNKYYADS | | GTAPKLLIYGNNNRPSGVPDR | |
| | LKGRFTISRDNSKSTLYLQMN | | FSGSKSGTSASLAITGLQAED | |
| | SLTAEDTAVYYCARAAYHDTS | | EADYYCQSYDSSLSSSWVFGG | |
| | GYGDYWGQGTLVTVSS | | GTKVTVL | |
| AB-007154 | QLQLVESGGGVVQPGRSLRLS | 65 | DIQMTQSPPTLSASVGDRVTI | 526 |
| | CAASGFTFSSYGMHWVRQAPG | | TCRASQSISTWLAWYQQKPGK | |
| | KGLEWVAVIWHDGSTKYHAES | | APKLLIYKASTLKSGVPSRFS | |
| | VEGRFTISRDNSRNTLYLQMN | | GSGSGTEFTLTVSSLQPDDFA | |
| | SLRAEDTAVYYCARVGMGGQY | | TYYCQQYSYFWTFGQGTKVEI | |
| | DSSGYYDYWGQGTLVTVSS | | K | |
| AB-007083 | QVQLVESGGGAVQPGRSLRLS | 66 | GVQMTQSPSTLSASVGDRVTL | 527 |
| | CVASGFGFRNYGMHWIRQAPG | | TCRASQNINNWLAWYQQKPGK | |
| | RGLEWVAIIWYDGSQKYYSDS | | APKLLIYEASILESGVPSRFS | |
| | VEGRFTISRDNSKNTLYLQMN | | GSGSGTEFTLTISSLQPDDFA | |
| | SLRVEDTAIYYCVRVRYSSSR | | TYYCQQYNDYSFWTFGQGTKV | |
| | HGSAFDLWGQGTTVIVSS | | DIK | |
| AB-007163 | QVHLLESGGTVVQPGGSLRLS | 67 | QSVLTQPPSISGAPGQRVTIS | 528 |
| | CEASGFIFSDYGMHWVRQVPG | | CTGAGSNIGAGYEVHWYQQLP | |
| | KGLEWVAVIWFDGSQKYYGDS | | GTAPKLLMFADNNRPSGVPDR | |
| | VKGRFTVSRDNSKNTLYLQMN | | FSGSKSGTSASLAITGLQAED | |
| | TLRADDTGIYYCATDIRGAYK | | EADYYCQSYDISLKNFVFGTG | |
| | VTDGGFDQWGHGTLVTVSP | | TTVTVL | |
| AB-007087 | QAQLVESGGGVVQPGRSLRLS | 68 | DIQLTQSPSTLSASVGDRVTI | 529 |
| | CAASGFTFRNYGMHWVRQAPG | | TCRASQSVNRWLAWYQQKPGK | |
| | KGLDWVAIIWYDGSQTYYADS | | APKLLIYEASTLEIGVPSRFS | |
| | VQGRFTISRDNSKDTLYLQMN | | GSGSGTEFTLTISSLQPDDFA | |
| | NLRAEDTAVYYCARVRYSSSQ | | TYHCQQYNSYSFWTFGQGTKV | |
| | HGSAFDLWGQGTMVTVSS | | EIK | |
| AB-007178 | QVQLVESGGGVVQPGRSRRLS | 69 | EVVMTQSPATLSVSPGERATL | 530 |
| | CAASGFYFNTYGMHWVRQAPG | | SCRASQGVSSNLAWYQQKPGQ | |
| | KGLEWVALIWFDGSKKFYADS | | PPRLLIYGASTRATGIPARFS | |
| | VKGRFTISRDNSKDTLYLQMN | | GSGSGTEFTLTISSLQSEDFA | |
| | SLRAEDTAVYYCARDGAAYDD | | VYYCQQYSNWWTFGQGTKVEI | |
| | SNGGAFDIWGQGTLVTVSS | | K | |
| AB-007120 | QVQLVESGGGVVQPGRSLRLS | 70 | DIVLTQTPLSSPVALGQPASI | 531 |
| | CAASGLIFRSSGMHWVRQAPG | | SCRSSQSLVDSDGNTYLNWLQ | |
| | KGLEWVSLISYDGSNKFYADS | | QRPGQPPRLLIYKISNRFSGV | |
| | VKGRFTISRDNSKNTLYLQMN | | PDRFSGSGAGTDFTLKISRVE | |
| | SLRADDTAVYYCATDSLSYYD | | AEDVAVYYCMQATQLYTFGQG | |
| | YHLFDYWGQGALVTVSS | | TKLEIK | |
| AB-007110 | EVQLVESGGGLVQPGRSLRLS | 71 | QSVLTQPPSVSGAPGQRVTIS | 532 |
| | CIASGFTFGDHAMSWVRQAPG | | CTGSSSNIGAGYDVNWYQQFP | |
| | RGLEWLGFIRSKTYGGTPKYA | | GTAPKLLIYAYTNRPSGVPDR | |
| | ASVKGRITISRDDSKSMVYLQ | | VSGSKSGTSASLAITGLQAED | |
| | MNSLKTDDTAVYYCTRVQLDQ | | EADYYCQTYDSSVDGWVFGGG | |
| | GPGYYYYGMDVWGQGTTVTVS | | TKLTVL | |
| | S | | | |
| AB-007096 | QEQLVESGGGVVQPGGSLRLS | 72 | EIVLTQSPATLSLSPGERATL | 533 |
| | CAASGFSFGSYGMHWVRQAPG | | SCRASQSVGRSLAWYQQKPGQ | |
| | KGLEWVAVIWYDGSQKFHADS | | SPRLLIYEASNRATGIPARES | |
| | LKGRFTISRDNVKNTLYLQMN | | GSGSGADFTLTISSLEPEDFA | |
| | SLRGEDTAVYYCARDLDYYNH | | VYYCQQRSNWFTFGQGTRLEI | |
| | NYFDPWGQGTLVTVSS | | K | |
| AB-007123 | QVQLVESGGGVVQPGRSLRLS | 73 | DIVLTQTPLLSPVTLGQPASI | 534 |
| | CAASGFTFRNSGMHWVRQAPG | | SCRSSQSLVYSDGNTYLSWLQ | |
| | KGLEWVALITHDGSNEFYADS | | QRPGQPPRLLIYKISNRFSGV | |
| | VKGRFTISRDNSKNTLFLQMN | | PDRFSGSGAGTSFTLKISRVE | |
| | SLRADDTAMYYCATDSLHYFD | | AEDVGVYYCMQATQLYTFGQG | |
| | YHLFDYWGQGTLVTVSS | | TKLEIK | |
| AB-007182 | QVYLVESGGGVVQPGRSLRLS | 74 | DIQMTQSPSTLSASVGDRVTI | 535 |
| | CAASGFIFRSYGMHWVRQAPG | | TCRASQSISSWLAWYQQKPGK | |
| | KALEWVAVIWYDGSNKFYSDS | | APKLLIYRASILQTGVPSRFS | |
| | VKGRFTISRDNSKNTLYLEMN | | GSGSGTEFTLTISSLQPDDFA | |
| | SLRAEDTAVYYCARVGDGTTR | | TYYCQQYDTYWTFGQGTKVEI | |
| | FDSSGELDFWGQGTLITVSS | | K | |
| AB-007130 | QVQLVESGGGVVQPGRSLRLS | 75 | KIVMTQSPATLSVSPGERVTL | 536 |
| | CAASGFIFKNSGMHWVRQVPG | | SCRASQSISRKLAWYQQKPGL | |
| | QGLEWLALIWEDGSNEFYADS | | APRLLIYGASTRATGIPARFS | |
| | VEGRFTISRDNSKNTLYLQMN | | GSGSGTEFTLTISSLQSEDFA | |
| | SLRVEDTAVYYCARAGQFDNS | | LYYCQQYSNWYTFGQGTKLEI | |
| | GYLDYWGQGALVIVSS | | K | |
| AB-007168 | QVQLVESGGGLVQPGRSLRLS | 76 | QSVLTQPPSVSGAPGQRVTIS | 537 |
| | CEASGFIFSDCGMHWVRQTPG | | CTGGSSNIGAGYDVHWYQQLP | |
| | KGLEWVSVIWFDGSKQYYADS | | GTAPKLLIYGNTNRPSGVPDR | |
| | VKGRFTISRDNSKNMLYLQMN | | FSGSKSDTSASLAITGLQAED | |
| | SLSTEDSAVYYCARDIRGAYQ | | ETDYYCQSYDSSLRRYVFGSG | |
| | VADGGFDCWGQGTLVTVSS | | TKVTVL | |
| AB-007150 | DMQVVESGGGLVKPGGSLRLS | 77 | DIVMTQSPLSLPVTPGEPASI | 538 |
| | CAVSGFSVNNAWMSWVRQVPG | | SCRSSQSLLFSNGDNYLDWYL | |
| | KGLEWVGRIRSKTYGGTTDYA | | QKPGQSPQLLIYLGSYRASGV | |
| | APVKDRFTISRDDSKNTLYLQ | | PDRFSGSGSGTDFTLKISRVE | |
| | MNSLKSEDTGVYYCITDRDFY | | AEDVGVYFCMQSLQTPHTFGQ | |
| | RSGGYWGQGTLVTVSS | | GTKLEIK | |
| AB-007175 | QVQVVESGGGVVQPGRSLRLS | 78 | QSVLTQPPSVSGAPGQRVTIS | 539 |
| | CAASGFIFSNYGMHWVRQAPG | | CTGSSSNIGAGYDVHWYQQLP | |
| | KGLEWVAVIFYDGSKTYYADS | | GTAPKLLIYGNTNRPSGVPDR | |
| | AKGRFIISRDNSKNTLYLQMN | | FSGSKSGTSASLAITGLQAED | |
| | SLRAEDTAVYYCARAGYSDSS | | EADYYCQSYDSSLSSSWVFGG | |
| | GYGDYWGQGTLVTVSP | | GTKLTVL | |
| AB-000231 | QVHLVESGGGVVQPGRSLRLS | 79 | QSVLTQPPSVSGAPGQRVTIS | 540 |
| | CAASGFTFRNYGIHWVRQAPG | | CTGGSSNIGAGYDVHWYQQLP | |
| | KGLEWVAVIWYDGSREFYADS | | GTAPKLLISHNNNRPSGVPDR | |
| | VEGRFTVSRDNSKNTVFLQMN | | FSGSKSGTSASLVIAGLQAED | |
| | SLRVDDTAVYYCARAGYSDTS | | EADYYCQSYDRRLSSSWVFGG | |
| | GYGDHWGQGTLVSVSS | | GTRLTVLS | |
| AB-007109 | EVRLEESGGGLLQPGRSLRLS | 80 | HSVLTQPPSVSGAPGQRVTIS | 541 |
| | CTASGFRFGDYAMSWVRQAPG | | CTGSTSNIGAGYDVYWYQQLP | |
| | KGLEWVGFIRSKTFGGSTRNA | | GTAPKLLIYGNNNRPSGVPDR | |
| | ASLRGRFTLSRDDSKSVAYLQ | | FSGSTSGTSASLAITGLQAED | |
| | MNSLTTEDTAVYYCARVQLDS | | EADYYCQSYDTTLTGWEFGGG | |
| | GPGYYYYGMDVWGQGTTVIVS | | TKLTVL | |
| | S | | | |
| AB-007090 | QEHLVESGGGVVQPGKSLRLS | 81 | EIVLTQSPATLSLSPGEKATL | 542 |
| | CAASGFTFSNYGMHWVRQAPG | | SCRASQSVSTSLAWYQQKPGQ | |
| | RGLQWVAVIWYDGSQKFYAEF | | APRLLIYEASNRATGVPARES | |
| | ARGRFTVSRDNSKNTLYLQMN | | GSGSGTDFTLTISSLEPEDFA | |
| | SLRADDTAVYYCTRDYDYSSH | | VYYCQQRSSWFTFGQGTRLEI | |
| | NYFDPWGQGTLVTVSS | | K | |
| AB-007080 | QVHLVESGGGVVQPGRSLTVS | 82 | EIVLTQSPATLSLSPGERATL | 543 |
| | CAASGFTFRNYGMHWVRQVPG | | SCRASQSISSYLAWYQQKPGQ | |
| | KGLEWVGVIWHDGSQEHYGES | | APRLLIYDASTRSTGIPTRES | |
| | VEGRFSISRDNSKNTLYLQMN | | GSGSGTDFTLTISSLEPEDFA | |
| | SLGAEDTAVYHCARDRGYTGS | | IYFCQQRSSGFTFGPGTTVDV | |
| | WGYFDYWGQGTLVTVSS | | K | |
| AB-007102 | QVQLVESGGGVVQPGRSLRLS | 83 | DVVMTQSPLSLPVTLGQPASI | 544 |
| | CAASGFTFSYYGMHWVRQAPG | | SCRSSQSLVHSDGNTYLNWFQ | |
| | KGLEWVAIIWYDGSNKYYADS | | QRPGQSPRRLIYKVSNRDSGV | |
| | VKGRFTISRDNSKNTLYLQMN | | PDRFSGSGSGTDFTLKISRVE | |
| | SLRAEDTAVYYCARDDFDSGF | | AEDVGVYYCMQGTHWYTFGQG | |
| | YNHLAYWGQGTLVTVSS | | TKLEIK | |
| AB-007076 | QVQLAESGGGVVQPGRSLRLS | 84 | EIVLTQSPATLSLSPGERATL | 545 |
| | CAASGFIFRNYGMYWVRQAPG | | SCRASQSVSSYLAWYQQKPGQ | |
| | KGLEWLAVIWHDGSNEFYADS | | APRLLIYDASKRATDIPARFS | |
| | VEGRFTISRDNSKNTLFLHMS | | GSGSGTDFTLTISSLEPEDFA | |
| | SLRVEDTAVYYCVRDRGYTDS | | VYYCQQRSVGFTFGPGTKVDI | |
| | WGYFGFWGQGTLVTVSS | | K | |
| AB-007106 | QVQLVESGGGVVQPGRSLRLS | 85 | DIQMTQSPSTLSASVGDRVTI | 546 |
| | CAASGFTFGSYGMHWVRQAPG | | TCRASQSISRWLAWYQQKPGK | |
| | KGLEWVAIIWYDGSNKYYADS | | APKLLIYKASSLQSGVPSRFS | |
| | VKGRFTISRDNSKNTLYLQMN | | GSGSGTEFTLTISSLQPDDFA | |
| | SLRAEDTAVYYCARVYQPTDY | | TYYCQQYNSYWTFGQGTKVEI | |
| | YDSSRGDAFDIWGQGTMVTVS | | K | |
| | S | | | |
| AB-007107 | QVQLVESGGGVVQPGRSLRLS | 86 | DIQMTQSPSTLSASVGDRVTI | 547 |
| | CAASGFTFRSYGMHWVRQAPG | | TCRASQSVSRWLAWYQQKPGK | |
| | KGLEWVAVIWYDGSNKYYPDS | | APKLLIYDASSLESGVPSRFS | |
| | VKGRFTISRDNSQNTLFLQMN | | GSGSGTEFTLTISSLQPDDFA | |
| | SLRAEDTAVYYCARVGEYSSS | | TYYCQQYNRYWTFGQGTKVEI | |
| | PDWGAAFDIWGQGTMVIVSS | | K | |
| AB-007111 | EVQLVESGGGLGQPGRSLRLS | 87 | QSVLTQPPSVSGAPGQRVTIP | 548 |
| | CTASGFTFGDYAMSWVRQAPG | | CTGSSSNIGAGYDVYWYQQLP | |
| | KGLEWLGFIRSKTYGGSTKYA | | GTAPKLLIYGDTNRPSGVPDR | |
| | APVKARVIITRDDSKSIAYLQ | | FSGSKSGTSASLAITGLQAED | |
| | MNSLKTEDTAVYYCARVQLDS | | EADYYCQSYDSTVSGWEFGGG | |
| | GPGYYYYGMDVWGQGTTVTVS | | TKLTVL | |
| | S | | | |
| AB-007159 | EVQLVESGGGLVQPGRSLRLS | 88 | DIVMTQTPLSLSVTPGQPASI | 549 |
| | CTTSGFTFGDYAMSWFRQAPG | | SCKSSQSLLDNDGKTYLYWYL | |
| | KGPEWVGFIRSKGYGGTAEYA | | QKPGQSPQLLIYEVSNRFSGV | |
| | ASVKGRFVISRDDAKSIAYLQ | | PDRFSGSGSGTDFTLKISRVE | |
| | MNSLKTEDTAIYYCARVGILA | | AEDVGVYYCMQRIDLPWTFGQ | |
| | ASAVDWGQGTLVAVSP | | GTKVEIK | |
| AB-007170 | QVQLVESGGGVVQPGRSLRLS | 89 | QSVLTQPPSVSGAPGQTVTIS | 550 |
| | CATSGFTFSNYGMHWVRQAPG | | CTGSSSNIGANYDVHWYQQRP | |
| | KGLEWVAVTWHDGSKKFYADS | | GTAPKLLIYGNMNRPSGVPDR | |
| | VDGRFTISRDNSKNTVSLQMN | | FSASKSGTSASLAITGLQADD | |
| | SLRVEDTAVYYCARAAYYDTS | | EADYYCQSYDRRLMASWVFGG | |
| | GYGDYWGHGTLVTVSS | | GTKLTVL | |
| AB-007165 | QVQLVESGGGVVQPGRSLRLS | 90 | QSVLTQPPSVSGAPGQRVTIS | 551 |
| | CAATGFTFSNFGMHWVRQAPG | | CTGSSFNIGAGYDVHWYQQIP | |
| | KGLEWVAVIWHDGSQKYYGEF | | GTAPKVLIFGYTNRPSGVPDR | |
| | VKGRFTISRDNSQNTLHLQLN | | FSGSKSGTSASLAITGLQAED | |
| | SLRVDDTATYFCARDIRGAYQ | | EADYYCQSYDSSLSGYVFGTG | |
| | ATDGGFDSWGQGTQVTVSS | | TKVTVL | |
| AB-007115 | EVQLVESGGGWVQPGRSLRLP | 91 | DIVLTQTPLSLSVTPGQPASM | 552 |
| | CTTSGFSFEDYAMSWFRQAPG | | SCKSSQSLLYSDVKTYLHWYL | |
| | RGLEWVGFIRSKANGGATESA | | QKPGQPPQLLMYEVSNRFSGV | |
| | ASVKGRFTISRDDPKSIAYLQ | | PDRFSGSGSGTDFTLKISRVE | |
| | MNSLKTEDTAVYYCARVRTND | | AEDVGVYYCMQTIDLPWTFGQ | |
| | FRDMDVWGKGTTVIVSS | | GTKVEIK | |
| AB-007138 | ELQLVESGGGLVQPGGSLRLS | 92 | VITMTQTPLSLSVTPGQPASI | 553 |
| | CAASGFTFSNYWMSWVRQAPG | | SCKSSQSLLHSDGKTYLYWYL | |
| | KGLEWVANINQDETIKYYLDS | | QKPGQSPQLLMYEVSKRVSGA | |
| | VKGRFTVSRDNAKNLLYLQMN | | PDRFSGSGSGTDFTLKISRVE | |
| | DLRAEDTAVYYCARYLNYWGQ | | SEDVGVYYCMQSIEIPLTFGG | |
| | GILVTVSS | | GTRVEIK | |
| AB-007160 | EVQLVESGGGLVQPGRSLRVS | 93 | DIVMTQTPLSLSVTPGQPASI | 554 |
| | CTTSGFTFGDYAMSWFRQAPG | | SCKSSQSLLDNDGKTYLYWYL | |
| | KGLEWVGFIRSKGYGGTPEYA | | QKPGQSPQLVIYEVSNRFSGV | |
| | ASVKGRFTISRDDSKRTAYLQ | | PDRFSGSGSGTDFTLKIRRVE | |
| | MNSLKTEDTAVYYCTRVGIEV | | AEDVGVYYCMQRIDLPWTFGQ | |
| | STAVYWGPGTLVTVSS | | GTKVEIK | |
| AB-007112 | EVQLVESGGGLVQPGRSLRLS | 94 | QSVLTQPPSVSGAPGQRVTIS | 555 |
| | CTASGFSFGDHALSWVRQAPG | | CTGSSSNIGAGYDVYWYQQLP | |
| | KGLEWVGFIRSKTYGGSTKYA | | GTAPKLLIYGNDNRPSGVPDR | |
| | ASVKGRVTMSRDDAKRIAYLQ | | FSGSKSGTSASLAITGLQAGD | |
| | INSLKTDDTAVYYCARVQLDP | | EADYYCQSYDTIVSGWEFGGG | |
| | GPGYYYYGMDVWGQGTTVTVS | | TKLTVL | |
| | S | | | |
| AB-007142 | EVQVVESGGGLVKPGGSLRLS | 95 | DIVMTQSPLSLPVTPGEPASI | 556 |
| | CAASGFTFSKAWMSWVRQAPG | | SCRSSQSLLFSNGDNYLDWYL | |
| | KGPEWVGRIESRTDGGTIEYA | | QKPGQSPQLLIYLGSNRAPGV | |
| | AAVKGRFTISRDDSKDTLYLQ | | PDRFSGSGSGTDFTLKISRVE | |
| | MNSLKTEDTAVYYCITDRDFY | | AEDVGVYYCMQALQTPHTFGQ | |
| | RSGGHWGQGTLVTVSS | | GTKLEIR | |
| AB-007126 | EGQLVESGGGLVQPGGSLKLS | 96 | DVVMTQSPLSLPVTLGQPASI | 557 |
| | CAASGFTFSEYEMNWVRQAPG | | SCRSSQSLLYSDGNTYLFWFQ | |
| | KGLEWVSYISRSGSPRHYADS | | QRPGQSPRRLIYKVSNRDSGV | |
| | VKGRFTVSRDNTKNSLFLQMN | | PDRFSGSGSGTDFTLKISRVE | |
| | SLRVEDTAVYYCARDHSTSAT | | AEDVGVYFCLQGTHWPYTFGP | |
| | EYGLDVWGQGTTVIVSS | | GTKLEIK | |
| AB-007113 | EVQLVESGGGLVQPGRSLRLS | 97 | QSVLTQPPSVSGAPGQRVTIS | 558 |
| | CTASGFIFPEYAMSWVRQAPG | | CTGSSSNIGAGYDVYWYQQFP | |
| | KGLEWLGFIRKKAYGGTTNYA | | GTAPKLLIYGDSHRPSGVPDR | |
| | ASVKGRITLSRDDSKSIAYLQ | | FSGSKSGASASLAITGLQAAD | |
| | MNSLKIEDTAVYYCTRVQLDS | | EADYYCQSYDTSLSGWEFGGG | |
| | GPGYYYYGMDVWGQGTTVTVS | | TKVTVL | |
| | S | | | |
| AB-007108 | EVQLVESGGGLVQPGRSLRLS | 98 | QSGVTQPPSVSGAPGQRVTIS | 559 |
| | CAASGFNFGDYAMSWVRQAPG | | CTGSSSNIGAGYDVYWYQQLP | |
| | KGLEWVGFIRKKAYGGTTRYA | | GTAPKLLIYGNTNRPSGVPDR | |
| | ASEKGRFTISRDDSKSIAYLQ | | FSGSKSGTSASLAITGLQAED | |
| | MNSLKTEDTAVYYCARVQLDS | | EADYYCQSYDTSLSGWVFGGG | |
| | GPGYYYYGMDIWGQGTTVTVS | | TKLTVL | |
| | S | | | |
| AB-007118 | EVQVVESGGGVVQPGRSLRLS | 99 | DIVMTQTPLSLSVTPGQPASI | 560 |
| | CTASGFTFGDYAMSWFRQAPG | | SCKSTESLLHRDGKTYLYWYL | |
| | KGLEWVGFIRSEANGGAPEYA | | QKPGQPPQLLIYEVSNRFSGV | |
| | ASVKGRFTISRDDFKSIAYLQ | | PDRFSGSGSVTDFTLKISRVE | |
| | MNSLKTEDTAAYYCTRVRTND | | AEDIGVYYCMQTIDLPWTFGQ | |
| | FRDMDVWGKGTTVAVSS | | GTKVEIK | |
| AB-007158 | EVQLVESGGGLVQPGRSLRLS | 100 | DILMTQTPLSLSVTPGQPASI | 561 |
| | CTTSGFTFGDYAMSWFRQAPG | | SCKSSQSLLDNDGKTYLYWYL | |
| | KGLEWVGFIRSTGFGGSAEYA | | QKPGQSPQLLIYEVSNRFSGV | |
| | AFVKGRFTISRDDSKSIAYLQ | | PDRFSGSGSGTDFTLRIRRVE | |
| | MNILKPEDTAVYLCSRVGVGI | | AEDVGVYYCFQRIDLPWTFGQ | |
| | STAVNWGQGTLVTVSS | | GTKVEIK | |
| AB-007169 | QVQLVESGGGVVQPGKSLTLS | 101 | QSVLTQPPSVSGAPGQRVTVS | 562 |
| | CAASGFSFRNYGMHWVRQAPG | | CTGSGSNIGANYEVHWYQQLP | |
| | KGLEWVAVIWEDGSKEYYADS | | GTAPKLLIYGDNNRPSGLPDR | |
| | VYGRFAISRDNSKNTLYLQMN | | FSASKSGTSASLAITGLQAED | |
| | TLRVEDTAIYYCATDIRGAYK | | EADYYCQSYDSSLSGYVFGSG | |
| | VTDGGFGHWGQGTLVTVSP | | TKVTVL | |
| AB-007139 | EMQLVESGGGLVQPGGSLRLS | 102 | EIVMTQTPLSLSVTPGQPASI | 563 |
| | CAASGFTFRNYWMSWVRQAPG | | SCKSSQSLLHSDGKTYLYWYL | |
| | KGLEWVANINQDESIKYYLDS | | QKPGQSPQLLIYEVSKRFSGV | |
| | VKGRFTISRDNDKNLLFLQMS | | PDRFSGSGSGTDFTLQISRVE | |
| | GLRAEDTSIYYCARYLNFWGQ | | AEDVGVYYCLQTIEIPLTFGG | |
| | GALVTVSS | | GTKVEIK | |
| AB-000245 | EVQLVESGGGLVKPGGSLRLS | 103 | SFVLTQPPSVSVAPGQTASIT | 564 |
| | CAASGFTFSSYTMIWVRQAPG | | CGGNNIGSKSVHWYQQKPGQA | |
| | KGLEWVSSISSSSSKIYHPNS | | PVLVVYDDSVRPSGIPERFSG | |
| | LKGRFTVSRDNAKNSVFLHMN | | SNSGNTATLTITRVEAGDEAD | |
| | SLRAEDTAIYYCARTELLAAA | | YSCQVWDTSSDRLYVFGTGTR | |
| | ATGGIEYWGQGTLVTVSS | | VTVL | |
| AB-000389 | EVQLVESGGGLVQPGGSLRLS | 104 | DIQMTQSPSTLSASVGDRVTI | 565 |
| | CAGSGFTFTYYGMNWIRQAPG | | TCRASQSISTWLAWYQQRPGK | |
| | KGLEWVSYISVGSDIIHYADS | | APKLLIYKASSLETGVPSRFS | |
| | VKGRFSISRDNAKKSLYLQMN | | GSGSGTEFTLTISSLQPDDFA | |
| | SLRVEDSAVYFCVRGVSSGHY | | TYYCHQYSSYSPRSFGQGTKL | |
| | GTEDLLDYWGQGTLVTVSS | | EIK | |
| AB-000310 | QVQLVESGGGVVQPGRSLRLS | 105 | DIQMTQSPSSLSASVGNRVTI | 566 |
| | CEASGFTFSDYAMHWVRQAPG | | TCRASQIVGSSLHWYQQKPGK | |
| | KGLEWVTFMSYDGRNEYYADS | | APKLLIHHASSLQSGVPSRFS | |
| | VKGRFTISRDNSKNTLYLQMT | | GSGSGTDFTLTIGSLQPEDFA | |
| | SLRRDDTAVYYCARDRVGSLV | | TYYCQQSYSTPYTFGQGTNLE | |
| | YWGQGTLVTVSS | | IK | |
| AB-000335 | EVLLVESGGGLVKPGGSLRLS | 106 | PYLLTQPPSVSVAPGTTARIT | 567 |
| | CAASGFTFSSSTMNWVRQAPG | | CGENNIGSQSVHWYQQKPGQA | |
| | KGLEWVASISSRSSYIYYADS | | PVLVVYDDRVRPSGIPERFSG | |
| | LKGRFTVSRDNANNSVYLQMN | | SNSGNTATLTISRVEAGDEAD | |
| | SLRAEDTAIYFCARGGGMGLV | | YYCHVWYSSAHHQVFGGGTKL | |
| | TTGYFEYWGQGTLVTVSS | | TVL | |
| AB-000367 | EVQLVESGGGLVKPGGSLRLS | 107 | SYELTQPPSLSVSPGQTATIT | 568 |
| | CAASGFTFNTYTMNWVRQAPG | | CSGDGLGDNYASWYQQKPGQS | |
| | KGLEWVSSISSSGSKKYYGDS | | PVLVIYQDNKRPSGIPERFSG | |
| | VKGRFSISRDNARNSLYLQMN | | SNSGNTATLTISGTQAMDEAD | |
| | SLRDEDTAVYYCVRDGNVMIR | | YYCQTWDSSTLWVFGGGTKLT | |
| | GTGDWFDPWGQGTLVTVTS | | VL | |
| AB-000369 | QVHLQQSGPGLVQPSETLSLT | 108 | QSVLTQPPSVSGAPGQRVTIS | 569 |
| | CSVSGGSISGFYWSWIRQPPG | | CTGSSSNIGADYDVHWYQHLP | |
| | KGLEWIGFIYRSASTDYNPSL | | GTAPKLLIYGNTNRPSGVPDR | |
| | KSRVTISLDTSKNQFSLKLNS | | FSGSKSGTSASLAITGLQAED | |
| | VTAADTAVYYCARDTGSYMKG | | EADYYCQSYDSSLYVVFGGGT | |
| | WGDYGMDVWGQGTTVTVSS | | KLTVL | |
| AB-000386 | EVRLVESGGGLVKPGGSLRLS | 109 | QSVLTQPPSVSGAPGQRVTIS | 570 |
| | CGASGFTFNTYTMNWVRQAPG | | CTWSSSNIGAALDVHWYQQVP | |
| | KGLEWVSSISSGSGKIYYADS | | GTAPKLLIYSNTNRPSGVPDR | |
| | VKGRFTISRDNAKNSLYLQMN | | FSGSKSATSASLAITGLQAED | |
| | GLRVEDTAIYYCAREERVGNW | | EADYYCQSYDSSLSVLFGGGT | |
| | YSGGMDVWGQGTPVTVSS | | KLTVL | |
| AB-001390 | QVQLVESGGGVVQPGRSLRLS | 110 | DIVMTQSPLSLPVTPGEPASI | 571 |
| | CAASGFTFSSYGMHWVRQAPG | | SCRSSQSLLHSNGYNYLDWYL | |
| | KGLEWVAVIWYDGSNKYYADS | | QKPGQSPQLLIYLGSNRASGV | |
| | VKGRFTISRDNSKNTLYLQMN | | PDRFSGSGSGTDFTLKISRVE | |
| | SLRAEDTAVYYCASGVTTGWD | | AEDVGVYYCMQALQSYTFGQG | |
| | EIHDAFDIWGQGTMVTVSS | | TKLEIK | |
| AB-001403 | QVQLVESGGGVVQPGRSLRLS | 111 | QSVLTQPPSVSGAPGQRVTIS | 572 |
| | CAASGFTFSSYGMHWVRQAPG | | CTGSSSNIGAGYDVHWYQQLP | |
| | KGLEWVAVIWYDGSNKYYADS | | GTAPKLLIYGNSNRPSGVPDR | |
| | VKGRFTISRDNSKNTLYLQMN | | FSGSKSGTSASLAITGLQAED | |
| | SLRAEDTAMYYCAKDYYYDSS | | EADYYCQSYDSSLSGLVFGGG | |
| | GQTRLFDYWGQGTLVTVSS | | TKLAVL | |
| AB-001405 | QEQLVESGGGVVQPGRSLRLS | 112 | DIVMTQTPLSLSVTPGQPASI | 573 |
| | CAASGFTFSSYGMHWVRQAPG | | SCKSSQSLLHSDGKTYLYWYL | |
| | KGLEWVALIWYDGSNKYYADS | | QKPGQAPQLLIYEASKRFAGV | |
| | VKGRFTVSRDNSKETLYLQMN | | PDRFRGSGSGTDFTLKISRVE | |
| | SLRDEDTAVYYCVREYSSSWT | | AEDVGVYYCMQSIQLWTFGQG | |
| | GWFDPWGQGTLVTVSS | | TKVEIK | |
| AB-001409 | QVQLVESGGGVVQPGRSLRLS | 113 | DIQMTQSPSTLSASVGDRVTI | 574 |
| | CAASGFTFSIYGMHWVRQAPG | | TCRASQSISRWLAWYQQKPGK | |
| | KGLEWVAVIWHDGSIKYYAES | | APKLLIYKASNLESGVPSRFS | |
| | VKGRFTISRDNSKNTLYLQMN | | GSGSGTEFTLTISSLQPDDFA | |
| | SLRVEDTAVYYCAKVGEGEDY | | TYYCQQYKTLWTFGQGTKVEI | |
| | SDSSGYFDYWGQGTLVTVSS | | K | |
| AB-001410 | QVQLVESGGGVVQPGRSLRLS | 114 | DIQMTQSPSTLSASVGDRVTI | 575 |
| | CAASGFPFSTYGMHWVRQTPG | | TCRASQSISTWLAWYQQKPGK | |
| | KGLEWVAIIWHDGSKKYYADS | | APKLLIYKASTLESGVPSRFS | |
| | VEGRFTISRDNSKKTLYLQMN | | GSGSGTEFTLTISSLQPDDFA | |
| | SLRTEDTAVYYCARPGERRLQ | | TYYCQQYNFYWTFGQGTKVEV | |
| | KSGDFDKWGQGTLVTVSS | | K | |
| AB-001411 | QVQLVESGGGVVQPGRSLRLS | 115 | DIVMTQSPLSLPVTPGEPASI | 576 |
| | CAASGFTFNSYGMHWVRQAPG | | SCRSSQSLLHSNGYNYLDWYL | |
| | KGLEWVAVIWYDGSNKYYADS | | QKPGQSPQLLIYLGSNRASGV | |
| | VKGRFTISRDNSKNTLYLQMN | | PDRFSGSGSGTDFTLKISRVE | |
| | SLRAEDTAVYYCAKDGRDSSD | | AEDVGVYYCMQALQTWTFGQG | |
| | NGGIFDYWGQGTLVTVSS | | TKVEIK | |
| AB-001413 | QVQLVESGGGVVQPGRSLRLS | 116 | DIQMTQSPSTLSASVGDRVTI | 577 |
| | CAASGFTFSSYGMHWVRQAPG | | TCRASQSISSWLAWYQQKPGK | |
| | KGLEWVAVIWYDGSNKFYADS | | APKLLIYKASSLESGVPSRFS | |
| | VKGRFTISRDNSKNTLYLQMN | | GSGSGTEFTLTISSLQPDDFA | |
| | SLRVDDTAVYYCAKALGGEEG | | TYYCQQYNSYFTFGQGTKLEI | |
| | DSCCPFDIWGQGTMVTVSS | | K | |
| AB-001429 | QVQLVESGGGVVQPGRSLRLS | 117 | QSVLTQPPSVSGAPGQRVTIS | 578 |
| | CAASGFTFSNYGMHWVRQAPG | | CTGSSSNIGAGYDVHWYQQLP | |
| | KGLEWVAVIWYDGSKKYYADS | | GTAPKLLIYSNSNRPSGVPDR | |
| | LKGRFTISRDNSKNTLYLQMT | | FSGSKSGTSASLAITGLQAED | |
| | SLRAEDTAVYYCARDGRGDYG | | EADYYCQSYDSSLSGLYVFGT | |
| | GNSAHFDYWGQGTLVTVSS | | GTKVTVL | |
| AB-001439 | QEQLVESGGGVVQPGRSLRLS | 118 | QSVLTQPPSVSGAPGQRVTIS | 579 |
| | CAASGFTFSSYGMHWVRQTPG | | CTGSSSNIGAGYDVHWYQQLP | |
| | RGLEWVAVIWYDGSKKYYGDS | | GTAPKLLIYGNTNRPSGVPDR | |
| | VRGRFTISRDNSKNTLYLQMN | | FSGSKSGTSASLAITGLQAED | |
| | SLRAEDTAVYYCARDLRGGYY | | EADYYCQSYDSSLSDYVFGTG | |
| | LRDGGFDYWGQGTLVTVSS | | TRVTVL | |
| AB-001449 | EVQLVESGGVLVKPGGSLRLS | 119 | DIVLTQSPVSLPVTPGEPASI | 580 |
| | CAASGFTLTDAWMSWVRQAPG | | ACRSSQSLLYSNGDYYLDWYL | |
| | KGLEWVGRIKSKTHGGTTDYA | | QKPGQSPQLLIYLGSNRASGV | |
| | APVKARFTISRDESKSTLYLQ | | PDRFSGSGSGTDFTLKISGVE | |
| | MNSLKTEDTAVYYCTTAEARR | | AEDVGIYYCMQSLQTPLTFGG | |
| | DLTSWGQGTLVTVSS | | GTKVEIK | |
| AB-001467 | QVQLVESGGGVVQPGRSLRLS | 120 | EIVLTQSPATLSLSPGERATL | 581 |
| | CTASGFTFSNYGMHWVRQAPG | | SCRASQSVSSYLAWYQQKPGQ | |
| | KGLEWVAVIWYDGSNKYYADS | | APRLLIYDASNRATGIPARFS | |
| | VKGRFTISRDNSKNTLYLQMN | | GSGSGTDFTLTISSLEPEDFA | |
| | SLRAEDTAVYYCARDGPATVT | | IYYCQQRSNWWTFGQGTKVEI | |
| | TWNYFDYWGQGTLVTVSS | | K | |
| AB-001471 | QVQLVESGGGVVQPGRSLRLS | 121 | EIVMTQSPVTLSVSPGERATL | 582 |
| | CAASGFTFSNYGMHWVRQAPG | | SCRASQSVSSDLAWYQQKPGQ | |
| | KGLEWVSLISYDGGRTFYADS | | APRLLIYGASTRATGIPARFS | |
| | VKGRFTISRDNSRNTLYLQMN | | GSGSGTEFTLTISSLQSEDFA | |
| | SLRTEDTAMYYCARVGYGSYY | | VYYCHQYNNWYTFGQGTKLEI | |
| | TATTNPVDHWGQGTLVTVSS | | K | |
| AB-001474 | QVQLVQSGAEVKKPGASVKVS | 122 | DIVMTQSPLSLPVTPGEPASI | 583 |
| | CKASGYTGYYIHWVRQAPGQG | | SCRSSLSLLHSNGYNYLDWYL | |
| | LEWMGWINPNDGGTIYALKFQ | | QKPGQSPQLLIYLGSNRASGV | |
| | GRVTMTRDTSISTTYMELSRL | | PDRFSGSGSGTDFTLKISRVE | |
| | SSDDTAIYYCATLTVAAAATD | | AEDVGVYYCMQALETPITFGQ | |
| | YWGQGTLVTVSS | | GTRLEIK | |
| AB-001476 | QVQLVQSGAEVKKPGASVKVS | 123 | QPVLTQPPSASASLGASVTLT | 584 |
| | CKASGYTFTSYDINWVRQATG | | CTLSSGYSNYKVDWYQQRPGK | |
| | QGLEWMGWMNPNSGNTVYAQK | | GPRFVMRVGTGGIVGSKGDGI | |
| | FQGRVTMTRNTSISTAYMELS | | PDRFSVLGSGLNRYLTIKNIQ | |
| | SLRSEDTAVYYCARGGYCSSS | | EEDESDYHCGADHGSGINFVW | |
| | SCYEVEFDPWGQGTLVTVSS | | VFGGGTKLTVL | |
| AB-001481 | QVQLVESGGGVVQPGRSLRLS | 124 | DVVMTQSPLSLPVTLGQPASI | 585 |
| | CAASGFTFSSYGMHWVRQAPG | | SCRSSQSLVYSDGNTYLNWFQ | |
| | KGLEWVAVIWYDGSNKYYADS | | QRPGQSPRRLIYKVSNRDSGV | |
| | VKGRFTISRDNSKNTLYLQMN | | PDRFSGSGSGTDFTLKISRVE | |
| | SLRAEDTAVYYCAKVGEYGDY | | AEDVGVYYCMQGTHRPLFTFG | |
| | GDGMDVWGQGTTVTVSS | | PGTKVDIK | |
| AB-001486 | QVQLVESGGGVVQPGRSLRLS | 125 | DIQMTQSPSTLSASVGDRVTI | 586 |
| | CASSGFTFSGYGMHWVRQTPD | | TCRASQNIGTWLAWYQQRPGK | |
| | KGLEWVAIIWYDGSKKYYADS | | APKLLIYKASSLQTGVPSRFS | |
| | VKGRFTISRDNSRNTLYLQMN | | GSGSGTEFTLTISSLQPDDFA | |
| | SLRAEDTAVYYCARVLNDHVD | | SYYCQQYNFYWTFGQGTKVEI | |
| | YGSKSGAFDIWGQGTMVTVSS | | K | |
| AB-001493 | QVQLVESGGGVVQPGRSLRLS | 126 | QSVLTQPPSVSGAPGQRVTIS | 587 |
| | CAASGFTFSSYGMHWVRQAPG | | CTGSSSNIGAGYDVHWYQQLP | |
| | KGLEWVAVIWYDGSNKYYADS | | GTAPKLLIYGNSNRPSGVPDR | |
| | VKGRFTISRDNSKNTLYLQMN | | FSGSKSGTSASLAITGLQAED | |
| | SLRAEDTAVYYCARDRSHLDR | | EADYYCQSYDSSLSGLLFGGG | |
| | TRAFDPWGQGTLVTVSS | | TKLTVL | |
| AB-001503 | QVQLVESGGGVVQPGRSLRLS | 127 | DVVMTQSPLSLPVTLGQPASI | 588 |
| | CAASGFTFSSYGMHWVRQAPG | | SCRSSQSLVHSDGNTYLNWFQ | |
| | KGLEWVAIIWYDGSNKYYADS | | QRPGQSPRRLIYKVSNRDSGV | |
| | VKGRFTISRDNSKNTLYLQMN | | PDRFSGSGSGTDFTLKISRVE | |
| | SLRAEDTAVYYCAKDGVDYYD | | AEDVGVYYCMQGTHWWTFGQG | |
| | SSGYSQLDNWGQGTLVTVSS | | TKVEIK | |
| AB-001528 | QEQLVESGGGVVQPGRSLRVS | 128 | EIVMRQSPATLSVSPGERATL | 589 |
| | CAASGFTFSNHGMHWVRQAPG | | SCRASQSVSSNLAWYQLKPGQ | |
| | KGPEWVAVIWHDGSKKFYADS | | APRLLIFGASTRATGIPARES | |
| | IKGRFTISRDNSKNTLYLQMN | | GSGSGTEFTLTISNLQSEDFA | |
| | SLTAEDTAVYYCARVMASSRS | | VYYCQQYNDWWTFGQGTKVEI | |
| | SELPFDIWGLGTMVTVSS | | K | |
| AB-001531 | QVQLVESGGGVVQPGRSLRLS | 129 | DIQMTQSPSTLSASVGDRVTI | 590 |
| | CAASGFTFSSYGMHWVRQTPG | | TCRASHSIYTWLAWYQQKPGK | |
| | KGLEWVAVVWYDGSNKYYAES | | APKLLIYKASTLESGVPSRFS | |
| | VKGRFTISRDNSKNTLYLQMN | | GSGSGTEFTLTISSLQPDDFA | |
| | SLRAEDTAVYYCARVLDYGDY | | TYYCQQYNRYWTFGQGTKVEI | |
| | GQSEDRFGAFDIWGQGTLVTV | | K | |
| | SS | | | |
| AB-001532 | QVQLVESGGGVVQPGRSLRLS | 130 | QSALTQPASVSGSPGQSITIS | 591 |
| | CAASGFTFRNYGMHWVRQAPG | | CTGTSSDVQSYNYVSWYQQHP | |
| | KGLEWVAVIWYDGSREFYADS | | GKAPKLIIFEVSNRPSGVSNR | |
| | VEGRFTTSRDNSKNTLYLQMN | | FSGSKSGNTASLNISGLHGED | |
| | SLRAEDTAVYYCARDRGGSHD | | EADYYCSSKRVFGGGTKLTVL | |
| | REFDYWGQGTLVTVSS | | S | |
| AB-001535 | EVQLVESGGGLVQPGRSLRLS | 131 | EIVLTQSPATLSLSPGERATL | 592 |
| | CTASGFTFGDYAMSWVRQAPG | | SCRASHSVSSYLAWYQQKPGQ | |
| | KGLEWVGFIRSKAHGGTTEYA | | APRLLIYDASNRATGIPARFS | |
| | ASVKGRFTISRDDSKSIAYLQ | | GSGSETDFTLTISSLEPEDFA | |
| | MNSLKTEDTAVYYCSRVSLVP | | VYYCQQRNSWWTFGQGTKVEI | |
| | AAPLDYWGQGTLVTVSS | | K | |
| AB-001536 | EVQLVESGGGLVQPGRSLRLS | 132 | EIVMTQTPLSLSVTPGQPASI | 593 |
| | CTASGFTFADYAMSWVRQAPG | | TCKSSQSLLNTDGKTYLYWYL | |
| | KGLEWVGFIRSKAFGGSTEYA | | QKPGQPPQLLIYEVSNRFSGV | |
| | ASVKGRFTISRDDSKSIAYLQ | | PDRFSGSGSGTDFTLKISRVE | |
| | MNGLKTEDTAVYSCTRIGIAA | | AEDVGVYYCMQSIQLPWTFGQ | |
| | RACDYWGQGTLVTVSS | | GTKVEIK | |
| AB-001537 | QVQLVESGGGVVQPGRSLRLS | 133 | DIQMTQSPSTLSASVGDRVTI | 594 |
| | CAASGFTFSSYGMHWVRQAPG | | TCRASQSISSWLAWYQQKPGN | |
| | KGLEWVAVIWYDGSNKYYADS | | APKLLIYKASSLESGVPSRFS | |
| | VKGRFTISRDNSKNTLYLQMN | | GSGSGTEFTLTISSLQPDDFA | |
| | SLRAEDTAVYYCARVADEVLE | | TYYCQQYNSYWTFGQGTKVEI | |
| | WSGAFDYWGQGTLVTVSS | | K | |
| AB-001538 | EVQLLESGGGLVQPGGSLRLS | 134 | DIVMTQSPDSLAVSLGERATI | 595 |
| | CAASGFTFSSYAMSWVRQAPG | | NCKSSQSVLYSSNNKNYLAWY | |
| | KGLEWVSVIYNDGTSTYYADS | | QQKPGQPPKLLIYWASTRESG | |
| | VKGRFTISRDNSKNTLYLQMN | | VPDRFSGSGSGTDFTLTISSL | |
| | SLRAEDTAVYYCAKEDYGNNS | | QAEDVAVYYCQQYYSAPWTFG | |
| | NYWGQGTLVTVSS | | QGTKVEIK | |
| AB-001540 | QVQLVESGGGVVQPGRSLRLS | 135 | DIVMTQSPDSLAVSLGEGATI | 596 |
| | CAASGFTFSSYGMYWVRQAPG | | NCKSSQSVLYTSNNKNYLAWY | |
| | KGLEWVAVIWYDGSNKHYADS | | QQKPGQPPKLLIYWASTRESG | |
| | VKGRFTISRDNSKNTVYLQMN | | VPDRFSGSGSGTDFTLTISSL | |
| | SLRAEDTAVYYCAKGKGEGWF | | QAEDVAVYYCQQYYSTPLTFG | |
| | DPWGQGTLVTVSS | | GGTKVEIK | |
| AB-001541 | QVQLVESGGGVVQPGRSLRLS | 136 | DIQMTQSPSSLSASVGDRVTI | 597 |
| | CAASGFTFSSYGMHWVRQAPG | | TCRASQSISSYLNWYQQKPGK | |
| | KGLEWVAVIWYDGSYKYYADS | | APKLLIYAASSLQSGVPSRFS | |
| | VKGRFTVSRDNSKNTLYLQMN | | GSGSGTDFTLTISSLQPEDFA | |
| | SLRAEDTAVYYCARAYYDSSG | | TYYCQQSYSSFTFGPGTKVDI | |
| | PTAFDYWGQGTLVTVSS | | K | |
| AB-001547 | EVQLVQSGAEVKKPGESLKIS | 137 | DVVMTQSPLSLPVTLGQPASI | 598 |
| | CKGSGYSFTSCWIGWVRQMPG | | SCRSSQSLVHNDGNTYLNWFQ | |
| | KGLEWMGIIYPGDSDTRYSPS | | QRPGQSPRRLIHKVSNRDSGV | |
| | FQGQVTISADKSISTAYLQWS | | PDRFSGSGSGTDFTLKISRVE | |
| | SLRASDTAMYYCARHYCDSSG | | AEDVGVYYCMQGTHWPYTFGQ | |
| | YYYKDYWGQGTLVTVSS | | GTKLEIK | |
| AB-001555 | QVQLVESGGGVVQPGRSLRLS | 138 | DIQMTQSPSTLSASVGDRVTI | 599 |
| | CAASGFTFSSYGMHWVRQAPG | | TCRASQSISSWLAWYQQKPGK | |
| | KGLEWVAVIWYDGSNKYYADS | | APKLLIYKASSLESGVPSRFS | |
| | VKGRFTISRDNSKNTLYLQMN | | GSGSGTEFTLTISSLQPDDFA | |
| | SLRAEDTAVYYCAKVRYSSSW | | TYYCQQYNSYWTFGQGTKVEI | |
| | YGDAFDIWGQGTMVTVSS | | K | |
| AB-001561 | QVQLVESGGGVVQPGRSLRLS | 139 | DIVMTQTPLSSPVTLGQPASF | 600 |
| | CAASGFTFSSYGMHWVRQAPG | | SCRSSQSLVHSDGNTYLSWLQ | |
| | KGLEWVAIIWYDGSYKYYADS | | QRPGQPPRLLIYKISNRFSGV | |
| | VKGRFTISRDNSKNTLYLQMD | | PDRFSGSGAGTDFTLKISRVE | |
| | SLRAEDTAVYYCARALGNDYG | | AEDVGVYYCMQATQWFTFGQG | |
| | DYGGFDYWGQGTLVTVSST | | TKLEIK | |
| AB-001566 | QVQLVESGGGVVQPGRSLRLS | 140 | KIVMTQSPATLSVSPGEGATL | 601 |
| | CAASGFTFSNYGMHWVRQAPG | | SCRASQSVSSNLAWYQQKPGQ | |
| | KGLEWVALIWYDGSNEFYADS | | APRLLIYGASTRATGIPARFS | |
| | VEGRFTISRDNSQNTLYLQMN | | GSGSGTEFTLTISSLQSEDFA | |
| | SLRAEDTAVYYCARSGAYHHG | | VYYCQQYNHWFTFGPGTKVDI | |
| | SSGYVDYWGRGTLVTVSS | | K | |
| AB-001568 | QVRLVQSGAEVKKPGASVKVS | 141 | DIVLTQSPLSLPVTPGESASI | 602 |
| | CKASGYSFSDYYLHWVRQAPG | | SCKSSQSLLHSNGYNYLAWYL | |
| | QGLEWMGWINPNSGDTNYAQK | | QKPGQSPQLLIYLGSHRASGV | |
| | FQGRVTMTRDTSIITAYMELS | | PDRFSGSGSGTDFTLKISRVE | |
| | RLRSNDTAVYYCARSRTWGWH | | AEDVGVYYCMQPLQTPLTFGG | |
| | AEGDFWGQGTRVTVSS | | GTKVEIK | |
| AB-001571 | QVQLVESGGGVVQPGRSLRLS | 142 | EIVMTQSPATLSVSPGERATL | 603 |
| | CAASGFTFSTYGMHWVRQAPG | | SCRASQSVSSNLAWYQQKPGQ | |
| | KGLEWVAIISYDGSNKYYADS | | APRLLIYGASTRATGIPATES | |
| | VKGRFTFSRDNSKNTLYLQMN | | GSGSGTEFTLTISSLQSEDFA | |
| | SLRAEDTAVYYCAKDGVGEYC | | VYYCQQYNNWWTFGQGTKVEI | |
| | SGATCSSGAFDIWGQGTMVTV | | K | |
| | SS | | | |
| AB-001572 | QVQLVESGGGVVQPGRSLRLS | 143 | DIVMTQTPLSLSVTPGQPASI | 604 |
| | CAASGFTFSSYGMHWVRQAPG | | SCKSSQSLLHSDGKTYLYWYL | |
| | KGLEWVAVIWYDGSNKFYADS | | QKPGQSPQLLIYEVSSRFSGV | |
| | VEGRFTISRDNSKNTLYLQMN | | PDRFSGSGSGTDFTLKISRVE | |
| | SLRPEDTAVYYCAKVYFRGGP | | AEDVGVYYCMQGIHLWTFGQG | |
| | GAFDIWGQGTMVTVSS | | TKVEIK | |
| AB-001574 | QVHLVESGGGVVQPGRSLRLS | 144 | DIQMTQSPSTLSASVGDRVTI | 605 |
| | CAASGFTFSNYAMHWVRQAPG | | TCRASQSISSWVAWYQQKPGK | |
| | KGLEWVAVISYHERNKYYADS | | APKFLIYKASILQSGVPSRFS | |
| | VKGRFTISRDNSKNTLFLQMD | | GSGSGTEFTLTISSLQPDDFA | |
| | SLTAADTSVYYCARDGHSSSY | | TYYCQQYNSYSGLTFGGGTKV | |
| | FDYWGQGTLVTVSS | | EIK | |
| AB-007075 | QAQSVESGGGVVQPGRSLRLS | 145 | EIVLTQSPATLSLSPGERATL | 606 |
| | CAVSGFTFRNFGMHWVRQAPG | | SCRASQNVNTYLAWYQQKPGR | |
| | KGLEWVAVIWFDGSQTFYADS | | APRLLIYDASFRATGIPARFS | |
| | VEGRFTVSRDNSKSTLYLQMN | | GSGSGTDFTLTISSLEPEDFA | |
| | SLRVDDTAVYYCARDRGYSGS | | LYYCQQRSSGFTFGPGTKVDI | |
| | WGYFDNWGQGTLVTVSS | | K | |
| AB-007077 | QGQVVESGGGVVQPGRSLRLS | 146 | DIVLTQSPATLSLSPGERATL | 607 |
| | CAASGFTFSNFGMHWVRQAPG | | SCRASQSVGSYLGWYQQKPGQ | |
| | KGLEWVAVIWHDGSNTFYADS | | APRLLIYDASNRATGIPARFS | |
| | VEGRFTISRDNSKNTVSLQMN | | GSGSGTDFTLTISSLEPEDFA | |
| | SLRADDTAMYYCARDRGYSGS | | IYYCQQRSSGFTFGPGTKVDI | |
| | WGYFDHWVQVTMVIVSS | | K | |
| AB-007078 | QVQPVQSGGGVVQPGRSLRLS | 147 | EIVLTQSPATLSLSPGERATL | 608 |
| | CAVSGFTFSNFGMHWVRQAPG | | SCRASQSISSYLAWYQQKRGQ | |
| | KGLEWVGVIWHDGSKKFYADS | | APRLLIYDASNRATGIPARFS | |
| | VEGRFTISRDNSKNTLYLQMN | | GSGSGTDFTLTISSLEPEDFA | |
| | SLRAEDTAVYYCARDRGYGTS | | VYYCQQRSSGFTFGPGTKVDI | |
| | WGYFDFWGQGTLVTVSS | | K | |
| AB-007079 | QVQLVESGGGVVQPGGSLRLS | 148 | EIVLTQSPATLSLSPGERATL | 609 |
| | CVASGFIFSNYGMYWVRQAPG | | SCRASQSVSNYLGWYQQKPGQ | |
| | KGLEWVGVIWEDGSNIFYGDS | | APRLLIYDASVRATGIPARFS | |
| | VEGRFTISRENSKNTLYLQMN | | GSGSGTDFTLTINSLEPEDFA | |
| | SLRAEDTAVYYCARDRGYTGS | | VYYCQQRSIGFTFGPGTKVDV | |
| | WGYFGYWGQGTPVTVSS | | R | |
| AB-007081 | QVQLVESGGGVVQPGRSLRLA | 149 | EIVLTQSPATLSLSPGERATL | 610 |
| | CEASRFIFRNFGMHWVRQAPG | | SCRASQSVTTYLAWYQQKPGQ | |
| | KGLEWVGVIWHDGSNEFYADS | | APRLLIYDASNRATGIPDRFS | |
| | VEGRFTISRDNSKNTLYLQMN | | GSGSGTDFTLSINSLEPEDFA | |
| | NLRVDDTAVYYCARDRGYGGS | | VYYCQQRSKGFTFGPGTKVDF | |
| | WGYFDYWGRGTLVTVSS | | K | |
| AB-007082 | QVQLVEPGGGVVQPGRSLRLS | 150 | EIVLTQSPATLSLSPGERATL | 611 |
| | CEVSTFTFSNFGMHWVRQAPD | | SCRASQSVSSYLGWYQQKPGQ | |
| | RGLEWVAVIWFDGSKKYYADS | | APRLLIYDASNRATGIPARFS | |
| | VEGRFSISRDNSKNTLYLQMN | | GSGSGTDFTLTISSLEPEDFA | |
| | SLRPEDTAVYYCARDRGYSGS | | VYYCQQRSSGFTFGPGTKVDF | |
| | WGYFDHWGQGTLVTVSS | | K | |
| AB-007084 | QVQLVESGGGVVQPGRSLRLS | 151 | GVQMTQSPSTLSASVGDRVTL | 612 |
| | CAASGFPFNTYGMHWVRQTPG | | TCRASQSVSSWLAWYQQKPGK | |
| | KGLEWVAIIWYDGSQKYYADS | | APKLLIYDASRLESGVPSRFS | |
| | VQGRFTISRDNHKDTLSLQMN | | GSGSGTEFTLTISSLQPDDFA | |
| | GLRVEDTAVYFCVRVRFSVGP | | TYYCQQYASYSFWTFGQGTKV | |
| | HGSAFDLWGQGTMVTVSS | | EIK | |
| AB-007085 | QVHLVESGGGVVQPGRSLRLS | 152 | DIQMTQSPSTLSASVGDRVTI | 613 |
| | CAASGFTFSTYGMHWVRQAPG | | TCRASQSISSWLAWYQQKPGK | |
| | KGLEWVAIIWYDGSQKYYAGS | | APKLLIFDASSLENGVPSRFS | |
| | VEGRFTISRDNSKNTLYLQMN | | GSGSGTEFTLTISSLQPDDFA | |
| | SLRAEDTAVYHCARVRYSSSH | | TYFCQQYNSYSFWTFGQGTKV | |
| | HGSAFDLWGQGTMVTVSS | | DIK | |
| AB-007086 | QVQLVESGGGVVQPGRSLRLS | 153 | DIQMTQSPSTLSASVGDRVTI | 614 |
| | CAASGFMFNTYGMHWVRQTPG | | TCRASQSINNWLAWYQLKPGK | |
| | KGLEWVAIIWYDGSYKFYADS | | APKLLIYDASNLQTGVPSRFS | |
| | VEGRFTISRDNSKNTLYLQMN | | GSGSGTEFTLTISSLQPDDFA | |
| | SLRAEDTAVYYCARVRHSRSQ | | TYYCKQYNSYSFWTFGQGTKV | |
| | HGSAFDLWGQGTTVTVSS | | EIK | |
| AB-007089 | QAQLVESGGGVVQPGRSLRLS | 154 | EIVLTQSPATLSLSPGERATL | 615 |
| | CAASGFSFSTYGMHWVRQAPG | | SCRASQSVSSYLAWYQQKPGQ | |
| | KGLEWVAVIWYDGSIKHHAKS | | APRLLIYGATNRATGIPARFS | |
| | VEGRFTISRDNSKNTLYLQMN | | GSGSGTDFTLTINRLEPEDFA | |
| | SLRAEDTAVYYCTRDLDYSNH | | VYYCQQRSSWFTFGQGTRLEI | |
| | NYFDPWGQGTLVTVSS | | K | |
| AB-007091 | QEQLVESGGGVVQPGRSLRLS | 155 | EIVLTQSPATLSLSPGERAIL | 616 |
| | CVASGFSFGSYGMHWVRQAPG | | SCRASQSVSTDLAWYQQKPGQ | |
| | KGLEWVSVIWEDGSNTNYADS | | APRLLIYGASNRVTGIPARFS | |
| | VRGRFTISRDNSRNTVYLKMN | | GSGSGTDFTLTVSSLEPEDFA | |
| | SLRVEDTAVYYCARDFDFYAH | | IYYCQQRSNGFTFGQGTRLEI | |
| | NYFDPWGQGTLVTVSS | | K | |
| AB-007092 | QEQLVESGGGVVQPGRSLRLS | 156 | EIVLTQSPATLSVSPGERATL | 617 |
| | CEASGFSFRSYGMHWVRQAPG | | SCRASQSVSSSLAWYQQKPGQ | |
| | KGLEWVAVIWFDGSNTLYADS | | APRLLIYDASNRATGIPARFS | |
| | VRGRFTVSRDNAKNTLYLQMN | | GSGSGTDFTLTISTLEPEDFA | |
| | NLRAEDTAVYYCAKDLDYENH | | VYYCQQRSNGFTFGQGTRLEI | |
| | NYLDPWGQGTLVTVSS | | K | |
| AB-007093 | QVQLVESGGGVVQPGRSLRLS | 157 | EIVLTQSPATLSLSPGEGATL | 618 |
| | CAASGFDFNNYGMHWVRQAPG | | SCRASQSVGNYLAWYQQKPGQ | |
| | KGLEWVAVIWEDGSQKFHAAS | | APRLLIYDASSRATGIPARFS | |
| | VEGRFTISRDNSKNTLYLQMN | | GSGSGTDFTLTISSLEPEDFA | |
| | SLRAEDTAVYYCARDLDYYHH | | VYYCQQRSNWFTFGQGTRLEI | |
| | NYFGHWGQGTLVTVSS | | K | |
| AB-007094 | QEQLVESGGGVVQPGRSLRLS | 158 | EIVLTQSPATLSLSPGERATL | 619 |
| | CAASGFTFSSYGMHWVRQAPG | | SCRASQSVRTNLAWYQQKPGQ | |
| | KGLEWVAVIWHDGSNAYYADS | | APRLLIYDASNRATGIPARES | |
| | VRGRFTISRDNSKNTLYLQMN | | GSGSGTDFTLTISSLEPEDFA | |
| | SLRAEDTAVYYCARDEDYENH | | VYYCQQRSNWFTFGQGTRLEI | |
| | NYFDPWGQGTLVTVSS | | K | |
| AB-007095 | QVQLVQSGGGVVQPGRSLRLS | 159 | EIVLTQSPATLSLSPGERATL | 620 |
| | CAASGFIFSNYGMHWVRQAPG | | SCRASQSVSTHLAWYQQKPGQ | |
| | KGLEWVAVIWYDGSTEHYAAS | | APRLLIYDASNRATGIPARFS | |
| | VEGRFSVSRDNSKNTLYLQMN | | GSGSGTDFTLTISSLEPEDFA | |
| | SLRVEDTAVYYCARDFDYSNH | | IYYCQQRSQWFTFGQGTRLEI | |
| | NYFGPWGQGALVTVSS | | K | |
| AB-007097 | QVQLVESGGGVVQPGRSLRLS | 160 | EIVLTQSPATLSLSPGERATL | 621 |
| | CEVSGFDFNNYGMHWVRQTPG | | SCRASQSVSSSLAWYQQKPGQ | |
| | KGLEWLAVIWYDGSNKFYADS | | APRLLIYEASNRATGIPPRES | |
| | VKGRFTISRDNSKNTLFLQMN | | GSGSGTDFTLTISSLEPEDFA | |
| | SLRADDTAVYYCARDLDYYNH | | VYYCQQRRNGFTFGQGTRLEM | |
| | NYFHPWGQGTLVTVSS | | K | |
| AB-007098 | QELLVESGGGVVQPGRSLRLS | 161 | QIVLTQSPATLSLSPGERATL | 622 |
| | CAASGFGFSGYGMHWVRQAPG | | SCRASQSVSSSLAWYQQKPGQ | |
| | RGLEWVAVIWYDGSKKYYGDS | | APRLLIYDASNRATGIPARFS | |
| | VKGRFTISRDNSNDTLYLQMN | | GSGSGTDFTLTISSLEPEDFA | |
| | SLRVEDTAVYYCARDFDYFSP | | LYYCQQRSKGFTFGQGTRLEI | |
| | NYFDPWGQGTLVTVSS | | K | |
| AB-007099 | QEQLVESGGGVVQPGRSLTLS | 162 | EIVLTQSPATLSLSPGERATL | 623 |
| | CVASGFSFRSYGMHWVRQAPG | | SCRASQSVGSSLAWYQQKPGQ | |
| | KGLEWLAVIWEDGSNKYYADS | | APRLLMYDTSNRASGIPARES | |
| | VKGRFTISRDNSNNTLYLQMN | | GSGSGTDFTLTISSLEPEDFA | |
| | SLRADDTAVYYCARDADYYKH | | IYYCQQRANWFTFGQGTRLEI | |
| | NYFDPWGQGVLVTVSS | | K | |
| AB-007100 | QVQLVESGGGVVQPGRSLRLS | 163 | EIVLTQSPATLSLSPGERATL | 624 |
| | CAASGFSFSSYGMHWVRQAPG | | SCRASQSVSSYLGWYQQKPGQ | |
| | KGLEWVAIIWYDGSNTYYADS | | APRLLIYDASKRATGIPARFS | |
| | VKGRFTISRDNSKNTLYLQMN | | GSGSGTDFTLTISSLEPEDFA | |
| | SLRAEDTAVYYCAGDHDYFNH | | VYYCQQRSNWFTFGQGTRLEI | |
| | NYFDPWGQGTLVTVSS | | K | |
| AB-007184 | QVQLVESGGGVVQPGRSLRLS | 164 | EIVLTQSPATLSLSPGERATL | 625 |
| | CAASGFSFSNYGMHWVRQAPG | | SCRASQSVTTYVAWYQQKPGQ | |
| | KGLEWMAVIWHDGSNKFYADS | | APRLLIYGASNRATGIPARFS | |
| | LKGRFTISRDNSNNTLYLQMN | | GSGSGTDFTLTISSLEPEDFA | |
| | SLRAEDTAVYYCTRDLDYYNH | | VYYCQQRSQWFTFGQGTRLEI | |
| | NYFDPWGQGTLVTVSS | | K | |
| AB-007105 | QVQLVESGGGVVQPGRSLRLS | 165 | EIVLTQSPATLSLSPGERATL | 626 |
| | CAASGFTFSSQGMHWVRQAPG | | SCRASQSVSSYLAWYQQKPGQ | |
| | KGLEWVAVIWYDGSNKYYADS | | APRLLIYDASNRATGIPARFS | |
| | VKGRFTISRDNSKNTLYLQMN | | GSGSGTDFTLTISSLEPEDFA | |
| | SLRAEDTAVYYCARDGTNWDE | | VYYCQQRSNWPPAFGPGTKVD | |
| | SSGYILDHWGQGTLVTVSS | | IK | |
| AB-007116 | EVQLVESGGGLVQPGRSLRLS | 166 | DIVMTQTPLSLSVTPGQPASI | 627 |
| | CTASGFTFGDYAMSWFRQAPG | | SCKSSESLLNTDGKTYFYWYL | |
| | KGLEWVGFIRITAYGGTPERA | | QKPGQSPQLLIYEVSNRFSGV | |
| | ASVKGRFIFSRDDSKSIAYLQ | | PDRFSGSGSGTDFTLKISRVE | |
| | MSTLKTEDTAVYYCARVRSSD | | AEDVGIYYCMQTIQLPWTFGQ | |
| | FRDMDVWGKGTTVTVSS | | GTKVEIK | |
| AB-007117 | EVQLVESGGGLVQPGRSLRLS | 167 | DIVMTQTPLSLSVTPGQPASI | 628 |
| | CTASGFTFGDYAMSWFRQAPG | | SCKSSQSLLHSDGKTYLYWYL | |
| | KGLEWVGFIRSKAYGGTTEYA | | QKPGQPPQLLIYEVSNRFSGV | |
| | ASVKGRFTISRDDSKSIAYLQ | | PDRFSGSGSGTDFTLKISRVE | |
| | MNSLKTEDTAVYYCTRVRSYD | | AEDVGVYYCMQSIQLPWTFGQ | |
| | FRDMDVWGKGTTVTVSS | | GTKVEIK | |
| AB-007119 | QVQLVESGGGVVQPGRSLRLS | 168 | DIVMTQTPLSLSVTPGQPASI | 629 |
| | CTASGFIFTNYGMHWVRQAPG | | SCKSSQSLLHSDGKTYLYWYL | |
| | KGLEWVAVIWYDGSNEFYADS | | QKPGQSPHLLIYEVSSRFSGV | |
| | VEGRFTISRDNSKNTLYLQMN | | PDRFSGSGSGTDFTLKISRVE | |
| | SLRAEDTAVYYCARSYLSSWL | | AEDVGVYYCMQGIHPRTFGQG | |
| | GFDYWGQGTLVTVSS | | TKVEIK | |
| AB-007122 | QVQLVESGGGVVQPGRSLRLS | 169 | DVVLTQTPLSSPVTLGQPASI | 630 |
| | CAASGFTFSNSGMHWVRQAPG | | SCRSSQSLVYSDGNTYLSWLQ | |
| | KGLEWVALISYDGSNKFYADS | | QRPGQPPRLLIYKIFNRFSGV | |
| | VRGRFTISRDNSKNTLFLQMN | | PDRFSGSGAGTDFTLNISRVE | |
| | SLRAEDTAVYYCATDSLYYYD | | AEDVGVYYCMQATQLYTFGQG | |
| | AHLFDYWGQGTLVTVSP | | TKLEIK | |
| AB-007124 | QVQLVESGGGVVQPGRSLRLS | 170 | DIVMTQTPLSSPVTLGQPASI | 631 |
| | CAASGFIFSSHGMHWVRQAPG | | SCRSSHTLVSSDGNTYLNWLQ | |
| | TGLEWVALTSYDGSNKFYADS | | QRPGQPPRLLIYKISNRFSGV | |
| | VKGRFTISRDNSKNTVYLQMN | | PDRFSGSGAGTDFTLKISRVE | |
| | SLRAEDTAVYYCATDSLDFYN | | AEDVGVYYCMQATQLYTFGQG | |
| | YHLFDYWGQGTLVTVSS | | TKLEIK | |
| AB-007125 | EVQLVESGGGLVQPGGSLRLS | 171 | DVVMTQSPLSLPVTLGQPASI | 632 |
| | CAASGFTFSSYEMNWIRQAPG | | SCRSSQSLVYSDGNTYLNWFQ | |
| | KGLEWVSYISSSGSAKYYADS | | QRPGQSPRRLIYKVSNRDSGV | |
| | VKGRFSISRDNAENSLSLQMN | | PDRFSGSGSGTDFTLKISRVE | |
| | SLRAEDTAVYYCARDHSTSAR | | AEDVGVYYCMQGTHWPYTFGQ | |
| | EYGLDVWGQGTTVTVSS | | GTKLEIK | |
| AB-007127 | EVQLVESGGGLVQPGGSLRLS | 172 | DVVMTQSPLSLPVTLGQPASI | 633 |
| | CVVSGFTFSNYVMNWVRQAPG | | SCRSGQSLVYSDGNTYLYWFQ | |
| | KGMEWVSFISSSGSKTRYADS | | QRPGQSPRRLIYKVSNRDSGV | |
| | VKGRFTISRDNARNALYLQMN | | PDRFSGSGSGTDFTLKISRVE | |
| | SLRDEDTAVYYCARDHPSSHR | | AEDVGVYFCLQGTHWPYTFGQ | |
| | EYGLDVWGQGTTVIVSS | | GTKLEIK | |
| AB-007128 | EVQLVESGGGLVQPGGSLRLS | 173 | DVVMTQSPLSLPVTLGQPASI | 634 |
| | CAASGFTFSSYEMNWVRQAPG | | SCRSSQSLLYTDGNTYLYWFQ | |
| | KGLEWVAYSSRSGTTMLYADS | | QRPGQSPRRLIYKVSNRDSGV | |
| | VKGRFTSSRDNAKNSLYLQMS | | PDRFSGSGSGTDFTLKISRVE | |
| | SLRAEDTAVYYCARDHSTSAS | | AEDVGVYYCMQGTHWPYTFGQ | |
| | EYGLDVWGQGTTVTVSS | | GTKLEIK | |
| AB-007129 | QVHLVESGGGVVQPGRSLRLS | 174 | EIVMTQSPATLSVSPGERATL | 635 |
| | CAASGFTFSNSAMHWVRQAPG | | SCRASQSISSNLAWYQQKPGQ | |
| | KGLEWVALIWYDGSNKFYAHS | | APRLLIYGASTRATGIPARFS | |
| | VEGRFTISRDNSKNTLYLQMN | | GSGSGTEFTLTISSLQSEDFA | |
| | SLRAEDTAVYYCARAGPYDSS | | VYFCQQYNNWYTFGQGTKLEI | |
| | GYLDYWGQGTLVTVSS | | K | |
| AB-007132 | QVQLVESGGGMVQPGRSLRLS | 175 | EIVMTQSPATLSLSPGDRATL | 636 |
| | CLVSGITFSNYGMHWVRQAPG | | SCRASQSVSSNLAWYQQKRGQ | |
| | KGLEWVALIWYDGSNKFYADS | | APRLLIYGASTKATGVPARFS | |
| | VEGRFTISRDNSKNTLYLHMS | | GSGSGTEFTLTISSLQSEDFA | |
| | SLRAEDTAVYYCARAGYYDNS | | VYYCQQYKNWYTFGQGTKLEI | |
| | GYLDHWGQGTLVTVSS | | R | |
| AB-007133 | QVQLVESGGGVVQPGRSLRLS | 176 | EIVMTQSPATLSVSPGERATL | 637 |
| | CAASRFPFSNYGMHWVRQAPG | | SCRASQSVNSKLAWYQQKPGQ | |
| | KRLEWVALIWSDGSNEFYHHS | | APRLLIYGASTRATDIPGRES | |
| | VEGRFTISRDNSKNTLFLQMN | | GSGSGTEFTLTISSLQSEDFA | |
| | SLRAEDTAVYFCARAGRYDNG | | VYYCQQYSNWYTFGQGTKLEI | |
| | GYLDSWGQGTLVTVSS | | K | |
| AB-007134 | QVQLVESGGGVVQPGRSLRLS | 177 | EIVMTQSPATLSVSPGERATL | 638 |
| | CAASGFSFRNSGMHWVRQAPG | | SCRASQSVSSKLAWYQQKPGQ | |
| | KGLEWVALIWHDGSNQFYADS | | APRLLIYGASNRATGIPARES | |
| | VEGRFSISRDNSKNTLYLQMN | | GSGSGTEFTLTISSLQSEDFA | |
| | SLRAEDTAVYYCARAGYYDSS | | VYYCQQYKHYYTFGQGTKLEI | |
| | GYFDSWGQGTLVTVSS | | K | |
| AB-007136 | QVQLVESGGGVVQPGRSLRLS | 178 | EIVMTQTPATLSVSPGERATL | 639 |
| | CEASGFIFSNSGMHWVRQVPG | | SCRASQSVSSNLAWYQQKPGQ | |
| | KGLEWVALIWYDGSNKFYVKS | | APRLLIYGASTRATGIPARFS | |
| | VEGRFTISRDNSKNTLYLQMN | | GSGSGTEFTLTISSLESEDFA | |
| | SLRAEDTAIYFCARAGRSDNG | | IYYCQQYQNWYTFGQGTMLEI | |
| | GYLDYWGQGALVAVSS | | K | |
| AB-007137 | QVHLVESGGGVVQPGRSLRLS | 179 | QIVMTQSPATLSVSPGERATL | 640 |
| | CAASGFIFSNFGMHWVRQAPG | | SCRASQSVSSNLAWYQQKRGQ | |
| | KGLEWVALIWYDGSNKFYAES | | APRLIIFGASTRATGIPARFS | |
| | VDGRFSISRDNSKNTLYLQLN | | GSGSGTEFTLTISNLQSEDFA | |
| | NLRAEDTAVYFCARAGYDDDS | | VYYCQQYNNWYTFGQGTKLEI | |
| | GYLDSWGQGTLVTVSS | | R | |
| AB-007185 | QVQLVESGGGVVQPGGSLRLS | 180 | EIVMTQSPATLSVSPGERATL | 641 |
| | CTASRFTFRNFGMHWVRQAPG | | SCRASRSVGSKLAWYQQKRGQ | |
| | KGLEWLALIWYDGSNEFYADS | | APRLLIYGASTRATGIPARFS | |
| | VEGRFTISRDNSKNTLYLQMN | | GSGSGTEFTLTISSLQSADFA | |
| | GLRAEDTAVYYCARAGQNDDS | | LYYCQQYNQWYTFGQGTKLEI | |
| | GYLDYWGHGSLVTVSS | | R | |
| AB-007186 | QVQLVESGGGVVQPGTSVRLS | 181 | EILLQQSPATLSVSPGERVTL | 642 |
| | CDASGFTFRNYGMHWVRQVPG | | SCRASQSVSNNLAWYQQKPGQ | |
| | KGLEWVALTWYDGSNEFYAES | | APRLLIYGASTRATGIPPRES | |
| | VEGRFTISRDNSKNTLFLQMN | | GSGSGTEFTLTISSLQSEDFA | |
| | SLRAEDTAVYYCARAGYYDNS | | IYYCQQYNRWYTFGQGTRLEI | |
| | GYLDYWGQGTLVTVSS | | K | |
| AB-007140 | QVQLVESGGGLVQPGGSLRLS | 182 | VVVMTQTPLSLSVTPGQPASI | 643 |
| | CAASGFTFSNYWMTWVRQAPG | | SCKSSQSLLHSDGRTYLYWYL | |
| | KGLEWVANINEDGSEKDYVDS | | QKPGQSPQLLMYEVSNRFSGV | |
| | VKGRFTISRDNPKNSLYLHMD | | PDRFSGSGSGTDFALIISRVE | |
| | SVRVEDTAVYYCARYLNSWGQ | | AEDVGVYYCMQSIELPITFGG | |
| | GILVTVSS | | GTKVEIK | |
| AB-007141 | ELKLVESGGGLVQPGGSLRLS | 183 | DNVMTQTPLSLSVTPGQPASI | 644 |
| | CEASGFKFSNYWMSWVRQAPG | | SCKSSQSLLHSNGKTYLYWYL | |
| | KGLEWVANINQEGSQMDYLDS | | QKPGQSPQLLIYEVSNRFSGV | |
| | VKGRFTISRDNAKNLLLLQMN | | PDRFSGSGSGTDFTLKISRVE | |
| | NLRAEDTAVYYCARYLNFWGP | | AEDVGVYYCMQSIHLPLTFGG | |
| | GILVTVSST | | GTKVEIK | |
| AB-007143 | EVQMVESGGGLVKPGGSLRLS | 184 | DIVVTQSPLSLPVTPGEPASI | 645 |
| | CAASGFTFSNAWMSWVRQAPG | | SCRSSQSLLFWNGDNYLDWYL | |
| | KGPEWVGRIRSKTDGGTTDYA | | QKPGQSPQLVIYLGSYRAPGV | |
| | APVKGRFTISRDDSKNTLYLQ | | PDRFSGSGSGTDFTLKISRVE | |
| | MNSLKTEDTGVYYCITDRDFY | | AEDVGVYYCMQSLQTPHTFGQ | |
| | RSGGDWGQGTLVTVSS | | GTKLEIR | |
| AB-007144 | EVHLVESGGGLVKPGGSLRLS | 185 | DIVMTQSPLSLPVTPGEPASI | 646 |
| | CAVSGFTFSNAWMSWVRQAPG | | SCRSSQSLLYSNGDDYLDWYL | |
| | KGLEWVGRIRSKTDGGTIDYA | | QKPGQSPQLLIYLGSNRASGV | |
| | APVIGRFTISRDDSKSTLYLQ | | PDRFSGSGSGTDFTLKISRVE | |
| | MNSLKIEDTAVYYCITDRDFY | | AEDVGVYYCMQTLQTPHTFGQ | |
| | RSGGHWGQGSLVTVSS | | GTKLEIK | |
| AB-007145 | EVQLVESGGGLVEPGGSLRLS | 186 | DIVMTQSPLSLPVTPGEPASI | 647 |
| | CAASGFTFNNAWMSWVRQAPG | | SCRSSQSLLFYNGDNYLDWYL | |
| | KGPEWVGRITSKTDGGTTDYA | | QKPGQSPQLLIYLGSNRASGV | |
| | APVKGRFTISRDDSKDTLYLQ | | PDRFSGSGSGTDFTLKISRVE | |
| | MNRLKTEDTAVYYCITDRDFY | | AEDVGVYYCMQSIQTPHTFGQ | |
| | RSGGSWGQGTPVTVSS | | GTKLEIK | |
| AB-007146 | EVQLEESGGGLVKPGGSLRLS | 187 | DIVMTQSPLSLPVTPGEPASI | 648 |
| | CAASGFTFSKAWMSWVRQAPG | | SCRSSQSLLYSNGDNYLDWYL | |
| | KGPEWVGRIESKGDGGTIEYA | | QKPGQSPQLLIYLGSNRAPGV | |
| | APVKGRFTISRDDSKNMLYLQ | | PDRFSGSGSGTDEKLKISRVE | |
| | MNSLKTEDTAVYYCITDRDFY | | AEDVGVYYCMQALQTPHTFGQ | |
| | RSGGDWGQGTLVTVSS | | GTKLEIK | |
| AB-007147 | DLQLVESGGGLVKPGESLRLS | 188 | DVVMTQSPRSLPVTPGEPASI | 649 |
| | CVGSGFPENNAWMNWVRQAPG | | SCRSSQTLLFYNGDNYLDWYL | |
| | KGPEWVGRITSKNDGGKTDYA | | QKPGQSPQLLIYLGSYRAPGV | |
| | APVKGRFTISRDDSKSALFLH | | PDRFSGSGSGTDFTLKISRVE | |
| | MNRLRTEDTAVYYCITDRDFY | | AEDVGVYYCMQSIQTPHTFGQ | |
| | RSGGSWGQGTPVTVSS | | GTKLEIK | |
| AB-007148 | EVQLVESGGGLVKPGGSLRLS | 189 | DIVMTQSPLSLPVTPGEPASI | 650 |
| | CATSGFTFSNAWMSWVRQAPG | | SCRSSQSLLYSNGDHYLDWYL | |
| | KGLEWVGRMKSKIYGGTRDYG | | QKPGQSPQVLIYLGSNRAPGV | |
| | APVKGRFTISRDDSKDTLYLQ | | PDRFSGSGSGTDFTLKISRVE | |
| | MNSLKTEDTAVYYCITDRDLY | | AEDVGVYYCMQALQTPHTFGQ | |
| | SSGGYWGQGTLVTVSS | | GTKLEIK | |
| AB-007149 | EVQLVESGGGLVKPGGSLRLS | 190 | DIVMTQSPLSLPVTPGEPASI | 651 |
| | CAVSGFIFSDAWMSWVRQAPG | | SCRSSQSLLFRNGDNYLDWYL | |
| | KGPEWVGRITSKSDGGTRDYA | | QKPGQSPQLLIYLGSNRASGV | |
| | APVRGRFTISRDDSRNTLYLQ | | PGRFSASGSGTDFTLKISRVE | |
| | MNSLKTEDTAIYYCITDRDFY | | AEDVGVYFCMQTLQTPHTFGQ | |
| | RSGGNWGQGTLVTVTS | | GTKLQIK | |
| AB-007151 | EVQLVESGGGLVKPGGSLRLS | 191 | DIVMTQSPLSLPVTPGEPASI | 652 |
| | CVASGFSFTNAWMSWVRQAPG | | SCRSSQSLLFRNGDNYLDWYL | |
| | RGPEWVGRIESKTDGGTIEYA | | QKPGQSPQLLIYLGSNRASGV | |
| | APVKGRFTISRDDSKNTLYLQ | | PDRFSGSGSGTDFTLRISRVE | |
| | LNSLKTEDTGVYYCITDRDFY | | AEDVGIYYCMQSLQTPHTFGQ | |
| | RSGGSWGQGTLVTVSS | | GTKLDIK | |
| AB-007152 | DVQLVESGGGLVKPGGSLRVS | 192 | DIVMTQSPLSLPVTPGEPASI | 653 |
| | CAASGFTFGNAWMSWVRQAPG | | SCRSSQSLLFWNGDNYLDWYL | |
| | KGPEWVGRVRSKSDGGTRDYA | | QKPGQSPQLLIYLGSNRASGA | |
| | APVKGRFTISRDDSKNTLYLQ | | PDRFSGSGSGTDFTLKISRVE | |
| | MNNLKTEDTAVYYCITDRDFY | | AEDVGVYYCMQSLQTPHTFGQ | |
| | RSGGYWGQGTLVTVSS | | GTKLEIK | |
| AB-007153 | QVQLVESGGGVVQPGTSLRLS | 193 | DIQMTQSPSTLSASVGDRVTL | 654 |
| | CEVSGFTFSSYGMHWVRQAPG | | TCRASQTINNWVAWYQQKPGK | |
| | KGLAWVAVIWYDGSTQFYADS | | APKLLIYKASSQKSGVPSRFS | |
| | VEGRFTISRDNSKNTLYLQMT | | GSGFGTEFTLTISSLQPDDFA | |
| | SLRVEDTAVYYCARVGMGAQD | | TYYCQQYSLYWTFGQGTKVDI | |
| | DSSGYYDYWGQGTLVTVAS | | K | |
| AB-007155 | QLQLVESGGGVVQPGRSLRLS | 194 | DIQMTQSPSTLSASVGDRVTI | 655 |
| | CVVSGFSFSSYGMHWVRQAPG | | TCRASQSISVWLAWYQQKPGE | |
| | KGPEWVAVIWHDGSKYYHAPS | | APKLLIYKASSLQPGVPSRFS | |
| | VEGRFTISRDNSKNTLYLQMN | | GSGSGTDFTLTISNLQADDFA | |
| | SLRVDDTAVYYCARVGMGDYK | | TYYCQQYNYFWTFGQGTKVEI | |
| | DSSGYYDQWGQGTMVIVSS | | K | |
| AB-007156 | QVQLVESGGGVVQPGRSLRLS | 195 | DIQLTQSPSTLSASVGDRVTI | 656 |
| | CEASGFTFSSYGMHWVRQTPD | | TCRASHDIISWLAWYQQKPGK | |
| | KGLEWVAVIWHDGSKKYYPDS | | APKLLIYKASSLKSGVPSRFS | |
| | VEGRFTVSRDNSRNTLYLQMN | | GSGSGTEFTLTISSLQPDDFA | |
| | SLRAEDTAVYYCARVGMGAQS | | TYYCQQYNTFWTFGQGTKVEV | |
| | DSYGYYDYWGQGTLVTVSS | | K | |
| AB-007157 | QVQLVESGGGVVQPGRSLRLS | 196 | DIQMTQSPSTLSASVGDRVTI | 657 |
| | CAASGFIFRNYGMHWVRQTPG | | TCRASQSISGWLAWYQQKPGK | |
| | RGLEWVAVIWYDGSNKYYADS | | APKLLIYRASSLKSGVPSRFS | |
| | VEGRFTISRDNSKNTLYLQMN | | GSGFGTEFTLTISSLQPDDFA | |
| | SLTAEDAAIYYCARVGMGAQE | | TYYCQQYNFYWTFGQGTKVDV | |
| | DKSGYYDLWGQGTLVTVAS | | K | |
| AB-007162 | LVQLVESGGGVVQPGRSLRLS | 197 | QSVLTQAPSVSGAPGQRVTIS | 658 |
| | CETSGFTFSNFGMHWVRQTPG | | CTGSSSNIGAGYDVHWYQQLP | |
| | KGLEWVAVIWHDGSQKYYGDS | | GTAPKLLIYGNNNRPSGVPDR | |
| | VEGRFTISRDNSKKTLYLQLN | | FSGSKSGTSASLAITGLQAED | |
| | SLRADDTAVYYCARDIRGAFK | | EADYYCQSYDSSLSGYVFGTG | |
| | VTDGGFDYWGQGTLVTVSS | | TKVTVL | |
| AB-007164 | QVQLVESGGGMVQPGRSLRLS | 198 | QFVLTQPPSVSGTPGQRVTIS | 659 |
| | CAASGFTFSNFGMHWVRQAPG | | CTGGSFNTGAGYEVHWYQQLP | |
| | KGLEWVAVIWFDGSQKYYAHF | | GTAPRLLIYGNNNRPSGVPDR | |
| | VKGRFTISRDNSNDMLYLQMS | | FSGSRSGTSASLAITGLQAED | |
| | SLGAEDTAIYYCARDIRGAYR | | ETDYYCQSYDSSLSGYVFGTG | |
| | VTDGGFDYWGQGTLVTVSS | | TKVTVL | |
| AB-007166 | QVQLVESGGGAVQPGRSLRLS | 199 | QSVLTQPPSVSGAPGQRVTIS | 660 |
| | CEASGFVFDNFGMHWVRQAPG | | CSGSGSNIGANYDVHWYQQFP | |
| | KGLEWVAVIWHDGSQKYYGDS | | GTAPQLLIFGDSNRPSGVPDR | |
| | VKGRFTVSRDNAKNTLYLQMN | | FSGSKSGTSASLAITGLQAED | |
| | SLRAEDTAVYYCATDIRGAYK | | EADYYCQSYDSRLSRYVFGTG | |
| | VTDGGFDSWGQGTLVTVSS | | TKVTVL | |
| AB-007167 | QVQLVESGGGAVEPGRSLRLS | 200 | QSMLTQPPSVSGAPGQRVTIS | 661 |
| | CEASGFVFDNFGMHWVRQAPG | | CSGSGSNIGANYDVHWYQQFP | |
| | RGLEWVAVIWHDGSQKYYGDS | | GTAPQLLIFGTINRPSGVPDR | |
| | VKGRFTVSRDNFKNTLYLQMN | | FSGSRSGTSASLAITGLRAED | |
| | SLRAEDTGLYYCATDIRGAYK | | EADYYCQSYDSRLSRYVFGTG | |
| | VTDGGFDLWGQGTLVTVSS | | TKVTVL | |
| AB-007171 | QVQLVESGGGVVPPGRSLRLS | 201 | QSVLTQPPSVSGAPGQRVTIS | 662 |
| | CATSGFTFSNYGMHWVRQAPG | | CTGSSSNIGAGYDVHWYQQLP | |
| | KGLEWVAIIWYDGSKNFYAAS | | GTAPKLLIYGNINRPSGVPDR | |
| | VEGRFTISRDNSKNTLYLQMN | | FSGSKSGTSASLAITGLQAED | |
| | SLRVEDTAVYYCARAAYYDTS | | EADYYCQSYDRRLSGSWVFGG | |
| | GYGDYWGQGTLVTVSS | | GTKLTVL | |
| AB-007172 | QVQLVESGGGVVQPGGSLRLS | 202 | QSVLTQPPSVSGAPGQRVTIS | 663 |
| | CEASGFTFRNYGMHWVRQAPG | | CTGSSSNIGAGYDVHWYQQLP | |
| | KGLEWVAVIWHDGSKENYADS | | GTAPKLLIYGNNNRPSGVPDR | |
| | VEGRFTISRDNSEDALYLEMS | | FSGSKSGTSASLAITGLQAED | |
| | SLRADDTGVYYCARAGYYDTS | | EADYYCQSYDSSLSASWVFGG | |
| | GYGAHWGQGTLVTVSS | | GTRLTVL | |
| AB-007174 | QVQLVESGGGVVQPGRSLRLS | 203 | QSVLTQPPSVSGAPGQTVTIS | 664 |
| | CAASMFTFRNYGMHWVRQAPG | | CTGSSSNIGAGHDVHWYQHLP | |
| | KGLEWVAVIWHDGSREFYADS | | GTAPKIIIYGDSNRPSGVPDR | |
| | VEGRFTISRDNSKNTLYLEMN | | FSGSKTGTSASLAITGLQAED | |
| | SLRAEDTAIYYCARAAYYENS | | EADYYCQSFDRRLSASWVFGG | |
| | GYGDYWGQGTLVTVSS | | GTKLTVL | |
| AB-007177 | QVQLVESGGGVVQPGRSVRLS | 204 | EIVMTQSPATLSVSPGERATL | 665 |
| | CAASGFMFSTYGMHWVRQAPG | | SCRASQSVSSNLAWYQQKPGQ | |
| | RGLEWVALIWYDGSNKYYRDS | | APRLLIYGAFTRATGIPARFS | |
| | VKGRFTISRDNSKEILYLQMN | | GSGSGTEFTLTISSLQSEDFA | |
| | SLRAEDTAVYYCARDGADYED | | VYYCQQYKRWWTFGQGTKVEI | |
| | PSGGAFDIWGLGTMVTVSS | | K | |
| AB-007179 | QVQLVESGGGVVQPGRSLRLS | 205 | YIQMTQSPSTLSASVGDRVTI | 666 |
| | CAASGFTFRNYGMHWVRQAPG | | TCRASQSISSWLAWYQQKPGK | |
| | KGLEWVALIWYDASNEYYLES | | APKLLIYKASTLQSGVPSRFS | |
| | VKGRFTISRDNTKNTLYLQMN | | GSGSGTEFTLTISSLQPDDFA | |
| | DLRAEDTAVYYCARVGYHTNY | | TYYCQQYHSFWTFGQGTKVDI | |
| | YDSSGFLDYWGQGTLVTVSS | | K | |
| AB-007180 | QVQLVESGGGVVQPGRSLRLS | 206 | DIQMTQSPSTLSASVGDRVTI | 667 |
| | CAASGFTFRSYGMHWVRQAPG | | TCRASQSISSWLAWYQQKPGK | |
| | KGLEWVAVIWYDGSNKYYADS | | APKLLIYKASRLESGVPSRFS | |
| | VKGRFTISRDNSKNTLYLQMN | | GSGSGTEFTLTISSLLPDDFA | |
| | SLRAEDTAVYYCARVGDGTTY | | TYYCQQYESYWTFGQGTKVEV | |
| | HDSSGYLDHWGQGTLVTVSS | | K | |
| AB-007181 | QVQLVESGGGVVQPGRSLSLS | 207 | YIQMTQSPSTLSASLGDRVTI | 668 |
| | CAASGFTFSNYGMHWVRQAPG | | TCRASHSISTWLAWYQQKPGK | |
| | KRLEWVAVIWHDGSNKYYAVS | | APKLLIYRTSTLESGVPSRFS | |
| | VKGRFTISRDNSKNTLYLQMN | | GSGSGTEFTLTISSLQPDDFA | |
| | NLRAEDTAVYYCARVGDGTTN | | TYYCQQYSSYWTFGQGTKVEI | |
| | SDSSGYLDYWGQGTLVTVSS | | K | |
| AB-007183 | QVQLVESGGGVVQPGRSLRLS | 208 | QSMLTQPPSVSGAPGQRVTIS | 669 |
| | CAASGFTFNNYGMHWVRQAPG | | CTGSSSNIGAGYDVHWYQQLP | |
| | KGLEWVAVIWYDGSKKYYVDS | | GTAPKLLIYGNNNRPSGVPDR | |
| | VKGRFTISRDNSKNTLYLQMN | | FSGSKSGTSASLAITGLQAED | |
| | SLRAEDTAVYYCVRDGRYRTS | | EADYYCQSYDISLSTSFVFGT | |
| | HQTSTGFFDLWGQGTLVTVSS | | GTKVTVL | |
| AB-001392 | QVQLQESGPGLVRSSETLSLT | 209 | DIQMTQSPSSLSASVGERVII | 670 |
| | CTVSGDSMRRYFRNWIRQSPG | | TCQASQDINHYLNWYQQKPGT | |
| | RGLEWIGAIFDNGSTRYNPSL | | APKLLIYDASKLETGVPSRFS | |
| | KSRVTMSVDVSKNQFSLRLSS | | GSGSGTDFTFTIASLQPEDFA | |
| | VTAADTAVYYCARARDYYDEL | | TYYCQHYDLYPLFGGGTKVDV | |
| | TRTYRNFYFDYWGQGTLVTVS | | K | |
| | S | | | |
| AB-001393 | EVQLLESGGGLVQPGGSLRLS | 210 | DIQMTQSPSSLSASVGDRVTI | 671 |
| | CAASGFTFSNHAMSWVRQAPG | | TCQASQDISNYLIWYQQKPGK | |
| | KGLQWVSAISGSGDRTYYADS | | APKLLIYDASNLETGVPSRFS | |
| | VKGRFTISRDNSKNTLYLQMN | | GSGSGTHFTFTISSLQPEDIA | |
| | SLRADDTAVYYCAKDQGGYSI | | TYYCQQFDNLPLTFGGGTKVE | |
| | YPLGMDVWGQGTTVTVSS | | IK | |
| AB-001437 | EVQLVESGGGLVQPGGSLRLS | 211 | QSVLTQPPSVSAAPGQKVTIS | 672 |
| | CAASGFTFRHYAMNWVRQAPG | | CSGSSSNIGYDYVSWYRQLPG | |
| | KGLEWVSSISGSGGNTYYAVS | | TAPKLLIYDNDKRPSGIPDRF | |
| | VKGRFTISRDNSENTLYLQMN | | SGSKSGTSATLGITGLQTGDE | |
| | SLRAADTAVYYCARSLDYGGN | | AVYYCGTWDSSLSAGVFGGGT | |
| | SLSPEYWGQGTLVIVSS | | KLTVL | |
| AB-001502 | EVHLVESGGGLVKPGGSLRLS | 212 | SYVLTQPPSVSVAPGETATIT | 673 |
| | CAASGFTFSSYTMTWVRQAPG | | CGGNNIGSKSVHWYQQRPGQA | |
| | KGLEWVSSISSTSTHIYYADS | | PVLVIYYDSNRPSGIPERLSG | |
| | VRGRFTISRDNAKNSLYLQIN | | SNSGNTATLTITWVEAGDEAD | |
| | SLGAEDTGLYYCARIFGIASA | | YYCQVWDSYTAVFGGGTKLTV | |
| | DTTFDQWGQGTLVSVSS | | L | |
| AB-001525 | QVQLVESGGGLVKAGGSLRLS | 213 | SYSLTQPPSVSVSPGQTASIT | 674 |
| | CAASGFAFKDYYMSWIRQAPG | | CSGDKLGDKYVSWYQQKPGQS | |
| | KGLEWISYISGRGSTIYYADS | | PVLVIYKDNKRPSVIPERFSG | |
| | AKGRFTISRDNAKNSLYLQMS | | SNSGNTATLSISGTQALDEAD | |
| | SLRAEDSAIYYCAGGGGYPQL | | YYCQAWDSSAVVFGGGTKLTV | |
| | THSYYMDVWGKGTTVTVSS | | L | |
| AB-001534 | AVQLVESGGGLVKPGGSLRLS | 214 | EIVLTQSPGTLSLSPGERATL | 675 |
| | CAASGFTFSLYSMTWVRQAPG | | SCRASQSLNSNYLAWYQQKPG | |
| | KGLEWVSSISTSSSYIYYADS | | QAPRLLIYGASSRATGIPDRF | |
| | VKGRFTISRDNAKKTLYLHMN | | SGSGSGTDFTLTISRLEPEDF | |
| | SLRAEDTAVYYCARDRGTALR | | AVYYCQQYGNSHTFGQGTKVE | |
| | NFEWSDASDIWGQGTKVTVSS | | IK | |
| AB-000307 | QVQLVESGGAVVQPGKSLRLS | 215 | DIQMTQSPSSLSASIGDRVTI | 676 |
| | CAASGFTFSNYAVHWVRQAPG | | TCRASQTISSSLSWYQQRPGK | |
| | KGLEWVAFISYDSRNKYYADS | | APRLLIYSASSLQRGVPSRFS | |
| | VKGRFTISRDNSKNTLYLHMD | | GSGSGTDFTLTITRLQPEDFA | |
| | SLRPEDTAVYYCARDLLVGVS | | TYYCQQSYSTPFTFGPGTTVD | |
| | QYWGQGTLVTVSS | | MK | |
| AB-001402 | QMQLVQSGPEMKKPGSSVKVS | 216 | SYELTQPPSVSLSPGQTARIT | 677 |
| | CKAPRGTFSSFTIGWVRQAPG | | CSADVLSKQYAYWYQQKPGQA | |
| | HGLEWVGMIVPLFGIPKYAPK | | PVLVMSKDTERPSGIPERFSG | |
| | LQGRVTISADDSTDTAYMELS | | SRSGTTVTLTISGVQAEDEAD | |
| | SLRSEDTAIYYCARDRYDWNE | | YYCQSMDSDPAYVLIFGGGTK | |
| | RVWYFDLWGRGTLVTVSS | | LTVL | |
| AB-001404 | QVQLVESGGGVVQPGRSLRLS | 217 | DIQMTQSPSTLSASVGDRVTI | 678 |
| | CAASGFTFSSYGMHWVRQAPG | | TCRASQSISSWLAWYQQKPGK | |
| | KGLEWVAVIWYDGSNKYYADS | | APKLLIYKASSLESGVPSRFS | |
| | VKGRFTISRDNSKNTLYLQMN | | GSGSGTEFTLTISSLQPDDFA | |
| | SLRAEDTAVYYCARVGYGGHY | | TYYCQQYNSYWTFGQGTKVEI | |
| | YDSSGYYDYWGQGTLVTVSS | | K | |
| AB-001407 | QEQLVESGGGVVQPGRSLRLS | 218 | EIVMTQSPATLSVSPGERATL | 679 |
| | CVASGFIFRNFGMHWVRQAPG | | SCRASQRVNSDLAWYQQKPGQ | |
| | KGLEWVAVIWYDGSYEFYADS | | PPRLLIYGASTRATGIPARFS | |
| | VKGRFTISRDNSKNTLYLQMN | | GSGSGTEFTLTISSLQSEDFA | |
| | SLRANDTAVYYCASDRRNPGI | | VYYCQQYNNWFTFGLGTKLEI | |
| | GAAVGAFDIWGQGTMVTVSS | | K | |
| AB-001415 | EVQLVESGGGLVKPGGSLRLS | 219 | SYVLTQAPSVSVAPGKTATIT | 680 |
| | CAASGFTFSSYTMNWVRQAPG | | CGGNNIGSKSVYWYQQRPGQA | |
| | KGLEWVSCITSSSSYIYYADS | | PVLVVFDDSDRPSGIPERFSG | |
| | VKGRFTISRDNAKNSLYLQMD | | SNSGNTATLSISRVEAGDEAD | |
| | SLRAEDTAVYYCARVVGFCVG | | YYCQVWDTTTDHPGVFGGGTK | |
| | GSCYSDYWGQGTLVTVSS | | LTVLS | |
| AB-001425 | QVQLVESGGGVVQPGRSLRLS | 220 | DIVMTQSPDSLAVSLGERATI | 681 |
| | CAASGFTFSSYAMHWVRQAPG | | NCKSSQSVLYSSNNKNYLAWY | |
| | KGLEWVAVISYDGSNKYYADS | | QQKPGQPPKLLIYWASTRESG | |
| | VKGRLTISRDNSKNTLYLQMN | | VPDRFSGSGSGTDFTLTISSL | |
| | SLRAEDTAVYYCARQGGYCTN | | QAEDVAVYYCQQYYSTPITFG | |
| | GVCSEGANYYNYYGMDVWGQG | | QGTRLEIK | |
| | TTVTVSS | | | |
| AB-001435 | QVQLVESGGGVVQPGRSLRLS | 221 | DIQMTQSPSTLSASVGDRVTI | 682 |
| | CAASGFTFSNYGMHWVRQAPG | | TCRASQSISSWLAWYQQKPGK | |
| | KGLEWVAVIWYDGSNKYYADS | | APKLLIFKTSSLESGVPSRFS | |
| | VKGRFTISRDNSKNTLYLQMN | | GSGSGTEFTLTISSLQPDDFA | |
| | SLRAEDTAVYYCARPQGLYGS | | TYYCQQYNSLFTFGPGTKVDI | |
| | TNYVTFDIWGQGTMVTVSS | | K | |
| AB-001441 | QVQLQESGPGLVKPSGTLSLT | 222 | EIVLTQSPGTLSLSPGERATL | 83 |
| | CAVSGGSISSSNWWNWVRQPP | | SCRASQSVSSNYLAWYQQKPG | |
| | GKGLEWIGEIYHSGSPNYNPS | | QAPRLLIYGASTRATGIPDRF | |
| | LKSQVTISVDKSNNQLSLKLT | | SGSGSGTDFTLTISRLEPEDF | |
| | SVTAADTAVYYCASKHYSLFL | | AVYYCQQYGSSPYTFGQGTKL | |
| | GWVVSWGQGTLVTVSS | | DIK | |
| AB-000308 | QVQLVESGGGVVQPGRSLRLS | 223 | QSVLTQPPSVSGAPGQRVTIS | 684 |
| | CAASGFVFSNFGMHWVRQAPG | | CTGSSSNIGAGYDVAWYQQLP | |
| | KGLEWVAVIWHDGSKKYYTES | | GTAPKLLIYSNNNRPSGVPDR | |
| | VEGRFTISRDNSKRTLYLQMN | | FSGSKSGTSVSLAITGLQAED | |
| | SLTAEDTAVYYCVRDGRTVGA | | EADYYCQSYDSSLTIFVFGTG | |
| | TTGWFDPWGQGTLVTVSS | | TKVTVL | |
| AB-000332 | QVHLVESGGGVVQPGGSLRLS | 224 | DVVLTQSPLSLPVTLGQPASI | 685 |
| | CAASGFTFRNYGMHWLRQAPG | | SCRSSHSLVYTDGNTYLNWFQ | |
| | KGLEWVAFIPYDSRDKYYTDS | | QRPGQSPRRLIYKVSNRDFGV | |
| | ARGRFTISKDNSKNTLYLQMN | | PDRFSGSGSGTDFTLKISRVE | |
| | SLRVEDTAVYYCAKVDIAVAG | | AEDVGVYYCMQGAHWPPTFGG | |
| | NLDYWGQGTLVTVSS | | GTKVEIK | |
| AB-000390 | EVQLLESGGGLVQPGGSLRLS | 225 | DIPMTQSPSTLSASVGDRVTI | 686 |
| | CATSGFIFTNYGMTWVRQAPG | | TCRASQNINSWLAWYQQKPGK | |
| | KGLEWVSAISGGGDSTYYADS | | APKLLIYKASSLESGVPTRES | |
| | VQGRFTVSRDNSKNTLYLQMN | | GSGSGTEFTLTISSLQPDDFA | |
| | SLRAEDTAVYYCAKEGGFCSS | | TYYCQQYNFYWSFGQGTKVEI | |
| | ATCYFYMDIWGKGTTVTISS | | K | |
| AB-001399 | QVQLQESGPGLVKPSQTLSLT | 226 | DIQMTQSPSTLSASVGDRVTI | 687 |
| | CTVSSDSISRGGYFWSWIRQH | | TCRASQSISSWLAWYQQKPGK | |
| | PGKGLEWIGYIHYSGSTYYNP | | APKLLIYKASSLESGVPSRFS | |
| | SLKSRVTISVDTSKNQFSLKL | | GSGSGTEFTLTISSLQPDDFA | |
| | SSVTAADTAVYYCARDRGEYC | | TYYCQQYNSYSPYTFGQGTKL | |
| | SGSSCSSYYYYYMDVWGKGTT | | EIK | |
| | VTVSS | | | |
| AB-000355 | DVELVESGGGLVQPGGSLRLS | 227 | QSALTQPASVSGSPGQSITIS | 688 |
| | CAASGFTLSAYWMHWVRQVPG | | CTVTSNYDLVSWYQQHPGKAP | |
| | KGLVWVSRINSDGSYTTYVDS | | KLIIYAVSNRPSGISHRFSGS | |
| | VRGRFTISRDNAKNTVYLQMD | | KSGNTASLTISGLQAEDEADY | |
| | NLRADDTAIYYCVRPDVDNWN | | YCSSYLISNARVFGTGTEVTV | |
| | DEGAFQYWGQGTLVTVSS | | L | |
| AB-001457 | EVQLVESGGGLVKPGGSLRLS | 228 | EIVLTQSPATLSLSPGERATL | 689 |
| | CAASGFTFSYYGMNWVRLTPG | | SCRASQSVSSRYLAWYQQKPG | |
| | KGLDWVSSITSSSGYIYYADS | | QAPRLLIYGASNRATGIPDRF | |
| | VKGRFTISRDNAKNSLYLQMN | | SGSGSGTDFTLTISRLEPEDE | |
| | SLRAEDTAVYYCARDYGSGTY | | AVYYCQQYGSGITFGQGTRLE | |
| | GDYDAFDIWGQGTMVTVSS | | IK | |
| AB-001469 | QVQLQESGPGLVKPSETLSLT | 229 | DIQMTQSPSSLSASVGDRVTI | 690 |
| | CSVSGGSIGTYYWNWIRQSPG | | TCRASQTINSYLNWYQQQPGR | |
| | KGLEWIAYIFYTGRTTYNPSL | | APKLLIYAASRLQSGVPSRFS | |
| | ESRATISLDMSNSQFSLKLSS | | GSGSGTDFTLTITNLQVDDFA | |
| | MTAADTAVYYCARVDLRRDGY | | TYYCQQSYSPPFTFGQGAKLD | |
| | KFDYWGQGTLVTVSS | | IK | |
| AB-001477 | QLQLQESGPGLVKPSETLSLT | 230 | DIQMTQSPSSLSASVGDRVTI | 691 |
| | CAVSGGSIINSRYYWGWIRQP | | TCQASQDISNYLNWYQQKPGK | |
| | PGKGLEWIGSIFYSGSTYYNA | | APKLLIYDASNLETGVPSRFS | |
| | SLKRRVTISVDTSKNQFSLKL | | GSGSGTDFTFTINSLQPEDIA | |
| | SSVTAADTAVYYCARLRVGRY | | TYYCQQYDNLPPTFGQGTKLE | |
| | SSDWLFDYWGQGTLVTVSS | | IK | |
| AB-001543 | EVQLVESGGGLVQPGGSLRLS | 231 | EIVLTQSPGTLSLSPGERATL | 692 |
| | CVVSGFTFSSYEMNWVRQAPG | | SCRASQSVRSSYLAWYQQKPG | |
| | KGLEWVSYISSSGSTIYYADS | | QAPRLLIYGASSRATGIPDRF | |
| | VKGRFTISRDNAKNSLYLQMN | | SGSGSGTDFTLTISRLEPEDF | |
| | SLRAEDTAVYYCAREDSFWSG | | AVYYCQQYGRSTRTFGQGTKV | |
| | YVTRREDYYCMDVWGKGTTVT | | EIK | |
| | VSP | | | |
| AB-000212 | EVQLVESGGGLVQPGGSLKLS | 232 | QAGLTQPPSVSRGLRQTATLT | 693 |
| | CAASGFAFSASAMHWVRQAPG | | CTGNSDNVGYEGAAWLQQHQG | |
| | KGLEWVGRVRSKTNNYATAYS | | HPPKLLSYRNNNRPSGISARF | |
| | ASVEGRETISRDDSKNTAYLQ | | SASWSGNTASLTITGLQPEDE | |
| | MSSLKTDDTAVYYCARSDMAA | | ADYYCSAWDRSLTGWLFGGGT | |
| | PWVAFDIWGQGTMVTVSS | | KLTVL | |
| AB-000213 | EVQLVESGGGLVQPGGSLKLS | 233 | QAGLTQPPSVSKGLRQTATLT | 694 |
| | CAASGFIFSGSGVHWVRQASG | | CTGNSNNVGNEGAAWLQQHQG | |
| | RGLEWVGRSRSKVDRYATAYA | | HPPKLLFYRNNNRPSGISERF | |
| | ASVKGRFIISRDDSSNTAYLE | | SASRSGNTASLTITGLQPEDE | |
| | MNSLKTEDTAVYYCSTQTGTT | | ADYYCSAWDNSLSVWVLGGGT | |
| | GTSANFDLWGQGTLVTVSS | | KVTVL | |
| AB-000214 | QITLKESGPTLVKPTQTLTLT | 234 | QAVLTQPSSLSASPGASASLA | 695 |
| | CTFSGFSLNSFGVGVAWIRQP | | CTLRSDINVRTNMVYWYRQKP | |
| | PGRDLEWLALIYWDDDKRYSP | | GSPPQYLLRYKTDSDKQQGSG | |
| | SLENRLTITKDTSKNQVVLTM | | VPSRFSGSRDDSANAGILLIS | |
| | THMAPADAATYYCAHRTASEQ | | GLQSEDEGDYFCMIYYARAYV | |
| | LDYWGQGAPVTVSS | | FGAGTRLTVL | |
| AB-000215 | EVQLVESGGGLVQPGGPLKLS | 235 | DIVMTQSPDSLAVSLGERAII | 696 |
| | CVASGFSFSDSAIHWVRQAPG | | RCKSSQTLLYTYNNKDYITWY | |
| | KGLEWLGRMRSKTNNDATVYA | | QQKPGQPPRLLIYWASTRESG | |
| | ASVQGRFTISRDDSRNTAFLQ | | VPDRFSGSGSGTDFTLTISSL | |
| | MNSLKTEDTAVYYCTRGEFCG | | QAEDVATYYCQQFSRTPYTFG | |
| | VDCYRDAFDVWGQGTVVTVSS | | QGTKLEIR | |
| AB-000216 | EVQLVESGGGLVQPGGSLRLS | 236 | EAGLTQPPSVSRALRQTATLT | 697 |
| | CSASGFRFTSYWMSWLRQAPG | | CSGNGNNVGNEGAAWMQQHLG | |
| | KGLEWVANIKEDGSVEHYVDS | | HPPKLLSDRSHNRPSGISERF | |
| | VRGRFTISRDNARNSLYLQMS | | SASRSGNTASLTISGLQPEDE | |
| | SLRAEDTAMYYCATASDSCAN | | ADYYCTAWDNSINGWIFGGGT | |
| | DWGQGTLVTVSS | | KLTVL | |
| AB-000217 | EVQLVESGGALVQPGGSLKLS | 237 | DIVMTQSPDSLALSLGERATI | 698 |
| | CAASGYTFSGSGIHWVRQPFG | | NCRSSQSVLYSSNRKHNLAWY | |
| | KGLEWIGHIKNKANNYATDYA | | QQRPGHPPTLLIYWASTRQSG | |
| | ASVNGRFTISRDDSKNTAFLQ | | VPDRFSGRGSGTNFTLTISAL | |
| | MNNLKTEDSAVYFCARPLSGY | | QAEDVGVYYCQQYSIVPWTFG | |
| | DYWGQGTLVTVSS | | QGTKVEIK | |
| AB-000218 | QVQLVESGGGVVQPGRSLRLS | 238 | QAVLTQPSSLSASPGASASLT | 699 |
| | CSASGFTLRRHGMHWVRQAPG | | CTLRSGINVGAYRIYWYHQKP | |
| | KGLEWVASISYGEENKYYLDS | | GSPPQYLLRYKSDSDKHQGSG | |
| | VKGRFTISRDISKNTLHLQMS | | VPRRESGSKDASANAGILLIS | |
| | SVRTEDTAVYFCAKDNYDSLS | | GLQSEDDGDYYCLIWHNSAWV | |
| | GYSTDYNFHGMDVWGQGTTVT | | FGGGTKLTVL | |
| | VSS | | | |
| AB-000219 | QVLLQESGPGLVRPSETLSLT | 239 | QSVLTQPPSVSGAPGQRVTIS | 700 |
| | CTVSGGSISRTSHYWGWIRQS | | CTGSSSNIGAGYDVHWYQQLP | |
| | PGKGLEWIGSAYYSGSTYYNP | | RTAPKLLIYGNNNRPSGVPDR | |
| | SLKSRLTISVDTSKNQFSLRL | | FSGSKSGTSASLAVTGLQPED | |
| | SSVTAADTAVYYCARHTYDIS | | EANYYCQSYDSSLSGSVFGGG | |
| | TGWALFYYYYHIDVWGKGTTV | | TKLTVL | |
| | TVSS | | | |
| AB-000220 | EVQLVESGGGLIQPGGSLRLS | 240 | QTVVTQEPSFSVSPGETVTLT | 701 |
| | CKPSGFTFSYYSMNWVRQAPG | | CGLTSGSVSTSSYASWYQQAP | |
| | KGLEWVSNIGTTGSPRYYSDS | | GQAPRTLIVTTNTRSSGVPDR | |
| | VKGRFTISRDNTNNLLYLQMN | | FSGSILGDKAALTITGARADD | |
| | SLRAEDTALYYCGRAQPGYSN | | ESDYYCLLYVGGGSWVFGGGT | |
| | GWYSAPMVAIDLWGRGTMVTV | | KLTVL | |
| | SS | | | |
| AB-000221 | EVQLLESGGGLVQPGGSLRLS | 241 | DIVMTQTPLSLSVAPGEPASI | 702 |
| | CAASGFTFSNYAMSWVRQAPG | | SCRSSQSLLDSDDGNTYLDWY | |
| | KGLEWVSIISGSGDSTNYADS | | LQRPGQSPHRLIYTLSYRASG | |
| | VKGRFTISRDNSKKKLYLQMN | | VPDRFSGNGSGTDFTLKISGF | |
| | SLRAEDTAVYYCAKVVPEYTS | | EADDVGVYFCMQRIKFPLTFG | |
| | GWYSYYYGYMDVWGKGTTVSV | | GGTKVGIK | |
| | SS | | | |
| AB-000223 | QVHLVQSGAEVKRSGSSVKVS | 242 | EILLTQSPGTLSLSPGERATL | 703 |
| | CMASPGTENSYTISWVRQAPG | | SCRASETISSDYLAWYLQRPG | |
| | QGLEWVGRIIPIFGTTNYAPR | | QPPRLLIFGASSRATDIPDRF | |
| | FKGRVTITADKSTSTAYMELS | | SGSGSGTDFTLTISRLEPEDF | |
| | SLKSEDTAVYYCATMRYYYDR | | AVYYCQQYGTTPYTFGQGTKL | |
| | GGPRGFDSWGQGTLVTVSS | | EIK | |
| AB-000225 | EVQLVESGGNLVQPGGSLRLS | 243 | QSVLTQPPSVSGAPGQRVTIS | 704 |
| | CAGSGFTFSRHWMHWVRQAPG | | CTGSSSNIGAGYNVHWYQHLP | |
| | KGLVWVSRVSTDGSSTSYADS | | GKAPKLLISENSNRPSGVPDR | |
| | VKGRFTISKDNARNTMYLQMN | | FSGSKSGTSASLVITGVQAED | |
| | SLRAEDTAVYYCVRGGTFYFG | | EGDYYCQSYGSSLTAFYVFGT | |
| | DYNYGMDVWGQGTTVTVSS | | GTKVTVL | |
| AB-000226 | EVQLVESGGGLVQPGGSLRLS | 244 | QSALTQPPSVSGAPGQRVTIS | 705 |
| | CAASGFTESSYWMHWVRQAPG | | CTGTSSNIGAHYDVHWYQVVP | |
| | KGLVWVSRLNSNGITTSYADS | | GAAPKLLIYDNSNRPSGVPDR | |
| | VKGRFTISRDNAKNTLYLQMN | | FSGSKSGSSASLAIAGLQPED | |
| | SLRDEDTGVYYCVKGGSGWFF | | EADYYCQSYENSLSGFYVEGT | |
| | DWYFDLWGRGTLVTVSS | | GTKVTVL | |
| AB-000228 | AEHLLESGGGLGQPGGSLRLS | 245 | QSVLTQPPSVSGAPGQRVTIS | 706 |
| | CAASGFSFDDYAMSWVRQAPG | | CTGSSSNIGAGFYVHWYQQLP | |
| | KGPEWVSAIFGSGGGTYYADA | | GTAPKLLIYADTNRPSGVPAR | |
| | VKGRFTISRDNSKSTLYLQMN | | FSGSKSGTSAFLAITGLQAED | |
| | SLRPEDTAVYYCAKEPEGGKG | | EADYYCQSHDSSFDGGVFGGG | |
| | GWLDPWGQGTLVTVSS | | TRLTVL | |
| AB-000229 | QVQLVESGGGVVQPGRSLRLS | 246 | DIQLTQSPSFVSASVGDRVTI | 707 |
| | CAASGFTFSNYGMHWVRQAPG | | TCRASQGIGNYLAWYQQRPGK | |
| | KGLEWVAVIWEDGSYKNTADS | | APRLLIYAASTLQSGVPSRFG | |
| | MRGRFTISRDNSRNTLYLQMN | | GSGSGTEFTLTINSLQPEDFA | |
| | SLRVEDTAVYYCARERSHSSG | | SYYCQQPSGYVWTFGQGTKVE | |
| | WYMGGYGMDVWGQGTTVTVLS | | IK | |
| AB-000230 | QVQLQESGPGLVKPSETLSLT | 247 | QSVLTQPPSVSGAPGQSVTIS | 708 |
| | CTVSGVSVGTYYWSWVRQPPG | | CTGDSSNIGASFDVHWYQQLP | |
| | KALEWMGYIHHRGSSSYSPSL | | GTAPKLLIYGNNNRASGVPDR | |
| | KSRVTLSVDTSKNQVSLSLHS | | FFGSKSGTSASLAITGLQAED | |
| | VTAADTAVYFCARSHPQFSCS | | EADYYCQAYDSSPRAWVFGGG | |
| | SGVCNASSPAEYYGMDVWGQG | | TTLTVL | |
| | TTVTVSS | | | |
| AB-000232 | EVQILESGGGLAQPGGSLRLS | 248 | DIVLTQAPLSSPVTLGQPASV | 709 |
| | CAASGFTFNYFAMSWVRQAPG | | SCRSSQSLVHSDGNTYLSWLQ | |
| | KGLEWVSAIHPSGGATYYADS | | QRPGQPPRLLIYKISNRFSGV | |
| | VKGRFTISRDNSRNTLYLQMN | | PDKFSGSGAGTDFTLKISRVE | |
| | SLRAEDTALYYCTKLDPGYSS | | AEDVGVYYCMQTTQFPLTFGG | |
| | GWYSNAYAHADYWGQGTQVIV | | GTRVEIK | |
| | SS | | | |
| AB-000233 | EVQLVESGGGLVKPGGSLRLS | 249 | DIQMTQSPSTLSASVGDRVTI | 710 |
| | CAASGFSLSTAWMNWVRRAPG | | TCRASQSVSTWLAWYQQKPGQ | |
| | KGLEWVGRIKIKTDGGTADYA | | APNLLIYKASTLKPGVPSRFS | |
| | APVKGRFTISRDDSKNRVFLQ | | GSGSGTEFTLTISSLQPDDSA | |
| | MNSLKTEDTGLYYCTTDIYYY | | TYHCQQYSNDVYTFGQGTRLE | |
| | DFWSYYGMDVWGQGTTVTVSS | | IK | |
| AB-000235 | EVQLVESGGGLVQPGGSLRLS | 250 | QSVLTQPPSVSGAPGQRVTIS | 711 |
| | CAASGFTFSSYWMHWVRQAPG | | CSGISSSYDVHWYQQLPGTAP | |
| | KGLEWVSRISSGGISTSYADS | | KLLIYGNSNRPSGVPDRFSGS | |
| | VKGRFTISGDSAKNTVYLQMN | | KSGTSASLAITGLQAEDEADY | |
| | SLRAEDAAVYYCARERPNLLW | | YCQSYGSSLNAFYVFGTGTKV | |
| | FGEYYFDYWGQGTLVTVSS | | SVL | |
| AB-000237 | QVQLVQSGAEVKKPGASVKVS | 251 | DIVMTQSPLSLPVTPGESASI | 712 |
| | CKTSGYTFTTFAMHWLRQAPG | | SCRSSRSLLYSDGHNYVDWYL | |
| | QRLEWMGWINAGNGLTKYSQK | | QKPGQSPQLLIYLGSNRASGV | |
| | FQGRVTFTSDTSASTVYLELR | | PDRFSGSGSGTDFTLKISRVE | |
| | SLRSEDTAVYFCGLRGGKDAW | | AEDVGVYYCMQALQTPLTFGG | |
| | GQGTLVTVSS | | GTKVEIK | |
| AB-000238 | QVQLVESGGGVVQPGTSLRLS | 252 | NFMLTQPPSVSESPGKTVTIS | 713 |
| | CAASGFTFSSYAIHWVRQAPG | | CIRSSGSIASNYVQWYQQRPG | |
| | EGLEWLAVMSFDRSNKYADSV | | RAPTTVIYENNRRPSGVPDRF | |
| | KGRFIISRDDSKNTVFLQMNR | | SGSIDSSSNSASLTISGLQTE | |
| | LTSEDTAVYYCAKDVTADLNW | | DEAAYYCQSYVPRIQAVLGGG | |
| | FLDFWGRGTLVTVSS | | TLLTVL | |
| AB-000241 | QVQLHQSGPGLVQPSQTLSLT | 253 | DIVMTQSPLSLSVTPGEAASI | 714 |
| | CVISGDSISSNSIGWTWIRQS | | SCRSSQSLLNSNGNTYLDWYL | |
| | PSRGLEWLGRIFYRSKWSTDY | | QKPGQSPQLLISLASNRASGV | |
| | TDSVRGRMVIIPDASKNQLSL | | PDRFSGSGSGTDFILRIWRVE | |
| | QLNSVTPEDTAVYYCARSRLL | | PEDVGVYYCMQALQAPFSFGQ | |
| | GRGLDYWGQGILVTVSS | | GTKLEVK | |
| AB-000242 | EVQLVESGGGLVQPGGSLRLS | 254 | SYELTQPPSVSVSPGHTARIT | 715 |
| | CAASGFTFSTSWMSWVRQAPG | | CSGDALPKQYAYWYQQKSGQA | |
| | KGLEWVANINQDGTEKYYGDS | | PVLVIYEDSKRPPGIPERFSG | |
| | VKGRFIISRDNAKNSLYLQMK | | SSSGATVTLTISGVQAEDEAD | |
| | SVRAEDTAVYYCVAGSSPWGQ | | YYCQSADSSTSYVVFGGGTKL | |
| | GALVTVSS | | TVL | |
| AB-000243 | EVQLLESGGGLVQPGGSLTVS | 255 | EVVMTQSPATLSVSPGGRATL | 716 |
| | CAASGFTFSRYAMSWVRLAPG | | SCRASPGINNNLAWYQLKPGQ | |
| | KGLEWVSAISGSGETVYYADS | | APRLLIYDASTRASGVPARES | |
| | VKGRFTVSRDNSKSTLFLQMH | | GSGSGTEFTLTITSLQSEDFA | |
| | SLRGDDTALYYCARFPGVFKW | | LYFCQQYNNWPPYTFGQGTTL | |
| | FGDAFDLWGRGTRVTVSA | | DIK | |
| AB-000244 | EVQLLESGGDLVEIGGSLRLS | 256 | SSKLTQDPAVTVALGQTVRIT | 717 |
| | CAASGFSFSGYAMSWVRQAPG | | CQGDSLNDNFATWFQQKPGQA | |
| | QGLEWVAIIYSDGTSTFYADS | | PVLVIFGQNSRPSGIPGRESA | |
| | VEGRFIISRDNSAETLFLQMN | | SNSGNTASLTVTGAQAEDEAV | |
| | RLTVEDTAIYFCARGFYFDSW | | YYCNSRDNSEYVWLFGGGTKL | |
| | GQGTLVAVSS | | TVLS | |
| AB-000247 | QVQLQESGPGLVKPSETLSLT | 257 | EIVLTQSPGTLSLSPGERATL | 718 |
| | CSVSGGSVGIPSYYWSWIRQP | | SCRASQYVSSTYVAWYQHKPG | |
| | PGKGLEWIGFMYYTGSSNYNP | | QSPRLLISGASSRATGIPDRF | |
| | SLKSRVTISLDMSNNQFSLKL | | SGSASGTDFTLTISRVEPEDE | |
| | SSVTAADTAVYYCARESATTT | | AVYYCQQYGSSPYNFGPGTKV | |
| | WYRVGLYGLDVWGHGTTVTVS | | EIK | |
| | S | | | |
| AB-000248 | QVTLKESGPALVKPTQTLTLT | 258 | QSALTQPRSVSGSPGQSVTIS | 719 |
| | CTFSGFSLTTSGMRVSWVRQP | | CTGTSSDVGAYNYVSWYQQFP | |
| | PGKALEWLARIDWDDDKFYRT | | GKVPKLIIYDVGARPSGVPDR | |
| | SLKTRLTISKGTSKNQVVLTM | | FSASKSGNTASLTISGLQTDD | |
| | TNMEPADTATYYCARTVDSQF | | EADYYCCSFAHNYNVVFGGGT | |
| | DPWGQGTLVTVSS | | KVTVLS | |
| AB-000249 | QVQLHESGPGLVKPSQTLSLT | 259 | QSALTQPASVSGSPGQSITIS | 720 |
| | CTVSGASISSGGYYWTWIRQH | | CTGTSSDVGGHDLVSWYQQHP | |
| | PGKGLEWIGYIYYRGSTYYNS | | GRAPKLIIFEVTKRPSRISNR | |
| | SLRSRVTISVDATDNQFSLEL | | FSGSKSGNTASLTISGLRAED | |
| | RSVTAADTAVYYCARGGITSG | | EADYFCCSYAGPFISGVVFGG | |
| | WYVVGNWFDPWGQGTLVTVSS | | GTKLTVL | |
| AB-000251 | QVQLVQSGAEVKKPGASVKVS | 260 | DIQLTQSPSFLSASIGDRVTI | 721 |
| | CKASGYTFTAYNMYWVRQAPG | | TCRASQDLSSFLAWYQQKPGK | |
| | QGLEWMGWIDPSSGGTNYAQK | | APKFLIHAASTLQSGVPSRFS | |
| | FQGRVTMTRDTSFNTVYMELS | | GRKSGTEFTLTISSLQPEDYA | |
| | SLRSDDTALYFCARVGQWYPY | | TYYCLQLNNYFTTFGGGTKVE | |
| | YMDVWGQGTTVIVSS | | IK | |
| AB-000252 | QVQLVQSGAEVKKPGSSVTVS | 261 | EVVLTQSPATLSLSPGESATL | 722 |
| | CKASGDTITRYPITWVRQAPG | | SCRASQSVGSYLAWYQQKPGQ | |
| | QGPEWMGGIMPVYGRTEYAQK | | APRLLIYAASKRATGIPARFS | |
| | FQGRVTISADKSTNTVSMTLR | | GSGSGTDFTLTVSRLEPEDFA | |
| | IEDTAVYFCARMEHDSGWYPT | | VYYCQQRSSWPPYTFGQGTKL | |
| | GEYFRDWGQGTRVTVSS | | EIK | |
| AB-000253 | EVKLLESGGGLVQPGGSLRLS | 262 | QSALTQPPSASGSPGQSVTIS | 723 |
| | CGASGFTFSYYAMSWVRQAPG | | CTGPSSAVANYNYVSWYQQLP | |
| | KGLEWVSYISPSGDATQYADS | | GKAPKLILYEVNQRPSGVPDR | |
| | VKGRFTISRDNSKNTLYVQMN | | FSGAKSGNTASLTVSGLQAED | |
| | SLRVEDTAAYYCARVQSKGSS | | EGDYYCSSYGGTNNWVFGGGT | |
| | GWYWSGYYFDSWGPGTLVTVS | | KLTVL | |
| | S | | | |
| AB-000254 | EVQLLESGGGLVQSGGSLRLS | 263 | QSALTQPRSVSGSPRQSVTLS | 724 |
| | CAASGFTESSYWMHWVRQASG | | CTGTSSDVGGYNYVSWYQQHP | |
| | KGLVWVSRISTDGSSTTYADS | | GKAPKLMIYDVSKRPSGVPAR | |
| | VKGRFTISRDNAQNTLYLQMN | | FSGSKSGNTASLTISGLQAED | |
| | SLRAEDTAVYYCARGDSYYFG | | EADYYCCSYSGSYTPVVFGGG | |
| | SGSVRDWGQGTLVTVSS | | TKLTVL | |
| AB-000255 | QVQLQESGPRMVKPSETLSLT | 264 | EIVLTQSPATLSLSPGARATL | 725 |
| | CTVSGDSISSYNYYWTWIRQS | | SCRASQSISNYLAWYQQKPGQ | |
| | PGKGLEWIGSLHYGGSTYHNP | | APRLLIYDTYTRATGIPARFS | |
| | SLKSRVMISLDMSKNQLSLNL | | GSGSGTDFTLTISSLEPEDFA | |
| | ISVTAADAAVYYCARVWGDFG | | VYYCQQRSSWPLLTFGGGTRV | |
| | YGRFGIDWGQGALVAVSS | | EIK | |
| AB-000256 | LLQLQESGPGRVKPSETLSLT | 265 | EIVMTQSPATLSVSPGERATL | 726 |
| | CTVSGGSITTKSYYWSWIRQP | | SCRASQSIGSYLAWYQQKPGQ | |
| | PGKGLEWIGSIYSSGGTHYNP | | APRLLIFGASTRATGIPARES | |
| | SLKSRITTSVDASRNQVSLQL | | GSGSGTEFTLTISSLQSEEFA | |
| | KFVTAADTAIYYCARQTISGN | | VYYCQQYSNGWTFGPGTKVEV | |
| | RLIIAYFDTWGQGTQVIVSS | | K | |
| AB-000257 | QEQLVQSGAEVKKPGDSVKVS | 266 | QSVLTQPPSASGTPGQRVTIS | 727 |
| | CQASGYTFTGYYIHWVRQSPG | | CSGSSSNIGSNHVYWYQQVPG | |
| | QGLEWMGRINPNTGGTNYAQR | | TAPKPLIYLNSQRPSGVPDRF | |
| | FQGRVTMTRDTSISTVYMELS | | SGSKSGTSASLAISGLRSEDE | |
| | RLTSADTAVYYCARGRGYSYG | | ADYYCAAWDDSLSGYVFGSGT | |
| | DVFDIWGQGTMVTVSS | | KLTVL | |
| AB-000258 | QTQLVQSGAEVKKTGSSLKVS | 267 | QSALTQPPSASGSPGQSVTIS | 728 |
| | CKASGYSISYRYLHWVRQAPG | | CTGTSSDIGAYNFVSWYQQHT | |
| | QALEWMGCITPENGDTTSAQK | | GKAPKLIVYEVIKRPSGVPDR | |
| | FQDRVTITWDRSLSTVYMDLS | | FSGSKSGNTASLTVSGLQTED | |
| | SLRPEDTAMYYCASAPLKETS | | EADYYCSSYAGNKNVMFGGGT | |
| | DSWYRGTYYYYYYGLEVWGQG | | RLTVL | |
| | TPVSVSS | | | |
| AB-000259 | QMLLVESGGDLVKPGGSLRLS | 268 | DIQMTQSPSTLSASVGDRVTI | 729 |
| | CAASGFTFTDYFMTWIRQAPG | | TCRASQSISSWLAWYQQKPGK | |
| | KGLEWVAYVSSSSLYINYAYS | | APKLLIYKASSLESGVPSRFS | |
| | VTGRFTISRDNAKNLVFLQMT | | GSGSGTEFTLTISSLQPDDFA | |
| | SLRAEDSAIYYCARGHFDQFH | | TYYCQQYSSLWTFGQGTKVEI | |
| | FDYWGQGTLVTVSS | | K | |
| AB-000260 | QMSLVESGGGLVRPGGSLRLS | 269 | QPVMTQPPSASASPGASVTLT | 730 |
| | CAASGFTFTDYLMAWIRQAPG | | CTLGSDYSNYKVDWYQQRPGT | |
| | KGLEWVAYVSSSSLYVNYGES | | GPRFLMRVGPRGIVGSKGDGI | |
| | VTGRFTISRDNAKNSLFLQMT | | PNRFSVLASGLNRFLTIENLQ | |
| | SLRAEDSGIYYCAGGRFDQFH | | EEDEAVYHCAADQGNGDTLRW | |
| | FDYWGQGTPVSVSS | | VFGGGTKLTVL | |
| AB-000262 | QITLKESGPTLVKPTQTLTLT | 270 | QAVLTQPSSLSASPGASASLT | 731 |
| | CTFSGFSLSTPNMGVGWIRQP | | CTLRSGINVGIYRIYWYQQKP | |
| | PGKALEWLAVIYWNGDLRYSP | | GSPPQYLLRYKSDSDKQQGSG | |
| | SLRSRLTLTKATSESQVVLAM | | VPSRFSGSKDASANAGILLIS | |
| | TNMAPADTGTYYCARSIVTSD | | GLQSEDEADYYCMVWHSNAVV | |
| | NWFDPWGQGALVTVSS | | FGGGTKLTVL | |
| AB-000263 | EVQVVESGGGLVQPGGSLRLS | 271 | QAVLTQPSSLSASPGASASLT | 732 |
| | CAASGISVSDNYMTWVRQAPG | | CTLHSGIDVGLYRVYWYQQKP | |
| | KGLEWVSLISRGGDTFYADSV | | GSPPHYLLRYKTDSNKQQGSG | |
| | KGRFTISRDSFKNTLYLQMNS | | VSSRFSGSKDISANAGFLLIS | |
| | LRADDTAIYFCATDGSAHSYQ | | GVQSDDEADYYCMIWHNSAYV | |
| | DRDQDDYWGQGTLVTVSS | | FGGGTKLTVL | |
| AB-000264 | DVQLVESGGGLVQPGGSLKLS | 272 | DIQMTQSPSTLSASVGDRVTI | 733 |
| | CEASGFIFSAYPFHWVRQVSG | | TCRASQTIGTWLAWYQQKPGK | |
| | KGLEWLGRIRTKPNNFAPVYA | | APKLLIYEASSLQSGVPSRFS | |
| | ASVKGRFTISRDDSKKTAFLQ | | GSGSGTQFTLTISSLQPDDFA | |
| | MNSLKTEDTAIYYCTRSYYDT | | TYYCQQYNGYFLSFGPGTTVD | |
| | IGRYYFDYWGRGTLVTVSS | | IR | |
| AB-000265 | QVQVVESGGGVVQPGKSLRLS | 273 | QAGLTQPPSVSKGLRQTATLT | 734 |
| | CAASGFTFSHYAMHWVRQAPG | | CTGNSNNVGNQGATWLQHRQG | |
| | KGLEWVAVLSYDGSNKYYADS | | HPPKLLSYRNNNRPSGISEKL | |
| | VKGRFTISRDSSKNTLYLQMN | | SASRSGSTASLTITGLQPEDE | |
| | SLRAEDTAVYFCARDNFDYWG | | ADYYCSAWDSSLSAWVFGGGT | |
| | QGTLVTVSS | | KLTVL | |
| AB-000267 | EVQLVESGGGLVQPGESLRLS | 274 | QARLTQPPSVSEGLRQTATLT | 735 |
| | CVASGFTFTNYWMHWVRQPPG | | CTGNSNNVGHQGVSWVQQHQG | |
| | KGLEWVSRIDDGSGTSYATSV | | HPPKVLSYRTNNRPSGISERF | |
| | QGRFTISRDNSRNTVYLQMNS | | SASRSGNTASLTINGLQPEDE | |
| | LRAEDTAVYYCTTVFEFWAQG | | ADYYCSAWDKSLNAAVFGGGT | |
| | APVTVSS | | RLTVL | |
| AB-000268 | EVQLVESGGGLVKPGGSLRLS | 275 | QAGLTQPPSVSKDLGQTATLT | 736 |
| | CAASGFSFNYAWMNWVRQSPG | | CTGNSNNVGEQGAAWLQQHQG | |
| | KGLEWVGRIKSKTDGGTIDYA | | HPPKVLSYRNNNRPSGISERF | |
| | ASVKGRFTISRDDSENTLYLQ | | SASRSANTASLTITGLQPEDE | |
| | VNSLNTEDTAVYYCTTDLTYT | | ADYYCSAWDTSLSARVFGGGT | |
| | GSGGIDNWGQGTLVTVSS | | KLTVLS | |
| AB-000269 | EVQLVESGGGLVQPGGSLRLS | 276 | DIVMTQSPDSLAVSLGERATI | 737 |
| | CAASGFTFSDSVMHWVRQASE | | NCKSSQRIFYRSNNKNYLAWY | |
| | RRLEWVGHIETKANTYATAYA | | QQKPGQPPKLLIYWASTRESG | |
| | ASVKGRFTISRDDSKNTAYLQ | | VPDRFSGSGSGTDFTLTISSL | |
| | MNSLKTEDTAVYYCTRHASYS | | QAEDVAVYFCQQYYSTPPTFG | |
| | GSQSGDYWGQGTLVTVSS | | QGTKVEIK | |
| AB-000270 | QVHLVQSGAEVKKPGASVIVS | 277 | QAGLTQPPSVSKGLRQTATLT | 738 |
| | CTTSGYTFTSYGISWVRQAPG | | CTGNNNNVGNRGAAWLQQRPG | |
| | QGLEWMGWISAYRGTTNYAQK | | HPPKLLSYRNNNRPSGISERE | |
| | FQGRVTLTKDTSASTAYMELR | | SASRSGNTASLTITGLQPEDE | |
| | SLTSDDTAIYYCARDVDHDLD | | ADYYCSAWDNSLSARVFGGGT | |
| | NWGQGTLVTVSS | | KLTVL | |
| AB-000271 | EVQLVESGGGLVQPGESLKIS | 278 | EIVLTQSPATLSLSPGERATL | 739 |
| | CAASGFSFSDSAVNWVRQAPG | | SCRASQSVHSYLAWFQQKPGQ | |
| | KGLEWVGRIRNKARNDATIYA | | APRLLIYDASNRATGIPARFS | |
| | ASVEGRFTISRDDPKNTAYLQ | | GSGSGTDFTLTISSLEPDDFA | |
| | MNSLTIEDTATYYCTSRGPKD | | IYYCQQRYGWPPITFGQGTRL | |
| | TNMDDVILDYWGQGTLVTVSS | | EIK | |
| AB-000272 | QLQLQESGPGLVKPSETLSLT | 279 | QAVLTQPSSLSASPGASASLT | 740 |
| | CTVSDDSVSSSSYYWGWIRQP | | CTLRSGFNVGSYKIYWYQQKP | |
| | PGKGLEWIGNFYYGGNPYFNP | | GSPPQFLLKYKSDSDKQQGSG | |
| | SLKSRLSMSGDTSKNQFSLRL | | VPSRFSGSKDASANAGILLIS | |
| | SSVTAADTAVYYCARFSNWVF | | GLQSEDEADYYCMIWHSSAVV | |
| | DYWGQGTLVTVSS | | FGGGTKLTVL | |
| AB-000274 | QVQLVQSGAELKKPGASVKVS | 280 | QSVLTQPPSVSGAPGRRVTIS | 741 |
| | CRASGYTFTSYALHWVRQAPG | | CAGSSSNIGALYEVHWYQQLP | |
| | QRLEWMAWINAGNGITKYSQK | | GRAPKLLIYDNTNRPSGVPDR | |
| | FQDRVTITRDTSASIGYMELS | | FSGSKSGTSASLAISGLQAED | |
| | SLRSEDTAVYYCARPGIGIGL | | EGDYYCQSYDSRLSSPVFGGG | |
| | FNALDIWGQGTPVTVSS | | TKLTVL | |
| AB-000275 | EMQVVESGGGLVQPGGSWRLS | 281 | QTVVTQEPSFSVSPGGTVTVT | 742 |
| | CAASGFSVSNNYMMWVRQAPG | | CGLTSGSVSTINYPSWYRQTP | |
| | KGLEWVSIIHGNGRPDYAESV | | GQPPRTLIYNTNSRSSGVPGR | |
| | KGRFTISRDNSKNTLHLQMNS | | FSGSILGNKAALTITGAQADD | |
| | LRPEDTALYFCARYCTDISCP | | ESDYYCLLYLGPYTWMFGGGT | |
| | DMVITTAIAICGQGTMVTVSS | | RVTVL | |
| AB-000278 | EVHLVESGGDLVQPGGSLRLS | 282 | QTVVTQEPSFSVSPGGTVTLT | 743 |
| | CEASGFTVSSYYMSWVRQAPG | | CGLNSDSVSTRHYPSWYQQTP | |
| | KGPEWVSSIYKGGGTFYADSV | | GQAPRTLIYTTNIRSSGVPDR | |
| | KGRFAISRDIGKNTVFLHMNI | | FSASILGNKAALTITGAQADD | |
| | LRAEDTAVYFCASTLDSGNTY | | ESTYYCVLYMGRGIWLFGGGT | |
| | YFDSWGQGTLVTVSS | | RLTVL | |
| AB-000279 | QVHLVQSGAEVKKPGASVKIS | 283 | QSVLTQPPSVSGAPGQRVTIS | 744 |
| | CKASGYTFTTYSIHWLRQAPG | | CTGSSSNIGANYDVHWYQQLP | |
| | QSLEWMAWINAGNGNTKYSQK | | GTAPKLLIYRDNYRPSGVPDR | |
| | FQGRVTITRDTSATTAYMELS | | FSGSKSVTSASLAITGLQAED | |
| | NLRFEDTALYYCAKDPGYNYG | | EGDYYCQSYDDSLGGAIFGGG | |
| | GRWFDPWGQGTLVTVSS | | TQLTVLS | |
| AB-000280 | QVLLVESGGGVVQPGRSLRLS | 284 | SFELTQPSSVSVSPGQTARIT | 745 |
| | CAASGFTFRSYGMHWVRQAPG | | CSGDVLAKSFARWLQQKPGQA | |
| | KGLEWVAVIAFDGSEKYYADS | | PILVIYKDTERPSGIPERFSG | |
| | VKGRITISRDNSKNTVYLQMN | | SSSGTTVTLTISGAQVEDEAD | |
| | SLRAEDTAVYYCAKLDMDVWG | | YYCYSAADNVQGVFGGGTKLT | |
| | QGTTVTVSS | | VL | |
| AB-000281 | QVHLVESGGGLVKPGGSLSLS | 285 | QAVLTQPASLSASPGASASLT | 746 |
| | CAASGITVSDYHMTWIRQAPG | | CILRSGINVGTYMIYWYQQKP | |
| | KGLEWVAYSSSSGSNTNYADS | | GSPPQFLLRYKSDSDKKQGSG | |
| | VKGRFTISRDNAKNSLYLQMN | | VPSRFSGSKDASANAGILLIS | |
| | SLRNEDTAVYYCARLFFVYYD | | GVQSEDEADYYCMIYHSSAWV | |
| | FPRENGMDVWGQGTTVTVSS | | FGGGTKLTVL | |
| AB-000282 | QLQLQESGPGLVKPSETLSLT | 286 | QSVLTQPPSVSGAPGQRVTIS | 747 |
| | CSVSGGSISGTSHFWAWIRQP | | CTGSSSNIGANYDVHWYQQIP | |
| | PGKGLEWIGSIYWSGATYYNP | | GTAPKLLIYGNINRPSGVPDR | |
| | SLKSRVTMDVDTSKKQFSLKL | | FSGSKSGTSASLAITGLQPED | |
| | SSVTAADTAVYYCASQSYDIQ | | EADYYCQSFDSSLSGQHAVFG | |
| | TGWAAFFYYYHIAVWGKGTTV | | GGTHLTVL | |
| | TVSS | | | |
| AB-000283 | QVKLEESGGGVVQPGRTLRLS | 287 | AIQMTQSPSSLSASVGDRVTI | 748 |
| | CVVSGFTFSNYGMHWVRQAPG | | TCRASQGINVFLNWYQQKPGK | |
| | QGLEWVALIWKDGTNKYYAES | | APKLLIHAASILRTGVPSRFS | |
| | VQGRCTISRDNSKNTLYLRLN | | GSGSGTYFTLTIRGLQPEDFA | |
| | TVRDEDTAIYYCAREDREVVP | | TYYCQQSYGTPPAITFGGGTR | |
| | AAFDYWGQGALVTVSS | | IEIK | |
| AB-000284 | QEHLVEAGGGVVQPGKSLRLS | 288 | SSELTQPSSVSVSPGQTARIT | 749 |
| | CAASGFSFSRYGIHWVRQAPG | | CSGDILAKNYARWFQQKPGQA | |
| | KGLEWVAVISYDGTNRYYADS | | PVLVIYKDTERPSGIPERFSG | |
| | VKGRFTISRDNSKKTVYLQIN | | STSGATVTLTISGAQVEDEAD | |
| | SLRVEDTAVYFCSKLEMDYWG | | YYCYSATDNGLGVFGGGTKLT | |
| | RGIQVTVSS | | VL | |
| AB-000286 | EVQLAESGGGLVKPGGSLRLS | 289 | QTVVTQEPSFSVSPGGTVIIT | 750 |
| | CAASGFALSRYSMNWVRQAPG | | CGLRSGSVSASHYPSWYQQTP | |
| | KGLEWVSSISSSSNYIFYGDS | | GQPPRTLIYTTSTRSSGVPDR | |
| | VKGRFTISRDNAKNSLYLQMN | | FSGSILGNKAALTITGAQSDD | |
| | GLRAEDTAVYYCARWVTAATS | | ECDYYCVLYMGNGISVFGGGT | |
| | YYYYMDVWGKGTTVTVSS | | KLTVL | |
| AB-000287 | QVQLVQSGAEVMKPGASVKVS | 290 | QSVLTQPPSVSGAPGQRITIS | 751 |
| | CKASGYTLNNYDVNWVRQASG | | CTGTTSNIGAGYDVHWYQQLP | |
| | QGPEWMGWMRPGGDTGFAHKF | | GAAPRLLIHGTSSRPSGVPDR | |
| | QGRVSITRDTSINTAYMELSS | | FSGSYSGSGSDSGASRYLAIT | |
| | LRSEDTAVYFCARGSAGGQSY | | GLRAEDEADYYCQAFDNIVGG | |
| | FNGLDVWGQGTPVIVAS | | WVFGGGTKVTVL | |
| AB-000288 | QITLKESGPTLVKPTETLTLT | 291 | QSVLTQPPSVSGAPGQRVTIS | 752 |
| | CTFSGSSVSTSGLGVGWIRQA | | CTGSSSNIGAGYDVHWYQQLP | |
| | PGKALEWLAISYWNHDQRYNP | | RVAPKLLIYGNNNRPSGVPDR | |
| | SLMSRLTTTKDDSRDQVVLTM | | FSGSKSGTSASLAITGLQAED | |
| | TNMDPMDTGTYFCAHRRLSSL | | EADYYCQSYDSSLSGSFVFGT | |
| | KSAGYYDHWGPGILVTVSS | | GTKVTVL | |
| AB-000289 | QVQLVQSGSELKKPGASVKVS | 292 | KIVLTQSPATLSLSPGERATL | 753 |
| | CKASGYSFTNYAIDWVRQAPG | | SCRASQNVRTYIAWYQHKVGQ | |
| | QGLEWMGWINTNTGNPTSAPG | | APRLLVYDASNRATGIPGRES | |
| | FTGRFVFSLDTSVRTAYLQIS | | GSGSGTDFTLTISSLEPEDFA | |
| | NLKAEDTAIYYCARESLVAAG | | IYYCQHRGNWLTFGGGTKVEI | |
| | GHYQYNGMDVWGQGTTVTVSS | | K | |
| AB-000291 | QVQLVESGGGVVQPGRSLRLS | 293 | QPVLTQTPSVSGPPGQRVTIS | 754 |
| | CEASGFSFSNYGLQWVRQPPG | | CSGSSSNMGNNNVYWYQQFPG | |
| | KGLEWVAFILNDGRTKNYVDS | | MAPKLLIYRSSQRPSGVPDRF | |
| | VKGRFTISRDNSKNTLYLEMD | | SGSKSGTSASLAISGLQSEDE | |
| | SLRAEDTAVYYCARHKFGDGY | | AIYHCASWDDSINGWVFGGGT | |
| | GMDVWGQGTTVIVSS | | ELTVL | |
| AB-000293 | DVQLVESGGGLVQPGGSLRLS | 294 | DIVMTQTPLSSAVTLGQPASF | 755 |
| | CTASGFTFSTYWMTWVRQIPG | | SCMSSQSLVHRDGTTYLTWLQ | |
| | KGLEWVATIERRGDKKHYVDS | | QRPGQPPRLLIYKISNRFSGV | |
| | VKGRFTISRDNAQSSLYLQMD | | PDRFTGSGAGTDFTLKISRVE | |
| | SLRVDDTAMYYCVRENWVEDY | | AEDVGVYYCMQTTQFPYTFGQ | |
| | WGQGVLVTVSS | | GTKLEIK | |
| AB-000294 | QVQVVESGGGVVQPGRSLTLS | 295 | AIQLTQSPSSLSASIGDRVTI | 756 |
| | CAGSGFTENSYAIHWVRQAPG | | TCRTSQGISSTLAWYQQRPGK | |
| | KGLEWVAVSVSSDGHYKYYAD | | PPKLLIYDASTLDDGVPSRFS | |
| | SVQGRFTISTDSSTSTLYLHM | | GSRFGTDFTLTISSLQPEDFA | |
| | NSLRVEDTALYYCARAWGLSR | | TYYCQQFNNYPLTFGGGTKVE | |
| | GVIIGYWGQGTLVTVSS | | IR | |
| AB-000295 | QDQLEQSGAELKKPGASMKVS | 296 | QSVLTQPPSVSGAPGQRVIIS | 757 |
| | CKFSGDSFTRFAIHWLRQAPG | | CTGSNSNIGANYDVHWYQQVP | |
| | QGLEWMGWINTGNGLTKYAPR | | GRAPKVLIYGDTNRPSGVPDR | |
| | FQGRLSITRDTSANTAYMELT | | FSGSKSGASASLAITGLQAED | |
| | SLTSTDTAVYYCARATDCSGP | | EADYYCQSYDRRLSGSIIFGG | |
| | RCFVLGWYDPWGQGTLVTVSS | | GTKVTVL | |
| AB-000297 | QVQLVESGGGVVQPGRSLRLS | 297 | SYELTQPSSVSVSPGQTARIT | 758 |
| | CAASGFTFSRYGMHWVRHAPG | | CSGNLLAKNFARWFQKKPGQA | |
| | KGLEWVAAISYDGRNKYYGDS | | PVQVLYKDSERPSGIPGRESG | |
| | MKGRFTLSRDNPKNMVYLQMN | | SSSGTTVTLTIRGAQVDDEAD | |
| | SLKPDDTGVYYCAGDLAHWGQ | | YYCYSATDNSLGVFGGGTKLT | |
| | GTLVTVSS | | VL | |
| AB-000298 | EVQVVESGGGLVQPGLSLRLS | 298 | DIQMTQSPSSLSASVGDRVTI | 759 |
| | CTASGFPFGAYAMSWVRQAPG | | TCRASQGIRNDLGWYQQKPGQ | |
| | KGLEWVSLIRIKTSGGTKHYA | | PPERLIYAASNLQSGVPSRFS | |
| | ASVEGRFTISRDDSRGILYLH | | GSGSGTEFTLTISSLQPEDFA | |
| | MDSLKTEDTAVYYCARGFTNP | | TYYCLQHNSYPWTFGQGTKVE | |
| | FDYWGQGTPVTVSS | | IK | |
| AB-000299 | DVQLVESGGGLVQPGGSLRLS | 299 | DIVMTQTPLSSPVTLGQPASI | 760 |
| | CAVSGFTFSDHYIDWVRQAPG | | SCRASQSLVHSKGDTFLSWFH | |
| | KGPEWVGRNRNKANNYSTEYA | | QRPGQPPRLLIYKVSNRLSGV | |
| | ASVRGRFTVSREESTTSVHLQ | | PDRFSGSGAGTDFTLKISRVE | |
| | MSSLRTDDTAMYYCVRVYAEP | | AEDVGVYYCMQNTQYPITFGQ | |
| | GKYYFDFWGQGTQVTVSS | | GTRLEIK | |
| AB-000301 | QLQLQESGPGLVKPSETLSLT | 300 | QSVLTQPPSVSGAPGQRVTIS | 761 |
| | CTVSGGSISRSSNYWGWIRQP | | CTGSSSNIGAGYDVHWYQQLP | |
| | PGKGLEWLGSIYFSGTTNYNP | | GTAPKLLIYVNNNRPSGVPDR | |
| | SLKSRVTISVDTSKNQFSLNL | | FSGSKSGTSASLAITGLQAED | |
| | NSVTAADTAVYYCASQSYSRS | | EADYYCQSYDSSLSGSVVFGG | |
| | SGWALIYYYYFMDVWGKGTTV | | GTKLTVL | |
| | TVSS | | | |
| AB-000302 | QVQLQQSGPGLVKPSQTLSLT | 301 | DIVMTQSPDSLAVSLGERATI | 762 |
| | CVVSGDSLFSNGVAWNWFRQS | | SCKSSQSVFHTSYNRHFIAWY | |
| | PSRGLEWLGRTYYRSKWYNEY | | QQKAGQPPKLLIYWSSTRESG | |
| | EVSVKSRIMIDPDTSKIQFSL | | VPDRFSGSGSGTDFTLTISSL | |
| | QLNSVTPEDTAVYYCARGRNN | | QAEDVAFYYCQQYHSAPPTFA | |
| | AFDFWGQGTTVTVSS | | QGTKVEIK | |
| AB-000303 | EVQVLESGGGLVKPGGSLRLS | 302 | SYELTQPSSVSVSPGQTARIT | 763 |
| | CATSGFPFSNAWMNWVRQAPG | | CSGDVLAQKYVRWFQQKPGQA | |
| | KGLEWVGRIKSKTDGGTPDYA | | PVLVIYKDTERPSGIPERFSG | |
| | APVKGRFTISRDDSKNRLYLQ | | SISETTVTLTISGAQVEDEAD | |
| | MNSLKTEDTAVYYCTTAFDYW | | YYCYSATDKWGVFGGGTKLTV | |
| | GQGTLVTVSS | | L | |
| AB-000304 | QVQLVESGGGVVQPGRSLRLS | 303 | SYELTQPSSVSVSPGQTARIT | 764 |
| | CVGSRFTFSRYGMHWVRQAPG | | CSGDILAKSYARWFQKKPGQA | |
| | KGLEWVAVVSYDGSNKYYVDS | | PVLVIYKDTDRPSGIPERFSG | |
| | VKGRFTISRDNSKNTLYLQMN | | SSSGTTVTLTISGAQVEDEAD | |
| | NLRAEDTAVYYCAKDLDVWGK | | YYCYSAADNVQGFFGGGTKVT | |
| | GTTVTVSS | | VL | |
| AB-000306 | QVQLQQPGPRLVKPSQTLSLT | 304 | DIVMTQSPDSLAVSLGERATI | 765 |
| | CAISGESVSSNTAAWNWIRQS | | HCKSSQSVLYNSNNKHYIAWT | |
| | PSRGLEWLGRTYYRSKWITDY | | QQKPGQPPKLLIYWASTRESG | |
| | AVSLRSRMTINSDTSNNQVSL | | VPDRFSGSGSGTEFALTINNL | |
| | HLTSVTLEDTAVYYCAYGLDV | | QAEDVAVYYCQQYYTIPITFG | |
| | WGQGTTVIVSS | | QGTRLEIK | |
| AB-000309 | EVQLFESGGGLVQPGGSLRLS | 305 | EIVLTQSPGTLSLSPGERATL | 766 |
| | CAASGFTFSTCGMSWVRQSPT | | SCRASQSVTSDFLAWYQQKPD | |
| | KGLEWVSSISASGDLTFYADS | | QAPRLLIYSASSRATGIADRF | |
| | VKGRFTISRDNSKNTLFLQIN | | SGSGSGTDFTLTISGLEPEDF | |
| | NLRADDTAIYYCAKIGVIARW | | ALYYCEQYGSTPPTFGQGTKV | |
| | YYDFWGRGNLVTVSP | | EIK | |
| AB-000313 | QVQLVESGGGVVQPGRSLRLS | 306 | DIQMTQSPSTLSASVGDRVTI | 767 |
| | CAASGFTFSSYGMHWVRQAPG | | TCRASQSISSWLAWYQQKPGK | |
| | KGLEWVALIWYDGSNKYYADS | | APKLLIYQASSLESGVPSRFS | |
| | VKGRFTISRDNSKNTLYLQMN | | GSGSGTEFTLTISSLQPDDFA | |
| | SLRAEDTAVYYCARVRDTSGY | | TYYCQQYNSYWTFGQGTKVEI | |
| | HGDAFDIWGQGTMVTVSS | | K | |
| AB-000316 | QEQLVESGGGVVQPGRSLRLS | 307 | DIQMTQSPSTLSASVGDRVTI | 768 |
| | CAASAFTFSSHGMHWVRQAPG | | TCRASQNINSWLAWYQQKPGK | |
| | KGLEWVAIIWFDGSQKYYRDS | | APKLLIYKASSLESGVPSRFS | |
| | VKGRFTISRDNSKNTLFLQMN | | GSGSGPDFTLTISSLQPDDFA | |
| | SLRAEDTAVYYCARTRQGEDY | | TYYCHQYYRYWTFGQGTKVEI | |
| | DSLIGDAFDLWGQGTMVTVSS | | K | |
| AB-000318 | EVQLLESGGGLVQPGGSLRLS | 308 | SSELTQDPAVSVALGQTVRIT | 769 |
| | CAASGFTFNNYAMTWVRQAPG | | CQGDSLRRYDASWYQKKPGQA | |
| | KGLEWVSIISASGDSTNYADS | | PVFVMYGKNNRPSGIPDRFSG | |
| | VKGRFTISRDNSKKKLYLQMN | | SSSGNTASLTITGAQAEDEAD | |
| | SLRAEDTAVYYCAKVVPEYNN | | YYCNSRDSSGYQVVFGGGTKV | |
| | GWYSYYYGYMDVWGTGTTVSV | | TVI | |
| | SST | | | |
| AB-000319 | QVLLQQWGAGLLKPSETLSLT | 309 | EIVLTQSPGTLSLSPGERATL | 770 |
| | CADYGGSFSYYYWTWIRQPPG | | SCRASQSVSSSFLAWYQQKPG | |
| | KGLEWIGEINHSGSTNYSPSL | | QAPRLLIFDASSRATGIPDRF | |
| | KSRVTISVDASKSRESLRLTS | | SGSGSGTDESLTISRLEPEDF | |
| | VTAADTAVYYCARGRPPYISG | | AVYFCQQYDSSPWTFGQGTKV | |
| | WRIPGKYFFDYWGQGALVTVS | | EIK | |
| | S | | | |
| AB-000321 | EVQLLESGGGLVQPGGSLRLS | 310 | AVVMTQSPLSLSVTLGQPASI | 771 |
| | CEASGFTFSATGMTWVRQAPG | | SCRSTQSLVCADGHVCLNWFH | |
| | KGLECVSAIGVNVANTDYADS | | QRPGQSPRRLIYHVSNRDSGV | |
| | VKGRFTIFRDNSKNTLYLQMS | | PDRVSGSGSDTDFTLKISRVE | |
| | SLRAEDTAVYYCAKGTTNIDY | | AEDVGVYYCLQGTHWPRTFGQ | |
| | WGQGIQVTVSS | | GTKVEIK | |
| AB-000322 | EVQLVESGGGLVQPGGSLRLS | 311 | QTVVTQEPSLTVSPGGTVTLT | 772 |
| | CAASGFSFSSHYMAWVRQAPG | | CASSTGAVTSTYYPNWFQQKP | |
| | KGLEWVGNIRQDASAAFYVDS | | GQAPRALIYGINNKHSWTPAR | |
| | LKGRFTISRDNAKNLLFLQLN | | FSGSLLGGKAALTLSGVQPED | |
| | SLRAEDTAVYYCARWTYSSVW | | EAEYYCLLYSGSGWVFGGGTK | |
| | FLDYWGQGTLVTVSS | | VTVL | |
| AB-000323 | QVQLVQSGAEVKKPGSSVKVS | 312 | DIVMTQSPDSLAVSLGERATI | 773 |
| | CEASGGGFSNFGISWVRQAPG | | TCKSSQSLLYSSNSQNYLAWF | |
| | QGLEWMGRIVPLVHITDYAQK | | QQKPGQPPRLLIFWAYFRESG | |
| | FQGRVTITADRSTSTVYMELR | | VPDRFSGSGSGTDFSLTIGSL | |
| | SLRSEDWAVYYCARSKYQLLY | | QAEDVAVYFCQQYLSFPWTFG | |
| | VARTDLLDNWGQGTLVKVSS | | QGTKVEIK | |
| AB-000324 | QVHLVESGGGVVQPGRSLRLA | 313 | SYVLTQPPSVSVAPGKTARIT | 774 |
| | CAASGFTFSSYGLHWVRRAPG | | CGGNNIRNKNVHWYQQKPGQA | |
| | KGLEWVALIWSDGSNHYYGDS | | PVLVVYDDDDRPSEIPERFSG | |
| | VKGRFTISRDNLKNTLYLQMN | | SNSGNTATLTISRVEAGDEAA | |
| | SLRAEDTAVYYCAREKRIAAP | | YYCQVWDTSSNHVVFGGGTEL | |
| | AAFDYWGQGTLVTVSS | | TVL | |
| AB-000326 | QVQLQESGPGLVKPSETLSLT | 314 | DIVMTQSPLSLPVTPGEPASI | 775 |
| | CTVAGGSISTYYWSWIRQSAG | | SCRSSQSLLHSDGSNYLDWYL | |
| | KGLEWVGRIKSNGNTHYNPSL | | QKPGQSPQLLIYLGSNRASGV | |
| | KSRVTMSIDSSKNQVSLKMSS | | PDRFSGSGSGTDFTLKISRVE | |
| | VIAADTAVYYCARLNLPASRG | | AEDVGVYYCMQTLLVPYTFGQ | |
| | AFDIWGQGTMVTVSS | | GTKLEIK | |
| AB-000328 | EVQLQQSGGEVKKPGESLRIS | 315 | AIVMTQSPDSLALSLGERATI | 776 |
| | CQGSGYTFSNYWIGWVRQMPG | | NCKSSQSVLYSPNKKSYLAWY | |
| | KGLDLVGLIYPRDSDTRYSPS | | QQKPRQPPKLLIYWASTRKSG | |
| | FQGRVTISADKSVSTAFLQWS | | VPDRFSGSGSGTDFTLTISSL | |
| | SLQASDSAMYFCARGLGGGTY | | QAEDVAVYYCQQYFDIPTFGQ | |
| | GGDFDSWGQGTLVTVSS | | GTRLEIK | |
| AB-000329 | QVQLQESGPGLVKPSQTVSLT | 316 | EVVLTQSPGTLSLSPGERATL | 777 |
| | CTVSGGSISSRSNYWSWIRQP | | FCRASQSVTTSLGWYQQRPGQ | |
| | AGKGLEWIGHIYTSGNTNYNP | | PPRLLIYEASNRATGIPARFS | |
| | SLKSRVTISRDTSKNQFSLKL | | GSGSGTDFTLTISSLQPEDFA | |
| | SSVTASDTAVYYCARDLGLSS | | VYYCQHRNNWPPRYTFGQGTK | |
| | NWDIHWYFDLWGRGTLVTVSS | | LEIK | |
| AB-000330 | QVQLVQSGAEVREPGSSVKVS | 317 | QAVVTQEPSVTVSPGGTVTLT | 778 |
| | CKAPGGIFSYYAFSWVRQAPG | | CASSAGAVTGGHYPGWFQQKP | |
| | QGLQWMGGLVPALGATTYAQR | | GQTPRSLIHTTGNKQSWTPAR | |
| | FRDRVTITADASSRTVYMELR | | FSGSLLGGKAALTLSAVQPED | |
| | NLRSEDTAIYYCARDQGELAT | | EADYYCLLYYGGSPVFGGGTK | |
| | IAPYYSYYTLDVWGQGTTVTV | | VTVL | |
| | SS | | | |
| AB-000331 | EVQLVESGGGLVQPGGSLRLS | 318 | QSVLTQPPSVSGTPGQRVIIS | 779 |
| | CVCSGFTFSNYWMTWVRQAPG | | CSGSSSNTGSTYVYWYQQLPG | |
| | KGLEWVANIKKDGSEKYYVDS | | TAPKLLIYRNDQRPSGVPDRF | |
| | VRGRFAISRDNAKTSLYLEMN | | SGSKSGTSASLAISGLRSEDE | |
| | SLRVEDTAVYYCRSLLDPAYN | | ADYHCAAWDDSLRGPVFGGGT | |
| | YGRGDGRWGQGTLVTVSS | | KLIVLS | |
| AB-000333 | QVQLVESGGGVVHPGRSLRLS | 319 | DVQVTQSPSSLSASVGDRVSI | 780 |
| | CAASGFSFSRYGMHWVRQAPG | | TCRASQTLSIAWYQQKPGKAP | |
| | KGLEWLALTWHDGSNKHYADS | | KLLIYAASRLQSGVPSRFSGS | |
| | AKGRFTIYRDNSKNMLFLQIN | | GSGTDFTLTISSLQLEDGATY | |
| | SLRVEDTAVYYCAREVPEVAM | | YCQQSNLTPLLTFGGGTKVEI | |
| | AVLDYWGQGTLVTVSS | | K | |
| AB-000336 | QVQLQESGPGLVKPSGTLSLT | 320 | VIQLTQSPPSLSASVGDRVTI | 781 |
| | CAVSGVSISGSHWWSWVRQSP | | TCRASQYISTYLNWYQQKPGK | |
| | GKGLEWIGEIFHAGLTNYNLS | | APKLLLYAASTLQSGVPSRFS | |
| | LKSRVTMSVDKSKNQFSLTLT | | GSGSGTDFTLTISSLQPEDFA | |
| | SVTAADTAVYYCARVMVWITA | | TYYCQQSYSAPLLSQSRYQFT | |
| | THEDSWGQGTLVSVSS | | FGPGTTVDIK | |
| AB-000339 | EVQLVESGGGLVQPGGSLRLS | 321 | QSVLTQPPSASGTPGQRVTIS | 782 |
| | CAASGFTLSDHYMDWVRQAPG | | CSGSSSNIGIRNVNWFQQLPG | |
| | KGLEWVGRIRTQVRSYDTDYA | | TAPKLLMYNNNQRPSGVPDRF | |
| | ASVKGRFTVSRDDSRNSVYLQ | | SGSKSGTSASLAINGLQSEDE | |
| | MNSLKTEDTAVYYCVRIGPIG | | ADYYCSAWDESLKGVIFGGGT | |
| | YDGMDVWGQGTTVTVSS | | KVTVL | |
| AB-000340 | QLQLQESGPGLVKPSETLSLT | 322 | QAVLTQPPSVSGAPGQRVTIS | 783 |
| | CSVSSASISSTSSYWAWIRQP | | CTGSSSNIGAGFDVHWYQQLP | |
| | PGKGLEWIGSIYYSGNTFYNP | | GTAPKLLVYGNNNRPSGVPDR | |
| | SLRSRLTISVDTSKKQFSLTL | | FSASKSGTSASLAIAGLQAED | |
| | RSVTAADTAVYYCARQSLYTR | | EADYYCQSYASTLRDVVFGGG | |
| | SWYIESYFDYWGQGTVVTVSS | | TKLTVL | |
| AB-000341 | QLQLQESGPGLVKPSETLSLT | 323 | SYELSQSLSVSVALGQTASIT | 784 |
| | CTVSGGSISSMSHYWGWIRQP | | CGGDNIGTKNVHWYQQRPGQA | |
| | PRKGLEWIGSIYYSGTTYYNP | | PVLVMDRDTNRPSGIPERFSG | |
| | SLKSRVTISVDTSKNQFSLRL | | SNSGNTATLSINRAQAGDEAD | |
| | SSATAADTAIYYCARQVAYRS | | YYCQVWDSNTMIFGGGTKLTV | |
| | GWYVYYYVDVWGKGTTVTVSS | | L | |
| AB-000342 | QVQLQESGPGLVKPSETLSLT | 324 | QSVLTQPPSASGTPGQRVTIS | 785 |
| | CSVSSGFISNYYWGWIRQPPG | | CSGSNSNIGSDTVNWYQQLPG | |
| | KGLEWIGYIHHNGRSDYSPSL | | TAPKLLIYRNNQRPSGVPDRF | |
| | KSRVSILLDTPKNQISLRLTS | | SASKSGTSASLAISGLQSEDE | |
| | VTAADTAMYYCAGDRYSAYNP | | ADYYCAAWDDSLRGPVFGGGT | |
| | SAFEIWGHGTKVTVSS | | KLTVLS | |
| AB-000343 | DVQLLESGGGLVQPGGSLRLS | 325 | QSALTQPASVSGSPGQSITIS | 786 |
| | CEASGFTFNIFGMSWVRQTPG | | CTGTSSDVGGYNYVSWYQQHP | |
| | KGLEWVSGISPSGDSTYYSDS | | GKAPKLMIYDVSNRPSGVSNR | |
| | VKGRFTISRDNSKHTVYVQMK | | FSGSKSGNTASLTISGLQAED | |
| | SLRAEDTAVYYCAKTSPQYIN | | EADYYCGSYTSTSTVVFGGGT | |
| | GWYAKWINYFDYWGRGTLVTV | | KLTVL | |
| | SS | | | |
| AB-000344 | QVQLVESGGGVVQPGRSLRLS | 326 | DIQLTQSPSSLSASVGDRVTI | 787 |
| | CEVSGFFFSRYGMHWVRQAPG | | TCRASRNIGDFLNWYHQKPGK | |
| | KGLEWVGCIWHDGSNQYYGDS | | APKLLIYAASHLQSGVPSRFS | |
| | VKGRFTISRDNSKNTFYLQMN | | GSGSGTYFTLTVSGLQPEDFG | |
| | RLRVDDTAVYYCAREGTTAVT | | TYYCQHSDTSPQITFGQGTRL | |
| | IYDYWGQGTLVTVSS | | EIK | |
| AB-000345 | EVQLVESGRNLVRPGGSLRLS | 327 | EIVMTQSPATLSVSPGERATL | 788 |
| | CAASGFGFTTHAMSWIRQAPG | | SCRASQSISRNLAWYQQKPGQ | |
| | KGLEWVSLITGNGETTAYIDS | | APRLLIYDASTGATGVPARFR | |
| | VKGRFTISRDNSKNMLYLQMN | | GSGSGTEFTLTISSLRSEDFA | |
| | SLRVEDTAIYYCVKGYGLFDF | | FYYCQQYYGWPPWTFGQGTKV | |
| | WGQGTLVTVSS | | EIK | |
| AB-000346 | QVQLVQSGAEVKKPGASVRIS | 328 | QSALTQPPSVSGSPGQSVTIS | 789 |
| | CKASGHTFNAFHIHWVRQAPG | | CTGSSSDVDIYDHVSWYQQPP | |
| | QGFEWMGKFFSSGDQTRFPQK | | GTVPKAVIFEVTNRPSGVPDR | |
| | FQGRITMTRDTSTSTAYMELT | | FSGSKSGNAASLTISGLQPED | |
| | SLTSDDTAVYYCAREGPFTYY | | EADYYCTSYTTRDGFFFGTGT | |
| | FDYWGQGTLLTVSS | | KVTVL | |
| AB-000347 | EVQLLESGGGLVQRGGSLRLS | 329 | AYVLTQPPSVSVAPGQTARIT | 790 |
| | CAASQFAFSKYTMTWVRQAPG | | CGGNNIGSYSVCWYQQKPGQA | |
| | KGLDWVAAISGSSEHTYYADS | | PVLVINDDSDRPSGIPERFSG | |
| | VKGRFTISRDNSKNTVFLQMS | | SNSGNTATLTITRVEVGDEAD | |
| | SLRADDTAVYYCAKDGVMVSA | | FYCHVWDRGSDQGVFGGGTKL | |
| | TPWGMDVWGRGTTVIVSS | | TVL | |
| AB-000348 | QLLLQESGPGLVKPSETMSLT | 330 | DIQMTQSPSFLPASVGDRVTI | 791 |
| | CSVSGDSLSSDENFWGWTRQS | | TCRATQSITNYVNWHRQRPGS | |
| | PGKGLEWIGSISPRGATYYNP | | APESLIYSTSLLHFGVPSRFS | |
| | SLQSRVTMSIDTSKNELSLKL | | GDGSGTRFTLTISSLRPEDFA | |
| | TSVTAADTAVYLCVKGHGNIS | | TYYCQQTLRVPFTFGGGTTIV | |
| | KWGQGTLITVSS | | AN | |
| AB-000351 | QVHLVQSGGEVKRPGASVKVS | 331 | DIQMTQSPSTLSASVGDRVTI | 792 |
| | CKASGYTFNNHGVSWVRQAPG | | TCRASQSITRWLAWYQQKPGK | |
| | QGLEWMGWVSTHKSNTNHAQR | | APKLLIYKASTLESGVPSRFS | |
| | FQGRVTMTTDTSTTTAYLEVR | | GSGAGTEFTLTISSLQPDDFA | |
| | SLRSDDTAVYYCATGRLFGVT | | TYYCQQYNTYSPPFTFGQGTK | |
| | IPDAFDIWGQGTLVTVSS | | LEIK | |
| AB-000353 | QAQLMQSGAEVKKPGASVKVS | 332 | DIQMTQSPSSLSASVGDRVTI | 793 |
| | CKASGYSFTSYGISWVRQAPG | | TCRASQSIATYVNWYQQRPGK | |
| | QGLEWMGWISTYNGNTDYAQT | | APRLLIYTASNLQSGVPSRFS | |
| | LQGRVTMTTDTSTSTAYMELR | | GSGSGTDFTLTISNLQPEDFA | |
| | SLRSDDTAVYYCVRVVAYCIG | | TYYCQQSYSTPPYTFGLGTKL | |
| | GSCYAVGYFDYWGQGTPVTVS | | EIK | |
| | S | | | |
| AB-000354 | QVQLVESGGGVVQPGRSLRLS | 333 | SYELTQPSSMSVSPGQTARIT | 794 |
| | CAASGFTFNSYGMHWVRLAPG | | CSGDILAKNYARWLQQKPGQA | |
| | KGLEWVAVISYDGTKKYYADS | | PVLLIHKDRERPSGIPERFSG | |
| | VKGRFSISRDNSKNTLDLQMN | | SSSGTTVTLTISGAQVEDEAD | |
| | SLRLEDTAVYYCAKLDFDYWG | | YYCYSATDNSLGVFGGGTKLT | |
| | QGTLVTVSS | | VLS | |
| AB-000357 | QVQLVQSAAEVKKPRSSVKVS | 334 | EILMSQSPAILSVSPGERVTL | 795 |
| | CKASGGTFGNNAISWVRQAPG | | TCRASESVSTNLAWYQQKPGQ | |
| | QGLEWMGGIVPFLTTVNYAQK | | APRLLIYGASTRATGIPARFS | |
| | FQDRVTITADRSTNTAYMELV | | GSGSGIEFTLTISSLQSEDLA | |
| | RLTSEDTAVYYCARVRRSGYN | | VYYCQQYNKWFALSFGGGTKV | |
| | SVYYFDMWGQGTLVTVSS | | EIK | |
| AB-000358 | QLHLQESGPGLVKPSETLSLT | 335 | EIVLTQSPGTLSLSPGERATL | 796 |
| | CTVSGGSIRSSSYYWGWLRQP | | SCRASQTLSSAYLAWYQQKPG | |
| | PGKDLEWIGSIYSSGTTYYSP | | QAPRLLIYGTSRRATGVPDRV | |
| | SLESRVTVLVDTSKNQFSLRL | | RGSGSGTDFTLTISRLEAEDS | |
| | TSVTAADTAVYYCARPVHLDY | | AVYYCQQYGGSPLTFGGGTKV | |
| | STISYYPGNGPFDIWGPGTLV | | EMK | |
| | TVSA | | | |
| AB-000359 | EVQLVGTGGGLVQPGRSLTLS | 336 | DIVMTQTPLSSPVTLGQPASI | 797 |
| | YAASGFNFGDYTMSWVRQAPG | | SCRSSQSLVHSDGNTYLNWLH | |
| | KGLEWLGLIREKPRGGAAEYA | | QRPGQPPRLLIYKVSNRFSGV | |
| | ASVRDRFTISRDDSKSIAYLQ | | PDRFSGSGAGTDFTLKISRVE | |
| | MSSLRTEDTAVYYCIPLAWSP | | AEDVGIYYCMQATHFTWTFGQ | |
| | RDGYNGIFDSWGQGTLVTVSE | | GTKVEIK | |
| | GVSA | | | |
| AB-000360 | EVQLVESGGGLVQPGRSLRLS | 337 | DIQMTQSPSSLSASVGDRVTI | 798 |
| | CTASGFTFGDYVMSWVRRAPG | | TCRASRAISTSLAWYQQKSGK | |
| | KGLEWLGFIRSKLYGGTPEYA | | GLRLLIQAASSLQSGVPSRFS | |
| | ASVKGRVTISRDDSRGIAYLQ | | GSGSGTDFTLTISGLQPDDFA | |
| | MNSLKTEDTAFYYCTRLRLYR | | TYYCQQANTFPFTFGPGTKVD | |
| | SGWYIEYYFDYWGQGTLVTVS | | IK | |
| | S | | | |
| AB-000361 | EVQLLQSGGGLVQPGGSLRLS | 338 | SYVLTQPPSLSVAPGQTARIS | 799 |
| | CAASGFSFNNYAMTWVRQAPG | | CDGDNIGSKSVHWYQQKPGQA | |
| | MGLEWVSSTTNSGGRTYYADS | | PVLVVYDDNDRPSGIPERFSG | |
| | VKGRFTISRDNSKNTLFLQMN | | SNSGNTAILTISRVEAGDEAD | |
| | SLRAEDTAVYYCAKDGVRASY | | YSCQVWDSSSAHQVFGGGTKL | |
| | DPWGSDYWGQGTLVTVSS | | TVL | |
| AB-000362 | EVKLVESGGGLVKPGGSLRLS | 339 | EIVMTQSPGTLSVSPGERATL | 800 |
| | CAGSGFTFNRYNMNWVRQAPG | | SCRASQSVSSSLAWYQQTLGQ | |
| | KGLEWVSSISSSSSYKHYAVS | | APRLLIYGASTRATGIPARFS | |
| | VEGRFTVSRDNARNSVYLQMN | | GSGSGTEFTLTISSLQSEDYA | |
| | SLRDEDTAVYYCARPLFEITM | | MYYCQQYNNWPYSFGQGTKLE | |
| | VRGVLMTVDAFDIWGQGTMVT | | IK | |
| | VSS | | | |
| AB-000363 | QEQLVQSGTEVKKPGASVKVS | 340 | DIQMTQSPSALSASVGDRVTI | 801 |
| | CKTSGYTFTDYGVSWVRQAPG | | TCRASQTIGNYLNWYQQKPGK | |
| | HGLEWMGWIRPYDGNTNYAQK | | APRFLIYDASSLQTGVPSRFS | |
| | VQGRVTMTTDTSTSTAYMELR | | GTGSGTDFSLTISSLQPEDFA | |
| | SLRSDDTAIYYCARLSGDGRF | | TYYCQQSYDTPYTFGQGTKLE | |
| | YHGMDVWGQGTTVTVSSP | | IR | |
| AB-000365 | QLQLQESGPGLVKPSETLSLT | 341 | QVELTQSPSASASLGASVKLT | 802 |
| | CTVSGGSISSLSHYWGWVRQP | | CTLSSGHSTLAIAWHQQLPEK | |
| | PGKGLEWIGSIYYSGTTYYKP | | GPRYLMRVNSDGSHSKGSGIP | |
| | SLKSRVTISVDTSRNQLSLKL | | DRFAGSSSGAERYLTISSLQS | |
| | NFVTAADTAVYYCARLTGYRD | | EDEGDYYCQTWDTGIAVFGGG | |
| | GWYIYWYFDLWGRGTLVSVSS | | TQVTVL | |
| AB-000370 | QVQMVQSGAEVKKPGASVKVS | 342 | DIVMTQSPDSLAVSLGERATI | 803 |
| | CKASGNTFTAHYLHWVRQAPG | | NCKSSQSVLYTSNNKNYLTWY | |
| | QRFEWMGWINPNSGGTYYPQK | | QQKPGQPPKVVIYWASTRESG | |
| | FQGRVTMTRDTSFTTVYMELR | | VPDRFSGSGSGTDENLTISSL | |
| | RLRSDDTAVYYCARESGAGST | | QAEDVAVYYCQQYYHTRTFGQ | |
| | HFDYWGQGTLVTVSS | | GTKVEIK | |
| AB-000371 | EVQLEESGGGLAQPRGSLRLS | 343 | ETAMTQSPATLSVSPGGRATL | 804 |
| | CVGSGLTFSTYTMNWVRQAPG | | TCRASQNVGSNLAWYQRKPGQ | |
| | KGLEWISYISTNGATIHYADS | | APRLLIYGASTRPTGIPARFR | |
| | VKGRFTISRDNAKNSLYLHMN | | GSGSGTEFTLTISSLQSEDFA | |
| | SLRDDDTAVYHCATTGFGSGS | | VYFCQQYNNWPLTFGGGTKVE | |
| | YWYFDLWGRGTLVTVSS | | IK | |
| AB-000372 | QIQLQQSGPGLVKPSQTLSIT | 344 | DIVMTQSPLSLSVTPGEPASI | 805 |
| | CAISGDSVSNNHAGWNWIRQS | | SCRSSQSLLQTNGNTYLDWYL | |
| | PSRGLEWLGRTYYRSRWYNDY | | QKPGQSPQLLITYNGNRASGV | |
| | AVFVESRITITADTFKNEVSL | | PDRFSGSGSGTDFTLTISRVQ | |
| | QLTSVTPEDTAVYYCTRVKEL | | ADDVGVYYCMQAKETPFTFGG | |
| | GRGFQYWGQGSLITVSS | | GTKVEIK | |
| AB-000374 | AVQLVESGGGVVRPGGSLRLS | 345 | QSALTQPPSASGSPGQSVSIS | 806 |
| | CVASGFTFDDFAMSWVRQVPG | | CTGANSDVGGYNYVSWYQQHP | |
| | KGLEWVSGLNSEGLKSGYADS | | GKAPKLLIYEVNKRPSGVPDR | |
| | VRGRFTISRDNAKNSLYLEMN | | FSGSKSGNTASLTVSGLRAED | |
| | SLRVEDTAFYYCARGASGGWY | | EADYYCNSYAGSNIWVFGGGT | |
| | FDSEFWGQGALVTVSS | | KLTVL | |
| AB-000375 | QVQLVQSGPEMKKPGASVKIS | 346 | DIQMTQSPSTLSASVGDRVTV | 807 |
| | CKASGYQNNNYAIYWVRQAPG | | TCRASENINGWLAWYQQKPGK | |
| | QRPEWMGWINAGNGNTQYSQR | | APKLLIYQASTLESGVSSRFS | |
| | FRGRVTITTDASTRTSYMDLS | | GSGSGTQFTLTISRLQSDDVA | |
| | GLRSADTAVYYCASHTPNSVT | | TYYCQHYKGYSQYTFGGGTRV | |
| | AYDPFHYWGQGTLVTVSS | | EIR | |
| AB-000377 | QVQLQASGPGLVKPSETLSLT | 347 | QSVLTQPPSVSGAPGQTVIIS | 808 |
| | CSVSGDSIRSSSFWGWIRQPP | | CTGSSSNIGAGYDVHWYQQLP | |
| | GKGLEWIGSIYFSGSTYHNPS | | GAAPKLLIYESANRPSGVPDR | |
| | LKSRVTMSVDMSRNQFSLMLS | | FSGSKSGTSASLAITGLQTED | |
| | SVTAADTAVYYCARQRFGSGW | | EADYYCQSYDSGLSGSVFGGG | |
| | ALFYYYYYMDVWGQGTTVTVS | | TKLTVL | |
| | S | | | |
| AB-000378 | EVQLVESGGGFVQTGGSLRLS | 348 | DIQMTQSPSTLSASVGDRVTI | 809 |
| | CVASGFTFSDYEMNWVRQAPG | | TCRASQSITTWLSWYQQKPGN | |
| | KGLEWVSYISSGGGTTKYAES | | APKLLIYEASNLGFGVPSRFS | |
| | VKGRFTISRDNAKNSVFLQMH | | GSGSGTEFTLTITSLQPDDFA | |
| | SLRADDTAIYYCARESALQFN | | TYYCQQHYSYPLSFGGGTKVE | |
| | TILFFDFWGQGTLVTVSS | | IK | |
| AB-000379 | QVQIVQSGAEVKKPGASVKVS | 349 | EIVLTQSPATLSLSPGERATL | 810 |
| | CKTSGYTFTSRNMHWVRQAPG | | SCRASQSVSRYLAWYQQKPGQ | |
| | QRPEWVGWINPDSGDTKYAQW | | APRLLIYDVFDRVTGIPARES | |
| | LQDRVTLSRDTSASTAYMDLR | | GSGSGTDFTLTISSLEPEDSA | |
| | SLRSEDTATYYCVREAGGGTE | | VYYCQQRGNWPLYTFGQGTKL | |
| | SYFVHWGQGTLVTVSS | | EIK | |
| AB-000380 | EMQLLESGGGLVNPGGSLRVS | 350 | QPVLTQPTSLSASPGTSARFT | 811 |
| | CAASGFTFSQAWMSWVRQAPG | | CTLRSDIDFVGFRIYWYQQKP | |
| | KGLEWVGRIESTTDGGTTQYA | | GSLPRYLLRYREDSGVERGSG | |
| | APVKGRFTISRDDSKSSLFLQ | | VSSRFSGSKDASTNVGLLIIS | |
| | MNSVRAEDTAVYYCTTAFGNY | | GLQFEDEADYYCGMWYSKTSE | |
| | NKGHVYVYPYMDVWGKGTTVT | | WLIGGGTKLTVL | |
| | VSS | | | |
| AB-000381 | GVQLVQSGAEVKKPGATVKIS | 351 | DIVMTQSPDSLAVSLGERASI | 812 |
| | CKISGYTFIDYHMHWVKQAPG | | NCRSSQSVLYNSNNKNYLAWY | |
| | KGLEWMGLVDPEDGERIYAKK | | QQKPGQPPKLLIYWASTRESG | |
| | FQGRVIITADTSTDTAYMELS | | VPDRFSGSGSGTDFTLTISSL | |
| | SLRSDDTALYYCATFEFGGSD | | QAEDVAVYHCQQYYSTPVTFG | |
| | AGGFWGQGTLVTVSST | | GGTRVEIK | |
| AB-000382 | QVQLVESGGGVVQPGRSLRVS | 352 | EVVLTQSPGTLSLSPGERATL | 813 |
| | CAASGFSFSTYGMHWVRQAPG | | SCRATQSFSSSYLIWYQQKPG | |
| | KGLEWVALIWYDGSSKYSADS | | QAPRLLIYGASSRATGIPDRF | |
| | VKGRFTISRDNSKNTLFLQMN | | SGSGSGTDFTLTISRLEPEDF | |
| | RLRADDTAVYYCARDLEPTIR | | AVYYCQHHGPSPPYTFGQGTK | |
| | GISRDHHYYGMDVWGQGTTVT | | LEIR | |
| | VSS | | | |
| AB-000383 | EEQLLDSGGGLVQPGGSLRLS | 353 | DIQMTQSPSSLSASVGDRVTI | 814 |
| | CAASGFTFRNYAMTWVRQAPG | | TCRASQSISTHLNWYQQKPGK | |
| | KGLEWVSSITTTGDRATYADS | | APNLLIFDASSLQRGVPSRFS | |
| | VKGRFTISRDNSKNTLFLQMN | | GVGSGTEFTLTISGLQPEDFA | |
| | SLRVEDTAVYYCAKDFPRYNS | | TYYCQQSYTTPRTFGQGTKVE | |
| | GWYANDHNYFGSWGQGTLVTV | | VK | |
| | SS | | | |
| AB-000384 | QVQLVESGGGVVQPGTSLRLS | 354 | SYELTQPPSVSVSPGQTARIT | 815 |
| | CATSGFTFSKYGMHWLRQAPG | | CSGDALPKQYAYWYQQKPGQA | |
| | KGLEWVAVISYDGNHKYYSDS | | PVLVMYKDLERPSGIPERFSG | |
| | VKGRFTISRDNSKNTLYLQLN | | SSSGTTVALTISGVQAEDEAD | |
| | SLRGEDTAVFFCAKLAGTTHS | | YYCQSADSSGTYVIFGGGTKL | |
| | FAYWGQGTLVTVSS | | TVLS | |
| AB-000385 | QLQLQESGPGLVKPSETLSLT | 355 | AIQLTQSPSSLSASVGDRVTI | 816 |
| | CTVSGGSIRSISDWWGWIRQP | | TCRASQDISSSLAWYQQNPGK | |
| | PGKGLEWIGNIYYTGSTNYSP | | APKLLIYAASTLQSGVPSRFS | |
| | SLRSRVIISADMPKNQFSLRL | | GSGSGTDFTLTISSLQPEDFA | |
| | SSVTAADTAVYYCARQVAYRD | | TYYCQHLNSYPLFTFGPGTKV | |
| | GWYVRWYFDLWGRGTRVTVSS | | DIK | |
| AB-000387 | QVQLVQSGSEVKKPGASVRVS | 356 | HSALTQPPSASGSPGQSVTIS | 817 |
| | CKASGYFFNYYYIHWVRQAPG | | CSGTSSDVGGYNYVSWYQQHP | |
| | QGLEWMGWINPNSGDTDSVKK | | GKAPKLIIYEVNKRPSGVPDR | |
| | FQGRVTMTRDTSISTAYMDLR | | FSGSKSGSTASLTVSGLQAED | |
| | SLTSVDTAVYYCARLNRGYTH | | EADYYCSSYAGSNNFVFGGGT | |
| | GSALDFWGQGTIVTVSS | | KLTVL | |
| AB-000388 | QVQLVQSGAEVKKPGASVKVS | 357 | QSVLTQPPSVSAAPGQRVTIS | 818 |
| | CKTSGYSFTINGISWVRQAPG | | CSGSSSNIGNNRVSWYQHLPG | |
| | QGLEWVGWMSANSGNTIYAQK | | TAPKLLIYDNDQRPSGIPDRF | |
| | FQSRVTMTRNTSTSTAHMELR | | SGSKSGTSATLGITGLQTGDE | |
| | SLRSDDTAIYYCARDRNYALE | | ADYYCGTWDSSLSDGRVFGGG | |
| | SWGQGTLVTVSS | | TKVTVL | |
| AB-000392 | QVQLVESGGGVVQPGRSLRLS | 358 | SYELTQPSSVSVSPGQTARIS | 819 |
| | CAASGFTFSSYAMHWVRQAPG | | CSGPLLSKNFARWFQQKPGQA | |
| | KGLEWVAIISYDGSKKYLADS | | PILVIYKDTARPSGIPERFSG | |
| | VKGRFTISRDNSKNTLYLQMN | | SSSGTTVTLTISGAQVEDEAD | |
| | SLRSEDTAVYYCASEPDTAYI | | YYCYSATDNNLGVFGGGTKLT | |
| | VDHWGQGTLVTVSS | | VL | |
| AB-000396 | QLQLQESGPGLVKPSETLSLT | 359 | EIVLTQSPGTLSLSPGKRATL | 820 |
| | CSVSGGSMRSSSYYWAWIRQP | | SCRASQSISSKYLAWYQQKPG | |
| | PGKGLEWIGSIYNSGTTYYHP | | QAPRLLIHAASSRATGIPDRF | |
| | SLESRVTMSVDTSMNQFSLKL | | SGSGSGTDFTLTISRLEPEDE | |
| | SSVNAADTAVYYCARPAAVDY | | GVYYCQQLGSSPFTFGPGTTV | |
| | SLTSYYPGEGAFDVWGQGTMI | | DIK | |
| | TVSS | | | |
| AB-000398 | EVQLVESGGGLVQPGGSLRLS | 360 | SYELMQPPSVSVSPGQTARIT | 821 |
| | CGASGFTFSNFAITWVRQAPG | | CSGDALPKRYAYWYQQKSGQA | |
| | KGLEWVSVISGSGGRTYYADS | | PVLVIYEDSKRPSGIPERFSG | |
| | LKGRFIISRDNSKNMVYLQMN | | SSSGTMATLTISGAQVEDEAD | |
| | SLRAEDTGVYYCAKNKNDKSG | | YYCYSTDSSGSDRRDVFGTGT | |
| | QYPLYYWGQGALVTVSS | | KVTVL | |
| AB-000400 | QVQLQESGPGLVEPSGTLSLT | 361 | QSVLTQPPSVSEAPGQRVTIS | 822 |
| | CAVSGASISNFHWWSWIRQPP | | CTGSSSNIGAGSDVHWYQQLP | |
| | GKGLEWIGQIHRDGSTKYNPN | | GTAPKLLISANSNRPSGVPDR | |
| | PSLKSRVTVSLDKSNNNFSLR | | FSGSKSGTSASLVITGLQPED | |
| | LTSVTAADTAVYYCAAEDGSG | | EADYYCHAYDTSLSASVFGGG | |
| | TNWFDPWGQGNLVTVSS | | TKLTVL | |
| AB-001389 | QVQLQESGPGLVKPSETLSLT | 362 | QSVLTQPPSVSAAPGQKVTIS | 823 |
| | CTVSGDSISSSNYYWGWIRQP | | CSGSSSNIGNTYVSWYQQLPG | |
| | PGKGLDYIGSIHNSGRTSYNP | | TAPKLLIYDNDKRPSGIPDRF | |
| | SLKSRVTVSVDTSKNQFSLKL | | SGSKSGTSATLGITGLQTGDE | |
| | TSVTAADTAVYYCARQGSYSS | | ADYYCGTWDTSLSAGVFGGGT | |
| | GWYPFDFEDYWGQGVLVTVSS | | KLTVL | |
| AB-001391 | QVQLVQSGAEVKKPGASVKVS | 363 | NFMLTQPHSVSESPGKTVTIS | 824 |
| | CKASGYTFTIYDISWVRQAPG | | CTRSSGSIASNYVQWYQQRPG | |
| | QGLEWMGWISAYNGHTNYAQK | | SSPTIVIYEENQRPSGVPDRF | |
| | LQGRVTMTTDTSTSTAYMELR | | SGSIDSSSNSASLTISGLKTE | |
| | SLRSDDTAVYYCARGYRKSSS | | DEADYYCQSYDSSTVVFGGGT | |
| | GWYWNNWYFDLWGRGTLVTVS | | KLTVL | |
| | S | | | |
| AB-001394 | QVTLKESGPVLVKPTETLTLT | 364 | QSALTQPASVSGSPGQSITIS | 825 |
| | CTVSGFSLNNARMGVSWIRQP | | CSGTSSDVGSYNLVSWYQQHP | |
| | PGKALEWLAHIFSNDEKSFST | | GKAPKLMIYEGSERPSGVSSR | |
| | SLKSRLTISKDTSKGQVVLTL | | FSGSKTDHTASLTISGLQAED | |
| | TNMDPVDTATYFCARIPHRSG | | EAHYFCCSYARSRTWVFGGGT | |
| | SYQPYYYYYGMDVWGQGTTVT | | KLTVL | |
| | VSS | | | |
| AB-001397 | EVQLVESGGGAIQPGGSLRLS | 365 | QSALTQPASVSGSPGQSITIS | 826 |
| | CVASGFRVNWNYMTWVRQAPG | | CTGTSSDVGAYNYVSWYQQHP | |
| | KGLEWVSVIHIDGTTYYADSV | | GEAPKLIIYEVTNRPSRVSDR | |
| | KGRFTISRDNSNNLLFLQMNN | | FSGSKSGNTASLIISGLQPGD | |
| | VRAEDTAVYFCARDTDSWWRF | | EADYYCSSYTSKTTLAVFGTG | |
| | DFWGQGTLVTVSS | | TKVTVVS | |
| AB-001401 | QVQLVQSGAEVKKPGSSVKVS | 366 | DIQMTQSPSSLSASIGDRVTI | 827 |
| | CKASGGTFSSYAISWVRQAPG | | TCRASQGISNHLAWFQQKPGK | |
| | QGLEWMGGIIPIFGVTKYTQK | | APKSLIYAASSLQTGVPSKES | |
| | FQGRVTITADRSTNTAYMELS | | GSGSGTDFTLTITSLQPEDFA | |
| | SLRSEDTALYYCASRTIVVAL | | TYYCQQYNSYPYTFGQGTKLD | |
| | NYYYGMDVWGQGTTVTVSS | | IK | |
| AB-001406 | EVQLLESGGGLVQPGGSLRLS | 367 | EIVLTQSPATLSLSPGERATL | 828 |
| | CAASGFTFSFYAMSWVRQAPR | | SCRASQSVSSYLAWYQQKPGQ | |
| | KGLEWVSTISTSGDITYYADS | | APRLLIYDASNRATGIPARFS | |
| | VKGRFTISRDNSKNTLYLQMN | | GSGSGTDFTLTISSLEPEDFA | |
| | SLRAEDTAVYYCATNTPLKSS | | VYYCQQRSNWPHTFGPGTKVD | |
| | GWYISGFGYFDYWGQGTLVTV | | IK | |
| | SS | | | |
| AB-001412 | QLQLQESGPGLVKPSETLSLT | 368 | QSALTQPASVSGSPGQSITIS | 829 |
| | CTVSGGSISSSSHYWGWIRQP | | CTGTSSDVGGFKYVSWYQQHP | |
| | PGKGLEWIGRIYYSGSTSYNP | | GKAPKLMIFDVSKRPSGVSNR | |
| | SLKSRVTISIDTSKNQFSLKL | | FSGSKSGNTASLAISGLQAED | |
| | NSVTAADSAVYYCARDMIYSS | | EADYYCSSYTTFSSYVVFGGG | |
| | SWYLVAGNFDSWGQGTLVTVS | | TKLTVL | |
| | S | | | |
| AB-001414 | QVQLVQSGAEVKKPGASVKVS | 369 | QSALTQPASVSGSPGQSITIS | 830 |
| | CKASGYSFSSYGISWVRQAPG | | CTGTSSDVGYYNLVSWYQQHP | |
| | QGLEWMGWISGYTGKTNYAQK | | DKAPKLMIFEDSKRPSGVSNR | |
| | FQGRVTMTTDTATSTAHMELR | | FSGSKSGNTASLTISGLQADD | |
| | SLRSDDTAIYYCARAPHAKNN | | EADYYCSSHAHNGIYVLFGGG | |
| | GWYVEGPGAFDFWGQGTTVTV | | TKLTVL | |
| | SS | | | |
| AB-001416 | DVQLVESGGGLVQPGESLRLS | 370 | SYELTQPPSVSVSPGQTATIT | 831 |
| | CTASGFTLSTYWMSWVRQAPG | | CSEDKLGDKYTYWYQQKPGQS | |
| | KGLEWVAKIKEDGSEKYYLDS | | PVLVIYQDNKRPSGIPERFSG | |
| | VKGRFTMSRDNAKNSLDLQMN | | SSSRNTATLTISGTQPMDEAD | |
| | SLRAEDTAVYYCARDSTGGFD | | YHCQAWDSSAVVFGGGTKLTV | |
| | YWGRGTLVTVSS | | LS | |
| AB-001417 | EVQMVESGGGLVQPGGSLRLS | 371 | QSVLTQPPSVSGAPGQRVTIS | 832 |
| | CAASGFTFSSYEMNWVRQAPG | | CTGSSSNIGAGYDVHWYQQLP | |
| | KGLEWVSYISSSGSTKYYADS | | GTAPKLLIYGNTNRPSGVPDR | |
| | VKGRFTISRDNAKNSLYLQMN | | FSGSKSGTSASLAITGLQAED | |
| | SLRAEDTAVYFCARDTMLWTY | | EADYYCQSYDRGLRVFGTGTK | |
| | YFDNWGQGTLVTVSS | | VTVL | |
| AB-001418 | QVQLVQSGAEVKKPGASVKVS | 372 | QSVLTQSPSASGTPGQRVTIS | 833 |
| | CKASGYTFTSYDIHWARQATG | | CSGSSSNIGTNTVTWYQQVPG | |
| | QRLEWMGWLSPNTGNTGYAQK | | TAPKVLIYSNNQRPSGVPDRF | |
| | FQGRVILTRNTSINTAYMEVT | | SGSKSGTSASLAISGLQSEDE | |
| | SLRFQDAAVYYCARGHTGYSN | | ADYYCAAWDDSLTVVFGGGTK | |
| | GWDSWLDPWGQGTLVTVSS | | LTVL | |
| AB-001419 | QVQLVESGGGVVQPGRSLRLS | 373 | SSELTQDPAVSVALGQTVRIT | 834 |
| | CAASGFTFSSYIMHWVRQAPG | | CQGDSLRSYYASWYQQKPGQA | |
| | KGLEWVAVISYDGSNKYYADS | | PVLVIYAKNNRPSGIPDRFSG | |
| | VKGRFTISRDNSKNTLYLQMN | | STSGNTASLTITGAQAEDEAD | |
| | SLRAEDTAVYYCAREDPGRFG | | YYCNSRDSSGNHYVFGTGTKV | |
| | ELLVQLGGGRNYYYYGMDVWG | | TVL | |
| | QGTTVTVSS | | | |
| AB-001420 | QVQLVQSGAEVKKPGASVKVS | 374 | EIVLTQSPGTLSLSPGERATL | 835 |
| | CKASGYTFINYGISWVRQAPG | | SCRASQSVSSADLAWYQQKPG | |
| | QGLEWMGRINAYSGYTNLPQK | | QAPRILIYGASSRATGIPDRF | |
| | LQGRVTMTTDTSTSTVYMELR | | SGSGSGTDFTLTISRLEPEDF | |
| | SLRSDDTAVYYCARDAPTYSS | | AVYYCQQYASSPRTFGQGTKV | |
| | GWYRHYYYMDVWGKGTTVTVS | | EIK | |
| | S | | | |
| AB-001422 | EVQLVESGGGLVKPGGSLRLS | 375 | QSALTQPVSVSGSPGQSITIS | 836 |
| | CAASGFTFNTYNMNWVRQAPG | | CTGTSSDIVNYHYVSWYQVHP | |
| | KGLEWVSSISSSSSYINYAES | | GKAPKLMIYEVSNRPSGVSDR | |
| | VKGRFTISRDNVKKSLYLQMN | | FSGSRSANTASLTISGLQAED | |
| | SLRAEDTAVYYCAGQRITLGR | | EADFYCSSYTNSGTLVFGAGT | |
| | GAITAAPYLDHWGQGTRVAVS | | KVTVLS | |
| | S | | | |
| AB-001423 | QLQLQESGPGLVKPSETLSLS | 376 | DIVLTQSPATLSLSPGERATL | 837 |
| | CTVSGGSIKSPSFYWGWIRQP | | SCRASQSISTYLAWYQQRPGQ | |
| | PGKGPEWIGSFYYSGTTFYSP | | PPRLLIYNASNRATGIPARFS | |
| | SLKGRVTISTDTSKNQFSLRL | | GSGSGTDFTLTISSLEPEDFA | |
| | SSVTAADTAVYFCAREFLYMS | | VYYCQQRTNWPPGRTFGQGTK | |
| | DLLVGQAFDIWGQGTMVTVSS | | VEIK | |
| AB-001424 | EVQLVESGGGLVQPGGSLRLS | 377 | ETVMTQSPDSLSVSPGARATL | 838 |
| | CAASGFPFSAYTMSWVRQAPG | | SCRTSHSVQSNLAWYQRKPGQ | |
| | KALEWISYISTTSDTIYYADS | | PPRLLIYGASTRATGVPARFS | |
| | VKGRFTISRDNAKNSVSLQMK | | GSGSGTEFTLTISGLQSEDFA | |
| | SLRVEDSALYFCARVAFGSGS | | VYSCQQYNNWPLTFGGGTKVE | |
| | YWFFDYWGQGILVTVSS | | IR | |
| AB-001426 | QVQLVESGGGVVQPGRSLRLS | 378 | DIQMTQSPSTLSASVGDRVTI | 839 |
| | CAASGFTFSSYGMHWVRQAPG | | TCRASQSISSWLAWYQQKPGK | |
| | KGLEWVAVIWYDGSNKYYADS | | APKLLIYKASSLESGVPSRFS | |
| | VKGRFTISRDNSKNTLYLQMN | | GSGSGTEFTLTISSLQPDDFA | |
| | SLRAEDTAVYYCARPGDRGTD | | TYYCQQYNSQYTFGQGTKLEI | |
| | HTDAFDIWGQGTMVTVSS | | K | |
| AB-001427 | EEQLVESGGGLVQPGGSLRLS | 379 | DIQMTQSPSSLSASVGDRVTI | 840 |
| | CAASGFTFSSYEMNWVRQAPG | | TCRASQSISSYLNWYQQKPGK | |
| | KGLEWVSDISSSGSTIYYADS | | APKLLIYAASSLHSGVPSRFS | |
| | VKGRFTISRDNAKNSLYLQMN | | GSGSGTDFTLTISSLQPEDFA | |
| | SLRAEDTAVYYCARGELDLGA | | TYYCQQSYSTPWTFGQGTKVE | |
| | FDIWGQGTMVTVSS | | IK | |
| AB-001430 | QVQLVESGGGVVQPGRSLRLS | 380 | DIVMTQSPDSLAVSLGERATI | 841 |
| | CAASGFTESSYGMHWVRQAPG | | NCKSSQSVLYSSNNKHYLAWY | |
| | KGLEWVAVIWYDGSNKYYADS | | QQKPGQPPKLLIYWASTRESG | |
| | VKGRFTISRDNSKNTLYLQMN | | VPDRFSGSGSGTDFTLTISSL | |
| | SLRAEDTAVYYCTAATQYGLL | | QAEDVAVYYCQQYYSTWTFGQ | |
| | GGAFDYWGQGTLVTVSS | | GTKVEIK | |
| AB-001431 | EVQLVQSGGGQVQPGESLRLS | 381 | DVQMTQSPTFLSASVGDRVTI | 842 |
| | CAASGFTFTSYWMSWVRQAPG | | TCQASQGINNYLNWYQQKPGK | |
| | KGPEWVANINHAGSQKYYVDS | | APKLLIYDAFNLEVGVPSRFS | |
| | VKGRFTISRDNAKNSLELQMN | | GSGSGTEYTFTISSLQPEDIA | |
| | SLRAEDTAVYYCARFWRVLPD | | TYYCQQYDNFPTFGQGTKVEI | |
| | YFDTSGRAVDYWGQGILVTVS | | K | |
| | S | | | |
| AB-001432 | QVQLVQSGAEVKKPGASVKVS | 382 | EIVLTQSPATLSLSPGERATL | 843 |
| | CKASGYSFTTHGISWVRQAPG | | SCRASQSVSSYLAWYQQKPGQ | |
| | QGLEWMGWISAYSGNTNYAQK | | APRLLIYDTSNRATGIPARFS | |
| | LQGRVTMTTDTSTSTAYMELR | | GSGSGTDFTLTISSLEPEDFA | |
| | SLRSDDTAVYYCARDKLLWER | | VYYCQQRSNWPITFGQGTRLE | |
| | ELPHLPRDNWFDPWGQGTLVT | | IK | |
| | VSS | | | |
| AB-001433 | QVQLQQWGAGLLKPSETLSLT | 383 | SSELTQDPSVAVAVGQTVRIT | 844 |
| | CAVYGGSFSDYYWTWIRQPPG | | CRGTSLRTYYASWYQQKPGQA | |
| | KGLEWIGEINHSGRTNYKSSL | | PLLVIYGKNTRPSGIPDRFSG | |
| | RSRVTISVDTSKNQFSLKLTS | | SSSDNMASLTITGAQAEDEAD | |
| | VTAADTAMYYCARGQIQISMM | | YFCNSRDSSGRPFFGGGTKVT | |
| | AVIFTGGAYYFDSWGQGTLVA | | VL | |
| | VSS | | | |
| AB-001436 | QVQLVESGGGVVQPGRSLRLS | 384 | DIVMTQSPDSLAVSLGERATI | 845 |
| | CAASGFTFSSYGMHWVRQAPG | | NCKSSQSVLYSSNNKNYLAWY | |
| | KGLEWVAVIWYDGSNKYYADS | | QQKPGQPPKLLIYWASTRESG | |
| | VKGRFTISRDNSKNTLYLQMN | | VPDRFSGSGSGTDFTLTISSL | |
| | SLRAEDTAVYYCARPGFFSTY | | QAEDVAVYYCQQYYSTWTFGQ | |
| | YEDRGDAFDIWGQGTMVTVSS | | GTKVEIK | |
| AB-001442 | QVQLQESGPGLVKPSGTLSLT | 385 | EIVLTQSPATLSMSPGERATL | 846 |
| | CDVSGASITSTKNWCSWVRQS | | SCRASQSVSSYLAWYQQKPGQ | |
| | PGKGLEWIGEIYHSGDTNYNP | | APRLLIYDASNRATGIPARES | |
| | SLKSRVTMSVDTSKNQFSLKL | | GSGSGTDFTLTISSLEPEDSA | |
| | SSVTAADTAVYYCARDMRIPA | | VYYCQQRENWPVYTFGQGTRL | |
| | RPDQGHYFDFWGQGTLVTVSS | | EIR | |
| AB-001444 | AEQLLESGGGLVQPGESLRLS | 386 | QSALTQPPSASGSPGQSVSIS | 847 |
| | CAASGFTFGSYGLSWVRQVPG | | CTGTSSDVGGYNYVSWYQQHP | |
| | KGLEWVSHIRVRGSGTSYADS | | GQAPKLIIYDVSKRPSGVPDR | |
| | VKGRFAISRDNSNNMLYLQMS | | FSGSKSGNTASLTVSGLQAED | |
| | SLRAEDTAVYYCAKSTGWLQF | | EADYYCSSYAGNKNTVFGGGT | |
| | DCWGQGTLVTVSS | | KLTVL | |
| AB-001445 | QVHLLELGPRLVKPSETLSLT | 387 | AIVLTQSPATVSLSPGERATL | 848 |
| | CAVSGYSVGSGYYWGWIRQPP | | SCRAFQSAINNDIAWYQQKPR | |
| | GKGLEWVGTIYFTENTSFGPS | | QAPRLVMYGTSNRATGIPDRF | |
| | FGGRATISVDASENTFSLTLD | | RGSGSGADFTLTITRLEPEDY | |
| | SVTAADTAMYYCAARTAVVNE | | AVYYCQHYGRSKTFTFGPGTR | |
| | YYFDDWGQGILVTVSS | | VELK | |
| AB-001447 | QLQLQESGPGLLRPSETLSLT | 388 | QLMLTQSPSASASLGASVKLT | 849 |
| | CTVSGGSTSRGSYYWGWIRQP | | CTLSSGHSTYAIAWLQQRPEK | |
| | PGKGLEWIGSIYYSGSTYSNP | | GPRYLMKLNSDGSHDKGDGVP | |
| | SLKSRVTMSLDTSKNQFSLKL | | DRFSGSSSGAERSLTISSLQS | |
| | SSVTAADTAVYYCARHSPYSS | | EDEAEYYCQAWGTGIHGVFGA | |
| | GWYIHFYFDSWGQGTLVTVSS | | GTRLTVL | |
| AB-001453 | EVQLVESGGGLVQPGGSLRLS | 389 | SYELTQPPSVSVSPGQTARIT | 850 |
| | CAASGFTFSSYWMHWVRQAPG | | CSGDALPKKYAYWYQQKSGQA | |
| | KGLVWVSRINSDGSYTSYADS | | PVLVIYEDSKRPSGIPERFSG | |
| | VKGRFTISRDNAKNTLYLQIN | | SSSGTMATLTISGAQVEDEAD | |
| | SLRAEDTAVYYCARVRYYYDS | | YYCYSTDSSGNGVFGGGTKLT | |
| | SGSKGGYYYYMDVWGKGTTVT | | VL | |
| | VSS | | | |
| AB-001454 | QVQLVQSGAEVKKPGASVKVS | 390 | DIQMTQSPSSLSASVGDRVTI | 851 |
| | CQSSGDVFTTYGFTWVRQAPG | | TCRASQFISSYLNWYQQKPGK | |
| | QGLEWMGWISTYKDDAEYSQR | | APKILIYAASTLQSGVPSRFS | |
| | FQGRVTMTTDTSSSTAYMELR | | GSGSGTDFTLTISSLQPEDFA | |
| | SLRSGDTAVYYCARGSKWELL | | TYYCQQSSSSPRTFGGGTRVE | |
| | HWGQGTPVTVSS | | IK | |
| AB-001456 | QVTLKESGPALVKPTQTLTLT | 391 | DIQMTQSPSSLSASVGDRVTI | 852 |
| | CTFSGFSLSTTGVRVSWIRQP | | TCRASQSISNYLNWYQQKPGK | |
| | PGKALEWLARIDWDDDKFYST | | VPKLLIYVASSLQSGVPLRFS | |
| | SLRTRLTISKDTSKNQVVLTM | | GSGSGTDFTLTISSLQPEDFA | |
| | TNMDPVDTATYFCARLKYDNS | | TYYCQQSYNAPLTFGQGTKVE | |
| | GRGDYWGQGTLVTVSS | | IK | |
| AB-001461 | EVQLVESGGGLVQPGGSLRLS | 392 | QSALTQPASVSGSPGQSITIS | 853 |
| | CAASGFTFIDHWMHWVRQVPG | | CTGTSSDVGNYNYVSWYQQVP | |
| | KGLVWVCRISTDGSSTTYADF | | GKAPELLLYDVNNRPSGISNR | |
| | VKGRFAISRDNAKNTMSLQMR | | FSGSKSGNTASLTISGLQADD | |
| | GLRAEDTAVYYCVRGSRFYFG | | EADYYCSSYSGSFTPYVFGTG | |
| | SGSSDYLGQGTLVTVSS | | THVTVQ | |
| AB-001463 | QVQLEESGGGLVKPGGSLRLS | 393 | EIVLTQSPDTLSLSPGERATL | 854 |
| | CAASGFTFSYYYMSWIRQAPG | | SCRASQSVTRSYLAWYQQKPG | |
| | KGLEWVSYISSDGRIIYYADS | | QAPRLLISGASSRATGVPDRF | |
| | VRGRFTISRNNAENSLSLLMN | | SGSGSGTDFTLTISRLEPEDL | |
| | SLRTEDTAVYYCARAAPEYSN | | AMYYCQQYGSSHTFGQGTKLE | |
| | GWYLDGRGAFDIWGQGTMVTV | | IK | |
| | SS | | | |
| AB-001464 | EVQMVESGGGLVQPGGSLRLS | 394 | DFQVTQSPSTLSASVGDRVTI | 855 |
| | CVASGFTFNNHWMHWVRQAPG | | ICRASQSISDWLAWYQQKPGN | |
| | KGLVWVSRISSDGISTSYADS | | APKLLIYKASNLESGVPSRFS | |
| | VKGRFTISRDNAKNTLYMQMN | | GSRSGTEFTLTISSLQPDDFA | |
| | SLRVDDTAVYFCARGKWSGYL | | TYYCQLYNSYYYTFGQGTKVE | |
| | LSHCFDQWGQGTLVTVSS | | IK | |
| AB-001466 | QMQLVESGGGLVKPGGSLRLS | 395 | EIVMTQSPATLSVSPGERATL | 856 |
| | CAASGFTFSDYYMSWIRQAPG | | SCRASQSISSNLAWYQQKPGQ | |
| | KGLEWVSYINSGSTYTNYADS | | APRLLIYGASTRATGIPARES | |
| | VKGRFTISRDNARNSLYLQMH | | GSGSGTDFTLTISSMQSEDFA | |
| | SLRAEDAAVYYCARDYQWREL | | VYSCQQYNHWPLTFGGGTKVE | |
| | NDHWYFDLWGRGTLVTVSS | | IK | |
| AB-001473 | EGQLVESGGGLVQPGGSLRLS | 396 | ETVLTQSPGTLSLSPGESATL | 857 |
| | CTASGFSVSSNYINWVRQVPG | | SCRASQSVRNNFLAWYQQRPG | |
| | KGLEWVSIFYSSGGTDYTDAV | | QAPRLLIYGASSRATGIPDRF | |
| | KGRFTISRDNSKNTVHLQMNS | | SGSGSGTDFTLTISRLEPDDF | |
| | LRVEDTAVYYCAGRAGSARGA | | AVYYCQQYESSPPVTFGPGTR | |
| | FDVWGQGTMVSVSS | | VEIK | |
| AB-001485 | EVQLVEFGGGLVKPGGSLRLS | 397 | QSVLTQPPSASGTPGQRITIS | 858 |
| | CAASGFTFSSYTMNWVRQAPG | | CSGSSSNIGRNTVNWYQQFPG | |
| | KGLEWVSSIGSSGSYMYYAGS | | TAPKLLIHSRNQRPSGVPDRF | |
| | VEGRFTISRDNANNSLYLQMN | | SGSRSGTSASLAISGLQSEDE | |
| | SVRVDDTAVYYCARIAGFEGV | | ADYYCAAWDDSLNGHWVFGGG | |
| | DHWGQGTLVTVSS | | TKLTVL | |
| AB-001487 | EVQLVQSGAEVKKPGESLKIS | 398 | EIVLTQSPGTLSLSPGERATL | 859 |
| | CKGSGYSFTNYWIGWVRQMPG | | SCRASQSVSSNSLAWYQQKPG | |
| | KGLEWMGIIYPGDSDTRYSPS | | QAPRLLIYGASSRATDIPDRF | |
| | FQGQVTISADKSISTAYLQWS | | SGSGSGTDFTLTIGRLEPEDF | |
| | SLKASDTAMYYCARLLYTKIT | | AVYYCQQYGSSPWTFGQGTKV | |
| | LVRGALDYWGQGTLVTVSS | | EIK | |
| AB-001489 | QVQLVQSAAEVRKPGASVKVS | 399 | DIQLTQSPSRLSASIGDRVTI | 860 |
| | CRPSGYRFTDYYLAWVRQAPG | | ACRASQNIDVWLAWYQQRPGK | |
| | QGLEWMGYINPTAHGGTYGQK | | APKLLVYKASTLEPGVPSRFS | |
| | FQGRITLTADPSVSTVYMELT | | GSGSGTEFTLTITSLQPDDVA | |
| | GLQADDTAVYYCVRSRSFYFD | | TYYCQQYESLSDLTFGGGTKV | |
| | QWGQGTLVTVAT | | EIK | |
| AB-001490 | QVQLVQSGSELKKPGASVKVS | 400 | QLVLTQSPSASASLGASVNLT | 861 |
| | CKASGYTFSNHAINWVRQAPG | | CALSSGHSSYAIAWHQQKPEK | |
| | QGLEWMGWIKTNTGNPTYAQG | | GPRYLMKVNSDGSHSKGDGIP | |
| | FTGRFVFSLDTSVSTAYLQIS | | DRFSGSSSGAERYLTISSLQS | |
| | SLKAEDTALYYCARGYISGYH | | EDEADYYCQTWGAGIQEFGGG | |
| | FDYWGQGTLVTVSS | | TKLTVL | |
| AB-001491 | QLHLQESGPGIVKPSETLSLT | 401 | QTVVTQEPSLTVSPGGTVTLT | 862 |
| | CIVSGDSIRTSSYYWGWIRQP | | CASSTGAVTSGHYPTWFQQKP | |
| | PGKGLEWISNRYYRGSTNYSP | | GQAPRALIYSTNDKHSRTPAR | |
| | SLKSRATLSVDTSKNQFSLRL | | FSGSLLGGKAALTLSGVLPED | |
| | TSVTAADTATYYCIREKSFFA | | EADYYCLLFYGGAWEFGGGTK | |
| | EITWGQGTLVTVSS | | LTVL | |
| AB-001494 | EVQLVESGGGLVQPGRSLRLS | 402 | QSVLTQPPSVSAAPGQKVTIS | 863 |
| | CAASGFTFDDNAMHWVRQAPG | | CSGSSSNIGNNYVSWYQQLPG | |
| | KGLEWVSGIGWNSGRIGYADS | | TAPKLLIYDNNKRPSGIPDRF | |
| | VKGRFTISRDNAKNSLYLQMN | | SGSKSGTSATLGITGLQTEDE | |
| | SLRAEDMALYFCAKEYGDYDA | | ADYYCGTWDSSLSAGVFGGGT | |
| | FDIWGQGTMVTVSS | | KLTVL | |
| AB-001496 | QVQMVESGGGVVLPGRSLRLS | 403 | QSALTQARSVSGSPGQSVTIS | 864 |
| | CAASGFTFRNYAMHWVRQAPG | | CTGTSSDVGACNCVSWYQQHP | |
| | KGLEWVAVISYDGSNKYYADF | | GKVPKLIIYDVNKRPSGVPDR | |
| | VKGRFTISRDSSKNTLYLEMN | | FSGSKSGNTASLTISGLQAED | |
| | SLRPEDTAVYYCAKDRVLGVL | | EADYYCCSYAGSYTVLFGGGT | |
| | VPVAALFDYLGQGTLVTVSS | | TLTVL | |
| AB-001497 | QVQLVQSGAEVKKPGSSVKVS | 404 | EIVMTQSPATLSVSPGERATL | 865 |
| | CKASGGTFSSYAISWVRQAPG | | SCRASQSVSSNLAWYQQKPGQ | |
| | QGFEWMGGIIPIFGTANYAQK | | APRLLIYGASTRATGIPARFS | |
| | FQGRVTITADESTSTAYMELS | | GSGSGTEFTLTISSLQSEDFA | |
| | SLRSEDTAVYYCARDSDGDYV | | VYYCQQYNNWPLYSFGQGTKL | |
| | SGAFDIWGQGTMVTVSS | | EIK | |
| AB-001498 | EVQLVESGGGLVNPGGSLRLS | 405 | QSLLTQPPSASGTPGQRVTIS | 866 |
| | CAASGFTENSYSMNWVRQAPG | | CSGSSSNIGTNTVNWYHQLPG | |
| | KGLEWVSSIDTTSHYIYYAHS | | TAPKLLIYSDNQRPSGVPDRL | |
| | VRGRFTVSRDNAKNSLFLQVN | | SGSKSGTSASLVINGLQSEDE | |
| | SLRGEDTALYYCARAHCGGRG | | ADYYCAAWDDSLKGVIFGGGT | |
| | CFQRFDAFDIWGQGTMVTVSS | | KLTVL | |
| AB-001499 | EVQLVQSGADVKKPGESLRIS | 406 | EVVLTQSPATLSVSPGDSATL | 867 |
| | CKGSGYSFPNYSIGWVRQMSG | | SCRASQIVNNNLAWYQQKPGQ | |
| | KGLEWMGNIDPADSETRYSPP | | APRLLIFASSTRATGIPARFS | |
| | FQGQVTMSVDKSISTAYLEWS | | GSGSGTEFTLTISSLQSEDFA | |
| | SLKASDSGIYFCARLRAFVCP | | AYYCQQYNYWPGTFGQGTKVE | |
| | DFWGQGTLVTVSS | | VK | |
| AB-001501 | QVQLQESGPGLVKPSETLSLT | 407 | ETVLTQSPGTLSLSPGETATL | 868 |
| | CTVSGGFISGYYWSWIRQPAG | | SCRASQSINDNSLAWYHQKPG | |
| | KGLEWIGHIYSSGIINDNPSL | | QAPRLLIFGASTRATDIPDRF | |
| | KSRVTMSVDTSKNQFYLNLTS | | SGSGSGTDFTLSISRLEPADF | |
| | VTAADTAVYYCARQGDYGDCF | | GLYFCQQYDRSPVTFGPGTTV | |
| | DNWGQGTLVTVSS | | DVK | |
| AB-001505 | EVQLVESGGGLVQPGGSLRLS | 408 | QSALTQPASVSGSPGQSITIS | 869 |
| | CAASGFTESSYEMNWVRQAPG | | CTGTSSDVGTYNLVSWYQQHP | |
| | KGLEWVSYISSSGSSIYYADS | | GKAPKLMIYEGSKRPSGVSNR | |
| | VKGRFTISRDNAKNSLYLQMN | | FSGSKSGNTASLTISGLQAED | |
| | SLRAEDTAVYYCARDDLLWLY | | EADYYCCSYSVSSTYVFGTGT | |
| | YFDYWGQGTLVTVSS | | KVTAL | |
| AB-001506 | EVQLVESGGGLVKPGGSLRLS | 409 | QSALTQPASVSGSPGQSITIS | 870 |
| | CAASGFTFSTFGMNWVRQAPG | | CTGTNNDIGDYYLVSWYQHHP | |
| | KGLEWVSFISGSSNYIHSADS | | GRAPKLIIYEVTRRPSGTSNR | |
| | VKGRFTISRDNAKNSVYLQMN | | FSGSKSGNTASLTISGLQAED | |
| | SLRAEDTAVYYCVRGGAWLGE | | EADYYCCSFAGGNTFVLFGGG | |
| | FKHPRQFDYWGQGSLVTVSS | | TKVTVL | |
| AB-001507 | EVQLVESGGSVVRPGGSLRLS | 410 | DIQMTQSPSTLSASVGDRVSI | 871 |
| | CAASGFREDDYGMSWVRQAPG | | TCRATQTISNWLAWYQHKPGK | |
| | KGLEWVSRINWNGGVTESADS | | APNLLIYEASDLETGVPSRFS | |
| | VKGRFTISRDNTKNSLYLQMN | | GSGSGTEFTLTISSLQAEDFA | |
| | SLRAEDTALYYCARQAVAGIL | | TYYCQQYKSYPWTFGQGTQVE | |
| | WYFDLWGRGTLVTVSS | | IK | |
| AB-001509 | DVQLLESGGGLVQPGGSLRLS | 411 | EIVLTQSPGTLSLSPGETATL | 872 |
| | CAVSGFTFKNYAMSWVRQAPG | | SCRASQSVGGNYLAWYQHKPG | |
| | SGLVWVSSITTDGESTYYADS | | QAPRLVIYGASTRATGIPDRF | |
| | VRGRFTISRDNSKNTLSLRMN | | SGSGSGTDFTLSISRLEPEDF | |
| | SLGAEDTAVYFCARDHPRYSN | | AVYYCQHYGPSPRTFGQGTKV | |
| | GWYVSAMNYFDSWGRGIPVTV | | EIK | |
| | SS | | | |
| AB-001514 | EVQLVESGGGLVQPGRSLRLS | 412 | DIVMTQSPDSLAVSLGERATI | 873 |
| | CAASGFTEDDYVMHWVRQAPG | | NCKSSQSVLYSENNKNYLAWY | |
| | KGLEWVSGISWYNGSIGYADS | | QQKPGQPPKLLIYWASTRESG | |
| | VKGRFTISRDNAKNSLYLQMN | | VPDRFSGSGSGTDFTLTISSL | |
| | SLRAEDTALYYCAKGGRGSYF | | QAEDVAVYYCQQYYSTPLTFG | |
| | HFDYWGQGTLVTVSS | | GGTQVEIK | |
| AB-001515 | EVQLVESGGGLVQPGGSLRVS | 413 | QSALTQPRSVSGSPGQSVTIS | 874 |
| | CAASGFTFSNYWMIWVRQPPG | | CTGTSIDVGGYNYVSWYQQHP | |
| | KGLVWVSRITSDGSSTAYADS | | GKAPKVMIYDVSKRPSGVPDR | |
| | VKGRFTISRDNAKNTLYLQMN | | FSGSKSGNTASLTISGLQAED | |
| | SLRAEDTAVYYCARGSTSYFG | | EADYYCCSYAGTFTFSVVFGG | |
| | SGSTNYWGQGTLVTVSS | | GTKLTVL | |
| AB-001518 | EAQLLESGGGLVQPGESLRLS | 414 | DIQMTQSPSSLSASVGDRVTI | 875 |
| | CAASRFILSRHGLSWVRQAPG | | TCQASQHINNYLNWYQQKPGK | |
| | KGLEWVSAISGSGVTTFYADS | | APKLLIYHASNLETGVPSRFS | |
| | VKGRFTVSRDNSINRLYLQMD | | GSGSGTHFTFTISNLQPEDIA | |
| | SLRADDTAIYYCARDMSASGW | | TYYCQQFDSLPPPTFGQGTKL | |
| | AIFDSWGQGILVTVSS | | DLK | |
| AB-001519 | QEQLQQWGAGLLKPSETLSLT | 415 | DIQLTQSPSFLSASVGDRVSI | 876 |
| | CAVYGGSFSNYYWTWIRQPPG | | ACRASQGISTYLAWYQQMPGK | |
| | KGLEWIGQIDHSGGTNYSPSL | | APKLLIHSASTLQSGVPSRFS | |
| | KSRVSILVDTSKSQFSLQLTS | | GSGSGTEFTLTITNLQPGDFA | |
| | VTAADTAVYFCARGFIQRHED | | TYYCQQLNTYPRGFGQGTKVE | |
| | YFLCSANRCPQSVFFDLWGRG | | IR | |
| | TLVAVSS | | | |
| AB-001520 | EAQLVESGGGLVQPGGSLTLS | 416 | EVVLTQSPGTLSLSPGERATL | 877 |
| | CAASRLGIDGEWMSWVRQAPG | | SCRASQTVTSNFVSWFQQRRG | |
| | KGLEWVANIRWDRSQIFYADF | | QSPRLLIYGTSSRAAGIPDRF | |
| | VKGRFAISRDDAKKSLFLQMT | | SGSGSGTDFTLTINRLEPEDF | |
| | GLRAEDTALYYCARKNSFDYW | | AVYYCQQYGSFPITFGPGTKV | |
| | GRGTQVTVSS | | DIR | |
| AB-001521 | EVQVVESGGGLVKPGGSLRLS | 417 | EVVMTQSPDTLSVSPGERVTL | 878 |
| | CTASELSFATYSLNWVRQAPG | | SCRASQSVRSNLAWYQQRRGQ | |
| | KGLEWVSSISSSGIRIYYGDP | | APRLLIYGASTRATGIPARFS | |
| | VKGRFTISRDNAKNSLFLQMN | | GSGSGTEFTLTIDSLQSEDFA | |
| | DLRVEDTAVYFCAIRKSCSGI | | IYYCQQYNNWPPWTFGQGTTV | |
| | CPYDYWGQGTLVTVSS | | EIK | |
| AB-001523 | QVQLVESGGGVVQPGRSLRLS | 418 | DIQMTQSPSSLSASVGDRVTI | 879 |
| | CVASGFSLRSFGMHWLRQAPG | | TCRASQDIRNELGWYQQKPGM | |
| | KGLEWVAVSWFDGSDQRYADA | | APKRLIYDASTLQIGVPSRFS | |
| | VKGRFTISRDNSKNTLYLQMD | | GSGSGTEFTLTISSLQPDDFA | |
| | SLRAADTGVYHCGRSLYHNDN | | TYYCLQQNSYPWTFGQGSKVE | |
| | WGQGTLVTVSS | | IK | |
| AB-001526 | QVQLVESGGGVVQPGRSLRLS | 419 | EIVLTQSPATLSLSPGERATL | 880 |
| | CAASGFTFSSYGMHWVRQAPG | | SCRASQGVSRFLAWYQQKPGQ | |
| | KGLEWVAGIWYDGSKKYYGEA | | APRLVIYDVSKRATGIPARES | |
| | VKGRFTISRDDSTDTLYLQMN | | GSGPGPDFTLTISSLEPEDFA | |
| | SLRVEDTAVYYCARDELYSSS | | LYYCQQRSDWPITFGQGTRLE | |
| | PHSNWFDPWGQGTLVTVSS | | IK | |
| AB-001530 | EVQLVESGGGVIPPGGSLRLS | 420 | QSALTQPPSASGSPGQSVTIS | 881 |
| | CAASGFIVNSNYMTWVRQAPG | | CTGTSSDVGGYNYVSWYQHHP | |
| | KGLEWVSVIYSSGNTYYADSV | | GKAPKLMICDVNKRPSGVPDR | |
| | KGRFTISRDNSKNTLFLQMNS | | FSGSKSGNTASLTVSGLQAED | |
| | LRAEDTAVYYCSREPYWGGNQ | | EADYYCSSYAGSNNLVFGGGT | |
| | EDAFDIWGQGTMVTVSS | | KLTVL | |
| AB-001542 | EVQLVESGGGLVKPGGSLRLS | 421 | QTVVTQEPSLTVSPGGTVTLT | 882 |
| | CAASGFIFKNAWMNWVRQAPG | | CASSTGAVTSGYYPNWFQQKP | |
| | KGLEWVGRIRRNSEGATTDYA | | GQAPRPLIYSTSNRHSWTPAR | |
| | APLKGRFTILRDDSKNMLYLQ | | FSGSLLGGKAALTLSGVQPED | |
| | MNSLKTEDTAVYYCTTGVDGH | | EAEYYCLLYCGGPMVFGGGTK | |
| | EHWGQGTLVTVSS | | LTVL | |
| AB-001546 | EVQLVESGGGLVKPGGSLRLS | 422 | SYELTQPPSVSVSPGQTARIT | 883 |
| | CAASGFTFSSYSMNWVRQAPG | | CSGDALPKRYAYWYLQKPGQA | |
| | KGLEWVSSISSSSSYIYYADS | | PVLVIYKDTERPSGIPERFSG | |
| | VKGRFTISRDNAKNSLYLQMN | | SSSGTTVTLTISGVQAEDEAD | |
| | SLRAEDTAVYYCARDLVVAAT | | YYCQSADSSGTHVFFGGGTKL | |
| | DGMDLWGQGTTVTVSS | | TVL | |
| AB-001552 | QVQLVESGGGLVKPGGSLRLS | 423 | SYELTQPPSVSVSPGQTASIT | 884 |
| | CAASGFTFSNAWMNWVRQAPG | | CSGDKLGDKYACWYQQKPGQS | |
| | KGLEWVGRIKSKSVGGTTDYA | | PVLVIYQDSKRPSGIPERFSG | |
| | APVKGRFTISRDDSKNTLYLQ | | SNSGNTATLTISGTQAMDEAD | |
| | MNSLKTEDTAVYYCTTETQLW | | YYCQAWDSSTGVVFGGGTKLT | |
| | FYFDYWGQGTLVTVSS | | VL | |
| AB-001553 | QVQLVESGGGVVQPGRSLRLS | 424 | DIQMTQSPFSLSASVGDRVTI | 885 |
| | CAASGFTFSRYGMHWVRQAPG | | SCRAGQNIGTFLNWYQHKPGQ | |
| | KGLEWVALIWHDGINGNYGDS | | APKLLIYSASRLRSGVPSRFS | |
| | VKGRFTISRDNSKSTVYLQMN | | GSGSGTNFALTISSLRPEDLA | |
| | SLRAEDTAVYYCAREDPNIVV | | TYYCQNSDSAPSLTFGGGTRV | |
| | AALDLWGQGTLVTVSS | | EIK | |
| AB-001556 | EVQLVQSGGGLVKPGGSLRLS | 425 | QSALTQPPSASGSPGQSVTIS | 886 |
| | CAASGFTFSIYSMNWVRQTPG | | CTGTSSDVGGYNYVSWYQQHP | |
| | KGLEWVSSISSSSSYIYYADS | | GKAPKLMIYEVSKRPSGVPDR | |
| | VKGRFTISRDNAKNSLYLQMN | | FSGSKSGNTASLTVSGLQAED | |
| | SLRAEDTAVYYCARGSYRFTP | | EADYYCSSHAGSNNLGVFGTG | |
| | GDYWGQGTLVTVSS | | TKVTVL | |
| AB-001562 | EVQLLESGGNLVQPGGSLRLS | 426 | QSALTQPASMSGSPGQSITIS | 887 |
| | CAASGFTFTSYAMSWVRQAPG | | CTGTSSDVGNYNYVSWYQQHP | |
| | KGLEWVSVISGSGGSTYYVES | | GKAPKLMIYDVSNRPSGVSNR | |
| | VKGRFTISRDNSKSTVYLQMN | | FSGSKSGNTASLTISGLQAED | |
| | SLRAEDTAVYYCARRGPVGYY | | EADYYCTSYTSISTYLFGTGT | |
| | FDYWGQGTLVTVSS | | RVTVL | |
| AB-001564 | EVQLVESGGGLVKPGGSLRLS | 427 | QSVLTQPPSASGTPGQRVTIS | 888 |
| | CAASGFTFSSYSMSWVRQAPG | | CSGSSSNIGSNFVYWYQQLPG | |
| | KGLEWVSSISSSSTYIYYADS | | TAPKLLIYRNNQRPSGVPDRF | |
| | VKGRFTISRDNAKNSLYLQMN | | SGSTSGTSASLAISGLRSEDE | |
| | SLRAEDTAVYYCARDPVWSGF | | ADYYCAAWDDSLSGWVFGGGT | |
| | LFDYWGQGTLVTVSS | | KLTVL | |
| AB-001567 | EVQLVESGGGLVKPGGSLRLS | 428 | EIVLTQSPATLSLSPGERATL | 889 |
| | CAASGFTFSSYSMNWVRQAPG | | SCRASQSVSSYLAWYQQKPGQ | |
| | KGLEWVSSISSSSSYIYYADS | | APRLLIYDASNRATGIPARFS | |
| | VKGRFTISRDNAKNSLYLQMN | | GSGSGTDFTLTISSLEPEDFA | |
| | SLRAEDTAVYYCARTKLWTGS | | VYYCQQRSNWPLTFGQGTRLE | |
| | GMDVWGQGTTVTVSS | | IK | |
| AB-001569 | QVQLVESGGGVVQPGRSLRLS | 429 | DIQMTQSPSSLSASVGDRVTI | 890 |
| | CAASGFTFSIYAMHWVRQAPG | | TCRASQGISNYLAWFQQKPGK | |
| | KGLEWVAVIWYDGSNKYYADS | | APKSLIYAASSLQSGVPSKES | |
| | VKGRFTISRDNSKNTLYLQMN | | GSGSGTDFTLTISSLQPEDFA | |
| | SLRAEDTAVYYCASATGYSSG | | TYYCQQYNSYPLTFGGGTKVE | |
| | YPDYWGQGTLVTVSS | | IK | |
| AB-001573 | QVQLVESGGGVVQPGRSLRLS | 430 | DIQMTQSPSSVSASVGDRVTI | 891 |
| | CAASGFIFSSYGMHWVRQAPG | | TCRASQGISSWLAWYQQKPGK | |
| | KGLEWVAVVWYDGNNKYYADS | | APKLLIYAASSLQSGVPSRFS | |
| | VKGRFTISRDNSKNTLYLQMN | | GSGSGTDFTLTISSLQPEDFA | |
| | SLRADDTAVYYCARDPYFLTG | | TYYCQQANSFPHTFGQGTKVE | |
| | YHGYFDYWGQGTLVTVSS | | IK | |
| AB-001575 | QVQLVQSGAEVKKPGASVKVS | 431 | DIQMTQSPSSLSASVGDRVTI | 892 |
| | CKASGYTFTSYAMHWVRQAPG | | TCRASRSISSSLNWYQQKPGK | |
| | QRLEWMGWINAGNGNTKYSQK | | APKLLIYAASSLQSGVPSRFS | |
| | FQGRVTITRDTSASTAYMELS | | GSGSGTDFTLTISSLQPEDFA | |
| | SLRSEDTAVYYCARLRGDHCT | | TYYCQQSYNTPLFTFGPGTKV | |
| | NGVCPSPHYYYYYYMDVWGKG | | DIK | |
| | TTVTVSS | | | |
| AB-001576 | QVQLVESGGGVVQPGGSLRLS | 432 | NFMLTQPHSVSESPGKTVTIS | 893 |
| | CAASGFTLNSYGIQWVRQAPG | | CTRSGGRIDNNYVQWYQQRPG | |
| | KGLEWVAFIRYDGSKRYYADS | | SAPTMVIYEDNQRPSGVPDRF | |
| | VKGRFTISRDNSKNTLYLQMN | | SGSIDSSSNSASLTISGLKTE | |
| | SLRAEDTAVYYCAKDKRGQNY | | DEADYYCQSYDSDFWVFGGGT | |
| | GMDVWGQGTTVTVSS | | KLTVL | |
| AB-000285 | QVQLQESGPGLVKPSETLSLT | 433 | QAVVTQEPSLTVSPGGTVTLT | 894 |
| | CSVSGGSVSSGSFYWSWIRQP | | CGSSTGAVTSGHYPYWFQQKP | |
| | PGKGLELIGYISYRGYTSYNP | | GQAPTTLIYDTNNKASWTPAR | |
| | SLNSRATISVDTSKNSFSLKL | | FSGSLLGDKAALTLSGAQPDD | |
| | NSVTAADTAVYYCARTGASPG | | EADYYCLLCHNGARDWVFGGG | |
| | YWGQGTLVTVSS | | TKLTVL | |
| AB-000300 | EVQLVESGGALVQPGGSLRLS | 434 | QAVVTQEPSVTVSPGGTVILT | 895 |
| | CAASGFTFSNYWMNWVRQAPG | | CGSSTGAVTSGHYPYWFQQKP | |
| | KGLEWVANIKPDGSQKYYVDS | | GQAPRTLIYDTNNKYSWTPAR | |
| | VKGRFTISRDNAKNSVYLQMN | | FSGSLLGGKAALTLSGAQPED | |
| | SLRVDDSAIYYCIPRDYKWGQ | | EAEYYCLFWYSYVGLIGGGTR | |
| | GILVTVSS | | LTVL | |
| AB-000305 | QLLLQQSGPGLVKPSETLSLT | 435 | QSVLTQPPSVSGAPGQRVTIS | 896 |
| | CSVSAGSNIRSSYYWGWIRQP | | CTGSSSNIGANYNVHWYQQFP | |
| | PGKGLEWIGSTYWSGLAYYNP | | GTAPKLLIYDNVNRPSGVPDR | |
| | SLKRRVTISVDTSKNHFSLKL | | FSGSKSGTSASLAITGLRVED | |
| | SSVTAADTAVYYCARQAYSSG | | ESDYYCQSYDSSLSGWVFGGG | |
| | WAAFGHYYFMDVWGKGTTVTV | | TKLTVL | |
| | SS | | | |
| AB-000312 | EVQLVDSGGGLVKPGGSLRLS | 436 | SYELTQPPSVSVSPGQTASIT | 897 |
| | CAASGFTFSTYTMNWVRQAPG | | CSGDKLGNKYACWYQQKPGQS | |
| | KGLEWVSSITSSGNYIYYADS | | PLLVIYEDNKRPSGIPERFSG | |
| | LKGRFTISRDNAKNSVYLQMN | | SNSGNTATLTISGTQAMDEAD | |
| | SLRGEDTAVYYCARVSGMAAA | | YYCQAWDNRLWVFGGGTKLTV | |
| | ENYYMDVWGKGTTVTVSS | | L | |
| AB-000350 | QVQLVESGGGVVQPGRSLRLS | 437 | DIVLTQSPLSLPVTPGEPASI | 898 |
| | CAASGFTFSNYGMHWVRQAPG | | SCRSSQSLLHSNGYNYLNWYL | |
| | KGLEWVALIWFDGSNKHYADS | | QKPGQSPQLLIYLASNRASGV | |
| | LQGRFTVSRDNSKNTLYLQMN | | PDRFSGSGSGTHFTLKISRVE | |
| | SLRPDDTAIYYCAKSIGRVVI | | AEDVGVYYCMQARLSYTFGQG | |
| | SDLEVAFDYWGRGTLVTVSS | | TKLSIK | |
| AB-000368 | DVQLLESGGGLVQPGGSLRLS | 438 | DIQMTQSPSSLSASVGDRVTI | 899 |
| | CAASGFTFSRYAVNWVRQAPG | | TCRASQGIRNHLAWYQQKPGK | |
| | KGLEWVSFISGPGVSTYYADS | | VPKLLIYAASTLQSGVPSRFS | |
| | VKGRFTISRDNSKNTLYLQMN | | GSGSGTDFTLTISSLQPEDVA | |
| | NLRAEDTAVYYCAKVGPFPST | | TYYCQKCHSAPYTFGQGTKVE | |
| | YYDFWSDDYGLDVWGQGTTVT | | IK | |
| | VSS | | | |
| AB-001398 | EVQLLESGGGLVQPGGSLTLS | 439 | DILMTQSPSSLSASVGDRVTI | 900 |
| | CAGSGFTFKGYAMSWVRQAPG | | TCQASHDINNYLNWYQQKPGK | |
| | KGLEWVSAISGGGDKTYYADS | | APKLIYDASNLETGVASRFSG | |
| | VKGRITISRDNSKNTLYLRLD | | SGSGTEFTLTIRSLQPEDFAT | |
| | SLRAEDTANYYCAKSIQREVV | | YYCQQYESLPITFGQGTRLEI | |
| | IFRLHYWGQGTPVTVSS | | K | |
| AB-001400 | QVQLVQSGAEVKKPGSSVKVS | 440 | DIQMTQSPSSLSASVGDRVTI | 901 |
| | CKASGGTSSTYAFSWVRQAPG | | TCRASQGISNYLAWFQQKPGK | |
| | QGLEWMGGIIPVFGTTNYAQK | | APKSLIYAASSLQSGVPSKES | |
| | FQGRVTITAGESMTAAYMELS | | GSGSGTDFTLTISSLQPEDFA | |
| | SLRSEDTAVYYCASRVVVVAL | | TYYCQQYNSYPYTFGQGTRLE | |
| | NYYYGMDVWGQGTTVTVSS | | IK | |
| AB-001408 | QVQLQESGPGLVKPSETLSLT | 441 | EIVMTQSPATLSVSPGERATL | 902 |
| | CAVSGYSISSGYYWGWIRQPP | | SCRASQSVSSNLAWYQQKPGQ | |
| | GKGLEWIGSIYHSGSTYYNPS | | APRLLIYGASTRATGIPARFS | |
| | LKSRVTISVDTSKNQFSLKLS | | GSGSGTEFTLTISSLQSEDFA | |
| | SVTAADTAVYYCARHAGYSYA | | VYYCQQYNNWPPLFTFGPGTK | |
| | GRGYNWFDPWGQGTLVTVSS | | VDIK | |
| AB-001434 | QVQLVESGGGVVQPGRSLRLS | 442 | DIVMTQTPLSSPVTLGQPASI | 903 |
| | CAASGFTFSSYGMHWVRQAPG | | SCRSSQSLVHSDGNTYLSWLQ | |
| | KGLEWVAVIWYDGSNKYYADS | | QRPGQPPRLLIYKISNRFSGV | |
| | VKGRFTISRDNSKNTLYLQMN | | PDRFSGSGAGTDFTLKISRVE | |
| | SLRAEDTAVYYCARVDLTEWL | | AEDVGVYYCMQATQSWTFGQG | |
| | VLSYWGQGTLVTVSS | | TKVEIK | |
| AB-001440 | EVQLLESGGGLVQPGGSLRLS | 443 | QPVLTQPPSASASLGASVTLT | 904 |
| | CAASGFTFSSYAMSWVRQAPG | | CTLSGGYSNYKVDWYQQRPGK | |
| | KGLEWVSAISGSGGSTYYADS | | GPRFVMRVGTGGIVGSKGDGI | |
| | VKGRFTISRDNSKNTLYLQMN | | PDRFSVLGSGLNRYLTIKNIQ | |
| | SLRAEDTAVYYCAKVSSDDFW | | EEDESDYHCGADHGSGSNFVW | |
| | SGYYQDFDYWGQGTLVTVSS | | VFGGGTKLTVL | |
| AB-001448 | EVQLVESGGGLVKPGGSLRLS | 444 | SYELTQPPSVSVSPGQTASIT | 905 |
| | CAASGFTFSNAWMSWVRQAPG | | CSGDKLGDKYACWYQQKPGQS | |
| | KGLEWVGRIKSKTDGGTTDYA | | PVLLIYQDSKRPSGIPERFSG | |
| | APVKGRFTISRDDSKNTLYLQ | | SNSGNTATLTISGTQAMDEAD | |
| | MNSLKAEDTAVYFCTTDLAQL | | YYCQAWDSSTAVFGGGTKLTV | |
| | WSNFDYWGQGTLVTVSS | | L | |
| AB-001450 | QVQLVQSGSELKKPGASVKLS | 445 | DIVMTQSPDSLAVSLGERATI | 906 |
| | CKASGYTFTTYAINWVRQAPG | | NCKSSQSVLYSSNNKNYLAWY | |
| | QGLEWMGWINTNTGNPMYAQG | | QQKPGQPPKLLIYWASTRESG | |
| | FTGRFVFSLDTSITTAYLQIS | | VPDRFSGSGSGTDFTLTISSL | |
| | SLKAEDTAVYSCARLGQTKNA | | QAEDVAVYYCQQYYSTPFTFG | |
| | FDIWGQGTMVTVSS | | PGTKVDIK | |
| AB-001451 | ERQLEESGGGLVMPGRSLRLS | 446 | QSALTQPASMSGSPGQSITIS | 907 |
| | CVASGFTVGDVYMSWVRQAPG | | CTLTDEHHFVSWYQQHPGKVP | |
| | KGLEWVGRIKSKTDGATTDYT | | KLLIYDAIHRSSGESGRESAS | |
| | PSLRGRFIITRDESKNTLDLQ | | QSGNTASLTISGLRFEDEADY | |
| | LNSLNIEDTGVYYCGSGGHYF | | YCSTYRGDVIFGGGTKLTVL | |
| | DPWGQGTLVTVSS | | | |
| AB-001452 | EEQLVEAGGGLVKPGGSLRLS | 447 | AIQLTQSPSSLSASVGDRVTI | 908 |
| | CAASGFTFSYYSMNWVRQAPG | | TCRASQDISSALAWYQQKPGK | |
| | KGLEWVSSVSPRSDHIYYADS | | PPKVLIFDASTLESGVPSRFS | |
| | VRGRFTISRDNAKNSLYLQMN | | GSGSGTDFTLTITSLQPEDFA | |
| | SLRAEDTAIYYCVRDERGEYD | | AYYCQQFNSYPRTFGQGTRLE | |
| | YLRGIYVYLPDYWGQGTLVTV | | IK | |
| | SS | | | |
| AB-001460 | EMQLVESGGGLVQPGGSLRLS | 448 | EIVLTQSPGTLSLSPGERATL | 909 |
| | CAASGFTFSTYWMSWVRQAPG | | SCRASQSVSSDLAWYQQKPGQ | |
| | KGLEWVANIQQDGIEQYFVDF | | APRLVIYDAFNRATGIPARES | |
| | VEGRFTISRDNAKNSLYLQMN | | GSGSGTDFTLTISRLEPEDFA | |
| | SLRAEDTAVYFCARRGANSHN | | VYYCQQRNKWPLTFGGGTKVE | |
| | GAFDIWGQGTLVTVSS | | IK | |
| AB-001470 | EVQLVQSGAEMKKPGESLKIS | 449 | DIQMTQSPSSLSASVGDRVTI | 910 |
| | CQGSGYKFTNYWIGWVRQMPG | | TCRASQSISRYLNWYQQKPGK | |
| | KGLEWMGIIFPGDSDTRYSPS | | APKLMIFAISTLQSGVPSRFS | |
| | FQGQVTISVDKSISTAYLQWS | | GSGSGTDFTLTISSLQPEDFA | |
| | SLKASDTAIYYCARQGAHRNS | | TYYCQQSYRTPDTFGQGTKLE | |
| | SGFDYWGQGILVTVSS | | LK | |
| AB-001479 | EVKLLTSGGGLVQPGGSLRLS | 450 | NFKLTQPHSVSGSPGKTVRIS | 911 |
| | CATSGFTFNRYALSWVRQAPG | | CTRSSGNIATNGVQWYQQRPG | |
| | KGLEWVSGISATGAEVYYGDS | | SSPTTLIYEDNERPFGVPDRF | |
| | VRGRFTVSRDNSKNTLYLQMK | | SGSIDTSSNSASLTISGLKTE | |
| | SLRVEDTAVYFCAKDAIAVSR | | DEADYYCQSYEDNYLVFGGGT | |
| | NLYYFDYWGLGTLVTVSS | | KLTVL | |
| AB-001483 | EVQLVESGGGLVKPGGSLRLS | 451 | SYELTQPPSVSVSPGQTASIT | 912 |
| | CAASGFTFSNAWMSWVRQAPG | | CSGDKLGDKYACWYQQKPGQS | |
| | KGLEWVGRIKSKTDGGTTDYT | | PVLVIYQDTKRPSGIPERFSG | |
| | APVKGRFTISRDDSKNTLYLQ | | SNSGNTATLTISGTQAMDEAD | |
| | MNSLKTEDTAVYYCTTEPLED | | YYCQAWDSSTAVFGGGTQLTV | |
| | WYPRFDPWGQGTLVTVSS | | L | |
| AB-001484 | EVQLVESGGGLVQPGGSLRLS | 452 | ETVLTQSPATLSLSPGERATL | 913 |
| | CAASEVTFRGYWMHWVRQAPG | | SCRASQSVRSNLAWYQHKPGQ | |
| | KGLVWVSRIYTDGSSTNYADS | | APRLLIYDASNRATGIPARFS | |
| | VKGRFTISRDNAKNTLYLQMN | | GSGSGTDFTLTISSLEPEDFA | |
| | SLRVEDTAVYYCAREANSGWY | | VYYCQQRNNWPPGEVTFGGGT | |
| | GVGQYYFDYWGQGTLVTVSS | | KVEIK | |
| AB-001524 | EVQLVESGGGLVQPGGSLRLS | 453 | GIVLTQSPGTLSLSPGDRATL | 914 |
| | CTTSGFIFSTYWMTWVRQAPG | | SCRASQTENSAYLAWYQQKPG | |
| | KGLEWVATIQENGNEKYYVDS | | QPPRLLIYSASARASGTPDRF | |
| | VKGRFTISRDNAKNSLYLQMN | | SGSESGTVFTLTISRLAPEDF | |
| | SLRAEDTALYYCVSVGEYRTA | | AVYYCHQYHLSPFTFGPGTKV | |
| | SDYWGQGTLVTVSS | | EIN | |
| AB-001527 | QVQLQQWGAGLLKPSETLSLT | 454 | SSELTQDPAVSAALGQTVRIT | 915 |
| | CAVYGGSFSSYYWGWIRQPPG | | CRGDSLRKYYASWYQLKPGQA | |
| | KGLEWIGDINHSGRTNYNTSF | | PVLVIYRKNSRPSGIPDRFSG | |
| | KGRVAVSVDPSKNQFTLKVTS | | SSSGNTASLTITGAQAEDEAD | |
| | VTAADTAVYFCASLEQYDSRW | | YYCNSRDNSGHHLYVFGTGTK | |
| | GWAADYFQHWGQGTLVTVSA | | VTVLS | |
| AB-001539 | EVQLVESGGVVVQPGGSLRLS | 455 | SYVLTQPPSLSVAPGETAWIT | 916 |
| | CAASGFSFDDYTMHWVRHTPG | | CGGNNLGSKSVHWHQQKPGQA | |
| | KGLEWVSLITWDGVTTYYADS | | PVLVIYYDSDRPSGIPERFTG | |
| | VKGRFTISRDNSKNSLYLQMN | | SKSGNTATLTISRVEAGDEAD | |
| | SLRTEDTALYYCAKEIKVAAR | | YYCQVWDRSSDHVVFGGGTKL | |
| | TIDHWGQGTLVTVSS | | TVL | |
| AB-001548 | QVQLVQSGAEVKKPGASVKVS | 456 | DIVMTQSPDSLAVSLGERATI | 917 |
| | CKASGYTFTSYGISWVRQAPG | | NCKSSQSVLYSSNNKKYLAWY | |
| | QGLEWMGWISAYNGNTNYAQK | | QQKPGQPPKLLIYWASTRESG | |
| | LQGRVTMTTDTSTSTAYMELR | | VPDRFSGSGSGTDFTLTISSL | |
| | SLRSDDTAVYYCAREGLLWER | | QAEDVAVYYCQQYYSSSYTFG | |
| | DKPPLGLGGFDPWGQGTLVTV | | QGTKLEIK | |
| | SS | | | |
| AB-001549 | QVQLLDSGGGVVQPGRSLRLS | 457 | SSELTQDPAVSVALGQTVRIT | 918 |
| | CAASGFTFSSYAMHWVRQAPG | | CQGDSLRRYYATWYQQKPGQA | |
| | KGLEWVAVISYDGNKKYYTDS | | PVLVIYGKNNRPSGIPDRFSG | |
| | VKGRLTISRDNSKNTVYLQMN | | SSSGNTASLTITGAQAEDEAD | |
| | SLRAEDTAVYYCARDLPVLRY | | YYCNSRDSSANHEVFGGGTKL | |
| | FDWLPERHYGMDVWGQGTTVT | | TVL | |
| | VSS | | | |
| AB-001559 | EVQLVESGGGLVQPGGSLRLS | 458 | DIVVTQSPESLAVSLGERATI | 919 |
| | CEASGFTFSNYAMNWVRQAPG | | NCKSSQSVLYSPNSKNYLAWY | |
| | KGLEWISYIASGSTTKYYADS | | QQRPGQPPKLLIYWASTRESG | |
| | VMGRLTISRDNAKNTLFLELN | | VPERFSGSGSGTDFALTISSL | |
| | SLRAEDTAVYYCARDQDGDYE | | QAEDVGIYYCQQYMIRPFAFG | |
| | VDAFDVWGQGTKVTVSS | | QGTKLEIK | |
| AB-001560 | QVQLVESGGGVVQPGRSLRLS | 459 | SYELTQPPSVSVSPGQTARIT | 920 |
| | CAASGFTFSSLGMHWVRQAPG | | CSGDALPKQYAYWYQQKPGQA | |
| | KGLEWVAVISFDGSNKYYADS | | PMLVIYKDSERPSGIPERFSG | |
| | VKGRFTISRDNSKNTLYLQMN | | SSSGTTVTLTISGVQAEDEAD | |
| | SLRAEDTAVYYCAKLAGATLS | | YYCQSADISGTHVLFGGGTKL | |
| | FDYWGQGTLVTVSS | | TVL | |
| AB-001563 | QVHLVESGGGVVQPGRSLRLS | 460 | EIVMTQSPATLSVSPGERATL | 921 |
| | CVASGFTLSSHAIHWVRQAPG | | SCRASQSVTSNLAWYQQKPGQ | |
| | KGLEWVAIISYDGSQKSYAGS | | APRLLIYGASTRATGIPGRES | |
| | VKGRFTVSRDTSKNTVSLQMN | | GGGSGTEFTLTISSLQSEDFA | |
| | SLTVEDTAVYYCARDGFSGVM | | VYYCQHYNSWPPWTFGQGTKV | |
| | LFGMGYLDYWGQGTLVTVSS | | EIK | |
| AB-001570 | QVQLVQSGAEVKKPGSSVKVS | 461 | EIVLTQSPATLSLSPGERATL | 922 |
| | CKASGGTFSNYAISWVRQAPG | | SCGASQSVSMNYLAWYQQKPG | |
| | QGLEWMGGIIPIFGTANYAQK | | LAPRLLIYDASSRPSGIPDRF | |
| | FQGRVTITADESTSTAYMELS | | SGSGSGTDFTLTISRLEPEDF | |
| | SLRSEDTAVYYCATSLYCIIN | | AVYYCQQYGSSGAFGPGTKVD | |
| | KCSNYYGLDVWGQGTTVTVSS | | IK | |

### Epitopes

In certain embodiments, the anti-CSP antibodies disclosed herein bind to a first epitope of CSP. CSP is composed of an N-terminal domain containing a heparan sulfate binding site for hepatocyte adhesion, a central repeat region, and a structured C-terminal α-thrombospondin repeat (αTSR) that is followed by a GPI anchor, which attaches CSP to the sporozoite membrane. The central repeat region of CSP is highly immunogenic, and in all P. falciparum strains with a CSP sequence available, the repeat region is composed of 1 NPDP (SEQ ID NO: 979) repeat, 3-5 NVDP repeats (SEQ ID NO:1202), and 35-41 NANP repeats (SEQ ID NO:1203) (e.g., a total of Jan. 4, 1938 of NPDP (SEQ ID NO:979)/NVDP (SEQ ID NO:981)/NANP (SEQ ID NO: 924) motifs are present in the P. falciparum 3D7 strain). The repeat region begins with the junctional NPDP (SEQ ID NO: 979) sequence, typically followed by three alternations of NANP (SEQ ID NO: 924) and NVDP (SEQ ID NO:981) sequences, and continues with the remaining NANP (SEQ ID NO: 924) repeats, with most P. falciparum strains having one NVDP (SEQ ID NO:981) interspersed in the middle of the long NANP (SEQ ID NO: 924) repeat region. Pholcharee, T. et al., J. Mol. Bio. 132:1048-1063 (2020).

In certain embodiments, the anti-CSP antibodies disclosed herein bind to the central repeat region of P. falciparum CSP. In certain embodiments, the antibodies disclosed herein bind to P. falciparum CSP protein in the repeat and/or junctional regions that contain NPNA (SEQ ID NO: 923), NPDP (SEQ ID NO:979), and/or NVDP (SEQ ID NO:981) motifs. In certain embodiments, the anti-CSP antibodies disclosed herein bind to the NANP (SEQ ID NO: 924) repeat region of P. falciparum CSP. In certain embodiments, the anti-CSP antibodies disclosed herein bind to a polypeptide comprising the amino acid sequence set forth in SEQ ID NO: 923.

In certain embodiments, the first epitope comprises the amino acid sequence set forth in SEQ ID NOs: 923-974. In certain embodiments, the first epitope consists of the amino acid sequence set forth in SEQ ID NOs: 923-974. SEQ ID NO: 923-974 are provided in Table 4 below.

TABLE 4

| Sequence | SEQ ID | Sequence | SEQ ID |
|---|---|---|---|
| NPNA | 923 | NPNANPNANP | 949 |
| NANP | 924 | PNANPNANPN | 950 |
| ANPN | 925 | NANPNANPNAN | 951 |
| PNAN | 926 | ANPNANPNANP | 952 |
| NANPN | 927 | NPNANPNANPN | 953 |
| ANPNA | 928 | PNANPNANPNA | 954 |
| NPNAN | 929 | NANPNANPNANP | 955 |
| PNANP | 930 | ANPNANPNANPN | 956 |
| NANPNA | 931 | NPNANPNANPNA | 957 |
| ANPNAN | 932 | PNANPNANPNAN | 958 |
| NPNANP | 933 | NANPNANPNANPN | 959 |
| PNANPN | 934 | ANPNANPNANPNA | 960 |
| NANPNAN | 935 | NPNANPNANPNAN | 961 |
| ANPNANP | 936 | PNANPNANPNANP | 962 |
| NPNANPN | 937 | NANPNANPNANPNA | 963 |
| PNANPNA | 938 | ANPNANPNANPNAN | 964 |
| NANPNANP | 939 | NPNANPNANPNANP | 965 |
| ANPNANPN | 940 | PNANPNANPNANPN | 966 |
| NPNANPNA | 941 | NANPNANPNANPNAN | 967 |
| PNANPNAN | 942 | ANPNANPNANPNANP | 968 |
| NANPNANPN | 943 | NPNANPNANPNANPN | 969 |
| ANPNANPNA | 944 | PNANPNANPNANPNA | 970 |
| NPNANPNAN | 945 | NANPNANPNANPNANP | 971 |
| PNANPNANP | 946 | ANPNANPNANPNANPN | 972 |
| NANPNANPNA | 947 | NPNANPNANPNANPNA | 973 |
| ANPNANPNAN | 948 | PNANPNANPNANPNAN | 974 |

In certain embodiments, the anti-CSP antibodies disclosed herein further bind to a second epitope of CSP. In certain embodiments, the second epitope is heterologous to epitopes present in the RTS, S vaccine. The RTS, S vaccine is a pseudo-viral particle vaccine that combines the hepatitis B surface antigen and the central repeat and C-terminal regions of the Plasmodium falciparum (P. falciparum) circumsporozoite protein (CSP). RTS,S consists of two polypeptides; RTS is a single polypeptide chain corresponding to amino acids 207 to 395 of P. falciparum (3D7) that is fused to HBsAg and S is a polypeptide of 226 amino acids that corresponds to HBsAg. Stoute, et. al., N Engl J Med; 336:86-91(1997); RTS,S Clinical Trials Partnership, PLoS Med. 11(7):e1001685, (2014), WO1993/10152.

In certain embodiments, the second epitope comprises a minor repeat or a junctional region of CSP. In certain embodiments, the second epitope comprises a minor repeat and a junctional region of CSP. In certain embodiments, the minor repeat is a DPNA (SEQ ID NO: 977)/NPNV (SEQ ID NO:980)-containing minor repeat. In certain embodiments, the junctional region is a DPNA (SEQ ID NO: 977)/NPNV (SEQ ID NO:980)-containing junctional region. In certain embodiments, the second epitope comprises the amino acid sequence set forth in SEQ ID NOs: 975-1195. In certain embodiments, the second epitope consists of the amino acid sequence set forth in SEQ ID NOs: 975-1195. SEQ ID NO: 975-1195 are provided in Table 5 below.

TABLE 5

| Sequence | SEQ ID | Sequence | SEQ ID |
|---|---|---|---|
| ADGN | 975 | PNANPNVDPNA | 1086 |
| DGNP | 976 | PNVDPNANPNA | 1087 |
| DPNA | 977 | PNVDPNANPNV | 1088 |
| GNPD | 978 | VDPNANPNANP | 1089 |
| NPDP | 979 | VDPNANPNVDP | 1090 |
| NPNV | 980 | ADGNPDPNANPN | 1091 |
| NVDP | 981 | ANPNVDPNANPN | 1092 |
| PADG | 982 | DGNPDPNANPNV | 1093 |
| PDPN | 983 | DPNANPNANPNA | 1094 |
| PNVD | 984 | DPNANPNVDPNA | 1095 |
| VDPN | 985 | GNPDPNANPNVD | 1096 |
| ADGNP | 986 | NANPNVDPNANP | 1097 |
| ANPNV | 987 | NPDPNANPNVDP | 1098 |
| DGNPD | 988 | NPNVDPNANPNA | 1099 |
| DPNAN | 989 | NPNVDPNANPNV | 1100 |
| GNPDP | 990 | NVDPNANPNANP | 1101 |
| NPDPN | 991 | NVDPNANPNVDP | 1102 |
| NPNVD | 992 | PADGNPDPNANP | 1103 |
| NVDPN | 993 | PDPNANPNVDPN | 1104 |
| PADGN | 994 | PNANPNVDPNAN | 1105 |
| PDPNA | 995 | PNVDPNANPNAN | 1106 |
| PNVDP | 996 | PNVDPNANPNVD | 1107 |
| VDPNA | 997 | VDPNANPNANPN | 1108 |
| ADGNPD | 998 | VDPNANPNVDPN | 1109 |
| ANPNVD | 999 | ADGNPDPNANPNV | 1110 |
| DGNPDP | 1000 | ANPNVDPNANPNA | 1111 |
| DPNANP | 1001 | ANPNVDPNANPNV | 1112 |
| GNPDPN | 1002 | DGNPDPNANPNVD | 1113 |
| NANPNV | 1003 | DPNANPNANPNAN | 1114 |
| NPDPNA | 1004 | DPNANPNVDPNAN | 1115 |
| NPNVDP | 1005 | GNPDPNANPNVDP | 1116 |
| NVDPNA | 1006 | NANPNVDPNANPN | 1117 |
| PADGNP | 1007 | NPDPNANPNVDPN | 1118 |
| PDPNAN | 1008 | NPNVDPNANPNAN | 1119 |
| PNVDPN | 1009 | NPNVDPNANPNVD | 1120 |
| VDPNAN | 1010 | NVDPNANPNANPN | 1121 |
| ADGNPDP | 1011 | NVDPNANPNVDPN | 1122 |
| ANPNVDP | 1012 | PADGNPDPNANPN | 1123 |
| DGNPDPN | 1013 | PDPNANPNVDPNA | 1124 |
| DPNANPN | 1014 | PNANPNVDPNANP | 1125 |
| GNPDPNA | 1015 | PNVDPNANPNANP | 1126 |
| NANPNVD | 1016 | PNVDPNANPNVDP | 1127 |
| NPDPNAN | 1017 | VDPNANPNANPNA | 1128 |
| NPNVDPN | 1018 | VDPNANPNVDPNA | 1129 |
| NVDPNAN | 1019 | ADGNPDPNANPNVD | 1130 |
| PADGNPD | 1020 | ANPNVDPNANPNAN | 1131 |
| PDPNANP | 1021 | ANPNVDPNANPNVD | 1132 |
| PNANPNV | 1022 | DGNPDPNANPNVDP | 1133 |
| PNVDPNA | 1023 | DPNANPNANPNANP | 1134 |
| VDPNANP | 1024 | DPNANPNVDPNANP | 1135 |
| ADGNPDPN | 1025 | GNPDPNANPNVDPN | 1136 |
| ANPNVDPN | 1026 | NANPNVDPNANPNA | 1137 |
| DGNPDPNA | 1027 | NANPNVDPNANPNV | 1138 |
| DPNANPNA | 1028 | NPDPNANPNVDPNA | 1139 |
| DPNANPNV | 1029 | NPNVDPNANPNANP | 1140 |
| GNPDPNAN | 1030 | NPNVDPNANPNVDP | 1141 |
| NANPNVDP | 1031 | NVDPNANPNANPNA | 1142 |
| NPDPNANP | 1032 | NVDPNANPNVDPNA | 1143 |
| NPNVDPNA | 1033 | PADGNPDPNANPNV | 1144 |
| NVDPNANP | 1034 | PDPNANPNVDPNAN | 1145 |
| PADGNPDP | 1035 | PNANPNVDPNANPN | 1146 |
| PDPNANPN | 1036 | PNVDPNANPNANPN | 1147 |
| PNANPNVD | 1037 | PNVDPNANPNVDPN | 1148 |
| PNVDPNAN | 1038 | VDPNANPNANPNAN | 1149 |
| VDPNANPN | 1039 | VDPNANPNVDPNAN | 1150 |
| ADGNPDPNA | 1040 | ADGNPDPNANPNVDP | 1151 |
| ANPNVDPNA | 1041 | ANPNVDPNANPNANP | 1152 |
| DGNPDPNAN | 1042 | ANPNVDPNANPNVDP | 1153 |
| DPNANPNAN | 1043 | DGNPDPNANPNVDPN | 1154 |
| DPNANPNVD | 1044 | DPNANPNANPNANPN | 1155 |
| GNPDPNANP | 1045 | DPNANPNVDPNANPN | 1156 |
| NANPNVDPN | 1046 | GNPDPNANPNVDPNA | 1157 |
| NPDPNANPN | 1047 | NANPNVDPNANPNAN | 1158 |
| NPNVDPNAN | 1048 | NANPNVDPNANPNVD | 1159 |
| NVDPNANPN | 1049 | NPDPNANPNVDPNAN | 1160 |
| PADGNPDPN | 1050 | NPNVDPNANPNANPN | 1161 |
| PDPNANPNV | 1051 | NPNVDPNANPNVDPN | 1162 |
| PNANPNVDP | 1052 | NVDPNANPNANPNAN | 1163 |
| PNVDPNANP | 1053 | NVDPNANPNVDPNAN | 1164 |
| VDPNANPNA | 1054 | PADGNPDPNANPNVD | 1165 |
| VDPNANPNV | 1055 | PDPNANPNVDPNANP | 1166 |
| ADGNPDPNAN | 1056 | PNANPNVDPNANPNA | 1167 |
| ANPNVDPNAN | 1057 | PNANPNVDPNANPNV | 1168 |
| DGNPDPNANP | 1058 | PNVDPNANPNANPNA | 1169 |
| DPNANPNANP | 1059 | PNVDPNANPNVDPNA | 1170 |
| DPNANPNVDP | 1060 | VDPNANPNANPNANP | 1171 |
| GNPDPNANPN | 1061 | VDPNANPNVDPNANP | 1172 |
| NANPNVDPNA | 1062 | ADGNPDPNANPNVDPN | 1173 |
| NPDPNANPNV | 1063 | ANPNVDPNANPNANPN | 1174 |
| NPNVDPNANP | 1064 | ANPNVDPNANPNVDPN | 1175 |
| NVDPNANPNA | 1065 | DGNPDPNANPNVDPNA | 1176 |
| NVDPNANPNV | 1066 | DPNANPNANPNANPNA | 1177 |
| PADGNPDPNA | 1067 | DPNANPNVDPNANPNA | 1178 |
| PDPNANPNVD | 1068 | DPNANPNVDPNANPNV | 1179 |
| PNANPNVDPN | 1069 | GNPDPNANPNVDPNAN | 1180 |
| PNVDPNANPN | 1070 | NANPNVDPNANPNANP | 1181 |
| VDPNANPNAN | 1071 | NANPNVDPNANPNVDP | 1182 |
| VDPNANPNVD | 1072 | NPDPNANPNVDPNANP | 1183 |
| ADGNPDPNANP | 1073 | NPNVDPNANPNANPNA | 1184 |
| ANPNVDPNANP | 1074 | NPNVDPNANPNVDPNA | 1185 |
| DGNPDPNANPN | 1075 | NVDPNANPNANPNANP | 1186 |
| DPNANPNANPN | 1076 | NVDPNANPNVDPNANP | 1187 |
| DPNANPNVDPN | 1077 | PADGNPDPNANPNVDP | 1188 |
| GNPDPNANPNV | 1078 | PDPNANPNVDPNANPN | 1189 |
| NANPNVDPNAN | 1079 | PNANPNVDPNANPNAN | 1190 |
| NPDPNANPNVD | 1080 | PNANPNVDPNANPNVD | 1191 |
| NPNVDPNANPN | 1081 | PNVDPNANPNANPNAN | 1192 |
| NVDPNANPNAN | 1082 | PNVDPNANPNVDPNAN | 1193 |
| NVDPNANPNVD | 1083 | VDPNANPNANPNANPN | 1194 |
| PADGNPDPNAN | 1084 | VDPNANPNVDPNANPN | 1195 |
| PDPNANPNVDP | 1085 | | |

In certain embodiments, an anti-CSP antibody disclosed herein specifically binds to a first epitope of CSP and a second epitope of CSP. In certain embodiments, the anti-CSP antibody binds to at least one additional epitope of CSP. In certain embodiments, the first epitope comprises an amino acid sequence set forth in SEQ ID NO. 923-974, and the second epitope comprises an amino acid sequence set forth in SEQ ID NO. 975-1195. In certain embodiments, the first epitope consists of an amino acid sequence set forth in SEQ ID NO. 923-974, and the second epitope consists of an amino acid sequence set forth tin SEQ ID NO. 975-1195. In certain embodiments, the first epitope consists of an amino acid sequence set forth in SEQ ID NO. 923-974, the second epitope consists of an amino acid sequence set forth in SEQ ID NO. 975-1195, and the at least one additional epitope consists of an amino acid sequence set forth in SEQ ID NO. 975-1195.

### Glycosylation of Anti-CSP Antibodies and Variants Thereof

Glycosylation of antibodies and engineered antibodies has been previously disclosed (see, e.g., U.S. Pat. No. 6,602,684, the content of which is incorporated in its entirety). Antibody Fc regions are generally post-translationally modified via the addition of N-glycans at specific asparagine residues on the antibody heavy chain. IgG molecules bear a N-linked glycosylation asparagine of each heavy chain. It has been shown that a modified glycosylation profile can regulate the antibody functions. For example, without any limitation, altered glycosylation can improve the binding affinity or the half-life of the antibody as compared to the non-modified form.

In certain embodiments, the present disclosure provides anti-CSP antibodies and variants thereof with modified glycosylation. In certain embodiments, the antibodies disclosed herein include an Fc region with increased glycosylation. In certain non-limiting embodiments, the Fc region with increased glycosylation includes increased amounts of bisected oligosaccharides. In certain embodiments, the Fc region with increased glycosylation includes increased amounts of nonfucosylated oligosaccharides. In certain embodiments, the Fc region with increased glycosylation includes increased amounts of fucose-containing oligosaccharides.

In certain embodiments, the antibodies disclosed herein include an Fc region with decreased glycosylation. In certain non-limiting embodiments, the Fc region with decreased glycosylation includes reduced amounts of bisected oligosaccharides. In certain embodiments, the Fc region with decreased glycosylation includes reduced amounts of nonfucosylated oligosaccharides. In certain embodiments, the Fc region with increased glycosylation includes reduced amounts of fucose-containing oligosaccharides.

In certain embodiments, the antibodies disclosed herein include a V region with increased glycosylation. In certain non-limiting embodiments, the V region with increased glycosylation includes increased amounts of bisected oligosaccharides. In certain embodiments, the V region with increased glycosylation includes increased amounts of nonfucosylated oligosaccharides. In certain embodiments, the V region with increased glycosylation includes increased amounts of fucose-containing oligosaccharides.

In certain embodiments, the antibodies disclosed herein include a V region with decreased glycosylation. In certain non-limiting embodiments, the V region with decreased glycosylation includes reduced amounts of bisected oligosaccharides. In certain embodiments, the V region with decreased glycosylation includes reduced amounts of nonfucosylated oligosaccharides. In certain embodiments, the V region with increased glycosylation includes reduced amounts of fucose-containing oligosaccharides.

In certain embodiments, the modified glycosylation can be obtained by expressing any of the antibodies disclosed herein in a host cell with altered glycosylation machinery. For example, without any limitation, a host cell can include a functional disruption of the fucosyltransferase gene and antibodies expressed in this host cell with show reduced glycosylation, e.g., reduced fucosylation (see PCT Patent Publication No. WO 99/54342).

In certain embodiments, the present disclosure provides anti-CSP antibody variants disclosed herein including one or more amino acid substitution resulting in the alteration of a glycosylation acceptor site. In certain embodiments, the alteration includes the elimination of the glycosylation acceptor site. In certain embodiments, the alteration includes modification of a glycosylation acceptor site. In certain embodiments, the alteration includes insertion of a glycosylation acceptor site.

As used herein, “glycosylation acceptor site” refers to an amino acid residue of the light chain or heavy chain of the antibody which can be N- or O-glycosylated. In certain embodiments, the N-linked glycosylation acceptor site can be an asparagine residue. In certain embodiments, the O-linked glycosylation acceptor site can be a serine residue, a threonine residue, a tyrosine residue, a hydroxylysine residue, or a hydroxyproline residue.

In certain embodiments, the Fc region of the antibodies disclosed herein includes one or more glycosylation acceptor site. In certain embodiments, the V region of any of the antibodies disclosed herein includes one or more glycosylation acceptor site. In certain embodiments, the light chain of any of the antibodies disclosed herein includes one or more glycosylation acceptor site. In certain embodiments, the heavy chain of any one of the antibodies disclosed herein includes one or more glycosylation acceptor site. In certain embodiments, the light chain variable region of any of the antibodies disclosed herein includes one or more glycosylation acceptor site. In certain embodiments, the heavy chain variable region of any of the antibodies disclosed herein includes one or more glycosylation acceptor site.

### PEGylation and Other Chemical Modifications of Anti-CSP Antibodies and Variants Thereof

The present disclosure provides anti-CSP antibodies and variants thereof including additional modifications. In certain embodiments, the modifications can improve pharmacological properties of the antibodies, e.g., half-life. In certain non-limiting embodiments, the modification includes PEGylation, deamination, derivatization with polymers, lipidation, removal and/or introduction of disulfide bonds, oxidation, and removal of C-terminal lysine

In certain embodiments, the modification is a PEGylation. PEGylation of antibodies and engineered antibodies includes attachment of one or more polyethylene glycol (PEG) to the antibody. In certain non-limitation embodiments, for example, the PEGylation can be carried out by an acylation reaction or an alkylation reaction with a reactive PEG molecule (or an analogous reactive water-soluble polymer). As used herein, the term “polyethylene glycol” refers to any of the forms of PEG that have been used to derivatize other proteins, such as mono (C1-C10) alkoxy- or aryloxy-polyethylene glycol or polyethylene glycol-maleimide.

In certain embodiments, the modification is the derivatization with a hydrophilic polymer. In certain non-limiting embodiments, for example, the hydrophilic polymer can be carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof.

In certain embodiments, the modification is a lipidation. Lipidation is the conjugation of a protein with a lipid. Lipidation of peptides improves metabolic stability, membrane permeability, bioavailability, and changes the pharmacokinetic and pharmacodynamic properties of the peptides. For example, a lipidated peptide has a high affinity with serum albumin resulting in increased half-life and stability. In certain non-limiting embodiments, for example, the lipid can be myristic acid, palmitic acid, stearic acid, lauric acid, cholesterol, and mixtures thereof.

In certain embodiments, the modification is a substitution of an amino acid residue to form a disulfide bond. In certain embodiments, the amino acid substitution introduces a cysteine. Under certain redox conditions, two cysteines can form a non-natural disulfide bond. In certain non-limiting embodiments, the disulfide bond improves the stability of the antibody, e.g., corrected pairing of the antibody chains. In certain embodiments, the cysteine is introduced in the V region. In certain embodiments, the cysteine is introduced in the Fc region. In certain embodiments, the modification is a substitution of an amino acid residue to remove a disulfide bond. In certain embodiments, the amino acid substitution removes a cysteine. In certain embodiments, the cysteine is substituted with a serine. In certain non-limiting embodiments, removing a cysteine improves the stability of the antibody, e.g., improved long-term stability. In certain embodiments, the cysteine is removed in the V region. In certain embodiments, the cysteine is removed in the Fc region.

### Anti-CSP Antibody and Anti-CSP Antibody Variants Conjugates

In certain embodiments, the present disclosure provides an anti-CSP antibody or variant thereof conjugated or linked to therapeutic and/or imaging/detectable moieties. For example, without any limitation, the anti-CSP antibody or variant thereof can be conjugated to a detectable marker, a toxin, or a therapeutic agent. The moiety may be linked to the antibody covalently or by non-covalent linkages.

In certain embodiments, the antibody or variant thereof is conjugated to cytotoxic moiety or other moiety that inhibits cell proliferation. In certain embodiments, the antibody or variant thereof is conjugated to a cytotoxic agent including, but not limited to, a ricin A chain, doxorubicin, daunorubicin, a maytansinoid, taxol, ethidium bromide, mitomycin, etoposide, tenoposide, vincristine, vinblastine, colchicine, dihydroxy anthracin dione, actinomycin, a diphtheria toxin, extotoxin A from Pseudomonas, Pseudomonas exotoxin (PE) A, PE40, abrin, abrin A chain, modeccin A chain, alpha sarcin, gelonin, mitogellin, restrictocin, cobran venom factor, a ribonuclease, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In certain embodiments, the antibody or variant thereof can be linked to an agent such as an enzyme inhibitor, a proliferation inhibitor, a lytic agent, a DNA or RNA synthesis inhibitors, a membrane permeability modifier, a DNA metabolites, a dichloroethyl sulfide derivative, a protein production inhibitor, a ribosome inhibitor, or an inducer of apoptosis.

In certain embodiments, the antibody or variant thereof can be linked to a radionuclide, an iron-related compound, a dye, a fluorescent agent, or an imaging agent. In certain embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nano-compounds; magnetic resonance imaging contrast agents; light absorbing, reflecting and/or scattering agents; colloidal particles; fluorophores, such as near-infrared fluorophores.

In certain embodiments, the present disclosure provides bispecific molecules comprising an anti-CSP antibody, a variant thereof, or a fragment thereof, disclosed herein. The anti-CSP antibody, anti-CSP antibody variant or antigen-binding portions thereof can be derivatized or linked to another functional molecule, e.g., another peptide or protein (e.g., another antibody or ligand for a receptor) to generate a bispecific molecule that binds to at least two different binding sites or target molecules. The anti-CSP antibody or variant thereof disclosed herein can be derivatized or linked to more than one other functional molecule to generate multispecific molecules that bind to more than two different binding sites (e.g., two different epitopes on the CSP protein) and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association, or otherwise) to one or more other binding molecules, such as another antibody, antibody fragment, peptide or binding mimetic, such that a bispecific molecule results. In certain non-limiting embodiments, for example and without any limitation, the bispecific antibody can be created using the knobs-into-holes strategy. This strategy typically involves creation of a first half of a first antibody that recognizes a first antigen, e.g., CSP, and a second half of the antibody that recognizes a second antigen or binding site, and then joining the two halves to create the bispecific antibody. In certain embodiments, the first antigen and the second antigen are different epitopes of the CSP protein.

### Activity

The activity of any of the anti-CSP antibodies disclosed herein can be assessed by using different endpoints. In certain embodiments, the activity is assessed for binding to CSP, either binding to a series of linear peptides with varying lengths representing the immunodominant regions of the CSP protein or to the entire CSP protein. In certain embodiments, the activity is assessed for the ability to protect against challenge with Plasmodium that comprises P. falciparum CSP, e.g., in in vivo animal models of malaria. In certain embodiments, effector function, e.g., ADCC, is also evaluated.

In certain embodiments, the binding activity of an anti-CSP antibody disclosed herein to P. falciparum CSP protein can be assessed by surface plasmon resonance (SPR) using a biosensor system. Systems suitable for use in SPR are, for example, and without any limitation, LSATM (Carterra, Dublin, CA), Biacore™ (General Electric, Boston, MA), and OpenSPR (Nicoya, East Kitchener, ON, Canada). In an exemplary SPR assay, each antibody can be either directly immobilized to a Carterra CMD200M Chip or captured to the CMD200M Carterra Chip with a goat anti-human IgG Fc antibody. The uncoupled antibodies can be washed off and various concentration gradients of the targets can be flowed over the antibodies. In certain experimental conditions, the highest concentration of each target can be in the range of 0.5-8 μg/mL. For better accuracy, each antibody can be immobilized in different locations (e.g., at least 2) on the chip, and the affinity for each antibody-target combination can be determined using multiple (e.g., 4-5) target concentrations according to standard methods. If the variation between the two duplicates is >3-fold, the antibody-target measurement is repeated.

In certain embodiments, the binding activity of an anti-CSP antibody disclosed herein to P. falciparum CSP protein can be assessed by bio-layer interferometry (BLI). For BLI, each of the antigens can be immobilized on sensors according to the manufacturer's instructions. Systems suitable for use in BLI include, but are not limited to, Octet™ (ForteBio, Fremont, CA) and Gator™ (Probelife, Palo Alto, CA). In certain embodiments, for example and without any limitation, the antigen can be biotinylated and immobilized to streptavidin sensors. For better accuracy, each antibody can be evaluated in replicates at a suitable concentration (e.g., 5 μg/mL). If the variation between the two duplicates is >3-fold, the antibody-target measurement is repeated. The assays are typically performed under conditions according to the manufacturer's instructions. The assays can be performed under a temperature in the range of 20° C. to 37° C., for example, 20° C.-25° C. In certain embodiments, the assay is performed at 25° C. In certain embodiments, the assay is performed at 37° C.

In certain embodiments, binding to CSP protein is assessed in a competitive assay format with a reference antibody A. In certain embodiments, a variant anti-CSP antibody disclosed herein can block binding of the reference antibody in a competition assay by about 50% or more.

Anti-CSP antibodies and anti-CSP antibody variants of the present disclosure may also be evaluated in various assays for their ability to mediate FcR-dependent activity.

In certain embodiments, the activity of an anti-CSP antibody can be evaluated in vivo in an animal model, e.g., as described in the Examples section. In certain non-limiting embodiments, for example, the mouse malaria liver burden assay can be used, as disclosed in Flores-Garcia Y, et al. Malar J. 2019; 18(1): 426, doi: 10.1186/s12936-019-3055-9, the content of which is herein incorporated by reference. Mice are administered antibody and infected with transgenic P. berghei expressing GFP-luciferase and P. falciparum CSP protein. Parasite liver load can be evaluated, e.g., by RT-qPCR or by measuring bioluminescence with an IVIS Spectrum imager. A reduction in parasite liver load reflects the prophylactic activity of an antibody.

In certain embodiments, the activity of an anti-CSP antibody can be determined by evaluating the in vivo protection and survival of animal models, e.g., mice. For example, but without any limitation, mice are administered antibody and challenged with transgenic P. berghei expressing P. falciparum CSP protein. The in vivo protection can be determined by detecting blood-stage parasitaemia in microscopy. The survival rate can be determined using the absence of parasitaemia during an observation period, e.g., two weeks, immediately following the challenge. An increased survival rate reflects the prophylactic and/or therapeutic activity of an antibody.

In certain embodiments, an anti-CSP antibody disclosed herein has at least 20%, or at least 30%, or at least 40%, or at least 50%, or at least 60%, or 70%, or greater, of the activity of antibody AB-000317 when evaluated under the same assay conditions. In certain embodiments, an anti-CSP antibody exhibits improved activity, i.e., greater than 100%, activity compared to antibody AB-000317. In certain non-limiting embodiments, an anti-CSP antibody disclosed herein exhibits at least 20%, or at least 30%, or at least 40%, or at least 50%, or at least 60%, or 70%, or greater reduction in parasite liver load as compared to antibody AB-000317. In certain non-limiting embodiments, an anti-CSP antibody disclosed herein exhibits at least 20%, or at least 30%, or at least 40%, or at least 50%, or at least 60%, or 70%, or greater increase in survival rate as compared to antibody AB-000317.

In certain embodiments, an anti-CSP antibody variant disclosed herein has at least 50%, or at least 60%, or 70%, or greater, of the activity of AB-000224 when evaluated under the same assay conditions. In certain embodiments, an anti-CSP antibody exhibits improved activity, i.e., greater than 100%, activity compared to AB-000224. In certain embodiments, the anti-CSP antibody variants disclosed herein have similar activity against malaria infection as compared to AB-000224. In certain embodiment, an anti-CSP antibody variant disclosed herein has at least 50%, or at least 60%, or 70%, or greater, of the activity of AB-007088 when evaluated under the same assay conditions. In certain embodiments, an anti-CSP antibody exhibits improved activity, i.e., greater than 100%, activity compared to AB-007088. In certain embodiments, the anti-CSP antibody variants disclosed herein have similar activity against malaria infection as compared to AB-007088. The term “similar activity,” when used to compare in vivo activity of antibodies, refers to that two measurements of the activity is no more than 30%, no more than 25%, no more than 20%, no more than 15% different, no more than 10%, no more than 8%, or no more than 5% different from each other.

### Generation of Antibodies

CSP antibodies and variants thereof disclosed herein can be produced using vectors and recombinant methodology (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors.

The present disclosure provides isolated nucleic acids encoding a VH and/or VL region, or fragment thereof, of any of the anti-CSP antibodies and anti-CSP antibody variants disclosed herein. In certain embodiments, the present disclosure provides vectors comprising said nucleic acids and host cells into which the nucleic acids are introduced that are used to replicate the antibody-encoding nucleic acids and/or to express the antibodies. These nucleic acids can encode an amino acid sequence containing the VL, and/or an amino acid sequence containing the VH of the anti-CSP antibody or variant thereof (e.g., the light and/or heavy chains of the antibody). In certain embodiments, the host cell contains (1) a vector containing a polynucleotide that encodes the VL amino acid sequence and a polynucleotide that encodes the VH amino acid sequence, or (2) a first vector containing a polynucleotide that encodes the VL amino acid sequence and a second vector containing a polynucleotide that encodes the VH amino acid sequence.

In certain embodiments, the present disclosure provides a method of making an anti-CSP antibody disclosed herein. In certain embodiments, the method includes culturing a host cell previously described under conditions suitable for expression of the antibody. In certain embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected can vary according to the host cell intended to be used, useful cloning vectors generally can self-replicate, can possess a single target for a particular restriction endonuclease, and/or may carry genes for a marker that can be used in selecting clones containing the vector. Non-limiting examples include plasmids and bacterial viruses, e.g., pUC18, pUC19, Bluescript (e.g., pBS SK+) and its derivatives, mp18, mp19, pBR322, pMB9, ColE1 plasmids, pCR1, RP4, phage DNAs, and shuttle vectors.

Expression vectors generally are replicable polynucleotide constructs that contain a nucleic acid of the present disclosure. The expression vector can replicate in the host cells either as episomes or as an integral part of the chromosomal DNA. Suitable expression vectors include, but are not limited to, plasmids and viral vectors, including adenoviruses, adeno-associated viruses, retroviruses, and any other vector.

Suitable host cells for expressing an anti-CSP antibody or anti-CSP antibody variant disclosed herein include both prokaryotic or eukaryotic cells. For example, but without any limitation, anti-CSP antibodies can be produced in bacteria, in particular when glycosylation and Fc effector function are not needed. After expression, the antibody can be isolated from the bacterial cell lysate in a soluble fraction and can be further purified. Alternatively, the host cell can be a eukaryotic host cell, including, without limitation, eukaryotic microorganisms, such as filamentous fungi or yeast, fungi and yeast strains whose glycosylation pathways have been “humanized,” resulting in the production of an antibody with a partially or fully human glycosylation pattern, vertebrate, invertebrate, and plant cells. Non-limiting examples of invertebrate cells include insect cells. Numerous baculoviral strains have been identified which may be used in conjunction with insect cells. Plant cell cultures can also be utilized as host cells. In certain embodiments, vertebrate host cells are used for producing anti-CSP

antibodies of the present disclosure. For example, without any limitation, mammalian cell lines that can be used to express anti-CSP antibodies include monkey kidney CV1 line transformed by SV40 (COS-7); human embryonic kidney line (293 or 293 cells); baby hamster kidney cells (BHK); mouse Sertoli cells (TM4 cells); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells; MRC 5 cells; and FS4 cells. In certain embodiments, the mammalian cell line used to express anti-CSP antibodies can be Chinese hamster ovary (CHO) cell line; DHFR-CHO cell line (Urlaub et al., Proc. Natl. Acad. Sci. USA 77:4216, 1980); and myeloma cell lines such as YO, NSO, and Sp2/0. Host cells of the present disclosure also include, without limitation, isolated cells, in vitro cultured cells, and ex vivo cultured cells.

A host cell transfected with an expression vector encoding an anti-CSP antibody of the present disclosure, or fragment thereof, can be cultured under appropriate conditions to allow expression of the polypeptide to occur. The polypeptides can be secreted and isolated from a mixture of cells and medium containing the polypeptides. Alternatively, the polypeptide can be retained in the cytoplasm or a membrane fraction and the cells harvested, lysed, and the polypeptide isolated using a desired method.

### Pharmaceutical Compositions and Methods of Treatment

In certain embodiments, the present disclosure provides pharmaceutical compositions for the administration of an anti-CSP antibody and variants thereof. In certain embodiments, the pharmaceutical compositions can be administered to a mammalian subject, e.g., a human, who has malaria or is at risk for malaria, in a therapeutically effective amount and according to a schedule sufficient to prevent Plasmodium infection, e.g., infection with Plasmodium falciparum or a Plasmodium sp. having a cross-reactive CSP protein, or to reduce a symptom of malaria in the subject. In certain embodiments, the pharmaceutical compositions can include any of the anti-CSP antibodies and variants thereof disclosed herein, or a polynucleotide encoding the same, and a pharmaceutically acceptable diluent or carrier. In certain embodiments, a polynucleotide encoding the antibody can be contained in a plasmid vector for delivery, or a viral vector. In certain embodiments, the pharmaceutical composition comprises a therapeutically effective amount of the antibody. As used herein, a “therapeutically effective dose” or a “therapeutically effective amount” refers to an amount sufficient to prevent, cure, or at least partially arrest malaria or symptoms of malaria. A therapeutically effective dose can be determined by monitoring a patient's response to therapy. Typical benchmarks indicative of a therapeutically effective dose include amelioration or prevention of symptoms of malaria in the patient, including, for example, and without limitation, reduction in the number of parasites. Amounts effective for this use will depend upon the severity of the disease and the general state of the patient's health, including other factors such as age, weight, gender, administration route, etc. Single or multiple administrations of the antibody will be dependent on the dosage and frequency as required and tolerated by the patient.

In certain embodiments, the antibody is administered at a pre-erythrocyte stage of infection, i.e., the antibody is administered in a time frame to prevent or reduce hepatocyte infection.

Various pharmaceutically acceptable diluents, carriers, and excipients, and techniques for the preparation and use of pharmaceutical compositions are also disclosed herein. Illustrative pharmaceutical compositions and pharmaceutically acceptable diluents, carriers, and excipients are also described in Remington: The Science and Practice of Pharmacy 20th Ed. (Lippincott, Williams & Wilkins 2012). In certain embodiments, each carrier, diluent, or excipient is “acceptable” in the sense of being compatible with the other ingredients of the pharmaceutical composition and not injurious to the subject. Often, the pharmaceutically acceptable carrier is an aqueous pH-buffered solution. In certain non-limiting embodiments, for example, pharmaceutically-acceptable carriers, diluents or excipients include water; buffers, e.g., phosphate-buffered saline; sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In certain embodiments, the pharmaceutical composition can be formulated for any suitable route of administration, including for example, parenteral, intrapulmonary, intranasal, or local administration. Parenteral administration can include intramuscular, intravenous, intraarterial, intraperitoneal, oral, or subcutaneous administration. In certain embodiments, the pharmaceutical composition is formulated for intravenous administration and has a concentration of antibody of 10-100 mg/ml, 10-50 mg/ml, 20 to 40 mg/ml, or about 30 mg/ml. In certain embodiments, the pharmaceutical composition is formulated for subcutaneous injection and has a concentration of antibody of 50-500 mg/ml, 50-250 mg/ml, or 100 to 150 mg/ml, and a viscosity less than 50 cP, less than 30 cP, less than 20 cP, or about 10 cP. In certain embodiments, the pharmaceutical compositions are liquids or solids. In certain embodiments, the pharmaceutical compositions are formulated for parenteral, e.g., intravenous, subcutaneous, intraperitoneal, or intramuscular administration.

In certain embodiments, the formulation of and delivery methods of pharmaceutical compositions are adapted according to the site and the disease to be treated. For example, without any limitation, formulations include those in which the antibody is encapsulated in micelles, liposomes, or drug-release capsules (active agents incorporated within a biocompatible coating designed for slow-release); ingestible formulations; formulations for topical use, such as creams, ointments, and gels; and other formulations such as inhalants, aerosols, and sprays.

In certain non-limiting embodiments, for example for parenteral administration, the antibodies or antigen-binding fragments thereof are formulated in a unit dosage injectable form (solution, suspension, emulsion) in association with a pharmaceutically acceptable, parenteral vehicle. Non-limiting examples of vehicles include water, saline, Ringer's solution, dextrose solution, and 5% human serum albumin. Nonaqueous vehicles such as fixed oils and ethyl oleate may also be used.

The dose and dosage regimen depend upon a variety of factors readily determined by a physician, such as the nature of the infection, the characteristics of the subject, and the subject's history. In certain embodiments, the amount of antibody or antigen-binding fragment thereof administered or provided to the subject is in the range of about 0.1 mg/kg to about 50 mg/kg of the subject's body weight. Depending on the type and severity of the infection, in certain embodiments, about 0.1 mg/kg to about 50 mg/kg body weight (e.g., about 0.1-15 mg/kg/dose) of antibody or antigen-binding fragment thereof may be provided as an initial candidate dosage to the patient, whether, for example, by one or more separate administrations, or by continuous infusion. The progress of the therapy is readily monitored by conventional methods and assays and based on criteria known to the physician or other persons of skill in the art.

An antibody or variant thereof of the present disclosure can be administered to a subject using any route of administration, e.g., systemic, parenterally, locally, in accordance with known methods. Such routes include, but are not limited to, intravenous administration, e.g., as a bolus or by continuous infusion over a period of time, by intramuscular, intraperitoneal, intracerobrospinal, subcutaneous, intraarticular, intrasynovial, intrathecal, oral, topical, or inhalation routes. A subject can be administered an antibody of the present invention one or more times; and can be administered before, after, or concurrently with another therapeutic agent as further described below.

In certain embodiments, the antibodies or variants thereof of the present disclosure can be administered to prevent malaria. In certain embodiments, the antibodies disclosed herein can inhibit or reduce the risk of Plasmodium infection. In certain embodiments, the antibodies disclosed herein can inhibit or reduce the pre-erythrocytic or sporozoite stage of infection. In certain embodiments, the antibodies disclosed herein can prevent malaria by targeting the Plasmodium at an early stage of entry to the vertebrate of a subject, to thereby arrest the infection from taking place.

In certain embodiments, an anti-CSP antibody of the present disclosure can be administered to treat malaria. In certain embodiments, the antibodies disclosed herein can inhibit or reduce the progression of Plasmodium infection in the blood stream. In certain embodiments, the antibodies disclosed herein can inhibit or reduce the risk of transmission of Plasmodium from a subject to another via insect feeding, e.g., mosquito bite or via contact with infected blood.

In certain embodiments, the pharmaceutical compositions disclosed herein can be administered to a pediatric patient. As used herein, the term “pediatric patient” refers to a patient up to the age of 18 years old. In certain embodiments, the pediatric patient is a patient from age 3 months to less than 12 years old. In certain non-limiting embodiments, the pediatric patient can be a patient between from about 1 year old to about 2 years old, from about 2 years old to about 3 years old, from about 3 years old to about 4 years old, from about 4 years old to about 5 years old, from about 5 years old to about 6 years old, from about 6 years old to about 7 years old, from about 7 years old to about 8 years old, from about 8 years old to about 9 years old, from about 9 years old to about 10 years old, or from about 11 years old to about 12 years old. In certain embodiments, the pediatric patient is not responsive or poorly responsive to another treatment to malaria. In certain embodiments, the pediatric patient is human.

In certain embodiments, the dose of the pharmaceutical compositions disclosed herein is administered based on the weight of the pediatric patient. In certain non-limiting embodiments, the dose of the pharmaceutical compositions is about 5 mg/kg, about 10 mg/kg, about 15 mg/kg, about 20 mg/kg about 25 mg/kg, about 50 mg/kg, about 75 mg/kg, about 100 mg/kg, about 150 mg/kg, about 200 mg/kg, about 250 mg/kg, about 300 mg/kg, or about 350 mg/kg. In certain embodiments, the pediatric patient has a weight of from about 2.5 kg to about 5 kg, from about 5 kg to about 10 kg, from about 10 kg to about 15 kg, from about 15 kg to about 20 kg, from about 20 kg to about 30 kg, or from about 30 kg to about 40 kg.

In certain embodiments, the antibody is provided to the subject in combination with one or more additional therapeutic agents used to treat or prevent malaria or a related disease or disorder. In certain embodiments, a method for treating or preventing malaria is provided, comprising administering to the human a therapeutically effective amount of an antibody as disclosed herein, or a pharmaceutically acceptable salt thereof, in combination with a therapeutically effective amount of one or more additional therapeutic agents. In certain embodiments, a method for treating malaria in a human having or at risk of having the infection is provided, comprising administering to the human a therapeutically effective amount of an antibody as disclosed herein, or a pharmaceutically acceptable salt thereof, in combination with a therapeutically effective amount of one or more additional therapeutic agents.

In certain embodiments, when an antibody of the present disclosure as described herein is combined with one or more additional therapeutic agents as described above, the components of the composition are administered as a simultaneous or sequential regimen. When administered sequentially, the combination may be administered in two or more administrations.

In certain embodiments, an antibody as disclosed herein is combined with one or more additional therapeutic agents in a unitary dosage form for simultaneous administration to a patient.

A “patient” refers to any subject receiving the antibody regardless of whether they have malaria. In certain embodiments, a “patient” is a non-human subject, e.g., an animal that is used as a model for evaluating the effects of antibody administration.

“Co-administration” of an antibody disclosed herein with one or more additional therapeutic agents generally refers to simultaneous or sequential administration of an antibody or fragment thereof disclosed herein and one or more additional therapeutic agents, such that therapeutically effective amounts of the antibody or fragment thereof disclosed herein and one or more additional therapeutic agents are both present in the body of the patient. Co-administration includes administration of unit dosages of the antibody disclosed herein before or after administration of unit dosages of one or more additional therapeutic agents, for example, and without limitation, administration of the antibody within seconds, minutes, or hours of the administration of one or more additional therapeutic agents. In certain non-limiting embodiments, for example, a unit dose of an antibody disclosed herein is administered first, followed within seconds or minutes by administration of a unit dose of one or more additional therapeutic agents. In certain non-limiting embodiments, a unit dose of one or more additional therapeutic agents is administered first, followed by administration of a unit dose of an antibody within seconds or minutes. In certain embodiments, a unit dose of an antibody disclosed herein is administered first, followed, after a period of hours (e.g., 1-12 hours), by administration of a unit dose of one or more additional therapeutic agents. In certain embodiments, a unit dose of one or more additional therapeutic agents is administered first, followed, after a period of hours (e.g., 1-12 hours), by administration of a unit dose of the antibody.

The combined administration may be co-administration, using separate pharmaceutical compositions or a single pharmaceutical composition, or consecutive administration in either order, wherein there is optionally a time period while both (or all) therapeutic agents simultaneously exert their biological activities. Such combined therapy may result in a synergistic therapeutic effect. In certain embodiments, it is desirable to combine administration of an antibody of the invention with another antibody directed against another Plasmodium falciparum antigen, or against a different CSP target epitope.

In certain embodiments, the antibody can be administered by gene therapy via a nucleic acid comprising one or more polynucleotides encoding the antibody. In certain embodiments, the polynucleotide encodes an scFv. In certain embodiments, the polynucleotide comprises DNA, cDNA or RNA. In certain embodiments, the polynucleotide is present in a vector, e.g., a viral vector.

### Methods of Selecting Anti-CSP Antibodies as Anti-Malaria Therapeutic Antibodies

Based on the analysis of anti-CSP antibodies as described herein showing that a) antibody protective activity in vivo does not correlate with binding kinetics to the long NANP6 peptide (SEQ ID NO: 1198) (Table 9) but does significantly correlate with k_{off} to CSP and with binding kinetics to both the short NANP (SEQ ID NO: 924)-containing peptide (NPNA3) (SEQ ID NO: 957) and tetrapeptides of minor repeat and junctional region (JR) (Table 9), and that b) avidity afforded by promiscuous binding could drive more protective responses in vivo, provided herein are methods of selecting an anti-CSP antibody for prevention or treatment of malaria (an anti-malaria therapeutic antibody). In certain embodiments, the method comprises analyzing the antibody for binding to a first epitope of the central repeat region of CSP and for binding to a second epitope of CSP that is heterologous to epitopes present in the RTS,S vaccine, wherein the antibody is selected if it binds to both the first epitope of the central repeat region of CSP and the second epitope that is heterologous to epitopes present in the RTS,S vaccine (heterologous epitope). In certain embodiments, the method comprises selecting the antibody as an anti-malaria therapeutic antibody if the antibody binds to a first epitope of the central repeat region of CSP and binds to a second epitope that is heterologous to epitopes present in the RTS,S vaccine (heterologous epitope).

In certain embodiments, the central repeat region of CSP epitope comprises the amino acid sequence NPNA (SEQ ID NO: 923). Epitopes comprising NPNA include, for example, NPNANP (SEQ ID NO: 933), NANPNA (SEQ ID NO: 931), ANPNAN (SEQ ID NO: 932), NANPNANP (SEQ ID NO: 939), ANPNANPN (SEQ ID NO: 940), NPNANPNA (SEQ ID NO: 941), PNANPNAN (SEQ ID NO: 942), (NPNA) 3 (SEQ ID NO: 957) or (NPNA) 4 (SEQ ID NO: 973). In certain embodiments, heterologous epitopes include epitopes of the minor repeat region of CSP, including epitopes comprising DPNA (SEQ ID NO: 977)/NPNV (SEQ ID NO:980) and epitopes of the junctional region of CSP, including epitopes comprising DPNA (SEQ ID NO: 977).

CSP antibodies and variants thereof disclosed herein can be selected as anti-malaria therapeutic antibodies based on their binding specificities. For example, but without any limitation, the CSP antibodies and variants thereof disclosed herein can specifically bind to a first epitope (e.g., one disclosed in Table 4) and a second epitope (e.g., one disclosed in Table 5).

In certain embodiments, the method comprises analyzing the antibody for binding to a first epitope of CSP. In certain embodiments, the first epitope is included in the central repeat region of CSP. In certain embodiments, the first epitope comprises the amino acid sequence set forth in SEQ ID NOs: 923-974. In certain embodiments, the first epitope consists of the amino acid sequence set forth in SEQ ID NOs: 923-974. In certain embodiments, the method also comprises analyzing the antibody for binding to a second epitope of CSP. In certain embodiments, the second epitope is heterologous to epitopes present in the RTS,S vaccine. In certain embodiments, the second epitope comprises the amino acid sequence set forth in SEQ ID NOs: 975-1195. In certain embodiments, the second epitope consists of the amino acid sequence set forth in SEQ ID NOs: 975-1195. In certain embodiments, the antibody is selected as an anti-malaria therapeutic antibody if it binds to both the first epitope and the second epitope.

In certain embodiments, the method further comprises analyzing the antibody for binding to at least one additional epitope of CSP. In certain embodiments, the at least one additional epitope is heterologous to epitopes present in the RTS,S vaccine. In certain embodiments, the at least one additional epitope comprises the amino acid sequence set forth in SEQ ID NOs: 975-1195. In certain embodiments, the at least one additional epitope consists of the amino acid sequence set forth in SEQ ID NOs: 975-1195. In certain embodiments, the antibody is selected as an anti-malaria therapeutic antibody if it binds to the first epitope, the second epitope, and the at least one additional epitope.

In certain embodiments, the antibody binds to the first epitope with a binding affinity (K_{D}) that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M. In certain embodiments, the antibody binds to the second epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M. In certain embodiments, the antibody binds to the first epitope with a K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M; and to the second epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.

In certain embodiments, the antibody further binds to the at least one additional epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M. In certain embodiments, the antibody binds to the first epitope with a K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M; to the second epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M; and to the at least one additional epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.

In certain non-limiting embodiments, the K_{D} can be measured using surface plasmon resonance assays using a BIACORE® at 25° C. with immobilized antigen CM5 chips at ~10 response units (RU). Briefly, after activation of carboxymethylated dextran biosensor chips, each epitope is diluted before injection at a consistent flow rate (e.g., 5 μl/minute). Following the injection of the epitopes, unreacted groups are blocked. Association rates (kₒₙ or kₐ) and dissociation rates (k_{off} or k_{d}) are calculated using binding models that simultaneously fit the association and dissociation sensorgrams. The equilibrium dissociation constant K_{D} is calculated as the ratio kₐ/kₐ (k_{off}/kₒₙ). Additional information on the calculation of the K_{D} can be found in Chen et al., J. Mol. Biol. 293 (1999) 865-881.

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The foregoing written description is considered to be sufficient to enable one skilled in the art to practice the methods and/or obtain the compositions described herein. The following examples and detailed description are offered by way of illustration and not by way of limitation.

The disclosures of all references in the specification are expressly incorporated herein by reference.

### EXAMPLES

The Examples are offered for illustrative purposes only, and are not intended to limit the scope of the present invention in any way. Indeed, various modifications in addition to those shown and described herein will become apparent to those skilled in the art from the foregoing description and fall within the scope of the appended claims.

It is understood that various other embodiments may be practiced, given the general description provided above.

### Example 1. Identification of Functionally Active Anti-CSP Antibodies

### PBs Skewed to Dominant, Mutated Immunoglobulin G Lineages Post-RTS,S Vaccination

The anti-CSP antibodies were discovered in antibody repertoires generated by Immune Repertoire Capture® (IRC®) technology from plasmablast B cells isolated from two donors enrolled in a Phase 2a study evaluating the efficacy of the RTS,S vaccine in preventing malaria infection. The IRC® technology and its use in antibody discovery is well known and disclosed in, e.g., WO 2012148497A2, the entire content of which is herein incorporated by reference. The RTS,S vaccine is a pseudo-viral particle vaccine that combines the hepatitis B surface antigen and the central repeat and C-terminal regions of the CSP protein. RTS,S consists of two polypeptides; RTS is a single polypeptide chain corresponding to amino acids 207 to 395 of P. falciparum (3D7) that is fused to HBsAg and S is a polypeptide of 226 amino acids that corresponds to HBsAg. Stoute, et. al., N Engl J Med; 336:86-91(1997); RTS,S Clinical Trials Partnership, PLoS Med. 11(7):e1001685, (2014), WO1993/10152. The RTS,S vaccine was administered with the adjuvant AS01B to increase efficacy. AS01B is a liposome-based formulation that contains the immunostimulants monophosphoryl lipid A (MPL) and QS21 and was shown to be more immunogenic than another adjuvant, AS02A, used in initial studies. Kester, et al., J Infect Dis 200:337-346 (2009). All study participants were vaccinated with one of two vaccine schedules (standard full-dose: 0, 1, 2 M or fractional-third dose: 0, 1, 7 M), or placebo and subsequently challenged with a controlled human malaria parasite infection (CHMI).

Plasmablasts (PBs) were isolated from PBMCs collected 7 days post-third (P3D; n=22,319 PB) and post-fourth doses (P4D; n=10,429 PB; Table 9) prior to CHMI and were used to generate natively paired heavy and light chain IgG sequences. Almost all (99.2%) of the antibody sequences were divergent from inferred germline precursor sequences. Consistent with previous malaria studies, specific germline heavy and light chain genes and pairings, including IGHV3-30/33, KV1-5, KV3-20, and LV1-40 were observed frequently in the dataset. No significant associations were observed between protection status and multiple IgG sequence and repertoire features examined.

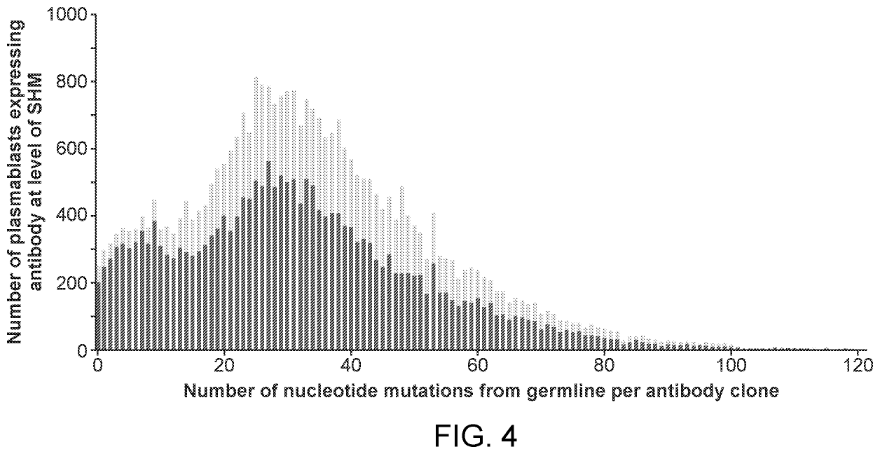

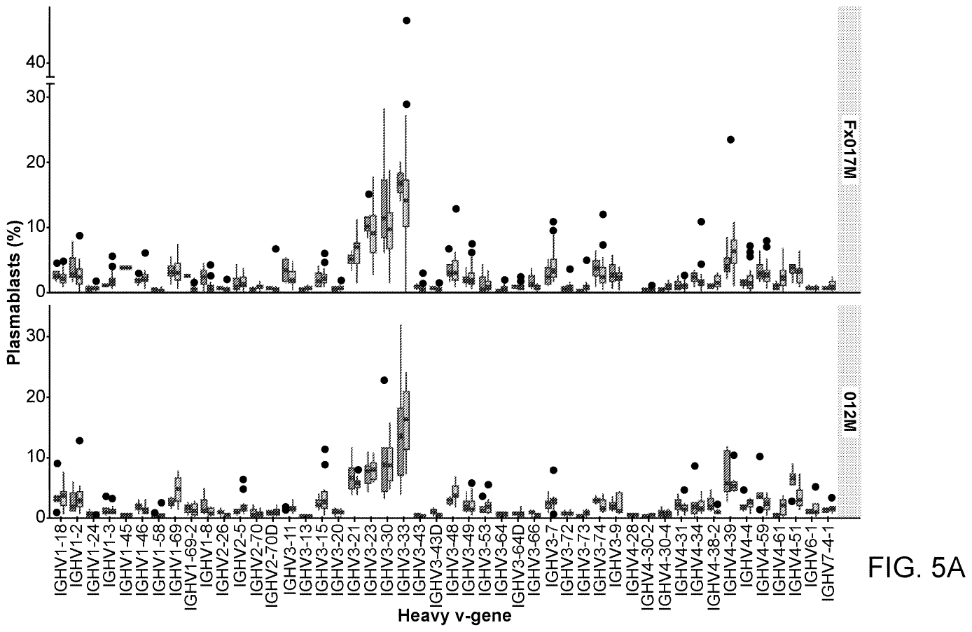

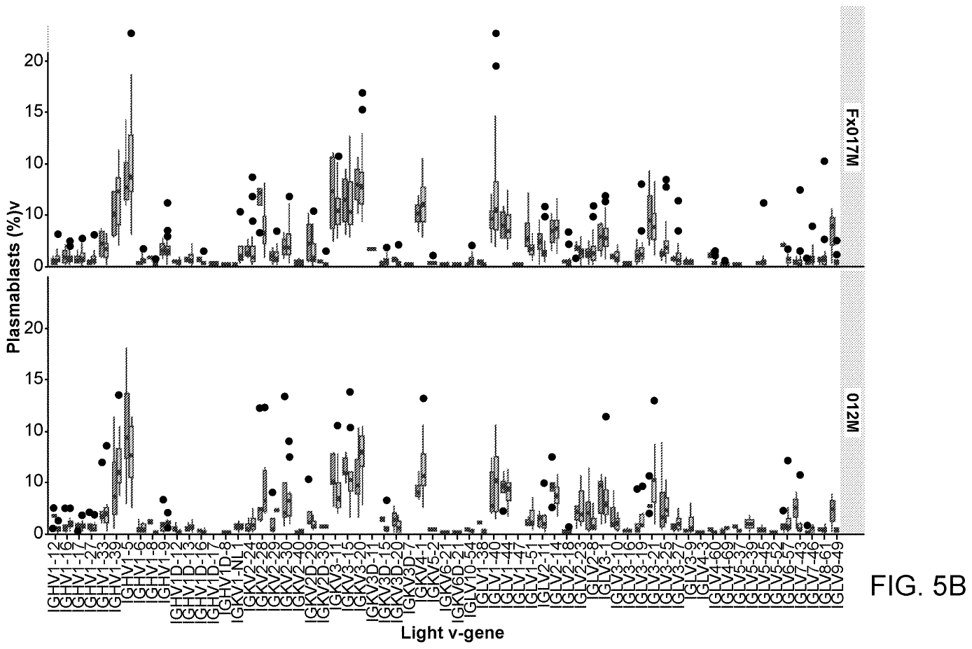

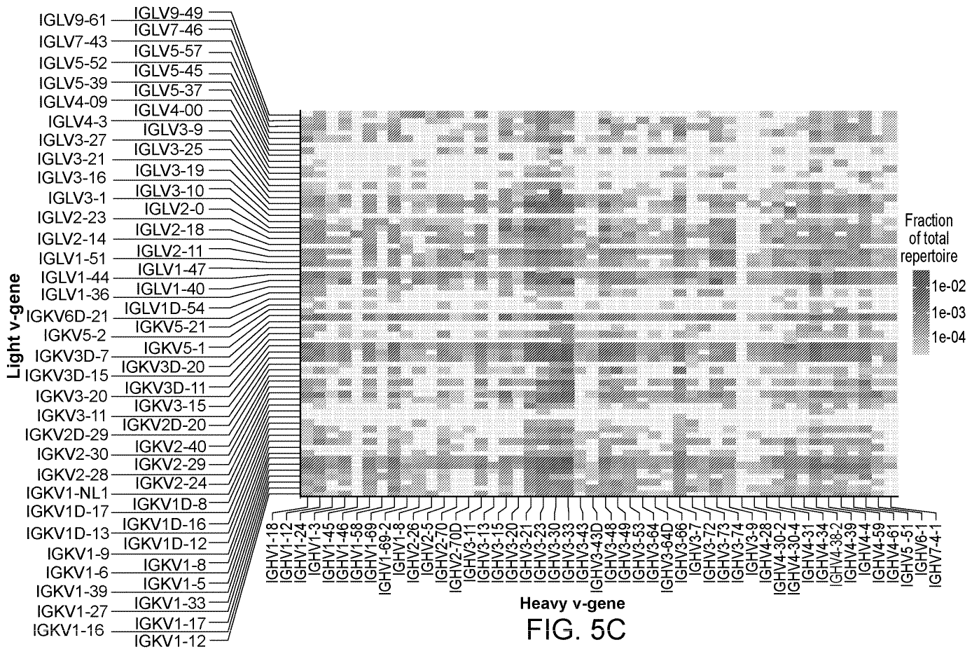

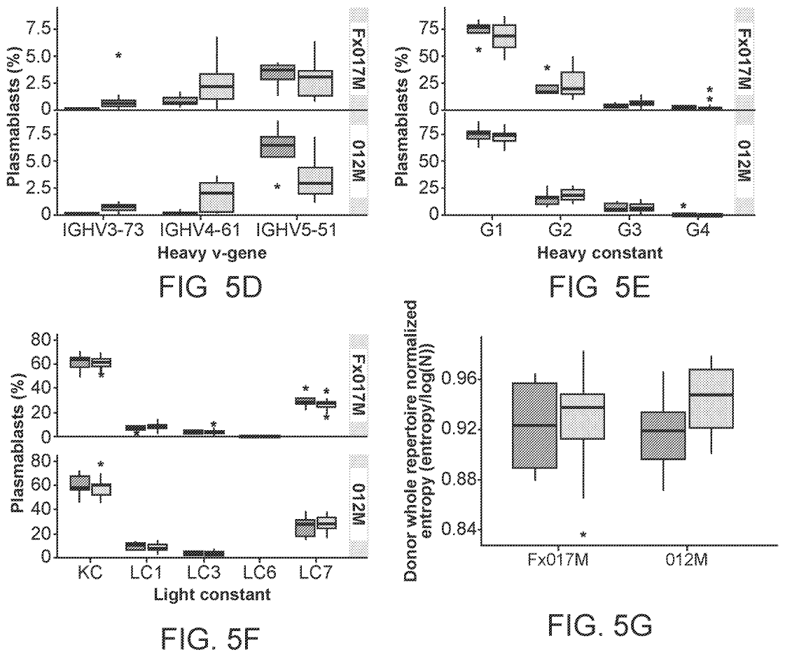

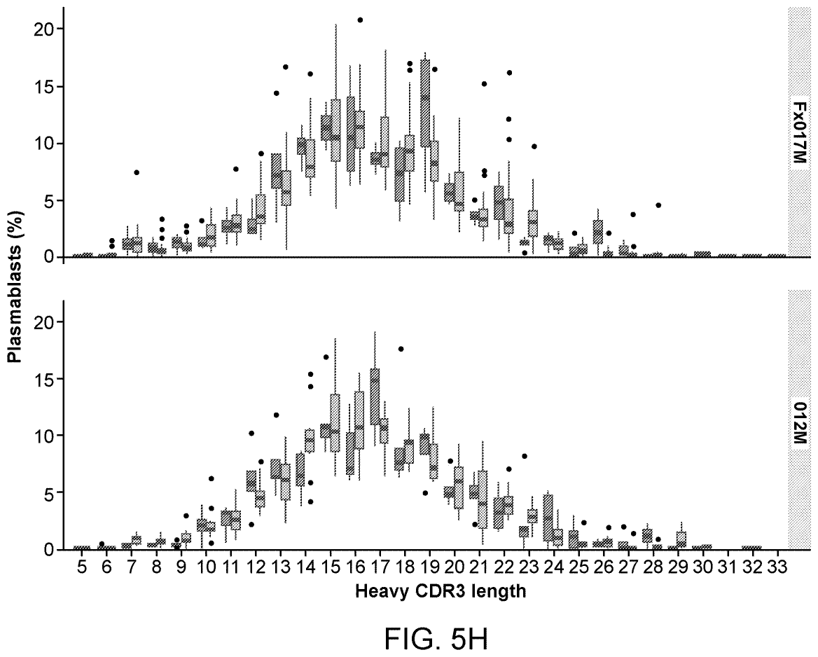

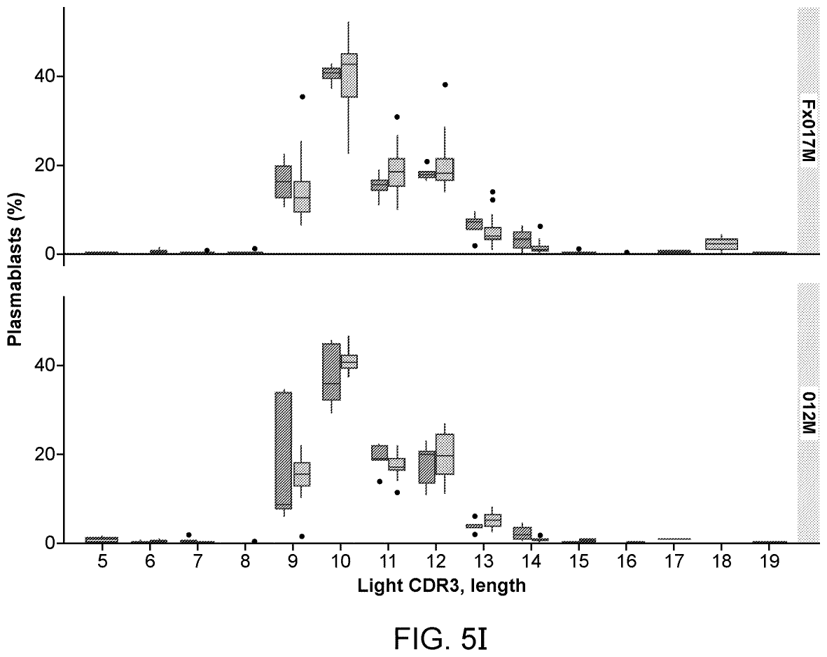

The messenger RNA of immunoglobulin (Ig) G-expressing PBs isolated from peripheral blood mononuclear cells (PBMCs) of individuals (n=45) vaccinated with RTS,S in a phase 2a clinical trial was sequenced using the Immune Repertoire Capture® sequencing platform. In this trial, participants either received three full doses of RTS,S/AS01 E 1 month apart (012M; n=15) or two full doses 1 month apart, followed by a smaller (one fifth, “fractional”) dose 6 months later (Fx017M, n=30). Vaccinees were challenged with malaria in a controlled human malaria infection (CHMI) model after the third dose. A subset received a fourth dose and were challenged a second time with malaria. PBs were isolated from PBMCs collected 7 days post-third (P3D; n=22,319 PB) and post-fourth doses (P4D; n=10,429 PB; Table 6) prior to CHMI and were used to generate natively paired heavy and light chain IgG sequences. Almost all (99.2%) of the antibody sequences were divergent from inferred germline precursor sequences (FIG. 4). Consistent with previous malaria studies, specific germline heavy and light chain genes and pairings, including IGHV3-30/33, KV1-5, KV3-20, and LV1-40 were observed frequently in the dataset (FIGS. 5A-5C). No significant associations were observed between protection status and multiple IgG sequence and repertoire features examined (FIGS. 5D-5I).

TABLE 6
Table 6 | Plasmablasts isolated and sequenced from PBMC of RTS,S vaccinees

| | | Number of PB Sequenced | | P3D sample | | | P4D sample | | | | |
|---|---|---|---|---|---|---|---|---|---|---|---|
| Vaccinee | | P3D | P4D | % PB/ | % PB/ | % B cells/ | % PB/ | % PB/ | % B cells/ | CHMI status | |
| ID | RTS,S dose | Sample | Sample | B cells | PBMC | PBMC | B cells | PBMC | PBMC | P3D | P4D |
| 1 | Fx017M | 263 | NA | 0.61 | 0.034 | 5.6 | NA | NA | NA | P | NA |
| 3 | Fx017M | 672 | 539 | 4.4 | 0.25 | 5.7 | 3.7 | 0.30 | 8.0 | P | P |
| 5 | Fx017M | 620 | 944 | 4.1 | 0.22 | 5.5 | 1.3 | 0.12 | 8.9 | P | P |
| 6 | Fx017M | 520 | NA | 1.0 | 0.056 | 5.3 | NA | NA | NA | P | NA |
| 7 | Fx017M | 374 | 811 | 0.56 | 0.056 | 10 | 0.48 | 0.054 | 11 | P | P |
| 8 | Fx017M | 596 | NA | 0.86 | 0.063 | 7.3 | NA | NA | NA | P | NA |
| 14 | Fx017M | 536 | 735 | 2.1 | 0.16 | 7.7 | 0.73 | 0.071 | 9.7 | P | NP |
| 15 | Fx017M | 529 | NA | 1.5 | 0.15 | 9.7 | NA | NA | NA | P | NA |
| 16 | Fx017M | 136 | 358 | 0.63 | 0.027 | 4.2 | 0.97 | 0.041 | 4.2 | P | P |
| 22 | Fx017M | 476 | NA | 2.0 | 0.24 | 12 | NA | NA | NA | P | NA |
| 23 | Fx017M | 586 | NA | 1.2 | 0.11 | 9.5 | NA | NA | NA | NP | NA |
| 24 | Fx017M | 491 | NA | 0.57 | 0.026 | 4.5 | NA | NA | NA | P | NA |
| 25 | Fx017M | 626 | 5 | 5.9 | 0.27 | 4.7 | 0.69 | 0.044 | 6.4 | NP | P |
| 26 | Fx017M | 516 | NA | 0.85 | 0.078 | 9.2 | NA | NA | NA | P | NA |
| 29 | Fx017M | 574 | NA | 0.69 | 0.053 | 7.7 | NA | NA | NA | P | NA |
| 34 | Fx017M | 735 | 432 | 1.5 | 0.13 | 8.5 | 0.89 | 0.085 | 9.6 | P | P |
| 38 | Fx017M | 397 | NA | 0.54 | 0.048 | 8.9 | NA | NA | NA | P | NA |
| 39 | Fx017M | 693 | NA | 1.8 | 0.084 | 4.7 | NA | NA | NA | P | NA |
| 40 | Fx017M | 685 | NA | 3.1 | 0.17 | 5.6 | NA | NA | NA | P | NA |
| 41 | Fx017M | 606 | 84 | 0.63 | 0.042 | 6.7 | 0.21 | 0.019 | 8.9 | P | P |
| 46 | Fx017M | 474 | 769 | 9.8 | 0.52 | 5.3 | 3.7 | 0.17 | 4.6 | P | P |
| 48 | Fx017M | 583 | NA | 3.7 | 0.091 | 2.5 | NA | NA | NA | P | NA |
| 49 | Fx017M | 569 | NA | 1.4 | 0.11 | 7.8 | NA | NA | NA | NP | NA |
| 51 | Fx017M | 607 | NA | 2.0 | 0.13 | 6.6 | NA | NA | NA | P | NA |
| 52 | Fx017M | 401 | NA | 0.73 | 0.026 | 3.6 | NA | NA | NA | P | NA |
| 53 | Fx017M | 565 | 842 | 0.72 | 0.032 | 4.5 | 0.66 | 0.052 | 7.8 | P | P |
| 54 | Fx017M | 236 | 1027 | 0.84 | 0.034 | 4.1 | 1.1 | 0.072 | 6.4 | NP | P |
| 60 | Fx017M | 632 | NA | 1.3 | 0.061 | 4.8 | NA | NA | NA | P | NA |
| 63 | Fx017M | 206 | NA | 0.71 | 0.015 | 2.1 | NA | NA | NA | P | NA |
| 64 | Fx017M | 940 | 777 | 2.5 | 0.11 | 4.6 | 1.7 | 0.11 | 6.7 | P | P |
| 201 | 012M | 167 | NA | 1.4 | 0.097 | 7.0 | NA | NA | NA | P | NA |
| 202 | 012M | 402 | 653 | 9.0 | 0.43 | 4.7 | 1.1 | 0.066 | 5.8 | NP | NP |
| 203 | 012M | 592 | 923 | 2.4 | 0.13 | 5.4 | 2.3 | 0.14 | 6.3 | P | NP |
| 205 | 012M | 193 | 511 | 1.0 | 0.062 | 5.9 | 0.66 | 0.066 | 10.0 | P | NP |
| 207 | 012M | 469 | 177 | 1.3 | 0.039 | 3.1 | 0.97 | 0.032 | 3.3 | P | P |
| 208 | 012M | 425 | NA | 1.3 | 0.083 | 6.4 | NA | NA | NA | NP | NA |
| 209 | 012M | 324 | NA | 0.92 | 0.063 | 6.9 | NA | NA | NA | P | NA |
| 216 | 012M | 430 | NA | 1.5 | 0.15 | 10 | NA | NA | NA | P | NA |
| 221 | 012M | 385 | NA | 1.1 | 0.13 | 12 | NA | NA | NA | P | NA |
| 228 | 012M | 509 | 477 | 4.9 | 0.27 | 5.6 | 2. | 0.14 | 6.6 | NP | P |
| 229 | 012M | 561 | NA | 1.0 | 0.059 | 5.8 | NA | NA | NA | P | NA |
| 230 | 012M | 482 | 385 | 1.6 | 0.091 | 5.6 | 0.65 | 0.036 | 5.5 | NP | P |
| 233 | 012M | 561 | 180 | 2.2 | 0.16 | 7.2 | 0.52 | 0.031 | 6.0 | P | NP |
| 234 | 012M | 403 | NA | 2.1 | 0.071 | 3.3 | NA | NA | NA | NP | NA |
| 236 | 012M | 572 | NA | 1.4 | 0.090 | 6.7 | NA | NA | NA | P | NA |

NA, not applicable; P, protected; NP, not protected; P3D, post-third dose RTS,S; P4D, post-fourth dose RTS,S

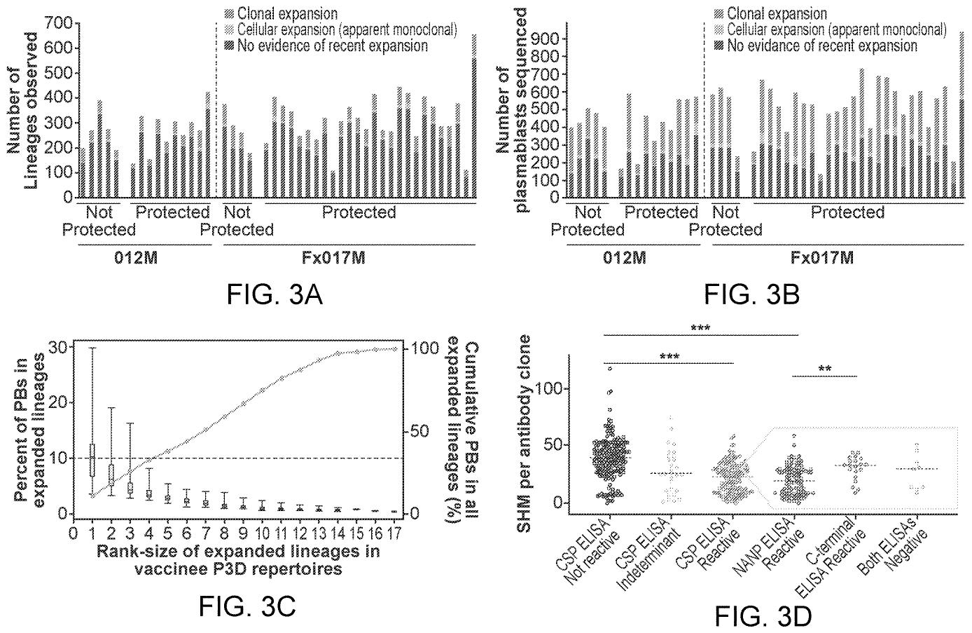

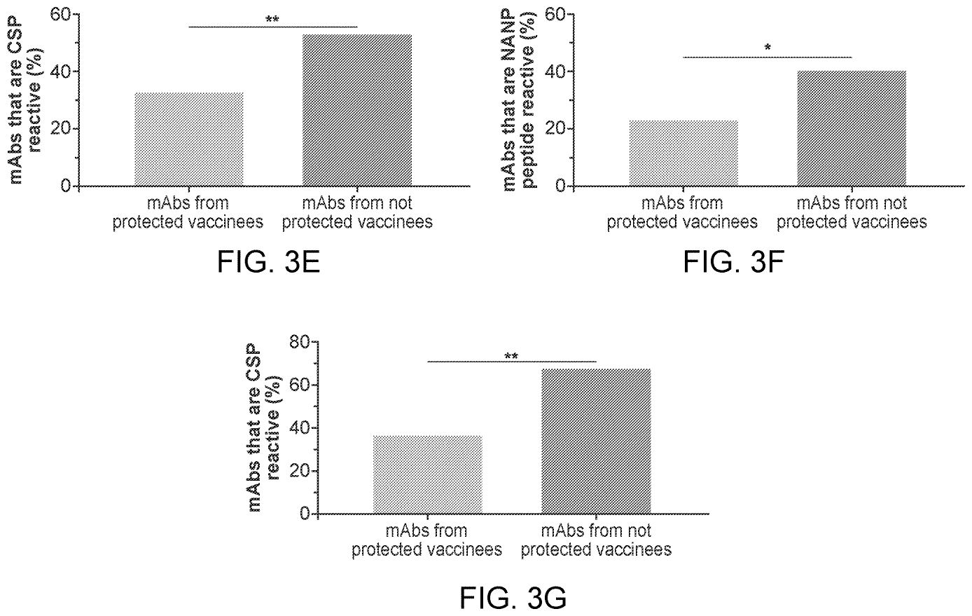

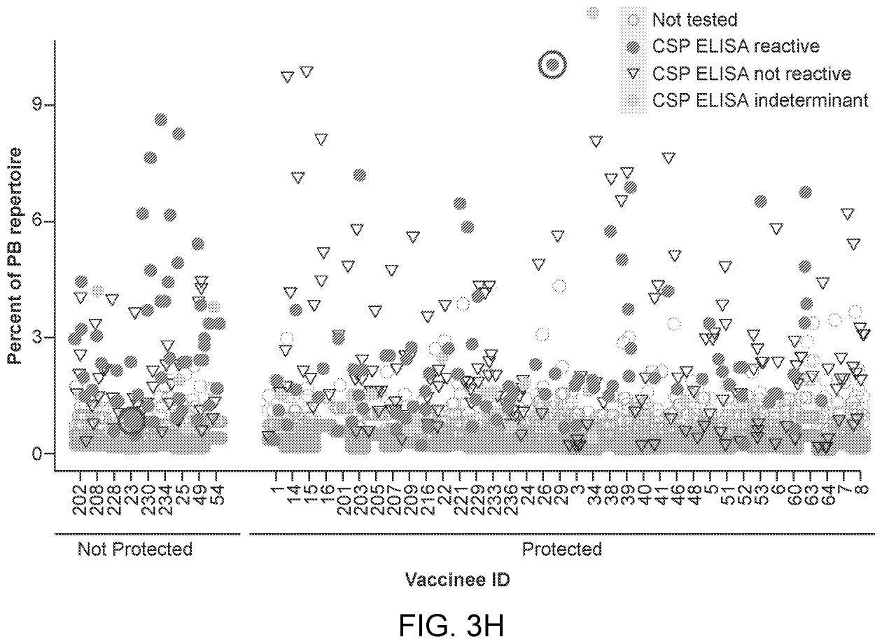

P3D and P4D PBs were grouped into Ig lineages (n=18,980), defined here as PB sequences that were likely derived from a common progenitor B-cell clone (see Example 2). Lineage size ranged from 1-84 (P3D) or 1-93 (P4D) PBs. As PBs have a short half-life in blood and were isolated from a small volume of blood (~10 ml), detection of lineages with ≥2 PBs indicates recent expansion in lymphoid organs. One-fifth of lineages were cellularly expanded and contained at least two PBs with either the same or divergent B-cell nucleotide sequences (19.4%, n=3,684 lineages, FIG. 3A). Consistent with antigen-driven selection pressure following vaccination, most of the cellularly expanded lineages also showed evidence of clonal expansion (FIG. 3B), a hallmark of affinity maturation. Furthermore, several lineages had clonal representatives that were observed after both the third and fourth immunizations, referred to here as “recalled” lineages (4.1%-26.6% of vaccinee P3D expanded lineages). In addition, when sequences were compared between the vaccinees, many of these expanded lineages also show evidence of sequence convergence between ≥2 vaccinees (7.3%-46.7% of vaccinee P3D expanded lineages). Not surprisingly, lineages with only a single observed PB in P3D repertoires (n=10,841) had significantly lower rates of convergence (2.0%-13.8%) and recall (1.2%-18.6%) than the expanded lineages (P<0.0001 and P<0.001, respectively, Wilcoxon matched-pairs, two-tailed test) and had higher levels of somatic hypermutation (SHM). Thus, to increase the chances of identifying antibodies derived against RTS,S antigen, efforts of subsequent analyses were focused on expanded lineages (FIG. 3B).

It was hypothesized that lineages with the largest number of PBs per vaccinee, referred to here as “dominant lineages”, were more likely to target the vaccine, as they had outcompeted other PB lineages for antigen binding and/or T-cell help in lymphoid organs. Thus, for each vaccinee, expanded P3D lineages (9-99 expanded lineages observed per vaccinee) were rank-ordered by size (“rank-size”). The sum of PBs in the lineages of the top four rank-sizes for each vaccinee constituted 17%-100% of the total number of PBs in each vaccinee's P3D repertoire of expanded lineages and 33% of the PBs among the P3D-expanded lineages from all vaccinees (FIG. 3C). Because this pattern of PB distribution was consistent across protection status and dose regimens, an antibody screening library was generated for in vitro and in vivo characterization that was biased toward the dominant P3D lineages of both protected and non-protected vaccinees.

CSP-Reactivity of Expanded P3D PBs is Associated with Lower SHM and Lack of Protection

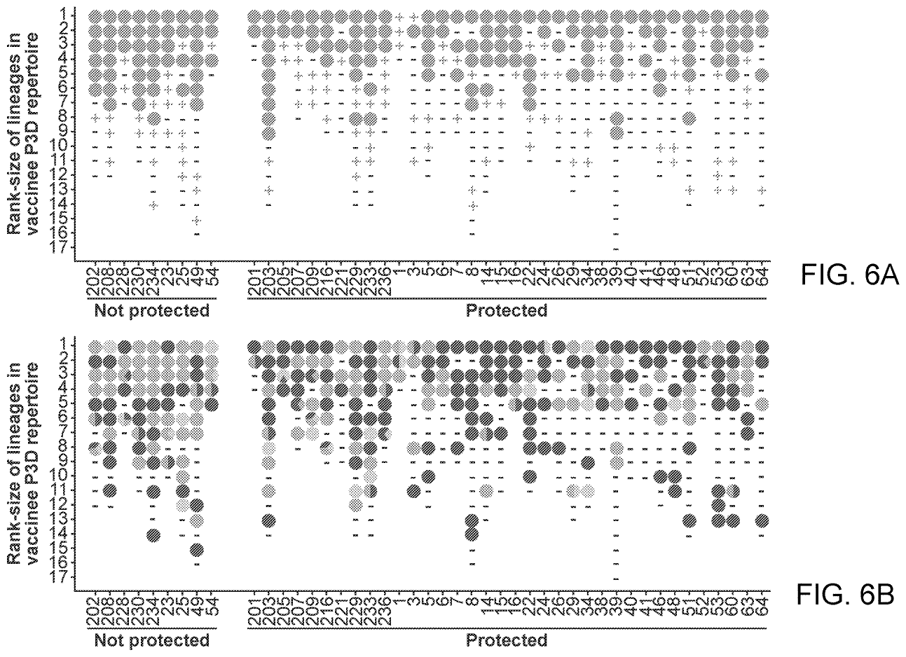

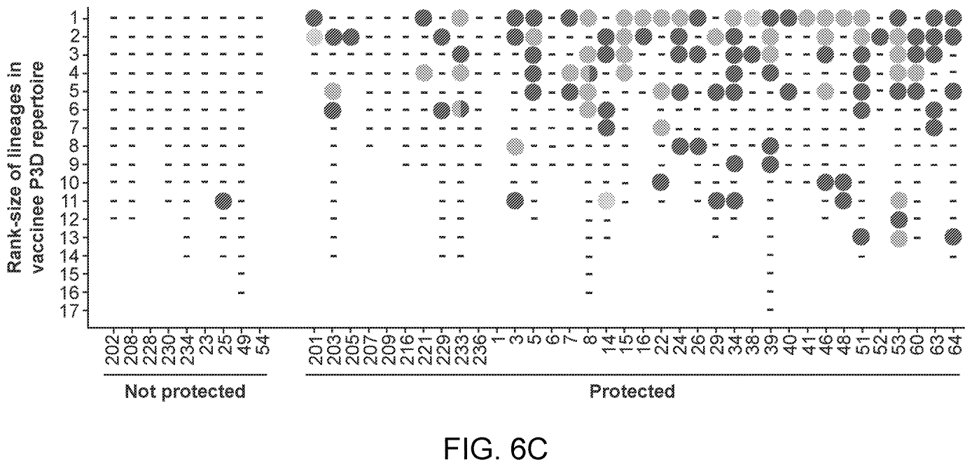

A clone from each of 369 unique P3D lineages was chosen, gene synthesized, and recombinantly expressed for testing (FIG. 6A). This library included almost all (96%) of the largest lineages (rank-size 1) across all vaccinees; approximately half (56%) of the second, third, and fourth rank-size lineages across all vaccinees; a small subset (6.9%) of expanded, sub-dominant lineages (rank-size≥5), and a few single-PB lineages (0.18% of the 10,841 single-cell lineages). All antibodies were screened in a CSP enzyme-linked immunosorbent assay (ELISA) (FIGS. 3D and 6B), and approximately one-third were screened against the other RTS,S component, hepatitis B surface antigen (HBsAg, FIG. 6C). Of the antibodies screened in both assays (n=130), 52% were reactive to CSP or HBsAg (67/130). In total, 38% (139/369) bound to CSP, and binding for an additional 29 antibodies was indeterminate. Of the CSP-reactive antibodies, 73% (102/139) bound peptides from the NANP (SEQ ID NO: 924) CR region and 20 bound peptides from the C-terminal region (data not shown).

Given that expanded lineages were more likely to show evidence of convergence and recall as compared to single PBs, it was tested whether those same features were associated with CSP-reactivity. Indeed, antibodies from lineages that show sequence convergence across ≥2 vaccinees were more likely to bind to CSP (54%, 55/102) than clones from lineages that lacked evidence of convergence (31%, 84/267, P=0.0001, Fisher's exact, two-sided). Recalled lineages were also more likely to be CSP-reactive (49%, 43/87) as compared to lineages only observed P3D (19%, 16/83 P<0.0001, Fisher's exact).

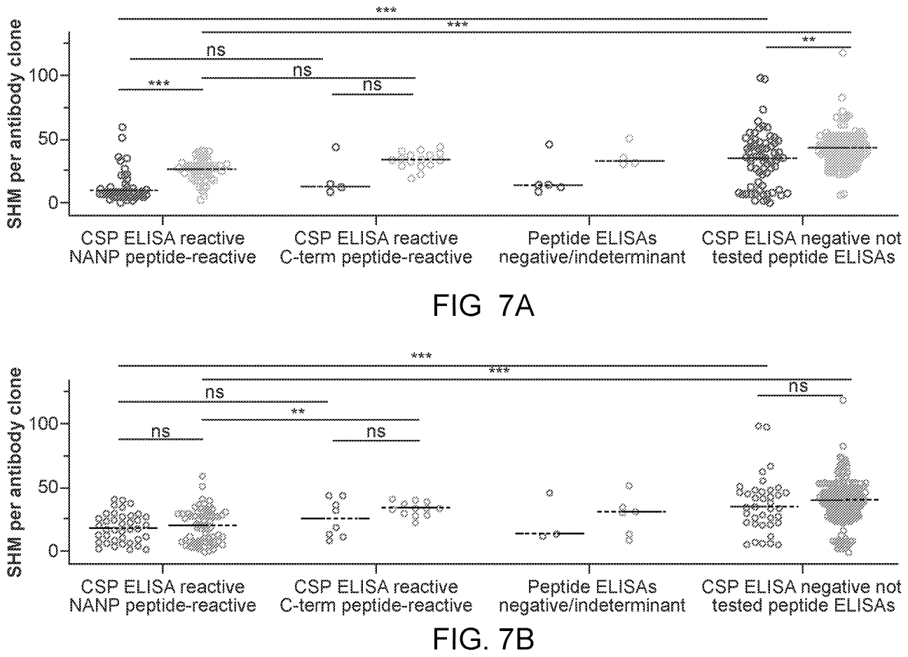

As noted previously for immunization with whole sporozoites, the SHM levels of CSP-reactive antibodies were found to be significantly lower than SHM levels of CSP-negative antibodies (P<0.0001, FIG. 3D), and SHM levels of NANP (SEQ ID NO: 924)-specific antibodies were lower than SHM levels of C-terminal binding antibodies (P<0.006, FIG. 3D). Consistent with observations about these sequence repertoires, SHM levels of NANP-binding antibodies were not correlated with vaccinee protection status P3D (P>0.6, FIG. 7B). Further, the percentage of antibodies that were CSP-specific and NANP (SEQ ID NO: 924)-specific were surprisingly lower among P3D-protected vaccinees than P3D-non-protected vaccinees (P<0.0007 for CSP, P<0.006 for NANP (SEQ ID NO: 924), Fisher's exact, FIGS. 3E and 3F). This inverse correlation between antibody binding and P3D protection status is also observed when the analysis is restricted to just the antibodies from the most dominant lineages (rank-size 1-4, P<0.0004, FIG. 3G), as well as when all antibodies, including the 20 from lineages that have only 1 PB, are combined in the analysis (P<0.0005 Fisher's exact and P<0.00105 by bootstrap analysis, FIG. 3H). These data suggest that the quality of the CSP-specific antibody repertoire may be more important in driving protection than the overall quantity of circulating, CSP- or repeat-specific PBs.

### Sporozoite Inhibitory Antibodies in P3D PBs are not Sufficient for P3D Protection.

Given this surprising inverse association and the well-reported protective activity of CSP-binding antibodies in both humans and mice, antibodies were selected to advance as potential anti-malaria prophylactics without assuming any correlate of protection. Seventy-seven antibodies (77 unique lineages) were selected that included NANP (SEQ ID NO: 924)- and C-terminal-reactive antibodies from protected (n=26) and not protected (n=8) vaccinees, and from dominant and sub-dominant lineages of either high or low SHM levels (respectively, ≥20 or <20 nucleotide mutations from germline per antibody). As in vitro functional assays have demonstrated limited predictive power for in vivo, anti-malaria activity, the antibodies were screened for activity using a mouse sporozoite-infection model. Over half of these antibodies (44/77) provided ≥95% inhibition of sporozoite liver burden, and some offered near-complete protection (≥99.9% inhibition). All 44 antibodies bind to the NANP (SEQ ID NO: 924)-repeat region of CSP, most derived from the IGHV3-33 germline, some from other IGHV3 genes, and one from IGHV1. Thirteen additional NANP (SEQ ID NO: 924) binding, IGHV3-30/33 antibodies demonstrated limited inhibition of parasite liver burden (80%-95%), and 12 antibodies, including three C-terminal peptide binders, showed minimal, but detectable inhibition (20%-80%, FIG. 1A).

Roughly a third of the tested antibodies (30%, 23/77) were from unprotected vaccinees, including half (7/14) that showed near-complete protection in mice (≥99.9% inhibition, FIG. 1A). These data suggest expression of these inhibitory antibodies by circulating, expanded P3D PB lineages is insufficient to drive protection. For example, the highly effective antibody AB-000317 is observed in both a protected vaccinee and a not protected vaccinee (FIG. 3H, red circles). However, PB expansion levels for this antibody lineage differed between the two vaccinees. In the protected vaccinee, the antibody was a member of the largest PB lineage, while in the unprotected vaccinee, the antibody was expressed in the seventh rank-size lineage (respectively, 15.8% versus 1.7% of PB in expanded P3D lineages, and 10% versus 0.9% of all circulating P3D PBs). These data are consistent with the hypothesis that, in addition to the functional activity of an antibody, the number of PBs expressing the antibody may affect protection status by ultimately influencing titer in blood and/or representation in immune memory.

Inhibitory Antibodies from Vaccinees Bind CSP Peptides not Present in RTS,S

To explore the developability of these inhibitory antibodies as potential drugs, 35 NANP-repeat-binding lineages were selected for further pharmacology studies from the 52 that demonstrated ≥90% inhibition in the sporozoite-challenge screen. To avoid sequence features that can potentially act as liabilities during the development of antibody drugs, and to survey clones from lineages that have extensive clonal diversity, more than one unique antibody clone was chosen from some (n=23) lineages. Overall, up to 141 antibodies, from 21 protected vaccinees of both RTS,S dose regimens, representing a range of high and low SHM levels, were tested in binding assays.

Antibodies displayed a broad range of affinities against CSP (K_{D} by surface plasmon resonance [SPR] of 11 pM-9.8 nM, FIG. 1B, Tables 7 and 8). Despite the inverse correlation observed between vaccinee protection status and the percentage of CSP-reactive antibodies observed in the original screening library (FIGS. 3E-3H), these down-selected, inhibitory antibodies had a significant association between CSP-binding affinity (K_{D}) and SHM levels (P<0.005, r=−0.26 and P<0.0001, r=−0.39 for heavy and light chain, respectively, Spearman test), indicating that affinity maturation to CSP occurred following vaccination. These correlations are likely driven by two relationships: binding association rates (kₒₙ) to CSP and heavy and light chain SHM levels (FIGS. 10A and 10B, Table 9) and binding dissociation rates (k_{off}) from CSP and SHM levels in the light chain (P<0.0005, r=−0.29, Spearman; Table 9).

TABLE 7
Table 7 | Binding and in vivo pharmacology of lead mAbs

| | Binding constants (surface plasmon resonance) | | | | | |
|---|---|---|---|---|---|---|
| | K_{D} | | | K_{D} NVDP3NANP2 | K_{D} | K_{D} |
| mAb | CSP | K_{D} NANP6 (SEQ | K_{D} NPNA3 (SEQ | (SEQ ID NO 1200) | NANPNVDPNANP(SEQ | Junction |
| ID | (nM) | ID NO 1198) (nM) | ID NO 957) (nM) | (nM) | ID NO 1097) (nM) | (nM) |
| AB-007104 | 5.74E−02 | 3.66E−02 | 6.17E−01 | 1.16E−01 | 5.67E−01 | 7.11E+03 |
| AB-007088 | 2.15E−01 | 2.36E−01 | 4.68E+00 | 1.97E+00 | NA | 2.52E+02 |
| AB-007103 | 2.10E−02 | 4.42E−02 | 1.70E+00 | 4.38E−01 | 4.13E+02 | NA |
| AB-000224 | 3.66E−02 | 9.94E−02 | 4.53E+00 | 1.68E+00 | 1.42E+03 | NA |
| AB-000399 | 3.25E−01 | 1.35E+00 | 9.79E+02 | 2.23E+02 | 3.00E+03 | NA |
| AB-007114 | 8.67E−02 | 3.13E+00 | 1.37E−01 | 3.24E+00 | 7.85E+03 | NA |
| AB-001529 | 2.28E−01 | 3.13E−01 | 4.75E+02 | 8.09E+02 | NA | NA |
| AB-000364 | 3.03E−02 | 6.37E−01 | 2.55E+00 | 7.72E−01 | 7.12E+00 | 3.29E+03 |
| AB-001480 | 5.18E−01 | 3.18E+00 | 2.23E+02 | 9.87E+00 | 5.05E+03 | 2.16E+04 |
| AB-007176 | 4.94E−02 | 3.88E−02 | 2.61E−01 | 3.97E−02 | 3.89E−01 | 2.37E+03 |
| AB-000317 | 5.44E−02 | 1.29E+00 | 8.04E+00 | 2.75E+03 | 1.44E+04 | NA |
| AB-007131 | 5.35E−02 | 5.93E−01 | 1.37E+00 | 6.41E−01 | 6.97E+00 | NA |
| AB-007101 | 8.39E−01 | 1.17E−01 | 1.84E+00 | 1.16E+00 | 2.51E+01 | 3.13E+04 |
| AB-007161 | 1.19E+00 | 6.49E+00 | 3.39E+03 | 8.23E+01 | 9.45E+03 | 2.07E+04 |
| AB-007121 | 2.59E−02 | 7.57E−02 | 8.97E−01 | 3.74E−01 | 1.32E+00 | 5.43E+02 |
| AB-007173 | 5.31E−02 | 6.03E−01 | 1.11E+02 | 9.35E+00 | 1.94E+04 | 8.38E+03 |
| AB-007154 | 2.67E−02 | 7.87E−01 | 1.65E+01 | 8.99E−01 | 3.12E+00 | 5.49E+03 |
| AB-007083 | 1.41E−01 | 6.59E−02 | 2.08E+00 | 1.26E+00 | 1.91E+00 | 9.40E+02 |
| AB-001558 | 1.05E+00 | 3.23E−01 | 1.74E+03 | 5.32E+00 | 2.03E+03 | 6.82E+03 |
| AB-001387 | 3.74E−01 | 1.87E+00 | 4.01E+03 | 1.24E+01 | NA | NA |
| AB-007462 | 1.07E−02 | 7.71E−02 | 4.36E−02 | 3.22E−01 | 3.69E−01 | 3.17E+01 |
| AB-007087 | 6.53E−02 | 9.86E−02 | 4.43E+00 | 5.15E−01 | 6.37E−01 | 5.05E+02 |
| AB-000397 | 6.46E−02 | 1.26E−01 | 4.34E+01 | 7.79E+03 | 1.54E+04 | NA |
| AB-001428 | 5.87E−01 | 3.80E−01 | 8.34E+02 | 8.30E+00 | 7.62E+03 | 2.44E+04 |
| AB-007178 | 1.20E−01 | 5.33E−01 | 3.42E+00 | 5.47E+00 | 6.37E+01 | 2.95E+04 |
| AB-000391 | 1.16E−01 | 7.60E−02 | 9.63E−01 | NA | NA | NA |
| AB-007120 | 1.66E−01 | 6.91E−01 | 8.68E+01 | 1.02E+01 | 5.27E+03 | 1.48E+04 |
| AB-007110 | 4.20E−02 | 7.26E−02 | 2.13E+00 | 1.22E+00 | 3.14E+02 | 4.19E+04 |
| AB-000334 | 5.75E−01 | 7.49E−01 | 3.15E+01 | 6.58E−01 | 8.50E−01 | 1.66E+02 |
| AB-007096 | 2.04E−01 | 1.33E−01 | 8.22E+00 | 6.59E+00 | 8.41E+02 | NA |
| AB-007123 | 2.17E−01 | 7.57E+00 | 1.55E+00 | 6.10E−01 | 1.12E+00 | 4.15E+02 |
| AB-001472 | 9.77E−01 | 1.56E−01 | 1.38E+03 | 2.01E+03 | NA | NA |
| AB-007182 | 1.58E−01 | 6.45E−01 | 1.10E+03 | 2.81E+00 | 1.70E+03 | 6.05E+04 |
| AB-001554 | 5.15E−01 | 2.31E−01 | 2.24E+02 | 9.49E+02 | NA | NA |
| AB-007130 | 2.01E−01 | 4.47E−02 | 6.63E−01 | 3.25E−01 | 3.34E+00 | 4.04E+03 |
| AB-007168 | 3.98E−02 | 5.87E−01 | 1.91E+00 | 2.68E−01 | 1.29E+00 | 2.49E+02 |
| AB-000356 | 2.44E−01 | 1.11E−01 | 1.93E+00 | 4.59E+00 | 1.31E+03 | NA |
| AB-007150 | 1.88E−02 | 5.11E−02 | 9.63E+00 | 3.38E+02 | 2.98E+02 | 4.65E+03 |
| AB-001455 | 2.86E−01 | 6.15E−02 | 6.94E+00 | 1.36E+01 | NA | NA |
| AB-007175 | 3.28E−02 | 2.81E−02 | 2.16E+00 | 7.92E−01 | 1.23E+01 | 9.71E+02 |
| AB-000373 | 1.71E−01 | 3.16E−02 | 4.09E+00 | 9.34E−01 | 1.94E+01 | 1.12E+04 |
| AB-001533 | 7.50E−02 | 6.13E−02 | 8.86E−01 | 4.34E−01 | 2.54E+00 | 8.77E+02 |
| AB-001458 | 2.51E−01 | 3.19E−01 | 2.09E+03 | 1.02E+02 | 7.19E+01 | NA |
| AB-000231 | 2.25E−02 | 4.14E−02 | 4.38E−01 | 2.87E−01 | 8.97E−01 | 7.77E+00 |
| AB-001396 | 2.34E−01 | 2.79E−01 | 3.18E+01 | 7.09E+00 | 3.38E+02 | 6.79E+04 |
| AB-007109 | 8.36E−02 | 9.10E−02 | 6.39E+00 | 1.54E+00 | 4.17E+02 | NA |
| AB-007090 | 1.56E−01 | 5.10E−02 | 1.72E+00 | 1.73E+00 | 1.23E+02 | 2.74E+04 |
| AB-007080 | 2.27E−01 | 2.55E−02 | 1.62E−01 | 4.84E−01 | 8.42E+00 | 4.31E+03 |
| AB-007102 | 4.19E−02 | 5.85E−02 | 1.50E+02 | 1.25E+01 | NA | NA |
| AB-000376 | 2.62E−01 | 1.92E−01 | 4.48E+02 | 1.44E+01 | 2.25E+04 | 5.29E+04 |
| AB-007076 | 1.24E−01 | 2.64E−02 | 7.10E−02 | 7.07E−02 | 1.20E+00 | 1.92E+03 |
| AB-007106 | 6.53E−01 | 1.91E+00 | 1.44E+02 | 1.06E+01 | 1.27E+03 | NA |
| AB-007107 | 1.16E+00 | 1.32E−01 | 4.87E+02 | 5.39E+02 | NA | NA |
| AB-007111 | 3.29E−02 | 2.28E−01 | 5.08E+00 | 1.85E+00 | 1.37E+03 | NA |
| AB-007159 | 8.86E−02 | 3.27E−01 | 5.26E+02 | 8.69E+02 | 1.79E+04 | NA |
| AB-007170 | 2.41E−02 | 3.13E−02 | 4.79E−01 | 1.64E−01 | 7.86E−01 | 8.41E+03 |
| AB-007165 | 8.00E−02 | 1.88E−01 | 6.71E−01 | 4.19E−02 | 5.97E−01 | 4.21E+02 |
| AB-007115 | 2.17E−01 | 3.61E−01 | 2.15E+01 | 1.02E+01 | 7.36E+01 | 5.74E+03 |
| AB-007138 | 6.67E−02 | 5.32E−01 | 8.61E+00 | 4.62E+01 | 1.06E+02 | 9.62E+02 |
| AB-000327 | 2.95E−02 | 4.68E−02 | 1.04E+01 | 1.97E+00 | 5.36E+02 | 1.15E+04 |
| AB-007160 | 3.78E−01 | 2.74E+00 | 7.73E+02 | 2.35E+03 | 2.18E+04 | NA |
| AB-007112 | 8.84E−02 | 1.78E−01 | 2.15E−01 | 1.54E+00 | 5.41E+02 | NA |
| AB-007142 | 1.33E−01 | 5.62E−02 | 1.06E+01 | 3.05E+02 | 1.43E+04 | NA |
| AB-007126 | 1.03E−01 | 8.98E−02 | 9.81E−01 | NA | NA | NA |
| AB-007113 | 4.37E−02 | 2.11E+00 | 1.37E+01 | 1.32E+00 | 1.17E+03 | NA |
| AB-007108 | 1.02E−01 | 2.49E−01 | 6.14E+01 | 3.23E+00 | 1.01E+04 | NA |
| AB-007118 | 2.92E−02 | 2.64E+00 | 3.18E+01 | 6.37E+01 | 7.49E+01 | 8.40E+03 |
| AB-007158 | 3.27E−01 | 9.93E−02 | 1.09E+02 | 1.08E+02 | 1.48E+03 | 4.13E+03 |
| AB-007169 | 1.75E−02 | 5.29E−02 | 9.73E−01 | 6.18E−01 | 1.47E+00 | 1.27E+03 |
| AB-007139 | 2.97E−02 | 1.33E−01 | 4.52E+00 | 1.33E+02 | 9.94E+01 | 2.53E+03 |
| AB-000394 | 1.10E−01 | 3.94E−02 | 2.78E−01 | 3.01E−01 | 5.05E−01 | 5.09E+03 |
| AB-007075 | 1.06E−01 | 1.31E−01 | 2.27E−01 | 6.19E−01 | 1.60E+00 | NA |
| AB-007077 | 2.36E−01 | 2.67E−01 | 3.26E−01 | 5.91E−01 | 2.41E+00 | NA |
| AB-007078 | 1.59E−01 | 5.19E−02 | 6.86E−01 | 1.73E+00 | 2.35E+02 | 3.50E+04 |
| AB-007079 | 2.25E−01 | 5.70E−02 | 4.51E−01 | 5.88E−01 | 2.47E+01 | NA |
| AB-007081 | 1.50E−01 | 2.80E−02 | 2.51E−01 | 3.74E−02 | 8.38E−01 | 1.10E+04 |
| AB-007082 | 1.35E−01 | 1.88E−02 | 9.90E−01 | 1.45E+00 | 1.54E+01 | 3.63E+04 |
| AB-007084 | 8.00E−02 | 3.43E−01 | 2.29E+00 | 1.23E+00 | 1.36E+00 | 9.44E+01 |
| AB-007085 | 5.04E−02 | 1.23E−01 | 6.44E+00 | 3.10E+00 | 1.89E+01 | 9.37E+02 |
| AB-007086 | 1.61E−01 | 2.24E−01 | 7.08E+00 | 1.22E+00 | 1.04E+01 | 1.26E+02 |
| AB-001545 | 1.71E+00 | 4.36E+01 | 2.50E+03 | NA | NA | NA |
| AB-007089 | 1.55E−01 | 7.38E−01 | 1.04E+01 | 4.46E+00 | 2.14E+03 | NA |
| AB-007091 | 1.88E+00 | 1.43E+00 | 2.26E+03 | 6.02E+02 | NA | NA |
| AB-007092 | 9.75E+00 | 3.61E+01 | 1.16E+04 | NA | NA | NA |
| AB-007093 | 3.67E−01 | 7.75E−02 | 8.21E−01 | 1.81E+00 | 1.52E+02 | 2.56E+04 |
| AB-007094 | 1.36E+00 | 1.44E+00 | 2.81E+02 | 1.69E+02 | NA | NA |
| AB-007095 | 1.50E+00 | 5.99E−01 | 4.04E+01 | 5.04E+01 | NA | NA |
| AB-007097 | 6.54E−01 | 3.88E−01 | 7.22E+01 | 2.74E+01 | 2.04E+03 | NA |
| AB-007098 | 1.55E+00 | 1.37E−01 | 1.56E+01 | 1.45E+02 | NA | NA |
| AB-007099 | 5.55E−01 | 1.14E−01 | 8.85E+00 | 1.00E+01 | 4.41E+03 | NA |
| AB-007100 | 1.72E−01 | 1.38E+00 | 3.95E+02 | 2.20E+02 | NA | NA |
| AB-007184 | 1.37E−01 | 1.30E−01 | 6.99E+00 | 1.21E+00 | 6.88E+02 | NA |
| AB-001511 | 1.23E+00 | 1.97E+00 | 3.89E+02 | 1.05E+02 | NA | NA |
| AB-001578 | 1.00E+00 | 3.06E+00 | 4.38E+02 | 3.55E+02 | 1.97E+04 | NA |
| AB-007105 | 1.49E+00 | 3.97E+00 | 9.57E+01 | 3.26E+02 | NA | 8.89E+04 |
| AB-007116 | 5.78E−01 | 1.39E+01 | 3.13E+03 | 1.16E+03 | 1.02E+04 | 9.39E+03 |
| AB-007117 | 2.77E+00 | 4.21E+01 | 8.34E+03 | NA | NA | NA |
| AB-007119 | 2.23E−01 | 2.06E−02 | 4.60E+00 | 5.05E−01 | 1.43E+01 | NA |
| AB-007122 | 2.23E−02 | 1.10E+00 | 5.33E+00 | 6.85E−01 | 1.51E+01 | 2.18E+03 |
| AB-007124 | 2.78E−02 | 4.68E−01 | 3.40E+01 | 1.36E+00 | 2.75E+02 | 6.37E+03 |
| AB-007125 | 1.31E+00 | 2.05E−01 | 1.42E+03 | NA | NA | NA |
| AB-007127 | 1.03E−01 | 9.62E−02 | 2.33E+00 | NA | NA | NA |
| AB-007128 | 5.66E−02 | 7.99E−01 | 1.19E+00 | NA | NA | NA |
| AB-000325 | 1.39E−01 | 8.60E−02 | 8.85E−01 | 6.05E−01 | 1.12E+01 | NA |
| AB-007129 | 7.48E+00 | 1.26E+01 | NA | NA | NA | NA |
| AB-007132 | 1.72E−01 | 7.33E−02 | 6.94E+00 | 1.78E+00 | 2.11E+02 | NA |
| AB-007133 | 2.18E−01 | 5.67E−01 | 6.35E+02 | 1.02E+00 | 4.04E+02 | NA |
| AB-007134 | 3.21E−01 | 1.50E+00 | 4.92E+03 | 1.30E+02 | NA | NA |
| AB-007136 | 3.50E−01 | 1.61E+00 | 5.02E+02 | 1.97E+03 | NA | NA |
| AB-007137 | 6.23E−01 | 2.13E+00 | 2.99E+03 | 5.24E+02 | NA | NA |
| AB-007185 | 7.49E−02 | 5.77E−01 | 5.70E+00 | 1.56E+00 | 5.01E+03 | NA |
| AB-007186 | 2.87E−02 | 3.90E−02 | 1.49E+00 | 8.15E−01 | 1.06E+02 | NA |
| AB-007140 | 1.80E+00 | 1.80E+00 | 7.94E+02 | NA | NA | NA |
| AB-007141 | 7.62E−01 | 1.42E+00 | 6.29E+01 | 1.33E+04 | 1.60E+04 | 2.14E+04 |
| AB-007143 | 2.36E−02 | 5.31E−02 | 4.70E+00 | 3.60E+02 | 1.29E+03 | 2.09E+04 |
| AB-007144 | 5.77E−02 | 1.02E+00 | 4.09E+01 | NA | NA | NA |
| AB-007145 | 3.74E−02 | 9.73E−01 | 1.28E+01 | 4.86E+02 | 2.15E+04 | NA |
| AB-007146 | 1.74E−01 | 6.93E−01 | 1.44E+01 | 2.29E+03 | 1.29E+04 | 3.45E+04 |
| AB-007147 | 4.74E−02 | 4.66E−01 | 4.09E+00 | 1.61E+03 | 5.50E+03 | 2.47E+04 |
| AB-007148 | 4.99E−02 | 2.47E+00 | 1.35E+02 | NA | NA | NA |
| AB-007149 | 4.39E−02 | 9.02E−02 | 2.96E+01 | 1.79E+04 | 1.84E+04 | NA |
| AB-007151 | 1.07E−01 | 4.73E−01 | 8.79E+00 | 7.73E+01 | 2.30E+03 | NA |
| AB-007152 | 4.03E−02 | 5.60E−02 | 1.12E+01 | 5.56E+02 | 1.55E+04 | NA |
| AB-007153 | 1.29E−01 | 4.06E−02 | 1.43E+03 | 6.93E+00 | 5.91E+02 | NA |
| AB-007155 | 6.32E−02 | 4.28E−02 | 1.35E+00 | 2.64E−02 | 2.96E−01 | 5.90E+00 |
| AB-007156 | 2.16E−01 | 1.24E+00 | 2.60E+01 | 7.14E−01 | 1.00E+01 | 2.45E+04 |
| AB-007157 | 2.24E−02 | 2.76E−02 | 2.18E+02 | 8.36E−01 | 1.32E+00 | NA |
| AB-000222 | 8.59E−02 | 3.08E−02 | 6.49E−01 | 1.20E−01 | 2.64E−01 | 3.88E+02 |
| AB-007162 | 1.93E−02 | 5.71E−02 | 1.35E+00 | 6.79E−02 | 5.27E−01 | 2.51E+03 |
| AB-007164 | 7.12E−02 | 4.16E−01 | 9.48E−01 | 4.95E−01 | 5.87E−01 | 7.71E+02 |
| AB-007166 | 2.88E−01 | 4.49E−02 | 9.59E−01 | 4.17E−02 | 7.96E−01 | 8.98E+02 |
| AB-007167 | 4.71E−02 | 4.08E−01 | 5.80E−01 | 2.78E−01 | 5.26E−01 | 4.58E+02 |
| AB-007171 | 2.01E−02 | 2.41E−02 | 9.79E−01 | 3.01E−01 | NA | 1.66E+04 |
| AB-007172 | 1.56E−01 | 5.28E−02 | 7.25E+00 | 2.64E+00 | 1.88E+03 | NA |
| AB-007174 | 1.84E−02 | 4.09E−02 | 3.35E−01 | 8.85E−01 | 1.22E+01 | NA |
| AB-000337 | 2.51E−01 | 1.88E−01 | 1.37E+01 | 8.71E+00 | 8.89E+02 | 1.83E+04 |
| AB-007177 | 7.00E−02 | 6.57E−02 | 2.59E+01 | 4.14E+00 | 5.94E+01 | 6.44E+03 |
| AB-007179 | 3.73E−01 | 3.84E−02 | 6.66E+02 | 1.63E+00 | 2.13E+02 | NA |
| AB-007180 | 2.32E−01 | 2.77E−01 | 3.57E+02 | 7.26E+00 | 6.66E+03 | NA |
| AB-007181 | 1.73E−01 | 9.33E−01 | 9.88E+02 | 1.51E+01 | 1.15E+04 | NA |
| AB-007183 | 2.72E−01 | 1.64E−01 | 2.00E+03 | 3.94E+01 | NA | NA |

TABLE 8
Table 8 | Binding and in vivo pharmacology of lead mAbs

| | Liver burden inhibition | | | Liver burden mAb sera concentration | | |
|---|---|---|---|---|---|---|
| | Inhibition | AB-000317 | Comparison to AB- | [mAb]ₛₑᵣₐ/ | [mAb]ₛₑᵣₐ, avg. | [mAb]ₛₑᵣₐ, |
| mAb ID | (%) | normalized (%)^{a} | 000317^{b} | KD_{CSP-SPR} | (μg/ml) | avg. (M) |
| AB- | 88.9 | 95.5 | Comparable, p > 0.05 | 4,818 | 41.5 | 2.77E−07 |
| 007104 | | | | | | |
| AB- | 83.5 | 94.1 | Comparable, p > 0.05 | 1,129 | 46.1 | 3.07E−07 |
| 007088 | | | | | | |
| AB- | 82.5 | 88.6 | Worse, p < 0.05 | 18,857 | 59.3 | 3.95E−07 |
| 007103 | | | | | | |
| AB- | 97.3 | 101.9 | Better, p < 0.05 | 9,939 | 54.6 | 3.64E−07 |
| 000224 | | | | | | |
| AB- | 95.7 | 100.2 | Comparable, p > 0.05 | 865 | 42.1 | 2.81E−07 |
| 000399 | | | | | | |
| AB- | 97.5 | 102.1 | Comparable, p > 0.05 | 3,386 | 44.0 | 2.94E−07 |
| 007114 | | | | | | |
| AB- | 69.8 | 73.0 | Worse, p < 0.05 | 1,264 | 53.4 | 3.56E−07 |
| 001529 | | | | | | |
| AB- | 95.8 | 100.3 | Comparable, p > 0.05 | 10,271 | 48.0 | 3.20E−07 |
| 000364 | | | | | | |
| AB- | 56.2 | 59.5 | Worse, p < 0.05 | 739 | 57.4 | 3.83E−07 |
| 001480 | | | | | | |
| AB- | 94.3 | 101.3 | Comparable, p > 0.05 | 7,708 | 57.1 | 3.81E−07 |
| 007176 | | | | | | |
| AB- | 92.7 | NA | NA | 8,743 | 71.3 | 4.75E−07 |
| 000317 | | | | | | |
| AB- | 91.8 | 98.5 | Comparable, p > 0.05 | 6,937 | 55.7 | 3.71E−07 |
| 007131 | | | | | | |
| AB- | 88.3 | 94.8 | Comparable, p > 0.05 | 154 | 57.1 | 3.81E−07 |
| 007101 | | | | | | |
| AB- | 85.1 | 91.4 | Comparable, p > 0.05 | 322 | 57.4 | 3.83E−07 |
| 007161 | | | | | | |
| AB- | 80.3 | 86.3 | Worse, p < 0.05 | 12,303 | 47.9 | 3.19E−07 |
| 007121 | | | | | | |
| AB- | 44.1 | 47.4 | Worse, p < 0.05 | 6,695 | 53.3 | 3.55E−07 |
| 007173 | | | | | | |
| AB- | 90.0 | 95.3 | Comparable, p > 0.05 | 14,212 | 58.2 | 3.88E−07 |
| 007154 | | | | | | |
| AB- | 88.5 | 93.8 | Comparable, p > 0.05 | 2,291 | 48.3 | 3.22E−07 |
| 007083 | | | | | | |
| AB- | 86.1 | 90.9 | Worse, p < 0.05 | 371 | 58.3 | 3.89E−07 |
| 001558 | | | | | | |
| AB- | 48.2 | 53.3 | Worse, p < 0.05 | 1,184 | 66.4 | 4.42E−07 |
| 001387 | | | | | | |
| AB- | 88.7 | 98.7 | Comparable, p > 0.05 | 38,759 | 62.2 | 4.15E−07 |
| 007163 | | | | | | |
| AB- | 85.6 | 90.7 | Worse, p < 0.05 | 2,825 | 27.7 | 1.85E−07 |
| 007087 | | | | | | |
| AB- | 70.9 | 76.3 | Worse, p < 0.05 | ND | ND | ND |
| 000397 | | | | | | |
| AB- | 62.0 | 68.9 | Worse, p < 0.05 | 305 | 26.9 | 1.79E−07 |
| 001428 | | | | | | |
| AB- | 83.8 | 88.8 | Worse, p < 0.05 | 2,967 | 53.3 | 3.55E−07 |
| 007178 | | | | | | |
| AB- | 75.9 | 81.7 | Worse, p < 0.05 | ND | ND | ND |
| 000391 | | | | | | |
| AB- | 57.6 | 62.1 | Worse, p < 0.05 | ND | ND | ND |
| 007120 | | | | | | |
| AB- | 94.2 | 102.0 | Comparable, p > 0.05 | 7,917 | 49.8 | 3.32E−07 |
| 007110 | | | | | | |
| AB- | 91.9 | 96.2 | Comparable, p > 0.05 | 604 | 52.2 | 3.48E−07 |
| 000334 | | | | | | |
| AB- | 84.2 | 89.1 | Worse, p < 0.05 | 1,918 | 58.8 | 3.92E−07 |
| 007096 | | | | | | |
| AB- | 81.1 | 87.8 | Worse, p < 0.05 | 1,850 | 60.3 | 4.02E−07 |
| 007123 | | | | | | |
| AB- | 79.0 | 83.3 | Worse, p < 0.05 | 103 | 59.0 | 3.94E−07 |
| 001472 | | | | | | |
| AB- | 56.4 | 62.7 | Worse, p < 0.05 | 2,353 | 55.8 | 3.72E−07 |
| 007182 | | | | | | |
| AB- | 51.1 | 56.8 | Worse, p < 0.05 | 199 | 15.4 | 1.02E−07 |
| 001554 | | | | | | |
| AB- | 92.2 | 97.7 | Comparable, p > 0.05 | 1,859 | 56.0 | 3.73E−07 |
| 007130 | | | | | | |
| AB- | 86.8 | 96.5 | Comparable, p > 0.05 | 9,023 | 53.8 | 3.59E−07 |
| 007168 | | | | | | |
| AB- | 86.5 | 91.6 | Worse, p < 0.05 | 1,656 | 60.6 | 4.04E−07 |
| 000356 | | | | | | |
| AB- | 84.4 | 95.2 | Comparable, p > 0.05 | 19,835 | 55.9 | 3.73E−07 |
| 007150 | | | | | | |
| AB- | 82.1 | 88.3 | Worse, p < 0.05 | ND | ND | ND |
| 001455 | | | | | | |
| AB- | 78.0 | 86.7 | Worse, p < 0.05 | 10,936 | 53.8 | 3.59E−07 |
| 007175 | | | | | | |
| AB- | 75.5 | 83.5 | Worse, p < 0.05 | 2,191 | 56.1 | 3.74E−07 |
| 000373 | | | | | | |
| AB- | 72.9 | 78.5 | Worse, p < 0.05 | ND | ND | ND |
| 001533 | | | | | | |
| AB- | 50.1 | 55.4 | Worse, p < 0.05 | 1,867 | 70.3 | 4.68E−07 |
| 001458 | | | | | | |
| AB- | 93.4 | 103.8 | Comparable, p > 0.05 | 6,195 | 20.9 | 1.39E−07 |
| 000231 | | | | | | |
| AB- | 91.7 | 96.8 | Comparable, p > 0.05 | 1,666 | 58.4 | 3.89E−07 |
| 001396 | | | | | | |
| AB- | 90.7 | 96.1 | Comparable, p > 0.05 | 3,206 | 40.2 | 2.68E−07 |
| 007109 | | | | | | |
| AB- | 90.3 | 95.6 | Comparable, p > 0.05 | 2,857 | 66.9 | 4.46E−07 |
| 007090 | | | | | | |
| AB- | 85.9 | 90.9 | Comparable, p > 0.05 | 1,722 | 58.7 | 3.91E−07 |
| 007080 | | | | | | |
| AB- | 74.5 | 80.2 | Worse, p < 0.05 | ND | ND | ND |
| 007102 | | | | | | |
| AB- | 56.1 | 62.0 | Worse, p < 0.05 | 1,102 | 55.2 | 3.68E−07 |
| 000376 | | | | | | |
| AB- | 90.6 | 100.2 | Comparable, p > 0.05 | 3,148 | 58.6 | 3.91E−07 |
| 007076 | | | | | | |
| AB- | 83.7 | 88.3 | Worse, p < 0.05 | 263 | 55.1 | 3.67E−07 |
| 007106 | | | | | | |
| AB- | 49.4 | 52.1 | Worse, p < 0.05 | 338 | 58.9 | 3.93E−07 |
| 007107 | | | | | | |
| AB- | 93.6 | 101.3 | Comparable, p > 0.05 | 10,340 | 51.1 | 3.40E−07 |
| 007111 | | | | | | |
| AB- | 89.0 | 96.3 | Comparable, p > 0.05 | 2,271 | 30.2 | 2.01E−07 |
| 007159 | | | | | | |
| AB- | 88.1 | 97.5 | Comparable, p > 0.05 | 17,892 | 64.7 | 4.31E−07 |
| 007170 | | | | | | |
| AB- | 86.5 | 95.6 | Comparable, p > 0.05 | 5,638 | 67.7 | 4.51E−07 |
| 007165 | | | | | | |
| AB- | 79.8 | 86.4 | Worse, p < 0.05 | 1,257 | 50.7 | 3.38E−07 |
| 007115 | | | | | | |
| AB- | 72.3 | 81.5 | Worse, p < 0.05 | 6,074 | 60.8 | 4.05E−07 |
| 007138 | | | | | | |
| AB- | 66.3 | 73.7 | Worse, p < 0.05 | 5,646 | 25.0 | 1.66E−07 |
| 000327 | | | | | | |
| AB- | 94.9 | 99.4 | Comparable, p > 0.05 | 726 | 41.1 | 2.74E−07 |
| 007160 | | | | | | |
| AB- | 92.3 | 100.0 | Comparable, p > 0.05 | 3,992 | 53.0 | 3.53E−07 |
| 007112 | | | | | | |
| AB- | 85.7 | 92.2 | Worse, p < 0.05 | ND | ND | ND |
| 007142 | | | | | | |
| AB- | 71.1 | 76.6 | Worse, p < 0.05 | ND | ND | ND |
| 007126 | | | | | | |
| AB- | 94.4 | 100.0 | Comparable, p > 0.05 | 8,996 | 58.9 | 3.93E−07 |
| 007113 | | | | | | |
| AB- | 92.5 | 98.0 | Comparable, p > 0.05 | 3,618 | 55.1 | 3.68E−07 |
| 007108 | | | | | | |
| AB- | 91.6 | 99.2 | Comparable, p > 0.05 | 14,118 | 63.1 | 4.21E−07 |
| 007118 | | | | | | |
| AB- | 85.1 | 92.2 | Worse, p < 0.05 | 643 | 31.5 | 2.10E−07 |
| 007158 | | | | | | |
| AB- | 93.3 | 103.1 | Comparable, p > 0.05 | 25,685 | 67.3 | 4.49E−07 |
| 007169 | | | | | | |
| AB- | 73.1 | 82.4 | Worse, p < 0.05 | 12,667 | 56.3 | 3.76E−07 |
| 007139 | | | | | | |

TABLE 9

| SEQ | | | | Heavy chain | | | | Light chain | | | | | |
|---|---|---|---|---|---|---|---|---|---|---|---|---|---|
| ID | | | | SHM (nucleotide) | | SHM (amino acid) | | SHM (nucleotide) | | SHM (amino acid) | | LB Inhibition ^{c} | |
| NO | Binding peptide in SPR | Binding^{a} | Test^{b} | p | r | p | r | p | r | p | r | p | r |
| | CSP (3D7) | K_{off} | S | >0.8 | −0.02 | >0.7 | 0.03 | >0.08 | −0.17 | >0.07 | −0.17 | >0.2 | −0.18 |
| | CSP (3D7) | K_{off} | P | >0.3 | −0.09 | >0.7 | −0.03 | >0.1 | −0.14 | >0.1 | −0.14 | <0.01 | −0.37 |
| | CSP (3D7) | K_{off} | LR | >0.3 | −0.09 | nd | nd | nd | nd | nd | nd | <0.03 | −0.32 |
| 957 | NPNA3 | K_{off} | S | <0.0001 | −0.48 | <0.0001 | −0.52 | <0.0001 | −0.34 | <0.0002 | −0.32 | <0.0001 | −0.48 |
| 957 | NPNA3 | K_{off} | P | <0.0001 | −0.34 | <0.0004 | −0.30 | <0.03 | −0.19 | <0.05 | −0.17 | <0.0001 | −0.53 |
| 957 | NPNA3 | K_{off} | LR | <0.0001 | −0.48 | nd | nd | nd | nd | nd | nd | <0.0001 | −0.60 |
| | Junction | K_{off} | S | <0.03 | −0.26 | <0.04 | −0.25 | <0.003 | −0.36 | <0.006 | −0.33 | <0.05 | −0.31 |
| | Junction | K_{off} | P | <0.03 | −0.27 | <0.04 | −0.25 | <0.004 | −0.35 | <0.004 | −0.35 | <0.005 | −0.43 |
| | Junction | K_{off} | LR | <0.05 | −0.24 | nd | nd | nd | nd | nd | nd | <0.03 | −0.35 |
| 1183 | NPDPNANP2NVDP | K_{off} | S | <0.002 | −0.30 | <0.002 | −0.30 | >0.06 | −0.18 | >0.09 | −0.16 | >0.07 | −0.24 |
| 1183 | NPDPNANP2NVDP | K_{off} | P | <0.04 | −0.20 | >0.07 | −0.17 | >0.1 | −0.16 | >0.2 | −0.12 | <0.003 | −0.38 |
| 1183 | NPDPNANP2NVDP | K_{off} | LR | <0.002 | −0.30 | nd | nd | nd | nd | nd | nd | <0.005 | −0.37 |
| 1200 | NVDP3NANP2 | K_{off} | S | <0.02 | −0.24 | <0.05 | −0.18 | <0.04 | −0.20 | <0.05 | −0.18 | <0.002 | −0.40 |
| 1200 | NVDP3NANP2 | K_{off} | P | >0.4 | 0.06 | >0.2 | 0.10 | >0.6 | −0.04 | >0.9 | −0.01 | >0.3 | −0.13 |
| 1200 | NVDP3NANP2 | K_{off} | LR | >0.05 | −0.18 | nd | nd | nd | nd | nd | nd | <0.0007 | −0.42 |
| 1198 | NANP6 | K_{off} | S | >0.3 | −0.11 | >0.6 | −0.05 | >0.7 | 0.04 | >0.2 | 0.14 | >0.3 | 0.15 |
| 1198 | NANP6 | K_{off} | P | >0.06 | −0.20 | >0.1 | −0.15 | >0.1 | −0.16 | >0.1 | −0.15 | >0.6 | 0.09 |
| 1198 | NANP6 | K_{off} | LR | >0.2 | −0.12 | nd | nd | nd | nd | nd | nd | >0.7 | 0.05 |
| | CSP (3D7) | Kₒₙ | S | <0.008 | 0.22 | <0.0008 | 0.28 | <0.02 | 0.20 | <0.008 | 0.22 | >0.4 | 0.09 |
| | CSP (3D7) | Kₒₙ | P | <0.004 | 0.25 | <0.0007 | 0.28 | <0.02 | 0.21 | <0.007 | 0.23 | >0.4 | 0.09 |
| | CSP (3D7) | Kₒₙ | LR | <0.0005 | 0.42 | nd | nd | nd | nd | nd | nd | >0.5 | 0.07 |
| 957 | NPNA3 | Kₒₙ | S | <0.0001 | 0.47 | <0.0001 | 0.53 | <0.007 | 0.23 | <0.02 | 0.20 | <0.04 | 0.25 |
| 957 | NPNA3 | Kₒₙ | P | <0.0001 | 0.40 | <0.0001 | 0.47 | <0.05 | 0.17 | >0.07 | 0.15 | <0.02 | 0.31 |
| 957 | NPNA3 | Kₒₙ | LR | <0.0001 | 0.59 | nd | nd | nd | nd | nd | nd | <0.04 | 0.26 |
| | Junction | Kₒₙ | S | <0.004 | 0.35 | <0.0008 | 0.40 | <0.008 | 0.32 | <0.005 | 0.34 | >0.05 | 0.30 |
| | Junction | Kₒₙ | P | <0.03 | 0.27 | <0.008 | 0.32 | <0.03 | 0.28 | <0.004 | 0.35 | >0.09 | 0.26 |
| | Junction | Kₒₙ | LR | <0.003 | 0.45 | nd | nd | nd | nd | nd | nd | <0.04 | 0.32 |
| 1183 | NPDPNANP2NVDP | Kₒₙ | S | <0.0001 | 0.40 | <0.0001 | 0.44 | >0.1 | 0.13 | >0.06 | 0.17 | >0.1 | 0.20 |
| 1183 | NPDPNANP2NVDP | Kₒₙ | P | <0.001 | 0.31 | <0.0002 | 0.36 | >0.3 | 0.10 | >0.1 | 0.13 | <0.05 | 0.27 |
| 1183 | NPDPNANP2NVDP | Kₒₙ | LR | <0.0009 | 0.43 | nd | nd | nd | nd | nd | nd | >0.06 | 0.24 |
| 1200 | NVDP3NANP2 | Kₒₙ | S | <0.003 | 0.27 | <0.0002 | 0.33 | <0.02 | 0.22 | <0.02 | 0.22 | >0.4 | 0.09 |
| 1200 | NVDP3NANP2 | Kₒₙ | P | <0.004 | 0.26 | <0.0004 | 0.31 | >0.1 | 0.13 | >0.1 | 0.14 | >0.8 | 0.03 |
| 1200 | NVDP3NANP2 | Kₒₙ | LR | <0.002 | 0.38 | nd | nd | nd | nd | nd | nd | >0.5 | 0.08 |
| 1198 | NANP6 | Kₒₙ | S | <0.0009 | 0.28 | <0.0001 | 0.34 | <0.04 | 0.18 | >0.05 | 0.16 | >0.8 | −0.02 |
| | NANP6 | Kₒₙ | P | <0.009 | 0.22 | <0.0004 | 0.29 | <0.03 | 0.19 | <0.04 | 0.18 | >0.7 | 0.05 |
| 1198 | NANP6 | Kₒₙ | LR | <0.005 | 0.34 | nd | nd | nd | nd | nd | nd | >0.7 | −0.04 |
| 1198 | CSP (3D7) | K_{D} | S | <0.004 | −0.27 | <0.006 | −0.26 | <0.0003 | −0.34 | <0.0005 | −0.33 | >0.2 | −0.17 |
| | CSP (3D7) | K_{D} | P | >0.06 | −0.18 | >0.1 | −0.14 | <0.03 | −0.22 | <0.03 | −0.22 | >0.1 | −0.20 |
| | CSP (3D7) | K_{D} | LR | <0.002 | −0.46 | nd | nd | nd | nd | nd | nd | >0.1 | −0.22 |
| 957 | NPNA3 | K_{D} | S | <0.0001 | −0.53 | <0.0001 | −0.57 | <0.0001 | −0.33 | <0.0004 | −0.30 | <0.0003 | −0.43 |
| 957 | NPNA3 | K_{D} | P | <0.002 | −0.27 | <0.004 | −0.25 | <0.03 | −0.20 | <0.03 | −0.20 | <0.003 | −0.36 |
| 957 | NPNA3 | K_{D} | LR | <0.0001 | −0.70 | nd | nd | nd | nd | nd | nd | <0.0001 | −0.54 |
| | Junction | K_{D} | S | <0.008 | −0.32 | <0.005 | −0.34 | <0.005 | −0.34 | <0.005 | −0.34 | >0.06 | −0.29 |
| | Junction | K_{D} | P | <0.004 | −0.35 | <0.002 | −0.39 | <0.005 | −0.34 | <0.005 | −0.34 | <0.05 | −0.31 |
| | Junction | K_{D} | LR | <0.009 | −0.40 | nd | nd | nd | nd | nd | nd | <0.02 | −0.38 |
| 1183 | NPDPNANP2NVDP | K_{D} | S | <0.0001 | −0.38 | <0.0001 | −0.40 | >0.06 | −0.18 | <0.05 | −0.19 | >0.08 | −0.22 |
| 1183 | NPDPNANP2NVDP | K_{D} | P | <0.02 | −0.23 | <0.02 | −0.23 | <0.05 | −0.19 | >0.1 | −0.16 | <0.02 | −0.33 |
| 1183 | NPDPNANP2NVDP | K_{D} | LR | <0.002 | −0.40 | nd | nd | nd | nd | nd | nd | <0.009 | −0.34 |
| 1200 | NVDP3NANP2 | K_{D} | S | <0.0002 | −0.34 | <0.0002 | −0.34 | <0.002 | −0.28 | <0.003 | −0.27 | <0.002 | −0.40 |
| 1200 | NVDP3NANP2 | K_{D} | P | >0.3 | 0.09 | >0.2 | 0.10 | >0.6 | −0.04 | >0.8 | −0.02 | >0.3 | −0.11 |
| 1200 | NVDP3NANP2 | K_{D} | LR | <0.0005 | −0.43 | nd | nd | nd | nd | nd | nd | <0.006 | −0.35 |
| 1198 | NANP6 | K_{D} | S | <0.0005 | −0.37 | <0.0009 | −0.35 | <0.04 | −0.22 | >0.4 | −0.09 | >0.2 | 0.18 |
| 1198 | NANP6 | K_{D} | P | <0.002 | −0.33 | <0.003 | −0.32 | <0.02 | −0.27 | <0.008 | −0.28 | >0.4 | 0.13 |
| 1198 | NANP6 | K_{D} | LR | >0.1 | −0.27 | nd | nd | nd | nd | nd | nd | >0.5 | 0.10 |

| Functional activity in vivo vs. SHM | Test^{b} | p | r | p | r | p | r | p | r | | |
|---|---|---|---|---|---|---|---|---|---|---|---|
| Liver burden inhibition ^{c} | S | <0.02 | 0.30 | <0.0006 | 0.41 | <0.0002 | 0.44 | <0.0001 | 0.50 | NA | NA |
| Liver burden inhibition ^{c} | P | <0.0005 | 0.41 | <0.0001 | 0.48 | <0.0004 | 0.42 | <0.0001 | 0.47 | NA | NA |
| Liver burden inhibition ^{c} | LR | <0.0006 | 0.41 | <0.0001 | 0.48 | <0.0004 | 0.42 | <0.0001 | 0.47 | NA | NA |

nd , not done; NA, not applicable; LB, liver burden mouse model; NPNA3, NPNANPNANPNA; Junction, KQPADGNPDPNANPN; NPDPNANP2NVDP, NPDPNANPNVDPNANP; NVDP3NANP2, NVDPNANPNVDPNANPNVDP; NANP6, NANPNANPNANPNANPNANPNANP
^{a}SPR determined kₒₙ (kₐ), K_{off} (k_{d}), and K_{D}, see methods
^{b}S, Spearman correlation; P, Pearson correlation; LR, simple linear regression using log transformed K_{off}, Kₒₙ, or KD data
^{c} liver burden inhibition normalized to percent inhibition of AB-000317 tested in parallel
SEQ ID NO: 957, 1199, 1183, 1200, 1198)

Antibodies were also evaluated for binding to short (12-15 residues) and long (20-24 residues) peptides derived from the varied tetrapeptide-based homologous (NPNA3 (SEQ ID NO: 957) and NANP6 (SEQ ID NO: 1198) peptides) and heterologous epitopes (NPDPNANPNVDPNANP (SEQ ID NO: 1183), NVDP3NANP2 (SEQ ID NO: 1200) and junctional [KQPADJNPDPNANPN] (SEQ ID NO: 1201) peptides) of CSP (FIG. 1B). Among the strongest correlations observed were inverse relationships between SHM and k_{off} to the short major repeat peptide, the short minor repeat peptide, and the JR peptide (FIGS. 1C-1E). K_{off} rates calculated against the long homologous and heterologous peptides and against CSP either show weaker, but still statistically significant, correlations with SHM levels or altogether insignificant correlations (FIGS. 1F-1G, Table 9). Indeed, the strongest correlation was observed between SHM and binding rates to the short, homologous peptide even though the long version of the homologous peptide contains more repeats of the same epitope (Table 9).

Furthermore, correlations between SHM levels and binding rates to the JR peptide, which is heterologous to epitopes in RTS,S, were stronger than for the long homologous peptide (Table 9). These data indicate that B cell receptor maturation of these highly functional antibodies may have been preferentially driven by interactions with short versus long NANP (SEQ ID NO: 924) epitopes that benefited maturation to heterologous peptide sequences. These observations are consistent with reports that protective antibodies from anti-CSP immune responses can display promiscuous binding across distinct CSP epitopes, though other reports indicate that such promiscuity is not necessarily required for protection.

Anti-Sporozoite Activity Correlates with CSP-Peptide Binding and SHM Levels

Seventy antibodies, representing 33 of the 35 protective lineages evaluated in binding studies, were directly compared in intravenous sporozoite-challenge mouse models to the highly efficacious antibody AB-000317. Antibodies inhibited 44.1%-97.5% of sporozoite liver burden (47.4%-103.8% of AB-000317 inhibition, Tables 7 and 8). Overall, about half of the antibodies demonstrated comparable inhibition to AB-000317 (n=32), while the other half demonstrated significantly weaker inhibition (n=36), and one, AB-000224, showed activity that was superior to AB-000317 (FIGS. 2A-2B, Tables 7 and 8). Serum concentrations for most antibodies were at least 1000-fold higher than the CSP K_{D} of the respective antibodies (FIG. 2C, Tables 7 and 8), indicating that antibodies demonstrating weak inhibition were not likely due to low levels of circulating antibody. Lineages with at least one antibody that demonstrated activity consistent with AB-000317 were considered for further advancement.

To determine if RTS,S-driven affinity maturation contributed to antibody inhibition, we assessed whether percent inhibition compared to AB-000317 correlated with peptide binding kinetics or SHM levels. Relative activity was associated with slower k_{off} from CSP (FIG. 2D), with slower k_{off} from the short homologous peptide, NPNA3 (SEQ ID NO: 957) (FIG. 2E), and with slower k_{off} from the JR and the other short, heterologous peptide (FIG. 2F-2G, Table 9). Strikingly, no significant correlations were observed between inhibitory activity and binding kinetics with the long homologous peptide, NANP6 (SEQ ID NO: 1198) (P>0.3 [k_{off}]; P>0.7 [kₒₙ], Spearman and Pearson, Table 9), despite this peptide being the most representative of both RTS,S and CSP. Taken together, the data evaluating these inhibitory antibodies suggest that while binding to NPNA epitopes may be required, mutations which favor binding to heterologous peptides may be preferred over mutations that simply improve binding to the longer, homologous NPNA epitopes.

Affinity maturation via SHM likely underlies the correlations between in vivo function and binding kinetics, as inhibitory activity significantly correlates with heavy and light chain nucleotide and amino acid changes from germline (FIGS. 2H-2I, Table 9). Consistent with this observation, low SHM antibodies were more likely to demonstrate significantly weaker inhibition compared to AB-000317 than antibodies with higher mutational burden (86% [12/14], versus 44% [24/55], P=0.0069; Fisher's exact, two-sided). Taken together, these correlations between higher SHM levels and binding kinetics to homologous (FIGS. 1C and 1F) and heterologous epitopes (FIGS. 1D-1E, and 1G), and between higher SHM levels and inhibitory activity (FIG. 2H-2I), suggest that affinity maturation to epitopes of RTS,S includes bystander maturation to heterologous epitopes that may be functionally important.

Despite the correlations between SHM levels, inhibitory activity, and k_{off} from CSP and short peptides, some antibodies with high SHM levels are exceptions. In some cases, high SHM antibodies have relatively fast k_{off}, and slow kₒₙ, and are comparatively poor inhibitors like many of the low SHM antibodies (FIG. 2J). These antibodies may have resulted from inefficient affinity maturation, affinity maturation resulting in less inhibitory paratopes, and/or aberrant selection mechanisms limiting survival in and recall from memory (FIGS. 3E-3H). In other cases, some high SHM antibodies have relatively fast k_{off} and slow kₒₙ to short peptides but are still relatively good inhibitors despite their unfavorable binding kinetics (FIG. 2J). In these latter cases, affinity maturation toward antibody homotypic Fab-Fab interactions, not CSP epitopes, may contribute to the relatively strong activity. Inter-antibody binding events can contribute to anti-CSP-binding potency and increased functional activity, and have been reported for some antibodies described here. Such homotypic interactions may not be reflected in binding kinetics to short NPNA3 (SEQ ID NO: 957) peptides, which due to their short length, cannot sterically accommodate multiple simultaneous binding events. Indeed, four antibodies that have relatively fast k_{off} to short peptides, but are comparable to AB-000317 in activity, are from a lineage containing an antibody that binds via Fab-Fab homotypic interactions (AB-000399, FIG. 2J, red circles) Overall, the data are consistent with antibody affinity maturation via multiple, different modes of binding, and reveal several antibodies (>30) with activity comparable to that of AB-000317 and the potential to be developed into clinical leads.

Using the sporozoite liver burden data, the present example further down-selected 26 mAbs representing 15 lineages for evaluation in the parasitemia challenge model as an alternate endpoint for assessing in vivo function. This set included AB-000317, AB-000224, 23 other mAbs with liver burden inhibitory activity similar to AB-000317, and one mAb with weaker activity than AB-000317. All except two mAbs were significantly more likely to prevent parasitemia than the negative control. Seven mAbs, including AB-000224 and two other mAbs from the same lineage, displayed a trend towards superior protection versus AB-000317 (non-parametric log-rank hazard ratios <1 versus AB-000317, FIG. 2K; Tables 10-12). Serum concentrations for almost all mAbs (25/26) at the time of infection were at least 1000-fold higher than the respective mAb's KDCSP-SPR (Tables 7 and 8), indicating that mAbs more efficacious than AB-000317 were likely not missed due to low levels of circulating antibody.

TABLE 10

| | | Bite parasitemia vs. negative control | | |
|---|---|---|---|---|
| mAb ID | HR | 95% CI | HR, replicate | 95% CI, replicate |
| AB-007104 | 0.097 | 0.012, 0.81 | | |
| AB-007088 | 0.015 | 0.0017, 0.14 | 0.015 | 0.0017, 0.14 |
| AB-000224 | 0.015 | 0.0017, 0.14 | | |
| AB-000399 | 0.050 | 0.0062, 0.40 | | |
| AB-007114 | 0.072 | 0.0091, 0.58 | 0.029 | 0.0038, 0.22 |
| AB-000364 | 0.097 | 0.012, 0.81 | | |
| AB-000317 | 0.015 | 0.0017, 0.14 | 0.015 | 0.0017, 0.14 |
| AB-007154 | 0.050 | 0.0062, 0.40 | | |
| AB-007163 | 0.030 | 0.0036, 0.25 | | |
| AB-007110 | 0.050 | 0.0062, 0.40 | 0.030 | 0.0036, 0.25 |
| AB-000334 | 0.050 | 0.0062, 0.40 | | |
| AB-007130 | 0.015 | 0.0017, 0.14 | | |
| AB-007168 | 0.12 | 0.014, 1.1 | | |
| AB-007150 | 0.050 | 0.0062, 0.40 | | |
| AB-000231 | 0.12 | 0.014, 1.1 | 0.14 | 0.021, 0.89 |
| AB-001396 | 0.072 | 0.0091, 0.58 | 0.066 | 0.0097, 0.45 |
| AB-007090 | 0.050 | 0.0062, 0.40 | | |
| AB-007076 | 0.072 | 0.0091, 0.58 | | |
| AB-007111 | 0.030 | 0.0036, 0.25 | 0.010 | 0.0011, 0.094 |
| AB-007159 | 0.030 | 0.0036, 0.25 | | |
| AB-007170 | 0.050 | 0.0062, 0.40 | | |
| AB-007160 | 0.030 | 0.0036, 0.25 | | |
| AB-007112 | 0.030 | 0.0036, 0.25 | | |
| AB-007142 | 0.097 | 0.012, 0.81 | | |
| AB-007118 | 0.050 | 0.0062, 0.40 | | |
| AB-007169 | 0.050 | 0.0062, 0.40 | | |

TABLE 11

| | | Bite parasitemia vs. AB-000317 | | |
|---|---|---|---|---|
| mAb ID | HR | 95% CI | HR, replicate | 95% CI, replicate |
| AB-007104 | 3.5 | 0.98, 13 | | |
| AB-007088 | 0.64 | 0.10, 4.1 | 0.61 | 0.097, 3.8 |
| AB-000224 | 0.42 | 0.097, 1.8 | 0.74 | 0.15, 3.8 |
| AB-000399 | 4.3 | 1.1, 16 | | |
| AB-007114 | 1.3 | 0.37, 4.5 | 2.3 | 0.40, 13 |
| AB-000364 | 3.1 | 0.86, 11 | | |
| AB-000317 | | | | |
| AB-007154 | 3.2 | 0.71, 14 | | |
| AB-007163 | 2.0 | 0.50, 7.8 | | |
| AB-007110 | 0.37 | 0.050, 2.7 | 1.2 | 0.31, 4.3 |
| AB-000334 | 3.3 | 0.77, 14 | | |
| AB-007130 | 0.12 | 0.0070, 2.1 | | |
| AB-007168 | 5.1 | 1.3, 20 | | |
| AB-007150 | 0.97 | 0.27, 3.5 | | |
| AB-000231 | 2.9 | 0.81, 10 | 1.8 | 0.27, 12 |
| AB-001396 | 1.2 | 0.34, 4.4 | 3.5 | 0.64, 19 |
| AB-007090 | 1.7 | 0.36, 8.3 | | |
| AB-007076 | 3.6 | 0.78, 17 | | |
| AB-007111 | 0.12 | 0.0070, 2.1 | 1.8 | 0.43, 7.5 |
| AB-007159 | 2.1 | 0.51, 8.8 | | |
| AB-007170 | 0.72 | 0.12, 4.4 | | |
| AB-007160 | 1.6 | 0.35, 7.3 | | |
| AB-007112 | 1.7 | 0.42, 6.5 | | |
| AB-007142 | 3.5 | 0.98, 13 | | |
| AB-007118 | 1.7 | 0.35, 7.9 | | |
| AB-007169 | 1.6 | 0.24, 10 | | |

TABLE 12

| | Bite parasitemia mAb sera concentration | | |
|---|---|---|---|
| mAb ID | [mAb]ₛₑᵣₐ / KD_{CSP-SPR} | [mAb]ₛₑᵣₐ, avg. (μg/ml) | [mAb]ₛₑᵣₐ, avg. (M) |
| AB-007104 | 7,809 | 67.2 | 4.48E−07 |
| AB-007088 | 2,320 | 74.8 | 4.98E−07 |
| AB-000224 | 13,066 | 71.7 | 4.78E−07 |
| AB-000399 | 1,248 | 60.8 | 4.05E−07 |
| AB-007114 | 5,970 | 77.6 | 5.18E−07 |
| AB-000364 | 17,139 | 77.8 | 5.19E−07 |
| AB-000317 | 11,774 | 96.1 | 6.40E−07 |
| AB-007154 | 22,697 | 91.1 | 6.07E−07 |
| AB-007163 | 65,421 | 105.0 | 7.00E−07 |
| AB-007110 | 10,913 | 68.7 | 4.58E−07 |
| AB-000334 | 893 | 77.1 | 5.14E−07 |
| AB-007130 | 3,014 | 90.7 | 6.05E−07 |
| AB-007168 | 11,963 | 71.3 | 4.76E−07 |
| AB-007150 | 29,650 | 83.6 | 5.57E−07 |
| AB-000231 | 27,017 | 91.1 | 6.08E−07 |
| AB-001396 | 3,172 | 111.2 | 7.41E−07 |
| AB-007090 | 4,151 | 97.2 | 6.48E−07 |
| AB-007076 | 5,094 | 94.9 | 6.33E−07 |
| AB-007111 | 17,734 | 87.6 | 5.84E−07 |
| AB-007159 | 1,487 | 19.8 | 1.32E−07 |
| AB-007170 | 21,877 | 79.1 | 5.27E−07 |
| AB-007160 | 1,201 | 68.1 | 4.54E−07 |
| AB-007112 | 6,018 | 79.8 | 5.32E−07 |
| AB-007142 | 4,443 | 88.4 | 5.89E−07 |
| AB-007118 | 18,510 | 81.1 | 5.40E−07 |
| AB-007169 | 40,359 | 105.8 | 7.05E−07 |

### Example 2. Methods

The present example provides details on methods and experimental strategy adopted for the results illustrated in Example 1 above.

### Vaccinees, Plasmablast Isolation, IgG Sequencing

The collection of PBs was part of the phase 2a clinical trial of RTS,S/AS01 (Mosquirix™) vaccine with fractional third and fourth dose, of which the protocol was approved by the Walter Reed Army Institute of Research Institutional Review Board and the Western Institutional Review Board, and written informed consent was obtained from each subject before study procedures were initiated (ClinicalTrials.gov identifier: NCT01857869). Unique samples from trial participants obtained as PBMC for this study were used exhaustively and are not available.

Plasmablast isolation, cloning, and sequencing were performed using protocols publicly available as follows. PBMC were stained with the following mAbs: anti-CD3-FITC (BioLegend, cat #300406, clone UCHT1), anti-CD14-FITC (BioLegend, cat #325604, clone HCD14), anti-CD19-BV421 (BioLegend, cat #302234, clone HIB19), anti-CD20-PerCP/cy5.5 (BD, cat #340955, clone L27), anti-CD27-BV510 (BioLegend, cat #302836, clone O323), anti-CD38-PE/cy7 (BioLegend, cat #356607, clone HB-7), anti-IgA-FITC (Miltenyi, cat #130-113-175, clone IS11-8E10), anti-IgM-APC/cy7 (BioLegend, cat #314520, clone MHM-88). IgG+ PBs were single-cell sorted into 96-well PCR plates containing hypotonic buffer (330 nM dNTPs (NEB, cat #N0447L), 1 μg/ml BSA (NEB, cat #B9000S), 2 mM DTT (Sigma-Aldrich, cat #43816), 0.5% IGEPAL-430 (Sigma-Aldrich, cat #18896), and 200 unit/ml of Ribolock (Thermo Fisher Scientific, cat #EO0384)) based on gating for CD3-CD14-CD19+CD20-CD27+CD38++IgA-IgM-cells. Sequencing of IgG mRNA isolated from single-cell sorted PBs was performed with the following modifications: Desthiobiotinylated Oligo (dT) and Maxima H-Reverse Transcriptase (Thermo Fisher Scientific, cat #EP0753) were used for reverse transcription, cDNA was extracted using Dynabeads™ MyOne™ C1 Streptavidin beads (Thermo Fisher Scientific, cat #65001), concentrations of final NGS library preparations were determined using qPCR (KAPA SYBR® FAST qPCR Kit for Titanium, Kapa Biosystems), and natively paired IgG heavy and light chain amplicons were sequenced using Roche FLX+154 Titanium sequencing.

DNA barcode assignment and sequence assembly were performed as described: a minimum coverage of 10 reads was required for each heavy and light chain assembly to be acceptable. Both heavy and light chain reads were required to assemble unique contigs within a well. In cases where there was more than one contig, the well was rejected from consideration unless one of the contigs included at least 90% of the reads.

### Sequence, Lineage and Repertoire Feature Analyses

### Germline Assignments and Determination of SHM Levels

Variable (V), diversity (D) and joining (J) gene segment assignment and mutation identification were performed using an implementation of Somatic Diversification Analysis (SoDA) 65 and the IMGT human immunoglobulin germline database release, IMGT_20203166. SHM substitutions were counted for each antibody by aligning the heavy and light variable domains (start of framework 1 to end of framework 4) with a hidden Markov model that includes states for germline aligning regions (VDJ for heavy, VJ for light) and N nucleotide regions, and that counts the substitutions with respect to the germline sequence in just the aligned portion (not including the rare, observed indels). IgG Isotype (IgG1-4) assignment was performed by aligning the sequence 3′ of framework 4 to the IMGT human Ig constant region sequences from IMGT 20203166.

### CDR3 and Lineage Assignments

Complementarity determining region 3 (CDR3) sequences were defined by the Kabat annotation plus the first amino acid residue of framework 4, from which CDR3 lengths were calculated. Natively paired IgG sequence clones were assigned to the same lineage if they are derived from the same vaccinee, have the same IGHV and IGK/LV germline gene assignments, the same heavy chain CDR3 (H3) lengths, the same light chain CDR3 (L3) lengths, and at least 75% nucleic acid sequence identity across concatenated H3 and L3. In some cases, clones with IGHV3-33 and IGHV3-30 (germline genes that have high sequence identity) met all the criteria to be assigned to the same lineage, except for the difference in IGHV. In these cases, the clones were assigned to the same lineage. Lineages were assigned rank-size based on lineage frequency (number of PBs expressing clones in the lineage divided by the total number of PBs in the repertoire). In some cases, more than one lineage in a repertoire had the same rank-size, because the lineages had the same number of PBs.

### Convergence, Clonality and Recall

Two IgG clones were defined as convergent if they derived from different vaccinees, had the same IGHV and IGK/LV germline gene assignments, the same H3 lengths, the same light chain CDR3 (L3) lengths, and at least 85% BLOSUM62-weighted amino acid sequence identity between the concatenated H3 and L3. A lineage was defined as convergent with another lineage if they derived from different vaccinees and if there was at least one IgG clone in the first lineage that was convergent with at least one IgG clone in the second line-age. Clonality was summarized as the normalized entropy across all lineages in each P3D vaccinee repertoire. Specifically, the sum over i in 1 . . . . N of −(Ki/N*log(Ki/N)))/log(N), where N equals the number of lineages in the repertoire and Ki is the size of each lineage as the number of PB, with i being 1 to N. The normalized entropy takes values between 0 and 1 inclusive, where 0 implies a single lineage is totally dominant in abundance, and 1 implies that some sets of lineages N>1 are all equally abundant. Recalled lineages were defined as those lineages with at least one PB antibody clone observed in both P3D and P4D repertoires among vaccinees (n=17) from whom at least 100 PB were sequenced from P4D PBMC samples.

### Selection of Lineages and Clones for CSP ELISA Screening Library

PB lineages (n=369) for initial screening of CSP-reactivity were selected as described in the Example 2 and the specific clone from each selected lineage was chosen for recombinant expression and screening based on ≥1 of the following properties: i) the clone has the paired heavy and light chain amino acid sequence expressed by more PBs in the lineage than any other clone (“dominant clone”); and/or ii) the specific antibody sequence selected is convergent with at least one other specific antibody sequence in a lineage of another vaccinee with convergence defined by the method described above in, “Convergence, clonality and recall” (“convergent clone”); and/or iii) the clone is that identified by a “leafiest descent” of a phylogenetic tree of the lineage in which terminal clades of leaves are ranked according their number of leaves with the largest terminal clade defined as the leafiest (“leafiest decent clone”); and/or iv) the clone has the greatest number of nucleic acid mutations from germline among all clones in the lineage (“most mutated clone”). Three clones (0.8% of the screening library) did not meet any of these criteria due to errors that were not detected until after screening occurred. Proportions of antibodies that meet each criterion from protected and not protected vaccinees are not statistically different than the proportions seen for all antibodies in the screening library, Fisher's exact.

### Recombinant Antibody Production

Each antibody gene sequence was cloned into high expression mammalian vector. Briefly, variable regions sequences were synthesized and subcloned into expression vectors containing human heavy chain IgG1 and appropriate human kappa or lambda light chain constant region coding domain sequences. Each completed construct was sequence confirmed before proceeding to DNA plasmid production scale up. Suspension HEK293 cells were seeded in a shake flask and were expanded using serum-free chemically defined medium. On the day of transfection, the expanded cells were seeded into a new flask with fresh medium. Each DNA construct was transiently transfected into HEK293 cells using a cationic lipid transfection method. The cells were maintained as a batch-fed culture until the end of the production run. The conditioned media from the transient production run was harvested and clarified by centrifugation and filtration. The supernatant was loaded over a Protein A column pre-equilibrated with binding buffer. Washing buffer was passed through the column until the OD280 value (NanoDrop, Thermo Scientific) was measured to be zero. The target protein was eluted with a low pH buffer, fractions were collected, and the OD280 value of each fraction was recorded. Fractions containing the target protein were pooled and filtered through a 0.2 μm membrane filter. Purified antibodies were dialyzed against PBS and analyzed using LabChip GXII. Endotoxin measurements were performed using the chromogenic Limulus Amebocyte Lysate method with Pyrochrome (Associates of Cape Cod)

### CSP, NANP (SEQ ID NO: 924) Peptide and C-Terminal Peptide ELISA

Antibodies were mapped to CSP using a nearly full-length CSP, the (NANPx6) peptide and a CSP C-terminal peptide (Pf16). For the purpose of this study, antibodies were classified as ‘positive’, ‘negative’, or ‘indeterminant’. All antibodies were evaluated at either 0.15 or 0.04 μg/ml concentrations. The ELISA optical density (OD) was converted to a fold-induction over the average of four negative control antibodies run in each experiment. The range of OD responses observed in each experiment was then used to determine a borderline ‘indeterminant’ range for that experiment. Antibody ODs that failed to exceed average negative control antibody OD+3× standard deviations were classified as ‘negative’. Antibodies with ODs above average negative control antibody OD+3× standard deviations but not yet exceeding negative threshold+20% of OD range for the experiment were classified as ‘indeterminant’. Any antibody OD above the experimental ‘indeterminant’ threshold was classified as ‘positive.’

### HBsAg ELISA

The MONOLISA Anti-HBs EIA kit [Bio-Rad Cat. No. 25220] was used for the determination of HBsAg reactivity of antibodies. A four-point 1:3 dilution series was prepared for each tested article in duplicate. The maximal stock input was 10% of the purified total volume for each of the 139 tested antibodies. The starting concentration for each tested anti-body was individually adjusted to 300 nM if it required <10% of the purified total volume. Otherwise, the starting concentration was based on the protein amount included in 10% of the purified total volume. The Cutoff Calibrator from the kit was performed in quadruplicate, while both negative controls were each performed in duplicate. A four-point, 1:3 dilution dose-response curve of PC3, starting at 150 μM, was also performed in duplicate. Antibodies were considered HBsAg-positive if the signal met the Cutoff Calibrator Criteria for at least one concentration ≤30 nM. Antibodies were considered “borderline” HBsAg-reactive if the signal was negative at concentrations tested ≤30 nM but did meet the Cutoff Calibrator Criteria for any concentration >30 nM. Antibodies were considered negative if the signal did not meet the Cutoff Calibrator Criteria for any concentration tested.

Selection of mAbs for Initial Characterization in the Mouse Sporozoite Challenge Model

Among the 102 antibodies that were reactive in the NANP6 (SEQ ID NO: 1198) peptide ELISA (FIG. 1D), 69 were originally selected to be screened in vivo based on representation of IGHV, vaccine protection status, and levels of SHM. Specifically, the 102 antibodies were divided into 11 groups based on the 11 different IGHV expressed among them (IGHV1-2, 1-69, 1-8, 3-15, 3-23, 3-30, 3-33, 3-48, 3-49, 3-7, and 5-51), and at least half of the mAbs in each set were selected including mAbs from both protected (n=26) and unprotected (n=8) vaccines. Only antibodies with high levels of SHM (≥20 nucleotide mutations from germline per antibody) were selected, except for the sets that contained IGHV3-33 and 3-49 from which some antibodies with low levels of SHM (<20 nucleotide mutations from germline per antibody) were also included. Two antibodies from these selections did not express enough material to be tested in vivo (the only antibody that contained IGHV3-23 and one of the two antibodies that contained IGHV5-51).

Among the 20 antibodies that were reactive in the C-terminal (Pf16) peptide ELISA, 11 of the 12 that were from protected vaccines were originally selected to be screened in vivo. These included all of the IGHV germline genes observed among C-terminal (Pf16) binders from protected vaccinees (IGHV3-11, IGHV3-21, IGHV3-30, IGHV3-48, and IGHV4-59). One antibody from these selections did not express enough material to be tested in vivo (the only mAb that contained IGHV3-11).

### Selection of Antibodies for Library Used in Surface Plasmon Resonance (SPR) Binding Analyses

Among the 52 mAbs, representing 52 unique lineages, that demonstrated ≥90% inhibition in the initial sporozoite liver burden mouse model screen, all of those that originated from protected vaccinees (n=36) were selected for further binding analyses except for one mAb that was observed to be reactive in the HBsAg ELISA (AB-000239). For each of these 35 antibodies, representing 35 unique lineages, the original hit antibody was picked if it contained no high-risk liability (i.e. odd number of cysteines in CDRs, any canonical N-linked glycosylation sites in CDRs, Fv net charge (at pH 5.5) >9, or hydrophobicity index >6.5). In cases where the original hit had ≥1 liabilities, other clones were selected from the lineage from either P3D or P4D PB repertoires. In addition, more than one clone was selected from lineages with extensive inter-clonal sequence diversity. This was done using the following algorithm: i) query each clone of lineage in leafiest-descent order; ii) skip any clones with >0 high-risk liabilities; iii) skip any clones that are too close to any already-picked clones with the distance between clones determined as the fraction of CDR amino acids that are non-conserved between the clones using BLOSUM62 matrix (≤0); iv) adjust the distance that is acceptable between clones so that a total of 141 clones were ultimately selected from the 35 lineages.

### High Throughput SPR

The binding kinetics measurements of antibody interaction with CSP antigens were made using the Carterra LSA high throughput SPR platform and CMD200M sensor chips (Carterra) at 25° C. The antigen panel included a recombinant CSP and synthetic peptides NPNA3 (NPNANPNANPNA) (SEQ ID NO: 957), NANP6 (NANPNANPNANPNANPNANPNANP) (SEQ ID NO: 1198), junction peptide (KQPADGNPDPNANPN) (SEQ ID NO: 1199), NPDPNANP2NVDP (NPDPNANPNVDPNANP) (SEQ ID NO: 1183) and NVDP3NANP2 (NVDPNANPNVDPNANPNVDP) (SEQ ID NO: 1200) that were custom made by CPC scientific. Except for the NANP6 (SEQ ID NO: 1198), all other peptides were acetylated at N-termini and amidated at C-termini. NANP6 (SEQ ID NO: 1198) contained an N-terminal biotin-aminohexanoic acid tag and an unmodified C-terminus. Two microfluidic modules, a 96-channel print-head (96PH) and a single flow cell (SFC), were used to deliver liquids onto the sensor chip. A single analyte antigen was titrated in each assay against the immobilized antibodies.

The immobilization of antibodies onto the CMD200M chips depended on the type of analyte used during titration. In assays involving recombinant CSP used as an analyte, a goat anti-Human IgG Fc antibody (Millipore) was first immobilized onto the chip through amine-coupling. The chip was first activated by 100 mM N-Hydroxysuccinimide (NHS) and 100 mM 1 Ethyl-3-(3-dimethylaminopropyl) carbodiimide hydrochloride (EDC) (GE healthcare, mixed 1:1:1 with 0.1 M MES buffer at pH 5.5) for 400 seconds, followed by immobilization of anti-Human IgG Fc (in 10 mM Sodium Acetate at pH 4.5) at 50 μg/ml for 900 seconds. Unreactive esters were quenched with a 400-second injection of 1 M Ethanolamine-HCl at pH 8.5. The chip was then exposed to double pulses (30 seconds per pulse) of 10 mM Glycine at pH 2.0. The CSP-specific antibodies were then captured on anti-Hu IgG Fc surfaces by injection of antibodies at 10 μg/ml or 5 μg/ml concentration for 400 seconds using the 96PH, with 1×HBSTE buffer (10 mM HEPES pH 7.4, 150 mM NaCl, 3 mM EDTA and 0.01% Tween-20) as running buffer and antibody diluent. If CSP-peptide antigens were used as analytes, the chip was activated by NHS/EDC for 400 seconds, followed by direct immobilization of CSP-specific antibodies (in 10 mM Sodium Acetate at pH 4.5) injected at 10 μg/ml or 5 μg/ml concentrations for 400 seconds using the 96PH. Unreactive esters were then quenched with a 400-second injection of 1 M ethanolamine-HCl at pH 8.5. Then 45 cycles of 1×HBSTE buffer injections with 1×HBSTE also as running buffer were used to wash off non-specifically bound IgG overnight from the sensor chip surface without using regeneration buffer. Except for the capture of antibodies by anti-Human IgG Fc and washing of non-specifically bound IgG, the running buffer was 10 mM MES buffer at pH 5.5 with 0.01% Tween-20. Unless specified above, the steps were done using the SFC.

During the initial screening, each antibody at a given diluted concentration was immobilized onto two separate spots of the same chip, enabling duplicate measurements of binding kinetics. For binding measurements of engineered variants, each antibody was immobilized onto three different spots enabling triplicate measurements.

A two-fold dilution series of the antigen was prepared in 1×HBSTE buffer. The top concentration for full-length CSP and all CSP-peptide antigens was 8 μg/ml (0.25 μM for CSP, 2.92 μM for NANP6 (SEQ ID NO: 1198), 6.41 μM for NPNA3 (SEQ ID NO: 957), 3.76 μM for NVDP3NANP2 (SEQ ID NO: 1200), 4.70 μM for NPDPNANPNVDPNANP (SEQ ID NO: 1183), 5.03 μM for N-Interface). The antigen at different concentrations was then injected using SFC onto the chip surface from the lowest to the highest concentration without regeneration, including eight injections of buffer before the lowest non-zero concentration for signal stabilization. For each concentration, the data collection involved 120 seconds of baseline step and 900 seconds of dissociation steps. The duration of the association step was 240 seconds for full-length CSP and NANP6 (SEQ ID NO: 1198) antigens and 300 seconds for all other CSP-peptide antigens. For all assays, the running buffer for titration was 1×HBSTE.

The kinetics titration data collected were first pre-processed in the NextGenKIT (Carterra) software, including reference subtraction, buffer subtraction and data smoothing. The data were then exported and analyzed using the TitrationAnalysis tool developed in-house68. The specific binding time courses for each antibody construct immobilized on different spots were fitted to a 1:1 Langmuir model to derive ka (“kon”), kd (“koff”) and KD values. The KD values determined for antigens with multiple repeats of epitopes include the avidity effect. The average values of duplicate measurements were reported for each antibody-antigen pair from the initial screening panel. For the engineered variants, average values of triplicate measurements were reported with the following data acceptance criteria: i) standard error of the estimated kon, koff and KD in each replicate ≤20% and ii) fold-change for all three parameters within the triplicate ≤3.

### In Vivo Functional Assessments

The assays using mice were performed in strict accordance with the recommendations in the Guide for the Care and Use of Laboratory Animals of the National Institutes of Health. The protocol was approved by the Animal Care and Use Committee of the Johns Hopkins University, protocols number MO18H419 and MO21H417

### Sporozoite-Challenge Liver Burden Mouse Models

Initial functional screening was performed in mice that were immunized with anti-CSP antibodies and challenged 16 h later intravenously with 2,000 P. berghei transgenic sporozoites expressing the full P. falciparum CSP. Forty-two hours later, mice were euthanized, and their livers excised to extract RNA, to perform RTqPCR to measure plasmodial 18s rRNA, using forward primer 5′-TGGGAGATTGGTTTTGACGTTTATGT-3′ (SEQ ID NO: 1196) and reverse primer 5′-AAGCATTAAATAAAGCGAATACATCCTTAC-3′ (SEQ ID NO: 1197). Parasite loads were expressed as P. berghei 18s rRNA copy number and percent inhibition of load was calculated compared to negative controls.

All other liver burden assays were performed as described by Flores-Garcia et al 20. Briefly, Anopheles stephensi mosquitoes infected with transgenic P. berghei sporozoites expressing the P. falciparum CSP and luciferase were maintained in an incubator at 19° C. Sporozoites from mosquitoes were harvested at days 20-23 post-infection in HBSS-FBS 2%. Mice were administered (passively immunized) with 100 μg of antibody per mouse and challenged 16 hours later with 2000 sporozoites injected intravenously. Control mice received either irrelevant or no antibodies. Forty-two hours after the challenge, mice were injected with 100 μl of D-luciferin (30 mg/ml), anaesthetized with isoflurane and the bioluminescence ex-pressed by the parasites in the liver was measured using an IVIS Spectrum imager, Perkin Elmer. Results are expressed as photons/second.

### Mosquito-Bite Challenge Parasitemia Mouse Models

The mosquito-bite challenge to evaluate sterile protection was performed as described by Flores-Garcia, et al. Briefly, 7-8 weeks old mice were passively immunized with 150 μg/mouse of the indicated antibody, 16 hours later, mice were anesthetized and placed for 10 minutes on the top of cages containing five mosquitoes infected with P. berghei sporozoites expressing the P. falciparum CSP and luciferase. From days 4 to 10 after the challenge, blood smears stained with Giemsa were observed under a light microscope to deter-mine the appearance of parasitemia. Control mice receiving irrelevant or no antibodies were challenged similarly.

### Assessment of mAb Concentrations in Sera Samples

Capture antibody (AffiniPure mouse anti-human IgG Fc fragment specific, Jackson ImmunoResearch #209-005-098) was adsorbed overnight at 21° C. onto 96-well polystyrene microplates (Immuno Plate Maxisorp, ThermoFisher Scientific #439454) in PBS [Dulbecco's Phosphate Buffered Saline, without Calcium and Magnesium, sterile pH 7.4, Wisent #311-425-LL]) and then washed 3 times in wash buffer (0.05% TWEEN 20 [Sigma #P2287] in PBS). Microplates were blocked for 1 hour at 21° C. with assay buffer (1% bovine serum albumin [Blocker BSA, Thermofisher #37525] in wash buffer). After washing with wash buffer 3 times, serum samples from mice and control-standards were added in duplicate at serially dilutions in normal mouse serum that were then further diluted 100-fold in assay buffer prior to incubation for 1 hour at 21° C. Control-standards consisted of AB-000317 serially diluted at 1.6-fold increments from 0.146-25.6 μg/ml. Microplates were then washed 3 times with wash buffer and incubated with mouse, monoclonal anti-human IgG antibody conjugated to horse radish peroxidase (HRP-conjugated clone JDC-101, Southern Biotech #9040-05) in assay buffer for 1 hour at 21° C. Following 3 washes with wash buffer, peroxidase substrate, TMB (Bio-Rad #1721068), was added, followed by a stop solution (TMB stop solution [650 nm], Southern Biotech #0413-01L). Absorbance was measured at 650 nm (Molecular Devices microplate reader using SoftMaxPro GxP version 6.5.1) and concentrations of human IgG in test samples were calculated using the standard curve generated from the control antibody by interpolation of the OD values on the 5-parameter logistic standard curve (derived from the mean ODs of duplicate standard samples) and adjusted according to their corresponding dilution factor. Final sample concentrations were then determined by calculating the average of all concentrations, for a sample, obtained within the range of the standard curve.

### REFERENCES

World Health Organization. World malaria report 2022. https://apps.who.int/iris/handle/10665/365169 (2022).
Sinnis, P. & Fidock, D. A. The RTS,S vaccine—a chance to regain the upper hand against malaria? Cell 185, 750-754 (2022).
March, S. et al. A microscale human liver platform that supports the hepatic stages of Plasmodium falciparum and vivax. Cell Host Microbe 14, 104-115 (2013).
Campo, J. J. et al. RTS,S vaccination is associated with serologic evidence of decreased exposure to Plasmodium falciparum liver- and blood-stage parasites. Mol Cell Proteomics 14, 519-531 (2015).
RTS,S Clinical Trials Partnership. Efficacy and safety of RTS,S/AS01 malaria vaccine with or without a booster dose in infants and children in Africa: final results of a phase 3, individually randomised, controlled trial. Lancet 386, 31-45 (2015).
World Health Organization. Global technical strategy for malaria 2016-2030, 2021 update. (World Health Organization, 2021).
Daily, J. P. Monoclonal antibodies—A different approach to combat malaria. N Engl J Med 387, 460-461 (2022).
Gaudinski, M. R. et al. A monoclonal antibody for malaria prevention. N Engl J Med 385, 803-814 (2021).
Kayentao, K. et al. Safety and efficacy of a monoclonal antibody against malaria in Mali. N Engl J Med 387, 1833-1842 (2022).
Birkett, A., Miller, R. S. & Soisson, L. A. The Importance of Exercising Caution When Comparing Results from Malaria Vaccines Administered on the EPI Schedule and on a Seasonal Schedule. Am J Trop Med Hyg 107, 1356 (2022).
Wu, R. L. et al. Low-dose subcutaneous or intravenous monoclonal antibody to prevent malaria. N Engl J Med 387, 397-407 (2022).
Dame, J. B. et al. Structure of the gene encoding the immunodominant surface antigen on the sporozoite of the human malaria parasite Plasmodium falciparum. Science 225, 593-599 (1984).
Zavala, F., Cochrane, A. H., Nardin, E. H., Nussenzweig, R. S. & Nussenzweig, V. Circumsporozoite proteins of malaria parasites contain a single immunodominant region with two or more identical epitopes. Journal of Experimental Medicine 157, 1947-1957 (1983).
Wang, L. T. et al. A potent anti-malarial human monoclonal antibody targets circumsporozoite protein minor repeats and neutralizes sporozoites in the liver. Immunity 53, 733-744.e8 (2020).
Kisalu, N. K. et al. A human monoclonal antibody prevents malaria infection by targeting a new site of vulnerability on the parasite. Nat Med 24, 408-416 (2018).
Zeeshan, M. et al. Genetic variation in the Plasmodium falciparum circumsporozoite protein in India and its relevance to RTS,S malaria vaccine. PLoS One 7, e43430 (2012).
Weber, J. L. & Hockmeyer, W. T. Structure of the circumsporozoite protein gene in 18 strains of Plasmodium falciparum. Mol Biochem Parasitol 15, 305-316 (1985).
Oyen, D. et al. Structural basis for antibody recognition of the NANP (SEQ ID NO: 924) repeats in Plasmodium falciparum circumsporozoite protein. Proc. Natl. Acad. Sci. U.S.A. 114, E10438-E10445 (2017).
Flores-Garcia, Y. et al. Optimization of an in vivo model to study immunity to Plasmodium falciparum pre-erythrocytic stages. Malar J 18, 426 (2019).
Otsubo, R. & Yasui, T. Monoclonal antibody therapeutics for infectious diseases: Beyond normal human immunoglobulin. Pharmacology & Therapeutics 240, 108233 (2022).
Kelley, B., Renshaw, T. & Kamarck, M. Process and operations strategies to enable global access to antibody therapies. Biotechnol Progress 37, e3139 (2021).
World Health Organization. Monoclonal antibodies for malaria prevention: preferred product characteristics and clinical development considerations. https://www.who.int/publications/i/item/9789240070981.
Regules, J. A. et al. Fractional third and fourth dose of RTS,S/AS01 malaria candidate vaccine: A phase 2a controlled human malaria parasite infection and immunogenicity study. J Infect Dis. 214, 762-771 (2016).
Triller, G. et al. Natural parasite exposure induces protective human anti-malarial antibodies. Immunity 47, 1197-1209.e10 (2017).
Tan, J. et al. A public antibody lineage that potently inhibits malaria infection through dual binding to the circumsporozoite protein. Nat Med 24, 401-407 (2018).
Imkeller, K. et al. Antihomotypic affinity maturation improves human B cell responses against a repetitive epitope. Science 360, 1358-1362 (2018).
Murugan, R. et al. Evolution of protective human antibodies against Plasmodium falciparum circumsporozoite protein repeat motifs. Nat Med 26, 1135-1145 (2020).
Cox, R. J. et al. An early humoral immune response in peripheral blood following parenteral inactivated influenza vaccination. Vaccine 12, 993-999 (1994).
Radbruch, A. et al. Competence and competition: the challenge of becoming a long-lived plasma cell. Nat Rev Immunol 6, 741-750 (2006).
Nutt, S. L., Hodgkin, P. D., Tarlinton, D. M. & Corcoran, L. M. The generation of antibody-secreting plasma cells. Nat Rev Immunol 15, 160-171 (2015).
Murugan, R. et al. Clonal selection drives protective memory B cell responses in controlled human malaria infection. Sci. Immunol. 3, eaap8029 (2018).
Foquet, L. et al. Vaccine-induced monoclonal antibodies targeting circumsporozoite protein prevent Plasmodium falciparum infection. J. Clin. Invest. 124, 140-144 (2014).
Flores-Garcia, Y. et al. The P. falciparum CSP repeat region contains three distinct epitopes required for protection by antibodies in vivo. PLoS Pathog 17, e1010042 (2021).
Raghunandan, R. et al. Characterization of two in vivo challenge models to measure functional activity of monoclonal antibodies to Plasmodium falciparum circumsporozoite protein. Malar J 19, 113 (2020).
Suscovich, T. J. et al. Mapping functional humoral correlates of protection against malaria challenge following RTS,S/AS01 vaccination. Sci. Transl. Med. 12, eabb4757 (2020).
Chatterjee, D. et al. Avid binding by B cells to the Plasmodium circumsporozoite protein repeat suppresses responses to protective subdominant epitopes. Cell Reports 35, 108996 (2021).
Livingstone, M. C. et al. In vitro and in vivo inhibition of malaria parasite infection by monoclonal antibodies against Plasmodium falciparum circumsporozoite protein (CSP). Sci Rep 11, 5318 (2021).
Oyen, D. et al. Cryo-EM structure of P. falciparum circumsporozoite protein with a vaccine-elicited antibody is stabilized by somatically mutated inter-Fab contacts. Sci. Adv. 4, eaau8529 (2018).
Pholcharee, T. et al. Diverse antibody responses to conserved structural motifs in Plasmodium falciparum circumsporozoite protein. Journal of Molecular Biology 432, 1048-1063 (2020).
Pholcharee, T. et al. Structural and biophysical correlation of anti-NANP antibodies with in vivo protection against P. falciparum. Nat Commun 12, 1063 (2021).
Martin, G. M. et al. Affinity-matured homotypic interactions induce spectrum of PfCSP-antibody structures that influence protection from malaria infection. 2022.09.20.508747 Preprint at https://www.biorxiv.org/content/10.1101/2022.09.20.508747v1 (2022).
Dennison, S. M. et al. Breadth of human monoclonal antibodies isolated from RTS,S/AS01 vaccinees binding to Plasmodium falciparum circumsporozoite protein antigens. Biophysical Journal 116, 480a (2019).
Wang, L. T. et al. Protective effects of combining monoclonal antibodies and vaccines against the Plasmodium falciparum circumsporozoite protein. PLoS Pathog 17, e1010133 (2021).
Langowski, M. D. et al. Restricted valency (NPNA) n repeats and junctional epitope-based circumsporozoite protein vaccines against Plasmodium falciparum. NPJ Vaccines 7, 13 (2022).
McNamara, H. A. et al. Antibody feedback limits the expansion of B cell responses to malaria vaccination but drives diversification of the humoral response. Cell Host & Microbe 28, 572-585.e7 (2020).
Vijayan, K. et al. Antibody interference by a non-neutralizing antibody abrogates humoral protection against Plasmodium yoelii liver stage. Cell Reports 36, 109489 (2021).
Vijay, R. et al. Infection-induced plasmablasts are a nutrient sink that impairs humoral immunity to malaria. Nat Immunol 21, 790-801 (2020).
Kucharska, I. et al. High-density binding to Plasmodium falciparum circumsporozoite protein repeats by inhibitory antibody elicited in mouse with human immunoglobulin repertoire. PLoS Pathog 18, e1010999 (2022).
Xu, Y. et al. Structure, heterogeneity and developability assessment of therapeutic antibodies. mAbs 11, 239-264 (2019).
Fernández-Quintero, M. L. et al. Assessing developability early in the discovery process for novel biologics. MAbs 15, 2171248 (2023).
Gunasekaran, K., Hagler, A. T. & Gierasch, L. M. Sequence and structural analysis of cellular retinoic acid-binding proteins reveals a network of conserved hydrophobic interactions. Proteins: Structure, Function, and Bioinformatics 54, 179-194 (2004).
Kerwin, B. A. et al. Framework mutations of the 10-1074 bnAb increase conformational stability, manufacturability, and stability while preserving full neutralization activity. J Pharm Sci 109, 233-246 (2020).
Zalevsky, J. et al. Enhanced antibody half-life improves in vivo activity. Nat Biotechnol 28, 157-159 (2010).
Kurtovic, L. et al. Multifunctional antibodies are induced by the RTS,S malaria vaccine and associated with protection in a phase 1/2a trial. The Journal of Infectious Diseases 224, 1128-1138 (2021).
White, M. T. et al. The Relationship between RTS,S Vaccine-Induced Antibodies, CD4+ T Cell Responses and Protection against Plasmodium falciparum Infection. PLoS ONE 8, e61395 (2013).
Kester, K. E. et al. A phase I/IIa safety, immunogenicity, and efficacy bridging randomized study of a two-dose regimen of liquid and lyophilized formulations of the candidate malaria vaccine RTS,S/AS02A in malaria-naïve adults. Vaccine 25, 5359-5366 (2007).
Ockenhouse, C. F. et al. Ad35.CS.01-RTS,S/AS01 Heterologous Prime Boost Vaccine Efficacy against Sporozoite Challenge in Healthy Malaria-Naïve Adults. PLoS ONE 10, e0131571 (2015).
Chaudhury, S. et al. The biological function of antibodies induced by the RTS,S/AS01 malaria vaccine candidate is determined by their fine specificity. Malar J 15, 301 (2016).
Pallikkuth, S. et al. A delayed fractionated dose RTS,S AS01 vaccine regimen mediates protection via improved T follicular helper and B cell responses. eLife 9, e51889 (2020).
Dennison, S. M. et al. Magnitude, Specificity, and Avidity of Sporozoite-Specific Antibodies Associate With Protection Status and Distinguish Among RTS,S/AS01 Dose Regimens. Open Forum Infectious Diseases 8, ofaa644 (2021).
Langowski, M. D. et al. Optimization of a Plasmodium falciparum circumsporozoite protein repeat vaccine using the tobacco mosaic virus platform. Proc Natl Acad Sci USA 117, 3114-3122 (2020).
Chatterjee, D. & Cockburn, I. A. The challenges of a circumsporozoite protein-based malaria vaccine. Expert Review of Vaccines 20, 113-125 (2021).
Cockburn, I. A. & Seder, R. A. Malaria prevention: from immunological concepts to effective vaccines and protective antibodies. Nat Immunol 19, 1199-1211 (2018).
Wang, L. T. et al. The light chain of the L9 antibody is critical for binding circumsporozoite protein minor repeats and preventing malaria. Cell Reports 38, 110367 (2022).
Calvo-Calle, J. M., Mitchell, R., Altszuler, R., Othoro, C. & Nardin, E. Identification of a neutralizing epitope within minor repeat region of Plasmodium falciparum CS protein. npj Vaccines 6, 10 (2021).
Hou, N. et al. Low-Complexity Repetitive Epitopes of Plasmodium falciparum Are Decoys for Humoural Immune Responses. Front Immunol 11, 610 (2020).
Rénia, L. & Goh, Y. S. Malaria Parasites: The Great Escape. Front. Immunol. 7, (2016).
Schofield, L. The circumsporozoite protein of Plasmodium: a mechanism of immune evasion by the malaria parasite? Bull World Health Organ 68, 66-73 (1990).
Schofield, L. & Uadia, P. Lack of Ir gene control in the immune response to malaria. I. A thymus-independent antibody response to the repetitive surface protein of sporozoites. 9.
Raghavan, M. et al. Antibodies to repeat-containing antigens in Plasmodium falciparum are exposure-dependent and short-lived in children in natural malaria infections. Elife 12, e81401 (2023).
Friedman-Klabanoff, D. J. et al. Epitope-Specific Antibody Responses to a Plasmodium falciparum Subunit Vaccine Target in a Malaria-Endemic Population. The Journal of Infectious Diseases 223, 1943-1947 (2021).
Anders, R. F. et al. Antigenic Repeat Structures in Proteins of Plasmodium Falciparum. in Ciba Foundation Symposium 119-Synthetic Peptides as Antigens 164-183 (John Wiley & Sons, Ltd). doi: 10.1002/9780470513286.ch10.
Dennison, S. M. et al. Qualified Biolayer Interferometry Avidity Measurements Distinguish the Heterogeneity of Antibody Interactions with Plasmodium falciparum Circumsporozoite Protein Antigens. J. I. 201, 1315-1326 (2018).
Kucharska, I. et al. Structural ordering of the Plasmodium berghei circumsporozoite protein repeats by inhibitory antibody 3D11. eLife 9, e59018 (2020).
Tan, Y.-C. et al. Sequencing antibody repertoires provides evidence for original antigenic sin shaping the antibody response to influenza vaccination. Clin Immunol 151, 55-65 (2014).
Volpe, J. M., Cowell, L. G. & Kepler, T. B. SoDA: implementation of a 3D alignment algorithm for inference of antigen receptor recombinations. Bioinformatics 22, 438-444 (2006).
Giudicelli, V. IMGT/LIGM-DB, the IMGT(R) comprehensive database of immunoglobulin and T cell receptor nucleotide sequences. Nucleic Acids Research 34, D781-D784 (2006).
Schwenk, R. et al. IgG2 antibodies against a clinical grade Plasmodium falciparum CSP vaccine antigen associate with protection against transgenic sporozoite challenge in mice. PLoS One 9, e111020 (2014).
Li, K., Horn, G. Q., Alam, S. M., Tomaras, G. D. & Dennison, S. M. Titration analysis: A tool for high-throughput analysis of binding kinetics data for multiple label-free platforms. Biophysical Journal 120, 265a-266a (2021).
Bruña-Romero, O. et al. Detection of malaria liver-stages in mice infected through the bite of a single Anopheles mosquito using a highly sensitive real-time PCR. International Journal for Parasitology 31, 1499-1502 (2001).
Liu, Y. et al. High-throughput screening for developability during early-stage antibody discovery using self-interaction nanoparticle spectroscopy. MAbs 6, 483-492 (2014).
Kohli, N. et al. A novel screening method to assess developability of antibody-like molecules. MAbs 7, 752-758 (2015).
Honegger, A. & Plückthun, A. Yet another numbering scheme for immunoglobulin variable domains: an automatic modeling and analysis tool. J Mol Biol 309, 657-670 (2001).
Rogers, R. S. et al. A view on the importance of “Multi-Attribute Method” for measuring purity of biopharmaceuticals and improving overall control strategy. AAPS J 20, 7 (2017).
Ong, E.-C., Smidt, P. & McGrew, J. T. Limiting the metabolic burden of recombinant protein expression during selection yields pools with higher expression levels. Biotechnol Prog 35, e2839 (2019).
Bodwell, J., Swiff, F. & Richardson, J. Long duration electroporation for achieving high level expression of glucocorticoid receptors in mammalian cell lines. J Steroid Biochem Mol Biol 68, 77-82 (1999).

1. A recombinant anti-circumsporozoite (CSP) antibody that binds to a first epitope present in the central repeat region of CSP and binds to a second epitope of CSP, wherein the first epitope comprises an amino acid sequence selected from SEQ ID NOs: 923-974.

2.-3. (canceled)

4. The recombinant antibody of claim 1, wherein;
(i) the second epitope is heterologous to epitopes present in the RTS,S vaccine,
(ii) the second epitope comprises a minor repeat region of CSP and/or a junctional region of CSP,
(iii) the second epitope comprises a DPNA (SEQ ID NO: 977)/NPNV (SEQ ID NO: 980)-containing minor-repeat amino acid sequence and/or a DPNA (SEQ ID NO: 977)/NPNV (SEQ ID NO:980)-containing junctional amino acid sequence, or
(iv) the second epitope consists of an amino acid sequence selected from SEQ ID NOs: 975-1195.

5.-7. (canceled)

8. The recombinant antibody of claim 1, wherein the antibody binds to at least one additional epitope of CSP, and wherein:
(i) the at least one additional epitope comprises a DPNA (SEQ ID NO: 977/NPNV (SEQ ID NO:980)-containing minor-repeat amino acid sequence and/or a DPNA (SEQ ID NO: 977)/NPNV (SEQ ID NO:980)-containing junctional amino acid sequence, or
(ii) the at least one additional epitope consists of an amino acid sequence selected from SEQ ID NOs: 975-1195.

9.-14. (canceled)

15. The recombinant antibody of claim 1 comprising
a VH comprising an amino acid sequence that is at least 80% identical to an amino acid sequence selected from SEQ ID NOs: 1-461, and/or
a VL comprising an amino acid sequence that is at least 80% identical to an amino acid sequence selected from SEQ ID NOs: 462-922.

16. (canceled)

17. The recombinant antibody of claim 1 comprising a heavy chain variable region (VH) and a comprising a light chain variable region (VH), wherein
the heavy chain variable region comprises a CDR1, a CDR2, and a CDR3 of a heavy chain variable sequence set forth in any one of SEQ ID NOs: 1-461, and
the light chain variable region comprises a CDR1, a CDR2, and a CDR3 of a light chain variable sequence set forth in any one of SEQ ID NOs: 462-922.

18. (canceled)

19. The recombinant antibody of claim 1, wherein:
(i) the antibody exhibits at least 20% reduction in parasite liver load as compared to a reference antibody,
(ii) the antibody exhibits at least 20% increase in survival rate as compared to a reference antibody,
(iii) wherein the antibody exhibits increased conformational stability as compared to a reference antibody, or
(iv) the antibody exhibits increased colloidal stability as compared to a reference antibody;
and wherein the reference antibody is AB-000317, AB-000224, or AB-007088.

20.-23. (canceled)

24. A polynucleotide encoding an antibody of any one of claim 1.

25. An expression vector comprising the polynucleotide of claim 1.

26. A host cell comprising the polynucleotide of claim 24.

27. A composition comprising the antibody of claim 1.

28. (canceled)

29. A method of preventing or treating malaria in a subject in need thereof, comprising administering an effective amount of the antibody of claim 1.

30. A method of preventing or treating malaria in a subject in need thereof, comprising administering an effective amount of the composition of claim 27.

31.-35. (canceled)

36. A method of selecting an antibody as an anti-malaria therapeutic antibody, the method comprising:
a) analyzing the antibody for binding to a first epitope of the central repeat region of CSP; and
b) analyzing the antibody for binding to a second epitope of CSP that is heterologous to epitopes present in the RTS,S vaccine;
wherein the antibody is selected if it binds to both the first epitope and the second epitope.

37. The method of claim 36, further comprising:
c) analyzing the antibody for binding to at least one additional epitope of CSP that is heterologous to epitopes present in the RTS,S vaccine;
wherein the antibody is selected if it binds to the first epitope, the second epitope, and the at least one additional epitope.

38.-40. (canceled)

41. The method of claim 36, wherein the first epitope consists of the amino acid sequence selected from SEQ ID NOs: 923-974.

42. The method of claim 36, wherein;
(i) the second epitope is heterologous to epitopes present in the RTS,S vaccine,
(ii) the second epitope comprises a minor repeat region of CSP and/or a junctional region of CSP,
(iii) the second epitope comprises a DPNA (SEQ ID NO: 977)/NPNV (SEQ ID NO: 980)-containing minor-repeat amino acid sequence and/or a DPNA (SEQ ID NO: 977)/NPNV (SEQ ID NO:980)-containing junctional amino acid sequence, or
(iv) the second epitope consists of an amino acid sequence selected from SEQ ID NOs: 975-1195.

43.-45. (canceled)

46. The method of claim 36, wherein the first epitope consists of an amino acid sequence selected from SEQ ID NOs: 923-974 and the second epitope consists of an amino acid sequence selected from SEQ ID NOs: 975-1195.

47. The method of claim 36, wherein:
(i) the antibody binds to the first epitope with a binding affinity (K_{D}) that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M and/or
(ii) the antibody binds to the second epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.

48.-49. (canceled)

50. The method of claim 37, wherein
a) the first epitope consists of an amino acid sequence selected from the group consisting of SEQ ID NOs: 923-974;
b) the second epitope consists of an amino acid sequence selected from the group consisting of SEQ ID NOs: 975-1195; and
c) the at least one additional epitope of CSP that is heterologous to epitopes present in the RTS,S vaccine consists of an amino acid sequence selected from the group consisting of SEQ ID NOs: 975-1195.

51. (canceled)

52. The method of claim 37, wherein the antibody binds
a) to the first epitope with K_{D} that is less than about 106 M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M;
b) to the second epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M; and
c) to the at least one additional epitope with K_{D} that is less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.


SEQUENCE LISTING (1203 sequences)

SEQ ID NO: 1 - protein, 124 aa, synthetic construct
QVQLVESGGGVVQPGRSLRLSCAASGFTFSSYGMHWVRQAPGKGLEWVAVIWYDGNNKYYVDSVKGRFTISRDNSKNTLYLQMNSLRAEDTAVYYCARDHDADYVEKWGAFDLWGQGTMVTVSS

SEQ ID NO: 2 - protein, 122 aa, synthetic construct
QVQLVESGGGVVQPGRSLRLSCAASGFTFNSYGMHWVRQAPGKGLEWVALISFDGTNKFYADSVKGRFTISRDNSKNTLYLQMNSLRAEDTALYHCATDSLFYYDFHLFDYWGQGTLVTVSS

SEQ ID NO: 3 - protein, 127 aa, synthetic construct
QVQLVESGGGVVQPGRSLRLSCAASGFTFSSYGMHWVRQAPGKGLEWVAIIWYDGSHKYYADSVKGRFTISRDNSKNTLYLQMNSLRAEDTAVYHCARVYQPTYYYDSSRGEAFDIWGQGTMVTVSS

SEQ ID NO: 4 - protein, 122 aa, synthetic construct
QVQLVESGGGVVQPGRSLRLSCAASGFSFSTYGMHWVRQAPGKGLEWVAIIWYDGSKEFYADSVKGRFTISRDNSKNTLYLQMNSLRADDTAVYYCVKDDFDSSGHSYFQHWGPGTLVTVSS

SEQ ID NO: 5 - protein, 122 aa, synthetic construct
EVQMVESGGRLIKPGGSLRLTCTTSGFSFTNAWMTWVRQAPGKGPEWVGRIQSESDGGTRDYAAPVKGRFIISRDDSLNTLYLQMNSLKTEDTAVYYCITDRDFYGSTSPSWGQGTLVIVSS

SEQ ID NO: 6 - protein, 126 aa, synthetic construct
QVQLVESGGGVVQPGRSLRLSCAASGFKFISYGMHWVRQAPGKGLEWVAVIWHDGSIRFYADSVKGRFTISRDNSNNILYLQMSSLRAEDTAVYYCATSGYCRGGSCDGGVYFDSWGLGTLVTVSS

SEQ ID NO: 7 - protein, 126 aa, synthetic construct
QVQLVESGGGVVQPGRSLRLSCAASGFTFSTYGMHWVRQAPGKGLEWVAVIWYDGSNKYYADSVKGRFTISRDNSKNTLYLQMNSLRAEDTAVYYCARVRQEPYYYASRGSALDIWGQGTMVTVSS

SEQ ID NO: 8 - protein, 125 aa, synthetic construct
QVQLVQSGAEVKKPGASVKVSCKASGYTFTSSEINWVRQATGQGLEWVGWMNPNTHKTIYAQKLQGRVTMTSDTSKSTAYMELSSLRSEDTAVYYCARGGYCIHNNCYDVQLDPWGQGTLVIVSS

SEQ ID NO: 9 - protein, 123 aa, synthetic construct
QVQLVEFGGGVVQPGRSLRLSCSASGFSFRSYGMHWVRQAPGKGLEWVAVIWYDGSNYYYTKSVEGRFTISRDNSKNMLFLQMNSLRADDTAVYYCARPENSDYDRGGAFDIWGQGTVVTVSS

SEQ ID NO: 10 - protein, 119 aa, synthetic construct
QMQLVESGGGVVQPGRSLRLSCAASGFTFSDYAMHWVRQSPGQGLEWVAVISYHSRNKYYPESVKGRFTISRDNSKNTLYLQINSLRTEDTAVYYCARDGYSSSFFDFWGQGTLVTVSS

SEQ ID NO: 11 - protein, 122 aa, synthetic construct
GVQLVESGGGLVQPGRSLRLSCTVSGFTFGDYAMSWVRQAPGKGLEWVGFIRSEANGGKTEYAASVKGRFTISRDDSKSIAYLQMRSLTTEDTAVYYCTRVMLGASWGLDFWGQGSLVTVSS

SEQ ID NO: 12 - protein, 122 aa, synthetic construct
EVQLVQSGGGLVQPGGSLRLSCVASGFTLSSYEMNWVRQAPGKGLEWVSFISSSGSKKRYADSVKGRFTVSRDNAKNSLSLQMNSLRAEDTAVYYCARDHSTSDREYGLDVWGQGATVTVSS

SEQ ID NO: 13 - protein, 122 aa, synthetic construct
EVQLVESGGGLVQPGRSLRVSCATSGFPFGDHAMSWFRQAPGKGLEWVGFIRSKTYGGAAEYAASVKGRFTISRDDSRSIAHLQMNSLKTEDTGVYYCSRVRTYDFRDMDVWGKGTTVTVSS

SEQ ID NO: 14 - protein, 123 aa, synthetic construct
QVQLVESGGGVVQPGRSLRLSCVASGFSFRSYGMHWVRQAPGKGLEWVAIIWYDGSNKYYAHSVKGRFTISRDNSKNTLYLQMNSLRAEDTAIYYCAKDGGTETTWGNAFDIWGQGTMVTVSS

SEQ ID NO: 15 - protein, 125 aa, synthetic construct
QVQLVESGGGVVQPGRSLRLSCAASGFTFSSYGMHWVRQAPGKGLEWVAVIWYDGSNKYYADSVKGRFTISRDNSQNTLFLQMNSLRAEDTAVYYCARVGEYSSSPDWGAAFDIWGQGTMVTVSS

SEQ ID NO: 16 - protein, 122 aa, synthetic construct
QVQLVESGGGVVQPGRSLRLSCAASGFTFSNYGMHWVRQAPGKGLEWVAIIWYDGSSKYYADSVRGRFTISRDNSKNTLFLQMNSLRAEDTAVYYCARDDYYSSAYNHLGYWGQGTLVTVSS

SEQ ID NO: 17 - protein, 123 aa, synthetic construct
QVQLVESGGGMVQPGRSLRLSCAASGFSFSSYGMHWVRQAPGKGLEWVGVIWYDGGYKLYADSVRGRFTISRDNSKNTLYLQMNSLRVEDTAVYYCARPGYCDSTNCYAFDYWGQGTLVTVSS

SEQ ID NO: 18 - protein, 124 aa, synthetic construct
QIQLVESGGGVVQPGRSLRLSCVTSGFTFSNFGMHWVRQAPGKGLEWVALIWHDGSKKYYADSVEGRFTISRDNSKKTLYLQMNSLRAEDTAVYYCARDGGSPDGSVYNYFEYWGQGTLVTVSS

SEQ ID NO: 19 - protein, 126 aa, synthetic construct
QVQLVESGGGVVQPGRSLRLSCAASRFTFKNYGMHWVRQGPGKGLEWVAVIWYDGSQEYYVDSVRGRFTISRDNSKDTLYLQMNSLRAEDTAVYYCVRDGRYRTSHQTSTGFFDLWGQGTLVTVSS

SEQ ID NO: 20 - protein, 124 aa, synthetic construct
QVQLVESGGGVVQPGRSLRLSCAASGFTFSNYGMHWVRQAPGKGLEWVAVIWYDGSNKFYADSVKGRFTSSRDNSKNTLYLQMNSLRAEDTAVYYCARDGRFLTTVTTAYFDYWGQGTLVTVSS

SEQ ID NO: 21 - protein, 124 aa, synthetic construct
QVQLVESGGGVVQPGRSLRLSCAASGFTFSSYGMHWVRQAPGKGLEWVAVIWYDGSNKYHADSVKGRFTISRDNSKNTLYLQMNNLRSEDTAVYYCAKDETLWFGELSDGFDIWGQGTMVTVSS

SEQ ID NO: 22 - protein, 121 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDLDYSNHNWFDPWGQGTLVTVSS

SEQ ID NO: 23 - protein, 122 aa, synthetic construct
QVQLVESGGGVVQPGRSLRLSCAASGFTFRDYGMHWVRQVPGKGLEWVAIIWHDGSKKYYADSVKGRFTISRDNFKNTLYLQMNSLRAEDTAVYYCAKVFDHAESSYTFDIWGQGTMVTVSS

SEQ ID NO: 24 - protein, 124 aa, synthetic construct
QVQLVESGGGVVQPGRSLRLSCAASGFPFSSYGMHWLRQAPGKGLEWVAVIWYDGSNYYYADSVKRRFTISRDNSKNTLYLQMNSLRAEDTAVYYCARVGMGDYLDSSGYYDYWGQGTLVTVSS

SEQ ID NO: 25 - protein, 113 aa, synthetic construct
EVQLVESGGGLVQPGGSLRLSCAASGFTFNNYWMSWVRQTPGMGLEWVANINQDGSKKDYVDSVEGRFTISRDNAKNSLYLLMNSLRADDTAVYYCARYLNAWGQGILVTVSS

SEQ ID NO: 26 - protein, 123 aa, synthetic construct
QVQLVESGGGVVQPGRSLRLSCAASGFTFSFYGMHWVRQAPGKGLEWVAVIWYDGSNKYYADSVRGRFTISRDNSKNTLYLQMNSLRAEDTAVYYCARVGNDYDDHGSALDMWGQGTMVTVSS

SEQ ID NO: 27 - protein, 123 aa, synthetic construct
QVQLVESGGGVVQPGRSLRLSCVVSGFTFSSYGMHWVRQAPGKGLEWVAIIWYDGSKKYYADSVKGRFTISRDNSKNTLYLQMNSLRAEDTAVYYCARPGDSSGSHGEAFDIWGQGTMVTVSS

SEQ ID NO: 28 - protein, 124 aa, synthetic construct
QVQLVESGGGVVQPGRSLRLSCAASGFTFSSYGMHWVRQAPGKGLEWVAVIWYDGSYKYYADSVKGRFAVSRDNSKNTLYLQMNSLRTEDTAVYYCAKEGRTVEVAATGWFDPWGQGTLVTVSS

SEQ ID NO: 29 - protein, 120 aa, synthetic construct
QVLLVESGGGVVQPGGSLRLSCTASGFTFKNYGMHWVRQAPGKGLEWVAVIWFDGSNEFYADSVEGRFTISRDNSKNTLYLQTNSLRAEDTAVYYCARSYTSSWLGFDFWGQGTLVTVSS

SEQ ID NO: 30 - protein, 121 aa, synthetic construct
EVQLVESGGGLVQPGRSLRLSCTASGFTFGDYAMSWFRQAPGKGLEWVSFIRSKTYGGSTEYAASVKGRFTISRDDSKSIAYLQMHSLKTEDTAFYYCTRVSAAAGVGDYWGQGTLVTVSS

SEQ ID NO: 31 - protein, 122 aa, synthetic construct
QVQLVESGGGVVQPGRSLRLSCAASGFTFSSYGMHWVRQAPGKGLEWVAIIWYDGSNKYYADSVKGRFTISRDNSKNTLYLQMNSLRAEDTAVYYCARDTDDYYDAHTMDVWGQGTTVTVSS

SEQ ID NO: 32 - protein, 124 aa, synthetic construct
QVQLVESGGGVVQPGRSLRLSCAASGFTFSSYGMHWVRQAPGKGLEWVAIIWYDGSNKYYADSVKGRFTISRDKSKNTLYLQMNSLRAEDTAVYYCAKVSDSSDYSHSGAFDIWGQGTMVTVSS

SEQ ID NO: 33 - protein, 121 aa, synthetic construct
EVQLVESGGGLVQPGRSLRLSCTASGFTFGDYAMSWFRQAPGKGLEWVGFIRSKANGGTGEYAASVKGRFTISRDDSKNIAYLQMDSLKTEDTAVYYCTRVLVVVAAGDYWGQGTLVTVSS

SEQ ID NO: 34 - protein, 122 aa, synthetic construct
QVQLVESGGGVVQPGRSLRLSCTASGFTFSNYGMHWVRQAPGRALEWVAIIWHDGSNKYYADSVKGRFTISRDNSKNTLYLQMNNVRVEDTAVYHCAKVGGFGELYEAFDFWGQGTLVTVSS

SEQ ID NO: 35 - protein, 122 aa, synthetic construct
QAQPVESGGGVVQPGKSLRLSCAASGFTFRNFGMHWVRQAPGKGLEWVAVIWHDGSNQFYADSVEGRFTISRDNSKNTLYLQMDSLRVEDTAMYYCARDRGYSGSWGYFDHWGQGTLVTVSS

SEQ ID NO: 36 - protein, 124 aa, synthetic construct
QVQLVESGGGVVQPGRSLRLSCAASGFTFSNFGMHWVRQAPGKGLEWVAVIWYDGSKKYYADFVKGRFTISRDNSKYMLYLQMTSLGAEDTAIYYCARDIRGAYRVTDGGFDYWGQGALVTVSS

SEQ ID NO: 37 - protein, 123 aa, synthetic construct
QVQLVESGGGVVQPGRSLRLSCAASGFTFRNNGMHWVRQAPGKGLEWVAVIWHDGSNKYYADAVKDRFTISRDNSKNTLYLQMNSLRAEDTAVYYCVKVAFCTGDCYPPMDVWGKGTTVTVSS

SEQ ID NO: 38 - protein, 118 aa, synthetic construct
QAQLVESGGGVVQPGRSLRLSCAASGFTFSSYGMHWVRQAPGKGLEWVAVIWHDGSNQYYADSVKGRFTISRDNSKNTLYLQMNSLRAEDTAVYYCAKVGDLLALDYWGQGTLVTVSS

SEQ ID NO: 39 - protein, 125 aa, synthetic construct
QVQLVESGGGVVQPGRSLRLSCAASGFMFKNYGMHWVRQAPGEGLEWVTVIWHDGSNEYYADSVKGRFTVSRDNSKNTLYLQMNSLRAEDTAVYYCARVGDGTTKYDSSGFLDCWGQGTLVTVSS

SEQ ID NO: 40 - protein, 124 aa, synthetic construct
QVQLVESGGGVVQPGRSLRLSCAASGFSFSNYGMHWVRQAPGKGLEWVAIIWYDGSNKYYADSVKGRFTISRDNSKNTLYLQMNSLRAEDTAVYYCARDGTIWDDSRGYILDYWGQGTLVTVSS

SEQ ID NO: 41 - protein, 121 aa, synthetic construct
QVQLVESGGGVVQPGRSLRLSCAASGFTFSNYGLHWVRQAPGKGLEWVALIRYDGNNKFYADSLKGRFTISRDNSKDTLYLQMNSLRVEDTAVYYCAKVGSGNYGNYMDVWGKGTTVTVST

SEQ ID NO: 42 - protein, 121 aa, synthetic construct
QVQLVESGGGVVQPGRSLRLSCAASRFPFKNYGMHWVRQAPGKGLEWVALIWFDGSNKFYVDSVEGRFTISRDNSNNMLYLEMNSLRADDTAVYYCARAGRYDDSGYLDYWGQGTLVTVSS

SEQ ID NO: 43 - protein, 120 aa, synthetic construct
QVQLVESGGGVVQPGRSLRLSCAASGFTFSSYGMHWVRQAPGKGLEWVAVIWYDGSNKFYADSVKGRFTVSRDNSKNTLYLQMNSLRAEDTTVYYCARDGGVTTRYFDLWGRGTLVTVSS

SEQ ID NO: 44 - protein, 123 aa, synthetic construct
QEQLVESGGGVVQPGRSLRLSCAASGFTFSTYAIHWVRQAPGKGLEWVAVISYDGSSTWFSNSVEGRFTFSRDNSKNTVFLQMNSLSAEDTAVYYCARDGNDYPAYYYYMDVWGKGTTVTVSS

SEQ ID NO: 45 - protein, 125 aa, synthetic construct
QVQLVESGGGVVQPGRSLRLSCAASGFTFSTYGMHWVRQAPGKGPEWVAVIWYDGSNKYYRDSVKGRFSISRDNSKNTLYLQMNSLRAEDTAVYYCAKDGRPLKGGVTTGWFDPWGQGTLVTVSS

SEQ ID NO: 46 - protein, 127 aa, synthetic construct
QVQLVESGGGVVQPGRSLRLSCAASGFSFSSYGMHWVRQAPGKGLEWVAVIWYDGSNKYYADSVKGRFTISRDNSKNTLYLQMNSLRAEDTAVYYCATAAQGYCSGGSCYGHTFDYWGQGTLVTVSS

SEQ ID NO: 47 - protein, 122 aa, synthetic construct
QVQLVESGGGVVQPGRSLRLSCAASGFTFSSYGMHWVRQAPGKGLEWVAVIWYDGSKKYYADSVKGRFTISRDNSKNTVYLHMNSLRVEDTAVYYCARPEAVAGTGGWFDHWGQGNRVTVSS

SEQ ID NO: 48 - protein, 121 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHYSDTSDYYYDYWGQGTLVTVSS

SEQ ID NO: 49 - protein, 121 aa, synthetic construct
QVQLVESGGGVVEPGGSLRLSCAASGFTFSNYGMHWVRQAPGKGPEWVAVIWHDGSKKYYEDSVEGRFTISRDNSKNTLYLKMNSLRVEDTAVYYCAKDRGGDRGNFGHYWGQGTLVTVSS

SEQ ID NO: 50 - protein, 123 aa, synthetic construct
EVQLVESGGGLVQPGRSLRLSCTASGFTFGDYAMSWVRQAPGKGLEWVGFIRRKAHGGTTEYAASVKGRFTISRDDSKSIAYLQMNSLKTEDTAVYYCTRVEPTVVTPVFDYWGQGTLVTVSS

SEQ ID NO: 51 - protein, 130 aa, synthetic construct
QEQLVESGGAVVQPGGSLRLACVGTGFSFSSYGIHWVRQAPGKGLEWVALISYDGSNKYYGESVRGRFTISRDNSKDTVYLQMKSLRGDDTAVYYCATAYYNFWHSYYMLPTKVLGMDVWGQGTTVTVSS

SEQ ID NO: 52 - protein, 126 aa, synthetic construct
EVQLVQSGAEVRKPGTTVKISCDVSGYTFTDYYIYWVQQAPGKGLEWMGLVDLENGETKYAEKFQGRVTITADTSTDIAHMELSSLRSEDTAIYYCATDSSCGDECNVPGGAFDIWGQGTIITVSS

SEQ ID NO: 53 - protein, 121 aa, synthetic construct
QVHLVQSGAEVKKPGASVKVSCKTSGYSFTGYYMHWVRQAPGQGLEWMGWINPHSGGTKDAPKFQGRVTLTRDTSINTSYMELDSLTSDDTAVYFCARSEQVVLLGGFDFWGQGTLVTVSS

SEQ ID NO: 54 - protein, 122 aa, synthetic construct
EVQLVESGGGLVQPGRSLRLSCTTSGFSFGDYAMNWVRQAPGKGLEWVGFIRSNAYGGTAEYAASVKGRFTISRDDPNSIAYLQMNSLRTEDTAVYYCTRLEAGVKRGMDVWGQGTTVTVSS

SEQ ID NO: 55 - protein, 122 aa, synthetic construct
QVQLVESGGGVVQPGKSLRLSCIASGFPFSTYGMHWVRQAPGKGLEWVAIIWHDGSKEFYGDSVKGRFTISRDNSKNTLYLQMNSLTAEDTAMYYCVKDDFDSSGYSYFQHWGQGTLVTVSS

SEQ ID NO: 56 - protein, 123 aa, synthetic construct
QVQLVESGGGVVQPGRSLRLSCAASGFAFNTYGMHWVRQTPGKGLEWVAIIWYDGSQKYYADSVQGRFIISRDNHKNTLSLQMNGLRAEDTAVYFCVRVRFSVGPHGSAFDLWGQGTMVIVSS

SEQ ID NO: 57 - protein, 122 aa, synthetic construct
QVQLVESGGGVVQPGRSLRLSCAASGFTFSSFGMHWVRQAPGKGLEWVAIIWYDGNNKYYADSVKGRFTISRDNSKNTLYLQMNSLRDDDTAVYYCAKDDYDSGGYNQFESWGQGTLVIVSS

SEQ ID NO: 58 - protein, 127 aa, synthetic construct
EVQLVESGGGLVQPGRSLRLSCKASGFNFGDHAVSWVRQAPGKGLEWVGFIRSKVYGGTTKYAASVKGSFTISRDDSKSISYLEMNSLKTEDTAVYYCTRVQLDYGPGYYYYGMDVWGQGTKVTVSS

SEQ ID NO: 59 - protein, 121 aa, synthetic construct
QVQLVESGGGVVQPGRSLRLSCAASGFTFKNRGMHWVRQAPGKGLEWVAVIWHDGSKEFYADSVEGRFTVSRDNSKNTLYLQMNSLRAEDTAIYYCVRAAYYDTSGYGDYWGQGALVTVSS

SEQ ID NO: 60 - protein, 121 aa, synthetic construct
QVQLVESGGGMVQPGRSLRLSCVASQFTFNNYGMHWVRQAPGRGLEWLALIWYDGSNKFYADSVEGRFTISRDNSKNTLYLQMSSLRADDTAVYYCARAGKYDNSGYLDYWGQGTLVTVSS

SEQ ID NO: 61 - protein, 121 aa, synthetic construct
QMQLVESGGGVVQPGRSLRLSCAASGFSFNTYGMHWVRQAPGKGLEWVAVIWHDGSKKFYRDSVEGRFTVSRDNSKDTLYLQMNSLRVEDTAVYYCARDFSYSSHNYFHPWGQGTLVTVSS

SEQ ID NO: 62 - protein, 121 aa, synthetic construct
EVQLVESGGGLVQPGRSLRLSCATSGFIFGDYAMSWFRQAPGKGLEWVGFIRSKGYGGSTEYAASVKGRFTISRDDSKSVAYLQMNSLKTEDTAVYYCSKIGIVVPSAVYWGQGTLVTVSS

SEQ ID NO: 63 - protein, 122 aa, synthetic construct
QVQLVESGGGVVQPGRSLRLSCAASGFTFNNYGMHWVRQAPGKGLQWVALISYDGSNKFYADSVEGRFTISRDSADNTLYLQMDSLRPEDTAVYYCATDSLNYYDFHRFDNWGQGTLVIVSS

SEQ ID NO: 64 - protein, 121 aa, synthetic construct
QVQLVESGGGVVQPGRSLRLSCAASGFTFYRYGMHWVRQAPGKGLEWVAVLWHDGSNKYYADSLKGRFTISRDNSKSTLYLQMNSLTAEDTAVYYCARAAYHDTSGYGDYWGQGTLVTVSS

SEQ ID NO: 65 - protein, 124 aa, synthetic construct
QLQLVESGGGVVQPGRSLRLSCAASGFTFSSYGMHWVRQAPGKGLEWVAVIWHDGSTKYHAESVEGRFTISRDNSRNTLYLQMNSLRAEDTAVYYCARVGMGGQYDSSGYYDYWGQGTLVTVSS

SEQ ID NO: 66 - protein, 123 aa, synthetic construct
QVQLVESGGGAVQPGRSLRLSCVASGFGFRNYGMHWIRQAPGRGLEWVAIIWYDGSQKYYSDSVEGRFTISRDNSKNTLYLQMNSLRVEDTAIYYCVRVRYSSSRHGSAFDLWGQGTTVIVSS

SEQ ID NO: 67 - protein, 124 aa, synthetic construct
QVHLLESGGTVVQPGGSLRLSCEASGFIFSDYGMHWVRQVPGKGLEWVAVIWFDGSQKYYGDSVKGRFTVSRDNSKNTLYLQMNTLRADDTGIYYCATDIRGAYKVTDGGFDQWGHGTLVTVSP

SEQ ID NO: 68 - protein, 123 aa, synthetic construct
QAQLVESGGGVVQPGRSLRLSCAASGFTFRNYGMHWVRQAPGKGLDWVAIIWYDGSQTYYADSVQGRFTISRDNSKDTLYLQMNNLRAEDTAVYYCARVRYSSSQHGSAFDLWGQGTMVTVSS

SEQ ID NO: 69 - protein, 124 aa, synthetic construct
QVQLVESGGGVVQPGRSRRLSCAASGFYFNTYGMHWVRQAPGKGLEWVALIWFDGSKKFYADSVKGRFTISRDNSKDTLYLQMNSLRAEDTAVYYCARDGAAYDDSNGGAFDIWGQGTLVTVSS

SEQ ID NO: 70 - protein, 122 aa, synthetic construct
QVQLVESGGGVVQPGRSLRLSCAASGLIFRSSGMHWVRQAPGKGLEWVSLISYDGSNKFYADSVKGRFTISRDNSKNTLYLQMNSLRADDTAVYYCATDSLSYYDYHLFDYWGQGALVTVSS

SEQ ID NO: 71 - protein, 127 aa, synthetic construct
EVQLVESGGGLVQPGRSLRLSCIASGFTFGDHAMSWVRQAPGRGLEWLGFIRSKTYGGTPKYAASVKGRITISRDDSKSMVYLQMNSLKTDDTAVYYCTRVQLDQGPGYYYYGMDVWGQGTTVTVSS

SEQ ID NO: 72 - protein, 121 aa, synthetic construct
QEQLVESGGGVVQPGGSLRLSCAASGFSFGSYGMHWVRQAPGKGLEWVAVIWYDGSQKFHADSLKGRFTISRDNVKNTLYLQMNSLRGEDTAVYYCARDLDYYNHNYFDPWGQGTLVTVSS

SEQ ID NO: 73 - protein, 122 aa, synthetic construct
QVQLVESGGGVVQPGRSLRLSCAASGFTFRNSGMHWVRQAPGKGLEWVALITHDGSNEFYADSVKGRFTISRDNSKNTLFLQMNSLRADDTAMYYCATDSLHYFDYHLFDYWGQGTLVTVSS

SEQ ID NO: 74 - protein, 125 aa, synthetic construct
QVYLVESGGGVVQPGRSLRLSCAASGFIFRSYGMHWVRQAPGKALEWVAVIWYDGSNKFYSDSVKGRFTISRDNSKNTLYLEMNSLRAEDTAVYYCARVGDGTTRFDSSGFLDFWGQGTLITVSS

SEQ ID NO: 75 - protein, 121 aa, synthetic construct
QVQLVESGGGVVQPGRSLRLSCAASGFIFKNSGMHWVRQVPGQGLEWLALIWFDGSNEFYADSVEGRFTISRDNSKNTLYLQMNSLRVEDTAVYYCARAGQFDNSGYLDYWGQGALVIVSS

SEQ ID NO: 76 - protein, 124 aa, synthetic construct
QVQLVESGGGLVQPGRSLRLSCEASGFIFSDCGMHWVRQTPGKGLEWVSVIWFDGSKQYYADSVKGRFTISRDNSKNMLYLQMNSLSTEDSAVYYCARDIRGAYQVADGGFDCWGQGTLVTVSS

SEQ ID NO: 77 - protein, 121 aa, synthetic construct
DMQVVESGGGLVKPGGSLRLSCAVSGFSVNNAWMSWVRQVPGKGLEWVGRIRSKTYGGTTDYAAPVKDRFTISRDDSKNTLYLQMNSLKSEDTGVYYCITDRDFYRSGGYWGQGTLVTVSS

SEQ ID NO: 78 - protein, 121 aa, synthetic construct
QVQVVESGGGVVQPGRSLRLSCAASGFIFSNYGMHWVRQAPGKGLEWVAVIFYDGSKTYYADSAKGRFIISRDNSKNTLYLQMNSLRAEDTAVYYCARAGYSDSSGYGDYWGQGTLVTVSP

SEQ ID NO: 79 - protein, 121 aa, synthetic construct
QVHLVESGGGVVQPGRSLRLSCAASGFTFRNYGIHWVRQAPGKGLEWVAVIWYDGSREFYADSVEGRFTVSRDNSKNTVFLQMNSLRVDDTAVYYCARAGYSDTSGYGDHWGQGTLVSVSS

SEQ ID NO: 80 - protein, 127 aa, synthetic construct
EVRLEESGGGLLQPGRSLRLSCTASGFRFGDYAMSWVRQAPGKGLEWVGFIRSKTFGGSTRNAASLRGRFTLSRDDSKSVAYLQMNSLTTEDTAVYYCARVQLDSGPGYYYYGMDVWGQGTTVIVSS

SEQ ID NO: 81 - protein, 121 aa, synthetic construct
QEHLVESGGGVVQPGKSLRLSCAASGFTFSNYGMHWVRQAPGRGLQWVAVIWYDGSQKFYAEFARGRFTVSRDNSKNTLYLQMNSLRADDTAVYYCTRDYDYSSHNYFDPWGQGTLVTVSS

SEQ ID NO: 82 - protein, 122 aa, synthetic construct
QVHLVESGGGVVQPGRSLTVSCAASGFTFRNYGMHWVRQVPGKGLEWVGVIWHDGSQEHYGESVEGRFSISRDNSKNTLYLQMNSLGAEDTAVYHCARDRGYTGSWGYFDYWGQGTLVTVSS

SEQ ID NO: 83 - protein, 122 aa, synthetic construct
QVQLVESGGGVVQPGRSLRLSCAASGFTFSYYGMHWVRQAPGKGLEWVAIIWYDGSNKYYADSVKGRFTISRDNSKNTLYLQMNSLRAEDTAVYYCARDDFDSGFYNHLAYWGQGTLVTVSS

SEQ ID NO: 84 - protein, 122 aa, synthetic construct
QVQLAESGGGVVQPGRSLRLSCAASGFIFRNYGMYWVRQAPGKGLEWLAVIWHDGSNEFYADSVEGRFTISRDNSKNTLFLHMSSLRVEDTAVYYCVRDRGYTDSWGYFGFWGQGTLVTVSS

SEQ ID NO: 85 - protein, 127 aa, synthetic construct
QVQLVESGGGVVQPGRSLRLSCAASGFTFGSYGMHWVRQAPGKGLEWVAIIWYDGSNKYYADSVKGRFTISRDNSKNTLYLQMNSLRAEDTAVYYCARVYQPTDYYDSSRGDAFDIWGQGTMVTVSS

SEQ ID NO: 86 - protein, 125 aa, synthetic construct
QVQLVESGGGVVQPGRSLRLSCAASGFTFRSYGMHWVRQAPGKGLEWVAVIWYDGSNKYYPDSVKGRFTISRDNSQNTLFLQMNSLRAEDTAVYYCARVGEYSSSPDWGAAFDIWGQGTMVIVSS

SEQ ID NO: 87 - protein, 127 aa, synthetic construct
EVQLVESGGGLGQPGRSLRLSCTASGFTFGDYAMSWVRQAPGKGLEWLGFIRSKTYGGSTKYAAPVKARVIITRDDSKSIAYLQMNSLKTEDTAVYYCARVQLDSGPGYYYYGMDVWGQGTTVTVSS

SEQ ID NO: 88 - protein, 121 aa, synthetic construct
EVQLVESGGGLVQPGRSLRLSCTTSGFTFGDYAMSWFRQAPGKGPEWVGFIRSKGYGGTAEYAASVKGRFVISRDDAKSIAYLQMNSLKTEDTAIYYCARVGILAASAVDWGQGTLVAVSP

SEQ ID NO: 89 - protein, 121 aa, synthetic construct
QVQLVESGGGVVQPGRSLRLSCATSGFTFSNYGMHWVRQAPGKGLEWVAVTWHDGSKKFYADSVDGRFTISRDNSKNTVSLQMNSLRVEDTAVYYCARAAYYDTSGYGDYWGHGTLVTVSS

SEQ ID NO: 90 - protein, 124 aa, synthetic construct
QVQLVESGGGVVQPGRSLRLSCAATGFTFSNFGMHWVRQAPGKGLEWVAVIWHDGSQKYYGEFVKGRFTISRDNSQNTLHLQLNSLRVDDTATYFCARDIRGAYQATDGGFDSWGQGTQVTVSS

SEQ ID NO: 91 - protein, 122 aa, synthetic construct
EVQLVESGGGWVQPGRSLRLPCTTSGFSFEDYAMSWFRQAPGRGLEWVGFIRSKANGGATESAASVKGRFTISRDDPKSIAYLQMNSLKTEDTAVYYCARVRTNDFRDMDVWGKGTTVIVSS

SEQ ID NO: 92 - protein, 113 aa, synthetic construct
ELQLVESGGGLVQPGGSLRLSCAASGFTFSNYWMSWVRQAPGKGLEWVANINQDETIKYYLDSVKGRFTVSRDNAKNLLYLQMNDLRAEDTAVYYCARYLNYWGQGILVTVSS

SEQ ID NO: 93 - protein, 121 aa, synthetic construct
EVQLVESGGGLVQPGRSLRVSCTTSGFTFGDYAMSWFRQAPGKGLEWVGFIRSKGYGGTPEYAASVKGRFTISRDDSKRTAYLQMNSLKTEDTAVYYCTRVGIEVSTAVYWGPGTLVTVSS

SEQ ID NO: 94 - protein, 127 aa, synthetic construct
EVQLVESGGGLVQPGRSLRLSCTASGFSFGDHALSWVRQAPGKGLEWVGFIRSKTYGGSTKYAASVKGRVTMSRDDAKRIAYLQINSLKTDDTAVYYCARVQLDPGPGYYYYGMDVWGQGTTVTVSS

SEQ ID NO: 95 - protein, 121 aa, synthetic construct
EVQVVESGGGLVKPGGSLRLSCAASGFTFSKAWMSWVRQAPGKGPEWVGRIESRTDGGTIEYAAAVKGRFTISRDDSKDTLYLQMNSLKTEDTAVYYCITDRDFYRSGGHWGQGTLVTVSS

SEQ ID NO: 96 - protein, 122 aa, synthetic construct
EGQLVESGGGLVQPGGSLKLSCAASGFTFSEYEMNWVRQAPGKGLEWVSYISRSGSPRHYADSVKGRFTVSRDNTKNSLFLQMNSLRVEDTAVYYCARDHSTSATEYGLDVWGQGTTVIVSS

SEQ ID NO: 97 - protein, 127 aa, synthetic construct
EVQLVESGGGLVQPGRSLRLSCTASGFIFPEYAMSWVRQAPGKGLEWLGFIRKKAYGGTTNYAASVKGRITLSRDDSKSIAYLQMNSLKIEDTAVYYCTRVQLDSGPGYYYYGMDVWGQGTTVTVSS

SEQ ID NO: 98 - protein, 127 aa, synthetic construct
EVQLVESGGGLVQPGRSLRLSCAASGFNFGDYAMSWVRQAPGKGLEWVGFIRKKAYGGTTRYAASEKGRFTISRDDSKSIAYLQMNSLKTEDTAVYYCARVQLDSGPGYYYYGMDIWGQGTTVTVSS

SEQ ID NO: 99 - protein, 122 aa, synthetic construct
EVQVVESGGGVVQPGRSLRLSCTASGFTFGDYAMSWFRQAPGKGLEWVGFIRSEANGGAPEYAASVKGRFTISRDDFKSIAYLQMNSLKTEDTAAYYCTRVRTNDFRDMDVWGKGTTVAVSS

SEQ ID NO: 100 - protein, 121 aa, synthetic construct
EVQLVESGGGLVQPGRSLRLSCTTSGFTFGDYAMSWFRQAPGKGLEWVGFIRSTGFGGSAEYAAFVKGRFTISRDDSKSIAYLQMNILKPEDTAVYLCSRVGVGISTAVNWGQGTLVTVSS

SEQ ID NO: 101 - protein, 124 aa, synthetic construct
QVQLVESGGGVVQPGKSLTLSCAASGFSFRNYGMHWVRQAPGKGLEWVAVIWFDGSKEYYADSVYGRFAISRDNSKNTLYLQMNTLRVEDTAIYYCATDIRGAYKVTDGGFGHWGQGTLVTVSP

SEQ ID NO: 102 - protein, 113 aa, synthetic construct
EMQLVESGGGLVQPGGSLRLSCAASGFTFRNYWMSWVRQAPGKGLEWVANINQDESIKYYLDSVKGRFTISRDNDKNLLFLQMSGLRAEDTSIYYCARYLNFWGQGALVTVSS

SEQ ID NO: 103 - protein, 123 aa, synthetic construct
EVQLVESGGGLVKPGGSLRLSCAASGFTFSSYTMIWVRQAPGKGLEWVSSISSSSSKIYHPNSLKGRFTVSRDNAKNSVFLHMNSLRAEDTAIYYCARTFLLAAAATGGIEYWGQGTLVTVSS

SEQ ID NO: 104 - protein, 124 aa, synthetic construct
EVQLVESGGGLVQPGGSLRLSCAGSGFTFTYYGMNWIRQAPGKGLEWVSYISVGSDIIHYADSVKGRFSISRDNAKKSLYLQMNSLRVEDSAVYFCVRGVSSGHYGTEDLLDYWGQGTLVTVSS

SEQ ID NO: 105 - protein, 117 aa, synthetic construct
QVQLVESGGGVVQPGRSLRLSCEASGFTFSDYAMHWVRQAPGKGLEWVTFMSYDGRNEYYADSVKGRFTISRDNSKNTLYLQMTSLRRDDTAVYYCARDRVGSLVYWGQGTLVTVSS

SEQ ID NO: 106 - protein, 123 aa, synthetic construct
EVLLVESGGGLVKPGGSLRLSCAASGFTFSSSTMNWVRQAPGKGLEWVASISSRSSYIYYADSLKGRFTVSRDNANNSVYLQMNSLRAEDTAIYFCARGGGMGLVTTGYFEYWGQGTLVTVSS

SEQ ID NO: 107 - protein, 124 aa, synthetic construct
EVQLVESGGGLVKPGGSLRLSCAASGFTFNTYTMNWVRQAPGKGLEWVSSISSSGSKKYYGDSVKGRFSISRDNARNSLYLQMNSLRDEDTAVYYCVRDGNVMIRGTGDWFDPWGQGTLVTVTS

SEQ ID NO: 108 - protein, 124 aa, synthetic construct
QVHLQQSGPGLVQPSETLSLTCSVSGGSISGFYWSWIRQPPGKGLEWIGFIYRSASTDYNPSLKSRVTISLDTSKNQFSLKLNSVTAADTAVYYCARDTGSYMKGWGDYGMDVWGQGTTVTVSS

SEQ ID NO: 109 - protein, 123 aa, synthetic construct
EVRLVESGGGLVKPGGSLRLSCGASGFTFNTYTMNWVRQAPGKGLEWVSSISSGSGKIYYADSVKGRFTISRDNAKNSLYLQMNGLRVEDTAIYYCAREERVGNWYSGGMDVWGQGTPVTVSS

SEQ ID NO: 110 - protein, 124 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASGVTTGWDEIHDAFDIWGQGTMVTVSS

SEQ ID NO: 111 - protein, 124 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAMYYCAKDYYYDSSGQTRLFDYWGQGTLVTVSS

SEQ ID NO: 112 - protein, 121 aa, synthetic construct
QEQLVESGGGVVQPGRSLRLSCAASGFTFSSYGMHWVRQAPGKGLEWVALIWYDGSNKYYADSVKGRFTVSRDNSKETLYLQMNSLRDEDTAVYYCVREYSSSWTGWFDPWGQGTLVTVSS

SEQ ID NO: 113 - protein, 125 aa, synthetic construct
QVQLVESGGGVVQPGRSLRLSCAASGFTFSIYGMHWVRQAPGKGLEWVAVIWHDGSIKYYAESVKGRFTISRDNSKNTLYLQMNSLRVEDTAVYYCAKVGEGEDYSDSSGYFDYWGQGTLVTVSS

SEQ ID NO: 114 - protein, 123 aa, synthetic construct
QVQLVESGGGVVQPGRSLRLSCAASGFPFSTYGMHWVRQTPGKGLEWVAIIWHDGSKKYYADSVEGRFTISRDNSKKTLYLQMNSLRTEDTAVYYCARPGERRLQKSGDFDKWGQGTLVTVSS

SEQ ID NO: 115 - protein, 123 aa, synthetic construct
QVQLVESGGGVVQPGRSLRLSCAASGFTFNSYGMHWVRQAPGKGLEWVAVIWYDGSNKYYADSVKGRFTISRDNSKNTLYLQMNSLRAEDTAVYYCAKDGRDSSDNGGIFDYWGQGTLVTVSS

SEQ ID NO: 116 - protein, 124 aa, synthetic construct
QVQLVESGGGVVQPGRSLRLSCAASGFTFSSYGMHWVRQAPGKGLEWVAVIWYDGSNKFYADSVKGRFTISRDNSKNTLYLQMNSLRVDDTAVYYCAKALGGEEGDSCCPFDIWGQGTMVTVSS

SEQ ID NO: 117 - protein, 124 aa, synthetic construct
QVQLVESGGGVVQPGRSLRLSCAASGFTFSNYGMHWVRQAPGKGLEWVAVIWYDGSKKYYADSLKGRFTISRDNSKNTLYLQMTSLRAEDTAVYYCARDGRGDYGGNSAHFDYWGQGTLVTVSS

SEQ ID NO: 118 - protein, 124 aa, synthetic construct
QEQLVESGGGVVQPGRSLRLSCAASGFTFSSYGMHWVRQTPGRGLEWVAVIWYDGSKKYYGDSVRGRFTISRDNSKNTLYLQMNSLRAEDTAVYYCARDLRGGYYLRDGGFDYWGQGTLVTVSS

SEQ ID NO: 119 - protein, 120 aa, synthetic construct
EVQLVESGGVLVKPGGSLRLSCAASGFTLTDAWMSWVRQAPGKGLEWVGRIKSKTHGGTTDYAAPVKARFTISRDESKSTLYLQMNSLKTEDTAVYYCTTAEARRDLTSWGQGTLVTVSS

SEQ ID NO: 120 - protein, 123 aa, synthetic construct
QVQLVESGGGVVQPGRSLRLSCTASGFTFSNYGMHWVRQAPGKGLEWVAVIWYDGSNKYYADSVKGRFTISRDNSKNTLYLQMNSLRAEDTAVYYCARDGPATVTTWNYFDYWGQGTLVTVSS

SEQ ID NO: 121 - protein, 125 aa, synthetic construct
QVQLVESGGGVVQPGRSLRLSCAASGFTFSNYGMHWVRQAPGKGLEWVSLISYDGGRTFYADSVKGRFTISRDNSRNTLYLQMNSLRTEDTAMYYCARVGYGSYYTATTNPVDHWGQGTLVTVSS

SEQ ID NO: 122 - protein, 117 aa, synthetic construct
QVQLVQSGAEVKKPGASVKVSCKASGYTGYYIHWVRQAPGQGLEWMGWINPNDGGTIYALKFQGRVTMTRDTSISTTYMELSRLSSDDTAIYYCATLTVAAAATDYWGQGTLVTVSS

SEQ ID NO: 123 - protein, 125 aa, synthetic construct
QVQLVQSGAEVKKPGASVKVSCKASGYTFTSYDINWVRQATGQGLEWMGWMNPNSGNTVYAQKFQGRVTMTRNTSISTAYMELSSLRSEDTAVYYCARGGYCSSSSCYEVEFDPWGQGTLVTVSS

SEQ ID NO: 124 - protein, 122 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAKVGEYGDYGDGMDVWGQGTTVTVSS

SEQ ID NO: 125 - protein, 126 aa, synthetic construct
QVQLVESGGGVVQPGRSLRLSCASSGFTFSGYGMHWVRQTPDKGLEWVAIIWYDGSKKYYADSVKGRFTISRDNSRNTLYLQMNSLRAEDTAVYYCARVLNDHVDYGSKSGAFDIWGQGTMVTVSS

SEQ ID NO: 126 - protein, 122 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDRSHLDRTRAFDPWGQGTLVTVSS

SEQ ID NO: 127 - protein, 125 aa, synthetic construct
QVQLVESGGGVVQPGRSLRLSCAASGFTFSSYGMHWVRQAPGKGLEWVAIIWYDGSNKYYADSVKGRFTISRDNSKNTLYLQMNSLRAEDTAVYYCAKDGVDYYDSSGYSQLDNWGQGTLVTVSS

SEQ ID NO: 128 - protein, 123 aa, synthetic construct
QEQLVESGGGVVQPGRSLRVSCAASGFTFSNHGMHWVRQAPGKGPEWVAVIWHDGSKKFYADSIKGRFTISRDNSKNTLYLQMNSLTAEDTAVYYCARVMASSRSSELPFDIWGLGTMVTVSS

SEQ ID NO: 129 - protein, 128 aa, synthetic construct
QVQLVESGGGVVQPGRSLRLSCAASGFTFSSYGMHWVRQTPGKGLEWVAVVWYDGSNKYYAESVKGRFTISRDNSKNTLYLQMNSLRAEDTAVYYCARVLDYGDYGQSEDRFGAFDIWGQGTLVTVSS

SEQ ID NO: 130 - protein, 121 aa, synthetic construct
QVQLVESGGGVVQPGRSLRLSCAASGFTFRNYGMHWVRQAPGKGLEWVAVIWYDGSREFYADSVEGRFTTSRDNSKNTLYLQMNSLRAEDTAVYYCARDRGGSHDREFDYWGQGTLVTVSS

SEQ ID NO: 131 - protein, 122 aa, synthetic construct
EVQLVESGGGLVQPGRSLRLSCTASGFTFGDYAMSWVRQAPGKGLEWVGFIRSKAHGGTTEYAASVKGRFTISRDDSKSIAYLQMNSLKTEDTAVYYCSRVSLVPAAPLDYWGQGTLVTVSS

SEQ ID NO: 132 - protein, 121 aa, synthetic construct
EVQLVESGGGLVQPGRSLRLSCTASGFTFADYAMSWVRQAPGKGLEWVGFIRSKAFGGSTEYAASVKGRFTISRDDSKSIAYLQMNGLKTEDTAVYSCTRIGIAARACDYWGQGTLVTVSS

SEQ ID NO: 133 - protein, 123 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARVADEVLEWSGAFDYWGQGTLVTVSS

SEQ ID NO: 134 - protein, 118 aa, synthetic construct
EVQLLESGGGLVQPGGSLRLSCAASGFTFSSYAMSWVRQAPGKGLEWVSVIYNDGTSTYYADSVKGRFTISRDNSKNTLYLQMNSLRAEDTAVYYCAKEDYGNNSNYWGQGTLVTVSS

SEQ ID NO: 135 - protein, 118 aa, synthetic construct
QVQLVESGGGVVQPGRSLRLSCAASGFTFSSYGMYWVRQAPGKGLEWVAVIWYDGSNKHYADSVKGRFTISRDNSKNTVYLQMNSLRAEDTAVYYCAKGKGEGWFDPWGQGTLVTVSS

SEQ ID NO: 136 - protein, 122 aa, synthetic construct
QVQLVESGGGVVQPGRSLRLSCAASGFTFSSYGMHWVRQAPGKGLEWVAVIWYDGSYKYYADSVKGRFTVSRDNSKNTLYLQMNSLRAEDTAVYYCARAYYDSSGPTAFDYWGQGTLVTVSS

SEQ ID NO: 137 - protein, 122 aa, synthetic construct
EVQLVQSGAEVKKPGESLKISCKGSGYSFTSCWIGWVRQMPGKGLEWMGIIYPGDSDTRYSPSFQGQVTISADKSISTAYLQWSSLRASDTAMYYCARHYCDSSGYYYKDYWGQGTLVTVSS

SEQ ID NO: 138 - protein, 123 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAKVRYSSSWYGDAFDIWGQGTMVTVSS

SEQ ID NO: 139 - protein, 124 aa, synthetic construct
QVQLVESGGGVVQPGRSLRLSCAASGFTFSSYGMHWVRQAPGKGLEWVAIIWYDGSYKYYADSVKGRFTISRDNSKNTLYLQMDSLRAEDTAVYYCARALGNDYGDYGGFDYWGQGTLVTVSST

SEQ ID NO: 140 - protein, 123 aa, synthetic construct
QVQLVESGGGVVQPGRSLRLSCAASGFTFSNYGMHWVRQAPGKGLEWVALIWYDGSNEFYADSVEGRFTISRDNSQNTLYLQMNSLRAEDTAVYYCARSGAYHHGSSGYVDYWGRGTLVTVSS

SEQ ID NO: 141 - protein, 121 aa, synthetic construct
QVRLVQSGAEVKKPGASVKVSCKASGYSFSDYYLHWVRQAPGQGLEWMGWINPNSGDTNYAQKFQGRVTMTRDTSIITAYMELSRLRSNDTAVYYCARSRTWGWHAEGDFWGQGTRVTVSS

SEQ ID NO: 142 - protein, 128 aa, synthetic construct
QVQLVESGGGVVQPGRSLRLSCAASGFTFSTYGMHWVRQAPGKGLEWVAIISYDGSNKYYADSVKGRFTFSRDNSKNTLYLQMNSLRAEDTAVYYCAKDGVGEYCSGATCSSGAFDIWGQGTMVTVSS

SEQ ID NO: 143 - protein, 121 aa, synthetic construct
QVQLVESGGGVVQPGRSLRLSCAASGFTFSSYGMHWVRQAPGKGLEWVAVIWYDGSNKFYADSVEGRFTISRDNSKNTLYLQMNSLRPEDTAVYYCAKVYFRGGPGAFDIWGQGTMVTVSS

SEQ ID NO: 144 - protein, 119 aa, synthetic construct
QVHLVESGGGVVQPGRSLRLSCAASGFTFSNYAMHWVRQAPGKGLEWVAVISYHERNKYYADSVKGRFTISRDNSKNTLFLQMDSLTAADTSVYYCARDGHSSSYFDYWGQGTLVTVSS

SEQ ID NO: 145 - protein, 122 aa, synthetic construct
QAQSVESGGGVVQPGRSLRLSCAVSGFTFRNFGMHWVRQAPGKGLEWVAVIWFDGSQTFYADSVEGRFTVSRDNSKSTLYLQMNSLRVDDTAVYYCARDRGYSGSWGYFDNWGQGTLVTVSS

SEQ ID NO: 146 - protein, 122 aa, synthetic construct
QGQVVESGGGVVQPGRSLRLSCAASGFTFSNFGMHWVRQAPGKGLEWVAVIWHDGSNTFYADSVEGRFTISRDNSKNTVSLQMNSLRADDTAMYYCARDRGYSGSWGYFDHWVQVTMVIVSS

SEQ ID NO: 147 - protein, 122 aa, synthetic construct
QVQPVQSGGGVVQPGRSLRLSCAVSGFTFSNFGMHWVRQAPGKGLEWVGVIWHDGSKKFYADSVEGRFTISRDNSKNTLYLQMNSLRAEDTAVYYCARDRGYGTSWGYFDFWGQGTLVTVSS

SEQ ID NO: 148 - protein, 122 aa, synthetic construct
QVQLVESGGGVVQPGGSLRLSCVASGFIFSNYGMYWVRQAPGKGLEWVGVIWFDGSNIFYGDSVEGRFTISRENSKNTLYLQMNSLRAEDTAVYYCARDRGYTGSWGYFGYWGQGTPVTVSS

SEQ ID NO: 149 - protein, 122 aa, synthetic construct
QVQLVESGGGVVQPGRSLRLACEASRFIFRNFGMHWVRQAPGKGLEWVGVIWHDGSNEFYADSVEGRFTISRDNSKNTLYLQMNNLRVDDTAVYYCARDRGYGGSWGYFDYWGRGTLVTVSS

SEQ ID NO: 150 - protein, 122 aa, synthetic construct
QVQLVEPGGGVVQPGRSLRLSCEVSTFTFSNFGMHWVRQAPDRGLEWVAVIWFDGSKKYYADSVEGRFSISRDNSKNTLYLQMNSLRPEDTAVYYCARDRGYSGSWGYFDHWGQGTLVTVSS

SEQ ID NO: 151 - protein, 123 aa, synthetic construct
QVQLVESGGGVVQPGRSLRLSCAASGFPFNTYGMHWVRQTPGKGLEWVAIIWYDGSQKYYADSVQGRFTISRDNHKDTLSLQMNGLRVEDTAVYFCVRVRFSVGPHGSAFDLWGQGTMVTVSS

SEQ ID NO: 152 - protein, 123 aa, synthetic construct
QVHLVESGGGVVQPGRSLRLSCAASGFTFSTYGMHWVRQAPGKGLEWVAIIWYDGSQKYYAGSVEGRFTISRDNSKNTLYLQMNSLRAEDTAVYHCARVRYSSSHHGSAFDLWGQGTMVTVSS

SEQ ID NO: 153 - protein, 123 aa, synthetic construct
QVQLVESGGGVVQPGRSLRLSCAASGFMFNTYGMHWVRQTPGKGLEWVAIIWYDGSYKFYADSVEGRFTISRDNSKNTLYLQMNSLRAEDTAVYYCARVRHSRSQHGSAFDLWGQGTTVTVSS

SEQ ID NO: 154 - protein, 121 aa, synthetic construct
QAQLVESGGGVVQPGRSLRLSCAASGFSFSTYGMHWVRQAPGKGLEWVAVIWYDGSIKHHAKSVEGRFTISRDNSKNTLYLQMNSLRAEDTAVYYCTRDLDYSNHNYFDPWGQGTLVTVSS

SEQ ID NO: 155 - protein, 121 aa, synthetic construct
QEQLVESGGGVVQPGRSLRLSCVASGFSFGSYGMHWVRQAPGKGLEWVSVIWFDGSNTNYADSVRGRFTISRDNSRNTVYLKMNSLRVEDTAVYYCARDFDFYAHNYFDPWGQGTLVTVSS

SEQ ID NO: 156 - protein, 121 aa, synthetic construct
QEQLVESGGGVVQPGRSLRLSCEASGFSFRSYGMHWVRQAPGKGLEWVAVIWFDGSNTLYADSVRGRFTVSRDNAKNTLYLQMNNLRAEDTAVYYCAKDLDYFNHNYLDPWGQGTLVTVSS

SEQ ID NO: 157 - protein, 121 aa, synthetic construct
QVQLVESGGGVVQPGRSLRLSCAASGFDFNNYGMHWVRQAPGKGLEWVAVIWFDGSQKFHAASVEGRFTISRDNSKNTLYLQMNSLRAEDTAVYYCARDLDYYHHNYFGHWGQGTLVTVSS

SEQ ID NO: 158 - protein, 121 aa, synthetic construct
QEQLVESGGGVVQPGRSLRLSCAASGFTFSSYGMHWVRQAPGKGLEWVAVIWHDGSNAYYADSVRGRFTISRDNSKNTLYLQMNSLRAEDTAVYYCARDFDYFNHNYFDPWGQGTLVTVSS

SEQ ID NO: 159 - protein, 121 aa, synthetic construct
QVQLVQSGGGVVQPGRSLRLSCAASGFIFSNYGMHWVRQAPGKGLEWVAVIWYDGSTEHYAASVEGRFSVSRDNSKNTLYLQMNSLRVEDTAVYYCARDFDYSNHNYFGPWGQGALVTVSS

SEQ ID NO: 160 - protein, 121 aa, synthetic construct
QVQLVESGGGVVQPGRSLRLSCEVSGFDFNNYGMHWVRQTPGKGLEWLAVIWYDGSNKFYADSVKGRFTISRDNSKNTLFLQMNSLRADDTAVYYCARDLDYYNHNYFHPWGQGTLVTVSS

SEQ ID NO: 161 - protein, 121 aa, synthetic construct
QELLVESGGGVVQPGRSLRLSCAASGFGFSGYGMHWVRQAPGRGLEWVAVIWYDGSKKYYGDSVKGRFTISRDNSNDTLYLQMNSLRVEDTAVYYCARDFDYFSPNYFDPWGQGTLVTVSS

SEQ ID NO: 162 - protein, 121 aa, synthetic construct
QEQLVESGGGVVQPGRSLTLSCVASGFSFRSYGMHWVRQAPGKGLEWLAVIWFDGSNKYYADSVKGRFTISRDNSNNTLYLQMNSLRADDTAVYYCARDADYYKHNYFDPWGQGVLVTVSS

SEQ ID NO: 163 - protein, 121 aa, synthetic construct
QVQLVESGGGVVQPGRSLRLSCAASGFSFSSYGMHWVRQAPGKGLEWVAIIWYDGSNTYYADSVKGRFTISRDNSKNTLYLQMNSLRAEDTAVYYCAGDHDYFNHNYFDPWGQGTLVTVSS

SEQ ID NO: 164 - protein, 121 aa, synthetic construct
QVQLVESGGGVVQPGRSLRLSCAASGFSFSNYGMHWVRQAPGKGLEWMAVIWHDGSNKFYADSLKGRFTISRDNSNNTLYLQMNSLRAEDTAVYYCTRDLDYYNHNYFDPWGQGTLVTVSS

SEQ ID NO: 165 - protein, 124 aa, synthetic construct
QVQLVESGGGVVQPGRSLRLSCAASGFTFSSQGMHWVRQAPGKGLEWVAVIWYDGSNKYYADSVKGRFTISRDNSKNTLYLQMNSLRAEDTAVYYCARDGTNWDESSGYILDHWGQGTLVTVSS

SEQ ID NO: 166 - protein, 122 aa, synthetic construct
EVQLVESGGGLVQPGRSLRLSCTASGFTFGDYAMSWFRQAPGKGLEWVGFIRITAYGGTPERAASVKGRFIFSRDDSKSIAYLQMSTLKTEDTAVYYCARVRSSDFRDMDVWGKGTTVTVSS

SEQ ID NO: 167 - protein, 122 aa, synthetic construct
EVQLVESGGGLVQPGRSLRLSCTASGFTFGDYAMSWFRQAPGKGLEWVGFIRSKAYGGTTEYAASVKGRFTISRDDSKSIAYLQMNSLKTEDTAVYYCTRVRSYDFRDMDVWGKGTTVTVSS

SEQ ID NO: 168 - protein, 120 aa, synthetic construct
QVQLVESGGGVVQPGRSLRLSCTASGFIFTNYGMHWVRQAPGKGLEWVAVIWYDGSNEFYADSVEGRFTISRDNSKNTLYLQMNSLRAEDTAVYYCARSYLSSWLGFDYWGQGTLVTVSS

SEQ ID NO: 169 - protein, 122 aa, synthetic construct
QVQLVESGGGVVQPGRSLRLSCAASGFTFSNSGMHWVRQAPGKGLEWVALISYDGSNKFYADSVRGRFTISRDNSKNTLFLQMNSLRAEDTAVYYCATDSLYYYDAHLFDYWGQGTLVTVSP

SEQ ID NO: 170 - protein, 122 aa, synthetic construct
QVQLVESGGGVVQPGRSLRLSCAASGFIFSSHGMHWVRQAPGTGLEWVALTSYDGSNKFYADSVKGRFTISRDNSKNTVYLQMNSLRAEDTAVYYCATDSLDFYNYHLFDYWGQGTLVTVSS

SEQ ID NO: 171 - protein, 122 aa, synthetic construct
EVQLVESGGGLVQPGGSLRLSCAASGFTFSSYEMNWIRQAPGKGLEWVSYISSSGSAKYYADSVKGRFSISRDNAENSLSLQMNSLRAEDTAVYYCARDHSTSAREYGLDVWGQGTTVTVSS

SEQ ID NO: 172 - protein, 122 aa, synthetic construct
EVQLVESGGGLVQPGGSLRLSCVVSGFTFSNYVMNWVRQAPGKGMEWVSFISSSGSKTRYADSVKGRFTISRDNARNALYLQMNSLRDEDTAVYYCARDHPSSHREYGLDVWGQGTTVIVSS

SEQ ID NO: 173 - protein, 122 aa, synthetic construct
EVQLVESGGGLVQPGGSLRLSCAASGFTFSSYEMNWVRQAPGKGLEWVAYSSRSGTTMLYADSVKGRFTSSRDNAKNSLYLQMSSLRAEDTAVYYCARDHSTSASEYGLDVWGQGTTVTVSS

SEQ ID NO: 174 - protein, 121 aa, synthetic construct
QVHLVESGGGVVQPGRSLRLSCAASGFTFSNSAMHWVRQAPGKGLEWVALIWYDGSNKFYAHSVEGRFTISRDNSKNTLYLQMNSLRAEDTAVYYCARAGPYDSSGYLDYWGQGTLVTVSS

SEQ ID NO: 175 - protein, 121 aa, synthetic construct
QVQLVESGGGMVQPGRSLRLSCLVSGITFSNYGMHWVRQAPGKGLEWVALIWYDGSNKFYADSVEGRFTISRDNSKNTLYLHMSSLRAEDTAVYYCARAGYYDNSGYLDHWGQGTLVTVSS

SEQ ID NO: 176 - protein, 121 aa, synthetic construct
QVQLVESGGGVVQPGRSLRLSCAASRFPFSNYGMHWVRQAPGKRLEWVALIWSDGSNEFYHHSVEGRFTISRDNSKNTLFLQMNSLRAEDTAVYFCARAGRYDNGGYLDSWGQGTLVTVSS

SEQ ID NO: 177 - protein, 121 aa, synthetic construct
QVQLVESGGGVVQPGRSLRLSCAASGFSFRNSGMHWVRQAPGKGLEWVALIWHDGSNQFYADSVEGRFSISRDNSKNTLYLQMNSLRAEDTAVYYCARAGYYDSSGYFDSWGQGTLVTVSS

SEQ ID NO: 178 - protein, 121 aa, synthetic construct
QVQLVESGGGVVQPGRSLRLSCEASGFIFSNSGMHWVRQVPGKGLEWVALIWYDGSNKFYVKSVEGRFTISRDNSKNTLYLQMNSLRAEDTAIYFCARAGRSDNGGYLDYWGQGALVAVSS

SEQ ID NO: 179 - protein, 121 aa, synthetic construct
QVHLVESGGGVVQPGRSLRLSCAASGFIFSNFGMHWVRQAPGKGLEWVALIWYDGSNKFYAESVDGRFSISRDNSKNTLYLQLNNLRAEDTAVYFCARAGYDDDSGYLDSWGQGTLVTVSS

SEQ ID NO: 180 - protein, 121 aa, synthetic construct
QVQLVESGGGVVQPGGSLRLSCTASRFTFRNFGMHWVRQAPGKGLEWLALIWYDGSNEFYADSVEGRFTISRDNSKNTLYLQMNGLRAEDTAVYYCARAGQNDDSGYLDYWGHGSLVTVSS

SEQ ID NO: 181 - protein, 121 aa, synthetic construct
QVQLVESGGGVVQPGTSVRLSCDASGFTFRNYGMHWVRQVPGKGLEWVALTWYDGSNEFYAESVEGRFTISRDNSKNTLFLQMNSLRAEDTAVYYCARAGYYDNSGYLDYWGQGTLVTVSS

SEQ ID NO: 182 - protein, 113 aa, synthetic construct
QVQLVESGGGLVQPGGSLRLSCAASGFTFSNYWMTWVRQAPGKGLEWVANINEDGSEKDYVDSVKGRFTISRDNPKNSLYLHMDSVRVEDTAVYYCARYLNSWGQGILVTVSS

SEQ ID NO: 183 - protein, 114 aa, synthetic construct
ELKLVESGGGLVQPGGSLRLSCEASGFKFSNYWMSWVRQAPGKGLEWVANINQEGSQMDYLDSVKGRFTISRDNAKNLLLLQMNNLRAEDTAVYYCARYLNFWGPGILVTVSST

SEQ ID NO: 184 - protein, 121 aa, synthetic construct
EVQMVESGGGLVKPGGSLRLSCAASGFTFSNAWMSWVRQAPGKGPEWVGRIRSKTDGGTTDYAAPVKGRFTISRDDSKNTLYLQMNSLKTEDTGVYYCITDRDFYRSGGDWGQGTLVTVSS

SEQ ID NO: 185 - protein, 121 aa, synthetic construct
EVHLVESGGGLVKPGGSLRLSCAVSGFTFSNAWMSWVRQAPGKGLEWVGRIRSKTDGGTIDYAAPVIGRFTISRDDSKSTLYLQMNSLKIEDTAVYYCITDRDFYRSGGHWGQGSLVTVSS

SEQ ID NO: 186 - protein, 121 aa, synthetic construct
EVQLVESGGGLVEPGGSLRLSCAASGFTFNNAWMSWVRQAPGKGPEWVGRITSKTDGGTTDYAAPVKGRFTISRDDSKDTLYLQMNRLKTEDTAVYYCITDRDFYRSGGSWGQGTPVTVSS

SEQ ID NO: 187 - protein, 121 aa, synthetic construct
EVQLEESGGGLVKPGGSLRLSCAASGFTFSKAWMSWVRQAPGKGPEWVGRIESKGDGGTIEYAAPVKGRFTISRDDSKNMLYLQMNSLKTEDTAVYYCITDRDFYRSGGDWGQGTLVTVSS

SEQ ID NO: 188 - protein, 121 aa, synthetic construct
DLQLVESGGGLVKPGESLRLSCVGSGFPFNNAWMNWVRQAPGKGPEWVGRITSKNDGGKTDYAAPVKGRFTISRDDSKSALFLHMNRLRTEDTAVYYCITDRDFYRSGGSWGQGTPVTVSS

SEQ ID NO: 189 - protein, 121 aa, synthetic construct
EVQLVESGGGLVKPGGSLRLSCATSGFTFSNAWMSWVRQAPGKGLEWVGRMKSKIYGGTRDYGAPVKGRFTISRDDSKDTLYLQMNSLKTEDTAVYYCITDRDLYSSGGYWGQGTLVTVSS

SEQ ID NO: 190 - protein, 121 aa, synthetic construct
EVQLVESGGGLVKPGGSLRLSCAVSGFIFSDAWMSWVRQAPGKGPEWVGRITSKSDGGTRDYAAPVRGRFTISRDDSRNTLYLQMNSLKTEDTAIYYCITDRDFYRSGGNWGQGTLVTVTS

SEQ ID NO: 191 - protein, 121 aa, synthetic construct
EVQLVESGGGLVKPGGSLRLSCVASGFSFTNAWMSWVRQAPGRGPEWVGRIESKTDGGTIEYAAPVKGRFTISRDDSKNTLYLQLNSLKTEDTGVYYCITDRDFYRSGGSWGQGTLVTVSS

SEQ ID NO: 192 - protein, 121 aa, synthetic construct
DVQLVESGGGLVKPGGSLRVSCAASGFTFGNAWMSWVRQAPGKGPEWVGRVRSKSDGGTRDYAAPVKGRFTISRDDSKNTLYLQMNNLKTEDTAVYYCITDRDFYRSGGYWGQGTLVTVSS

SEQ ID NO: 193 - protein, 124 aa, synthetic construct
QVQLVESGGGVVQPGTSLRLSCEVSGFTFSSYGMHWVRQAPGKGLAWVAVIWYDGSTQFYADSVEGRFTISRDNSKNTLYLQMTSLRVEDTAVYYCARVGMGAQDDSSGYYDYWGQGTLVTVAS

SEQ ID NO: 194 - protein, 124 aa, synthetic construct
QLQLVESGGGVVQPGRSLRLSCVVSGFSFSSYGMHWVRQAPGKGPEWVAVIWHDGSKYYHAPSVEGRFTISRDNSKNTLYLQMNSLRVDDTAVYYCARVGMGDYKDSSGYYDQWGQGTMVIVSS

SEQ ID NO: 195 - protein, 124 aa, synthetic construct
QVQLVESGGGVVQPGRSLRLSCEASGFTFSSYGMHWVRQTPDKGLEWVAVIWHDGSKKYYPDSVEGRFTVSRDNSRNTLYLQMNSLRAEDTAVYYCARVGMGAQSDSYGYYDYWGQGTLVTVSS

SEQ ID NO: 196 - protein, 124 aa, synthetic construct
QVQLVESGGGVVQPGRSLRLSCAASGFIFRNYGMHWVRQTPGRGLEWVAVIWYDGSNKYYADSVEGRFTISRDNSKNTLYLQMNSLTAEDAAIYYCARVGMGAQEDKSGYYDLWGQGTLVTVAS

SEQ ID NO: 197 - protein, 124 aa, synthetic construct
LVQLVESGGGVVQPGRSLRLSCETSGFTFSNFGMHWVRQTPGKGLEWVAVIWHDGSQKYYGDSVEGRFTISRDNSKKTLYLQLNSLRADDTAVYYCARDIRGAFKVTDGGFDYWGQGTLVTVSS

SEQ ID NO: 198 - protein, 124 aa, synthetic construct
QVQLVESGGGMVQPGRSLRLSCAASGFTFSNFGMHWVRQAPGKGLEWVAVIWFDGSQKYYAHFVKGRFTISRDNSNDMLYLQMSSLGAEDTAIYYCARDIRGAYRVTDGGFDYWGQGTLVTVSS

SEQ ID NO: 199 - protein, 124 aa, synthetic construct
QVQLVESGGGAVQPGRSLRLSCEASGFVFDNFGMHWVRQAPGKGLEWVAVIWHDGSQKYYGDSVKGRFTVSRDNAKNTLYLQMNSLRAEDTAVYYCATDIRGAYKVTDGGFDSWGQGTLVTVSS

SEQ ID NO: 200 - protein, 124 aa, synthetic construct
QVQLVESGGGAVEPGRSLRLSCEASGFVFDNFGMHWVRQAPGRGLEWVAVIWHDGSQKYYGDSVKGRFTVSRDNFKNTLYLQMNSLRAEDTGLYYCATDIRGAYKVTDGGFDLWGQGTLVTVSS

SEQ ID NO: 201 - protein, 121 aa, synthetic construct
QVQLVESGGGVVPPGRSLRLSCATSGFTFSNYGMHWVRQAPGKGLEWVAIIWYDGSKNFYAASVEGRFTISRDNSKNTLYLQMNSLRVEDTAVYYCARAAYYDTSGYGDYWGQGTLVTVSS

SEQ ID NO: 202 - protein, 121 aa, synthetic construct
QVQLVESGGGVVQPGGSLRLSCEASGFTFRNYGMHWVRQAPGKGLEWVAVIWHDGSKENYADSVEGRFTISRDNSEDALYLEMSSLRADDTGVYYCARAGYYDTSGYGAHWGQGTLVTVSS

SEQ ID NO: 203 - protein, 121 aa, synthetic construct
QVQLVESGGGVVQPGRSLRLSCAASMFTFRNYGMHWVRQAPGKGLEWVAVIWHDGSREFYADSVEGRFTISRDNSKNTLYLEMNSLRAEDTAIYYCARAAYYENSGYGDYWGQGTLVTVSS

SEQ ID NO: 204 - protein, 124 aa, synthetic construct
QVQLVESGGGVVQPGRSVRLSCAASGFMFSTYGMHWVRQAPGRGLEWVALIWYDGSNKYYRDSVKGRFTISRDNSKEILYLQMNSLRAEDTAVYYCARDGADYFDPSGGAFDIWGLGTMVTVSS

SEQ ID NO: 205 - protein, 125 aa, synthetic construct
QVQLVESGGGVVQPGRSLRLSCAASGFTFRNYGMHWVRQAPGKGLEWVALIWYDASNEYYLESVKGRFTISRDNTKNTLYLQMNDLRAEDTAVYYCARVGYHTNYYDSSGFLDYWGQGTLVTVSS

SEQ ID NO: 206 - protein, 125 aa, synthetic construct
QVQLVESGGGVVQPGRSLRLSCAASGFTFRSYGMHWVRQAPGKGLEWVAVIWYDGSNKYYADSVKGRFTISRDNSKNTLYLQMNSLRAEDTAVYYCARVGDGTTYHDSSGYLDHWGQGTLVTVSS

SEQ ID NO: 207 - protein, 125 aa, synthetic construct
QVQLVESGGGVVQPGRSLSLSCAASGFTFSNYGMHWVRQAPGKRLEWVAVIWHDGSNKYYAVSVKGRFTISRDNSKNTLYLQMNNLRAEDTAVYYCARVGDGTTNSDSSGYLDYWGQGTLVTVSS

SEQ ID NO: 208 - protein, 126 aa, synthetic construct
QVQLVESGGGVVQPGRSLRLSCAASGFTFNNYGMHWVRQAPGKGLEWVAVIWYDGSKKYYVDSVKGRFTISRDNSKNTLYLQMNSLRAEDTAVYYCVRDGRYRTSHQTSTGFFDLWGQGTLVTVSS

SEQ ID NO: 209 - protein, 127 aa, synthetic construct
QVQLQESGPGLVRSSETLSLTCTVSGDSMRRYFRNWIRQSPGRGLEWIGAIFDNGSTRYNPSLKSRVTMSVDVSKNQFSLRLSSVTAADTAVYYCARARDYYDFLTRTYRNFYFDYWGQGTLVTVSS

SEQ ID NO: 210 - protein, 123 aa, synthetic construct
EVQLLESGGGLVQPGGSLRLSCAASGFTFSNHAMSWVRQAPGKGLQWVSAISGSGDRTYYADSVKGRFTISRDNSKNTLYLQMNSLRADDTAVYYCAKDQGGYSIYPLGMDVWGQGTTVTVSS

SEQ ID NO: 211 - protein, 122 aa, synthetic construct
EVQLVESGGGLVQPGGSLRLSCAASGFTFRHYAMNWVRQAPGKGLEWVSSISGSGGNTYYAVSVKGRFTISRDNSENTLYLQMNSLRAADTAVYYCARSLDYGGNSLSPEYWGQGTLVIVSS

SEQ ID NO: 212 - protein, 122 aa, synthetic construct
EVHLVESGGGLVKPGGSLRLSCAASGFTFSSYTMTWVRQAPGKGLEWVSSISSTSTHIYYADSVRGRFTISRDNAKNSLYLQINSLGAEDTGLYYCARIFGIASADTTFDQWGQGTLVSVSS

SEQ ID NO: 213 - protein, 124 aa, synthetic construct
QVQLVESGGGLVKAGGSLRLSCAASGFAFKDYYMSWIRQAPGKGLEWISYISGRGSTIYYADSAKGRFTISRDNAKNSLYLQMSSLRAEDSAIYYCAGGGGYPQLTHSYYMDVWGKGTTVTVSS

SEQ ID NO: 214 - protein, 126 aa, synthetic construct
AVQLVESGGGLVKPGGSLRLSCAASGFTFSLYSMTWVRQAPGKGLEWVSSISTSSSYIYYADSVKGRFTISRDNAKKTLYLHMNSLRAEDTAVYYCARDRGTALRNFEWSDASDIWGQGTKVTVSS

SEQ ID NO: 215 - protein, 118 aa, synthetic construct
QVQLVESGGAVVQPGKSLRLSCAASGFTFSNYAVHWVRQAPGKGLEWVAFISYDSRNKYYADSVKGRFTISRDNSKNTLYLHMDSLRPEDTAVYYCARDLLVGVSQYWGQGTLVTVSS

SEQ ID NO: 216 - protein, 123 aa, synthetic construct
QMQLVQSGPEMKKPGSSVKVSCKAPRGTFSSFTIGWVRQAPGHGLEWVGMIVPLFGIPKYAPKLQGRVTISADDSTDTAYMELSSLRSEDTAIYYCARDRYDWNERVWYFDLWGRGTLVTVSS

SEQ ID NO: 217 - protein, 125 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARVGYGGHYYDSSGYYDYWGQGTLVTVSS

SEQ ID NO: 218 - protein, 125 aa, synthetic construct
QEQLVESGGGVVQPGRSLRLSCVASGFIFRNFGMHWVRQAPGKGLEWVAVIWYDGSYEFYADSVKGRFTISRDNSKNTLYLQMNSLRANDTAVYYCASDRRNPGIGAAVGAFDIWGQGTMVTVSS

SEQ ID NO: 219 - protein, 123 aa, synthetic construct
EVQLVESGGGLVKPGGSLRLSCAASGFTFSSYTMNWVRQAPGKGLEWVSCITSSSSYIYYADSVKGRFTISRDNAKNSLYLQMDSLRAEDTAVYYCARVVGFCVGGSCYSDYWGQGTLVTVSS

SEQ ID NO: 220 - protein, 133 aa, synthetic construct
QVQLVESGGGVVQPGRSLRLSCAASGFTFSSYAMHWVRQAPGKGLEWVAVISYDGSNKYYADSVKGRLTISRDNSKNTLYLQMNSLRAEDTAVYYCARQGGYCTNGVCSEGANYYNYYGMDVWGQGTTVTVSS

SEQ ID NO: 221 - protein, 124 aa, synthetic construct
QVQLVESGGGVVQPGRSLRLSCAASGFTFSNYGMHWVRQAPGKGLEWVAVIWYDGSNKYYADSVKGRFTISRDNSKNTLYLQMNSLRAEDTAVYYCARPQGLYGSTNYVTFDIWGQGTMVTVSS

SEQ ID NO: 222 - protein, 121 aa, synthetic construct
QVQLQESGPGLVKPSGTLSLTCAVSGGSISSSNWWNWVRQPPGKGLEWIGEIYHSGSPNYNPSLKSQVTISVDKSNNQLSLKLTSVTAADTAVYYCASKHYSLFLGWVVSWGQGTLVTVSS

SEQ ID NO: 223 - protein, 123 aa, synthetic construct
QVQLVESGGGVVQPGRSLRLSCAASGFVFSNFGMHWVRQAPGKGLEWVAVIWHDGSKKYYTESVEGRFTISRDNSKRTLYLQMNSLTAEDTAVYYCVRDGRTVGATTGWFDPWGQGTLVTVSS

SEQ ID NO: 224 - protein, 120 aa, synthetic construct
QVHLVESGGGVVQPGGSLRLSCAASGFTFRNYGMHWLRQAPGKGLEWVAFIPYDSRDKYYTDSARGRFTISKDNSKNTLYLQMNSLRVEDTAVYYCAKVDIAVAGNLDYWGQGTLVTVSS

SEQ ID NO: 225 - protein, 125 aa, synthetic construct
EVQLLESGGGLVQPGGSLRLSCATSGFIFTNYGMTWVRQAPGKGLEWVSAISGGGDSTYYADSVQGRFTVSRDNSKNTLYLQMNSLRAEDTAVYYCAKEGGFCSSATCYFYMDIWGKGTTVTISS

SEQ ID NO: 226 - protein, 131 aa, synthetic construct
QVQLQESGPGLVKPSQTLSLTCTVSSDSISRGGYFWSWIRQHPGKGLEWIGYIHYSGSTYYNPSLKSRVTISVDTSKNQFSLKLSSVTAADTAVYYCARDRGEYCSGSSCSSYYYYYMDVWGKGTTVTVSS

SEQ ID NO: 227 - protein, 123 aa, synthetic construct
DVELVESGGGLVQPGGSLRLSCAASGFTLSAYWMHWVRQVPGKGLVWVSRINSDGSYTTYVDSVRGRFTISRDNAKNTVYLQMDNLRADDTAIYYCVRPDVDNWNDEGAFQYWGQGTLVTVSS

SEQ ID NO: 228 - protein, 124 aa, synthetic construct
EVQLVESGGGLVKPGGSLRLSCAASGFTFSYYGMNWVRLTPGKGLDWVSSITSSSGYIYYADSVKGRFTISRDNAKNSLYLQMNSLRAEDTAVYYCARDYGSGTYGDYDAFDIWGQGTMVTVSS

SEQ ID NO: 229 - protein, 120 aa, synthetic construct
QVQLQESGPGLVKPSETLSLTCSVSGGSIGTYYWNWIRQSPGKGLEWIAYIFYTGRTTYNPSLESRATISLDMSNSQFSLKLSSMTAADTAVYYCARVDLRRDGYKFDYWGQGTLVTVSS

SEQ ID NO: 230 - protein, 124 aa, synthetic construct
QLQLQESGPGLVKPSETLSLTCAVSGGSIINSRYYWGWIRQPPGKGLEWIGSIFYSGSTYYNASLKRRVTISVDTSKNQFSLKLSSVTAADTAVYYCARLRVGRYSSDWLFDYWGQGTLVTVSS

SEQ ID NO: 231 - protein, 129 aa, synthetic construct
EVQLVESGGGLVQPGGSLRLSCVVSGFTFSSYEMNWVRQAPGKGLEWVSYISSSGSTIYYADSVKGRFTISRDNAKNSLYLQMNSLRAEDTAVYYCAREDSFWSGYVTRREDYYCMDVWGKGTTVTVSP

SEQ ID NO: 232 - protein, 123 aa, synthetic construct
EVQLVESGGGLVQPGGSLKLSCAASGFAFSASAMHWVRQAPGKGLEWVGRVRSKTNNYATAYSASVEGRFTISRDDSKNTAYLQMSSLKTDDTAVYYCARSDMAAPWVAFDIWGQGTMVTVSS

SEQ ID NO: 233 - protein, 124 aa, synthetic construct
EVQLVESGGGLVQPGGSLKLSCAASGFIFSGSGVHWVRQASGRGLEWVGRSRSKVDRYATAYAASVKGRFIISRDDSSNTAYLEMNSLKTEDTAVYYCSTQTGTTGTSANFDLWGQGTLVTVSS

SEQ ID NO: 234 - protein, 119 aa, synthetic construct
QITLKESGPTLVKPTQTLTLTCTFSGFSLNSFGVGVAWIRQPPGRDLEWLALIYWDDDKRYSPSLENRLTITKDTSKNQVVLTMTHMAPADAATYYCAHRTASEQLDYWGQGAPVTVSS

SEQ ID NO: 235 - protein, 126 aa, synthetic construct
EVQLVESGGGLVQPGGPLKLSCVASGFSFSDSAIHWVRQAPGKGLEWLGRMRSKTNNDATVYAASVQGRFTISRDDSRNTAFLQMNSLKTEDTAVYYCTRGEFCGVDCYRDAFDVWGQGTVVTVSS

SEQ ID NO: 236 - protein, 117 aa, synthetic construct
EVQLVESGGGLVQPGGSLRLSCSASGFRFTSYWMSWLRQAPGKGLEWVANIKEDGSVEHYVDSVRGRFTISRDNARNSLYLQMSSLRAEDTAMYYCATASDSCANDWGQGTLVTVSS

SEQ ID NO: 237 - protein, 118 aa, synthetic construct
EVQLVESGGALVQPGGSLKLSCAASGYTFSGSGIHWVRQPFGKGLEWIGHIKNKANNYATDYAASVNGRFTISRDDSKNTAFLQMNNLKTEDSAVYFCARPLSGYDYWGQGTLVTVSS

SEQ ID NO: 238 - protein, 129 aa, synthetic construct
QVQLVESGGGVVQPGRSLRLSCSASGFTLRRHGMHWVRQAPGKGLEWVASISYGEENKYYLDSVKGRFTISRDISKNTLHLQMSSVRTEDTAVYFCAKDNYDSLSGYSTDYNFHGMDVWGQGTTVTVSS

SEQ ID NO: 239 - protein, 130 aa, synthetic construct
QVLLQESGPGLVRPSETLSLTCTVSGGSISRTSHYWGWIRQSPGKGLEWIGSAYYSGSTYYNPSLKSRLTISVDTSKNQFSLRLSSVTAADTAVYYCARHTYDISTGWALFYYYYHIDVWGKGTTVTVSS

SEQ ID NO: 240 - protein, 128 aa, synthetic construct
EVQLVESGGGLIQPGGSLRLSCKPSGFTFSYYSMNWVRQAPGKGLEWVSNIGTTGSPRYYSDSVKGRFTISRDNTNNLLYLQMNSLRAEDTALYYCGRAQPGYSNGWYSAPMVAIDLWGRGTMVTVSS

SEQ ID NO: 241 - protein, 128 aa, synthetic construct
EVQLLESGGGLVQPGGSLRLSCAASGFTFSNYAMSWVRQAPGKGLEWVSIISGSGDSTNYADSVKGRFTISRDNSKKKLYLQMNSLRAEDTAVYYCAKVVPEYTSGWYSYYYGYMDVWGKGTTVSVSS

SEQ ID NO: 242 - protein, 124 aa, synthetic construct
QVHLVQSGAEVKRSGSSVKVSCMASPGTFNSYTISWVRQAPGQGLEWVGRIIPIFGTTNYAPRFKGRVTITADKSTSTAYMELSSLKSEDTAVYYCATMRYYYDRGGPRGFDSWGQGTLVTVSS

SEQ ID NO: 243 - protein, 124 aa, synthetic construct
EVQLVESGGNLVQPGGSLRLSCAGSGFTFSRHWMHWVRQAPGKGLVWVSRVSTDGSSTSYADSVKGRFTISKDNARNTMYLQMNSLRAEDTAVYYCVRGGTFYFGDYNYGMDVWGQGTTVTVSS

SEQ ID NO: 244 - protein, 122 aa, synthetic construct
EVQLVESGGGLVQPGGSLRLSCAASGFTFSSYWMHWVRQAPGKGLVWVSRLNSNGITTSYADSVKGRFTISRDNAKNTLYLQMNSLRDEDTGVYYCVKGGSGWFFDWYFDLWGRGTLVTVSS

SEQ ID NO: 245 - protein, 121 aa, synthetic construct
AEHLLESGGGLGQPGGSLRLSCAASGFSFDDYAMSWVRQAPGKGPEWVSAIFGSGGGTYYADAVKGRFTISRDNSKSTLYLQMNSLRPEDTAVYYCAKEPEGGKGGWLDPWGQGTLVTVSS

SEQ ID NO: 246 - protein, 126 aa, synthetic construct
QVQLVESGGGVVQPGRSLRLSCAASGFTFSNYGMHWVRQAPGKGLEWVAVIWFDGSYKNTADSMRGRFTISRDNSRNTLYLQMNSLRVEDTAVYYCARERSHSSGWYMGGYGMDVWGQGTTVTVLS

SEQ ID NO: 247 - protein, 133 aa, synthetic construct
QVQLQESGPGLVKPSETLSLTCTVSGVSVGTYYWSWVRQPPGKALEWMGYIHHRGSSSYSPSLKSRVTLSVDTSKNQVSLSLHSVTAADTAVYFCARSHPQFSCSSGVCNASSPAEYYGMDVWGQGTTVTVSS

SEQ ID NO: 248 - protein, 128 aa, synthetic construct
EVQILESGGGLAQPGGSLRLSCAASGFTFNYFAMSWVRQAPGKGLEWVSAIHPSGGATYYADSVKGRFTISRDNSRNTLYLQMNSLRAEDTALYYCTKLDPGYSSGWYSNAYAHADYWGQGTQVIVSS

SEQ ID NO: 249 - protein, 126 aa, synthetic construct
EVQLVESGGGLVKPGGSLRLSCAASGFSLSTAWMNWVRRAPGKGLEWVGRIKIKTDGGTADYAAPVKGRFTISRDDSKNRVFLQMNSLKTEDTGLYYCTTDIYYYDFWSYYGMDVWGQGTTVTVSS

SEQ ID NO: 250 - protein, 124 aa, synthetic construct
EVQLVESGGGLVQPGGSLRLSCAASGFTFSSYWMHWVRQAPGKGLEWVSRISSGGISTSYADSVKGRFTISGDSAKNTVYLQMNSLRAEDAAVYYCARERPNLLWFGEYYFDYWGQGTLVTVSS

SEQ ID NO: 251 - protein, 115 aa, synthetic construct
QVQLVQSGAEVKKPGASVKVSCKTSGYTFTTFAMHWLRQAPGQRLEWMGWINAGNGLTKYSQKFQGRVTFTSDTSASTVYLELRSLRSEDTAVYFCGLRGGKDAWGQGTLVTVSS

SEQ ID NO: 252 - protein, 120 aa, synthetic construct
QVQLVESGGGVVQPGTSLRLSCAASGFTFSSYAIHWVRQAPGEGLEWLAVMSFDRSNKYADSVKGRFIISRDDSKNTVFLQMNRLTSEDTAVYYCAKDVTADLNWFLDFWGRGTLVTVSS

SEQ ID NO: 253 - protein, 122 aa, synthetic construct
QVQLHQSGPGLVQPSQTLSLTCVISGDSISSNSIGWTWIRQSPSRGLEWLGRIFYRSKWSTDYTDSVRGRMVIIPDASKNQLSLQLNSVTPEDTAVYYCARSRLLGRGLDYWGQGILVTVSS

SEQ ID NO: 254 - protein, 113 aa, synthetic construct
EVQLVESGGGLVQPGGSLRLSCAASGFTFSTSWMSWVRQAPGKGLEWVANINQDGTEKYYGDSVKGRFIISRDNAKNSLYLQMKSVRAEDTAVYYCVAGSSPWGQGALVTVSS

SEQ ID NO: 255 - protein, 123 aa, synthetic construct
EVQLLESGGGLVQPGGSLTVSCAASGFTFSRYAMSWVRLAPGKGLEWVSAISGSGETVYYADSVKGRFTVSRDNSKSTLFLQMHSLRGDDTALYYCARFPGVFKWFGDAFDLWGRGTRVTVSA

SEQ ID NO: 256 - protein, 115 aa, synthetic construct
EVQLLESGGDLVEIGGSLRLSCAASGFSFSGYAMSWVRQAPGQGLEWVAIIYSDGTSTFYADSVEGRFIISRDNSAETLFLQMNRLTVEDTAIYFCARGFYFDSWGQGTLVAVSS

SEQ ID NO: 257 - protein, 127 aa, synthetic construct
QVQLQESGPGLVKPSETLSLTCSVSGGSVGIPSYYWSWIRQPPGKGLEWIGFMYYTGSSNYNPSLKSRVTISLDMSNNQFSLKLSSVTAADTAVYYCARESATTTWYRVGLYGLDVWGHGTTVTVSS

SEQ ID NO: 258 - protein, 118 aa, synthetic construct
QVTLKESGPALVKPTQTLTLTCTFSGFSLTTSGMRVSWVRQPPGKALEWLARIDWDDDKFYRTSLKTRLTISKGTSKNQVVLTMTNMEPADTATYYCARTVDSQFDPWGQGTLVTVSS

SEQ ID NO: 259 - protein, 126 aa, synthetic construct
QVQLHESGPGLVKPSQTLSLTCTVSGASISSGGYYWTWIRQHPGKGLEWIGYIYYRGSTYYNSSLRSRVTISVDATDNQFSLELRSVTAADTAVYYCARGGITSGWYVVGNWFDPWGQGTLVTVSS

SEQ ID NO: 260 - protein, 120 aa, synthetic construct
QVQLVQSGAEVKKPGASVKVSCKASGYTFTAYNMYWVRQAPGQGLEWMGWIDPSSGGTNYAQKFQGRVTMTRDTSFNTVYMELSSLRSDDTALYFCARVGQWYPYYMDVWGQGTTVIVSS

SEQ ID NO: 261 - protein, 122 aa, synthetic construct
QVQLVQSGAEVKKPGSSVTVSCKASGDTITRYPITWVRQAPGQGPEWMGGIMPVYGRTEYAQKFQGRVTISADKSTNTVSMTLRIEDTAVYFCARMEHDSGWYPTGEYFRDWGQGTRVTVSS

SEQ ID NO: 262 - protein, 127 aa, synthetic construct
EVKLLESGGGLVQPGGSLRLSCGASGFTFSYYAMSWVRQAPGKGLEWVSYISPSGDATQYADSVKGRFTISRDNSKNTLYVQMNSLRVEDTAAYYCARVQSKGSSGWYWSGYYFDSWGPGTLVTVSS

SEQ ID NO: 263 - protein, 122 aa, synthetic construct
EVQLLESGGGLVQSGGSLRLSCAASGFTFSSYWMHWVRQASGKGLVWVSRISTDGSSTTYADSVKGRFTISRDNAQNTLYLQMNSLRAEDTAVYYCARGDSYYFGSGSVRDWGQGTLVTVSS

SEQ ID NO: 264 - protein, 123 aa, synthetic construct
QVQLQESGPRMVKPSETLSLTCTVSGDSISSYNYYWTWIRQSPGKGLEWIGSLHYGGSTYHNPSLKSRVMISLDMSKNQLSLNLISVTAADAAVYYCARVWGDFGYGRFGIDWGQGALVAVSS

SEQ ID NO: 265 - protein, 125 aa, synthetic construct
LLQLQESGPGRVKPSETLSLTCTVSGGSITTKSYYWSWIRQPPGKGLEWIGSIYSSGGTHYNPSLKSRITTSVDASRNQVSLQLKFVTAADTAIYYCARQTISGNRLIIAYFDTWGQGTQVIVSS

SEQ ID NO: 266 - protein, 121 aa, synthetic construct
QEQLVQSGAEVKKPGDSVKVSCQASGYTFTGYYIHWVRQSPGQGLEWMGRINPNTGGTNYAQRFQGRVTMTRDTSISTVYMELSRLTSADTAVYYCARGRGYSYGDVFDIWGQGTMVTVSS

SEQ ID NO: 267 - protein, 133 aa, synthetic construct
QTQLVQSGAEVKKTGSSLKVSCKASGYSISYRYLHWVRQAPGQALEWMGCITPFNGDTTSAQKFQDRVTITWDRSLSTVYMDLSSLRPEDTAMYYCASAPLKETSDSWYRGTYYYYYYGLEVWGQGTPVSVSS

SEQ ID NO: 268 - protein, 119 aa, synthetic construct
QMLLVESGGDLVKPGGSLRLSCAASGFTFTDYFMTWIRQAPGKGLEWVAYVSSSSLYINYAYSVTGRFTISRDNAKNLVFLQMTSLRAEDSAIYYCARGHFDQFHFDYWGQGTLVTVSS

SEQ ID NO: 269 - protein, 119 aa, synthetic construct
QMSLVESGGGLVRPGGSLRLSCAASGFTFTDYLMAWIRQAPGKGLEWVAYVSSSSLYVNYGFSVTGRFTISRDNAKNSLFLQMTSLRAEDSGIYYCAGGRFDQFHFDYWGQGTPVSVSS

SEQ ID NO: 270 - protein, 121 aa, synthetic construct
QITLKESGPTLVKPTQTLTLTCTFSGFSLSTPNMGVGWIRQPPGKALEWLAVIYWNGDLRYSPSLRSRLTLTKATSESQVVLAMTNMAPADTGTYYCARSIVTSDNWFDPWGQGALVTVSS

SEQ ID NO: 271 - protein, 123 aa, synthetic construct
EVQVVESGGGLVQPGGSLRLSCAASGISVSDNYMTWVRQAPGKGLEWVSLISRGGDTFYADSVKGRFTISRDSFKNTLYLQMNSLRADDTAIYFCATDGSAHSYQDRDQDDYWGQGTLVTVSS

SEQ ID NO: 272 - protein, 124 aa, synthetic construct
DVQLVESGGGLVQPGGSLKLSCEASGFIFSAYPFHWVRQVSGKGLEWLGRIRTKPNNFAPVYAASVKGRFTISRDDSKKTAFLQMNSLKTEDTAIYYCTRSYYDTIGRYYFDYWGRGTLVTVSS

SEQ ID NO: 273 - protein, 114 aa, synthetic construct
QVQVVESGGGVVQPGKSLRLSCAASGFTFSHYAMHWVRQAPGKGLEWVAVLSYDGSNKYYADSVKGRFTISRDSSKNTLYLQMNSLRAEDTAVYFCARDNFDYWGQGTLVTVSS

SEQ ID NO: 274 - protein, 112 aa, synthetic construct
EVQLVESGGGLVQPGESLRLSCVASGFTFTNYWMHWVRQPPGKGLEWVSRIDDGSGTSYATSVQGRFTISRDNSRNTVYLQMNSLRAEDTAVYYCTTVFEFWAQGAPVTVSS

SEQ ID NO: 275 - protein, 123 aa, synthetic construct
EVQLVESGGGLVKPGGSLRLSCAASGFSFNYAWMNWVRQSPGKGLEWVGRIKSKTDGGTIDYAASVKGRFTISRDDSENTLYLQVNSLNTEDTAVYYCTTDLTYTGSGGIDNWGQGTLVTVSS

SEQ ID NO: 276 - protein, 123 aa, synthetic construct
EVQLVESGGGLVQPGGSLRLSCAASGFTFSDSVMHWVRQASERRLEWVGHIETKANTYATAYAASVKGRFTISRDDSKNTAYLQMNSLKTEDTAVYYCTRHASYSGSQSGDYWGQGTLVTVSS

SEQ ID NO: 277 - protein, 117 aa, synthetic construct
QVHLVQSGAEVKKPGASVIVSCTTSGYTFTSYGISWVRQAPGQGLEWMGWISAYRGTTNYAQKFQGRVTLTKDTSASTAYMELRSLTSDDTAIYYCARDVDHDLDNWGQGTLVTVSS

SEQ ID NO: 278 - protein, 126 aa, synthetic construct
EVQLVESGGGLVQPGESLKISCAASGFSFSDSAVNWVRQAPGKGLEWVGRIRNKARNDATIYAASVEGRFTISRDDPKNTAYLQMNSLTIEDTATYYCTSRGPKDTNMDDVILDYWGQGTLVTVSS

SEQ ID NO: 279 - protein, 118 aa, synthetic construct
QLQLQESGPGLVKPSETLSLTCTVSDDSVSSSSYYWGWIRQPPGKGLEWIGNFYYGGNPYFNPSLKSRLSMSGDTSKNQFSLRLSSVTAADTAVYYCARFSNWVFDYWGQGTLVTVSS

SEQ ID NO: 280 - protein, 122 aa, synthetic construct
QVQLVQSGAELKKPGASVKVSCRASGYTFTSYALHWVRQAPGQRLEWMAWINAGNGITKYSQKFQDRVTITRDTSASIGYMELSSLRSEDTAVYYCARPGIGIGLFNALDIWGQGTPVTVSS

SEQ ID NO: 281 - protein, 126 aa, synthetic construct
EMQVVESGGGLVQPGGSWRLSCAASGFSVSNNYMMWVRQAPGKGLEWVSIIHGNGRPDYAESVKGRFTISRDNSKNTLHLQMNSLRPEDTALYFCARYCTDISCPDMVITTAIAICGQGTMVTVSS

SEQ ID NO: 282 - protein, 120 aa, synthetic construct
EVHLVESGGDLVQPGGSLRLSCEASGFTVSSYYMSWVRQAPGKGPEWVSSIYKGGGTFYADSVKGRFAISRDIGKNTVFLHMNILRAEDTAVYFCASTLDSGNTYYFDSWGQGTLVTVSS

SEQ ID NO: 283 - protein, 122 aa, synthetic construct
QVHLVQSGAEVKKPGASVKISCKASGYTFTTYSIHWLRQAPGQSLEWMAWINAGNGNTKYSQKFQGRVTITRDTSATTAYMELSNLRFEDTALYYCAKDPGYNYGGRWFDPWGQGTLVTVSS

SEQ ID NO: 284 - protein, 114 aa, synthetic construct
QVLLVESGGGVVQPGRSLRLSCAASGFTFRSYGMHWVRQAPGKGLEWVAVIAFDGSEKYYADSVKGRITISRDNSKNTVYLQMNSLRAEDTAVYYCAKLDMDVWGQGTTVTVSS

SEQ ID NO: 285 - protein, 125 aa, synthetic construct
QVHLVESGGGLVKPGGSLSLSCAASGITVSDYHMTWIRQAPGKGLEWVAYSSSSGSNTNYADSVKGRFTISRDNAKNSLYLQMNSLRNEDTAVYYCARLFFVYYDFPRENGMDVWGQGTTVTVSS

SEQ ID NO: 286 - protein, 130 aa, synthetic construct
QLQLQESGPGLVKPSETLSLTCSVSGGSISGTSHFWAWIRQPPGKGLEWIGSIYWSGATYYNPSLKSRVTMDVDTSKKQFSLKLSSVTAADTAVYYCASQSYDIQTGWAAFFYYYHIAVWGKGTTVTVSS

SEQ ID NO: 287 - protein, 121 aa, synthetic construct
QVKLEESGGGVVQPGRTLRLSCVVSGFTFSNYGMHWVRQAPGQGLEWVALIWKDGTNKYYAESVQGRCTISRDNSKNTLYLRLNTVRDEDTAIYYCAREDREVVPAAFDYWGQGALVTVSS

SEQ ID NO: 288 - protein, 114 aa, synthetic construct
QEHLVEAGGGVVQPGKSLRLSCAASGFSFSRYGIHWVRQAPGKGLEWVAVISYDGTNRYYADSVKGRFTISRDNSKKTVYLQINSLRVEDTAVYFCSKLEMDYWGRGIQVTVSS

SEQ ID NO: 289 - protein, 123 aa, synthetic construct
EVQLAESGGGLVKPGGSLRLSCAASGFALSRYSMNWVRQAPGKGLEWVSSISSSSNYIFYGDSVKGRFTISRDNAKNSLYLQMNGLRAEDTAVYYCARWVTAATSYYYYMDVWGKGTTVTVSS

SEQ ID NO: 290 - protein, 122 aa, synthetic construct
QVQLVQSGAEVMKPGASVKVSCKASGYTLNNYDVNWVRQASGQGPEWMGWMRPGGDTGFAHKFQGRVSITRDTSINTAYMELSSLRSEDTAVYFCARGSAGGQSYFNGLDVWGQGTPVIVAS

SEQ ID NO: 291 - protein, 124 aa, synthetic construct
QITLKESGPTLVKPTETLTLTCTFSGSSVSTSGLGVGWIRQAPGKALEWLAISYWNHDQRYNPSLMSRLTTTKDDSRDQVVLTMTNMDPMDTGTYFCAHRRLSSLKSAGYYDHWGPGILVTVSS

SEQ ID NO: 292 - protein, 126 aa, synthetic construct
QVQLVQSGSELKKPGASVKVSCKASGYSFTNYAIDWVRQAPGQGLEWMGWINTNTGNPTSAPGFTGRFVFSLDTSVRTAYLQISNLKAEDTAIYYCARESLVAAGGHYQYNGMDVWGQGTTVTVSS

SEQ ID NO: 293 - protein, 120 aa, synthetic construct
QVQLVESGGGVVQPGRSLRLSCEASGFSFSNYGLQWVRQPPGKGLEWVAFILNDGRTKNYVDSVKGRFTISRDNSKNTLYLEMDSLRAEDTAVYYCARHKFGDGYGMDVWGQGTTVIVSS

SEQ ID NO: 294 - protein, 116 aa, synthetic construct
DVQLVESGGGLVQPGGSLRLSCTASGFTFSTYWMTWVRQIPGKGLEWVATIERRGDKKHYVDSVKGRFTISRDNAQSSLYLQMDSLRVDDTAMYYCVRENWVFDYWGQGVLVTVSS

SEQ ID NO: 295 - protein, 122 aa, synthetic construct
QVQVVESGGGVVQPGRSLTLSCAGSGFTFNSYAIHWVRQAPGKGLEWVAVSVSSDGHYKYYADSVQGRFTISTDSSTSTLYLHMNSLRVEDTALYYCARAWGLSRGVIIGYWGQGTLVTVSS

SEQ ID NO: 296 - protein, 126 aa, synthetic construct
QDQLEQSGAELKKPGASMKVSCKFSGDSFTRFAIHWLRQAPGQGLEWMGWINTGNGLTKYAPRFQGRLSITRDTSANTAYMELTSLTSTDTAVYYCARATDCSGPRCFVLGWYDPWGQGTLVTVSS

SEQ ID NO: 297 - protein, 113 aa, synthetic construct
QVQLVESGGGVVQPGRSLRLSCAASGFTFSRYGMHWVRHAPGKGLEWVAAISYDGRNKYYGDSMKGRFTLSRDNPKNMVYLQMNSLKPDDTGVYYCAGDLAHWGQGTLVTVSS

SEQ ID NO: 298 - protein, 119 aa, synthetic construct
EVQVVESGGGLVQPGLSLRLSCTASGFPFGAYAMSWVRQAPGKGLEWVSLIRIKTSGGTKHYAASVEGRFTISRDDSRGILYLHMDSLKTEDTAVYYCARGFTNPFDYWGQGTPVTVSS

SEQ ID NO: 299 - protein, 123 aa, synthetic construct
DVQLVESGGGLVQPGGSLRLSCAVSGFTFSDHYIDWVRQAPGKGPEWVGRNRNKANNYSTEYAASVRGRFTVSREESTTSVHLQMSSLRTDDTAMYYCVRVYAEPGKYYFDFWGQGTQVTVSS

SEQ ID NO: 300 - protein, 130 aa, synthetic construct
QLQLQESGPGLVKPSETLSLTCTVSGGSISRSSNYWGWIRQPPGKGLEWLGSIYFSGTTNYNPSLKSRVTISVDTSKNQFSLNLNSVTAADTAVYYCASQSYSRSSGWALIYYYYFMDVWGKGTTVTVSS

SEQ ID NO: 301 - protein, 120 aa, synthetic construct
QVQLQQSGPGLVKPSQTLSLTCVVSGDSLFSNGVAWNWFRQSPSRGLEWLGRTYYRSKWYNEYEVSVKSRIMIDPDTSKIQFSLQLNSVTPEDTAVYYCARGRNNAFDFWGQGTTVTVSS

SEQ ID NO: 302 - protein, 115 aa, synthetic construct
EVQVLESGGGLVKPGGSLRLSCATSGFPFSNAWMNWVRQAPGKGLEWVGRIKSKTDGGTPDYAAPVKGRFTISRDDSKNRLYLQMNSLKTEDTAVYYCTTAFDYWGQGTLVTVSS

SEQ ID NO: 303 - protein, 113 aa, synthetic construct
QVQLVESGGGVVQPGRSLRLSCVGSRFTFSRYGMHWVRQAPGKGLEWVAVVSYDGSNKYYVDSVKGRFTISRDNSKNTLYLQMNNLRAEDTAVYYCAKDLDVWGKGTTVTVSS

SEQ ID NO: 304 - protein, 116 aa, synthetic construct
QVQLQQPGPRLVKPSQTLSLTCAISGESVSSNTAAWNWIRQSPSRGLEWLGRTYYRSKWITDYAVSLRSRMTINSDTSNNQVSLHLTSVTLEDTAVYYCAYGLDVWGQGTTVIVSS

SEQ ID NO: 305 - protein, 120 aa, synthetic construct
EVQLFESGGGLVQPGGSLRLSCAASGFTFSTCGMSWVRQSPTKGLEWVSSISASGDLTFYADSVKGRFTISRDNSKNTLFLQINNLRADDTAIYYCAKIGVIARWYYDFWGRGNLVTVSP

SEQ ID NO: 306 - protein, 123 aa, synthetic construct
QVQLVESGGGVVQPGRSLRLSCAASGFTFSSYGMHWVRQAPGKGLEWVALIWYDGSNKYYADSVKGRFTISRDNSKNTLYLQMNSLRAEDTAVYYCARVRDTSGYHGDAFDIWGQGTMVTVSS

SEQ ID NO: 307 - protein, 126 aa, synthetic construct
QEQLVESGGGVVQPGRSLRLSCAASAFTFSSHGMHWVRQAPGKGLEWVAIIWFDGSQKYYRDSVKGRFTISRDNSKNTLFLQMNSLRAEDTAVYYCARTRQGEDYDSLIGDAFDLWGQGTMVTVSS

SEQ ID NO: 308 - protein, 129 aa, synthetic construct
EVQLLESGGGLVQPGGSLRLSCAASGFTFNNYAMTWVRQAPGKGLEWVSIISASGDSTNYADSVKGRFTISRDNSKKKLYLQMNSLRAEDTAVYYCAKVVPEYNNGWYSYYYGYMDVWGTGTTVSVSST

SEQ ID NO: 309 - protein, 127 aa, synthetic construct
QVLLQQWGAGLLKPSETLSLTCADYGGSFSYYYWTWIRQPPGKGLEWIGEINHSGSTNYSPSLKSRVTISVDASKSRFSLRLTSVTAADTAVYYCARGRPPYISGWRIPGKYFFDYWGQGALVTVSS

SEQ ID NO: 310 - protein, 116 aa, synthetic construct
EVQLLESGGGLVQPGGSLRLSCEASGFTFSATGMTWVRQAPGKGLECVSAIGVNVANTDYADSVKGRFTIFRDNSKNTLYLQMSSLRAEDTAVYYCAKGTTNIDYWGQGIQVTVSS

SEQ ID NO: 311 - protein, 120 aa, synthetic construct
EVQLVESGGGLVQPGGSLRLSCAASGFSFSSHYMAWVRQAPGKGLEWVGNIRQDASAAFYVDSLKGRFTISRDNAKNLLFLQLNSLRAEDTAVYYCARWTYSSVWFLDYWGQGTLVTVSS

SEQ ID NO: 312 - protein, 125 aa, synthetic construct
QVQLVQSGAEVKKPGSSVKVSCEASGGGFSNFGISWVRQAPGQGLEWMGRIVPLVHITDYAQKFQGRVTITADRSTSTVYMELRSLRSEDWAVYYCARSKYQLLYVARTDLLDNWGQGTLVKVSS

SEQ ID NO: 313 - protein, 121 aa, synthetic construct
QVHLVESGGGVVQPGRSLRLACAASGFTFSSYGLHWVRRAPGKGLEWVALIWSDGSNHYYGDSVKGRFTISRDNLKNTLYLQMNSLRAEDTAVYYCAREKRIAAPAAFDYWGQGTLVTVSS

SEQ ID NO: 314 - protein, 120 aa, synthetic construct
QVQLQESGPGLVKPSETLSLTCTVAGGSISTYYWSWIRQSAGKGLEWVGRIKSNGNTHYNPSLKSRVTMSIDSSKNQVSLKMSSVIAADTAVYYCARLNLPASRGAFDIWGQGTMVTVSS

SEQ ID NO: 315 - protein, 122 aa, synthetic construct
EVQLQQSGGEVKKPGESLRISCQGSGYTFSNYWIGWVRQMPGKGLDLVGLIYPRDSDTRYSPSFQGRVTISADKSVSTAFLQWSSLQASDSAMYFCARGLGGGTYGGDFDSWGQGTLVTVSS

SEQ ID NO: 316 - protein, 126 aa, synthetic construct
QVQLQESGPGLVKPSQTVSLTCTVSGGSISSRSNYWSWIRQPAGKGLEWIGHIYTSGNTNYNPSLKSRVTISRDTSKNQFSLKLSSVTASDTAVYYCARDLGLSSNWDIHWYFDLWGRGTLVTVSS

SEQ ID NO: 317 - protein, 128 aa, synthetic construct
QVQLVQSGAEVREPGSSVKVSCKAPGGIFSYYAFSWVRQAPGQGLQWMGGLVPALGATTYAQRFRDRVTITADASSRTVYMELRNLRSEDTAIYYCARDQGELATIAPYYSYYTLDVWGQGTTVTVSS

SEQ ID NO: 318 - protein, 123 aa, synthetic construct
EVQLVESGGGLVQPGGSLRLSCVCSGFTFSNYWMTWVRQAPGKGLEWVANIKKDGSEKYYVDSVRGRFAISRDNAKTSLYLEMNSLRVEDTAVYYCRSLLDPAYNYGRGDGRWGQGTLVTVSS

SEQ ID NO: 319 - protein, 121 aa, synthetic construct
QVQLVESGGGVVHPGRSLRLSCAASGFSFSRYGMHWVRQAPGKGLEWLALTWHDGSNKHYADSAKGRFTIYRDNSKNMLFLQINSLRVEDTAVYYCAREVPEVAMAVLDYWGQGTLVTVSS

SEQ ID NO: 320 - protein, 121 aa, synthetic construct
QVQLQESGPGLVKPSGTLSLTCAVSGVSISGSHWWSWVRQSPGKGLEWIGEIFHAGLTNYNLSLKSRVTMSVDKSKNQFSLTLTSVTAADTAVYYCARVMVWITATHFDSWGQGTLVSVSS

SEQ ID NO: 321 - protein, 122 aa, synthetic construct
EVQLVESGGGLVQPGGSLRLSCAASGFTLSDHYMDWVRQAPGKGLEWVGRIRTQVRSYDTDYAASVKGRFTVSRDDSRNSVYLQMNSLKTEDTAVYYCVRIGPIGYDGMDVWGQGTTVTVSS

SEQ ID NO: 322 - protein, 126 aa, synthetic construct
QLQLQESGPGLVKPSETLSLTCSVSSASISSTSSYWAWIRQPPGKGLEWIGSIYYSGNTFYNPSLRSRLTISVDTSKKQFSLTLRSVTAADTAVYYCARQSLYTRSWYIESYFDYWGQGTVVTVSS

SEQ ID NO: 323 - protein, 126 aa, synthetic construct
QLQLQESGPGLVKPSETLSLTCTVSGGSISSMSHYWGWIRQPPRKGLEWIGSIYYSGTTYYNPSLKSRVTISVDTSKNQFSLRLSSATAADTAIYYCARQVAYRSGWYVYYYVDVWGKGTTVTVSS

SEQ ID NO: 324 - protein, 121 aa, synthetic construct
QVQLQESGPGLVKPSETLSLTCSVSSGFISNYYWGWIRQPPGKGLEWIGYIHHNGRSDYSPSLKSRVSILLDTPKNQISLRLTSVTAADTAMYYCAGDRYSAYNPSAFEIWGHGTKVTVSS

SEQ ID NO: 325 - protein, 128 aa, synthetic construct
DVQLLESGGGLVQPGGSLRLSCEASGFTFNIFGMSWVRQTPGKGLEWVSGISPSGDSTYYSDSVKGRFTISRDNSKHTVYVQMKSLRAEDTAVYYCAKTSPQYINGWYAKWINYFDYWGRGTLVTVSS

SEQ ID NO: 326 - protein, 120 aa, synthetic construct
QVQLVESGGGVVQPGRSLRLSCEVSGFFFSRYGMHWVRQAPGKGLEWVGCIWHDGSNQYYGDSVKGRFTISRDNSKNTFYLQMNRLRVDDTAVYYCAREGTTAVTIYDYWGQGTLVTVSS

SEQ ID NO: 327 - protein, 116 aa, synthetic construct
EVQLVESGRNLVRPGGSLRLSCAASGFGFTTHAMSWIRQAPGKGLEWVSLITGNGETTAYIDSVKGRFTISRDNSKNMLYLQMNSLRVEDTAIYYCVKGYGLFDFWGQGTLVTVSS

SEQ ID NO: 328 - protein, 119 aa, synthetic construct
QVQLVQSGAEVKKPGASVRISCKASGHTFNAFHIHWVRQAPGQGFEWMGKFFSSGDQTRFPQKFQGRITMTRDTSTSTAYMELTSLTSDDTAVYYCAREGPFTYYFDYWGQGTLLTVSS

SEQ ID NO: 329 - protein, 123 aa, synthetic construct
EVQLLESGGGLVQRGGSLRLSCAASQFAFSKYTMTWVRQAPGKGLDWVAAISGSSEHTYYADSVKGRFTISRDNSKNTVFLQMSSLRADDTAVYYCAKDGVMVSATPWGMDVWGRGTTVIVSS

SEQ ID NO: 330 - protein, 117 aa, synthetic construct
QLLLQESGPGLVKPSETMSLTCSVSGDSLSSDFNFWGWTRQSPGKGLEWIGSISPRGATYYNPSLQSRVTMSIDTSKNELSLKLTSVTAADTAVYLCVKGHGNISKWGQGTLITVSS

SEQ ID NO: 331 - protein, 123 aa, synthetic construct
QVHLVQSGGEVKRPGASVKVSCKASGYTFNNHGVSWVRQAPGQGLEWMGWVSTHKSNTNHAQRFQGRVTMTTDTSTTTAYLEVRSLRSDDTAVYYCATGRLFGVTIPDAFDIWGQGTLVTVSS

SEQ ID NO: 332 - protein, 127 aa, synthetic construct
QAQLMQSGAEVKKPGASVKVSCKASGYSFTSYGISWVRQAPGQGLEWMGWISTYNGNTDYAQTLQGRVTMTTDTSTSTAYMELRSLRSDDTAVYYCVRVVAYCIGGSCYAVGYFDYWGQGTPVTVSS

SEQ ID NO: 333 - protein, 114 aa, synthetic construct
QVQLVESGGGVVQPGRSLRLSCAASGFTFNSYGMHWVRLAPGKGLEWVAVISYDGTKKYYADSVKGRFSISRDNSKNTLDLQMNSLRLEDTAVYYCAKLDFDYWGQGTLVTVSS

SEQ ID NO: 334 - protein, 123 aa, synthetic construct
QVQLVQSAAEVKKPRSSVKVSCKASGGTFGNNAISWVRQAPGQGLEWMGGIVPFLTTVNYAQKFQDRVTITADRSTNTAYMELVRLTSEDTAVYYCARVRRSGYNSVYYFDMWGQGTLVTVSS

SEQ ID NO: 335 - protein, 130 aa, synthetic construct
QLHLQESGPGLVKPSETLSLTCTVSGGSIRSSSYYWGWLRQPPGKDLEWIGSIYSSGTTYYSPSLESRVTVLVDTSKNQFSLRLTSVTAADTAVYYCARPVHLDYSTISYYPGNGPFDIWGPGTLVTVSA

SEQ ID NO: 336 - protein, 130 aa, synthetic construct
EVQLVGTGGGLVQPGRSLTLSYAASGFNFGDYTMSWVRQAPGKGLEWLGLIREKPRGGAAEYAASVRDRFTISRDDSKSIAYLQMSSLRTEDTAVYYCIPLAWSPRDGYNGIFDSWGQGTLVTVSEGVSA

SEQ ID NO: 337 - protein, 127 aa, synthetic construct
EVQLVESGGGLVQPGRSLRLSCTASGFTFGDYVMSWVRRAPGKGLEWLGFIRSKLYGGTPEYAASVKGRVTISRDDSRGIAYLQMNSLKTEDTAFYYCTRLRLYRSGWYIEYYFDYWGQGTLVTVSS

SEQ ID NO: 338 - protein, 123 aa, synthetic construct
EVQLLQSGGGLVQPGGSLRLSCAASGFSFNNYAMTWVRQAPGMGLEWVSSTTNSGGRTYYADSVKGRFTISRDNSKNTLFLQMNSLRAEDTAVYYCAKDGVRASYDPWGSDYWGQGTLVTVSS

SEQ ID NO: 339 - protein, 129 aa, synthetic construct
EVKLVESGGGLVKPGGSLRLSCAGSGFTFNRYNMNWVRQAPGKGLEWVSSISSSSSYKHYAVSVEGRFTVSRDNARNSVYLQMNSLRDEDTAVYYCARPLFEITMVRGVLMTVDAFDIWGQGTMVTVSS

SEQ ID NO: 340 - protein, 123 aa, synthetic construct
QEQLVQSGTEVKKPGASVKVSCKTSGYTFTDYGVSWVRQAPGHGLEWMGWIRPYDGNTNYAQKVQGRVTMTTDTSTSTAYMELRSLRSDDTAIYYCARLSGDGRFYHGMDVWGQGTTVTVSSP

SEQ ID NO: 341 - protein, 126 aa, synthetic construct
QLQLQESGPGLVKPSETLSLTCTVSGGSISSLSHYWGWVRQPPGKGLEWIGSIYYSGTTYYKPSLKSRVTISVDTSRNQLSLKLNFVTAADTAVYYCARLTGYRDGWYIYWYFDLWGRGTLVSVSS

SEQ ID NO: 342 - protein, 120 aa, synthetic construct
QVQMVQSGAEVKKPGASVKVSCKASGNTFTAHYLHWVRQAPGQRFEWMGWINPNSGGTYYPQKFQGRVTMTRDTSFTTVYMELRRLRSDDTAVYYCARESGAGSTHFDYWGQGTLVTVSS

SEQ ID NO: 343 - protein, 122 aa, synthetic construct
EVQLEESGGGLAQPRGSLRLSCVGSGLTFSTYTMNWVRQAPGKGLEWISYISTNGATIHYADSVKGRFTISRDNAKNSLYLHMNSLRDDDTAVYHCATTGFGSGSYWYFDLWGRGTLVTVSS

SEQ ID NO: 344 - protein, 122 aa, synthetic construct
QIQLQQSGPGLVKPSQTLSITCAISGDSVSNNHAGWNWIRQSPSRGLEWLGRTYYRSRWYNDYAVFVESRITITADTFKNEVSLQLTSVTPEDTAVYYCTRVKELGRGFQYWGQGSLITVSS

SEQ ID NO: 345 - protein, 121 aa, synthetic construct
AVQLVESGGGVVRPGGSLRLSCVASGFTFDDFAMSWVRQVPGKGLEWVSGLNSEGLKSGYADSVRGRFTISRDNAKNSLYLEMNSLRVEDTAFYYCARGASGGWYFDSEFWGQGALVTVSS

SEQ ID NO: 346 - protein, 123 aa, synthetic construct
QVQLVQSGPEMKKPGASVKISCKASGYQNNNYAIYWVRQAPGQRPEWMGWINAGNGNTQYSQRFRGRVTITTDASTRTSYMDLSGLRSADTAVYYCASHTPNSVTAYDPFHYWGQGTLVTVSS

SEQ ID NO: 347 - protein, 127 aa, synthetic construct
QVQLQASGPGLVKPSETLSLTCSVSGDSIRSSSFWGWIRQPPGKGLEWIGSIYFSGSTYHNPSLKSRVTMSVDMSRNQFSLMLSSVTAADTAVYYCARQRFGSGWALFYYYYYMDVWGQGTTVTVSS

SEQ ID NO: 348 - protein, 123 aa, synthetic construct
EVQLVESGGGFVQTGGSLRLSCVASGFTFSDYEMNWVRQAPGKGLEWVSYISSGGGTTKYAESVKGRFTISRDNAKNSVFLQMHSLRADDTAIYYCARESALQFNTILFFDFWGQGTLVTVSS

SEQ ID NO: 349 - protein, 121 aa, synthetic construct
QVQIVQSGAEVKKPGASVKVSCKTSGYTFTSRNMHWVRQAPGQRPEWVGWINPDSGDTKYAQWLQDRVTLSRDTSASTAYMDLRSLRSEDTATYYCVREAGGGTESYFVHWGQGTLVTVSS

SEQ ID NO: 350 - protein, 129 aa, synthetic construct
EMQLLESGGGLVNPGGSLRVSCAASGFTFSQAWMSWVRQAPGKGLEWVGRIESTTDGGTTQYAAPVKGRFTISRDDSKSSLFLQMNSVRAEDTAVYYCTTAFGNYNKGHVYVYPYMDVWGKGTTVTVSS

SEQ ID NO: 351 - protein, 121 aa, synthetic construct
GVQLVQSGAEVKKPGATVKISCKISGYTFIDYHMHWVKQAPGKGLEWMGLVDPEDGERIYAKKFQGRVIITADTSTDTAYMELSSLRSDDTALYYCATFEFGGSDAGGFWGQGTLVTVSST

SEQ ID NO: 352 - protein, 129 aa, synthetic construct
QVQLVESGGGVVQPGRSLRVSCAASGFSFSTYGMHWVRQAPGKGLEWVALIWYDGSSKYSADSVKGRFTISRDNSKNTLFLQMNRLRADDTAVYYCARDLFPTIRGISRDHHYYGMDVWGQGTTVTVSS

SEQ ID NO: 353 - protein, 128 aa, synthetic construct
EEQLLDSGGGLVQPGGSLRLSCAASGFTFRNYAMTWVRQAPGKGLEWVSSITTTGDRATYADSVKGRFTISRDNSKNTLFLQMNSLRVEDTAVYYCAKDFPRYNSGWYANDHNYFGSWGQGTLVTVSS

SEQ ID NO: 354 - protein, 119 aa, synthetic construct
QVQLVESGGGVVQPGTSLRLSCATSGFTFSKYGMHWLRQAPGKGLEWVAVISYDGNHKYYSDSVKGRFTISRDNSKNTLYLQLNSLRGEDTAVFFCAKLAGTTHSFAYWGQGTLVTVSS

SEQ ID NO: 355 - protein, 126 aa, synthetic construct
QLQLQESGPGLVKPSETLSLTCTVSGGSIRSISDWWGWIRQPPGKGLEWIGNIYYTGSTNYSPSLRSRVIISADMPKNQFSLRLSSVTAADTAVYYCARQVAYRDGWYVRWYFDLWGRGTRVTVSS

SEQ ID NO: 356 - protein, 122 aa, synthetic construct
QVQLVQSGSEVKKPGASVRVSCKASGYFFNYYYIHWVRQAPGQGLEWMGWINPNSGDTDSVKKFQGRVTMTRDTSISTAYMDLRSLTSVDTAVYYCARLNRGYTHGSALDFWGQGTIVTVSS

SEQ ID NO: 357 - protein, 117 aa, synthetic construct
QVQLVQSGAEVKKPGASVKVSCKTSGYSFTINGISWVRQAPGQGLEWVGWMSANSGNTIYAQKFQSRVTMTRNTSTSTAHMELRSLRSDDTAIYYCARDRNYALESWGQGTLVTVSS

SEQ ID NO: 358 - protein, 119 aa, synthetic construct
QVQLVESGGGVVQPGRSLRLSCAASGFTFSSYAMHWVRQAPGKGLEWVAIISYDGSKKYLADSVKGRFTISRDNSKNTLYLQMNSLRSEDTAVYYCASEPDTAYIVDHWGQGTLVTVSS

SEQ ID NO: 359 - protein, 130 aa, synthetic construct
QLQLQESGPGLVKPSETLSLTCSVSGGSMRSSSYYWAWIRQPPGKGLEWIGSIYNSGTTYYHPSLESRVTMSVDTSMNQFSLKLSSVNAADTAVYYCARPAAVDYSLTSYYPGEGAFDVWGQGTMITVSS

SEQ ID NO: 360 - protein, 122 aa, synthetic construct
EVQLVESGGGLVQPGGSLRLSCGASGFTFSNFAITWVRQAPGKGLEWVSVISGSGGRTYYADSLKGRFIISRDNSKNMVYLQMNSLRAEDTGVYYCAKNKNDKSGQYPLYYWGQGALVTVSS

SEQ ID NO: 361 - protein, 122 aa, synthetic construct
QVQLQESGPGLVEPSGTLSLTCAVSGASISNFHWWSWIRQPPGKGLEWIGQIHRDGSTKYNPNPSLKSRVTVSLDKSNNNFSLRLTSVTAADTAVYYCAAEDGSGTNWFDPWGQGNLVTVSS

SEQ ID NO: 362 - protein, 126 aa, synthetic construct
QVQLQESGPGLVKPSETLSLTCTVSGDSISSSNYYWGWIRQPPGKGLDYIGSIHNSGRTSYNPSLKSRVTVSVDTSKNQFSLKLTSVTAADTAVYYCARQGSYSSGWYPFDFEDYWGQGVLVTVSS

SEQ ID NO: 363 - protein, 127 aa, synthetic construct
QVQLVQSGAEVKKPGASVKVSCKASGYTFTIYDISWVRQAPGQGLEWMGWISAYNGHTNYAQKLQGRVTMTTDTSTSTAYMELRSLRSDDTAVYYCARGYRKSSSGWYWNNWYFDLWGRGTLVTVSS

SEQ ID NO: 364 - protein, 129 aa, synthetic construct
QVTLKESGPVLVKPTETLTLTCTVSGFSLNNARMGVSWIRQPPGKALEWLAHIFSNDEKSFSTSLKSRLTISKDTSKGQVVLTLTNMDPVDTATYFCARIPHRSGSYQPYYYYYGMDVWGQGTTVTVSS

SEQ ID NO: 365 - protein, 118 aa, synthetic construct
EVQLVESGGGAIQPGGSLRLSCVASGFRVNWNYMTWVRQAPGKGLEWVSVIHIDGTTYYADSVKGRFTISRDNSNNLLFLQMNNVRAEDTAVYFCARDTDSWWRFDFWGQGTLVTVSS

SEQ ID NO: 366 - protein, 124 aa, synthetic construct
QVQLVQSGAEVKKPGSSVKVSCKASGGTFSSYAISWVRQAPGQGLEWMGGIIPIFGVTKYTQKFQGRVTITADRSTNTAYMELSSLRSEDTALYYCASRTIVVALNYYYGMDVWGQGTTVTVSS

SEQ ID NO: 367 - protein, 128 aa, synthetic construct
EVQLLESGGGLVQPGGSLRLSCAASGFTFSFYAMSWVRQAPRKGLEWVSTISTSGDITYYADSVKGRFTISRDNSKNTLYLQMNSLRAEDTAVYYCATNTPLKSSGWYISGFGYFDYWGQGTLVTVSS

SEQ ID NO: 368 - protein, 127 aa, synthetic construct
QLQLQESGPGLVKPSETLSLTCTVSGGSISSSSHYWGWIRQPPGKGLEWIGRIYYSGSTSYNPSLKSRVTISIDTSKNQFSLKLNSVTAADSAVYYCARDMIYSSSWYLVAGNFDSWGQGTLVTVSS

SEQ ID NO: 369 - protein, 128 aa, synthetic construct
QVQLVQSGAEVKKPGASVKVSCKASGYSFSSYGISWVRQAPGQGLEWMGWISGYTGKTNYAQKFQGRVTMTTDTATSTAHMELRSLRSDDTAIYYCARAPHAKNNGWYVEGPGAFDFWGQGTTVTVSS

SEQ ID NO: 370 - protein, 117 aa, synthetic construct
DVQLVESGGGLVQPGESLRLSCTASGFTLSTYWMSWVRQAPGKGLEWVAKIKEDGSEKYYLDSVKGRFTMSRDNAKNSLDLQMNSLRAEDTAVYYCARDSTGGFDYWGRGTLVTVSS

SEQ ID NO: 371 - protein, 120 aa, synthetic construct
EVQMVESGGGLVQPGGSLRLSCAASGFTFSSYEMNWVRQAPGKGLEWVSYISSSGSTKYYADSVKGRFTISRDNAKNSLYLQMNSLRAEDTAVYFCARDTMLWTYYFDNWGQGTLVTVSS

SEQ ID NO: 372 - protein, 124 aa, synthetic construct
QVQLVQSGAEVKKPGASVKVSCKASGYTFTSYDIHWARQATGQRLEWMGWLSPNTGNTGYAQKFQGRVILTRNTSINTAYMEVTSLRFQDAAVYYCARGHTGYSNGWDSWLDPWGQGTLVTVSS

SEQ ID NO: 373 - protein, 135 aa, synthetic construct
QVQLVESGGGVVQPGRSLRLSCAASGFTFSSYIMHWVRQAPGKGLEWVAVISYDGSNKYYADSVKGRFTISRDNSKNTLYLQMNSLRAEDTAVYYCAREDPGRFGELLVQLGGGRNYYYYGMDVWGQGTTVTVSS

SEQ ID NO: 374 - protein, 127 aa, synthetic construct
QVQLVQSGAEVKKPGASVKVSCKASGYTFINYGISWVRQAPGQGLEWMGRINAYSGYTNLPQKLQGRVTMTTDTSTSTVYMELRSLRSDDTAVYYCARDAPTYSSGWYRHYYYMDVWGKGTTVTVSS

SEQ ID NO: 375 - protein, 127 aa, synthetic construct
EVQLVESGGGLVKPGGSLRLSCAASGFTFNTYNMNWVRQAPGKGLEWVSSISSSSSYINYAESVKGRFTISRDNVKKSLYLQMNSLRAEDTAVYYCAGQRITLGRGAITAAPYLDHWGQGTRVAVSS

SEQ ID NO: 376 - protein, 126 aa, synthetic construct
QLQLQESGPGLVKPSETLSLSCTVSGGSIKSPSFYWGWIRQPPGKGPEWIGSFYYSGTTFYSPSLKGRVTISTDTSKNQFSLRLSSVTAADTAVYFCAREFLYMSDLLVGQAFDIWGQGTMVTVSS

SEQ ID NO: 377 - protein, 122 aa, synthetic construct
EVQLVESGGGLVQPGGSLRLSCAASGFPFSAYTMSWVRQAPGKALEWISYISTTSDTIYYADSVKGRFTISRDNAKNSVSLQMKSLRVEDSALYFCARVAFGSGSYWFFDYWGQGILVTVSS

SEQ ID NO: 378 - protein, 123 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARPGDRGTDHTDAFDIWGQGTMVTVSS

SEQ ID NO: 379 - protein, 119 aa, synthetic construct
EEQLVESGGGLVQPGGSLRLSCAASGFTFSSYEMNWVRQAPGKGLEWVSDISSSGSTIYYADSVKGRFTISRDNAKNSLYLQMNSLRAEDTAVYYCARGELDLGAFDIWGQGTMVTVSS

SEQ ID NO: 380 - protein, 122 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCTAATQYGLLGGAFDYWGQGTLVTVSS

SEQ ID NO: 381 - protein, 127 aa, synthetic construct
EVQLVQSGGGQVQPGESLRLSCAASGFTFTSYWMSWVRQAPGKGPEWVANINHAGSQKYYVDSVKGRFTISRDNAKNSLELQMNSLRAEDTAVYYCARFWRVLPDYFDTSGRAVDYWGQGILVTVSS

SEQ ID NO: 382 - protein, 129 aa, synthetic construct
QVQLVQSGAEVKKPGASVKVSCKASGYSFTTHGISWVRQAPGQGLEWMGWISAYSGNTNYAQKLQGRVTMTTDTSTSTAYMELRSLRSDDTAVYYCARDKLLWFRELPHLPRDNWFDPWGQGTLVTVSS

SEQ ID NO: 383 - protein, 129 aa, synthetic construct
QVQLQQWGAGLLKPSETLSLTCAVYGGSFSDYYWTWIRQPPGKGLEWIGEINHSGRTNYKSSLRSRVTISVDTSKNQFSLKLTSVTAADTAMYYCARGQIQISMMAVIFTGGAYYFDSWGQGTLVAVSS

SEQ ID NO: 384 - protein, 126 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARPGFFSTYYEDRGDAFDIWGQGTMVTVSS

SEQ ID NO: 385 - protein, 126 aa, synthetic construct
QVQLQESGPGLVKPSGTLSLTCDVSGASITSTKNWCSWVRQSPGKGLEWIGEIYHSGDTNYNPSLKSRVTMSVDTSKNQFSLKLSSVTAADTAVYYCARDMRIPARPDQGHYFDFWGQGTLVTVSS

SEQ ID NO: 386 - protein, 118 aa, synthetic construct
AEQLLESGGGLVQPGESLRLSCAASGFTFGSYGLSWVRQVPGKGLEWVSHIRVRGSGTSYADSVKGRFAISRDNSNNMLYLQMSSLRAEDTAVYYCAKSTGWLQFDCWGQGTLVTVSS

SEQ ID NO: 387 - protein, 121 aa, synthetic construct
QVHLLELGPRLVKPSETLSLTCAVSGYSVGSGYYWGWIRQPPGKGLEWVGTIYFTENTSFGPSFGGRATISVDASENTFSLTLDSVTAADTAMYYCAARTAVVNEYYFDDWGQGILVTVSS

SEQ ID NO: 388 - protein, 126 aa, synthetic construct
QLQLQESGPGLLRPSETLSLTCTVSGGSTSRGSYYWGWIRQPPGKGLEWIGSIYYSGSTYSNPSLKSRVTMSLDTSKNQFSLKLSSVTAADTAVYYCARHSPYSSGWYIHFYFDSWGQGTLVTVSS

SEQ ID NO: 389 - protein, 129 aa, synthetic construct
EVQLVESGGGLVQPGGSLRLSCAASGFTFSSYWMHWVRQAPGKGLVWVSRINSDGSYTSYADSVKGRFTISRDNAKNTLYLQINSLRAEDTAVYYCARVRYYYDSSGSKGGYYYYMDVWGKGTTVTVSS

SEQ ID NO: 390 - protein, 117 aa, synthetic construct
QVQLVQSGAEVKKPGASVKVSCQSSGDVFTTYGFTWVRQAPGQGLEWMGWISTYKDDAEYSQRFQGRVTMTTDTSSSTAYMELRSLRSGDTAVYYCARGSKWELLHWGQGTPVTVSS

SEQ ID NO: 391 - protein, 121 aa, synthetic construct
QVTLKESGPALVKPTQTLTLTCTFSGFSLSTTGVRVSWIRQPPGKALEWLARIDWDDDKFYSTSLRTRLTISKDTSKNQVVLTMTNMDPVDTATYFCARLKYDNSGRGDYWGQGTLVTVSS

SEQ ID NO: 392 - protein, 122 aa, synthetic construct
EVQLVESGGGLVQPGGSLRLSCAASGFTFIDHWMHWVRQVPGKGLVWVCRISTDGSSTTYADFVKGRFAISRDNAKNTMSLQMRGLRAEDTAVYYCVRGSRFYFGSGSSDYLGQGTLVTVSS

SEQ ID NO: 393 - protein, 128 aa, synthetic construct
QVQLEESGGGLVKPGGSLRLSCAASGFTFSYYYMSWIRQAPGKGLEWVSYISSDGRIIYYADSVRGRFTISRNNAENSLSLLMNSLRTEDTAVYYCARAAPEYSNGWYLDGRGAFDIWGQGTMVTVSS

SEQ ID NO: 394 - protein, 123 aa, synthetic construct
EVQMVESGGGLVQPGGSLRLSCVASGFTFNNHWMHWVRQAPGKGLVWVSRISSDGISTSYADSVKGRFTISRDNAKNTLYMQMNSLRVDDTAVYFCARGKWSGYLLSHCFDQWGQGTLVTVSS

SEQ ID NO: 395 - protein, 124 aa, synthetic construct
QMQLVESGGGLVKPGGSLRLSCAASGFTFSDYYMSWIRQAPGKGLEWVSYINSGSTYTNYADSVKGRFTISRDNARNSLYLQMHSLRAEDAAVYYCARDYQWRELNDHWYFDLWGRGTLVTVSS

SEQ ID NO: 396 - protein, 119 aa, synthetic construct
EGQLVESGGGLVQPGGSLRLSCTASGFSVSSNYINWVRQVPGKGLEWVSIFYSSGGTDYTDAVKGRFTISRDNSKNTVHLQMNSLRVEDTAVYYCAGRAGSARGAFDVWGQGTMVSVSS

SEQ ID NO: 397 - protein, 118 aa, synthetic construct
EVQLVEFGGGLVKPGGSLRLSCAASGFTFSSYTMNWVRQAPGKGLEWVSSIGSSGSYMYYAGSVEGRFTISRDNANNSLYLQMNSVRVDDTAVYYCARIAGFEGVDHWGQGTLVTVSS

SEQ ID NO: 398 - protein, 124 aa, synthetic construct
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARLLYTKITLVRGALDYWGQGTLVTVSS

SEQ ID NO: 399 - protein, 117 aa, synthetic construct
QVQLVQSAAEVRKPGASVKVSCRPSGYRFTDYYLAWVRQAPGQGLEWMGYINPTAHGGTYGQKFQGRITLTADPSVSTVYMELTGLQADDTAVYYCVRSRSFYFDQWGQGTLVTVAT

SEQ ID NO: 400 - protein, 119 aa, synthetic construct
QVQLVQSGSELKKPGASVKVSCKASGYTFSNHAINWVRQAPGQGLEWMGWIKTNTGNPTYAQGFTGRFVFSLDTSVSTAYLQISSLKAEDTALYYCARGYISGYHFDYWGQGTLVTVSS

SEQ ID NO: 401 - protein, 119 aa, synthetic construct
QLHLQESGPGIVKPSETLSLTCIVSGDSIRTSSYYWGWIRQPPGKGLEWISNRYYRGSTNYSPSLKSRATLSVDTSKNQFSLRLTSVTAADTATYYCIREKSFFAEITWGQGTLVTVSS

SEQ ID NO: 402 - protein, 119 aa, synthetic construct
EVQLVESGGGLVQPGRSLRLSCAASGFTFDDNAMHWVRQAPGKGLEWVSGIGWNSGRIGYADSVKGRFTISRDNAKNSLYLQMNSLRAEDMALYFCAKEYGDYDAFDIWGQGTMVTVSS

SEQ ID NO: 403 - protein, 125 aa, synthetic construct
QVQMVESGGGVVLPGRSLRLSCAASGFTFRNYAMHWVRQAPGKGLEWVAVISYDGSNKYYADFVKGRFTISRDSSKNTLYLEMNSLRPEDTAVYYCAKDRVLGVLVPVAALFDYLGQGTLVTVSS

SEQ ID NO: 404 - protein, 122 aa, synthetic construct
QVQLVQSGAEVKKPGSSVKVSCKASGGTFSSYAISWVRQAPGQGFEWMGGIIPIFGTANYAQKFQGRVTITADESTSTAYMELSSLRSEDTAVYYCARDSDGDYVSGAFDIWGQGTMVTVSS

SEQ ID NO: 405 - protein, 126 aa, synthetic construct
EVQLVESGGGLVNPGGSLRLSCAASGFTFNSYSMNWVRQAPGKGLEWVSSIDTTSHYIYYAHSVRGRFTVSRDNAKNSLFLQVNSLRGEDTALYYCARAHCGGRGCFQRFDAFDIWGQGTMVTVSS

SEQ ID NO: 406 - protein, 118 aa, synthetic construct
EVQLVQSGADVKKPGESLRISCKGSGYSFPNYSIGWVRQMSGKGLEWMGNIDPADSETRYSPPFQGQVTMSVDKSISTAYLEWSSLKASDSGIYFCARLRAFVCPDFWGQGTLVTVSS

SEQ ID NO: 407 - protein, 118 aa, synthetic construct
QVQLQESGPGLVKPSETLSLTCTVSGGFISGYYWSWIRQPAGKGLEWIGHIYSSGIINDNPSLKSRVTMSVDTSKNQFYLNLTSVTAADTAVYYCARQGDYGDCFDNWGQGTLVTVSS

SEQ ID NO: 408 - protein, 120 aa, synthetic construct
EVQLVESGGGLVQPGGSLRLSCAASGFTFSSYEMNWVRQAPGKGLEWVSYISSSGSSIYYADSVKGRFTISRDNAKNSLYLQMNSLRAEDTAVYYCARDDLLWLYYFDYWGQGTLVTVSS

SEQ ID NO: 409 - protein, 125 aa, synthetic construct
EVQLVESGGGLVKPGGSLRLSCAASGFTFSTFGMNWVRQAPGKGLEWVSFISGSSNYIHSADSVKGRFTISRDNAKNSVYLQMNSLRAEDTAVYYCVRGGAWLGEFKHPRQFDYWGQGSLVTVSS

SEQ ID NO: 410 - protein, 121 aa, synthetic construct
EVQLVESGGSVVRPGGSLRLSCAASGFRFDDYGMSWVRQAPGKGLEWVSRINWNGGVTESADSVKGRFTISRDNTKNSLYLQMNSLRAEDTALYYCARQAVAGILWYFDLWGRGTLVTVSS

SEQ ID NO: 411 - protein, 128 aa, synthetic construct
DVQLLESGGGLVQPGGSLRLSCAVSGFTFKNYAMSWVRQAPGSGLVWVSSITTDGFSTYYADSVRGRFTISRDNSKNTLSLRMNSLGAEDTAVYFCARDHPRYSNGWYVSAMNYFDSWGRGIPVTVSS

SEQ ID NO: 412 - protein, 120 aa, synthetic construct
EVQLVESGGGLVQPGRSLRLSCAASGFTFDDYVMHWVRQAPGKGLEWVSGISWYNGSIGYADSVKGRFTISRDNAKNSLYLQMNSLRAEDTALYYCAKGGRGSYFHFDYWGQGTLVTVSS

SEQ ID NO: 413 - protein, 122 aa, synthetic construct
EVQLVESGGGLVQPGGSLRVSCAASGFTFSNYWMIWVRQPPGKGLVWVSRITSDGSSTAYADSVKGRFTISRDNAKNTLYLQMNSLRAEDTAVYYCARGSTSYFGSGSTNYWGQGTLVTVSS

SEQ ID NO: 414 - protein, 121 aa, synthetic construct
EAQLLESGGGLVQPGESLRLSCAASRFILSRHGLSWVRQAPGKGLEWVSAISGSGVTTFYADSVKGRFTVSRDNSINRLYLQMDSLRADDTAIYYCARDMSASGWAIFDSWGQGILVTVSS

SEQ ID NO: 415 - protein, 133 aa, synthetic construct
QEQLQQWGAGLLKPSETLSLTCAVYGGSFSNYYWTWIRQPPGKGLEWIGQIDHSGGTNYSPSLKSRVSILVDTSKSQFSLQLTSVTAADTAVYFCARGFIQRHFDYFLCSANRCPQSVFFDLWGRGTLVAVSS

SEQ ID NO: 416 - protein, 115 aa, synthetic construct
EAQLVESGGGLVQPGGSLTLSCAASRLGIDGEWMSWVRQAPGKGLEWVANIRWDRSQIFYADFVKGRFAISRDDAKKSLFLQMTGLRAEDTALYYCARKNSFDYWGRGTQVTVSS

SEQ ID NO: 417 - protein, 121 aa, synthetic construct
EVQVVESGGGLVKPGGSLRLSCTASELSFATYSLNWVRQAPGKGLEWVSSISSSGIRIYYGDPVKGRFTISRDNAKNSLFLQMNDLRVEDTAVYFCAIRKSCSGICPYDYWGQGTLVTVSS

SEQ ID NO: 418 - protein, 116 aa, synthetic construct
QVQLVESGGGVVQPGRSLRLSCVASGFSLRSFGMHWLRQAPGKGLEWVAVSWFDGSDQRYADAVKGRFTISRDNSKNTLYLQMDSLRAADTGVYHCGRSLYHNDNWGQGTLVTVSS

SEQ ID NO: 419 - protein, 124 aa, synthetic construct
QVQLVESGGGVVQPGRSLRLSCAASGFTFSSYGMHWVRQAPGKGLEWVAGIWYDGSKKYYGEAVKGRFTISRDDSTDTLYLQMNSLRVEDTAVYYCARDELYSSSPHSNWFDPWGQGTLVTVSS

SEQ ID NO: 420 - protein, 122 aa, synthetic construct
EVQLVESGGGVIPPGGSLRLSCAASGFIVNSNYMTWVRQAPGKGLEWVSVIYSSGNTYYADSVKGRFTISRDNSKNTLFLQMNSLRAEDTAVYYCSREPYWGGNQEDAFDIWGQGTMVTVSS

SEQ ID NO: 421 - protein, 118 aa, synthetic construct
EVQLVESGGGLVKPGGSLRLSCAASGFIFKNAWMNWVRQAPGKGLEWVGRIRRNSEGATTDYAAPLKGRFTILRDDSKNMLYLQMNSLKTEDTAVYYCTTGVDGHEHWGQGTLVTVSS

SEQ ID NO: 422 - protein, 121 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDLVVAATDGMDLWGQGTTVTVSS

SEQ ID NO: 423 - protein, 121 aa, synthetic construct
QVQLVESGGGLVKPGGSLRLSCAASGFTFSNAWMNWVRQAPGKGLEWVGRIKSKSVGGTTDYAAPVKGRFTISRDDSKNTLYLQMNSLKTEDTAVYYCTTETQLWFYFDYWGQGTLVTVSS

SEQ ID NO: 424 - protein, 121 aa, synthetic construct
QVQLVESGGGVVQPGRSLRLSCAASGFTFSRYGMHWVRQAPGKGLEWVALIWHDGTNGNYGDSVKGRFTISRDNSKSTVYLQMNSLRAEDTAVYYCAREDPNIVVAALDLWGQGTLVTVSS

SEQ ID NO: 425 - protein, 119 aa, synthetic construct
EVQLVQSGGGLVKPGGSLRLSCAASGFTFSIYSMNWVRQTPGKGLEWVSSISSSSSYIYYADSVKGRFTISRDNAKNSLYLQMNSLRAEDTAVYYCARGSYRFTPGDYWGQGTLVTVSS

SEQ ID NO: 426 - protein, 119 aa, synthetic construct
EVQLLESGGNLVQPGGSLRLSCAASGFTFTSYAMSWVRQAPGKGLEWVSVISGSGGSTYYVESVKGRFTISRDNSKSTVYLQMNSLRAEDTAVYYCARRGPVGYYFDYWGQGTLVTVSS

SEQ ID NO: 427 - protein, 120 aa, synthetic construct
EVQLVESGGGLVKPGGSLRLSCAASGFTFSSYSMSWVRQAPGKGLEWVSSISSSSTYIYYADSVKGRFTISRDNAKNSLYLQMNSLRAEDTAVYYCARDPVWSGFLFDYWGQGTLVTVSS

SEQ ID NO: 428 - protein, 120 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARTKLWTGSGMDVWGQGTTVTVSS

SEQ ID NO: 429 - protein, 120 aa, synthetic construct
QVQLVESGGGVVQPGRSLRLSCAASGFTFSIYAMHWVRQAPGKGLEWVAVIWYDGSNKYYADSVKGRFTISRDNSKNTLYLQMNSLRAEDTAVYYCASATGYSSGYPDYWGQGTLVTVSS

SEQ ID NO: 430 - protein, 123 aa, synthetic construct
QVQLVESGGGVVQPGRSLRLSCAASGFIFSSYGMHWVRQAPGKGLEWVAVVWYDGNNKYYADSVKGRFTISRDNSKNTLYLQMNSLRADDTAVYYCARDPYFLTGYHGYFDYWGQGTLVTVSS

SEQ ID NO: 431 - protein, 133 aa, synthetic construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARLRGDHCTNGVCPSPHYYYYYYMDVWGKGTTVTVSS

SEQ ID NO: 432 - protein, 120 aa, synthetic construct
QVQLVESGGGVVQPGGSLRLSCAASGFTLNSYGIQWVRQAPGKGLEWVAFIRYDGSKRYYADSVKGRFTISRDNSKNTLYLQMNSLRAEDTAVYYCAKDKRGQNYGMDVWGQGTTVTVSS

SEQ ID NO: 433 - protein, 117 aa, synthetic construct
QVQLQESGPGLVKPSETLSLTCSVSGGSVSSGSFYWSWIRQPPGKGLELIGYISYRGYTSYNPSLNSRATISVDTSKNSFSLKLNSVTAADTAVYYCARTGASPGYWGQGTLVTVSS

SEQ ID NO: 434 - protein, 113 aa, synthetic construct
EVQLVESGGALVQPGGSLRLSCAASGFTFSNYWMNWVRQAPGKGLEWVANIKPDGSQKYYVDSVKGRFTISRDNAKNSVYLQMNSLRVDDSAIYYCIPRDYKWGQGILVTVSS

SEQ ID NO: 435 - protein, 128 aa, synthetic construct
QLLLQQSGPGLVKPSETLSLTCSVSAGSNIRSSYYWGWIRQPPGKGLEWIGSTYWSGLAYYNPSLKRRVTISVDTSKNHFSLKLSSVTAADTAVYYCARQAYSSGWAAFGHYYFMDVWGKGTTVTVSS

SEQ ID NO: 436 - protein, 123 aa, synthetic construct
EVQLVDSGGGLVKPGGSLRLSCAASGFTFSTYTMNWVRQAPGKGLEWVSSITSSGNYIYYADSLKGRFTISRDNAKNSVYLQMNSLRGEDTAVYYCARVSGMAAAENYYMDVWGKGTTVTVSS

SEQ ID NO: 437 - protein, 125 aa, synthetic construct
QVQLVESGGGVVQPGRSLRLSCAASGFTFSNYGMHWVRQAPGKGLEWVALIWFDGSNKHYADSLQGRFTVSRDNSKNTLYLQMNSLRPDDTAIYYCAKSIGRVVISDLEVAFDYWGRGTLVTVSS

SEQ ID NO: 438 - protein, 129 aa, synthetic construct
DVQLLESGGGLVQPGGSLRLSCAASGFTFSRYAVNWVRQAPGKGLEWVSFISGPGVSTYYADSVKGRFTISRDNSKNTLYLQMNNLRAEDTAVYYCAKVGPFPSTYYDFWSDDYGLDVWGQGTTVTVSS

SEQ ID NO: 439 - protein, 122 aa, synthetic construct
EVQLLESGGGLVQPGGSLTLSCAGSGFTFKGYAMSWVRQAPGKGLEWVSAISGGGDKTYYADSVKGRITISRDNSKNTLYLRLDSLRAEDTANYYCAKSIQREVVIFRLHYWGQGTPVTVSS

SEQ ID NO: 440 - protein, 124 aa, synthetic construct
QVQLVQSGAEVKKPGSSVKVSCKASGGTSSTYAFSWVRQAPGQGLEWMGGIIPVFGTTNYAQKFQGRVTITAGESMTAAYMELSSLRSEDTAVYYCASRVVVVALNYYYGMDVWGQGTTVTVSS

SEQ ID NO: 441 - protein, 125 aa, synthetic construct
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARHAGYSYAGRGYNWFDPWGQGTLVTVSS

SEQ ID NO: 442 - protein, 120 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARVDLTEWLVLSYWGQGTLVTVSS

SEQ ID NO: 443 - protein, 125 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVSSDDFWSGYYQDFDYWGQGTLVTVSS

SEQ ID NO: 444 - protein, 122 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKAEDTAVYFCTTDLAQLWSNFDYWGQGTLVTVSS

SEQ ID NO: 445 - protein, 119 aa, synthetic construct
QVQLVQSGSELKKPGASVKLSCKASGYTFTTYAINWVRQAPGQGLEWMGWINTNTGNPMYAQGFTGRFVFSLDTSITTAYLQISSLKAEDTAVYSCARLGQTKNAFDIWGQGTMVTVSS

SEQ ID NO: 446 - protein, 118 aa, synthetic construct
ERQLEESGGGLVMPGRSLRLSCVASGFTVGDVYMSWVRQAPGKGLEWVGRIKSKTDGATTDYTPSLRGRFIITRDESKNTLDLQLNSLNIEDTGVYYCGSGGHYFDPWGQGTLVTVSS

SEQ ID NO: 447 - protein, 128 aa, synthetic construct
EEQLVEAGGGLVKPGGSLRLSCAASGFTFSYYSMNWVRQAPGKGLEWVSSVSPRSDHIYYADSVRGRFTISRDNAKNSLYLQMNSLRAEDTAIYYCVRDFRGEYDYLRGIYVYLPDYWGQGTLVTVSS

SEQ ID NO: 448 - protein, 121 aa, synthetic construct
EMQLVESGGGLVQPGGSLRLSCAASGFTFSTYWMSWVRQAPGKGLEWVANIQQDGIEQYFVDFVEGRFTISRDNAKNSLYLQMNSLRAEDTAVYFCARRGANSHNGAFDIWGQGTLVTVSS

SEQ ID NO: 449 - protein, 121 aa, synthetic construct
EVQLVQSGAEMKKPGESLKISCQGSGYKFTNYWIGWVRQMPGKGLEWMGIIFPGDSDTRYSPSFQGQVTISVDKSISTAYLQWSSLKASDTAIYYCARQGAHRNSSGFDYWGQGILVTVSS

SEQ ID NO: 450 - protein, 123 aa, synthetic construct
EVKLLTSGGGLVQPGGSLRLSCATSGFTFNRYALSWVRQAPGKGLEWVSGISATGAEVYYGDSVRGRFTVSRDNSKNTLYLQMKSLRVEDTAVYFCAKDAIAVSRNLYYFDYWGLGTLVTVSS

SEQ ID NO: 451 - protein, 123 aa, synthetic construct
EVQLVESGGGLVKPGGSLRLSCAASGFTFSNAWMSWVRQAPGKGLEWVGRIKSKTDGGTTDYTAPVKGRFTISRDDSKNTLYLQMNSLKTEDTAVYYCTTEPLEDWYPRFDPWGQGTLVTVSS

SEQ ID NO: 452 - protein, 125 aa, synthetic construct
EVQLVESGGGLVQPGGSLRLSCAASEVTFRGYWMHWVRQAPGKGLVWVSRIYTDGSSTNYADSVKGRFTISRDNAKNTLYLQMNSLRVEDTAVYYCAREANSGWYGVGQYYFDYWGQGTLVTVSS

SEQ ID NO: 453 - protein, 119 aa, synthetic construct
EVQLVESGGGLVQPGGSLRLSCTTSGFIFSTYWMTWVRQAPGKGLEWVATIQENGNEKYYVDSVKGRFTISRDNAKNSLYLQMNSLRAEDTALYYCVSVGEYRTASDYWGQGTLVTVSS

SEQ ID NO: 454 - protein, 125 aa, synthetic construct
QVQLQQWGAGLLKPSETLSLTCAVYGGSFSSYYWGWIRQPPGKGLEWIGDINHSGRTNYNTSFKGRVAVSVDPSKNQFTLKVTSVTAADTAVYFCASLEQYDSRWGWAADYFQHWGQGTLVTVSA

SEQ ID NO: 455 - protein, 120 aa, synthetic construct
EVQLVESGGVVVQPGGSLRLSCAASGFSFDDYTMHWVRHTPGKGLEWVSLITWDGVTTYYADSVKGRFTISRDNSKNSLYLQMNSLRTEDTALYYCAKEIKVAARTIDHWGQGTLVTVSS

SEQ ID NO: 456 - protein, 128 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAREGLLWFRDKPPLGLGGFDPWGQGTLVTVSS

SEQ ID NO: 457 - protein, 129 aa, synthetic construct
QVQLLDSGGGVVQPGRSLRLSCAASGFTFSSYAMHWVRQAPGKGLEWVAVISYDGNKKYYTDSVKGRLTISRDNSKNTVYLQMNSLRAEDTAVYYCARDLPVLRYFDWLPERHYGMDVWGQGTTVTVSS

SEQ ID NO: 458 - protein, 122 aa, synthetic construct
EVQLVESGGGLVQPGGSLRLSCEASGFTFSNYAMNWVRQAPGKGLEWISYIASGSTTKYYADSVMGRLTISRDNAKNTLFLELNSLRAEDTAVYYCARDQDGDYEVDAFDVWGQGTKVTVSS

SEQ ID NO: 459 - protein, 119 aa, synthetic construct
QVQLVESGGGVVQPGRSLRLSCAASGFTFSSLGMHWVRQAPGKGLEWVAVISFDGSNKYYADSVKGRFTISRDNSKNTLYLQMNSLRAEDTAVYYCAKLAGATLSFDYWGQGTLVTVSS

SEQ ID NO: 460 - protein, 125 aa, synthetic construct
QVHLVESGGGVVQPGRSLRLSCVASGFTLSSHAIHWVRQAPGKGLEWVAIISYDGSQKSYAGSVKGRFTVSRDTSKNTVSLQMNSLTVEDTAVYYCARDGFSGVMLFGMGYLDYWGQGTLVTVSS

SEQ ID NO: 461 - protein, 126 aa, synthetic construct
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCATSLYCIINKCSNYYGLDVWGQGTTVTVSS

SEQ ID NO: 462 - protein, 106 aa, synthetic construct
EIVMTQSPATLSVSPGERATLSCRASQSVSSNLAWYQQKPGQAPRLLIYGASARATGIPARFSGSGSGTEFTLTISSLQSEDFAVYYCQQYNNGRTFGQGTKVEIT

SEQ ID NO: 463 - protein, 111 aa, synthetic construct
DIVLTQTPLSSPVTLGQPASISCRSSQSLVYSDGNTYLNWLQQRPGQPPRLLIYKISNRFSGVPDRFSGSGAGTDFTLKISRVEAEDVGVYYCMQATQLYTFGQGTKLEIK

SEQ ID NO: 464 - protein, 106 aa, synthetic construct
DIQMTQSPSTLSASVGDRVTITCRASQSISSWLAWYQQKPGKAPKLLIYKASNLGSGVPSRFSGSGSGTEFTLTISSLQPDDFATYYCQQYNSYWTFGQGTKVEIK

SEQ ID NO: 465 - protein, 111 aa, synthetic construct
AVVMTQSPLSLPVTLGQPASISCRSSQSLVSSDGNTYLHWFQQRPGQSPRRLIYKVSNRDSGVPDRFSGSGSGTDFTLKISRVEAEDVGVYYCMQGTHWWTFGQGTKVEIK

SEQ ID NO: 466 - protein, 112 aa, synthetic construct
DIVMTQSPLSLPVTPGEPASISCRSSRSLLFRNGDTYLDWYLQKPGQSPQLLIYLASYRASGVPDRFSGSGSGTDFTLKISRVEAEDVGVYYCMQNLQTPATFGQGTKVEIK

SEQ ID NO: 467 - protein, 111 aa, synthetic construct
QSVLTQPPSVSGAPGQRVTISCTGNSFNIGAGYDVHWYQQLPGTAPKLLIYGNNNRPSGVPDRFSGSKSGTSASLAITGLQAEDEADYYCQSYDSSLSALVFGGGTKLTVL

SEQ ID NO: 468 - protein, 106 aa, synthetic construct
DIQMTQSPSTLSASVGDRVTITCRASQSISSWLAWYQQKPGKAPKLLIYKASSLESGVPSRFSGSDFGTEFTLTISSLQPDDFATYYCQQYNSYWTFGQGTKVEIK

SEQ ID NO: 469 - protein, 116 aa, synthetic construct
QPVLTQPPSASASLGASVTLTCTLSSGYSNYMVDWYQQRPGKGPRFVMRVGTGGIVGSKGDGIPDRFSVLGSGLNRYLTIKNIQEEDESDYYCGADYGSGSNFVWVFGGGTKLTVL

SEQ ID NO: 470 - protein, 106 aa, synthetic construct
DIQMTQSPSTLSTSVGDRVTITCRASQSINSWLAWYQQKPGKAPKLLIYKASTLEFGVPSRFSGSGSGTEFTLTISSLQPDDFATYYCQQYNRYWTFGQGTKVEIK

SEQ ID NO: 471 - protein, 107 aa, synthetic construct
DIQMTQSPSTLSASVGDRVTITCRASQSISRWLAWYQQHPGKAPKLLISGASSLENGVPSRFSGSGSGTDFALTISSLQPDDFATYYCQHYDSFFVTFGQGTKVELK

SEQ ID NO: 472 - protein, 108 aa, synthetic construct
EIVLTQSPGTLSLSPGERATLSCRASHPVTSTYLAWYQQKPGQAPRLLVYGASSRATGIPDRFSGSGSGTDFTLTVTRLEPEDFAVYFCQQYGGLPWTFGQGTKVEIK

SEQ ID NO: 473 - protein, 112 aa, synthetic construct
DVVMTQSPLSLPVTLGQPASISCRSGQSLLYSDGNTYLYWFQQRPGQSPRRLIYKVSNRDPGVPERFSGSGSGTDFTLKISRVEAEDVGIYFCLQGTHWPYTFGRGTKLEIK

SEQ ID NO: 474 - protein, 112 aa, synthetic construct
DILMTQTPLSLSVTPGQPASISCKSNESLLHGDGKTYFYWYLQKPGQSPHLLIYEVSNRFSGVPDRFSGSGSVTDFTLKISRVEAEDVGIYYCMQTIDLPWTFGQGTKVEIK

SEQ ID NO: 475 - protein, 106 aa, synthetic construct
DIQMTQSPSTLSASVGDRVTITCRASQSISSWLAWYQQKPGKAPKLLIYKASSLESGVPSRFSGSGSGTEFTLTINNLQPDDFATYYCQQYNSYWTFGQGTKVEIK

SEQ ID NO: 476 - protein, 106 aa, synthetic construct
DIQMTQSPSTLSASVGDRVTITCRASQSISRWLAWYQQKPGKAPKLLIYDASSLESGVPSRFSGSGSGTEFTLTISSLQPDDFATYYCQQYNSYWTFGQGTKVEIK

SEQ ID NO: 477 - protein, 111 aa, synthetic construct
DVVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGTHWYTFGQGTKLEIK

SEQ ID NO: 478 - protein, 107 aa, synthetic construct
NFMLTQPHSVSESPGKTVTISCTRSSGIIASNSVQWYQRRPGHSPTTVIYENDQRPSGVPDRFSGSIDSSSNSASLTISGLRTEDEADYYCQSYDWVFGGGTKLTVL

SEQ ID NO: 479 - protein, 106 aa, synthetic construct
EIVLTQSPATLSLSPGERATLSCRASHSVGSYLAWYQQKPGQAPRLLIYDTSSRAAGVPARFSGSGSGTDFTLTISSLEPEDFAVYYCQQRKNGFTFGPGTKVDIK

SEQ ID NO: 480 - protein, 112 aa, synthetic construct
QSMLTQPPSVSGAPGQRVTISCTGSSSNIGADYDVHWYQQLPGTAPKLLIYGNNNRPSGVPDRFSGSKSGTSASLAITGLQPEDEAEYYCQSYDSSLSGSFVFGTGTKVTVL

SEQ ID NO: 481 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSALVFGGGTKLTVL

SEQ ID NO: 482 - protein, 112 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGIYYCMQGTHWPRTFGGGTKVEIK

SEQ ID NO: 483 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWFTFGQGTRLEIK

SEQ ID NO: 484 - protein, 106 aa, synthetic construct
DIQMTQSPSTLSASVGDRVTITCRASQSIISSLAWYQQKPGKAPKLLIYKASSLENGVPSRFSGSGSGTEFTLTISSLQPDDFATYYCQQYNSYWTFGQGTKVEII

SEQ ID NO: 485 - protein, 106 aa, synthetic construct
YIQMTQSPSTLSASVGDRVTITCRASHNISSWLAWYQQKPGKAPNLLIYKASSLESEVPSRFSGSGSGTEFTLTISSLQPDDFATYYCQQYKNYWTFGQGTKVEIK

SEQ ID NO: 486 - protein, 112 aa, synthetic construct
DTVMTQTPLFLSVTPGQPASISCKSSQSLLHSDGRTYLYWYLQKPGQSPQLLIYEVSNRFSGVPERFSGSGSGTDFTLRISRVEAEDVGIYYCMQSIYLPITFGGGTKMEIK

SEQ ID NO: 487 - protein, 106 aa, synthetic construct
DIQMTQSPSTLSASVGDRVTITCRASQSISSWLAWYQQKPGKAPKLLIYRASSLESGVPPRFSGSGSGTEFTLTISSLQPDDFGIYYCQQYNSRWTFGQGTKVEIK

SEQ ID NO: 488 - protein, 106 aa, synthetic construct
DIQMTQSPSTLSASVGDRVTITCRPSQSISNWLAWYQQKPGKAPKLLIYKASSLESGVPSRFSGSGSGTEFTLTISGLQPDDFATYYCQQYNTYWTFGQGTKVEIK

SEQ ID NO: 489 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGFVFGTGTKVTVL

SEQ ID NO: 490 - protein, 111 aa, synthetic construct
DAVMTQTPLSLSVTPGQPASISCKSGQSLLHSDGKTYLYWYLQKPGQSPQLLIYEVSSRFSGVPDRFSGSGSGTDFTLKISRVEAEDVGVYYCMRGIHPRTFGQGTKVEIK

SEQ ID NO: 491 - protein, 106 aa, synthetic construct
EIVMTQSPATLSVSPGERATLSCRASQSVGSNLAWYQQKPGQAPRLLIYGASTRATGLPARFSGSGSATEFTLTISSLQSEDFAVYYCQQYNTWWTFGQGTKVEIK

SEQ ID NO: 492 - protein, 111 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHLYTFGQGTKLEIK

SEQ ID NO: 493 - protein, 106 aa, synthetic construct
DIQMTQSPSTLSASVGDRVTITCRASQSIGSWLAWYQQKPGKAPKLLIHEASSSESGVPSRFSGSGSGTEFTLTISSLQPDDFATYYCQQYNSYWTFGQGTKVEIK

SEQ ID NO: 494 - protein, 106 aa, synthetic construct
EIVMTQSPATLSVSPGERATLSCRASQSISSNLAWYQQKPGQAPRLLIYGASTRATGIPARFSGSGSGTEFTLTISSLQSEDFAVYYCQQYHDWWTFGQGTKVEIK

SEQ ID NO: 495 - protein, 106 aa, synthetic construct
DIQMTQSPSTLSASVGDRVTITCRASHSISNWLAWYQQKPGKAPRLLIYKASSLNSGVPSRFSGSASGTEFTLTISSLQPDDFATYFCQQYNAYWTFGQGTKVEIK

SEQ ID NO: 496 - protein, 106 aa, synthetic construct
EIVLTQSPATLSLSPGERATLSCRASQSISDYLAWYQQKPGQAPRLLIYDASFRATGIPARFSGSGSGTDFTLTISSLEPEDFAVYYCQQRSIGFTFGPGTKVDIK

SEQ ID NO: 497 - protein, 111 aa, synthetic construct
QFVLTQPPSVSGAPGQRVTISCTGSSFNIGAGYDVHWYQQLPEKAPKLLIYGSTNRASEVPDRFSGSKSGTSASLAITGLQPEDEADYYCQSYDSSLSGYVFGTGTKVTVL

SEQ ID NO: 498 - protein, 106 aa, synthetic construct
DIQMTQSPSTLSASVGDRVTITCRASQSISSWLAWYQQKPAEAPKLLIYEASTLESGVPSRFSGSGSGTEFTLTISSLQPDDFATYYCQQYNTYWTFGQGTKLEIK

SEQ ID NO: 499 - protein, 107 aa, synthetic construct
EIVLTQSPGTLSLSPGERATLSCRASQSVSGNYLAWYQRKPGQAFRLLIYGASSRATGIPDRFSGSGSGTDFTLTISRLEPEDFAVYFCQQYGRLWTFGQGTKVEIR

SEQ ID NO: 500 - protein, 106 aa, synthetic construct
DIQMTQSPSTLSASVGDRVTITCRASHSISSWLAWYQQKPGKAPKLLIYKASTLESGVPSRFSGSGSGTEFTLTISSLQPDDFATYYCQQYDYYWTFGQGTKVEIK

SEQ ID NO: 501 - protein, 107 aa, synthetic construct
EIVLTQSPATLSLSPGERATLSCRASQGVSSYLAWYQQKPGQAPRLLIYDASNRATGIPARFSGSGSGTDFTLTISSLEPEDFAVYYCQQRSNWPPTFGPGTKVDIK

SEQ ID NO: 502 - protein, 111 aa, synthetic construct
DIVMTQTPLSLSVSPGQPASISCKSSQSLLHSDGKTYLHWYMQKPGQSPQVLIYEVSSRFSGVPDRFSGSGSGTDFTLEISRVEAEDVGVYFCMQSIELWTFGQGTKVEIK

SEQ ID NO: 503 - protein, 106 aa, synthetic construct
EIVMTQSPATLSVSPGERATLSCRASQSVGSKLAWYQHKPGQAPRLLIYGASTRATGIPARFSGSGSGTEFTLTVSSLQSEDFAVYYCQQYNHWYTFGQGTKLEIK

SEQ ID NO: 504 - protein, 111 aa, synthetic construct
QSVLTQPPSVSGAPGQRVTISCTGSSSNIGAGYDVHWYQQLPGTAPKLLIYDNSNRPSGVPDRFSGSKSGTSASLAITGLQAEDEADYYCQSYDSSLSALVFGGGTKLTVL

SEQ ID NO: 505 - protein, 107 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQHNRYPPTFGGGTKVEIK

SEQ ID NO: 506 - protein, 111 aa, synthetic construct
QSVLTQPPSVSGAPGQRVTISCTGSSSNIGAGYDVHWYQQLPGTAPKLLIYGNTNRPSGVPDRFSGSKSGTSASLAIAGLQADDEADYYCQSYDSSLSVFVFGTGTKVTVL

SEQ ID NO: 507 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSYPPTFGQGTKVEIK

SEQ ID NO: 508 - protein, 114 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSILPLTFGGGTKVEIK

SEQ ID NO: 509 - protein, 112 aa, synthetic construct
DVVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGTHWPYTFGQGTKLEIK

SEQ ID NO: 510 - protein, 106 aa, synthetic construct
DIQMTQSPSSLSASVGDRVTITCQASQDISNYLNWYQQRPGKAPKVLIYDASNLAVGVPSRFSGSGSGTDFSFTISSLQPEDFATYFCQQYHTSFTFGPGTKVDIK

SEQ ID NO: 511 - protein, 111 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTHVLFGGGTKLTVL

SEQ ID NO: 512 - protein, 111 aa, synthetic construct
QSVLTQPPSVSGAPGQRVTIACSGSSSNIGAGYDVHWYQQLPGTAPKLLIYGNTNRPSGVPDRFSGSKSGTSASLAISGLQAEDEADYYCQSYDTSRGGLLFGGGTKLTVL

SEQ ID NO: 513 - protein, 116 aa, synthetic construct
QPVLTQPPSASASLGASVTITCTLSRGYNNYKVDWYQQRPGKGPRFVMRVGTGGIVGSKGDGIPDRFSVLGSGLNRYLTIKNIREEDEGDYHCGTDHASGNNFVYVFGTGTKVIVL

SEQ ID NO: 514 - protein, 106 aa, synthetic construct
DIQMTQSPSSLSASVGDRVTITCRASQDISNYLNWYQQKPGKAPKLLIYAASNLQSGVPSRFSGSGFGTEFSLTISNIQPEDVATYCCQQSHSLWTFGQGTKVEIK

SEQ ID NO: 515 - protein, 112 aa, synthetic construct
DIVMTQTPLSLSVTPGQPASISCSSSLSLLRSDGKTYLYWYLQKPGQPPQLLIYEVSNRFSGVPDRFSGSGSGTDFTLKISRVEAEDVGVYYCMQSVELPYTFGQGTKLEIK

SEQ ID NO: 516 - protein, 111 aa, synthetic construct
VVVMTQSPLSLPVTLGQPASISCKSSQSLVHSDGNTYLHWFQQRPGQSPRRLIYKVSNRDSGVPDRFSGSGSGTDFTLKISRVEAEDVGVYYCMQGTHWWTFGQGTKVEIK

SEQ ID NO: 517 - protein, 108 aa, synthetic construct
GVQMTQSPSTLSASVGDRVTLTCRASQSISSWLAWYQQKPGKAPKLLIYDASSLESGVPSRFSGSGSGTEFTLTISSLQPDDFATYYCQQYNSYSFWTFGQGTKVEIK

SEQ ID NO: 518 - protein, 111 aa, synthetic construct
DVVLTQSPLSLPVTLGQPASISCRSSQSLVHSDGNTYLNWFQQRPGQSPRRLIYKVSKRDYGVPERFSGSGSGTDFTLKISRVEAEDVGVYYCMQGTHWWTFGQGTKVEIK

SEQ ID NO: 519 - protein, 111 aa, synthetic construct
QSVLTQPPSVSGAPGQRVTISCTGGSSNIGAGYDIYWYQQLPGTAPKLLMYAYTNRPSGVPDRFSGSKSGTSASLAITGLQAEDEADYYCQSYDTSLDGWVFGGGTKLTVL

SEQ ID NO: 520 - protein, 112 aa, synthetic construct
QSVLTQPPSVSGAPGQRVTVSCTGSSSNIGAGYDVHWYQQLPGTAPKLLIYGNNNRPSGVPERFSGSRSGTSASLAITGLQADDEGDYYCQSYDRRLMNSWVFGGGTKLTVL

SEQ ID NO: 521 - protein, 106 aa, synthetic construct
EIVMTQSPATLYVSPGERATLSCRASQSVGRDLAWYQQKPGQAPRLLIYGASTRTTGVPARFSGSGSGTEFTLTLNSLQSEDFAVYYCQQYNKWYTFGQGTKLAIK

SEQ ID NO: 522 - protein, 106 aa, synthetic construct
EIVLTQSPATLSLSPGERATLSCRASQSVSSSVAWYQQKPGQAPRLLIYDASNRATDVPARFSGSGSGTDFTLTISSLEPEDFAVYYCQQRSSWFTFGQGTRLDIK

SEQ ID NO: 523 - protein, 112 aa, synthetic construct
DTVLTQTPLSLSVIPGQPASMSCKSSQSLLDNDGKTYLYWYLQKPGQSPQLLIYEVSNRFSGVPDRFSGSGSGTDFTLKISRVEAEDVGIYYCMQRIQLPWTFGQGTKVEIK

SEQ ID NO: 524 - protein, 111 aa, synthetic construct
NVVLTQTPLSSPVTLGQPASISCRSSQSLVYSDGNTYLNWLQQRPGQPPRLLIYKVSNRFTGVPDRFSGSGAGTDFTLNISRVEAEDVGVYFCMQATQLYTFGQGTKLEIK

SEQ ID NO: 525 - protein, 112 aa, synthetic construct
QSVLTQPPSVSGAPGQRVTISCTGSSSNIGADYDVHWYQQLPGTAPKLLIYGNNNRPSGVPDRFSGSKSGTSASLAITGLQAEDEADYYCQSYDSSLSSSWVFGGGTKVTVL

SEQ ID NO: 526 - protein, 106 aa, synthetic construct
DIQMTQSPPTLSASVGDRVTITCRASQSISTWLAWYQQKPGKAPKLLIYKASTLKSGVPSRFSGSGSGTEFTLTVSSLQPDDFATYYCQQYSYFWTFGQGTKVEIK

SEQ ID NO: 527 - protein, 108 aa, synthetic construct
GVQMTQSPSTLSASVGDRVTLTCRASQNINNWLAWYQQKPGKAPKLLIYEASILESGVPSRFSGSGSGTEFTLTISSLQPDDFATYYCQQYNDYSFWTFGQGTKVDIK

SEQ ID NO: 528 - protein, 111 aa, synthetic construct
QSVLTQPPSISGAPGQRVTISCTGAGSNIGAGYEVHWYQQLPGTAPKLLMFADNNRPSGVPDRFSGSKSGTSASLAITGLQAEDEADYYCQSYDISLKNFVFGTGTTVTVL

SEQ ID NO: 529 - protein, 108 aa, synthetic construct
DIQLTQSPSTLSASVGDRVTITCRASQSVNRWLAWYQQKPGKAPKLLIYEASTLEIGVPSRFSGSGSGTEFTLTISSLQPDDFATYHCQQYNSYSFWTFGQGTKVEIK

SEQ ID NO: 530 - protein, 106 aa, synthetic construct
EVVMTQSPATLSVSPGERATLSCRASQGVSSNLAWYQQKPGQPPRLLIYGASTRATGIPARFSGSGSGTEFTLTISSLQSEDFAVYYCQQYSNWWTFGQGTKVEIK

SEQ ID NO: 531 - protein, 111 aa, synthetic construct
DIVLTQTPLSSPVALGQPASISCRSSQSLVDSDGNTYLNWLQQRPGQPPRLLIYKISNRFSGVPDRFSGSGAGTDFTLKISRVEAEDVAVYYCMQATQLYTFGQGTKLEIK

SEQ ID NO: 532 - protein, 111 aa, synthetic construct
QSVLTQPPSVSGAPGQRVTISCTGSSSNIGAGYDVNWYQQFPGTAPKLLIYAYTNRPSGVPDRVSGSKSGTSASLAITGLQAEDEADYYCQTYDSSVDGWVFGGGTKLTVL

SEQ ID NO: 533 - protein, 106 aa, synthetic construct
EIVLTQSPATLSLSPGERATLSCRASQSVGRSLAWYQQKPGQSPRLLIYEASNRATGIPARFSGSGSGADFTLTISSLEPEDFAVYYCQQRSNWFTFGQGTRLEIK

SEQ ID NO: 534 - protein, 111 aa, synthetic construct
DIVLTQTPLLSPVTLGQPASISCRSSQSLVYSDGNTYLSWLQQRPGQPPRLLIYKISNRFSGVPDRFSGSGAGTSFTLKISRVEAEDVGVYYCMQATQLYTFGQGTKLEIK

SEQ ID NO: 535 - protein, 106 aa, synthetic construct
DIQMTQSPSTLSASVGDRVTITCRASQSISSWLAWYQQKPGKAPKLLIYRASILQTGVPSRFSGSGSGTEFTLTISSLQPDDFATYYCQQYDTYWTFGQGTKVEIK

SEQ ID NO: 536 - protein, 106 aa, synthetic construct
KIVMTQSPATLSVSPGERVTLSCRASQSISRKLAWYQQKPGLAPRLLIYGASTRATGIPARFSGSGSGTEFTLTISSLQSEDFALYYCQQYSNWYTFGQGTKLEIK

SEQ ID NO: 537 - protein, 111 aa, synthetic construct
QSVLTQPPSVSGAPGQRVTISCTGGSSNIGAGYDVHWYQQLPGTAPKLLIYGNTNRPSGVPDRFSGSKSDTSASLAITGLQAEDETDYYCQSYDSSLRRYVFGSGTKVTVL

SEQ ID NO: 538 - protein, 112 aa, synthetic construct
DIVMTQSPLSLPVTPGEPASISCRSSQSLLFSNGDNYLDWYLQKPGQSPQLLIYLGSYRASGVPDRFSGSGSGTDFTLKISRVEAEDVGVYFCMQSLQTPHTFGQGTKLEIK

SEQ ID NO: 539 - protein, 112 aa, synthetic construct
QSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSSSWVFGGGTKLTVL

SEQ ID NO: 540 - protein, 113 aa, synthetic construct
QSVLTQPPSVSGAPGQRVTISCTGGSSNIGAGYDVHWYQQLPGTAPKLLISHNNNRPSGVPDRFSGSKSGTSASLVIAGLQAEDEADYYCQSYDRRLSSSWVFGGGTRLTVLS

SEQ ID NO: 541 - protein, 111 aa, synthetic construct
HSVLTQPPSVSGAPGQRVTISCTGSTSNIGAGYDVYWYQQLPGTAPKLLIYGNNNRPSGVPDRFSGSTSGTSASLAITGLQAEDEADYYCQSYDTTLTGWEFGGGTKLTVL

SEQ ID NO: 542 - protein, 106 aa, synthetic construct
EIVLTQSPATLSLSPGEKATLSCRASQSVSTSLAWYQQKPGQAPRLLIYEASNRATGVPARFSGSGSGTDFTLTISSLEPEDFAVYYCQQRSSWFTFGQGTRLEIK

SEQ ID NO: 543 - protein, 106 aa, synthetic construct
EIVLTQSPATLSLSPGERATLSCRASQSISSYLAWYQQKPGQAPRLLIYDASTRSTGIPTRFSGSGSGTDFTLTISSLEPEDFAIYFCQQRSSGFTFGPGTTVDVK

SEQ ID NO: 544 - protein, 111 aa, synthetic construct; identical to SEQ ID NO: 477

SEQ ID NO: 545 - protein, 106 aa, synthetic construct
EIVLTQSPATLSLSPGERATLSCRASQSVSSYLAWYQQKPGQAPRLLIYDASKRATDIPARFSGSGSGTDFTLTISSLEPEDFAVYYCQQRSVGFTFGPGTKVDIK

SEQ ID NO: 546 - protein, 106 aa, synthetic construct
DIQMTQSPSTLSASVGDRVTITCRASQSISRWLAWYQQKPGKAPKLLIYKASSLQSGVPSRFSGSGSGTEFTLTISSLQPDDFATYYCQQYNSYWTFGQGTKVEIK

SEQ ID NO: 547 - protein, 106 aa, synthetic construct
DIQMTQSPSTLSASVGDRVTITCRASQSVSRWLAWYQQKPGKAPKLLIYDASSLESGVPSRFSGSGSGTEFTLTISSLQPDDFATYYCQQYNRYWTFGQGTKVEIK

SEQ ID NO: 548 - protein, 111 aa, synthetic construct
QSVLTQPPSVSGAPGQRVTIPCTGSSSNIGAGYDVYWYQQLPGTAPKLLIYGDTNRPSGVPDRFSGSKSGTSASLAITGLQAEDEADYYCQSYDSTVSGWEFGGGTKLTVL

SEQ ID NO: 549 - protein, 112 aa, synthetic construct
DIVMTQTPLSLSVTPGQPASISCKSSQSLLDNDGKTYLYWYLQKPGQSPQLLIYEVSNRFSGVPDRFSGSGSGTDFTLKISRVEAEDVGVYYCMQRIDLPWTFGQGTKVEIK

SEQ ID NO: 550 - protein, 112 aa, synthetic construct
QSVLTQPPSVSGAPGQTVTISCTGSSSNIGANYDVHWYQQRPGTAPKLLIYGNMNRPSGVPDRFSASKSGTSASLAITGLQADDEADYYCQSYDRRLMASWVFGGGTKLTVL

SEQ ID NO: 551 - protein, 111 aa, synthetic construct
QSVLTQPPSVSGAPGQRVTISCTGSSFNIGAGYDVHWYQQIPGTAPKVLIFGYTNRPSGVPDRFSGSKSGTSASLAITGLQAEDEADYYCQSYDSSLSGYVFGTGTKVTVL

SEQ ID NO: 552 - protein, 112 aa, synthetic construct
DIVLTQTPLSLSVTPGQPASMSCKSSQSLLYSDVKTYLHWYLQKPGQPPQLLMYEVSNRFSGVPDRFSGSGSGTDFTLKISRVEAEDVGVYYCMQTIDLPWTFGQGTKVEIK

SEQ ID NO: 553 - protein, 112 aa, synthetic construct
VITMTQTPLSLSVTPGQPASISCKSSQSLLHSDGKTYLYWYLQKPGQSPQLLMYEVSKRVSGAPDRFSGSGSGTDFTLKISRVESEDVGVYYCMQSIEIPLTFGGGTRVEIK

SEQ ID NO: 554 - protein, 112 aa, synthetic construct
DIVMTQTPLSLSVTPGQPASISCKSSQSLLDNDGKTYLYWYLQKPGQSPQLVIYEVSNRFSGVPDRFSGSGSGTDFTLKIRRVEAEDVGVYYCMQRIDLPWTFGQGTKVEIK

SEQ ID NO: 555 - protein, 111 aa, synthetic construct
QSVLTQPPSVSGAPGQRVTISCTGSSSNIGAGYDVYWYQQLPGTAPKLLIYGNDNRPSGVPDRFSGSKSGTSASLAITGLQAGDEADYYCQSYDTIVSGWEFGGGTKLTVL

SEQ ID NO: 556 - protein, 112 aa, synthetic construct
DIVMTQSPLSLPVTPGEPASISCRSSQSLLFSNGDNYLDWYLQKPGQSPQLLIYLGSNRAPGVPDRFSGSGSGTDFTLKISRVEAEDVGVYYCMQALQTPHTFGQGTKLEIR

SEQ ID NO: 557 - protein, 112 aa, synthetic construct
DVVMTQSPLSLPVTLGQPASISCRSSQSLLYSDGNTYLFWFQQRPGQSPRRLIYKVSNRDSGVPDRFSGSGSGTDFTLKISRVEAEDVGVYFCLQGTHWPYTFGPGTKLEIK

SEQ ID NO: 558 - protein, 111 aa, synthetic construct
QSVLTQPPSVSGAPGQRVTISCTGSSSNIGAGYDVYWYQQFPGTAPKLLIYGDSHRPSGVPDRFSGSKSGASASLAITGLQAADEADYYCQSYDTSLSGWEFGGGTKVTVL

SEQ ID NO: 559 - protein, 111 aa, synthetic construct
QSGVTQPPSVSGAPGQRVTISCTGSSSNIGAGYDVYWYQQLPGTAPKLLIYGNTNRPSGVPDRFSGSKSGTSASLAITGLQAEDEADYYCQSYDTSLSGWVFGGGTKLTVL

SEQ ID NO: 560 - protein, 112 aa, synthetic construct
DIVMTQTPLSLSVTPGQPASISCKSTESLLHRDGKTYLYWYLQKPGQPPQLLIYEVSNRFSGVPDRFSGSGSVTDFTLKISRVEAEDIGVYYCMQTIDLPWTFGQGTKVEIK

SEQ ID NO: 561 - protein, 112 aa, synthetic construct
DILMTQTPLSLSVTPGQPASISCKSSQSLLDNDGKTYLYWYLQKPGQSPQLLIYEVSNRFSGVPDRFSGSGSGTDFTLRIRRVEAEDVGVYYCFQRIDLPWTFGQGTKVEIK

SEQ ID NO: 562 - protein, 111 aa, synthetic construct
QSVLTQPPSVSGAPGQRVTVSCTGSGSNIGANYEVHWYQQLPGTAPKLLIYGDNNRPSGLPDRFSASKSGTSASLAITGLQAEDEADYYCQSYDSSLSGYVFGSGTKVTVL

SEQ ID NO: 563 - protein, 112 aa, synthetic construct
EIVMTQTPLSLSVTPGQPASISCKSSQSLLHSDGKTYLYWYLQKPGQSPQLLIYEVSKRFSGVPDRFSGSGSGTDFTLQISRVEAEDVGVYYCLQTIEIPLTFGGGTKVEIK

SEQ ID NO: 564 - protein, 109 aa, synthetic construct
SFVLTQPPSVSVAPGQTASITCGGNNIGSKSVHWYQQKPGQAPVLVVYDDSVRPSGIPERFSGSNSGNTATLTITRVEAGDEADYSCQVWDTSSDRLYVFGTGTRVTVL

SEQ ID NO: 565 - protein, 108 aa, synthetic construct
DIQMTQSPSTLSASVGDRVTITCRASQSISTWLAWYQQRPGKAPKLLIYKASSLETGVPSRFSGSGSGTEFTLTISSLQPDDFATYYCHQYSSYSPRSFGQGTKLEIK

SEQ ID NO: 566 - protein, 107 aa, synthetic construct
DIQMTQSPSSLSASVGNRVTITCRASQIVGSSLHWYQQKPGKAPKLLIHHASSLQSGVPSRFSGSGSGTDFTLTIGSLQPEDFATYYCQQSYSTPYTFGQGTNLEIK

SEQ ID NO: 567 - protein, 108 aa, synthetic construct
PYLLTQPPSVSVAPGTTARITCGENNIGSQSVHWYQQKPGQAPVLVVYDDRVRPSGIPERFSGSNSGNTATLTISRVEAGDEADYYCHVWYSSAHHQVFGGGTKLTVL

SEQ ID NO: 568 - protein, 107 aa, synthetic construct
SYELTQPPSLSVSPGQTATITCSGDGLGDNYASWYQQKPGQSPVLVIYQDNKRPSGIPERFSGSNSGNTATLTISGTQAMDEADYYCQTWDSSTLWVFGGGTKLTVL

SEQ ID NO: 569 - protein, 110 aa, synthetic construct
QSVLTQPPSVSGAPGQRVTISCTGSSSNIGADYDVHWYQHLPGTAPKLLIYGNTNRPSGVPDRFSGSKSGTSASLAITGLQAEDEADYYCQSYDSSLYVVFGGGTKLTVL

SEQ ID NO: 570 - protein, 110 aa, synthetic construct
QSVLTQPPSVSGAPGQRVTISCTWSSSNIGAALDVHWYQQVPGTAPKLLIYSNTNRPSGVPDRFSGSKSATSASLAITGLQAEDEADYYCQSYDSSLSVLFGGGTKLTVL

SEQ ID NO: 571 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQSYTFGQGTKLEIK

SEQ ID NO: 572 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGLVFGGGTKLAVL

SEQ ID NO: 573 - protein, 111 aa, synthetic construct
DIVMTQTPLSLSVTPGQPASISCKSSQSLLHSDGKTYLYWYLQKPGQAPQLLIYEASKRFAGVPDRFRGSGSGTDFTLKISRVEAEDVGVYYCMQSIQLWTFGQGTKVEIK

SEQ ID NO: 574 - protein, 106 aa, synthetic construct
DIQMTQSPSTLSASVGDRVTITCRASQSISRWLAWYQQKPGKAPKLLIYKASNLESGVPSRFSGSGSGTEFTLTISSLQPDDFATYYCQQYKTLWTFGQGTKVEIK

SEQ ID NO: 575 - protein, 106 aa, synthetic construct
DIQMTQSPSTLSASVGDRVTITCRASQSISTWLAWYQQKPGKAPKLLIYKASTLESGVPSRFSGSGSGTEFTLTISSLQPDDFATYYCQQYNFYWTFGQGTKVEVK

SEQ ID NO: 576 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTWTFGQGTKVEIK

SEQ ID NO: 577 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYFTFGQGTKLEIK

SEQ ID NO: 578 - protein, 112 aa, synthetic construct
QSVLTQPPSVSGAPGQRVTISCTGSSSNIGAGYDVHWYQQLPGTAPKLLIYSNSNRPSGVPDRFSGSKSGTSASLAITGLQAEDEADYYCQSYDSSLSGLYVFGTGTKVTVL

SEQ ID NO: 579 - protein, 111 aa, synthetic construct
QSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSDYVFGTGTRVTVL

SEQ ID NO: 580 - protein, 112 aa, synthetic construct
DIVLTQSPVSLPVTPGEPASIACRSSQSLLYSNGDYYLDWYLQKPGQSPQLLIYLGSNRASGVPDRFSGSGSGTDFTLKISGVEAEDVGIYYCMQSLQTPLTFGGGTKVEIK

SEQ ID NO: 581 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAIYYCQQRSNWWTFGQGTKVEIK

SEQ ID NO: 582 - protein, 106 aa, synthetic construct
EIVMTQSPVTLSVSPGERATLSCRASQSVSSDLAWYQQKPGQAPRLLIYGASTRATGIPARFSGSGSGTEFTLTISSLQSEDFAVYYCHQYNNWYTFGQGTKLEIK

SEQ ID NO: 583 - protein, 112 aa, synthetic construct
DIVMTQSPLSLPVTPGEPASISCRSSLSLLHSNGYNYLDWYLQKPGQSPQLLIYLGSNRASGVPDRFSGSGSGTDFTLKISRVEAEDVGVYYCMQALETPITFGQGTRLEIK

SEQ ID NO: 584 - protein, 116 aa, synthetic construct
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGINFVWVFGGGTKLTVL

SEQ ID NO: 585 - protein, 113 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHRPLFTFGPGTKVDIK

SEQ ID NO: 586 - protein, 106 aa, synthetic construct
DIQMTQSPSTLSASVGDRVTITCRASQNIGTWLAWYQQRPGKAPKLLIYKASSLQTGVPSRFSGSGSGTEFTLTISSLQPDDFASYYCQQYNFYWTFGQGTKVEIK

SEQ ID NO: 587 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGLLFGGGTKLTVL

SEQ ID NO: 588 - protein, 111 aa, synthetic construct
DVVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGTHWWTFGQGTKVEIK

SEQ ID NO: 589 - protein, 106 aa, synthetic construct
EIVMRQSPATLSVSPGERATLSCRASQSVSSNLAWYQLKPGQAPRLLIFGASTRATGIPARFSGSGSGTEFTLTISNLQSEDFAVYYCQQYNDWWTFGQGTKVEIK

SEQ ID NO: 590 - protein, 106 aa, synthetic construct
DIQMTQSPSTLSASVGDRVTITCRASHSIYTWLAWYQQKPGKAPKLLIYKASTLESGVPSRFSGSGSGTEFTLTISSLQPDDFATYYCQQYNRYWTFGQGTKVEIK

SEQ ID NO: 591 - protein, 106 aa, synthetic construct
QSALTQPASVSGSPGQSITISCTGTSSDVQSYNYVSWYQQHPGKAPKLIIFEVSNRPSGVSNRFSGSKSGNTASLNISGLHGEDEADYYCSSKRVFGGGTKLTVLS

SEQ ID NO: 592 - protein, 106 aa, synthetic construct
EIVLTQSPATLSLSPGERATLSCRASHSVSSYLAWYQQKPGQAPRLLIYDASNRATGIPARFSGSGSETDFTLTISSLEPEDFAVYYCQQRNSWWTFGQGTKVEIK

SEQ ID NO: 593 - protein, 112 aa, synthetic construct
EIVMTQTPLSLSVTPGQPASITCKSSQSLLNTDGKTYLYWYLQKPGQPPQLLIYEVSNRFSGVPDRFSGSGSGTDFTLKISRVEAEDVGVYYCMQSIQLPWTFGQGTKVEIK

SEQ ID NO: 594 - protein, 106 aa, synthetic construct
DIQMTQSPSTLSASVGDRVTITCRASQSISSWLAWYQQKPGNAPKLLIYKASSLESGVPSRFSGSGSGTEFTLTISSLQPDDFATYYCQQYNSYWTFGQGTKVEIK

SEQ ID NO: 595 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSAPWTFGQGTKVEIK

SEQ ID NO: 596 - protein, 113 aa, synthetic construct
DIVMTQSPDSLAVSLGEGATINCKSSQSVLYTSNNKNYLAWYQQKPGQPPKLLIYWASTRESGVPDRFSGSGSGTDFTLTISSLQAEDVAVYYCQQYYSTPLTFGGGTKVEIK

SEQ ID NO: 597 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSSFTFGPGTKVDIK

SEQ ID NO: 598 - protein, 112 aa, synthetic construct
DVVMTQSPLSLPVTLGQPASISCRSSQSLVHNDGNTYLNWFQQRPGQSPRRLIHKVSNRDSGVPDRFSGSGSGTDFTLKISRVEAEDVGVYYCMQGTHWPYTFGQGTKLEIK

SEQ ID NO: 599 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYWTFGQGTKVEIK

SEQ ID NO: 600 - protein, 111 aa, synthetic construct
DIVMTQTPLSSPVTLGQPASFSCRSSQSLVHSDGNTYLSWLQQRPGQPPRLLIYKISNRFSGVPDRFSGSGAGTDFTLKISRVEAEDVGVYYCMQATQWFTFGQGTKLEIK

SEQ ID NO: 601 - protein, 106 aa, synthetic construct
KIVMTQSPATLSVSPGEGATLSCRASQSVSSNLAWYQQKPGQAPRLLIYGASTRATGIPARFSGSGSGTEFTLTISSLQSEDFAVYYCQQYNHWFTFGPGTKVDIK

SEQ ID NO: 602 - protein, 112 aa, synthetic construct
DIVLTQSPLSLPVTPGESASISCKSSQSLLHSNGYNYLAWYLQKPGQSPQLLIYLGSHRASGVPDRFSGSGSGTDFTLKISRVEAEDVGVYYCMQPLQTPLTFGGGTKVEIK

SEQ ID NO: 603 - protein, 106 aa, synthetic construct
EIVMTQSPATLSVSPGERATLSCRASQSVSSNLAWYQQKPGQAPRLLIYGASTRATGIPATFSGSGSGTEFTLTISSLQSEDFAVYYCQQYNNWWTFGQGTKVEIK

SEQ ID NO: 604 - protein, 111 aa, synthetic construct
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLWTFGQGTKVEIK

SEQ ID NO: 605 - protein, 108 aa, synthetic construct
DIQMTQSPSTLSASVGDRVTITCRASQSISSWVAWYQQKPGKAPKFLIYKASILQSGVPSRFSGSGSGTEFTLTISSLQPDDFATYYCQQYNSYSGLTFGGGTKVEIK

SEQ ID NO: 606 - protein, 106 aa, synthetic construct
EIVLTQSPATLSLSPGERATLSCRASQNVNTYLAWYQQKPGRAPRLLIYDASFRATGIPARFSGSGSGTDFTLTISSLEPEDFALYYCQQRSSGFTFGPGTKVDIK

SEQ ID NO: 607 - protein, 106 aa, synthetic construct
DIVLTQSPATLSLSPGERATLSCRASQSVGSYLGWYQQKPGQAPRLLIYDASNRATGIPARFSGSGSGTDFTLTISSLEPEDFAIYYCQQRSSGFTFGPGTKVDIK

SEQ ID NO: 608 - protein, 106 aa, synthetic construct
EIVLTQSPATLSLSPGERATLSCRASQSISSYLAWYQQKRGQAPRLLIYDASNRATGIPARFSGSGSGTDFTLTISSLEPEDFAVYYCQQRSSGFTFGPGTKVDIK

SEQ ID NO: 609 - protein, 106 aa, synthetic construct
EIVLTQSPATLSLSPGERATLSCRASQSVSNYLGWYQQKPGQAPRLLIYDASVRATGIPARFSGSGSGTDFTLTINSLEPEDFAVYYCQQRSIGFTFGPGTKVDVR

SEQ ID NO: 610 - protein, 106 aa, synthetic construct
EIVLTQSPATLSLSPGERATLSCRASQSVTTYLAWYQQKPGQAPRLLIYDASNRATGIPDRFSGSGSGTDFTLSINSLEPEDFAVYYCQQRSKGFTFGPGTKVDFK

SEQ ID NO: 611 - protein, 106 aa, synthetic construct
EIVLTQSPATLSLSPGERATLSCRASQSVSSYLGWYQQKPGQAPRLLIYDASNRATGIPARFSGSGSGTDFTLTISSLEPEDFAVYYCQQRSSGFTFGPGTKVDFK

SEQ ID NO: 612 - protein, 108 aa, synthetic construct
GVQMTQSPSTLSASVGDRVTLTCRASQSVSSWLAWYQQKPGKAPKLLIYDASRLESGVPSRFSGSGSGTEFTLTISSLQPDDFATYYCQQYASYSFWTFGQGTKVEIK

SEQ ID NO: 613 - protein, 108 aa, synthetic construct
DIQMTQSPSTLSASVGDRVTITCRASQSISSWLAWYQQKPGKAPKLLIFDASSLENGVPSRFSGSGSGTEFTLTISSLQPDDFATYFCQQYNSYSFWTFGQGTKVDIK

SEQ ID NO: 614 - protein, 108 aa, synthetic construct
DIQMTQSPSTLSASVGDRVTITCRASQSINNWLAWYQLKPGKAPKLLIYDASNLQTGVPSRFSGSGSGTEFTLTISSLQPDDFATYYCKQYNSYSFWTFGQGTKVEIK

SEQ ID NO: 615 - protein, 106 aa, synthetic construct
EIVLTQSPATLSLSPGERATLSCRASQSVSSYLAWYQQKPGQAPRLLIYGATNRATGIPARFSGSGSGTDFTLTINRLEPEDFAVYYCQQRSSWFTFGQGTRLEIK

SEQ ID NO: 616 - protein, 106 aa, synthetic construct
EIVLTQSPATLSLSPGERAILSCRASQSVSTDLAWYQQKPGQAPRLLIYGASNRVTGIPARFSGSGSGTDFTLTVSSLEPEDFAIYYCQQRSNGFTFGQGTRLEIK

SEQ ID NO: 617 - protein, 106 aa, synthetic construct
EIVLTQSPATLSVSPGERATLSCRASQSVSSSLAWYQQKPGQAPRLLIYDASNRATGIPARFSGSGSGTDFTLTISTLEPEDFAVYYCQQRSNGFTFGQGTRLEIK

SEQ ID NO: 618 - protein, 106 aa, synthetic construct
EIVLTQSPATLSLSPGEGATLSCRASQSVGNYLAWYQQKPGQAPRLLIYDASSRATGIPARFSGSGSGTDFTLTISSLEPEDFAVYYCQQRSNWFTFGQGTRLEIK

SEQ ID NO: 619 - protein, 106 aa, synthetic construct
EIVLTQSPATLSLSPGERATLSCRASQSVRTNLAWYQQKPGQAPRLLIYDASNRATGIPARFSGSGSGTDFTLTISSLEPEDFAVYYCQQRSNWFTFGQGTRLEIK

SEQ ID NO: 620 - protein, 106 aa, synthetic construct
EIVLTQSPATLSLSPGERATLSCRASQSVSTHLAWYQQKPGQAPRLLIYDASNRATGIPARFSGSGSGTDFTLTISSLEPEDFAIYYCQQRSQWFTFGQGTRLEIK

SEQ ID NO: 621 - protein, 106 aa, synthetic construct
EIVLTQSPATLSLSPGERATLSCRASQSVSSSLAWYQQKPGQAPRLLIYEASNRATGIPPRFSGSGSGTDFTLTISSLEPEDFAVYYCQQRRNGFTFGQGTRLEMK

SEQ ID NO: 622 - protein, 106 aa, synthetic construct
QIVLTQSPATLSLSPGERATLSCRASQSVSSSLAWYQQKPGQAPRLLIYDASNRATGIPARFSGSGSGTDFTLTISSLEPEDFALYYCQQRSKGFTFGQGTRLEIK

SEQ ID NO: 623 - protein, 106 aa, synthetic construct
EIVLTQSPATLSLSPGERATLSCRASQSVGSSLAWYQQKPGQAPRLLMYDTSNRASGIPARFSGSGSGTDFTLTISSLEPEDFAIYYCQQRANWFTFGQGTRLEIK

SEQ ID NO: 624 - protein, 106 aa, synthetic construct
EIVLTQSPATLSLSPGERATLSCRASQSVSSYLGWYQQKPGQAPRLLIYDASKRATGIPARFSGSGSGTDFTLTISSLEPEDFAVYYCQQRSNWFTFGQGTRLEIK

SEQ ID NO: 625 - protein, 106 aa, synthetic construct
EIVLTQSPATLSLSPGERATLSCRASQSVTTYVAWYQQKPGQAPRLLIYGASNRATGIPARFSGSGSGTDFTLTISSLEPEDFAVYYCQQRSQWFTFGQGTRLEIK

SEQ ID NO: 626 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGPGTKVDIK

SEQ ID NO: 627 - protein, 112 aa, synthetic construct
DIVMTQTPLSLSVTPGQPASISCKSSESLLNTDGKTYFYWYLQKPGQSPQLLIYEVSNRFSGVPDRFSGSGSGTDFTLKISRVEAEDVGIYYCMQTIQLPWTFGQGTKVEIK

SEQ ID NO: 628 - protein, 112 aa, synthetic construct
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLPWTFGQGTKVEIK

SEQ ID NO: 629 - protein, 111 aa, synthetic construct
DIVMTQTPLSLSVTPGQPASISCKSSQSLLHSDGKTYLYWYLQKPGQSPHLLIYEVSSRFSGVPDRFSGSGSGTDFTLKISRVEAEDVGVYYCMQGIHPRTFGQGTKVEIK

SEQ ID NO: 630 - protein, 111 aa, synthetic construct
DVVLTQTPLSSPVTLGQPASISCRSSQSLVYSDGNTYLSWLQQRPGQPPRLLIYKIFNRFSGVPDRFSGSGAGTDFTLNISRVEAEDVGVYYCMQATQLYTFGQGTKLEIK

SEQ ID NO: 631 - protein, 111 aa, synthetic construct
DIVMTQTPLSSPVTLGQPASISCRSSHTLVSSDGNTYLNWLQQRPGQPPRLLIYKISNRFSGVPDRFSGSGAGTDFTLKISRVEAEDVGVYYCMQATQLYTFGQGTKLEIK

SEQ ID NO: 632 - protein, 112 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYTFGQGTKLEIK

SEQ ID NO: 633 - protein, 112 aa, synthetic construct
DVVMTQSPLSLPVTLGQPASISCRSGQSLVYSDGNTYLYWFQQRPGQSPRRLIYKVSNRDSGVPDRFSGSGSGTDFTLKISRVEAEDVGVYFCLQGTHWPYTFGQGTKLEIK

SEQ ID NO: 634 - protein, 112 aa, synthetic construct
DVVMTQSPLSLPVTLGQPASISCRSSQSLLYTDGNTYLYWFQQRPGQSPRRLIYKVSNRDSGVPDRFSGSGSGTDFTLKISRVEAEDVGVYYCMQGTHWPYTFGQGTKLEIK

SEQ ID NO: 635 - protein, 106 aa, synthetic construct
EIVMTQSPATLSVSPGERATLSCRASQSISSNLAWYQQKPGQAPRLLIYGASTRATGIPARFSGSGSGTEFTLTISSLQSEDFAVYFCQQYNNWYTFGQGTKLEIK

SEQ ID NO: 636 - protein, 106 aa, synthetic construct
EIVMTQSPATLSLSPGDRATLSCRASQSVSSNLAWYQQKRGQAPRLLIYGASTKATGVPARFSGSGSGTEFTLTISSLQSEDFAVYYCQQYKNWYTFGQGTKLEIR

SEQ ID NO: 637 - protein, 106 aa, synthetic construct
EIVMTQSPATLSVSPGERATLSCRASQSVNSKLAWYQQKPGQAPRLLIYGASTRATDIPGRFSGSGSGTEFTLTISSLQSEDFAVYYCQQYSNWYTFGQGTKLEIK

SEQ ID NO: 638 - protein, 106 aa, synthetic construct
EIVMTQSPATLSVSPGERATLSCRASQSVSSKLAWYQQKPGQAPRLLIYGASNRATGIPARFSGSGSGTEFTLTISSLQSEDFAVYYCQQYKHYYTFGQGTKLEIK

SEQ ID NO: 639 - protein, 106 aa, synthetic construct
EIVMTQTPATLSVSPGERATLSCRASQSVSSNLAWYQQKPGQAPRLLIYGASTRATGIPARFSGSGSGTEFTLTISSLESEDFAIYYCQQYQNWYTFGQGTMLEIK

SEQ ID NO: 640 - protein, 106 aa, synthetic construct
QIVMTQSPATLSVSPGERATLSCRASQSVSSNLAWYQQKRGQAPRLIIFGASTRATGIPARFSGSGSGTEFTLTISNLQSEDFAVYYCQQYNNWYTFGQGTKLEIR

SEQ ID NO: 641 - protein, 106 aa, synthetic construct
EIVMTQSPATLSVSPGERATLSCRASRSVGSKLAWYQQKRGQAPRLLIYGASTRATGIPARFSGSGSGTEFTLTISSLQSADFALYYCQQYNQWYTFGQGTKLEIR

SEQ ID NO: 642 - protein, 106 aa, synthetic construct
EILLQQSPATLSVSPGERVTLSCRASQSVSNNLAWYQQKPGQAPRLLIYGASTRATGIPPRFSGSGSGTEFTLTISSLQSEDFAIYYCQQYNRWYTFGQGTRLEIK

SEQ ID NO: 643 - protein, 112 aa, synthetic construct
VVVMTQTPLSLSVTPGQPASISCKSSQSLLHSDGRTYLYWYLQKPGQSPQLLMYEVSNRFSGVPDRFSGSGSGTDFALIISRVEAEDVGVYYCMQSIELPITFGGGTKVEIK

SEQ ID NO: 644 - protein, 112 aa, synthetic construct
DNVMTQTPLSLSVTPGQPASISCKSSQSLLHSNGKTYLYWYLQKPGQSPQLLIYEVSNRFSGVPDRFSGSGSGTDFTLKISRVEAEDVGVYYCMQSIHLPLTFGGGTKVEIK

SEQ ID NO: 645 - protein, 112 aa, synthetic construct
DIVVTQSPLSLPVTPGEPASISCRSSQSLLFWNGDNYLDWYLQKPGQSPQLVIYLGSYRAPGVPDRFSGSGSGTDFTLKISRVEAEDVGVYYCMQSLQTPHTFGQGTKLEIR

SEQ ID NO: 646 - protein, 112 aa, synthetic construct
DIVMTQSPLSLPVTPGEPASISCRSSQSLLYSNGDDYLDWYLQKPGQSPQLLIYLGSNRASGVPDRFSGSGSGTDFTLKISRVEAEDVGVYYCMQTLQTPHTFGQGTKLEIK

SEQ ID NO: 647 - protein, 112 aa, synthetic construct
DIVMTQSPLSLPVTPGEPASISCRSSQSLLFYNGDNYLDWYLQKPGQSPQLLIYLGSNRASGVPDRFSGSGSGTDFTLKISRVEAEDVGVYYCMQSIQTPHTFGQGTKLEIK

SEQ ID NO: 648 - protein, 112 aa, synthetic construct
DIVMTQSPLSLPVTPGEPASISCRSSQSLLYSNGDNYLDWYLQKPGQSPQLLIYLGSNRAPGVPDRFSGSGSGTDFKLKISRVEAEDVGVYYCMQALQTPHTFGQGTKLEIK

SEQ ID NO: 649 - protein, 112 aa, synthetic construct
DVVMTQSPRSLPVTPGEPASISCRSSQTLLFYNGDNYLDWYLQKPGQSPQLLIYLGSYRAPGVPDRFSGSGSGTDFTLKISRVEAEDVGVYYCMQSIQTPHTFGQGTKLEIK

SEQ ID NO: 650 - protein, 112 aa, synthetic construct
DIVMTQSPLSLPVTPGEPASISCRSSQSLLYSNGDHYLDWYLQKPGQSPQVLIYLGSNRAPGVPDRFSGSGSGTDFTLKISRVEAEDVGVYYCMQALQTPHTFGQGTKLEIK

SEQ ID NO: 651 - protein, 112 aa, synthetic construct
DIVMTQSPLSLPVTPGEPASISCRSSQSLLFRNGDNYLDWYLQKPGQSPQLLIYLGSNRASGVPGRFSASGSGTDFTLKISRVEAEDVGVYFCMQTLQTPHTFGQGTKLQIK

SEQ ID NO: 652 - protein, 112 aa, synthetic construct
DIVMTQSPLSLPVTPGEPASISCRSSQSLLFRNGDNYLDWYLQKPGQSPQLLIYLGSNRASGVPDRFSGSGSGTDFTLRISRVEAEDVGIYYCMQSLQTPHTFGQGTKLDIK

SEQ ID NO: 653 - protein, 112 aa, synthetic construct
DIVMTQSPLSLPVTPGEPASISCRSSQSLLFWNGDNYLDWYLQKPGQSPQLLIYLGSNRASGAPDRFSGSGSGTDFTLKISRVEAEDVGVYYCMQSLQTPHTFGQGTKLEIK

SEQ ID NO: 654 - protein, 106 aa, synthetic construct
DIQMTQSPSTLSASVGDRVTLTCRASQTINNWVAWYQQKPGKAPKLLIYKASSQKSGVPSRFSGSGFGTEFTLTISSLQPDDFATYYCQQYSLYWTFGQGTKVDIK

SEQ ID NO: 655 - protein, 106 aa, synthetic construct
DIQMTQSPSTLSASVGDRVTITCRASQSISVWLAWYQQKPGEAPKLLIYKASSLQPGVPSRFSGSGSGTDFTLTISNLQADDFATYYCQQYNYFWTFGQGTKVEIK

SEQ ID NO: 656 - protein, 106 aa, synthetic construct
DIQLTQSPSTLSASVGDRVTITCRASHDIISWLAWYQQKPGKAPKLLIYKASSLKSGVPSRFSGSGSGTEFTLTISSLQPDDFATYYCQQYNTFWTFGQGTKVEVK

SEQ ID NO: 657 - protein, 106 aa, synthetic construct
DIQMTQSPSTLSASVGDRVTITCRASQSISGWLAWYQQKPGKAPKLLIYRASSLKSGVPSRFSGSGFGTEFTLTISSLQPDDFATYYCQQYNFYWTFGQGTKVDVK

SEQ ID NO: 658 - protein, 111 aa, synthetic construct
QSVLTQAPSVSGAPGQRVTISCTGSSSNIGAGYDVHWYQQLPGTAPKLLIYGNNNRPSGVPDRFSGSKSGTSASLAITGLQAEDEADYYCQSYDSSLSGYVFGTGTKVTVL

SEQ ID NO: 659 - protein, 111 aa, synthetic construct
QFVLTQPPSVSGTPGQRVTISCTGGSFNTGAGYEVHWYQQLPGTAPRLLIYGNNNRPSGVPDRFSGSRSGTSASLAITGLQAEDETDYYCQSYDSSLSGYVFGTGTKVTVL

SEQ ID NO: 660 - protein, 111 aa, synthetic construct
QSVLTQPPSVSGAPGQRVTISCSGSGSNIGANYDVHWYQQFPGTAPQLLIFGDSNRPSGVPDRFSGSKSGTSASLAITGLQAEDEADYYCQSYDSRLSRYVFGTGTKVTVL

SEQ ID NO: 661 - protein, 111 aa, synthetic construct
QSMLTQPPSVSGAPGQRVTISCSGSGSNIGANYDVHWYQQFPGTAPQLLIFGTINRPSGVPDRFSGSRSGTSASLAITGLRAEDEADYYCQSYDSRLSRYVFGTGTKVTVL

SEQ ID NO: 662 - protein, 112 aa, synthetic construct
QSVLTQPPSVSGAPGQRVTISCTGSSSNIGAGYDVHWYQQLPGTAPKLLIYGNINRPSGVPDRFSGSKSGTSASLAITGLQAEDEADYYCQSYDRRLSGSWVFGGGTKLTVL

SEQ ID NO: 663 - protein, 112 aa, synthetic construct
QSVLTQPPSVSGAPGQRVTISCTGSSSNIGAGYDVHWYQQLPGTAPKLLIYGNNNRPSGVPDRFSGSKSGTSASLAITGLQAEDEADYYCQSYDSSLSASWVFGGGTRLTVL

SEQ ID NO: 664 - protein, 112 aa, synthetic construct
QSVLTQPPSVSGAPGQTVTISCTGSSSNIGAGHDVHWYQHLPGTAPKIIIYGDSNRPSGVPDRFSGSKTGTSASLAITGLQAEDEADYYCQSFDRRLSASWVFGGGTKLTVL

SEQ ID NO: 665 - protein, 106 aa, synthetic construct
EIVMTQSPATLSVSPGERATLSCRASQSVSSNLAWYQQKPGQAPRLLIYGAFTRATGIPARFSGSGSGTEFTLTISSLQSEDFAVYYCQQYKRWWTFGQGTKVEIK

SEQ ID NO: 666 - protein, 106 aa, synthetic construct
YIQMTQSPSTLSASVGDRVTITCRASQSISSWLAWYQQKPGKAPKLLIYKASTLQSGVPSRFSGSGSGTEFTLTISSLQPDDFATYYCQQYHSFWTFGQGTKVDIK

SEQ ID NO: 667 - protein, 106 aa, synthetic construct
DIQMTQSPSTLSASVGDRVTITCRASQSISSWLAWYQQKPGKAPKLLIYKASRLESGVPSRFSGSGSGTEFTLTISSLLPDDFATYYCQQYESYWTFGQGTKVEVK

SEQ ID NO: 668 - protein, 106 aa, synthetic construct
YIQMTQSPSTLSASLGDRVTITCRASHSISTWLAWYQQKPGKAPKLLIYRTSTLESGVPSRFSGSGSGTEFTLTISSLQPDDFATYYCQQYSSYWTFGQGTKVEIK

SEQ ID NO: 669 - protein, 112 aa, synthetic construct
QSMLTQPPSVSGAPGQRVTISCTGSSSNIGAGYDVHWYQQLPGTAPKLLIYGNNNRPSGVPDRFSGSKSGTSASLAITGLQAEDEADYYCQSYDISLSTSFVFGTGTKVTVL

SEQ ID NO: 670 - protein, 106 aa, synthetic construct
DIQMTQSPSSLSASVGERVIITCQASQDINHYLNWYQQKPGTAPKLLIYDASKLETGVPSRFSGSGSGTDFTFTIASLQPEDFATYYCQHYDLYPLFGGGTKVDVK

SEQ ID NO: 671 - protein, 107 aa, synthetic construct
DIQMTQSPSSLSASVGDRVTITCQASQDISNYLIWYQQKPGKAPKLLIYDASNLETGVPSRFSGSGSGTHFTFTISSLQPEDIATYYCQQFDNLPLTFGGGTKVEIK

SEQ ID NO: 672 - protein, 110 aa, synthetic construct
QSVLTQPPSVSAAPGQKVTISCSGSSSNIGYDYVSWYRQLPGTAPKLLIYDNDKRPSGIPDRFSGSKSGTSATLGITGLQTGDEAVYYCGTWDSSLSAGVFGGGTKLTVL

SEQ ID NO: 673 - protein, 106 aa, synthetic construct
SYVLTQPPSVSVAPGETATITCGGNNIGSKSVHWYQQRPGQAPVLVIYYDSNRPSGIPERLSGSNSGNTATLTITWVEAGDEADYYCQVWDSYTAVFGGGTKLTVL

SEQ ID NO: 674 - protein, 106 aa, synthetic construct
SYSLTQPPSVSVSPGQTASITCSGDKLGDKYVSWYQQKPGQSPVLVIYKDNKRPSVIPERFSGSNSGNTATLSISGTQALDEADYYCQAWDSSAVVFGGGTKLTVL

SEQ ID NO: 675 - protein, 107 aa, synthetic construct
EIVLTQSPGTLSLSPGERATLSCRASQSLNSNYLAWYQQKPGQAPRLLIYGASSRATGIPDRFSGSGSGTDFTLTISRLEPEDFAVYYCQQYGNSHTFGQGTKVEIK

SEQ ID NO: 676 - protein, 107 aa, synthetic construct
DIQMTQSPSSLSASIGDRVTITCRASQTISSSLSWYQQRPGKAPRLLIYSASSLQRGVPSRFSGSGSGTDFTLTITRLQPEDFATYYCQQSYSTPFTFGPGTTVDMK

SEQ ID NO: 677 - protein, 109 aa, synthetic construct
SYELTQPPSVSLSPGQTARITCSADVLSKQYAYWYQQKPGQAPVLVMSKDTERPSGIPERFSGSRSGTTVTLTISGVQAEDEADYYCQSMDSDPAYVLIFGGGTKLTVL

SEQ ID NO: 678 - protein, 106 aa, synthetic construct; identical to SEQ ID NO: 599

SEQ ID NO: 679 - protein, 106 aa, synthetic construct
EIVMTQSPATLSVSPGERATLSCRASQRVNSDLAWYQQKPGQPPRLLIYGASTRATGIPARFSGSGSGTEFTLTISSLQSEDFAVYYCQQYNNWFTFGLGTKLEIK

SEQ ID NO: 680 - protein, 110 aa, synthetic construct
SYVLTQAPSVSVAPGKTATITCGGNNIGSKSVYWYQQRPGQAPVLVVFDDSDRPSGIPERFSGSNSGNTATLSISRVEAGDEADYYCQVWDTTTDHPGVFGGGTKLTVLS

SEQ ID NO: 681 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGQGTRLEIK

SEQ ID NO: 682 - protein, 106 aa, synthetic construct
DIQMTQSPSTLSASVGDRVTITCRASQSISSWLAWYQQKPGKAPKLLIFKTSSLESGVPSRFSGSGSGTEFTLTISSLQPDDFATYYCQQYNSLFTFGPGTKVDIK

SEQ ID NO: 683 - protein, 108 aa, synthetic construct
EIVLTQSPGTLSLSPGERATLSCRASQSVSSNYLAWYQQKPGQAPRLLIYGASTRATGIPDRFSGSGSGTDFTLTISRLEPEDFAVYYCQQYGSSPYTFGQGTKLDIK

SEQ ID NO: 684 - protein, 111 aa, synthetic construct
QSVLTQPPSVSGAPGQRVTISCTGSSSNIGAGYDVAWYQQLPGTAPKLLIYSNNNRPSGVPDRFSGSKSGTSVSLAITGLQAEDEADYYCQSYDSSLTIFVFGTGTKVTVL

SEQ ID NO: 685 - protein, 112 aa, synthetic construct
DVVLTQSPLSLPVTLGQPASISCRSSHSLVYTDGNTYLNWFQQRPGQSPRRLIYKVSNRDFGVPDRFSGSGSGTDFTLKISRVEAEDVGVYYCMQGAHWPPTFGGGTKVEIK

SEQ ID NO: 686 - protein, 106 aa, synthetic construct
DIPMTQSPSTLSASVGDRVTITCRASQNINSWLAWYQQKPGKAPKLLIYKASSLESGVPTRFSGSGSGTEFTLTISSLQPDDFATYYCQQYNFYWSFGQGTKVEIK

SEQ ID NO: 687 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYSPYTFGQGTKLEIK

SEQ ID NO: 688 - protein, 106 aa, synthetic construct
QSALTQPASVSGSPGQSITISCTVTSNYDLVSWYQQHPGKAPKLIIYAVSNRPSGISHRFSGSKSGNTASLTISGLQAEDEADYYCSSYLISNARVFGTGTEVTVL

SEQ ID NO: 689 - protein, 107 aa, synthetic construct
EIVLTQSPATLSLSPGERATLSCRASQSVSSRYLAWYQQKPGQAPRLLIYGASNRATGIPDRFSGSGSGTDFTLTISRLEPEDFAVYYCQQYGSGITFGQGTRLEIK

SEQ ID NO: 690 - protein, 107 aa, synthetic construct
DIQMTQSPSSLSASVGDRVTITCRASQTINSYLNWYQQQPGRAPKLLIYAASRLQSGVPSRFSGSGSGTDFTLTITNLQVDDFATYYCQQSYSPPFTFGQGAKLDIK

SEQ ID NO: 691 - protein, 107 aa, synthetic construct
DIQMTQSPSSLSASVGDRVTITCQASQDISNYLNWYQQKPGKAPKLLIYDASNLETGVPSRFSGSGSGTDFTFTINSLQPEDIATYYCQQYDNLPPTFGQGTKLEIK

SEQ ID NO: 692 - protein, 108 aa, synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQYGRSTRTFGQGTKVEIK

SEQ ID NO: 693 - protein, 110 aa, synthetic construct
QAGLTQPPSVSRGLRQTATLTCTGNSDNVGYEGAAWLQQHQGHPPKLLSYRNNNRPSGISARFSASWSGNTASLTITGLQPEDEADYYCSAWDRSLTGWLFGGGTKLTVL

SEQ ID NO: 694 - protein, 110 aa, synthetic construct
QAGLTQPPSVSKGLRQTATLTCTGNSNNVGNEGAAWLQQHQGHPPKLLFYRNNNRPSGISERFSASRSGNTASLTITGLQPEDEADYYCSAWDNSLSVWVLGGGTKVTVL

SEQ ID NO: 695 - protein, 115 aa, synthetic construct
QAVLTQPSSLSASPGASASLACTLRSDINVRTNMVYWYRQKPGSPPQYLLRYKTDSDKQQGSGVPSRFSGSRDDSANAGILLISGLQSEDEGDYFCMIYYARAYVFGAGTRLTVL

SEQ ID NO: 696 - protein, 113 aa, synthetic construct
DIVMTQSPDSLAVSLGERAIIRCKSSQTLLYTYNNKDYITWYQQKPGQPPRLLIYWASTRESGVPDRFSGSGSGTDFTLTISSLQAEDVATYYCQQFSRTPYTFGQGTKLEIR

SEQ ID NO: 697 - protein, 110 aa, synthetic construct
EAGLTQPPSVSRALRQTATLTCSGNGNNVGNEGAAWMQQHLGHPPKLLSDRSHNRPSGISERFSASRSGNTASLTISGLQPEDEADYYCTAWDNSINGWIFGGGTKLTVL

SEQ ID NO: 698 - protein, 113 aa, synthetic construct
DIVMTQSPDSLALSLGERATINCRSSQSVLYSSNRKHNLAWYQQRPGHPPTLLIYWASTRQSGVPDRFSGRGSGTNFTLTISALQAEDVGVYYCQQYSIVPWTFGQGTKVEIK

SEQ ID NO: 699 - protein, 115 aa, synthetic construct
QAVLTQPSSLSASPGASASLTCTLRSGINVGAYRIYWYHQKPGSPPQYLLRYKSDSDKHQGSGVPRRFSGSKDASANAGILLISGLQSEDDGDYYCLIWHNSAWVFGGGTKLTVL

SEQ ID NO: 700 - protein, 111 aa, synthetic construct
QSVLTQPPSVSGAPGQRVTISCTGSSSNIGAGYDVHWYQQLPRTAPKLLIYGNNNRPSGVPDRFSGSKSGTSASLAVTGLQPEDEANYYCQSYDSSLSGSVFGGGTKLTVL

SEQ ID NO: 701 - protein, 110 aa, synthetic construct
QTVVTQEPSFSVSPGETVTLTCGLTSGSVSTSSYASWYQQAPGQAPRTLIVTTNTRSSGVPDRFSGSILGDKAALTITGARADDESDYYCLLYVGGGSWVFGGGTKLTVL

SEQ ID NO: 702 - protein, 113 aa, synthetic construct
DIVMTQTPLSLSVAPGEPASISCRSSQSLLDSDDGNTYLDWYLQRPGQSPHRLIYTLSYRASGVPDRFSGNGSGTDFTLKISGFEADDVGVYFCMQRIKFPLTFGGGTKVGIK

SEQ ID NO: 703 - protein, 108 aa, synthetic construct
EILLTQSPGTLSLSPGERATLSCRASETISSDYLAWYLQRPGQPPRLLIFGASSRATDIPDRFSGSGSGTDFTLTISRLEPEDFAVYYCQQYGTTPYTFGQGTKLEIK

SEQ ID NO: 704 - protein, 112 aa, synthetic construct
QSVLTQPPSVSGAPGQRVTISCTGSSSNIGAGYNVHWYQHLPGKAPKLLISENSNRPSGVPDRFSGSKSGTSASLVITGVQAEDEGDYYCQSYGSSLTAFYVFGTGTKVTVL

SEQ ID NO: 705 - protein, 112 aa, synthetic construct
QSALTQPPSVSGAPGQRVTISCTGTSSNIGAHYDVHWYQVVPGAAPKLLIYDNSNRPSGVPDRFSGSKSGSSASLAIAGLQPEDEADYYCQSYENSLSGFYVFGTGTKVTVL

SEQ ID NO: 706 - protein, 111 aa, synthetic construct
QSVLTQPPSVSGAPGQRVTISCTGSSSNIGAGFYVHWYQQLPGTAPKLLIYADTNRPSGVPARFSGSKSGTSAFLAITGLQAEDEADYYCQSHDSSFDGGVFGGGTRLTVL

SEQ ID NO: 707 - protein, 107 aa, synthetic construct
DIQLTQSPSFVSASVGDRVTITCRASQGIGNYLAWYQQRPGKAPRLLIYAASTLQSGVPSRFGGSGSGTEFTLTINSLQPEDFASYYCQQPSGYVWTFGQGTKVEIK

SEQ ID NO: 708 - protein, 111 aa, synthetic construct
QSVLTQPPSVSGAPGQSVTISCTGDSSNIGASFDVHWYQQLPGTAPKLLIYGNNNRASGVPDRFFGSKSGTSASLAITGLQAEDEADYYCQAYDSSPRAWVFGGGTTLTVL

SEQ ID NO: 709 - protein, 112 aa, synthetic construct
DIVLTQAPLSSPVTLGQPASVSCRSSQSLVHSDGNTYLSWLQQRPGQPPRLLIYKISNRFSGVPDKFSGSGAGTDFTLKISRVEAEDVGVYYCMQTTQFPLTFGGGTRVEIK

SEQ ID NO: 710 - protein, 107 aa, synthetic construct
DIQMTQSPSTLSASVGDRVTITCRASQSVSTWLAWYQQKPGQAPNLLIYKASTLKPGVPSRFSGSGSGTEFTLTISSLQPDDSATYHCQQYSNDVYTFGQGTRLEIK

SEQ ID NO: 711 - protein, 108 aa, synthetic construct
QSVLTQPPSVSGAPGQRVTISCSGISSSYDVHWYQQLPGTAPKLLIYGNSNRPSGVPDRFSGSKSGTSASLAITGLQAEDEADYYCQSYGSSLNAFYVFGTGTKVSVL

SEQ ID NO: 712 - protein, 112 aa, synthetic construct
DIVMTQSPLSLPVTPGESASISCRSSRSLLYSDGHNYVDWYLQKPGQSPQLLIYLGSNRASGVPDRFSGSGSGTDFTLKISRVEAEDVGVYYCMQALQTPLTFGGGTKVEIK

SEQ ID NO: 713 - protein, 111 aa, synthetic construct
NFMLTQPPSVSESPGKTVTISCIRSSGSIASNYVQWYQQRPGRAPTTVIYENNRRPSGVPDRFSGSIDSSSNSASLTISGLQTEDEAAYYCQSYVPRIQAVLGGGTLLTVL

SEQ ID NO: 714 - protein, 112 aa, synthetic construct
DIVMTQSPLSLSVTPGEAASISCRSSQSLLNSNGNTYLDWYLQKPGQSPQLLISLASNRASGVPDRFSGSGSGTDFILRIWRVEPEDVGVYYCMQALQAPFSFGQGTKLEVK

SEQ ID NO: 715 - protein, 108 aa, synthetic construct
SYELTQPPSVSVSPGHTARITCSGDALPKQYAYWYQQKSGQAPVLVIYEDSKRPPGIPERFSGSSSGATVTLTISGVQAEDEADYYCQSADSSTSYVVFGGGTKLTVL

SEQ ID NO: 716 - protein, 108 aa, synthetic construct
EVVMTQSPATLSVSPGGRATLSCRASPGINNNLAWYQLKPGQAPRLLIYDASTRASGVPARFSGSGSGTEFTLTITSLQSEDFALYFCQQYNNWPPYTFGQGTTLDIK

SEQ ID NO: 717 - protein, 109 aa, synthetic construct
SSKLTQDPAVTVALGQTVRITCQGDSLNDNFATWFQQKPGQAPVLVIFGQNSRPSGIPGRFSASNSGNTASLTVTGAQAEDEAVYYCNSRDNSEYVWLFGGGTKLTVLS

SEQ ID NO: 718 - protein, 108 aa, synthetic construct
EIVLTQSPGTLSLSPGERATLSCRASQYVSSTYVAWYQHKPGQSPRLLISGASSRATGIPDRFSGSASGTDFTLTISRVEPEDFAVYYCQQYGSSPYNFGPGTKVEIK

SEQ ID NO: 719 - protein, 111 aa, synthetic construct
QSALTQPRSVSGSPGQSVTISCTGTSSDVGAYNYVSWYQQFPGKVPKLIIYDVGARPSGVPDRFSASKSGNTASLTISGLQTDDEADYYCCSFAHNYNVVFGGGTKVTVLS

SEQ ID NO: 720 - protein, 112 aa, synthetic construct
QSALTQPASVSGSPGQSITISCTGTSSDVGGHDLVSWYQQHPGRAPKLIIFEVTKRPSRISNRFSGSKSGNTASLTISGLRAEDEADYFCCSYAGPFISGVVFGGGTKLTVL

SEQ ID NO: 721 - protein, 107 aa, synthetic construct
DIQLTQSPSFLSASIGDRVTITCRASQDLSSFLAWYQQKPGKAPKFLIHAASTLQSGVPSRFSGRKSGTEFTLTISSLQPEDYATYYCLQLNNYFTTFGGGTKVEIK

SEQ ID NO: 722 - protein, 108 aa, synthetic construct
EVVLTQSPATLSLSPGESATLSCRASQSVGSYLAWYQQKPGQAPRLLIYAASKRATGIPARFSGSGSGTDFTLTVSRLEPEDFAVYYCQQRSSWPPYTFGQGTKLEIK

SEQ ID NO: 723 - protein, 110 aa, synthetic construct
QSALTQPPSASGSPGQSVTISCTGPSSAVANYNYVSWYQQLPGKAPKLILYEVNQRPSGVPDRFSGAKSGNTASLTVSGLQAEDEGDYYCSSYGGTNNWVFGGGTKLTVL

SEQ ID NO: 724 - protein, 111 aa, synthetic construct
QSALTQPRSVSGSPRQSVTLSCTGTSSDVGGYNYVSWYQQHPGKAPKLMIYDVSKRPSGVPARFSGSKSGNTASLTISGLQAEDEADYYCCSYSGSYTPVVFGGGTKLTVL

SEQ ID NO: 725 - protein, 108 aa, synthetic construct
EIVLTQSPATLSLSPGARATLSCRASQSISNYLAWYQQKPGQAPRLLIYDTYTRATGIPARFSGSGSGTDFTLTISSLEPEDFAVYYCQQRSSWPLLTFGGGTRVEIK

SEQ ID NO: 726 - protein, 106 aa, synthetic construct
EIVMTQSPATLSVSPGERATLSCRASQSIGSYLAWYQQKPGQAPRLLIFGASTRATGIPARFSGSGSGTEFTLTISSLQSEEFAVYYCQQYSNGWTFGPGTKVEVK

SEQ ID NO: 727 - protein, 110 aa, synthetic construct
QSVLTQPPSASGTPGQRVTISCSGSSSNIGSNHVYWYQQVPGTAPKPLIYLNSQRPSGVPDRFSGSKSGTSASLAISGLRSEDEADYYCAAWDDSLSGYVFGSGTKLTVL

SEQ ID NO: 728 - protein, 110 aa, synthetic construct
QSALTQPPSASGSPGQSVTISCTGTSSDIGAYNFVSWYQQHTGKAPKLIVYEVIKRPSGVPDRFSGSKSGNTASLTVSGLQTEDEADYYCSSYAGNKNVMFGGGTRLTVL

SEQ ID NO: 729 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSLWTFGQGTKVEIK

SEQ ID NO: 730 - protein, 116 aa, synthetic construct
QPVMTQPPSASASPGASVTLTCTLGSDYSNYKVDWYQQRPGTGPRFLMRVGPRGIVGSKGDGIPNRFSVLASGLNRFLTIENLQEEDEAVYHCAADQGNGDTLRWVFGGGTKLTVL

SEQ ID NO: 731 - protein, 115 aa, synthetic construct
QAVLTQPSSLSASPGASASLTCTLRSGINVGIYRIYWYQQKPGSPPQYLLRYKSDSDKQQGSGVPSRFSGSKDASANAGILLISGLQSEDEADYYCMVWHSNAVVFGGGTKLTVL

SEQ ID NO: 732 - protein, 115 aa, synthetic construct
QAVLTQPSSLSASPGASASLTCTLHSGIDVGLYRVYWYQQKPGSPPHYLLRYKTDSNKQQGSGVSSRFSGSKDISANAGFLLISGVQSDDEADYYCMIWHNSAYVFGGGTKLTVL

SEQ ID NO: 733 - protein, 107 aa, synthetic construct
DIQMTQSPSTLSASVGDRVTITCRASQTIGTWLAWYQQKPGKAPKLLIYEASSLQSGVPSRFSGSGSGTQFTLTISSLQPDDFATYYCQQYNGYFLSFGPGTTVDIR

SEQ ID NO: 734 - protein, 110 aa, synthetic construct
QAGLTQPPSVSKGLRQTATLTCTGNSNNVGNQGATWLQHRQGHPPKLLSYRNNNRPSGISEKLSASRSGSTASLTITGLQPEDEADYYCSAWDSSLSAWVFGGGTKLTVL

SEQ ID NO: 735 - protein, 110 aa, synthetic construct
QARLTQPPSVSEGLRQTATLTCTGNSNNVGHQGVSWVQQHQGHPPKVLSYRTNNRPSGISERFSASRSGNTASLTINGLQPEDEADYYCSAWDKSLNAAVFGGGTRLTVL

SEQ ID NO: 736 - protein, 111 aa, synthetic construct
QAGLTQPPSVSKDLGQTATLTCTGNSNNVGEQGAAWLQQHQGHPPKVLSYRNNNRPSGISERFSASRSANTASLTITGLQPEDEADYYCSAWDTSLSARVFGGGTKLTVLS

SEQ ID NO: 737 - protein, 113 aa, synthetic construct
DIVMTQSPDSLAVSLGERATINCKSSQRIFYRSNNKNYLAWYQQKPGQPPKLLIYWASTRESGVPDRFSGSGSGTDFTLTISSLQAEDVAVYFCQQYYSTPPTFGQGTKVEIK

SEQ ID NO: 738 - protein, 110 aa, synthetic construct
QAGLTQPPSVSKGLRQTATLTCTGNNNNVGNRGAAWLQQRPGHPPKLLSYRNNNRPSGISERFSASRSGNTASLTITGLQPEDEADYYCSAWDNSLSARVFGGGTKLTVL

SEQ ID NO: 739 - protein, 108 aa, synthetic construct
EIVLTQSPATLSLSPGERATLSCRASQSVHSYLAWFQQKPGQAPRLLIYDASNRATGIPARFSGSGSGTDFTLTISSLEPDDFAIYYCQQRYGWPPITFGQGTRLEIK

SEQ ID NO: 740 - protein, 115 aa, synthetic construct
QAVLTQPSSLSASPGASASLTCTLRSGFNVGSYKIYWYQQKPGSPPQFLLKYKSDSDKQQGSGVPSRFSGSKDASANAGILLISGLQSEDEADYYCMIWHSSAVVFGGGTKLTVL

SEQ ID NO: 741 - protein, 111 aa, synthetic construct
QSVLTQPPSVSGAPGRRVTISCAGSSSNIGALYEVHWYQQLPGRAPKLLIYDNTNRPSGVPDRFSGSKSGTSASLAISGLQAEDEGDYYCQSYDSRLSSPVFGGGTKLTVL

SEQ ID NO: 742 - protein, 110 aa, synthetic construct
QTVVTQEPSFSVSPGGTVTVTCGLTSGSVSTINYPSWYRQTPGQPPRTLIYNTNSRSSGVPGRFSGSILGNKAALTITGAQADDESDYYCLLYLGPYTWMFGGGTRVTVL

SEQ ID NO: 743 - protein, 110 aa, synthetic construct
QTVVTQEPSFSVSPGGTVTLTCGLNSDSVSTRHYPSWYQQTPGQAPRTLIYTTNIRSSGVPDRFSASILGNKAALTITGAQADDESTYYCVLYMGRGIWLFGGGTRLTVL

SEQ ID NO: 744 - protein, 112 aa, synthetic construct
QSVLTQPPSVSGAPGQRVTISCTGSSSNIGANYDVHWYQQLPGTAPKLLIYRDNYRPSGVPDRFSGSKSVTSASLAITGLQAEDEGDYYCQSYDDSLGGAIFGGGTQLTVLS

SEQ ID NO: 745 - protein, 107 aa, synthetic construct
SFELTQPSSVSVSPGQTARITCSGDVLAKSFARWLQQKPGQAPILVIYKDTERPSGIPERFSGSSSGTTVTLTISGAQVEDEADYYCYSAADNVQGVFGGGTKLTVL

SEQ ID NO: 746 - protein, 115 aa, synthetic construct
QAVLTQPASLSASPGASASLTCILRSGINVGTYMIYWYQQKPGSPPQFLLRYKSDSDKKQGSGVPSRFSGSKDASANAGILLISGVQSEDEADYYCMIYHSSAWVFGGGTKLTVL

SEQ ID NO: 747 - protein, 113 aa, synthetic construct
QSVLTQPPSVSGAPGQRVTISCTGSSSNIGANYDVHWYQQIPGTAPKLLIYGNINRPSGVPDRFSGSKSGTSASLAITGLQPEDEADYYCQSFDSSLSGQHAVFGGGTHLTVL

SEQ ID NO: 748 - protein, 109 aa, synthetic construct
AIQMTQSPSSLSASVGDRVTITCRASQGINVFLNWYQQKPGKAPKLLIHAASILRTGVPSRFSGSGSGTYFTLTIRGLQPEDFATYYCQQSYGTPPAITFGGGTRIEIK

SEQ ID NO: 749 - protein, 107 aa, synthetic construct
SSELTQPSSVSVSPGQTARITCSGDILAKNYARWFQQKPGQAPVLVIYKDTERPSGIPERFSGSTSGATVTLTISGAQVEDEADYYCYSATDNGLGVFGGGTKLTVL

SEQ ID NO: 750 - protein, 110 aa, synthetic construct
QTVVTQEPSFSVSPGGTVIITCGLRSGSVSASHYPSWYQQTPGQPPRTLIYTTSTRSSGVPDRFSGSILGNKAALTITGAQSDDECDYYCVLYMGNGISVFGGGTKLTVL

SEQ ID NO: 751 - protein, 117 aa, synthetic construct
QSVLTQPPSVSGAPGQRITISCTGTTSNIGAGYDVHWYQQLPGAAPRLLIHGTSSRPSGVPDRFSGSYSGSGSDSGASRYLAITGLRAEDEADYYCQAFDNIVGGWVFGGGTKVTVL

SEQ ID NO: 752 - protein, 112 aa, synthetic construct
QSVLTQPPSVSGAPGQRVTISCTGSSSNIGAGYDVHWYQQLPRVAPKLLIYGNNNRPSGVPDRFSGSKSGTSASLAITGLQAEDEADYYCQSYDSSLSGSFVFGTGTKVTVL

SEQ ID NO: 753 - protein, 106 aa, synthetic construct
KIVLTQSPATLSLSPGERATLSCRASQNVRTYIAWYQHKVGQAPRLLVYDASNRATGIPGRFSGSGSGTDFTLTISSLEPEDFAIYYCQHRGNWLTFGGGTKVEIK

SEQ ID NO: 754 - protein, 110 aa, synthetic construct
QPVLTQTPSVSGPPGQRVTISCSGSSSNMGNNNVYWYQQFPGMAPKLLIYRSSQRPSGVPDRFSGSKSGTSASLAISGLQSEDEAIYHCASWDDSINGWVFGGGTELTVL

SEQ ID NO: 755 - protein, 112 aa, synthetic construct
DIVMTQTPLSSAVTLGQPASFSCMSSQSLVHRDGTTYLTWLQQRPGQPPRLLIYKISNRFSGVPDRFTGSGAGTDFTLKISRVEAEDVGVYYCMQTTQFPYTFGQGTKLEIK

SEQ ID NO: 756 - protein, 107 aa, synthetic construct
AIQLTQSPSSLSASIGDRVTITCRTSQGISSTLAWYQQRPGKPPKLLIYDASTLDDGVPSRFSGSRFGTDFTLTISSLQPEDFATYYCQQFNNYPLTFGGGTKVEIR

SEQ ID NO: 757 - protein, 112 aa, synthetic construct
QSVLTQPPSVSGAPGQRVIISCTGSNSNIGANYDVHWYQQVPGRAPKVLIYGDTNRPSGVPDRFSGSKSGASASLAITGLQAEDEADYYCQSYDRRLSGSIIFGGGTKVTVL

SEQ ID NO: 758 - protein, 107 aa, synthetic construct
SYELTQPSSVSVSPGQTARITCSGNLLAKNFARWFQKKPGQAPVQVLYKDSERPSGIPGRFSGSSSGTTVTLTIRGAQVDDEADYYCYSATDNSLGVFGGGTKLTVL

SEQ ID NO: 759 - protein, 107 aa, synthetic construct
DIQMTQSPSSLSASVGDRVTITCRASQGIRNDLGWYQQKPGQPPERLIYAASNLQSGVPSRFSGSGSGTEFTLTISSLQPEDFATYYCLQHNSYPWTFGQGTKVEIK

SEQ ID NO: 760 - protein, 112 aa, synthetic construct
DIVMTQTPLSSPVTLGQPASISCRASQSLVHSKGDTFLSWFHQRPGQPPRLLIYKVSNRLSGVPDRFSGSGAGTDFTLKISRVEAEDVGVYYCMQNTQYPITFGQGTRLEIK

SEQ ID NO: 761 - protein, 112 aa, synthetic construct
QSVLTQPPSVSGAPGQRVTISCTGSSSNIGAGYDVHWYQQLPGTAPKLLIYVNNNRPSGVPDRFSGSKSGTSASLAITGLQAEDEADYYCQSYDSSLSGSVVFGGGTKLTVL

SEQ ID NO: 762 - protein, 113 aa, synthetic construct
DIVMTQSPDSLAVSLGERATISCKSSQSVFHTSYNRHFIAWYQQKAGQPPKLLIYWSSTRESGVPDRFSGSGSGTDFTLTISSLQAEDVAFYYCQQYHSAPPTFAQGTKVEIK

SEQ ID NO: 763 - protein, 106 aa, synthetic construct
SYELTQPSSVSVSPGQTARITCSGDVLAQKYVRWFQQKPGQAPVLVIYKDTERPSGIPERFSGSISETTVTLTISGAQVEDEADYYCYSATDKWGVFGGGTKLTVL

SEQ ID NO: 764 - protein, 107 aa, synthetic construct
SYELTQPSSVSVSPGQTARITCSGDILAKSYARWFQKKPGQAPVLVIYKDTDRPSGIPERFSGSSSGTTVTLTISGAQVEDEADYYCYSAADNVQGFFGGGTKVTVL

SEQ ID NO: 765 - protein, 113 aa, synthetic construct
DIVMTQSPDSLAVSLGERATIHCKSSQSVLYNSNNKHYIAWTQQKPGQPPKLLIYWASTRESGVPDRFSGSGSGTEFALTINNLQAEDVAVYYCQQYYTIPITFGQGTRLEIK

SEQ ID NO: 766 - protein, 108 aa, synthetic construct
EIVLTQSPGTLSLSPGERATLSCRASQSVTSDFLAWYQQKPDQAPRLLIYSASSRATGIADRFSGSGSGTDFTLTISGLEPEDFALYYCEQYGSTPPTFGQGTKVEIK

SEQ ID NO: 767 - protein, 106 aa, synthetic construct
DIQMTQSPSTLSASVGDRVTITCRASQSISSWLAWYQQKPGKAPKLLIYQASSLESGVPSRFSGSGSGTEFTLTISSLQPDDFATYYCQQYNSYWTFGQGTKVEIK

SEQ ID NO: 768 - protein, 106 aa, synthetic construct
DIQMTQSPSTLSASVGDRVTITCRASQNINSWLAWYQQKPGKAPKLLIYKASSLESGVPSRFSGSGSGPDFTLTISSLQPDDFATYYCHQYYRYWTFGQGTKVEIK

SEQ ID NO: 769 - protein, 108 aa, synthetic construct
SSELTQDPAVSVALGQTVRITCQGDSLRRYDASWYQKKPGQAPVFVMYGKNNRPSGIPDRFSGSSSGNTASLTITGAQAEDEADYYCNSRDSSGYQVVFGGGTKVTVL

SEQ ID NO: 770 - protein, 108 aa, synthetic construct
EIVLTQSPGTLSLSPGERATLSCRASQSVSSSFLAWYQQKPGQAPRLLIFDASSRATGIPDRFSGSGSGTDFSLTISRLEPEDFAVYFCQQYDSSPWTFGQGTKVEIK

SEQ ID NO: 771 - protein, 112 aa, synthetic construct
AVVMTQSPLSLSVTLGQPASISCRSTQSLVCADGHVCLNWFHQRPGQSPRRLIYHVSNRDSGVPDRVSGSGSDTDFTLKISRVEAEDVGVYYCLQGTHWPRTFGQGTKVEIK

SEQ ID NO: 772 - protein, 109 aa, synthetic construct
QTVVTQEPSLTVSPGGTVTLTCASSTGAVTSTYYPNWFQQKPGQAPRALIYGINNKHSWTPARFSGSLLGGKAALTLSGVQPEDEAEYYCLLYSGSGWVFGGGTKVTVL

SEQ ID NO: 773 - protein, 113 aa, synthetic construct
DIVMTQSPDSLAVSLGERATITCKSSQSLLYSSNSQNYLAWFQQKPGQPPRLLIFWAYFRESGVPDRFSGSGSGTDFSLTIGSLQAEDVAVYFCQQYLSFPWTFGQGTKVEIK

SEQ ID NO: 774 - protein, 108 aa, synthetic construct
SYVLTQPPSVSVAPGKTARITCGGNNIRNKNVHWYQQKPGQAPVLVVYDDDDRPSEIPERFSGSNSGNTATLTISRVEAGDEAAYYCQVWDTSSNHVVFGGGTELTVL

SEQ ID NO: 775 - protein, 112 aa, synthetic construct
DIVMTQSPLSLPVTPGEPASISCRSSQSLLHSDGSNYLDWYLQKPGQSPQLLIYLGSNRASGVPDRFSGSGSGTDFTLKISRVEAEDVGVYYCMQTLLVPYTFGQGTKLEIK

SEQ ID NO: 776 - protein, 112 aa, synthetic construct
AIVMTQSPDSLALSLGERATINCKSSQSVLYSPNKKSYLAWYQQKPRQPPKLLIYWASTRKSGVPDRFSGSGSGTDFTLTISSLQAEDVAVYYCQQYFDIPTFGQGTRLEIK

SEQ ID NO: 777 - protein, 109 aa, synthetic construct
EVVLTQSPGTLSLSPGERATLFCRASQSVTTSLGWYQQRPGQPPRLLIYEASNRATGIPARFSGSGSGTDFTLTISSLQPEDFAVYYCQHRNNWPPRYTFGQGTKLEIK

SEQ ID NO: 778 - protein, 109 aa, synthetic construct
QAVVTQEPSVTVSPGGTVTLTCASSAGAVTGGHYPGWFQQKPGQTPRSLIHTTGNKQSWTPARFSGSLLGGKAALTLSAVQPEDEADYYCLLYYGGSPVFGGGTKVTVL

SEQ ID NO: 779 - protein, 111 aa, synthetic construct
QSVLTQPPSVSGTPGQRVIISCSGSSSNTGSTYVYWYQQLPGTAPKLLIYRNDQRPSGVPDRFSGSKSGTSASLAISGLRSEDEADYHCAAWDDSLRGPVFGGGTKLIVLS

SEQ ID NO: 780 - protein, 106 aa, synthetic construct
DVQVTQSPSSLSASVGDRVSITCRASQTLSIAWYQQKPGKAPKLLIYAASRLQSGVPSRFSGSGSGTDFTLTISSLQLEDGATYYCQQSNLTPLLTFGGGTKVEIK

SEQ ID NO: 781 - protein, 115 aa, synthetic construct
VIQLTQSPPSLSASVGDRVTITCRASQYISTYLNWYQQKPGKAPKLLLYAASTLQSGVPSRFSGSGSGTDFTLTISSLQPEDFATYYCQQSYSAPLLSQSRYQFTFGPGTTVDIK

SEQ ID NO: 782 - protein, 110 aa, synthetic construct
QSVLTQPPSASGTPGQRVTISCSGSSSNIGIRNVNWFQQLPGTAPKLLMYNNNQRPSGVPDRFSGSKSGTSASLAINGLQSEDEADYYCSAWDESLKGVIFGGGTKVTVL

SEQ ID NO: 783 - protein, 111 aa, synthetic construct
QAVLTQPPSVSGAPGQRVTISCTGSSSNIGAGFDVHWYQQLPGTAPKLLVYGNNNRPSGVPDRFSASKSGTSASLAIAGLQAEDEADYYCQSYASTLRDVVFGGGTKLTVL

SEQ ID NO: 784 - protein, 106 aa, synthetic construct
SYELSQSLSVSVALGQTASITCGGDNIGTKNVHWYQQRPGQAPVLVMDRDTNRPSGIPERFSGSNSGNTATLSINRAQAGDEADYYCQVWDSNTMIFGGGTKLTVL

SEQ ID NO: 785 - protein, 111 aa, synthetic construct
QSVLTQPPSASGTPGQRVTISCSGSNSNIGSDTVNWYQQLPGTAPKLLIYRNNQRPSGVPDRFSASKSGTSASLAISGLQSEDEADYYCAAWDDSLRGPVFGGGTKLTVLS

SEQ ID NO: 786 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCGSYTSTSTVVFGGGTKLTVL

SEQ ID NO: 787 - protein, 108 aa, synthetic construct
DIQLTQSPSSLSASVGDRVTITCRASRNIGDFLNWYHQKPGKAPKLLIYAASHLQSGVPSRFSGSGSGTYFTLTVSGLQPEDFGTYYCQHSDTSPQITFGQGTRLEIK

SEQ ID NO: 788 - protein, 108 aa, synthetic construct
EIVMTQSPATLSVSPGERATLSCRASQSISRNLAWYQQKPGQAPRLLIYDASTGATGVPARFRGSGSGTEFTLTISSLRSEDFAFYYCQQYYGWPPWTFGQGTKVEIK

SEQ ID NO: 789 - protein, 110 aa, synthetic construct
QSALTQPPSVSGSPGQSVTISCTGSSSDVDIYDHVSWYQQPPGTVPKAVIFEVTNRPSGVPDRFSGSKSGNAASLTISGLQPEDEADYYCTSYTTRDGFFFGTGTKVTVL

SEQ ID NO: 790 - protein, 108 aa, synthetic construct
AYVLTQPPSVSVAPGQTARITCGGNNIGSYSVCWYQQKPGQAPVLVINDDSDRPSGIPERFSGSNSGNTATLTITRVEVGDEADFYCHVWDRGSDQGVFGGGTKLTVL

SEQ ID NO: 791 - protein, 107 aa, synthetic construct
DIQMTQSPSFLPASVGDRVTITCRATQSITNYVNWHRQRPGSAPESLIYSTSLLHFGVPSRFSGDGSGTRFTLTISSLRPEDFATYYCQQTLRVPFTFGGGTTIVAN

SEQ ID NO: 792 - protein, 109 aa, synthetic construct
DIQMTQSPSTLSASVGDRVTITCRASQSITRWLAWYQQKPGKAPKLLIYKASTLESGVPSRFSGSGAGTEFTLTISSLQPDDFATYYCQQYNTYSPPFTFGQGTKLEIK

SEQ ID NO: 793 - protein, 108 aa, synthetic construct
DIQMTQSPSSLSASVGDRVTITCRASQSIATYVNWYQQRPGKAPRLLIYTASNLQSGVPSRFSGSGSGTDFTLTISNLQPEDFATYYCQQSYSTPPYTFGLGTKLEIK

SEQ ID NO: 794 - protein, 108 aa, synthetic construct
SYELTQPSSMSVSPGQTARITCSGDILAKNYARWLQQKPGQAPVLLIHKDRERPSGIPERFSGSSSGTTVTLTISGAQVEDEADYYCYSATDNSLGVFGGGTKLTVLS

SEQ ID NO: 795 - protein, 108 aa, synthetic construct
EILMSQSPAILSVSPGERVTLTCRASESVSTNLAWYQQKPGQAPRLLIYGASTRATGIPARFSGSGSGIEFTLTISSLQSEDLAVYYCQQYNKWFALSFGGGTKVEIK

SEQ ID NO: 796 - protein, 108 aa, synthetic construct
EIVLTQSPGTLSLSPGERATLSCRASQTLSSAYLAWYQQKPGQAPRLLIYGTSRRATGVPDRVRGSGSGTDFTLTISRLEAEDSAVYYCQQYGGSPLTFGGGTKVEMK

SEQ ID NO: 797 - protein, 112 aa, synthetic construct
DIVMTQTPLSSPVTLGQPASISCRSSQSLVHSDGNTYLNWLHQRPGQPPRLLIYKVSNRFSGVPDRFSGSGAGTDFTLKISRVEAEDVGIYYCMQATHFTWTFGQGTKVEIK

SEQ ID NO: 798 - protein, 107 aa, synthetic construct
DIQMTQSPSSLSASVGDRVTITCRASRAISTSLAWYQQKSGKGLRLLIQAASSLQSGVPSRFSGSGSGTDFTLTISGLQPDDFATYYCQQANTFPFTFGPGTKVDIK

SEQ ID NO: 799 - protein, 108 aa, synthetic construct
SYVLTQPPSLSVAPGQTARISCDGDNIGSKSVHWYQQKPGQAPVLVVYDDNDRPSGIPERFSGSNSGNTAILTISRVEAGDEADYSCQVWDSSSAHQVFGGGTKLTVL

SEQ ID NO: 800 - protein, 107 aa, synthetic construct
EIVMTQSPGTLSVSPGERATLSCRASQSVSSSLAWYQQTLGQAPRLLIYGASTRATGIPARFSGSGSGTEFTLTISSLQSEDYAMYYCQQYNNWPYSFGQGTKLEIK

SEQ ID NO: 801 - protein, 107 aa, synthetic construct
DIQMTQSPSALSASVGDRVTITCRASQTIGNYLNWYQQKPGKAPRFLIYDASSLQTGVPSRFSGTGSGTDFSLTISSLQPEDFATYYCQQSYDTPYTFGQGTKLEIR

SEQ ID NO: 802 - protein, 111 aa, synthetic construct
QVELTQSPSASASLGASVKLTCTLSSGHSTLAIAWHQQLPEKGPRYLMRVNSDGSHSKGSGIPDRFAGSSSGAERYLTISSLQSEDEGDYYCQTWDTGIAVFGGGTQVTVL

SEQ ID NO: 803 - protein, 112 aa, synthetic construct
DIVMTQSPDSLAVSLGERATINCKSSQSVLYTSNNKNYLTWYQQKPGQPPKVVIYWASTRESGVPDRFSGSGSGTDFNLTISSLQAEDVAVYYCQQYYHTRTFGQGTKVEIK

SEQ ID NO: 804 - protein, 107 aa, synthetic construct
ETAMTQSPATLSVSPGGRATLTCRASQNVGSNLAWYQRKPGQAPRLLIYGASTRPTGIPARFRGSGSGTEFTLTISSLQSEDFAVYFCQQYNNWPLTFGGGTKVEIK

SEQ ID NO: 805 - protein, 112 aa, synthetic construct
DIVMTQSPLSLSVTPGEPASISCRSSQSLLQTNGNTYLDWYLQKPGQSPQLLITYNGNRASGVPDRFSGSGSGTDFTLTISRVQADDVGVYYCMQAKETPFTFGGGTKVEIK

SEQ ID NO: 806 - protein, 110 aa, synthetic construct
QSALTQPPSASGSPGQSVSISCTGANSDVGGYNYVSWYQQHPGKAPKLLIYEVNKRPSGVPDRFSGSKSGNTASLTVSGLRAEDEADYYCNSYAGSNIWVFGGGTKLTVL

SEQ ID NO: 807 - protein, 108 aa, synthetic construct
DIQMTQSPSTLSASVGDRVTVTCRASENINGWLAWYQQKPGKAPKLLIYQASTLESGVSSRFSGSGSGTQFTLTISRLQSDDVATYYCQHYKGYSQYTFGGGTRVEIR

SEQ ID NO: 808 - protein, 111 aa, synthetic construct
QSVLTQPPSVSGAPGQTVIISCTGSSSNIGAGYDVHWYQQLPGAAPKLLIYESANRPSGVPDRFSGSKSGTSASLAITGLQTEDEADYYCQSYDSGLSGSVFGGGTKLTVL

SEQ ID NO: 809 - protein, 107 aa, synthetic construct
DIQMTQSPSTLSASVGDRVTITCRASQSITTWLSWYQQKPGNAPKLLIYEASNLGFGVPSRFSGSGSGTEFTLTITSLQPDDFATYYCQQHYSYPLSFGGGTKVEIK

SEQ ID NO: 810 - protein, 108 aa, synthetic construct
EIVLTQSPATLSLSPGERATLSCRASQSVSRYLAWYQQKPGQAPRLLIYDVFDRVTGIPARFSGSGSGTDFTLTISSLEPEDSAVYYCQQRGNWPLYTFGQGTKLEIK

SEQ ID NO: 811 - protein, 117 aa, synthetic construct
QPVLTQPTSLSASPGTSARFTCTLRSDIDFVGFRIYWYQQKPGSLPRYLLRYREDSGVERGSGVSSRFSGSKDASTNVGLLIISGLQFEDEADYYCGMWYSKTSEWLIGGGTKLTVL

SEQ ID NO: 812 - protein, 113 aa, synthetic construct
DIVMTQSPDSLAVSLGERASINCRSSQSVLYNSNNKNYLAWYQQKPGQPPKLLIYWASTRESGVPDRFSGSGSGTDFTLTISSLQAEDVAVYHCQQYYSTPVTFGGGTRVEIK

SEQ ID NO: 813 - protein, 109 aa, synthetic construct
EVVLTQSPGTLSLSPGERATLSCRATQSFSSSYLIWYQQKPGQAPRLLIYGASSRATGIPDRFSGSGSGTDFTLTISRLEPEDFAVYYCQHHGPSPPYTFGQGTKLEIR

SEQ ID NO: 814 - protein, 107 aa, synthetic construct
DIQMTQSPSSLSASVGDRVTITCRASQSISTHLNWYQQKPGKAPNLLIFDASSLQRGVPSRFSGVGSGTEFTLTISGLQPEDFATYYCQQSYTTPRTFGQGTKVEVK

SEQ ID NO: 815 - protein, 109 aa, synthetic construct
SYELTQPPSVSVSPGQTARITCSGDALPKQYAYWYQQKPGQAPVLVMYKDLERPSGIPERFSGSSSGTTVALTISGVQAEDEADYYCQSADSSGTYVIFGGGTKLTVLS

SEQ ID NO: 816 - protein, 108 aa, synthetic construct
AIQLTQSPSSLSASVGDRVTITCRASQDISSSLAWYQQNPGKAPKLLIYAASTLQSGVPSRFSGSGSGTDFTLTISSLQPEDFATYYCQHLNSYPLFTFGPGTKVDIK

SEQ ID NO: 817 - protein, 110 aa, synthetic construct
HSALTQPPSASGSPGQSVTISCSGTSSDVGGYNYVSWYQQHPGKAPKLIIYEVNKRPSGVPDRFSGSKSGSTASLTVSGLQAEDEADYYCSSYAGSNNFVFGGGTKLTVL

SEQ ID NO: 818 - protein, 111 aa, synthetic construct
QSVLTQPPSVSAAPGQRVTISCSGSSSNIGNNRVSWYQHLPGTAPKLLIYDNDQRPSGIPDRFSGSKSGTSATLGITGLQTGDEADYYCGTWDSSLSDGRVFGGGTKVTVL

SEQ ID NO: 819 - protein, 107 aa, synthetic construct
SYELTQPSSVSVSPGQTARISCSGPLLSKNFARWFQQKPGQAPILVIYKDTARPSGIPERFSGSSSGTTVTLTISGAQVEDEADYYCYSATDNNLGVFGGGTKLTVL

SEQ ID NO: 820 - protein, 108 aa, synthetic construct
EIVLTQSPGTLSLSPGKRATLSCRASQSISSKYLAWYQQKPGQAPRLLIHAASSRATGIPDRFSGSGSGTDFTLTISRLEPEDFGVYYCQQLGSSPFTFGPGTTVDIK

SEQ ID NO: 821 - protein, 110 aa, synthetic construct
SYELMQPPSVSVSPGQTARITCSGDALPKRYAYWYQQKSGQAPVLVIYEDSKRPSGIPERFSGSSSGTMATLTISGAQVEDEADYYCYSTDSSGSDRRDVFGTGTKVTVL

SEQ ID NO: 822 - protein, 111 aa, synthetic construct
QSVLTQPPSVSEAPGQRVTISCTGSSSNIGAGSDVHWYQQLPGTAPKLLISANSNRPSGVPDRFSGSKSGTSASLVITGLQPEDEADYYCHAYDTSLSASVFGGGTKLTVL

SEQ ID NO: 823 - protein, 110 aa, synthetic construct
QSVLTQPPSVSAAPGQKVTISCSGSSSNIGNTYVSWYQQLPGTAPKLLIYDNDKRPSGIPDRFSGSKSGTSATLGITGLQTGDEADYYCGTWDTSLSAGVFGGGTKLTVL

SEQ ID NO: 824 - protein, 110 aa, synthetic construct
NFMLTQPHSVSESPGKTVTISCTRSSGSIASNYVQWYQQRPGSSPTIVIYEENQRPSGVPDRFSGSIDSSSNSASLTISGLKTEDEADYYCQSYDSSTVVFGGGTKLTVL

SEQ ID NO: 825 - protein, 110 aa, synthetic construct
QSALTQPASVSGSPGQSITISCSGTSSDVGSYNLVSWYQQHPGKAPKLMIYEGSERPSGVSSRFSGSKTDHTASLTISGLQAEDEAHYFCCSYARSRTWVFGGGTKLTVL

SEQ ID NO: 826 - protein, 112 aa, synthetic construct
QSALTQPASVSGSPGQSITISCTGTSSDVGAYNYVSWYQQHPGEAPKLIIYEVTNRPSRVSDRFSGSKSGNTASLIISGLQPGDEADYYCSSYTSKTTLAVFGTGTKVTVVS

SEQ ID NO: 827 - protein, 107 aa, synthetic construct
DIQMTQSPSSLSASIGDRVTITCRASQGISNHLAWFQQKPGKAPKSLIYAASSLQTGVPSKFSGSGSGTDFTLTITSLQPEDFATYYCQQYNSYPYTFGQGTKLDIK

SEQ ID NO: 828 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPHTFGPGTKVDIK

SEQ ID NO: 829 - protein, 111 aa, synthetic construct
QSALTQPASVSGSPGQSITISCTGTSSDVGGFKYVSWYQQHPGKAPKLMIFDVSKRPSGVSNRFSGSKSGNTASLAISGLQAEDEADYYCSSYTTFSSYVVFGGGTKLTVL

SEQ ID NO: 830 - protein, 111 aa, synthetic construct
QSALTQPASVSGSPGQSITISCTGTSSDVGYYNLVSWYQQHPDKAPKLMIFEDSKRPSGVSNRFSGSKSGNTASLTISGLQADDEADYYCSSHAHNGIYVLFGGGTKLTVL

SEQ ID NO: 831 - protein, 107 aa, synthetic construct
SYELTQPPSVSVSPGQTATITCSEDKLGDKYTYWYQQKPGQSPVLVIYQDNKRPSGIPERFSGSSSRNTATLTISGTQPMDEADYHCQAWDSSAVVFGGGTKLTVLS

SEQ ID NO: 832 - protein, 109 aa, synthetic construct
QSVLTQPPSVSGAPGQRVTISCTGSSSNIGAGYDVHWYQQLPGTAPKLLIYGNTNRPSGVPDRFSGSKSGTSASLAITGLQAEDEADYYCQSYDRGLRVFGTGTKVTVL

SEQ ID NO: 833 - protein, 109 aa, synthetic construct
QSVLTQSPSASGTPGQRVTISCSGSSSNIGTNTVTWYQQVPGTAPKVLIYSNNQRPSGVPDRFSGSKSGTSASLAISGLQSEDEADYYCAAWDDSLTVVFGGGTKLTVL

SEQ ID NO: 834 - protein, 108 aa, synthetic construct
SSELTQDPAVSVALGQTVRITCQGDSLRSYYASWYQQKPGQAPVLVIYAKNNRPSGIPDRFSGSTSGNTASLTITGAQAEDEADYYCNSRDSSGNHYVFGTGTKVTVL

SEQ ID NO: 835 - protein, 108 aa, synthetic construct
EIVLTQSPGTLSLSPGERATLSCRASQSVSSADLAWYQQKPGQAPRILIYGASSRATGIPDRFSGSGSGTDFTLTISRLEPEDFAVYYCQQYASSPRTFGQGTKVEIK

SEQ ID NO: 836 - protein, 111 aa, synthetic construct
QSALTQPVSVSGSPGQSITISCTGTSSDIVNYHYVSWYQVHPGKAPKLMIYEVSNRPSGVSDRFSGSRSANTASLTISGLQAEDEADFYCSSYTNSGTLVFGAGTKVTVLS

SEQ ID NO: 837 - protein, 109 aa, synthetic construct
DIVLTQSPATLSLSPGERATLSCRASQSISTYLAWYQQRPGQPPRLLIYNASNRATGIPARFSGSGSGTDFTLTISSLEPEDFAVYYCQQRTNWPPGRTFGQGTKVEIK

SEQ ID NO: 838 - protein, 107 aa, synthetic construct
ETVMTQSPDSLSVSPGARATLSCRTSHSVQSNLAWYQRKPGQPPRLLIYGASTRATGVPARFSGSGSGTEFTLTISGLQSEDFAVYSCQQYNNWPLTFGGGTKVEIR

SEQ ID NO: 839 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSQYTFGQGTKLEIK

SEQ ID NO: 840 - protein, 107 aa, synthetic construct
DIQMTQSPSSLSASVGDRVTITCRASQSISSYLNWYQQKPGKAPKLLIYAASSLHSGVPSRFSGSGSGTDFTLTISSLQPEDFATYYCQQSYSTPWTFGQGTKVEIK

SEQ ID NO: 841 - protein, 112 aa, synthetic construct
DIVMTQSPDSLAVSLGERATINCKSSQSVLYSSNNKHYLAWYQQKPGQPPKLLIYWASTRESGVPDRFSGSGSGTDFTLTISSLQAEDVAVYYCQQYYSTWTFGQGTKVEIK

SEQ ID NO: 842 - protein, 106 aa, synthetic construct
DVQMTQSPTFLSASVGDRVTITCQASQGINNYLNWYQQKPGKAPKLLIYDAFNLEVGVPSRFSGSGSGTEYTFTISSLQPEDIATYYCQQYDNFPTFGQGTKVEIK

SEQ ID NO: 843 - protein, 107 aa, synthetic construct
EIVLTQSPATLSLSPGERATLSCRASQSVSSYLAWYQQKPGQAPRLLIYDTSNRATGIPARFSGSGSGTDFTLTISSLEPEDFAVYYCQQRSNWPITFGQGTRLEIK

SEQ ID NO: 844 - protein, 107 aa, synthetic construct
SSELTQDPSVAVAVGQTVRITCRGTSLRTYYASWYQQKPGQAPLLVIYGKNTRPSGIPDRFSGSSSDNMASLTITGAQAEDEADYFCNSRDSSGRPFFGGGTKVTVL

SEQ ID NO: 845 - protein, 112 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTWTFGQGTKVEIK

SEQ ID NO: 846 - protein, 108 aa, synthetic construct
EIVLTQSPATLSMSPGERATLSCRASQSVSSYLAWYQQKPGQAPRLLIYDASNRATGIPARFSGSGSGTDFTLTISSLEPEDSAVYYCQQRENWPVYTFGQGTRLEIR

SEQ ID NO: 847 - protein, 110 aa, synthetic construct
QSALTQPPSASGSPGQSVSISCTGTSSDVGGYNYVSWYQQHPGQAPKLIIYDVSKRPSGVPDRFSGSKSGNTASLTVSGLQAEDEADYYCSSYAGNKNTVFGGGTKLTVL

SEQ ID NO: 848 - protein, 109 aa, synthetic construct
AIVLTQSPATVSLSPGERATLSCRAFQSAINNDIAWYQQKPRQAPRLVMYGTSNRATGIPDRFRGSGSGADFTLTITRLEPEDYAVYYCQHYGRSKTFTFGPGTRVELK

SEQ ID NO: 849 - protein, 112 aa, synthetic construct
QLMLTQSPSASASLGASVKLTCTLSSGHSTYAIAWLQQRPEKGPRYLMKLNSDGSHDKGDGVPDRFSGSSSGAERSLTISSLQSEDEAEYYCQAWGTGIHGVFGAGTRLTVL

SEQ ID NO: 850 - protein, 107 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNGVFGGGTKLTVL

SEQ ID NO: 851 - protein, 107 aa, synthetic construct
DIQMTQSPSSLSASVGDRVTITCRASQFISSYLNWYQQKPGKAPKILIYAASTLQSGVPSRFSGSGSGTDFTLTISSLQPEDFATYYCQQSSSSPRTFGGGTRVEIK

SEQ ID NO: 852 - protein, 107 aa, synthetic construct
DIQMTQSPSSLSASVGDRVTITCRASQSISNYLNWYQQKPGKVPKLLIYVASSLQSGVPLRFSGSGSGTDFTLTISSLQPEDFATYYCQQSYNAPLTFGQGTKVEIK

SEQ ID NO: 853 - protein, 111 aa, synthetic construct
QSALTQPASVSGSPGQSITISCTGTSSDVGNYNYVSWYQQVPGKAPELLLYDVNNRPSGISNRFSGSKSGNTASLTISGLQADDEADYYCSSYSGSFTPYVFGTGTHVTVQ

SEQ ID NO: 854 - protein, 107 aa, synthetic construct
EIVLTQSPDTLSLSPGERATLSCRASQSVTRSYLAWYQQKPGQAPRLLISGASSRATGVPDRFSGSGSGTDFTLTISRLEPEDLAMYYCQQYGSSHTFGQGTKLEIK

SEQ ID NO: 855 - protein, 107 aa, synthetic construct
DFQVTQSPSTLSASVGDRVTIICRASQSISDWLAWYQQKPGNAPKLLIYKASNLESGVPSRFSGSRSGTEFTLTISSLQPDDFATYYCQLYNSYYYTFGQGTKVEIK

SEQ ID NO: 856 - protein, 107 aa, synthetic construct
EIVMTQSPATLSVSPGERATLSCRASQSISSNLAWYQQKPGQAPRLLIYGASTRATGIPARFSGSGSGTDFTLTISSMQSEDFAVYSCQQYNHWPLTFGGGTKVEIK

SEQ ID NO: 857 - protein, 109 aa, synthetic construct
ETVLTQSPGTLSLSPGESATLSCRASQSVRNNFLAWYQQRPGQAPRLLIYGASSRATGIPDRFSGSGSGTDFTLTISRLEPDDFAVYYCQQYESSPPVTFGPGTRVEIK

SEQ ID NO: 858 - protein, 111 aa, synthetic construct
QSVLTQPPSASGTPGQRITISCSGSSSNIGRNTVNWYQQFPGTAPKLLIHSRNQRPSGVPDRFSGSRSGTSASLAISGLQSEDEADYYCAAWDDSLNGHWVFGGGTKLTVL

SEQ ID NO: 859 - protein, 108 aa, synthetic construct
EIVLTQSPGTLSLSPGERATLSCRASQSVSSNSLAWYQQKPGQAPRLLIYGASSRATDIPDRFSGSGSGTDFTLTIGRLEPEDFAVYYCQQYGSSPWTFGQGTKVEIK

SEQ ID NO: 860 - protein, 108 aa, synthetic construct
DIQLTQSPSRLSASIGDRVTIACRASQNIDVWLAWYQQRPGKAPKLLVYKASTLEPGVPSRFSGSGSGTEFTLTITSLQPDDVATYYCQQYESLSDLTFGGGTKVEIK

SEQ ID NO: 861 - protein, 111 aa, synthetic construct
QLVLTQSPSASASLGASVNLTCALSSGHSSYAIAWHQQKPEKGPRYLMKVNSDGSHSKGDGIPDRFSGSSSGAERYLTISSLQSEDEADYYCQTWGAGIQEFGGGTKLTVL

SEQ ID NO: 862 - protein, 109 aa, synthetic construct
QTVVTQEPSLTVSPGGTVTLTCASSTGAVTSGHYPTWFQQKPGQAPRALIYSTNDKHSRTPARFSGSLLGGKAALTLSGVLPEDEADYYCLLFYGGAWEFGGGTKLTVL

SEQ ID NO: 863 - protein, 110 aa, synthetic construct
QSVLTQPPSVSAAPGQKVTISCSGSSSNIGNNYVSWYQQLPGTAPKLLIYDNNKRPSGIPDRFSGSKSGTSATLGITGLQTEDEADYYCGTWDSSLSAGVFGGGTKLTVL

SEQ ID NO: 864 - protein, 110 aa, synthetic construct
QSALTQARSVSGSPGQSVTISCTGTSSDVGACNCVSWYQQHPGKVPKLIIYDVNKRPSGVPDRFSGSKSGNTASLTISGLQAEDEADYYCCSYAGSYTVLFGGGTTLTVL

SEQ ID NO: 865 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLYSFGQGTKLEIK

SEQ ID NO: 866 - protein, 110 aa, synthetic construct
QSLLTQPPSASGTPGQRVTISCSGSSSNIGTNTVNWYHQLPGTAPKLLIYSDNQRPSGVPDRLSGSKSGTSASLVINGLQSEDEADYYCAAWDDSLKGVIFGGGTKLTVL

SEQ ID NO: 867 - protein, 107 aa, synthetic construct
EVVLTQSPATLSVSPGDSATLSCRASQIVNNNLAWYQQKPGQAPRLLIFASSTRATGIPARFSGSGSGTEFTLTISSLQSEDFAAYYCQQYNYWPGTFGQGTKVEVK

SEQ ID NO: 868 - protein, 108 aa, synthetic construct
ETVLTQSPGTLSLSPGETATLSCRASQSINDNSLAWYHQKPGQAPRLLIFGASTRATDIPDRFSGSGSGTDFTLSISRLEPADFGLYFCQQYDRSPVTFGPGTTVDVK

SEQ ID NO: 869 - protein, 110 aa, synthetic construct
QSALTQPASVSGSPGQSITISCTGTSSDVGTYNLVSWYQQHPGKAPKLMIYEGSKRPSGVSNRFSGSKSGNTASLTISGLQAEDEADYYCCSYSVSSTYVFGTGTKVTAL

SEQ ID NO: 870 - protein, 111 aa, synthetic construct
QSALTQPASVSGSPGQSITISCTGTNNDIGDYYLVSWYQHHPGRAPKLIIYEVTRRPSGTSNRFSGSKSGNTASLTISGLQAEDEADYYCCSFAGGNTFVLFGGGTKVTVL

SEQ ID NO: 871 - protein, 107 aa, synthetic construct
DIQMTQSPSTLSASVGDRVSITCRATQTISNWLAWYQHKPGKAPNLLIYEASDLETGVPSRFSGSGSGTEFTLTISSLQAEDFATYYCQQYKSYPWTFGQGTQVEIK

SEQ ID NO: 872 - protein, 108 aa, synthetic construct
EIVLTQSPGTLSLSPGETATLSCRASQSVGGNYLAWYQHKPGQAPRLVIYGASTRATGIPDRFSGSGSGTDFTLSISRLEPEDFAVYYCQHYGPSPRTFGQGTKVEIK

SEQ ID NO: 873 - protein, 113 aa, synthetic construct
DIVMTQSPDSLAVSLGERATINCKSSQSVLYSFNNKNYLAWYQQKPGQPPKLLIYWASTRESGVPDRFSGSGSGTDFTLTISSLQAEDVAVYYCQQYYSTPLTFGGGTQVEIK

SEQ ID NO: 874 - protein, 112 aa, synthetic construct
QSALTQPRSVSGSPGQSVTISCTGTSIDVGGYNYVSWYQQHPGKAPKVMIYDVSKRPSGVPDRFSGSKSGNTASLTISGLQAEDEADYYCCSYAGTFTFSVVFGGGTKLTVL

SEQ ID NO: 875 - protein, 108 aa, synthetic construct
DIQMTQSPSSLSASVGDRVTITCQASQHINNYLNWYQQKPGKAPKLLIYHASNLETGVPSRFSGSGSGTHFTFTISNLQPEDIATYYCQQFDSLPPPTFGQGTKLDLK

SEQ ID NO: 876 - protein, 107 aa, synthetic construct
DIQLTQSPSFLSASVGDRVSIACRASQGISTYLAWYQQMPGKAPKLLIHSASTLQSGVPSRFSGSGSGTEFTLTITNLQPGDFATYYCQQLNTYPRGFGQGTKVEIR

SEQ ID NO: 877 - protein, 108 aa, synthetic construct
EVVLTQSPGTLSLSPGERATLSCRASQTVTSNFVSWFQQRRGQSPRLLIYGTSSRAAGIPDRFSGSGSGTDFTLTINRLEPEDFAVYYCQQYGSFPITFGPGTKVDIR

SEQ ID NO: 878 - protein, 108 aa, synthetic construct
EVVMTQSPDTLSVSPGERVTLSCRASQSVRSNLAWYQQRRGQAPRLLIYGASTRATGIPARFSGSGSGTEFTLTIDSLQSEDFAIYYCQQYNNWPPWTFGQGTTVEIK

SEQ ID NO: 879 - protein, 107 aa, synthetic construct
DIQMTQSPSSLSASVGDRVTITCRASQDIRNELGWYQQKPGMAPKRLIYDASTLQIGVPSRFSGSGSGTEFTLTISSLQPDDFATYYCLQQNSYPWTFGQGSKVEIK

SEQ ID NO: 880 - protein, 107 aa, synthetic construct
EIVLTQSPATLSLSPGERATLSCRASQGVSRFLAWYQQKPGQAPRLVIYDVSKRATGIPARFSGSGPGPDFTLTISSLEPEDFALYYCQQRSDWPITFGQGTRLEIK

SEQ ID NO: 881 - protein, 110 aa, synthetic construct
QSALTQPPSASGSPGQSVTISCTGTSSDVGGYNYVSWYQHHPGKAPKLMICDVNKRPSGVPDRFSGSKSGNTASLTVSGLQAEDEADYYCSSYAGSNNLVFGGGTKLTVL

SEQ ID NO: 882 - protein, 109 aa, synthetic construct
QTVVTQEPSLTVSPGGTVTLTCASSTGAVTSGYYPNWFQQKPGQAPRPLIYSTSNRHSWTPARFSGSLLGGKAALTLSGVQPEDEAEYYCLLYCGGPMVFGGGTKLTVL

SEQ ID NO: 883 - protein, 108 aa, synthetic construct
SYELTQPPSVSVSPGQTARITCSGDALPKRYAYWYLQKPGQAPVLVIYKDTERPSGIPERFSGSSSGTTVTLTISGVQAEDEADYYCQSADSSGTHVFFGGGTKLTVL

SEQ ID NO: 884 - protein, 107 aa, synthetic construct
SYELTQPPSVSVSPGQTASITCSGDKLGDKYACWYQQKPGQSPVLVIYQDSKRPSGIPERFSGSNSGNTATLTISGTQAMDEADYYCQAWDSSTGVVFGGGTKLTVL

SEQ ID NO: 885 - protein, 108 aa, synthetic construct
DIQMTQSPFSLSASVGDRVTISCRAGQNIGTFLNWYQHKPGQAPKLLIYSASRLRSGVPSRFSGSGSGTNFALTISSLRPEDLATYYCQNSDSAPSLTFGGGTRVEIK

SEQ ID NO: 886 - protein, 111 aa, synthetic construct
QSALTQPPSASGSPGQSVTISCTGTSSDVGGYNYVSWYQQHPGKAPKLMIYEVSKRPSGVPDRFSGSKSGNTASLTVSGLQAEDEADYYCSSHAGSNNLGVFGTGTKVTVL

SEQ ID NO: 887 - protein, 110 aa, synthetic construct
QSALTQPASMSGSPGQSITISCTGTSSDVGNYNYVSWYQQHPGKAPKLMIYDVSNRPSGVSNRFSGSKSGNTASLTISGLQAEDEADYYCTSYTSISTYLFGTGTRVTVL

SEQ ID NO: 888 - protein, 110 aa, synthetic construct
QSVLTQPPSASGTPGQRVTISCSGSSSNIGSNFVYWYQQLPGTAPKLLIYRNNQRPSGVPDRFSGSTSGTSASLAISGLRSEDEADYYCAAWDDSLSGWVFGGGTKLTVL

SEQ ID NO: 889 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTRLEIK

SEQ ID NO: 890 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIK

SEQ ID NO: 891 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPHTFGQGTKVEIK

SEQ ID NO: 892 - protein, 108 aa, synthetic construct
DIQMTQSPSSLSASVGDRVTITCRASRSISSSLNWYQQKPGKAPKLLIYAASSLQSGVPSRFSGSGSGTDFTLTISSLQPEDFATYYCQQSYNTPLFTFGPGTKVDIK

SEQ ID NO: 893 - protein, 110 aa, synthetic construct
NFMLTQPHSVSESPGKTVTISCTRSGGRIDNNYVQWYQQRPGSAPTMVIYEDNQRPSGVPDRFSGSIDSSSNSASLTISGLKTEDEADYYCQSYDSDFWVFGGGTKLTVL

SEQ ID NO: 894 - protein, 111 aa, synthetic construct
QAVVTQEPSLTVSPGGTVTLTCGSSTGAVTSGHYPYWFQQKPGQAPTTLIYDTNNKASWTPARFSGSLLGDKAALTLSGAQPDDEADYYCLLCHNGARDWVFGGGTKLTVL

SEQ ID NO: 895 - protein, 109 aa, synthetic construct
QAVVTQEPSVTVSPGGTVILTCGSSTGAVTSGHYPYWFQQKPGQAPRTLIYDTNNKYSWTPARFSGSLLGGKAALTLSGAQPEDEAEYYCLFWYSYVGLIGGGTRLTVL

SEQ ID NO: 896 - protein, 111 aa, synthetic construct
QSVLTQPPSVSGAPGQRVTISCTGSSSNIGANYNVHWYQQFPGTAPKLLIYDNVNRPSGVPDRFSGSKSGTSASLAITGLRVEDESDYYCQSYDSSLSGWVFGGGTKLTVL

SEQ ID NO: 897 - protein, 106 aa, synthetic construct
SYELTQPPSVSVSPGQTASITCSGDKLGNKYACWYQQKPGQSPLLVIYEDNKRPSGIPERFSGSNSGNTATLTISGTQAMDEADYYCQAWDNRLWVFGGGTKLTVL

SEQ ID NO: 898 - protein, 111 aa, synthetic construct
DIVLTQSPLSLPVTPGEPASISCRSSQSLLHSNGYNYLNWYLQKPGQSPQLLIYLASNRASGVPDRFSGSGSGTHFTLKISRVEAEDVGVYYCMQARLSYTFGQGTKLSIK

SEQ ID NO: 899 - protein, 107 aa, synthetic construct
DIQMTQSPSSLSASVGDRVTITCRASQGIRNHLAWYQQKPGKVPKLLIYAASTLQSGVPSRFSGSGSGTDFTLTISSLQPEDVATYYCQKCHSAPYTFGQGTKVEIK

SEQ ID NO: 900 - protein, 106 aa, synthetic construct
DILMTQSPSSLSASVGDRVTITCQASHDINNYLNWYQQKPGKAPKLIYDASNLETGVASRFSGSGSGTEFTLTIRSLQPEDFATYYCQQYESLPITFGQGTRLEIK

SEQ ID NO: 901 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSYPYTFGQGTRLEIK

SEQ ID NO: 902 - protein, 109 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPPLFTFGPGTKVDIK

SEQ ID NO: 903 - protein, 111 aa, synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQSWTFGQGTKVEIK

SEQ ID NO: 904 - protein, 116 aa, synthetic construct
QPVLTQPPSASASLGASVTLTCTLSGGYSNYKVDWYQQRPGKGPRFVMRVGTGGIVGSKGDGIPDRFSVLGSGLNRYLTIKNIQEEDESDYHCGADHGSGSNFVWVFGGGTKLTVL

SEQ ID NO: 905 - protein, 106 aa, synthetic construct
SYELTQPPSVSVSPGQTASITCSGDKLGDKYACWYQQKPGQSPVLLIYQDSKRPSGIPERFSGSNSGNTATLTISGTQAMDEADYYCQAWDSSTAVFGGGTKLTVL

SEQ ID NO: 906 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPFTFGPGTKVDIK

SEQ ID NO: 907 - protein, 104 aa, synthetic construct
QSALTQPASMSGSPGQSITISCTLTDEHHFVSWYQQHPGKVPKLLIYDAIHRSSGFSGRFSASQSGNTASLTISGLRFEDEADYYCSTYRGDVIFGGGTKLTVL

SEQ ID NO: 908 - protein, 107 aa, synthetic construct
AIQLTQSPSSLSASVGDRVTITCRASQDISSALAWYQQKPGKPPKVLIFDASTLESGVPSRFSGSGSGTDFTLTITSLQPEDFAAYYCQQFNSYPRTFGQGTRLEIK

SEQ ID NO: 909 - protein, 107 aa, synthetic construct
EIVLTQSPGTLSLSPGERATLSCRASQSVSSDLAWYQQKPGQAPRLVIYDAFNRATGIPARFSGSGSGTDFTLTISRLEPEDFAVYYCQQRNKWPLTFGGGTKVEIK

SEQ ID NO: 910 - protein, 107 aa, synthetic construct
DIQMTQSPSSLSASVGDRVTITCRASQSISRYLNWYQQKPGKAPKLMIFAISTLQSGVPSRFSGSGSGTDFTLTISSLQPEDFATYYCQQSYRTPDTFGQGTKLELK

SEQ ID NO: 911 - protein, 110 aa, synthetic construct
NFKLTQPHSVSGSPGKTVRISCTRSSGNIATNGVQWYQQRPGSSPTTLIYEDNERPFGVPDRFSGSIDTSSNSASLTISGLKTEDEADYYCQSYEDNYLVFGGGTKLTVL

SEQ ID NO: 912 - protein, 106 aa, synthetic construct
SYELTQPPSVSVSPGQTASITCSGDKLGDKYACWYQQKPGQSPVLVIYQDTKRPSGIPERFSGSNSGNTATLTISGTQAMDEADYYCQAWDSSTAVFGGGTQLTVL

SEQ ID NO: 913 - protein, 110 aa, synthetic construct
ETVLTQSPATLSLSPGERATLSCRASQSVRSNLAWYQHKPGQAPRLLIYDASNRATGIPARFSGSGSGTDFTLTISSLEPEDFAVYYCQQRNNWPPGEVTFGGGTKVEIK

SEQ ID NO: 914 - protein, 108 aa, synthetic construct
GIVLTQSPGTLSLSPGDRATLSCRASQTFNSAYLAWYQQKPGQPPRLLIYSASARASGTPDRFSGSESGTVFTLTISRLAPEDFAVYYCHQYHLSPFTFGPGTKVEIN

SEQ ID NO: 915 - protein, 110 aa, synthetic construct
SSELTQDPAVSAALGQTVRITCRGDSLRKYYASWYQLKPGQAPVLVIYRKNSRPSGIPDRFSGSSSGNTASLTITGAQAEDEADYYCNSRDNSGHHLYVFGTGTKVTVLS

SEQ ID NO: 916 - protein, 108 aa, synthetic construct
SYVLTQPPSLSVAPGETAWITCGGNNLGSKSVHWHQQKPGQAPVLVIYYDSDRPSGIPERFTGSKSGNTATLTISRVEAGDEADYYCQVWDRSSDHVVFGGGTKLTVL

SEQ ID NO: 917 - protein, 113 aa, synthetic construct
DIVMTQSPDSLAVSLGERATINCKSSQSVLYSSNNKKYLAWYQQKPGQPPKLLIYWASTRESGVPDRFSGSGSGTDFTLTISSLQAEDVAVYYCQQYYSSSYTFGQGTKLEIK

SEQ ID NO: 918 - protein, 108 aa, synthetic construct
SSELTQDPAVSVALGQTVRITCQGDSLRRYYATWYQQKPGQAPVLVIYGKNNRPSGIPDRFSGSSSGNTASLTITGAQAEDEADYYCNSRDSSANHEVFGGGTKLTVL

SEQ ID NO: 919 - protein, 113 aa, synthetic construct
DIVVTQSPESLAVSLGERATINCKSSQSVLYSPNSKNYLAWYQQRPGQPPKLLIYWASTRESGVPERFSGSGSGTDFALTISSLQAEDVGIYYCQQYMIRPFAFGQGTKLEIK

SEQ ID NO: 920 - protein, 108 aa, synthetic construct
SYELTQPPSVSVSPGQTARITCSGDALPKQYAYWYQQKPGQAPMLVIYKDSERPSGIPERFSGSSSGTTVTLTISGVQAEDEADYYCQSADISGTHVLFGGGTKLTVL

SEQ ID NO: 921 - protein, 108 aa, synthetic construct
EIVMTQSPATLSVSPGERATLSCRASQSVTSNLAWYQQKPGQAPRLLIYGASTRATGIPGRFSGGGSGTEFTLTISSLQSEDFAVYYCQHYNSWPPWTFGQGTKVEIK

SEQ ID NO: 922 - protein, 107 aa, synthetic construct
EIVLTQSPATLSLSPGERATLSCGASQSVSMNYLAWYQQKPGLAPRLLIYDASSRPSGIPDRFSGSGSGTDFTLTISRLEPEDFAVYYCQQYGSSGAFGPGTKVDIK

SEQ ID NO: 923 - protein, 4 aa, synthetic construct
NPNA

SEQ ID NO: 924 - protein, 4 aa, synthetic construct
NANP

SEQ ID NO: 925 - protein, 4 aa, synthetic construct
ANPN

SEQ ID NO: 926 - protein, 4 aa, synthetic construct
PNAN

SEQ ID NO: 927 - protein, 5 aa, synthetic construct
NANPN

SEQ ID NO: 928 - protein, 5 aa, synthetic construct
ANPNA

SEQ ID NO: 929 - protein, 5 aa, synthetic construct
NPNAN

SEQ ID NO: 930 - protein, 5 aa, synthetic construct
PNANP

SEQ ID NO: 931 - protein, 6 aa, synthetic construct
NANPNA

SEQ ID NO: 932 - protein, 6 aa, synthetic construct
ANPNAN

SEQ ID NO: 933 - protein, 6 aa, synthetic construct
NPNANP

SEQ ID NO: 934 - protein, 6 aa, synthetic construct
PNANPN

SEQ ID NO: 935 - protein, 7 aa, synthetic construct
NANPNAN

SEQ ID NO: 936 - protein, 7 aa, synthetic construct
ANPNANP

SEQ ID NO: 937 - protein, 7 aa, synthetic construct
NPNANPN

SEQ ID NO: 938 - protein, 7 aa, synthetic construct
PNANPNA

SEQ ID NO: 939 - protein, 8 aa, synthetic construct
NANPNANP

SEQ ID NO: 940 - protein, 8 aa, synthetic construct
ANPNANPN

SEQ ID NO: 941 - protein, 8 aa, synthetic construct
NPNANPNA

SEQ ID NO: 942 - protein, 8 aa, synthetic construct
PNANPNAN

SEQ ID NO: 943 - protein, 9 aa, synthetic construct
NANPNANPN

SEQ ID NO: 944 - protein, 9 aa, synthetic construct
ANPNANPNA

SEQ ID NO: 945 - protein, 9 aa, synthetic construct
NPNANPNAN

SEQ ID NO: 946 - protein, 9 aa, synthetic construct
PNANPNANP

SEQ ID NO: 947 - protein, 10 aa, synthetic construct
NANPNANPNA

SEQ ID NO: 948 - protein, 10 aa, synthetic construct
ANPNANPNAN

SEQ ID NO: 949 - protein, 10 aa, synthetic construct
NPNANPNANP

SEQ ID NO: 950 - protein, 10 aa, synthetic construct
PNANPNANPN

SEQ ID NO: 951 - protein, 11 aa, synthetic construct
NANPNANPNAN

SEQ ID NO: 952 - protein, 11 aa, synthetic construct
ANPNANPNANP

SEQ ID NO: 953 - protein, 11 aa, synthetic construct
NPNANPNANPN

SEQ ID NO: 954 - protein, 11 aa, synthetic construct
PNANPNANPNA

SEQ ID NO: 955 - protein, 12 aa, synthetic construct
NANPNANPNANP

SEQ ID NO: 956 - protein, 12 aa, synthetic construct
ANPNANPNANPN

SEQ ID NO: 957 - protein, 12 aa, synthetic construct
NPNANPNANPNA

SEQ ID NO: 958 - protein, 12 aa, synthetic construct
PNANPNANPNAN

SEQ ID NO: 959 - protein, 13 aa, synthetic construct
NANPNANPNANPN

SEQ ID NO: 960 - protein, 13 aa, synthetic construct
ANPNANPNANPNA

SEQ ID NO: 961 - protein, 13 aa, synthetic construct
NPNANPNANPNAN

SEQ ID NO: 962 - protein, 13 aa, synthetic construct
PNANPNANPNANP

SEQ ID NO: 963 - protein, 14 aa, synthetic construct
NANPNANPNANPNA

SEQ ID NO: 964 - protein, 14 aa, synthetic construct
ANPNANPNANPNAN

SEQ ID NO: 965 - protein, 14 aa, synthetic construct
NPNANPNANPNANP

SEQ ID NO: 966 - protein, 14 aa, synthetic construct
PNANPNANPNANPN

SEQ ID NO: 967 - protein, 15 aa, synthetic construct
NANPNANPNANPNAN

SEQ ID NO: 968 - protein, 15 aa, synthetic construct
ANPNANPNANPNANP

SEQ ID NO: 969 - protein, 15 aa, synthetic construct
NPNANPNANPNANPN

SEQ ID NO: 970 - protein, 15 aa, synthetic construct
PNANPNANPNANPNA

SEQ ID NO: 971 - protein, 16 aa, synthetic construct
NANPNANPNANPNANP

SEQ ID NO: 972 - protein, 16 aa, synthetic construct
ANPNANPNANPNANPN

SEQ ID NO: 973 - protein, 16 aa, synthetic construct
NPNANPNANPNANPNA

SEQ ID NO: 974 - protein, 16 aa, synthetic construct
PNANPNANPNANPNAN

SEQ ID NO: 975 - protein, 4 aa, synthetic construct
ADGN

SEQ ID NO: 976 - protein, 4 aa, synthetic construct
DGNP

SEQ ID NO: 977 - protein, 4 aa, synthetic construct
DPNA

SEQ ID NO: 978 - protein, 4 aa, synthetic construct
GNPD

SEQ ID NO: 979 - protein, 4 aa, synthetic construct
NPDP

SEQ ID NO: 980 - protein, 4 aa, synthetic construct
NPNV

SEQ ID NO: 981 - protein, 4 aa, synthetic construct
NVDP

SEQ ID NO: 982 - protein, 4 aa, synthetic construct
PADG

SEQ ID NO: 983 - protein, 4 aa, synthetic construct
PDPN

SEQ ID NO: 984 - protein, 4 aa, synthetic construct
PNVD

SEQ ID NO: 985 - protein, 4 aa, synthetic construct
VDPN

SEQ ID NO: 986 - protein, 5 aa, synthetic construct
ADGNP

SEQ ID NO: 987 - protein, 5 aa, synthetic construct
ANPNV

SEQ ID NO: 988 - protein, 5 aa, synthetic construct
DGNPD

SEQ ID NO: 989 - protein, 5 aa, synthetic construct
DPNAN

SEQ ID NO: 990 - protein, 5 aa, synthetic construct
GNPDP

SEQ ID NO: 991 - protein, 5 aa, synthetic construct
NPDPN

SEQ ID NO: 992 - protein, 5 aa, synthetic construct
NPNVD

SEQ ID NO: 993 - protein, 5 aa, synthetic construct
NVDPN

SEQ ID NO: 994 - protein, 5 aa, synthetic construct
PADGN

SEQ ID NO: 995 - protein, 5 aa, synthetic construct
PDPNA

SEQ ID NO: 996 - protein, 5 aa, synthetic construct
PNVDP

SEQ ID NO: 997 - protein, 5 aa, synthetic construct
VDPNA

SEQ ID NO: 998 - protein, 6 aa, synthetic construct
ADGNPD

SEQ ID NO: 999 - protein, 6 aa, synthetic construct
ANPNVD

SEQ ID NO: 1000 - protein, 6 aa, synthetic construct
DGNPDP

SEQ ID NO: 1001 - protein, 6 aa, synthetic construct
DPNANP

SEQ ID NO: 1002 - protein, 6 aa, synthetic construct
GNPDPN

SEQ ID NO: 1003 - protein, 6 aa, synthetic construct
NANPNV

SEQ ID NO: 1004 - protein, 6 aa, synthetic construct
NPDPNA

SEQ ID NO: 1005 - protein, 6 aa, synthetic construct
NPNVDP

SEQ ID NO: 1006 - protein, 6 aa, synthetic construct
NVDPNA

SEQ ID NO: 1007 - protein, 6 aa, synthetic construct
PADGNP

SEQ ID NO: 1008 - protein, 6 aa, synthetic construct
PDPNAN

SEQ ID NO: 1009 - protein, 6 aa, synthetic construct
PNVDPN

SEQ ID NO: 1010 - protein, 6 aa, synthetic construct
VDPNAN

SEQ ID NO: 1011 - protein, 7 aa, synthetic construct
ADGNPDP

SEQ ID NO: 1012 - protein, 7 aa, synthetic construct
ANPNVDP

SEQ ID NO: 1013 - protein, 7 aa, synthetic construct
DGNPDPN

SEQ ID NO: 1014 - protein, 7 aa, synthetic construct
DPNANPN

SEQ ID NO: 1015 - protein, 7 aa, synthetic construct
GNPDPNA

SEQ ID NO: 1016 - protein, 7 aa, synthetic construct
NANPNVD

SEQ ID NO: 1017 - protein, 7 aa, synthetic construct
NPDPNAN

SEQ ID NO: 1018 - protein, 7 aa, synthetic construct
NPNVDPN

SEQ ID NO: 1019 - protein, 7 aa, synthetic construct
NVDPNAN

SEQ ID NO: 1020 - protein, 7 aa, synthetic construct
PADGNPD

SEQ ID NO: 1021 - protein, 7 aa, synthetic construct
PDPNANP

SEQ ID NO: 1022 - protein, 7 aa, synthetic construct
PNANPNV

SEQ ID NO: 1023 - protein, 7 aa, synthetic construct
PNVDPNA

SEQ ID NO: 1024 - protein, 7 aa, synthetic construct
VDPNANP

SEQ ID NO: 1025 - protein, 8 aa, synthetic construct
ADGNPDPN

SEQ ID NO: 1026 - protein, 8 aa, synthetic construct
ANPNVDPN

SEQ ID NO: 1027 - protein, 8 aa, synthetic construct
DGNPDPNA

SEQ ID NO: 1028 - protein, 8 aa, synthetic construct
DPNANPNA

SEQ ID NO: 1029 - protein, 8 aa, synthetic construct
DPNANPNV

SEQ ID NO: 1030 - protein, 8 aa, synthetic construct
GNPDPNAN

SEQ ID NO: 1031 - protein, 8 aa, synthetic construct
NANPNVDP

SEQ ID NO: 1032 - protein, 8 aa, synthetic construct
NPDPNANP

SEQ ID NO: 1033 - protein, 8 aa, synthetic construct
NPNVDPNA

SEQ ID NO: 1034 - protein, 8 aa, synthetic construct
NVDPNANP

SEQ ID NO: 1035 - protein, 8 aa, synthetic construct
PADGNPDP

SEQ ID NO: 1036 - protein, 8 aa, synthetic construct
PDPNANPN

SEQ ID NO: 1037 - protein, 8 aa, synthetic construct
PNANPNVD

SEQ ID NO: 1038 - protein, 8 aa, synthetic construct
PNVDPNAN

SEQ ID NO: 1039 - protein, 8 aa, synthetic construct
VDPNANPN

SEQ ID NO: 1040 - protein, 9 aa, synthetic construct
ADGNPDPNA

SEQ ID NO: 1041 - protein, 9 aa, synthetic construct
ANPNVDPNA

SEQ ID NO: 1042 - protein, 9 aa, synthetic construct
DGNPDPNAN

SEQ ID NO: 1043 - protein, 9 aa, synthetic construct
DPNANPNAN

SEQ ID NO: 1044 - protein, 9 aa, synthetic construct
DPNANPNVD

SEQ ID NO: 1045 - protein, 9 aa, synthetic construct
GNPDPNANP

SEQ ID NO: 1046 - protein, 9 aa, synthetic construct
NANPNVDPN

SEQ ID NO: 1047 - protein, 9 aa, synthetic construct
NPDPNANPN

SEQ ID NO: 1048 - protein, 9 aa, synthetic construct
NPNVDPNAN

SEQ ID NO: 1049 - protein, 9 aa, synthetic construct
NVDPNANPN

SEQ ID NO: 1050 - protein, 9 aa, synthetic construct
PADGNPDPN

SEQ ID NO: 1051 - protein, 9 aa, synthetic construct
PDPNANPNV

SEQ ID NO: 1052 - protein, 9 aa, synthetic construct
PNANPNVDP

SEQ ID NO: 1053 - protein, 9 aa, synthetic construct
PNVDPNANP

SEQ ID NO: 1054 - protein, 9 aa, synthetic construct
VDPNANPNA

SEQ ID NO: 1055 - protein, 9 aa, synthetic construct
VDPNANPNV

SEQ ID NO: 1056 - protein, 10 aa, synthetic construct
ADGNPDPNAN

SEQ ID NO: 1057 - protein, 10 aa, synthetic construct
ANPNVDPNAN

SEQ ID NO: 1058 - protein, 10 aa, synthetic construct
DGNPDPNANP

SEQ ID NO: 1059 - protein, 10 aa, synthetic construct
DPNANPNANP

SEQ ID NO: 1060 - protein, 10 aa, synthetic construct
DPNANPNVDP

SEQ ID NO: 1061 - protein, 10 aa, synthetic construct
GNPDPNANPN

SEQ ID NO: 1062 - protein, 10 aa, synthetic construct
NANPNVDPNA

SEQ ID NO: 1063 - protein, 10 aa, synthetic construct
NPDPNANPNV

SEQ ID NO: 1064 - protein, 10 aa, synthetic construct
NPNVDPNANP

SEQ ID NO: 1065 - protein, 10 aa, synthetic construct
NVDPNANPNA

SEQ ID NO: 1066 - protein, 10 aa, synthetic construct
NVDPNANPNV

SEQ ID NO: 1067 - protein, 10 aa, synthetic construct
PADGNPDPNA

SEQ ID NO: 1068 - protein, 10 aa, synthetic construct
PDPNANPNVD

SEQ ID NO: 1069 - protein, 10 aa, synthetic construct
PNANPNVDPN

SEQ ID NO: 1070 - protein, 10 aa, synthetic construct
PNVDPNANPN

SEQ ID NO: 1071 - protein, 10 aa, synthetic construct
VDPNANPNAN

SEQ ID NO: 1072 - protein, 10 aa, synthetic construct
VDPNANPNVD

SEQ ID NO: 1073 - protein, 11 aa, synthetic construct
ADGNPDPNANP

SEQ ID NO: 1074 - protein, 11 aa, synthetic construct
ANPNVDPNANP

SEQ ID NO: 1075 - protein, 11 aa, synthetic construct
DGNPDPNANPN

SEQ ID NO: 1076 - protein, 11 aa, synthetic construct
DPNANPNANPN

SEQ ID NO: 1077 - protein, 11 aa, synthetic construct
DPNANPNVDPN

SEQ ID NO: 1078 - protein, 11 aa, synthetic construct
GNPDPNANPNV

SEQ ID NO: 1079 - protein, 11 aa, synthetic construct
NANPNVDPNAN

SEQ ID NO: 1080 - protein, 11 aa, synthetic construct
NPDPNANPNVD

SEQ ID NO: 1081 - protein, 11 aa, synthetic construct
NPNVDPNANPN

SEQ ID NO: 1082 - protein, 11 aa, synthetic construct
NVDPNANPNAN

SEQ ID NO: 1083 - protein, 11 aa, synthetic construct
NVDPNANPNVD

SEQ ID NO: 1084 - protein, 11 aa, synthetic construct
PADGNPDPNAN

SEQ ID NO: 1085 - protein, 11 aa, synthetic construct
PDPNANPNVDP

SEQ ID NO: 1086 - protein, 11 aa, synthetic construct
PNANPNVDPNA

SEQ ID NO: 1087 - protein, 11 aa, synthetic construct
PNVDPNANPNA

SEQ ID NO: 1088 - protein, 11 aa, synthetic construct
PNVDPNANPNV

SEQ ID NO: 1089 - protein, 11 aa, synthetic construct
VDPNANPNANP

SEQ ID NO: 1090 - protein, 11 aa, synthetic construct
VDPNANPNVDP

SEQ ID NO: 1091 - protein, 12 aa, synthetic construct
ADGNPDPNANPN

SEQ ID NO: 1092 - protein, 12 aa, synthetic construct
ANPNVDPNANPN

SEQ ID NO: 1093 - protein, 12 aa, synthetic construct
DGNPDPNANPNV

SEQ ID NO: 1094 - protein, 12 aa, synthetic construct
DPNANPNANPNA

SEQ ID NO: 1095 - protein, 12 aa, synthetic construct
DPNANPNVDPNA

SEQ ID NO: 1096 - protein, 12 aa, synthetic construct
GNPDPNANPNVD

SEQ ID NO: 1097 - protein, 12 aa, synthetic construct
NANPNVDPNANP

SEQ ID NO: 1098 - protein, 12 aa, synthetic construct
NPDPNANPNVDP

SEQ ID NO: 1099 - protein, 12 aa, synthetic construct
NPNVDPNANPNA

SEQ ID NO: 1100 - protein, 12 aa, synthetic construct
NPNVDPNANPNV

SEQ ID NO: 1101 - protein, 12 aa, synthetic construct
NVDPNANPNANP

SEQ ID NO: 1102 - protein, 12 aa, synthetic construct
NVDPNANPNVDP

SEQ ID NO: 1103 - protein, 12 aa, synthetic construct
PADGNPDPNANP

SEQ ID NO: 1104 - protein, 12 aa, synthetic construct
PDPNANPNVDPN

SEQ ID NO: 1105 - protein, 12 aa, synthetic construct
PNANPNVDPNAN

SEQ ID NO: 1106 - protein, 12 aa, synthetic construct
PNVDPNANPNAN

SEQ ID NO: 1107 - protein, 12 aa, synthetic construct
PNVDPNANPNVD

SEQ ID NO: 1108 - protein, 12 aa, synthetic construct
VDPNANPNANPN

SEQ ID NO: 1109 - protein, 12 aa, synthetic construct
VDPNANPNVDPN

SEQ ID NO: 1110 - protein, 13 aa, synthetic construct
ADGNPDPNANPNV

SEQ ID NO: 1111 - protein, 13 aa, synthetic construct
ANPNVDPNANPNA

SEQ ID NO: 1112 - protein, 13 aa, synthetic construct
ANPNVDPNANPNV

SEQ ID NO: 1113 - protein, 13 aa, synthetic construct
DGNPDPNANPNVD

SEQ ID NO: 1114 - protein, 13 aa, synthetic construct
DPNANPNANPNAN

SEQ ID NO: 1115 - protein, 13 aa, synthetic construct
DPNANPNVDPNAN

SEQ ID NO: 1116 - protein, 13 aa, synthetic construct
GNPDPNANPNVDP

SEQ ID NO: 1117 - protein, 13 aa, synthetic construct
NANPNVDPNANPN

SEQ ID NO: 1118 - protein, 13 aa, synthetic construct
NPDPNANPNVDPN

SEQ ID NO: 1119 - protein, 13 aa, synthetic construct
NPNVDPNANPNAN

SEQ ID NO: 1120 - protein, 13 aa, synthetic construct
NPNVDPNANPNVD

SEQ ID NO: 1121 - protein, 13 aa, synthetic construct
NVDPNANPNANPN

SEQ ID NO: 1122 - protein, 13 aa, synthetic construct
NVDPNANPNVDPN

SEQ ID NO: 1123 - protein, 13 aa, synthetic construct
PADGNPDPNANPN

SEQ ID NO: 1124 - protein, 13 aa, synthetic construct
PDPNANPNVDPNA

SEQ ID NO: 1125 - protein, 13 aa, synthetic construct
PNANPNVDPNANP

SEQ ID NO: 1126 - protein, 13 aa, synthetic construct
PNVDPNANPNANP

SEQ ID NO: 1127 - protein, 13 aa, synthetic construct
PNVDPNANPNVDP

SEQ ID NO: 1128 - protein, 13 aa, synthetic construct
VDPNANPNANPNA

SEQ ID NO: 1129 - protein, 13 aa, synthetic construct
VDPNANPNVDPNA

SEQ ID NO: 1130 - protein, 14 aa, synthetic construct
ADGNPDPNANPNVD

SEQ ID NO: 1131 - protein, 14 aa, synthetic construct
ANPNVDPNANPNAN

SEQ ID NO: 1132 - protein, 14 aa, synthetic construct
ANPNVDPNANPNVD

SEQ ID NO: 1133 - protein, 14 aa, synthetic construct
DGNPDPNANPNVDP

SEQ ID NO: 1134 - protein, 14 aa, synthetic construct
DPNANPNANPNANP

SEQ ID NO: 1135 - protein, 14 aa, synthetic construct
DPNANPNVDPNANP

SEQ ID NO: 1136 - protein, 14 aa, synthetic construct
GNPDPNANPNVDPN

SEQ ID NO: 1137 - protein, 14 aa, synthetic construct
NANPNVDPNANPNA

SEQ ID NO: 1138 - protein, 14 aa, synthetic construct
NANPNVDPNANPNV

SEQ ID NO: 1139 - protein, 14 aa, synthetic construct
NPDPNANPNVDPNA

SEQ ID NO: 1140 - protein, 14 aa, synthetic construct
NPNVDPNANPNANP

SEQ ID NO: 1141 - protein, 14 aa, synthetic construct
NPNVDPNANPNVDP

SEQ ID NO: 1142 - protein, 14 aa, synthetic construct
NVDPNANPNANPNA

SEQ ID NO: 1143 - protein, 14 aa, synthetic construct
NVDPNANPNVDPNA

SEQ ID NO: 1144 - protein, 14 aa, synthetic construct
PADGNPDPNANPNV

SEQ ID NO: 1145 - protein, 14 aa, synthetic construct
PDPNANPNVDPNAN

SEQ ID NO: 1146 - protein, 14 aa, synthetic construct
PNANPNVDPNANPN

SEQ ID NO: 1147 - protein, 14 aa, synthetic construct
PNVDPNANPNANPN

SEQ ID NO: 1148 - protein, 14 aa, synthetic construct
PNVDPNANPNVDPN

SEQ ID NO: 1149 - protein, 14 aa, synthetic construct
VDPNANPNANPNAN

SEQ ID NO: 1150 - protein, 14 aa, synthetic construct
VDPNANPNVDPNAN

SEQ ID NO: 1151 - protein, 15 aa, synthetic construct
ADGNPDPNANPNVDP

SEQ ID NO: 1152 - protein, 15 aa, synthetic construct
ANPNVDPNANPNANP

SEQ ID NO: 1153 - protein, 15 aa, synthetic construct
ANPNVDPNANPNVDP

SEQ ID NO: 1154 - protein, 15 aa, synthetic construct
DGNPDPNANPNVDPN

SEQ ID NO: 1155 - protein, 15 aa, synthetic construct
DPNANPNANPNANPN

SEQ ID NO: 1156 - protein, 15 aa, synthetic construct
DPNANPNVDPNANPN

SEQ ID NO: 1157 - protein, 15 aa, synthetic construct
GNPDPNANPNVDPNA

SEQ ID NO: 1158 - protein, 15 aa, synthetic construct
NANPNVDPNANPNAN

SEQ ID NO: 1159 - protein, 15 aa, synthetic construct
NANPNVDPNANPNVD

SEQ ID NO: 1160 - protein, 15 aa, synthetic construct
NPDPNANPNVDPNAN

SEQ ID NO: 1161 - protein, 15 aa, synthetic construct
NPNVDPNANPNANPN

SEQ ID NO: 1162 - protein, 15 aa, synthetic construct
NPNVDPNANPNVDPN

SEQ ID NO: 1163 - protein, 15 aa, synthetic construct
NVDPNANPNANPNAN

SEQ ID NO: 1164 - protein, 15 aa, synthetic construct
NVDPNANPNVDPNAN

SEQ ID NO: 1165 - protein, 15 aa, synthetic construct
PADGNPDPNANPNVD

SEQ ID NO: 1166 - protein, 15 aa, synthetic construct
PDPNANPNVDPNANP

SEQ ID NO: 1167 - protein, 15 aa, synthetic construct
PNANPNVDPNANPNA

SEQ ID NO: 1168 - protein, 15 aa, synthetic construct
PNANPNVDPNANPNV

SEQ ID NO: 1169 - protein, 15 aa, synthetic construct
PNVDPNANPNANPNA

SEQ ID NO: 1170 - protein, 15 aa, synthetic construct
PNVDPNANPNVDPNA

SEQ ID NO: 1171 - protein, 15 aa, synthetic construct
VDPNANPNANPNANP

SEQ ID NO: 1172 - protein, 15 aa, synthetic construct
VDPNANPNVDPNANP

SEQ ID NO: 1173 - protein, 16 aa, synthetic construct
ADGNPDPNANPNVDPN

SEQ ID NO: 1174 - protein, 16 aa, synthetic construct
ANPNVDPNANPNANPN

SEQ ID NO: 1175 - protein, 16 aa, synthetic construct
ANPNVDPNANPNVDPN

SEQ ID NO: 1176 - protein, 16 aa, synthetic construct
DGNPDPNANPNVDPNA

SEQ ID NO: 1177 - protein, 16 aa, synthetic construct
DPNANPNANPNANPNA

SEQ ID NO: 1178 - protein, 16 aa, synthetic construct
DPNANPNVDPNANPNA

SEQ ID NO: 1179 - protein, 16 aa, synthetic construct
DPNANPNVDPNANPNV

SEQ ID NO: 1180 - protein, 16 aa, synthetic construct
GNPDPNANPNVDPNAN

SEQ ID NO: 1181 - protein, 16 aa, synthetic construct
NANPNVDPNANPNANP

SEQ ID NO: 1182 - protein, 16 aa, synthetic construct
NANPNVDPNANPNVDP

SEQ ID NO: 1183 - protein, 16 aa, synthetic construct
NPDPNANPNVDPNANP

SEQ ID NO: 1184 - protein, 16 aa, synthetic construct
NPNVDPNANPNANPNA

SEQ ID NO: 1185 - protein, 16 aa, synthetic construct
NPNVDPNANPNVDPNA

SEQ ID NO: 1186 - protein, 16 aa, synthetic construct
NVDPNANPNANPNANP

SEQ ID NO: 1187 - protein, 16 aa, synthetic construct
NVDPNANPNVDPNANP

SEQ ID NO: 1188 - protein, 16 aa, synthetic construct
PADGNPDPNANPNVDP

SEQ ID NO: 1189 - protein, 16 aa, synthetic construct
PDPNANPNVDPNANPN

SEQ ID NO: 1190 - protein, 16 aa, synthetic construct
PNANPNVDPNANPNAN

SEQ ID NO: 1191 - protein, 16 aa, synthetic construct
PNANPNVDPNANPNVD

SEQ ID NO: 1192 - protein, 16 aa, synthetic construct
PNVDPNANPNANPNAN

SEQ ID NO: 1193 - protein, 16 aa, synthetic construct
PNVDPNANPNVDPNAN

SEQ ID NO: 1194 - protein, 16 aa, synthetic construct
VDPNANPNANPNANPN

SEQ ID NO: 1195 - protein, 16 aa, synthetic construct
VDPNANPNVDPNANPN

SEQ ID NO: 1196 - DNA, 26 bp, synthetic construct
  source 1..26: mol_type = other DNA
tgggagattggttttgacgtttatgt

SEQ ID NO: 1197 - DNA, 30 bp, synthetic construct
  source 1..30: mol_type = other DNA
aagcattaaataaagcgaatacatccttac

SEQ ID NO: 1198 - protein, 24 aa, synthetic construct
NANPNANPNANPNANPNANPNANP

SEQ ID NO: 1199 - protein, 15 aa, synthetic construct
KQPADGNPDPNANPN

SEQ ID NO: 1200 - protein, 20 aa, synthetic construct
NVDPNANPNVDPNANPNVDP

SEQ ID NO: 1201 - protein, 15 aa, synthetic construct
KQPADJNPDPNANPN

SEQ ID NO: 1202 - protein, 4 aa, synthetic construct
  REGION >4: note = The entire sequence of amino acids 1-4 can be repeated two to five times
NVDP

SEQ ID NO: 1203 - protein, 4 aa, synthetic construct
  REGION >4: note = The entire sequence of amino acids 1-4 can be repeated twenty five or forty one times
NANP